SEQUENCE LISTING (49 sequences)

SEQ ID NO: 1 - protein, 574 aa, Artificial sequence
  source: /note=“Description of artificial sequence Synthetic polypeptide”
MELLTLKANAITTILTAVTFCFASGQNITEEFYQSTCSAVSKGYLSALRTGWYTSVITIELSNIKKNKCNGTDAKVKLIKQELDKYKNAVTELQLLMQSTQATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 2 - protein, 574 aa, Artificial sequence
  source: /note=“Description of artificial sequence Synthetic polypeptide”
MELPILKANAITTILAAVTFCFASSQNITEEFYQSTCSAVSKGYLSALRTGWYTSVITIELSNIKENKCNGTDAKVKLIKQELDKYKNAVTELQLLMQSTPAANNRARRELPRFMNYTLNNTKKTNVTLSKKRKRRFLGFLLGVGSAIASGIAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 3 - protein, 545 aa, Artificial sequence
  source: /note=“Description of artificial sequence Synthetic polypeptide”
MELLILKANAITTILTAVTFCFASGQNITEEFYQSTCSAVSKGYLSALRTGWYTSVITIELSNIKKNKCNGTDAKVKLIKQELDKYKNAVTELQLLMQSTQATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCRISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNMDYKDDDDKNSAVDMHHHHHH

SEQ ID NO: 4 - protein, 586 aa, Artificial sequence
  source: /note=“Description of artificial sequence Synthetic polypeptide”
MELLILKANAITTILTAVTFCFASGQNITEEFYQSTCSAVSKGYLSALRTGWYTSVITIELSNIKKNKCNGTDAKVKLIKQELDKYKNAVTELQLLMQSTQATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNGENLYFQGGHMKQIEDKIEEILSKIYHIENEIARIKKLIGEVDYKDDDDKGIEGRGHHHHHH

SEQ ID NO: 5 - protein, 545 aa, Artificial sequence
  source: /note=“Description of artificial sequence Synthetic polypeptide”
MELLILKANAITTILTAVTFCFASGQNITEEFYQSTCSAVSKGYLSALRTGWYTSVITIELSNIKKNKCNGTDAKVKLIKQELDKYKNAVTELQLLMQSTQATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTCCMDYKDDDDKNSAVDMHHHHHH

SEQ ID NO: 6 - DNA, 2000 bp, Artificial sequence
  source: /note=“Description of artificial sequence Synthetic polynucleotide”
ggtctatataagcagagctctctggctaactagagaacccactgcttactggcttatcgaaattaatacgactcactatagggagacccaagctggctagcgtttaaacttaagggccttcacaattgttagcgcgcatcactagttcacgatcgctaggatccattgggccctatccgcggagaatcaaaataaactctggggcaaataaccatggagctgctcatcctgaaggccaacgccatcaccaccatcctcaccgccgtgaccttctgcttcgccagcggccagaatatcaccgaggagttctaccagagcacctgcagcgccgtgagcaagggctacctgagcgccctgagaaccggctggtacaccagcgtgatcaccatcgagctgagcaacatcaagaagaacaagtgcaacggcaccgacgccaaggtgaagctcatcaagcaggagctggacaagtacaagaacgccgtgaccgagctgcagctgctcatgcagagcacccaggccaccaacaacagggccagaagggagctgccccggttcatgaactacaccctgaacaacgccaagaaaaccaacgtgaccctgagcaagaagcggaagcggagattcctgggcttcctgctgggcgtgggcagcgccatcgccagcggagtggccgtgtccaaggtgctgcacctggagggcgaggtgaacaagatcaagagcgccctgctgagcaccaacaaggccgtggtgagcctgagcaacggcgtgagcgtgctcaccagcaaggtgctggatctgaagaactacatcgacaagcagctgctgcccatcgtgaacaagcagagctgcagcatcagcaacatcgagaccgtgatcgagttccagcagaagaacaaccggctgctggagatcaccagggagttcagcgtgaacgccggcgtgaccacccccgtgagcacctacatgctcaccaacagcgagctgctgagcctcatcaacgacatgcccatcaccaacgaccagaagaagctcatgagcaacaacgtgcagatcgtgcggcagcagagctactccatcatgagcatcatcaaggaggaggtgctggcctacgtggtgcagctgcccctgtacggcgtgatcgataccccttgctggaagctgcacaccagccccctgtgcaccaccaacaccaaggagggcagcaacatctgcctcaccaggaccgatagaggctggtactgcgacaacgccggcagcgtgtcattctttccacaggccgagacctgcaaggtgcagagcaaccgggtgttctgcgacaccatgaacagcctcaccctgcccagcgaagtgaacctgtgcaacgtggacatcttcaaccccaagtacgactgcaagatcatgaccagcaagaccgacgtgagcagcagcgtgattaccagcctgggcgccatcgtgagctgctacggcaagaccaagtgcaccgccagcaacaagaaccgggggatcatcaagaccttcagcaacggctgcgactacgtgagcaacaagggcgtggataccgtgagcgtgggcaacaccctgtactacgtgaataagcaggagggcaagagcctgtacgtgaagggcgagcccatcatcaacttctacgaccccctggtgttccctagcgacgagttcgatgccagcatcagccaggtgaacgagaagatcaaccagagcctggccttcatcaggaagagcgacgagctgctgcacaatgtgaatgccggcaagagcaccaccaatatcatgatcaccacaatcatcatcgtgatcattgtgatcctgctgtccctcatcgccgtgggcctgctgctgtactgcaaggccagaagcacccctgtgaccctgtccaaggatcagctgagcggcatcaacaatatcgccttctccaactgatatagttatataaaacacaattgcatgccactcgaggcagtcgactacgagcgtttaaaccc

SEQ ID NO: 7 - DNA, 46 bp, Artificial sequence
  source: /note=“Description of artificial sequence Synthetic primer”
cttgtcatcgtcatctttataatccatattggtggtgctcttgccg

SEQ ID NO: 8 - DNA, 62 bp, Artificial sequence
  source: /note=“Description of artificial sequence Synthetic primer”
aatagcgccgtcgacatgcatcatcatcatcatcattgatatagttatataaaacacattgc

SEQ ID NO: 9 - DNA, 21 bp, Artificial sequence
  source: /note=“Description of artificial sequence Synthetic primer”
ggtgctcttgccggcattcac

SEQ ID NO: 10 - DNA, 30 bp, Artificial sequence
  source: /note=“Description of artificial sequence Synthetic primer”
tgctgcatggattataaagatgacgatgac

SEQ ID NO: 11 - DNA, 84 bp, Artificial sequence
  source: /note=“Description of artificial sequence Synthetic primer”
ccagcatcagccaggtgaacgagaagatcaaccagagcctggccttcatcaggaagagcgacgagctgctgcacaatgtgaatg

SEQ ID NO: 12 - DNA, 107 bp, Artificial sequence
  source: /note=“Description of artificial sequence Synthetic primer”
ctcaggatctcctcgatcttgtcctcgatctgcttcatgtgcccgccctgaaaatacaggttttccccattggtggtgctcttgccggcattcacattgtgcagcag

SEQ ID NO: 13 - DNA, 79 bp, Artificial sequence
  source: /note=“Description of artificial sequence Synthetic primer”
caagatcgaggagatcctgagcaagatctaccacatcgagaacgagatcgcccgcatcaagaagctgatcggcgaggtg

SEQ ID NO: 14 - DNA, 121 bp, Artificial sequence
  source: /note=“Description of artificial sequence Synthetic primer”
ctcgagtggcatgcatttggtaccgtgttttatataactatatcaatgatgatgatgatgatgcccccttccctcgatccccttgtcatcgtcatctttataatccacctcgccgatcagc

SEQ ID NO: 15 - DNA, 20 bp, Artificial sequence
  source: /note=“Description of artificial sequence Synthetic primer”
ccagcatcagccaggtgaac

SEQ ID NO: 16 - DNA, 20 bp, Artificial sequence
  source: /note=“Description of artificial sequence Synthetic primer”
ctcgagtggcatgcatttgg

SEQ ID NO: 17 - DNA, 24 bp, Artificial sequence
  source: /note=“Description of artificial sequence Synthetic primer”
aactacatcgacaagcagctgctg

SEQ ID NO: 18 - protein, 4 aa, Artificial sequence
  source: /note=“Description of artificial sequence Synthetic peptide”
RARR

SEQ ID NO: 19 - protein, 6 aa, Artificial sequence
  source: /note=“Description of artificial sequence Synthetic peptide”
RRKRKK

SEQ ID NO: 20 - protein, 10 aa, Artificial sequence
  source: /note=“Description of artificial sequence Synthetic peptide”
VLDLKNYIDK

SEQ ID NO: 21 - protein, 6 aa, Artificial sequence
  source: /note=“Description of artificial sequence Synthetic 6xHis tag”
HHHHHH

SEQ ID NO: 22 - protein, 438 aa, Human respiratory syncytial virus
FLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWFCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 23 - protein, 438 aa, Human respiratory syncytial virus
FLGFLLGVGSAIASGIAVSKVLHLEGEVNKIKNALLSTNKAVVSLSNGVSVLTSKVLDLKNYINNRLLPIVNQQSCRISNIETVIEFQQMNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRRSDELLHNVNTGKSTTNIMITTIIIVIIVVLLSLIAIGLLLYCKAKNTPVTLSKDQLSGINNIAFSK

SEQ ID NO: 24 - protein, 438 aa, Bovine respiratory syncytial virus
FLGFLLGIGSAIASGVAVSKVLHLEGEVNKIKNALLSTNKAVVSLSNGVSVLTSKVLDLKNYIDKELLPKVNNHDCKISNIATVIEFQQKNNRLLEIAREFSVNAGITTPLSTYMLTNSELLSLINDMPITNDQKKLMSSNVQIVRQQSYSIMSVVKEEVMAYVVQLPIYGVIDTPCWKLHTSPLCTTDNKEGSNICLTRTDRGWYCDNAGSVSFFPQAETCKVQSNRVFCDTMNSLTLPTDVNLCNTDIFNAKYDCKIMTSKTDISSSVITSIGAIVSCYGKTKCTASNKNRGIIKTFSNGCDYVSNRGVDTVSVGNTLYYVNKLEGKALYIKGEPIINYYDPLVFPSDEFDASIAQVNAKINQSLAFIRRSDELLHSVDVGKSTTNVVITTIIIVIVVVILMLIAVGLLFYSKTRSTPIMLGKDQLSGINNLSFSK

SEQ ID NO: 25 - protein, 437 aa, Human metapneumovirus
FVLGAIALGVATAAAVTAGVAIAKTIRLESEVTAIKNALKTTNEAVSTLGNGVRVLATAVRELKDFVSKNLTRAINKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSGKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPIKFPEDQFNVALDQVFENIENSQALVDQSNRILSSAEKGNTGFIIVIILIAVLGSSMILVSIFIIIKKTKKPTGAPPELSGVTNNGFIPHS

SEQ ID NO: 26 - protein, 443 aa, Human parainfluenza virus 1
FFGAVIGTIALGVATAAQITAGIALAEAREARKDIALIKDSIVKTHNSVELIQRGIGEQIIALKTLQDFVNDEIRPAIGELRCETTALKLGIKLTQHYSELATAFSSNLGTIGEKSLTLQALSSLYSANITEILSTTKKDKSDIYDIIYTEQVKGTVIDVDLEKYMVTLLVKIPILSEIPGVLIYRASSISYNIEGEEWHVAIPNYIINKASSLGGADVTNCIESKLAYICPRDPTQLIPDNQQKCILGDVSKCPVTKVINNLVPKFAFINGGVVANCIASTCTCGTNRIPVNQDRSRGVTFLTYTNCGLIGINGIELYANKRGRDTTWGNQIIKVGPAVSIRPVDISLNLASATNFLEESKTELMKARAIISAVGGWHNTESTQIIMIIIVCILIIIICGILYYLYRVRRLLVMINSTHNSPVNAYTLESRMRNPYMGNNSN

SEQ ID NO: 27 - protein, 430 aa, Human parainfluenza virus 3
FFGGVIGTIALGVATSAQITAAVALVEAKQARSDIEKLKEAIRDTNKAVQSVQSSIGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIHNREWYIPLPSHIMTKGAFLGGADVKECIEAFSSYICPSDPGFVLNHEMESCLSGNISQCPRTTITSDIVPRYAFVNGGVVANCITTTCTCNGIGNRINQPPNQGVKIITHKECSTIGINGMLFNTNKEGTLAFYTPDDITLNNSVALDPIDISIELNKAKSDLEESKEWIRKSNQKLDSIGNWHQSSTTIIIILMMIIILFIINITIITIAIKYYRIQKRNQMDQNDKPYVLTNK

SEQ ID NO: 28 - protein, 437 aa, Nipah virus
LAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTYSRLEDRRVRPTSSGDLYYIGT

SEQ ID NO: 29 - protein, 438 aa, Measles virus
FAGVVLAGAALGVATAAQITAGIALHQSMLNSQAIDNLRASLETTNQAIEAIRQAGQEMILAVQGVQDYINNELIPSMNQLSCDLIGQKLGLKLLRYYTEILSLFGPSLRDPISAEISIQALSYALGGDINKVLEKLGYSGGDLLGILESRGIKARITHVDTESYFIVLSIAYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPISLERLDVGTNLGNAIAKLEDAKELLESSDQILRSMKGLSSTSIVYILIAVCLGGLIGIPALICCCRGRCNKKGEQVGMSRPGLKPDLTGTSKSYVRSL

SEQ ID NO: 30 - protein, 437 aa, Newcastle disease virus
LIGAIIGGVALGVATAAQITAASALIQANQNAANILRLKESIAATNEAVHEVTNGLSQLAVAVGKMQQFVNDQFNKTAQELDCIKITQQVGVELNLYLTELTTVFGPQITSPALTQLTIQALYNLAGGNMDYLLTKLGVGNNQLSSLISSGLITGNPILYDSQTQLLGIQVTLPSVGNLNNMRATYLETLSVSTTKGFASALVPKVVTQVGSVIEELDTSYCIETDLDLYCTRIVTFPMSPGIYSCLSGNTSACMYSKTEGALTTPYMTLKGSVIANCKMTTCRCADPPGIISQNYGEAVSLIDRQSCNILSLDGITLRLSGEFDATYQKNISIQDSQVIVTGNLDISTELGNVNNSISNALDKLEESNSKLDKVNVKLTSTSALITYIVLTVISLVCGILSLVLACYLMYKQKAQQKTLLWLGNNTLDQMRATTKM

SEQ ID NO: 31 - protein, 440 aa, Human parainfluenza virus 4
FFGAVIGTIALGVATAAQVTAAIGLAKAQENAKLILTLKKAATETNEAVRDLANSNKIVVKMISAIQNQINTIIQPAIDQINCQIKDLQVANILNLYLTEITTVFHNQLTNPALESISIQALKSLLGPTLPEVLSKLDLNNISAASVMASGLIKGQIIAVDIPTMTLVLMVQIPSISPLRQAKIIDLTSITIHTNSQEVQAVVPARFLEIGSEILGFDGSVCQITKDTIFCPYNDAYVLPIQQKRCLQGQTRDCVFTPVAGTFPRRFLTTYGTIVANCRDLVCSCLRPPQIIYQPDENPVTIIDKDLCTTLTLDSITIEIQKSINSTFRREVVLESTQVRSLTPLDLSTDLNQYNQLLKSAEDHIQRSTDYLNSINPSIVNNNAIIILIILCILLILTVTICIIWLKYLTKEVKNVARNQRLNRDADLFYKIPSQIPVPR

SEQ ID NO: 32 - protein, 436 aa, Mumps virus
FAGIAIGIAALGVATAAQVTAAVSLVQAQTNARAIAAMKNSIQATNRAVFEVKEGTQRLAIAVQAIQDHINTIMNTQLNNMSCQILDNQLATSLGLYLTELTTVFQPQLINPALSPISIQALRSLLGSMTPAVVQATLSTSISAAEILSAGLMEGQIVSVLLDEMQMIVKINIPTIVTQSNALVIDFYSISSFINNQESIIQLPDRILEIGNEQWSYPAKNCKLTRHHIFCQYNEAERLSLESKLCLAGNISACVFSPIAGSYMRRFVALDGTIVANCRSLTCLCKSPSYPIYQPDHHAVTTIDLTACQTLSLDGLDFSIVSLSNITYAENLTISLSQTINTQPIDISTELSKVNASLQNAVKYIKESNHQLQSVNVNSKIGAIIVAALVLSILSIIISLLFCCWAYVATKEIRRINFKTNHINTISSSVDDLIRY

SEQ ID NO: 33 - protein, 445 aa, Human parainfluenza virus 2
FAGVVVGLAALGVATAAQITAAVAIVKANANAAAINNLASSIQSTNKAVSDVIDASRTIATAVQAIQDRINGAIVNGITSASCRAHDALIGSILNLYLTELTTIFHNQITNPALTPLSIQALRILLGSTLPIVIESKLNTNFNTAELLSSGLLTGQIISISPMYMQMLIQINVPTFIMQPGAKVIDLIAISANHKLQEVVVQVPNRILEYANELQNYPANDCVVTPNSVFCRYNEGSPIPESQYQCLRGNLNSCTFTPIIGNFLKRFAFANGVLYANCKSLLCRCADPPHVVSQDDTQGISIIDIKRCSEMMLDTFSFRITSTFNATYVTDFSMINANIVHLSPLDLSNQINSINKSLKSAEDWIADSNFFANQARTAKTLYSLSAIALILSVITLVVVGLLIAYIIKLVSQIHQFRSLAATTMFHRENPAFFSKNNHGNIYGIS

SEQ ID NO: 34 - protein, 427 aa, Simian virus 5
FAGVVIGLAALGVATAAQVTAAVALVKANENAAAILNLKNAIQKTNAAVADVVQATQSLGTAVQAVQDHINSVVSPAITAANCKAQDAIIGSILNLYLTELTTIFHNQITNPALSPITIQALRILLGSTLPTVVEKSFNTQISAAELLSSGLLTGQIVGLDLTYMQMVIKIELPTLTVQPATQIIDLATISAFINNQEVMAQLPTRVMVTGSLIQAYPASQCTITPNTVYCRYNDAQVLSDDTMACLQGNLTRCTFSPVVGSFLTRFVLFDGIVYANCRSMLCKCMQPAAVILQPSSSPVTVIDMYKCVSLQLDNLRFTITQLANVTYNSTIKLESSQILSIDPLDISQNLAAVNKSLSDALQHLAQSDTYLSAITSATTTSVLSIIAICLGSLGLILIILLSVVVWKLLTIVVANRNRMENFVYHK

SEQ ID NO: 35 - protein, 4 aa, Artificial sequence
  source: /note=“Description of artificial sequence Synthetic peptide”
YDSL

SEQ ID NO: 36 - protein, 557 aa, Artificial sequence
  source: /note=“Description of artificial sequence Synthetic polypeptide”
MELLILKANAITTILTAVTFCFASGQNITEEFYQSTCSAVSKGYLSALRTGWYTSVITIELSNIKKNKCNGTDAKVKLIKQELDKYKNAVTELQLLMQSTQATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELIIIVIIVILLSLIAVGLLLYCKARSTPVTLSKDQLSGINNIAFSN

SEQ ID NO: 37 - DNA, 7257 bp, Artificial sequence
  source: /note=“Description of artificial sequence Synthetic construct”
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagggccttcacaattgttagcgcgcatcactagttcacgatcgctaggatccattgggccctatccgcggagaatcaaaataaactctggggcaaataaccatggagctgctcatcctgaaggccaacgccatcaccaccatcctcaccgccgtgaccttctgcttcgccagcggccagaatatcaccgaggagttctaccagagcacctgcagcgccgtgagcaagggctacctgagcgccctgagaaccggctggtacaccagcgtgatcaccatcgagctgagcaacatcaagaagaacaagtgcaacggcaccgacgccaaggtgaagctcatcaagcaggagctggacaagtacaagaacgccgtgaccgagctgcagctgctcatgcagagcacccaggccaccaacaacagggccagaagggagctgccccggttcatgaactacaccctgaacaacgccaagaaaaccaacgtgaccctgagcaagaagcggaagcggagattcctgggcttcctgctgggcgtgggcagcgccatcgccagcggagtggccgtgtccaaggtgctgcacctggagggcgaggtgaacaagatcaagagcgccctgctgagcaccaacaaggccgtggtgagcctgagcaacggcgtgagcgtgctcaccagcaaggtgctggatctgaagaactacatcgacaagcagctgctgcccatcgtgaacaagcagagctgcagcatcagcaacatcgagaccgtgatcgagttccagcagaagaacaaccggctgctggagatcaccagggagttcagcgtgaacgccggcgtgaccacccccgtgagcacctacatgctcaccaacagcgagctgctgagcctcatcaacgacatgcccatcaccaacgaccagaagaagctcatgagcaacaacgtgcagatcgtgcggcagcagagctactccatcatgagcatcatcaaggaggaggtgctggcctacgtggtgcagctgcccctgtacggcgtgatcgataccccttgctggaagctgcacaccagccccctgtgcaccaccaacaccaaggagggcagcaacatctgcctcaccaggaccgatagaggctggtactgcgacaacgccggcagcgtgtcattctttccacaggccgagacctgcaaggtgcagagcaaccgggtgttctgcgacaccatgaacagcctcaccctgcccagcgaagtgaacctgtgcaacgtggacatcttcaaccccaagtacgactgcaagatcatgaccagcaagaccgacgtgagcagcagcgtgattaccagcctgggcgccatcgtgagctgctacggcaagaccaagtgcaccgccagcaacaagaaccgggggatcatcaagaccttcagcaacggctgcgactacgtgagcaacaagggcgtggataccgtgagcgtgggcaacaccctgtactacgtgaataagcaggagggcaagagcctgtacgtgaagggcgagcccatcatcaacttctacgaccccctggtgttccctagcgacgagttcgatgccagcatcagccaggtgaacgagaagatcaaccagagcctggccttcatcaggaagagcgacgagctgctgcacaatgtgaatgccggcaagagcaccaccaatggggaaaacctgtattttcagggcgggcacatgaagcagatcgaggacaagatcgaggagatcctgagcaagatctaccacatcgagaacgagatcgcccgcatcaagaagctgatcggcgaggtggattataaagatgacgatgacaaggggatcgagggaagggggcatcatcatcatcatcattgatatagttatataaaacacggtaccaaatgcatgccactcgaggcagtcgactacgagcgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaagacgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatggttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 38 - DNA, 7128 bp, Artificial sequence
  source: /note=“Description of artificial sequence Synthetic construct”
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagggccttcacaattgttagcgcgcatcactagttcacgatcgctaggatccattgggccctatccgcggagaatcaaaataaactctggggcaaataaccatggagctgctcatcctgaaggccaacgccatcaccaccatcctcaccgccgtgaccttctgcttcgccagcggccagaatatcaccgaggagttctaccagagcacctgcagcgccgtgagcaagggctacctgagcgccctgagaaccggctggtacaccagcgtgatcaccatcgagctgagcaacatcaagaagaacaagtgcaacggcaccgacgccaaggtgaagctcatcaagcaggagctggacaagtacaagaacgccgtgaccgagctgcagctgctcatgcagagcactcaggccaccaacaacagggccagaagggagctgccccggttcatgaactacaccctgaacaacgccaagaaaaccaacgtgaccctgagcaagaagcggaagcggagattcctgggcttcctgctgggcgtgggcagcgccatcgccagcggagtggccgtgtccaaggtgctgcacctggagggcgaggtgaacaagatcaagagcgccctgctgagcaccaacaaggccgtggtgagcctgagcaacggcgtgagcgtgctcaccagcaaggtgctggatctgaagaactacatcgacaagcagctgctgcccatcgtgaacaagcagagctgcagcatcagcaacatcgagaccgtgatcgagttccagcagaagaacaaccggctgctggagatcaccagggagttcagcgtgaacgccggcgtgaccacccccgtgagcacctacatgctcaccaacagcgagctgctgagcctcatcaacgacatgcccatcaccaacgaccagaagaagctcatgagcaacaacgtgcagatcgtgcggcagcagagctactccatcatgagcatcatcaaggaggaggtgctggcctacgtggtgcagctgcccctgtacggcgtgatcgataccccttgctggaagctgcacaccagccccctgtgcaccaccaacaccaaggagggcagcaacatctgcctcaccaggaccgatagaggctggtactgcgacaacgccggcagcgtgtcattctttccacaggccgagacctgcaaggtgcagagcaaccgggtgttctgcgacaccatgaacagcctcaccctgcccagcgaagtgaacctgtgcaacgtggacatcttcaaccccaagtacgactgcaagatcatgaccagcaagaccgacgtgagcagcagcgtgattaccagcctgggcgccatcgtgagctgctacggcaagaccaagtgcaccgccagcaacaagaaccgggggatcatcaagaccttcagcaacggctgcgactacgtgagcaacaagggcgtggataccgtgagcgtgggcaacaccctgtactacgtgaataagcaggagggcaagagcctgtacgtgaagggcgagcccatcatcaacttctacgaccccctggtgttccctagcgacgagttcgatgccagcatcagccaggtgaacgagaagatcaaccagagcctggccttcatcaggaagagcgacgagctgctgcacaatgtgaatgccggcaagagcacctgctgcatggattataaagatgacgatgacaagaatagcgccgtcgacatgcatcatcatcatcatcattgatatagttatataaaacacaattgcatgccactcgaggcagtcgactacgagcgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaagacgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatggttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 39 - DNA, 7128 bp, Artificial sequence
  source: /note=“Description of artificial sequence Synthetic construct”
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagggccttcacaattgttagcgcgcatcactagttcacgatcgctaggatccattgggccctatccgcggagaatcaaaataaactctggggcaaataaccatggagctgctcatcctgaaggccaacgccatcaccaccatcctcaccgccgtgaccttctgcttcgccagcggccagaatatcaccgaggagttctaccagagcacctgcagcgccgtgagcaagggctacctgagcgccctgagaaccggctggtacaccagcgtgatcaccatcgagctgagcaacatcaagaagaacaagtgcaacggcaccgacgccaaggtgaagctcatcaagcaggagctggacaagtacaagaacgccgtgaccgagctgcagctgctcatgcagagcacccaggccaccaacaacagggccagaagggagctgccccggttcatgaactacaccctgaacaacgccaagaaaaccaacgtgaccctgagcaagaagcggaagcggagattcctgggcttcctgctgggcgtgggcagcgccatcgccagcggagtggccgtgtccaaggtgctgcacctggagggcgaggtgaacaagatcaagagcgccctgctgagcaccaacaaggccgtggtgagcctgagcaacggcgtgagcgtgctcaccagcaaggtgctggatctgaagaactacatcgacaagcagctgctgcccatcgtgaacaagcagagctgcagcatcagcaacatcgagaccgtgatcgagttccagcagaagaacaaccggctgctggagatcaccagggagttcagcgtgaacgccggcgtgaccacccccgtgagcacctacatgctcaccaacagcgagctgctgagcctcatcaacgacatgcccatcaccaacgaccagaagaagctcatgagcaacaacgtgcagatcgtgcggcagcagagctactccatcatgagcatcatcaaggaggaggtgctggcctacgtggtgcagctgcccctgtacggcgtgatcgataccccttgctggaagctgcacaccagccccctgtgcaccaccaacaccaaggagggcagcaacatctgcctcaccaggaccgatagaggctggtactgcgacaacgccggcagcgtgtcattctttccacaggccgagacctgcaaggtgcagagcaaccgggtgttctgcgacaccatgaacagcctcaccctgcccagcgaagtgaacctgtgcaacgtggacatcttcaaccccaagtacgactgcaagatcatgaccagcaagaccgacgtgagcagcagcgtgattaccagcctgggcgccatcgtgagctgctacggcaagaccaagtgcaccgccagcaacaagaaccgggggatcatcaagaccttcagcaacggctgcgactacgtgagcaacaagggcgtggataccgtgagcgtgggcaacaccctgtactacgtgaataagcaggagggcaagagcctgtacgtgaagggcgagcccatcatcaacttctacgaccccctggtgttccctagcgacgagttcgatgccagcatcagccaggtgaacgagaagatcaaccagagcctggccttcatcaggaagagcgacgagctgctgcacaatgtgaatgccggcaagagcaccaccaatatggattataaagatgacgatgacaagaatagcgccgtcgacatgcatcatcatcatcatcattgatatagttatataaaacacaattgcatgccactcgaggcagtcgactacgagcgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaagacgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatggttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 40 - protein, 13 aa, Artificial sequence
  source: /note=“Description of artificial sequence Synthetic peptide”
VXXXXXYXXXXXR

SEQ ID NO: 41 - protein, 13 aa, Artificial sequence
  source: /note=“Description of artificial sequence Synthetic peptide”
VXXXXXYXXXXXD

SEQ ID NO: 42 - protein, 10 aa, Artificial sequence
  source: /note=“Description of artificial sequence Synthetic peptide”
VLDLKNYIDR

SEQ ID NO: 43 - protein, 10 aa, Artificial sequence
  source: /note=“Description of artificial sequence Synthetic peptide”
VLDIKNYIDK

SEQ ID NO: 44 - protein, 10 aa, Artificial sequence
  source: /note=“Description of artificial sequence Synthetic peptide”
ILDLKNYIDK

SEQ ID NO: 45 - protein, 11 aa, Artificial sequence
  source: /note=“Description of artificial sequence Synthetic peptide”
VLDLKNYINNR

SEQ ID NO: 46 - protein, 10 aa, Artificial sequence
  source: /note=“Description of artificial sequence Synthetic peptide”
VRELKDFVSK

SEQ ID NO: 47 - protein, 13 aa, Artificial sequence
  source: /note=“Description of artificial sequence Synthetic peptide”
LKTLQDFVNDEIR

SEQ ID NO: 48 - protein, 7 aa, Artificial sequence
  source: /note=“Description of artificial sequence Synthetic peptide”
VQDYVNK

SEQ ID NO: 49 - protein, 7 aa, Artificial sequence
  source: /note=“Description of artificial sequence Synthetic peptide”
VNDQFNK

(19) United States
(12) Patent Application Publication
(10) Pub. No.: US 2010/0261155 A1
(43) Pub. Date: Oct. 14, 2010

(54) METHODS AND COMPOSITIONS RELATING TO VIRAL FUSION PROTEINS

(75) Inventors: Mark E. Peeples, Bexley, OH (US); Matthew Kennedy, Groveport, OH (US); William Ray, Columbus, OH (US)

Correspondence Address: CALFEE HALTER & GRISWOLD, LLP, 800 SUPERIOR AVENUE, SUITE 1400, CLEVELAND, OH, 44114, US

(73) Assignee: NATIONWIDE CHILDREN'S HOSPITAL, INC., Columbus, OH (US)

(21) Appl. No.: 12/663,418

(22) PCT Filed: Jun. 6, 2008
(86) PCT No.: PCT/US08/66223

§ 371 Date: Jun. 4, 2010

Related U.S. Application Data
(60) Provisional application No. 60/942,456, filed on Jun. 6, 2007.

(51) Int. Cl. C12Q 1/70; C07K 14/005

(52) U.S. Cl. 435/5; 530/350

(57) ABSTRACT

Provided herein are isolated paramyxovirus pre-triggered fusion proteins, or functional fragments thereof, which contain one or more CRAC domains in a location that is away from the transmembrane domain. Also provided herein is a computer model of the structure of the pre-triggered F protein. Compositions that directly or indirectly bind and interfere with the normal activity or binding of the pre-triggered F proteins, or the CRAC domains, are useful as antiviral agents in the treatment of paramyxovirus infections. Thus, disclosed herein are methods of screening for antiviral agents, using the isolated pre-triggered F protein, or functional fragments thereof.

### CROSS-REFERENCE TO RELATED APPLICATIONS

This application is the national stage of International Application No. PCT/US 08/66223, filed Jun. 6, 2008, which claims the benefit of U.S. Provisional Application No. 60/942,456, filed Jun. 6, 2007, the entire contents of which are incorporated herein by reference.

### STATEMENT REGARDING FEDERALLY SPONSORED RESEARCH

This invention was made, at least in part, with government support under National Institutes of Health Grant No: AI047213. The U.S. government may have certain rights in the invention.

### BACKGROUND

To initiate infection, viruses bind to one or more receptors on a target cell. The second step is entry. Many viruses are enveloped with a lipid membrane derived from the cell in which they were produced. Following attachment, these enveloped viruses fuse their membrane with a target cell membrane to allow the contents of the virion, including the viral genome, to enter the cell.

Paramyxoviruses are viruses of the Paramyxoviridae family of the Mononegavirales order. They are negative-sense single-stranded RNA viruses responsible for a number of human and animal diseases.

The Paramyxovirus family includes 2 subfamilies: (i) Paramyxovirus: including parainfluenza virus (PIV) 1-4, Newcastle disease virus (NDV), Nipah virus, measles virus and mumps virus; (ii) Pneumovirus: including human respiratory syncytial virus (RSV), bovine RSV and human metapneumovirus (hMPV). Parainfluenza viruses and RSV produce acute respiratory diseases of the upper and lower respiratory tracts, whereas measles and mumps viruses cause systemic disease.

RSV causes respiratory tract infections in patients of all ages. It is the major cause of lower respiratory tract infection during infancy and childhood. In temperate climates there is an annual epidemic during the winter months. In tropical climates, infection is most common during the rainy season. In the United States, 60% of infants are infected during their first RSV season, and nearly all children will have been infected with the virus by 2-3 years of age. Natural infection with RSV does not induce protective immunity, and thus people can be infected multiple times. Sometimes an infant can become symptomatically infected more than once even within a single RSV season. More recently, RSV infections have been found to be frequent among elderly patients as well. As the virus is ubiquitous, avoidance of infection is not possible. There is currently no vaccine or specific treatments against RSV. The failure in developing a vaccine has led to renewed interest in the pathogenesis of the disease.

There is a need, generally, for methods to identify antiviral agents that inhibit the activity of fusion proteins, or reduce the infectivity of paramyxoviruses, such as RSV (human and bovine), hMPV, PIV1 and 3 and NDV.

### BRIEF SUMMARY

Provided herein is a pre-triggered soluble fusion (F) protein of a virus in the paramyxovirus family, wherein the soluble fusion protein lacks a transmembrane domain and a cytoplasmic tail domain and includes a CRAC1 domain. The soluble fusion protein is in a pre-triggered conformation and can be triggered when exposed to a triggering event.

Also provided is a functional fragment of an RSV soluble fusion protein, comprising a first and a second peptide linked to form a dimer peptide. The first and second peptides include, respectively, a sequence that is at least 90% identical to amino acids 37-69 and 156-440 of SEQ ID NO: 1, and the second peptide includes a CRAC1 domain.

Also contemplated are methods of screening for a candidate paramyxovirus antiviral agent, including the steps of: (i) contacting a test agent with a soluble F protein of a paramyxovirus described above and (ii) detecting a structural indicator of the soluble pre-triggered F protein. A change in the structural indicator of the soluble pre-triggered F protein in the presence of the test agent as compared to the absence of the test agent indicates that the agent is a candidate antiviral agent against the paramyxovirus.

Another method contemplated herein is a method of screening for a candidate paramyxovirus antiviral agent that includes the steps of: (i) contacting a test agent with a soluble F protein of the paramyxovirus, described above, to form a test sF protein; (ii) exposing the test sF protein to a triggering event; and (iii) assessing a structural indicator of the test sF protein before and after exposure to the triggering event. In this method, an absence of a change in the structural indicator of the test sF protein after exposure to the triggering event indicates that the agent is a candidate antiviral agent against the paramyxovirus.

Also provided is a method of screening for a candidate antiviral agent against RSV, including the steps of: (i) contacting a test agent with a functional fragment of a soluble pre-triggered F protein of RSV, described above; and (ii) detecting a structural indicator of the functional fragment. A change in the structural indicator of the functional fragment in the presence of the test agent as compared to the absence of the test agent indicates that the agent is a candidate antiviral agent against RSV.

Also included is a method of screening for a candidate antiviral agent against RSV, comprising the steps of: (i) contacting a test agent with a functional fragment of a soluble pre-triggered F protein of RSV, as described above, to form a test sF protein; (ii) exposing the test sF protein to a triggering event; and (iii) assessing a structural indicator of the test sF protein before and after exposure to the triggering event. In this method, the absence of a change in the structural indicator of the test sF protein after exposure to the triggering event indicates that the agent is a candidate antiviral agent against RSV.

### BRIEF DESCRIPTION OF THE DRAWINGS

FIG. 1 is a cartoon depiction of the RSV F protein processing in a cell. F0 is the precursor protein that is cleaved at two furin cleavage sites (fcs) to yield the fully functional F1+F2 disulfide-linked dimer. Heptad repeats HR1 and HR2 form α-helical structures critical for completing membrane fusion [RARR (SEQ ID NO: 18); RRKRKK (SEQ ID NO: 19)].

FIG. 2 shows a model of F protein refolding to initiate fusion. The N terminal heptad repeat (HR1) is actually comprised of 3 short α-helices connected by non-helical peptides, initially, that re-fold into a long helix upon triggering.

FIG. 3 shows models of the pre-triggered and post-triggered RSV F protein monomer. (A) The pre-triggered F protein N-terminus is the fusion peptide (gray: middle, left). The segment that will become heptad repeat 1 (HR1) follows the fusion peptide and is composed of three helices with connecting peptides (1, 2 and 3). The central helix contains the CRAC1 domain (2). HR2 is another helix (4) (bottom), terminating in the transmembrane domain (gray) (5) that anchors the F protein in the virion membrane. (B) In the post-triggered RSV F protein monomer, HR1 is the long helix (6) on the right side of the molecule. HR2 is the helix (4) appears to cross HR1 from left to right. The fusion peptide would be connected to the HR1 helix, as indicated, and extend downward. The transmembrane domain would be connected to the HR2 helix and extend downward, through the virion membrane. Disulfide bonds are indicated (light gray balls) and the 2 N-linked glycosylation sites are indicated (N-link 2 and N-link 3, dark gray balls). The third N-linked site would be at the N-terminus of F2 if the previous amino acid, asparagine, had been included in the structure.

FIG. 4 shows models of the pre-triggered (A) and post-triggered (B) RSV F protein. These models are the same as those presented in FIG. 3, except that the CRAC1 domain is highlighted in ball-and-stick form. The dark balls (11 and 12) denote the defining amino acids of the CRAC1 domain and the dark balls on the other side of the CRAC helix denote the amino acids on the back side of the CRAC1 domain. The amino acids of the CRAC3 domain are denoted with light gray balls (10) in the middle right of the pre-triggered model and the upper left of the post-triggered model. This region cradles the fusion peptide from the neighboring monomer in the F protein trimer, before the fusion peptide is released by cleavage at fcs1.

FIG. 5 shows a model of the pre-triggered (A) and post-triggered (B) RSV F protein trimer. Two of the monomers are presented in the space-filling mode (one is light gray, the other is dark gray). The third monomer is presented in cartoon form. The pre-triggered molecule (A) is oriented such that the hole in the side of the F protein trimer head into which the fusion peptide slips after cleavage is visible. The fusion peptide from the cartoon monomer (white helix) is partially visible to the left of the hole. This is the position of the fusion peptide before cleavage. Note that the stalk (7) of the pre-triggered form (A) is composed of the three HR2 domains only, while the stalk of the post-triggered form (B) is a 6-helix bundle, with the HR1 trimer inside and the HR2 helices on the outside. Also note that the HR1 and HR2 from the same monomer do not interact in the 6-helix bundle.

FIG. 6 shows a view of the top of the RSV F protein trimer model. (A) Through the central pocket in the crown of the trimer a darker area is visible, representing the bottom of the pocket. The cholesterol-binding amino acids (8) of the CRAC domain are in (B) medium gray and in (C) a highlighted medium gray surface net. The other 2 CRAC1 domains from the other 2 monomers are also netted and together these three CRAC1 domains line the pocket.

FIG. 7 shows a close-up view of the CRAC1 domain and the amino acids that interact with the back side of the CRAC1 α-helix. The cholesterol-binding amino acids (K201, Y198, L195) are dark gray spheres. Below them are the spheres representing the amino acids on the back side of the CRAC1 helix (I199, D200, K196, N197) (medium gray) and below them are spheres representing the amino acids (white) that interact with these back-side amino acids. These interacting amino acids are on the neighboring loop (N175, K176, A177) and on F2 (N63). The neighboring monomer is in cartoon is covered by a net representing its surface, and the third monomer is in a space-filling model.

FIG. 8 is a cartoon depicting types of protein-protein interactions between the back of the CRAC1 helix (SEQ ID NO: 20) and the adjacent peptide. Another interaction between the back of the CRAC1 helix and the adjacent peptide and an amino acid in the F2 protein.

FIG. 9 is a sequence comparison of the F protein from RSV strains A2 (SEQ ID No: 1) and Long (SEQ ID No: 2). Both sequences were determined in our laboratory from virus provided by the American Type Culture Collection (ATCC). Amino acids of Long strain that are identical to A2 strain are indicated by dots, and differences are indicated with a letter representing the amino acid at that position. The F protein is cleaved at two sites fcs1 and fcs2, releasing three peptide products: F2 (double overlined, equal thickness), pep27 (single overlined) and F1 (double overlined, unequal thickness). Two disulfide bonds link F1 and F2 after the cleavage of this protein. The F protein is a trimer. During membrane fusion process, the two heptad repeats, HR1 and HR2 form an anti-parallel 6-helix bundle.

FIG. 10 depicts cartoons of the mature RSV F protein and the three RSV soluble fusion (sF) protein constructs, SC-2, HC-1 and sMP340-A, used in our studies. 6HIS (SEQ ID NO: 21) and FLAG are tags, Factor Xa and TEV are specific protease sites, and GCNt is a self-trimerizing clamp.

FIG. 11 is a sequence comparison of the RSV sF proteins used in our studies. The RSV D46 F protein sequence was used to generate the sF proteins. For SC-2 (SEQ ID No. 3) and sMP340-A (SEQ ID No. 4), the F protein sequence was truncated after amino acid 524. HC-1 (SEQ ID No. 5) was truncated after amino acid 522, followed by the addition of two cysteine residues. The following sequences were appended to the C terminus of the sF proteins in the positions shown in the figure: TEV (tobacco etch virus protease site); GCNt (a trimeric coiled-coil domain); the FLAG epitope tag; FXa (Factor Xa protease site); and/or the 6HIS epitope tag (SEQ ID NO: 21).

FIG. 12 shows a western blot analysis of sF protein produced in and secreted from transfected human embryonic kidney 293T cells. A 12 ul aliquot of media from SC-2 and sMP340-A transfected cells were reduced and separated by sodium dodecyl sulfate-polyacrylamide gel electrophoresis, transferred to a nylon membrane and stained with the FLAG M2 MAb followed by anti-mouse-HRP and detection by chemiluminescence. The initial protein produced from these genes is the uncleaved precursor sF0 protein. As sF0 traverses the Golgi, it is cleaved in two places by furin to yield the mature sF1+F2 protein. When the sF1+F2 protein is reduced before electrophoresis, by treatment with 2-mercaptoethanol, the sF1 and F2 proteins are separated. Both the sF0 and sF1 proteins are detected in the cell lysates (C) because the FLAG tag is located at the C terminus of the sF0 protein, which is also the C terminus of the F1 protein. Only the sF1 protein was found in the supernatant media (S), indicating that only the fully cleaved form of the sF protein was secreted. The C lanes were loaded with 10× more cell equivalents than the S lanes. Since the amount of sF0 and sF1 protein in the cell lysates appears to be approximately equal to the amount of sF1 in the supernatant and since 10× more cell equivalents were loaded in the C lanes, 80% to 90% of the sF protein produced in the cell was secreted. The minor species smaller than sF1 were not identified but probably represent minor breakdown products of the sF proteins.

FIG. 13 shows Nickel column purified RSV sF protein (SC-2) analyzed by SDS-PAGE in the presence and absence of 2-mercaptoethanol and stained with Coomassie Blue. Serial 2-fold dilutions of sF protein were loaded.

FIG. 14 shows a sucrose gradient analysis of sMP340-A and SC-2 sF proteins that had been stored at −20° C. before analysis. Both proteins were thawed at room temperature and incubated at 4° C. or 50° C. for 1 hour before loading on the top of a 15% to 55% linear sucrose gradient. The gradients were ultracentrifuged in an SW41 rotor at 41,000 rpm for 20 hours and fractionated into 1 ml fractions. The protein in each fraction was TCA precipitated, separated by SDS-PAGE and detected by western blot with FLAG M2 MAb, anti-mouse-HRP, and chemiluminescence.

FIG. 15 shows analysis of RSV sF protein aggregation state by velocity sedimentation on a sucrose gradient. Freshly prepared and purified SC-2, HC-1 and sMP340-A sF proteins were incubated at 4° C. or 50° C. for 1 hour before loading on a 15% to 55% linear sucrose gradient for analysis as described in FIG. 14.

FIG. 16 shows the reactivity of 11 neutralizing MAbs with RSV sF proteins before and after mild heat treatment. SC-2 and sMP340-A sF proteins were metabolically labeled with ³⁵S-Met/Cys and incubated for one hr at 4° C. or 50° C. followed by immunoprecipitation and autoradiography.

FIG. 17 shows association of an RSV sF protein with POPC:POPE:cholesterol (8:2:5) large uni-lamellar liposomes. After incubating the SC-2 sF protein at 4° C. (A) or 37° C. (B) for 1 hour in the presence of liposomes, sucrose was added to a final density of 50% in 1 ml, overlayed with 1 ml each of 40%, 30% and 20% sucrose and buffer, incubated at 20° C. for 1 hour to allow the gradient to form by diffusion, and centrifuged in an SW55 Ti rotor at 55,000 rpm for 2.5 hr. Each fraction was treated with Triton-X100 and the protein was concentrated and analyzed as described in the legend to FIG. 14. T indicates top and B indicates bottom of the gradient. A third reaction (C) was treated at pH 11 for 1 hour at 37° C. following the initial 1 hour incubation at 37° C. to determine if the sF protein association with the liposomes was stable. The pH 11 treatment removes proteins that peripherally associated with liposomes.

FIG. 18 shows fusion of liposomes with virions from recombinant green fluorescent protein expressing RSV containing the F glycoprotein as the only viral glycoprotein (rgRSV-F). Sucrose gradient-purified rgRSV-F was labeled with R18 lipid dye at self-quenching concentrations, separated from the free dye, and mixed with POPC (1-Palmitoyl-2-Oleoyl-sn-Glycero-3-Phosphocholine) liposomes (black squares, lower cluster), or with POPC liposomes with 30% cholesterol (gray tringles, upper cluster). Incubation at 37° C. resulted in fluorescence due to fusion of the virion membrane with the liposome membrane and subsequent dilution of the R18 dye.

FIG. 19a shows effects of single amino acid changes within the RSV F protein CRAC1 domain on cell-cell fusion. Human embryonic kidney 293T cells were co-transfected with pcDNA3.1 plasmids (Invitrogen) expressing the RSV F protein and the green fluorescent protein (A) Optimized wild-type strain A, D46 RSV F protein was express from plasmid MP340. (B-L) Single point mutations in MP340 that changed the individual amino acids as indicated were also expressed. Cells were photographed at 48 hours post-transfection.

FIG. 19b shows the effects of both central tryosines were changed of the CRAC3 domain to alanine Cells infected with wild-type D46 F protein (A) was compared to a CRAC3 mutant (B). In this experiment, pictures were taken 23 hours after transfection.

FIG. 20 is an alignment of certain paramyxovirus F1 protein sequences (SEQ ID NOS 22-34, respectively, in order of appearance). The amino acid sequences presented in this figure are a portion of the full length F protein starting immediately after the fusion peptide sequence, i.e., in RSV, amino acid 1 in FIG. 20 corresponds to amino acid 137 in SEQ ID NO: 1. Traditional CRAC motifs that end with a basic amino acid (LN-X₁₋₅-Y/F/W-X₁₋₅-R/K) are highlighted with dark grey. Proposed CRAC domains that end with an acidic amino acid (D/E) are highlighted in medium grey. CRAC domains with phenylalanine (F) or tryptophan (W) in the central position are also included. Cysteine (C) residues are highlighted in light grey, with the two cysteine residues that are linked to the F2 peptide indicated by an arrow above the residues. The charged amino acids closest to either side of the transmembrane region are in white type and are near the C terminus. The RSV N-linked glycosylation site in the RSV F protein is indicated with a cross.

FIG. 21 is a cartoon of the likely F protein monomer shape immediately after triggering. The horizontal line and shading at the top represent the target cell membrane and cell. The horizontal line and shading at the bottom represent the virion membrane and virion. (A) The pre-triggered F protein. (B) If triggering resulted in extension of the HR1 α-helix (6) and the remainder of the molecule remained in the same position: there would not be enough space between the virion and the cell for this fully extended form. (C) Alternate form of the triggered F protein taking into account the space constraints: the head of the molecule is pushed to one side, causing the molecule to form a “sideways V.”

FIG. 22 shows pre-triggered (A) and post-triggered (B) monomer models and pre-triggered (C) trimer model of RSV sF protein highlighting MAb resistant mutation sites. Antigenic sites are indicated, as well as their positions in different subunits (S) of the RSV trimer.

FIG. 23 shows immunoprecipitation of an RSV sF protein with MAb1243. The SC-2 sF protein, metabolically labeled with ³⁵S-Met/Cys, was treated for 1 hr at the indicated temperatures and immunoprecipitated with MAb 1243. This MAb only recognizes the pre-triggered form of the sF protein (FIG. 16). Uninfected cells (C) and virus-infected cell (V) lysates were included as negative and positive controls for the immunoprecipitation.

FIG. 24 (a-d) shows the nucleotide sequence of an optimized RSV F (optiF) gene (SEQ ID No. 6) in plasmid MP340. The optiF gene was inserted into plasmid MP319 at the SacII and XhoI sites to generate MP340. Both of these plasmids are pcDNA3.1 with the multiple cloning site replaced with convenient restriction sites (Amino acid sequences disclosed as SEQ ID NOS 35 and 36, respectively, in order of appearance).

FIG. 25 shows the sequence for the sMP340-A construct (SEQ ID NO: 37).

FIG. 26 shows the sequence for the HC-1 construct (SEQ ID NO: 38).

FIG. 27 shows the sequence for the SC-2 construct (SEQ ID NO: 39).

### DETAILED DESCRIPTION

Unless otherwise defined herein, all technical and scientific terms used herein have the same meaning as commonly understood by one of ordinary skill in the art to which this disclosure belongs. As used in the description, the singular forms “a,” “an,” and “the” are intended to include the plural forms as well, unless the context clearly indicates otherwise. All publications, patent applications, patents, and other references mentioned herein are incorporated by reference in their entirety.

Unless otherwise indicated, all numbers expressing quantities of ingredients, reaction conditions, and so forth used in the specification are to be understood as being modified in all instances by the term “about.” Accordingly, unless indicated to the contrary, the numerical parameters set forth in the following specification are approximations that may vary depending upon the desired properties sought to be obtained by the present disclosure. At the very least, and not as an attempt to limit the application of the doctrine of equivalents, each numerical parameter should be construed in light of the number of significant digits and ordinary rounding approaches.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Every numerical range given throughout this disclosure will include every narrower numerical range that falls within such broader numerical range, as if such narrower numerical ranges were all expressly written herein.

Provided herein are compositions and screening methods for identifying candidate antiviral agents. In particular, disclosed herein is a pre-triggered, fusion (F) protein of a paramyxovirus, or functional fragments thereof, which contain one or more cholesterol binding motifs in a location that is away from the transmembrane domain, referred to herein as CRAC (Cholesterol Recognition/interaction Amino acid Consensus) domains. Also provided is a computer model of the structure of the pre-triggered F protein. Compositions that directly or indirectly bind and interfere with the normal activity or binding of the pre-triggered F proteins, or the CRAC domains, are useful as antiviral agents in the treatment of paramyxovirus infections. Thus, disclosed herein are methods of screening for antiviral agents, using the pre-triggered F protein, or fragments thereof.

Paramyxovirus Fusion Mechanism:

To accomplish attachment and fusion, members of the Paramyxoviridae family express two glycoproteins, one to attach to the target cell (the attachment protein) and one to fuse the virion membrane with the target cell membrane (the fusion protein).

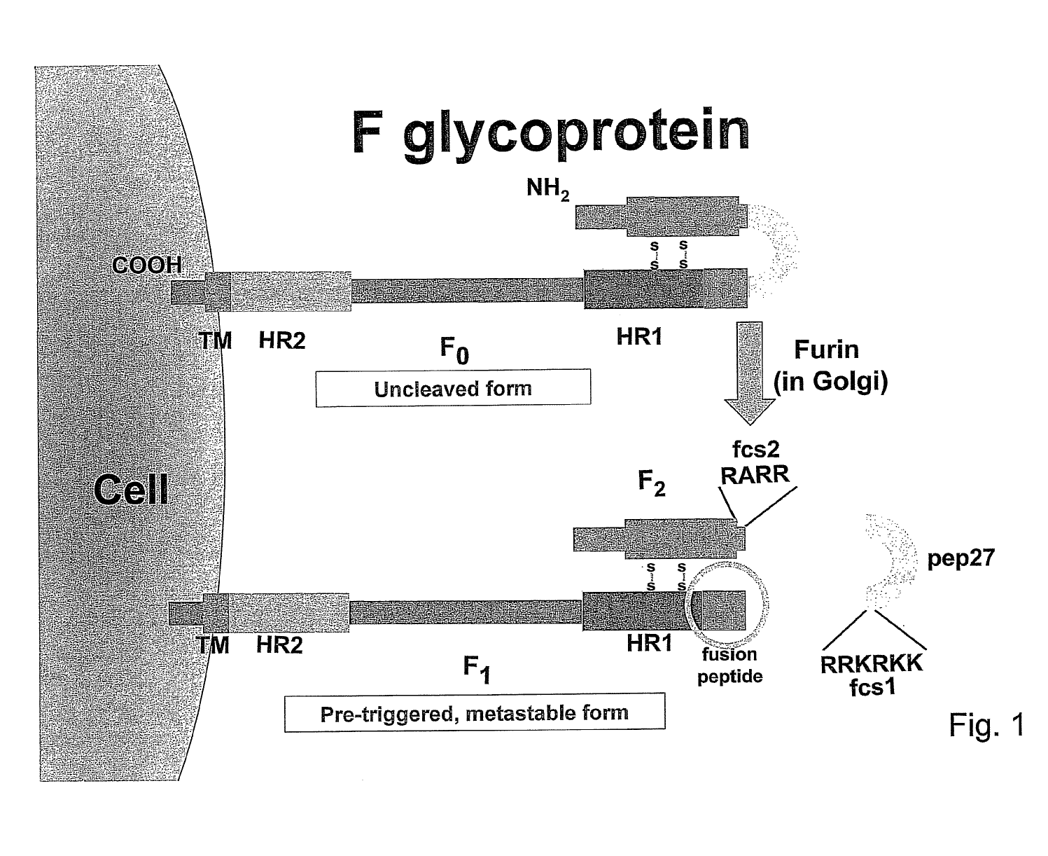

In all of these paramyxoviruses, the fusion (F) protein is a trimer composed of three copies of the F protein monomer. As the F trimer passes through the Golgi on its way to the cell surface it is cleaved by a protease to generate F2, the small N-terminal fragment, and F1, the large transmembrane fragment (FIG. 1). F2 remains covalently associated with F1 by one, or two, disulfide bonds.

In most paramyxoviruses, the conventional wisdom is that the viral attachment protein binds to its receptor, then nudges the F protein in some way that results in F protein triggering. However, RSV is unique in that its F protein is able to fuse membranes without the aid of an attachment protein, suggesting that the RSV F protein expresses both attachment and fusion activities. The RSV attachment glycoprotein (G) does enhance this process by binding the virion to target cells more efficiently, but otherwise seems to play no role in fusion.

The RSV fusion protein precursor, F0, is cleaved twice, releasing a 27 amino acid peptide “pep27” and the F1 and F2 proteins, which are covalently linked by two disulfide bonds (FIG. 1). The F1 protein is anchored in the membrane by the transmembrane (TM) domain. This cleavage activates the fusion ability of the F protein by releasing the highly hydrophobic “fusion peptide” at the N terminus of F1.

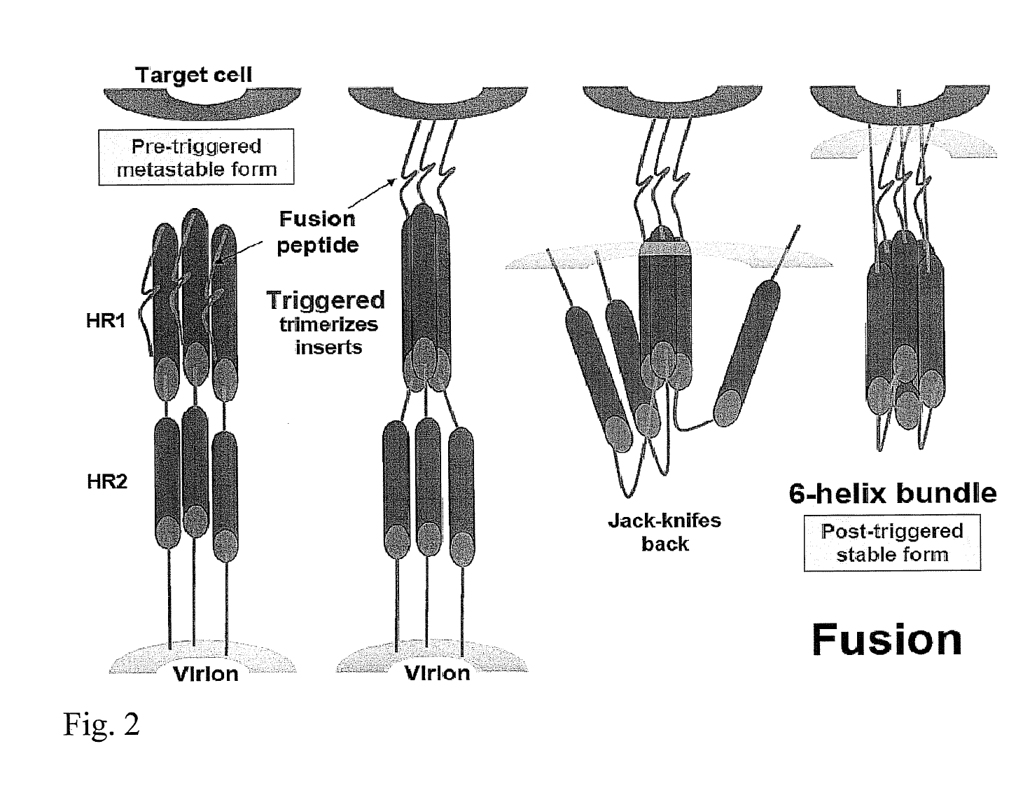

An appreciation of the movements involved in assembling the HR1 α-helix are recent and have come from the crystal structures of other paramyxoviruses. The steps in fusion initiation are shown in cartoon form in FIG. 2. At the left side of the figure, the F protein on the cell surface or in the virion is a metastable trimer with its fusion peptide tucked away. Triggering causes the fusion peptide to be exposed and to insert into the target cell membrane as HR1 forms a trimer. After insertion, the F1 protein has one end in the virion membrane and one end in the target cell membrane. The F protein then “jack-knifes” pulling the virion membrane up to the target cell membrane. The HR2α-helices lock into position in the grooves of the HR1 trimer to form the 6-helix bundle, an extremely stable structure. In so doing, the transmembrane domain linked to HR2 and the fusion peptide inserted in the plasma membrane are brought together, along with their associated membranes, initiating fusion.

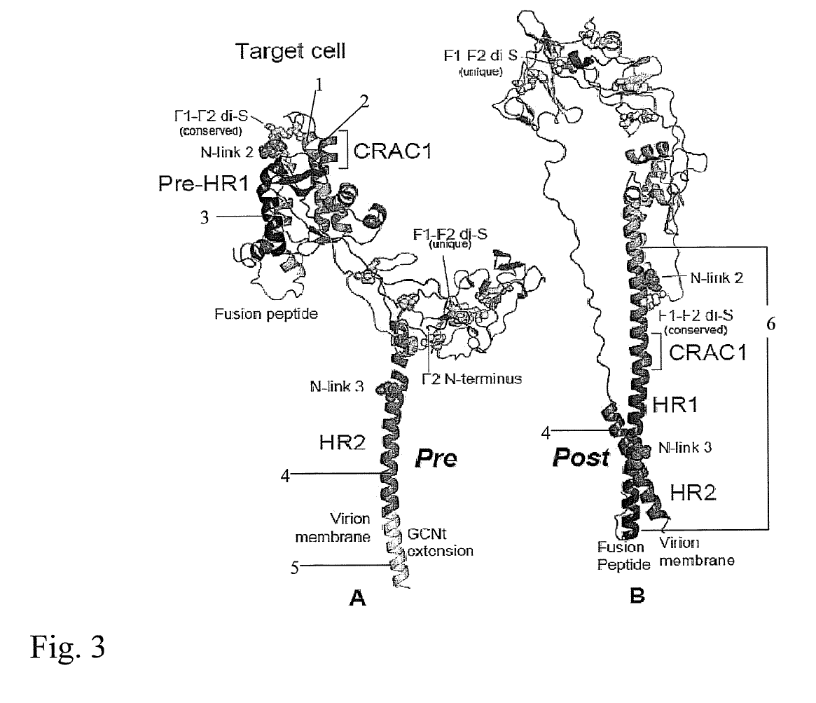

We have computer modeled the pre- and post-triggered structures of the RSV F protein, and used these models to suggest a candidate triggering domain. (EXAMPLE 1) (FIG. 3-6). To test these possibilities, we have produced pre-triggered soluble F (sF) proteins by removing the transmembrane (TM) and cytoplasmic tail (CT) domains of the RSV F protein. (EXAMPLE 2) This sF protein is similar to the active form of the F protein present on the infected cell surface or in the virion.

The model for the pre and post-triggered form of the RSV F protein is presented in FIG. 3. The differences between the structures of the pre- and post-triggered F protein indicate that it undergoes dramatic rearrangements during the triggering process. In the pre-triggered F protein, a series of three short α-helices (1, 2 and 3 in the upper left of FIG. 3A) and the regions that connect them wind back and forth over the upper left face of the molecule. In the post-triggered form, these three helices and the peptide sequences that connect them, become one long α-helix (6 in FIG. 3B).

The CRAC Domains

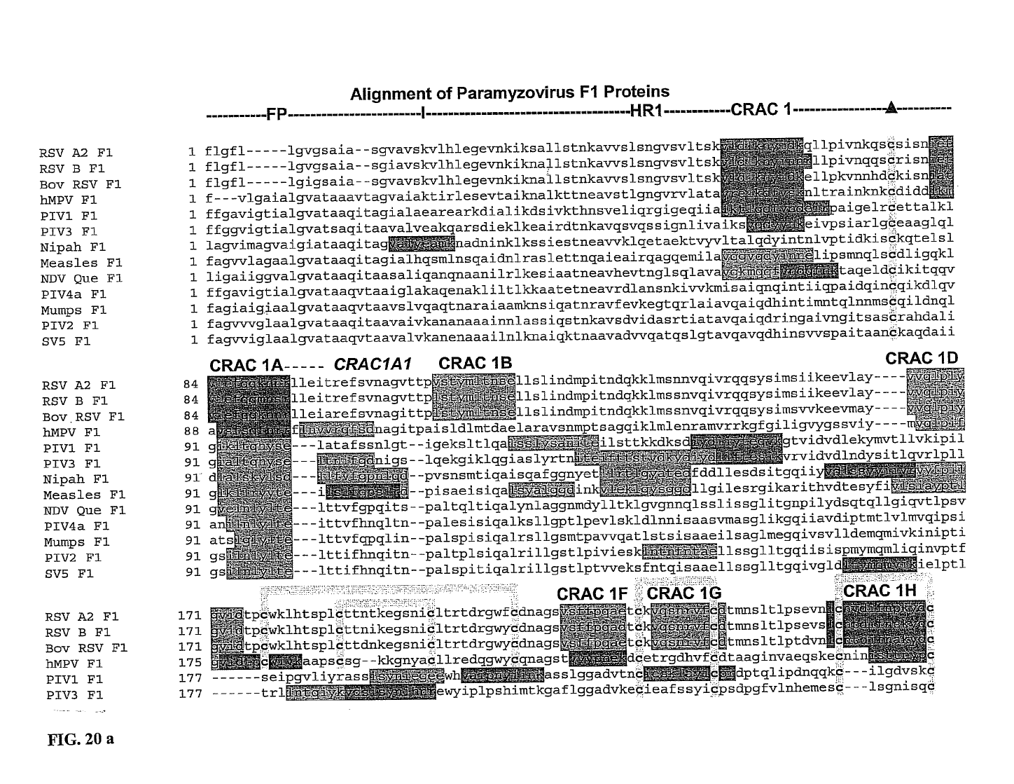

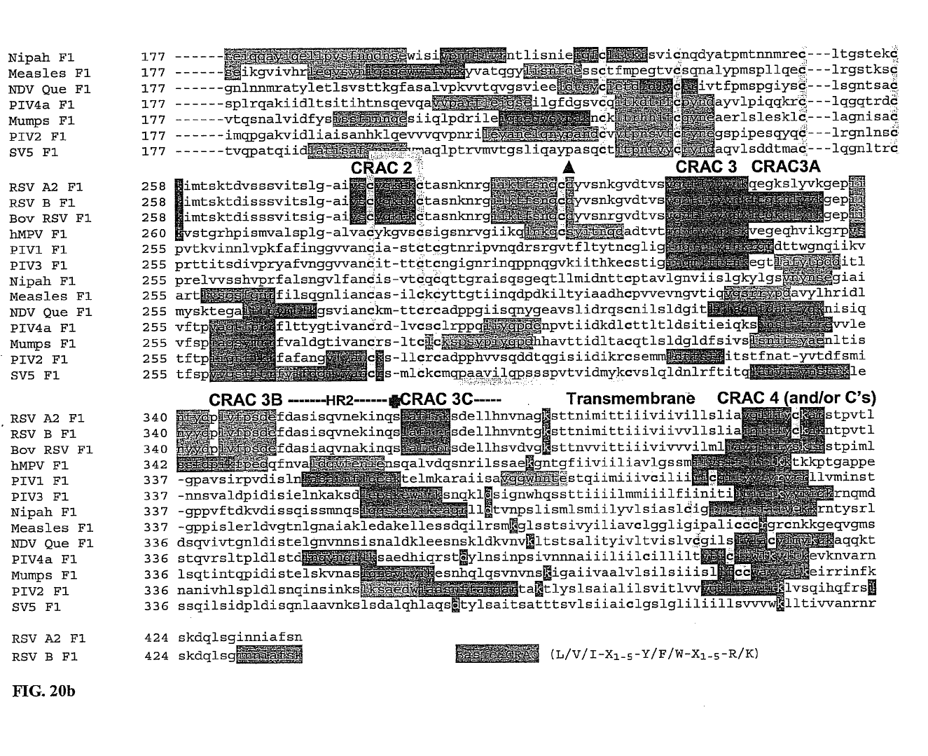

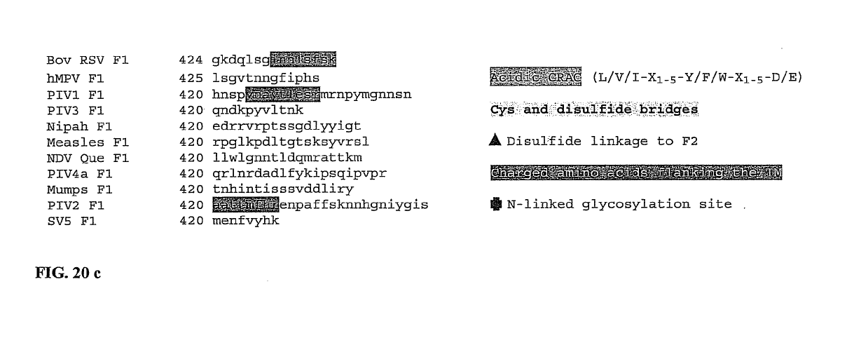

We have discovered that a cholesterol-binding protein motif (CRAC; Cholesterol Recognition/interaction Amino acid Consensus) is present near the tip of the pre-triggered F protein in a potential triggering domain (FIG. 3). The CRAC motif has been described previously as V/L-X₁₋₅-Y-X₁₋₅-R/K (Li and Papadopoulos, 1998. Endocrinology 139:4991-4997). Through our studies, we have broadened the amino acid requirement in the critical positions to: V/L/I-X₁₋₅-Y/F/W-X₁₋₅-R/K (SEQ ID No. 40) or V/L/I-X₁₋₅-Y/F/W-X₁₋₅-D/E (SEQ ID NO: 41). CRAC motifs are usually found in the juxtamembrane region of proteins that interact with cholesterol, and we have found them in the RSV F protein in juxtamembrane positions in the ectodomain (CRAC3C in FIG. 20) and the endodomain (CRAC4 in FIG. 20). However, on the RSV F protein model, there is also a CRAC motif (CRAC1) near the tip of the pre-triggered F protein structure (middle helix in the upper left of FIG. 3A), a position that is ideal for interacting with a target cell membrane. We have discovered several other CRAC motifs in the ectodomain of the RSV F protein (FIG. 20), including CRAC3 in the head region (FIG. 20). Because CRAC motifs have not been shown to function at positions in proteins that are away from membranes, such a function for these CRAC motifs is novel.

As used herein, a CRAC “motif” refers to the sequences V/L/I-X₁₋₅-Y/F/W-X₁₋₅-R/K (SEQ ID No. 40) or V/L/I-X₁₋₅-Y/F/W-X₁₋₅-D/E (SEQ ID NO: 41). A CRAC “domain” refers to a CRAC motif that is present in a position away from the virion membrane. “CRAC1 domain” refers to a CRAC motif present in the HR1 region of the F protein in a location N-terminal to the first cysteine that links the F1 to the F2 region. “CRAC3 domain” refers to a CRAC motif present in the F1 fragment, N-terminal to HR2.

Without wishing to be bound by theory, we believe that the CRAC domain(s) on the F protein interact with cholesterol in the target cell membrane and that this interaction causes triggering of the F protein, resulting in fusion.

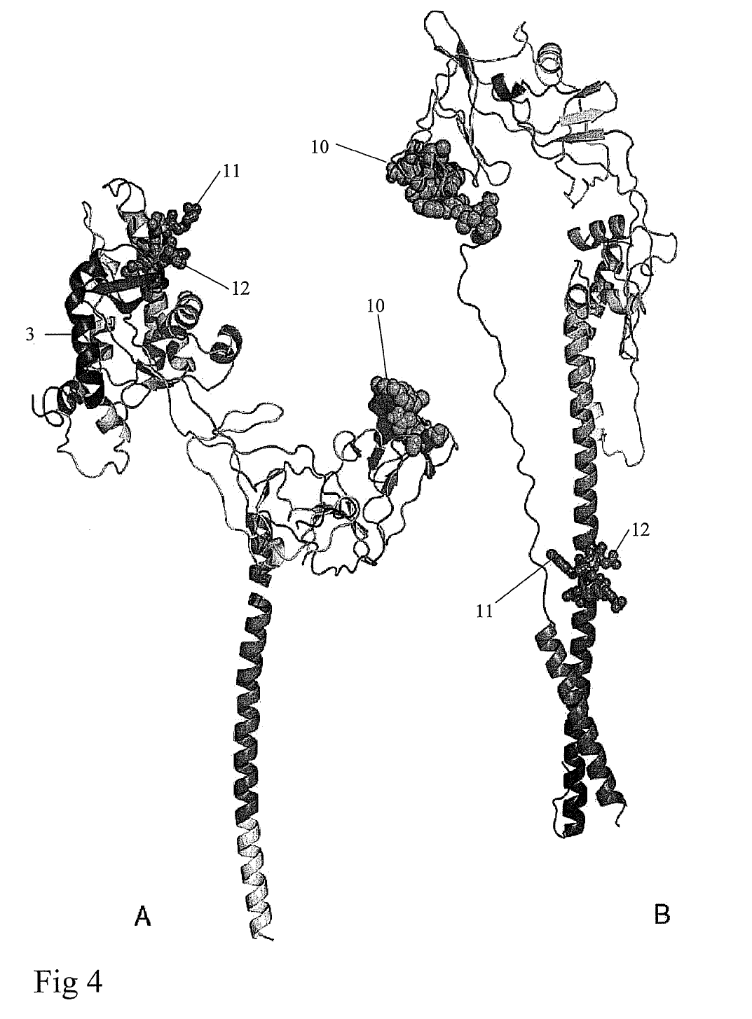

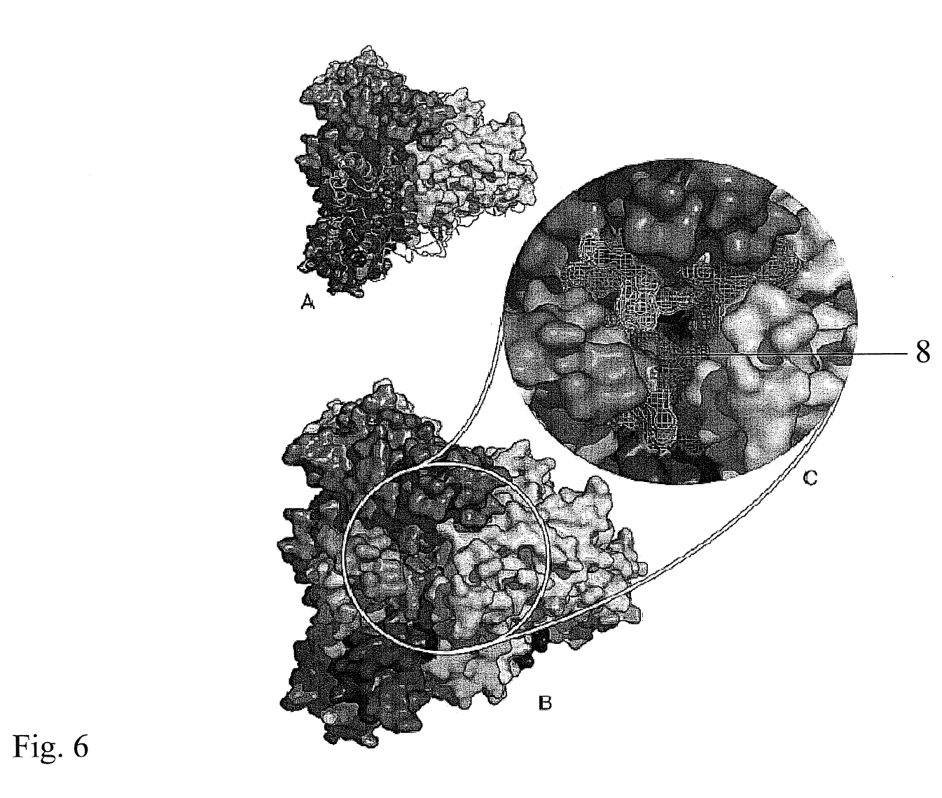

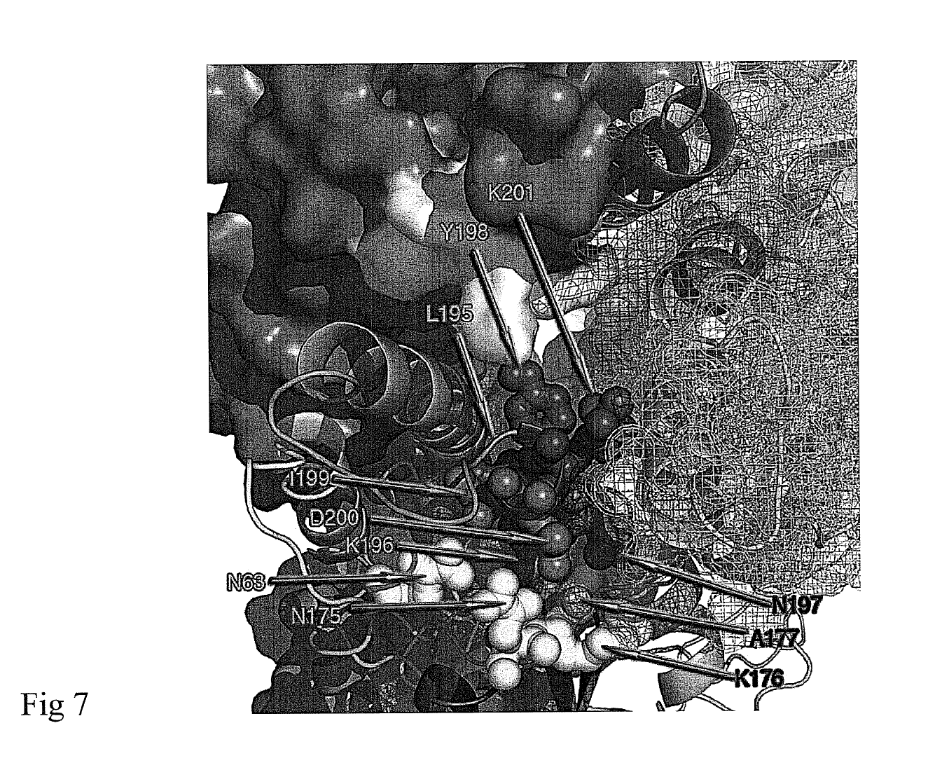

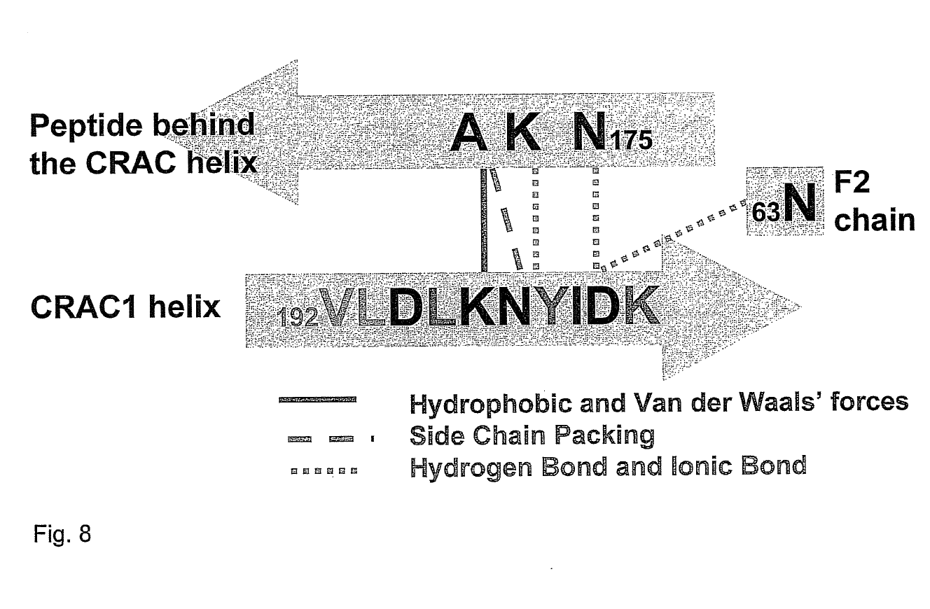

The CRAC1 domain: In the three-dimensional structure of the pre-triggered F protein, the CRAC1 domain is a short α-helix, designated α-helix 2 in FIG. 3A. Consistent with our explanation, the three critical cholesterol-binding amino acid residues in the CRAC1 domain are all on the same side of the CRAC helix and are surface exposed in the F protein monomer (FIG. 4A, dark gray balls (11)), a position that would allow these amino acids to interact with cholesterol. In the trimer, the three CRAC1 domains line the inside of a pocket formed between the short α-helices, referred to herein as the CRAC pocket (FIG. 6). The three critical CRAC amino acids all point inward, toward the central pore of the CRAC pocket in the crown of the head (FIG. 6A, B and C medium grey amino acids (8)), enabling each CRAC1 domain to bind one cholesterol molecule for a total of three cholesterol molecules per trimer. The netted regions in FIG. 6C illustrate the CRAC1 domain of each of the monomers in an F protein trimer. Three amino acids on the back side of the CRAC helix 2 in FIG. 3A, i.e. K196, N197, and D200 of SEQ ID NO: 1, interact with three amino acids N175, K176, and A177 of SEQ ID NO: 1, from a neighboring loop that links the CRAC helix (2 in FIG. 3A) to the next helix (3), and D200 interacts with N63 in the F2 peptide. A close-up of this region is shown in FIG. 7, where the uppermost balls (medium gray) represent the CRAC1 amino acids, the balls below them (dark gray) represent the amino acids on the back of the CRAC1 helix, and the balls below them (light gray) represent the interacting amino acids on the neighboring loop. As described above, in the post-triggered form, these three helices and the peptide sequences that connect them become one long α-helix (6 in FIG. 3B).

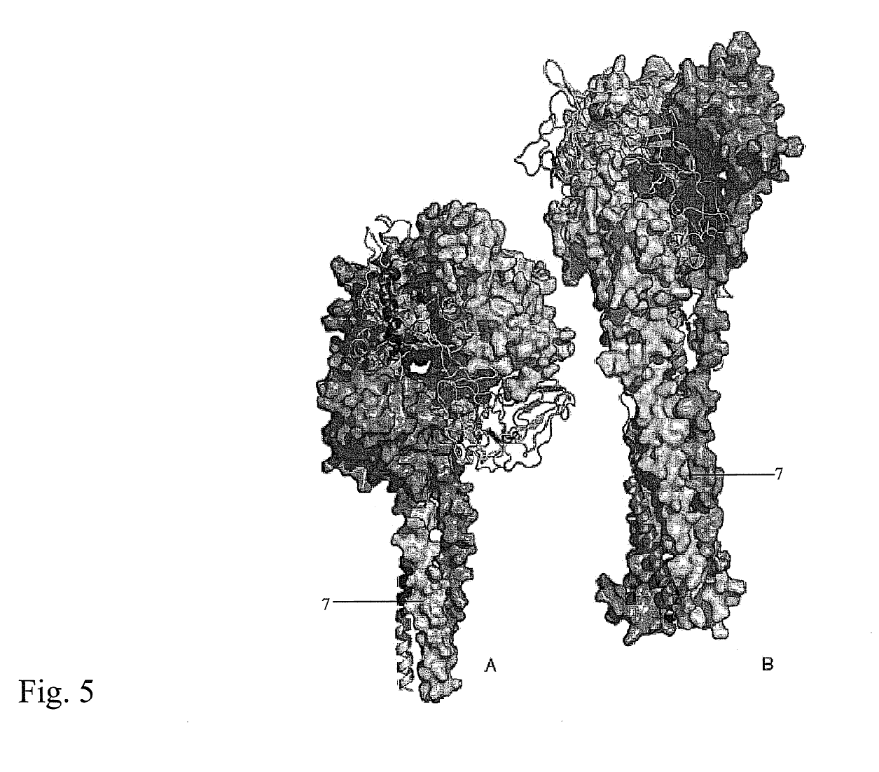

The CRAC1 helix is highlighted in ball-and-stick form in both pre- and post-triggered form in FIGS. 4A and B, respectively. The fusion peptide is the gray peptide at the end of helix (3) in the pre-triggered F protein. It is shown here in its pre-cleavage position, since the SV5 F protein structure used to model the RSV F protein was not cleaved. After cleavage, the fusion peptide is very likely inserted into the nearby hole in the side of the head (FIG. 5A).

Without wishing to be bound by theory, one of our hypotheses is that when this CRAC1 domain approaches a membrane, it is attracted by the cholesterol in the membrane, and is pulled into the membrane, initiating F protein triggering. The action of pulling the CRAC1 domain upward and onto the target cell membrane would: 1) straighten the region between the CRAC helix (2 in FIG. 3A) and helix (1), encouraging α-helix formation in this region; 2) pull the CRAC1 domain α-helix away from the stabilizing interactions with four amino acids on the neighboring peptides that interact with the backside of the CRAC helix (FIG. 7, light gray balls), thereby releasing this region to form an α-helix also; and 3) enable triggering of each of the three monomers simultaneously.

The result would be assembly of the complete long, HR1 α-helix in the post-triggered form (FIGS. 3B, 6). The three HR1 helices in the trimer would form a coiled-coil trimer, since they have a high propensity to self-assemble, even as soluble peptides. The hydrophobic fusion peptides at the end of each HR1 α-helix would be flung simultaneously against the target cell membrane during this α-helix assembly and trimerization, embedding themselves in the hydrophobic core of the membrane. This long α-helix assembly and fusion protein engagement of the target cell membrane completes the first step in membrane fusion.

Our alternative hypothesis is that cholesterol is pre-loaded in the F protein trimer crown. A trimer could pick up cholesterol molecule(s) during monomer or trimer formation, or as the trimer is transported from the endoplasmic reticulum through the Golgi to the cell surface. If cholesterol is stored in the F protein crown, as the crown approaches a target cell membrane the hydrophobic forces in the target cell membrane would pull the cholesterol molecules out of the crown and into the membrane, dragging the CRAC domains with them. This movement would initiate formation of the long α-helix (6) and insertion of the attached fusion peptide into the target cell membrane, as described above.

If cholesterol is pre-loaded in the F trimer, it would only be energetically favorable if it were held in the crown with its only hydrophilic portion, its hydroxyl group, facing the solvent (upward). The orientation of cholesterol when it is associated with a CRAC domain is known. The position of the CRAC1 domain in the crown would indeed hold cholesterol with its hydroxyl group facing upward. To the best of our knowledge, the presence of a non-membrane associated lipid within a viral protein, as suggested here, and the function of the lipid in activating a fusion protein, as discussed herein, has not been previously reported.

The CRAC1 domain is conserved among several paramyxoviruses. It is found in all pneumovirus subfamily members, including human RSV, bovine RSV, and human metapneumovirus (FIG. 20), if phenylalanine (F) is substituted for the central tyrosine (Y) in the CRAC motif. This conservation among other similar viruses confirms our finding that CRAC1 is important for the F protein to perform its fusion function. The substitution of phenylalanine for tyrosine is predictable since this is a conservative amino acid change: both amino acids contain phenyl ring.

The CRAC3 domain: The post-triggered form of the F protein contains the signature 6-helix bundle (FIGS. 3B and 4B). The second step in fusion must, therefore, be to bring the HR2α-helices to the long HR1 helix that is now a trimer (monomer is shown in FIG. 3B). In the virion, the HR2 helices are attached to the virion membrane via the transmembrane domain. After the first triggering step of fusion, described above, the trimer of HR1 helices are attached to the target cell membrane via the fusion protein. As the 3 HR2 helices lock into the grooves along the HR1 trimer (FIG. 4B), the membranes in which they are embedded are forced to mix, initiating membrane fusion.

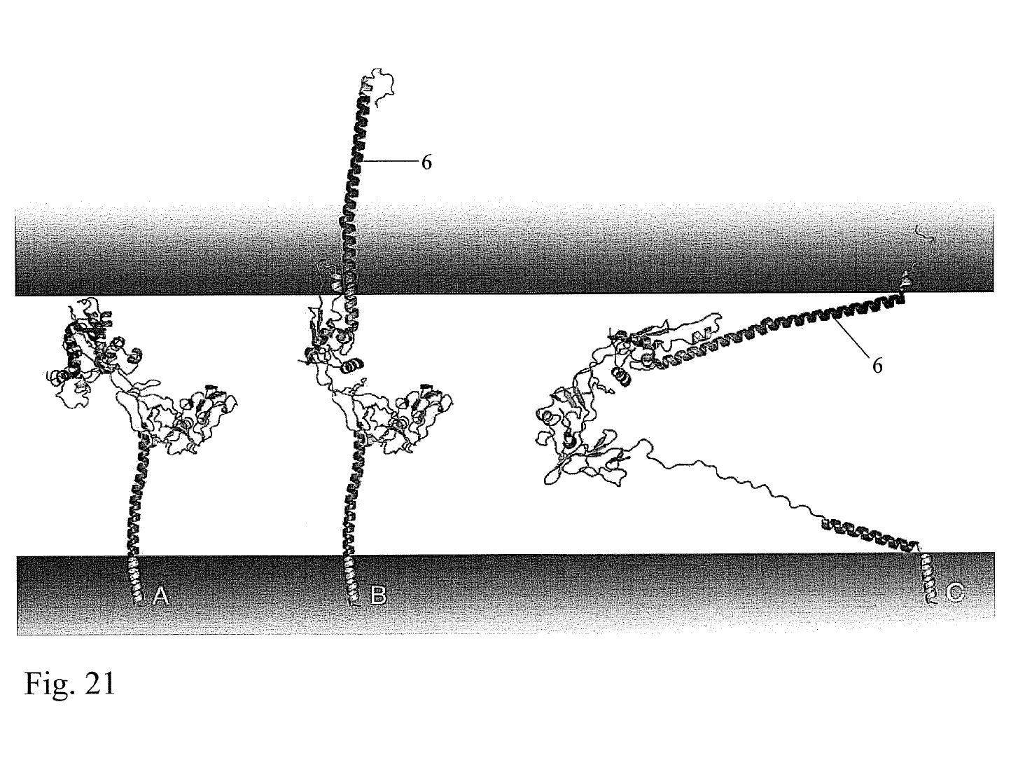

The forces that bring the 6-helix bundle together are completely unknown. Formation of the long HR1 helix more than doubles the length of the pre-triggered F protein, making it much too long to fit between the virion and the cell (FIG. 21B, where the cell is at the top and the virion is at the bottom). Since both the cell and the virion would be relatively immobile during the rapid formation of HR1, the central portion of the molecule would have to be driven sideways, unwinding and stretching out the flexible peptide that attaches the head to the HR2 helix. The result would be a “<” shaped molecule (FIG. 21C). The positioning of all 6 helices on one side of the F protein head is a requirement for 6-helix bundle formation, but has not been previously addressed.

There is no obvious “motor” in the head of the trimer (top of the post-triggered form, FIG. 3B) to drive the HR2 helices to interact with the HR1 trimer. And even if there were a motor in the head, the connection between the head of the post-triggered form and the HR2 helix lacks any rigid structure, making it impossible to drive the HR2 helix toward the HR1 helix trimer. Perhaps the virion is buffeted closer and further away from the cell membrane by Brownian motion until finally the HR2 helices come in contact with the HR1 trimer helices and each locks in to form the 6-helix bundle. With only a single connection with each membrane, this process would be very inefficient, especially considering that the flexible HR2 side of the molecule cannot maintain its “head to helix” distance to match the HR1 side. Random Brownian motion would seem unlikely to be the mechanism to line up the HR1 and HR2 helices for the required 6-helix bundle formation.

We have identified several other CRAC domains, including CRAC3 in the head region of the F protein (light gray balls (10) in FIG. 4B). CRAC3 is on the same side of the post-triggered molecule head as the HR2 helix. Without wishing to be bound by theory, it is our hypothesis that CRAC3 provides a second contact point for this side of the molecule, attaching to cholesterol in the virion membrane. Such a second contact point would stabilize this side of the molecule and hold it in a stretched out position, keeping it in the proper lateral position to find the HR1 helix trimer and lock in place.

We believe that the CRAC1 domain is a membrane contact point for the HR1 helix, enabling it to bind to the target cell membrane at a second point, the first being the fusion peptide anchored in the target cell membrane. Since the HR1 helix is rigid and long, two contact points, one at the end and one near the middle would keep this half of the protein parallel with the target cell membrane, preventing the virion from moving further from the cell. If both halves of the F protein are forced to lie parallel to the membranes into which they are inserted, the two membranes would be forced together, allowing contact between the helices and formation of the 6-helix bundle. While this hypothesis may require Brownian motion, it adds direction from the F protein in the form of additional contacts with each of the membranes that should enable the 6-helix bundle to line up and lock in much more rapidly. If both sides of the molecule are attached to the membranes at two (or more) points, the membrane curvature would be very sharp where the transmembrane and fusion peptides are brought together (at the ends of the red and blue helices, respectively) enhancing the likelihood of initiating fusion pore formation and subsequent membrane fusion.

Consistent with the hypothesis that these CRAC domains interact with the cell or virion membrane, we showed that both the CRAC1 and CRAC3 domains face outward, making such interactions possible.

We have also found that the CRAC3 domain of one F protein monomer cradles the fusion peptide of the next monomer in the pre-triggered trimer form. Cholesterol might be included in this complex. Whether or not it is, a compound that is capable of binding to the CRAC3 domain will displace the fusion peptide and cause the F protein to trigger pre-maturely. A compound that is capable of binding to the CRAC3 domain would also prevent the CRAC3 domain from forming the second contact to guide the HR2 a-helix to the HR1 trimer of helices and would prevent fusion in that way.

Other CRAC domains: As can be seen from FIG. 20, the F protein contains other CRAC domains that are conserved in all (CRAC1A) or nearly all (CRAC3/3A) of the paramyxovirus F proteins examined, suggesting that they also play a role in fusion. Others are scattered throughout the F proteins. The conserved CRAC domains, and some of the other non-conserved CRAC domains can make additional contacts with the viral or target cell membranes to enhance the fusion process. Therefore, these CRAC domains are also targets for antiviral agents. For example, a compound that can block all CRAC domain contacts with cholesterol would result in an antiviral that could attack multiple points on the F protein.

For all the reasons stated above, a compound that blocks the activity or binding of CRAC1 or CRAC3 domains to the virion membrane would reduce the efficiency of fusion, thereby reducing infection. Similarly, a compound that blocks the interaction of other F protein CRAC domains would reduce the efficiency of bringing HR1 and HR2 together, the final step in fusion initiation, thereby reducing infection.

Since cholesterol is a natural ligand for the CRAC domain, the antiviral compound can be a cholesterol mimic and/or a cholesterol precursor or derivative.

### Embodiments

### Soluble, Pre-Triggered F Protein and Fragments Thereof

Contemplated herein is an isolated soluble fusion (sF) protein of a member of the paramyxovirus family in its pre-triggered form. The isolated sF protein includes a portion of a fusion protein that contains at least one CRAC1 domain having the sequence V/L/I-X₁₋₅-Y/F/W-X₁₋₅-R/K (SEQ ID NO: 40) or V/L/I-X₁₋₅-Y/F/W-X₁₋₅-D/E (SEQ ID NO: 41).

Members of the paramyxovirus family whose F protein's include a CRAC1 domain include: RSV (human and bovine), human metapneumovirus (hMPV), para-influenza virus 1 (PIV1), PIV3, and Newcastle disease virus (NDV).

A “soluble” F protein, as used herein, refers to a truncated fusion protein that is not membrane-bound, i.e. the F protein is released form the cell into media. Thus, the soluble F protein lacks the transmembrane (TM) and cytoplasmic tail (CT) domains. In some embodiments, the pre-triggered sF protein also lacks the pep27 region.

A “soluble F protein of a member of the paramyxovirus family that includes a CRAC1 domain” refers to any soluble fusion protein that includes a CRAC1 domain, and whose sequence is at least 80%, 85%, 90%, 91%, 92%, 93%, 94%, 95%, 96%, 97%, 98%, 99%, or 100% identical to the sequence of a truncated F protein of: human RSV, bovine RSV, hMPV, PIV1, PIV3 and NDV.

In one embodiment, the CRAC domain has the sequence VLDLKNYIDK, SEQ ID NO: 20. In another embodiment, the CRAC domain has the sequence VLDLKNYIDR, SEQ ID NO: 42. In another embodiment, the CRAC domain has the sequence VLDIKNYIDK, SEQ ID NO: 43. In another embodiment, the CRAC domain has the sequence ILDLKNYIDK, SEQ ID NO: 44. In another embodiment, the CRAC domain has the sequence VLDLKNYINNR, SEQ ID NO: 45. In another embodiment, the CRAC domain has the sequence VRELKDFVSK, SEQ ID NO: 46. In another embodiment, the CRAC domain has the sequence LKTLQDFVNDEIR, SEQ ID NO: 47. In another embodiment, the CRAC domain has the sequence VQDYVNK, SEQ ID NO: 48. In another embodiment, the CRAC domain has the sequence VNDQFNK, SEQ ID NO: 49.

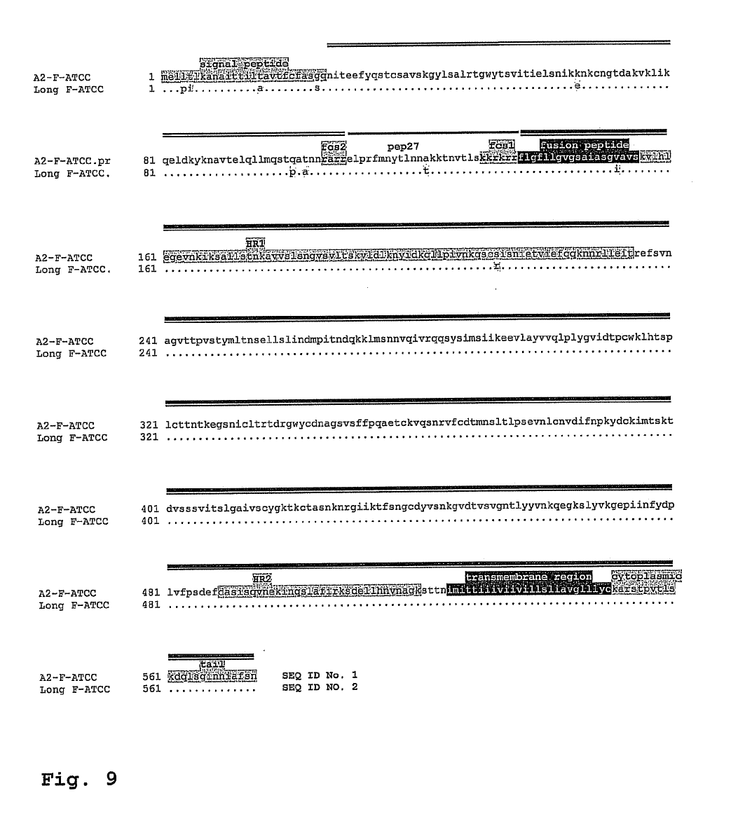

SEQ ID NO: 1 represents the full length amino acid sequence of the A2 strain RSV F protein (FIG. 9). The full length RSV F protein may be divided into several structurally and functionally distinct regions, with reference to SEQ ID No 1. The signal peptide is from amino acid 1-25. The F2 fragment is from amino acids 26 to 109, with the fcs2 cleavage site located at amino acids 106 to 109. The pep27 peptide, which is cleaved away during in vivo processing, is from amino acid 110 to 136, with the fcs1 cleavage site located at amino acids 131-136. The F1 fragment is from amino acid 137 to 574, with the fusion peptide located at amino acids 137 to 155, the heptad repeat HR1 is located at amino acids 156 to 234, the heptad repeat HR2 is located at amino acids 489 to 514, the transmembrane region is at amino acids 521 to 550, and the cytoplasmic tail is located at amino acids 551 to 574.

In one embodiment, each monomer of the sF protein trimer includes an amino acid sequence that is at least 80%, 85%, 90%, 91%, 92%, 93%, 94%, 95%, 96%, 97%, 98%, 99%, or 100% identical to: amino acid 27-109 and 137-522 of SEQ ID NO: 1. In another embodiment, each monomer of the sF protein trimer includes an amino acid sequence that is at least 80%, 85%, 90%, 91%, 92%, 93%, 94%, 95%, 96%, 97%, 98%, 99%, or 100% identical to amino acid 27-522 of SEQ ID NO: 1. Amino acids 523 and 524 of SEQ ID NO: 1 may be deleted or changed to other amino acids. Therefore, in another embodiment, the sF protein comprises amino acid 27-524 of SEQ ID NO: 1.

The signal peptide (amino acids 1-25 in SEQ ID NO: 1) is used to start the translocation of the protein across the ER membrane during synthesis. In some embodiments, the constructs that are used to prepare a pre-triggered sF protein also include a sequence encoding a signal peptide. In one example, the signal peptide encoded by the construct comprises amino acids 1-25 in SEQ ID NO: 1. In other examples, the signal peptide encoding sequence may be exchanged for other signal peptide encoding sequences that are capable of starting the in vivo translocation of the protein across the ER membrane during synthesis. Examples of other suitable signal peptides include, but are not limited to, the signal peptide of another polypeptide naturally expressed by the expression host cell, the Campath leader sequence (Page, M. J. et al., BioTechnology 9:64-68 (1991)), the signal peptide and the pre-pro region of the alkaline extracellular protease (AEP) (Nicaud et al. 1989. J Biotechnol. 12: 285-298), secretion signal of the extracellular lipase encoded by the LIP2 gene (Pignede et al., 2000 Appi Environ. Microbiol. 66: 3283-3289.), the 22 amino acid signal peptide of the endoglucanase I coding sequence from T. reesei (Park, C. S., (1997). J. Biol. Chem. 272: 6876-6881), the rice ct-amylase signal peptide (Chen et al., 2004 Plant Physiol. 135: 1363-1377), the signal peptide for pre-proinsulin, immunoglobulin kappa chain, or any type I glycoprotein or protein that is normally secreted from mammalian cells. A type I glycoprotein is a protein that has its N terminus outside the cell plasma membrane and its C terminus inside.

In some embodiments, the sF protein is also fused to a detection tag that is useful for identification or purification. Examples of commonly used detection tags include, but are not limited to, a maltose-binding protein (MBP), glutathione S-transferase (GST), tandem affinity purification (TAP) tag, calcium modulating protein (calmodulin) tag, covalent yet dissociable (CYD) NorpD peptide, Strep II, FLAG tag, heavy chain of protein C(HPC) peptide tag, green fluorescent protein (GFP), metal affinity tag (MAT), HA (hemagglutinin) tag, 6HIS tag (SEQ ID NO: 21), myc tag, and/or herpes simplex virus (HSV) tag. In some embodiments, the tag is a FLAG tag or a 6HIS tag (SEQ ID NO: 21). In one embodiment, the protein comprised both a FLAG tag and a 6HIS tag (SEQ ID NO: 21). In some embodiments, the polypeptide further comprises a cleavage domain to facilitate the removal of the tag from the polypeptide, for example, after isolation of the protein. In some embodiments, the tag is fused to the C terminus of the sF protein. The tag or tags can also be placed at the N terminus of the F2 protein, C terminal to the signal peptide. For example, we have placed a 6HIS tag (SEQ ID NO: 21) in this position and rescued fully functional RSV from cDNA that contains this tag on the F protein, indicating that the tag did not negatively impact production or function of the F protein. The tag or tags can also be placed in other positions in the protein as additional or replacement amino acids, generally in external loops of the protein where the amino acids comprising the tag would not affect protein folding or function.

In some embodiments, the sF protein contains a C terminal “clamp” to hold the C terminus of the protein in position. The clamp holds the C termini of the three monomers in the molecule together, preventing them from separating or moving upward and triggering the molecule. In one example, the C terminal clamp is a trimerization domain, such as GCNt. The sF protein with the GCNt clamp that we produced, sMP340-A, is secreted efficiently from transfected cells but it is not recognized efficiently by MAbs against the F protein, may be partially aggregated, and is not triggered by treatment at 50 C for one hour. Minor modifications to this construct, however, will likely result in a pre-triggered sF protein. Those modifications include removal of the glycine that we had inserted between the sF protein C terminus and the GCNt clamp to add flexibility, removal of residues or insertion of residues such as alanine, that will not disturb the helical nature of this region but which can bring the HR2 helix and the GCNt helix into phase with each other. In another example, the clamp contains a trimerization domain comprising two cysteines that will covalently link the three monomers. In this example, two amino acids at or near the C terminus of the HR2 helix in each soluble F protein monomer are replaced with two cysteines. The cysteines are either consecutive or have one or more amino acids separating them. The 6 cysteines in the trimer will form 3 disulfide bonds, linking the C termini of the three monomers.

For example, the sF protein stabilized at its C terminus by either the addition of a GCNt clamp or cysteines are useful tools for assessing the first step of triggering, i.e., unfolding of the HR1 domain, without the second step of forming the 6-helix bundle. Because the HR2 helices are linked in this protein, they will not be able to fit into the grooves provided by the HR1 trimer to produce the 6-helix bundle. On the other hand, the sF protein without the cysteines will be able to perform both unfolding of the HR1 domain and formation of the 6-helix bundle because its C terminus is not cross-linked to the other monomers in the trimer. So, the clamp or the Cys linkage would probably stabilize the sF protein making it easier to store and to use since more of it would remain in the pre-triggered form. For example, SC-2 begins to decay as soon as it is made, with a t½ of about 3 weeks.

In addition, several strategies are available to produce and maintain and/or stabilize the isolated sF protein or its fragments in the pre-triggered state, i.e. to prevent the triggering of the protein during synthesis and storage. These strategies include: using freshly prepared sF protein in the assays described below; storing the sF protein at 4° C. under which conditions the pre-triggered sF protein slowly triggers, with a half-life of approximately 3 weeks; snap freezing the isolated sF protein on dry ice or liquid nitrogen; and thawing at 37° C. To maintain the sF protein in its pre-triggered form, it is desirable to avoid harsh treatments or treatments which allow triggering to occur. For example, freezing the protein slowly by placing it in a −20° C. freezer or maintaining it at 37° C. or higher for any appreciable amount of time may allow the protein to trigger. In another example, extremes of pH, such as the low pH needed to remove an isolated sF protein from an antibody affinity column should likely be avoided. As described above, the sF protein may also be physically stabilized by adding a GCNt segment to clamp the C terminus, or by adding cysteines that will cross-link the trimer C termini.

Any isolated sF protein that has less than 100% identity with the reference amino acid sequence of the F protein (e.g. SEQ ID NO: 1) is a variant protein. A variant protein has an altered sequence in which one or more of the amino acids in the reference sequence, other than the amino acids that constitute the CRAC domains, is deleted or substituted, or one or more amino acids are inserted into the sequence of the reference amino acid sequence (as described above). A variant can have any combination of deletions, substitutions, or insertions.

With regard to amino acid substitutions, a variety of amino acid substitutions can be made. As used herein, amino acids generally can be grouped as follows: (1) amino acids with non-polar or hydrophobic side groups (A, V, L, I, P, F, W, and M); (2) amino acids with uncharged polar side groups (G, S, T, C, Y, N, and Q); (3) polar acidic amino acids, negatively charged at pH 6.0-7.0 (D and E); and (4) polar basic amino acids, positively charged at pH 6.0-7.0 (K, R, and H). Generally, “conservative” substitutions, i.e., those in which an amino acid from one group is replaced with an amino acid from the same group, can be made without an expectation of impact on activity. Further, some non-conservative substitutions may also be made without affecting activity. Those of ordinary skill in the art will understand what substitutions can be made without impacting activity.

It should be noted that proteins disclosed herein may also comprise amino acids linked to either end, or both. These additional sequences may facilitate expression, purification, identification, solubility, membrane transport, stability, activity, localization, toxicity, and/or specificity of the resulting polypeptide, or may be added for some other reason. The proteins disclosed herein may be linked directly or via a spacer sequence. The spacer sequence may or may not comprise a protease recognition site to allow for the removal of amino acids.

It should be further noted that proteins disclosed herein may also comprise non-amino acid tags linked anywhere along the protein. These additional non-amino acid tags may facilitate expression, purification, identification, solubility, membrane transport, stability, activity, localization, toxicity, and/or specificity of the resulting polypeptide, or it may be added for some other reason. The proteins disclosed herein may be linked directly or via a spacer to the non-amino acid tag. Examples of non-amino acid tags include, but are not limited to, biotin, carbohydrate moieties, lipid moieties, fluorescence groups, and/or quenching groups. The proteins disclosed herein may or may not require chemical, biological, or some other type of modification in order to facilitate linkage to additional groups.

Also provided herein are functional fragments of the isolated sF protein. The terms “fragment” and “functional fragment” are used interchangeably and refer to an isolated peptide that is a truncated from of the pre-triggered soluble F protein and that can successfully function in any of the screening tests described below. The functional fragments comprise some or most of the amino acid sequence of the pre-triggered sF protein, and include a CRAC1 domain. Several regions of the sF protein may be deleted or modified to form a functional fragment.

In one embodiment, the CRAC domain has the sequence VLDLKNYIDK, SEQ ID NO: 20. In another embodiment, the CRAC domain has the sequence VLDLKNYIDR, SEQ ID NO: 42. In another embodiment, the CRAC domain has the sequence VLDIKNYIDK, SEQ ID NO: 43. In another embodiment, the CRAC domain has the sequence ILDLKNYIDK, SEQ ID NO: 44. In another embodiment, the CRAC domain has the sequence VLDLKNYINNR, SEQ ID NO: 45. In another embodiment, the CRAC domain has the sequence VRELKDFVSK, SEQ ID NO: 46. In another embodiment, the CRAC domain has the sequence LKTLQDFVNDEIR, SEQ ID NO: 47. In another embodiment, the CRAC domain has the sequence VQDYVNK, SEQ ID NO: 48. In another embodiment, the CRAC domain has the sequence VNDQFNK, SEQ ID NO: 49.

In one embodiment, the functional fragment is a fragment of RSV F protein. In some embodiments of the RSV functional fragment, all or some of the amino acids N terminal to Cys37 are deleted or replaced.

In other embodiments, all or a portion of the amino acid sequence between and including Asn70 and S155 is removed or replaced. In some other embodiments, all or a portion of the fusion peptide (a.a. 137-155) is removed. In yet other embodiments, all or a portion of the amino acid sequence from Asn70 and R136 is removed or replaced. In some embodiments, pep27 (a.a. 110-136) is removed or replaced with alanines and glycines without destroying the function of the F protein.

In some embodiments, part, or all, of the HR2 region is removed. In some embodiments, the C terminus is truncated, up to and including D440. In some embodiments, a tryptophan or phenylalanine replaces the tyrosine Y198, an arginine replaces R201, an isoleucine, leucine or valine replaces V192, L193, or L195.

In some embodiments, cysteines C37, C69, C212 and C439 link the F1 and F2 fragments together. In other embodiments, these cysteines are replaced by amino acids that interact in a non-covalent manner to hold the F1 and F2 fragments together. Although no residues substitute for cysteines in terms of creating covalent cross-linked bonds, there are many hydrogen-bonding/salt-bridge networks, and hydrophobic-packing networks that can functionally substitute for the stability provided by cysteine residue disulfide bonds. For instance, cysteine residues can coordinate Zinc, rather than link covalently, as in the lid domain of adenylate kinase. The structure of the adenylate kinase lid domain is stabilized by either 4 cysteine residues which coordinate a zinc ion rather than covalently link through disulfide bonds, or by a variable set of 6 residues that engage in salt-bridges, polar interactions, and hydrogen bonding. These 4 cysteine residues can be replaced by several combinations of charged/polar residues at these 6 partially overlapping positions on the structure. Another example would be a leucine zipper that is used in many proteins as a mechanism to dimerize. Another example is found where there is a valine-alanine interaction that substitutes for a disulfide bonded cysteine pair, e.g. in the PIV5 structure (387-410 in the 2B9B PDB structure).

In one embodiment, the fragment is a “dimer peptide” comprising two peptides, each of which comprise, respectively, an amino acid sequence that is at least 90%, 95%, 96%, 97%, 98%, 99% or 100% identical to amino acids 37-69 (F2 fragment) and 156-440 (F1 fragment, including the CRAC1 domain) of SEQ ID NO: 1, linked together.

In other embodiments, any number of amino acids can be added to either end of the dimer peptide. In some embodiments, the additional one or more amino acids that are added to the “dimer peptide” are identical to, or are conservative substitutions for, the amino acids found between amino acids 26-36, 70-155 and/or 441-522 of SEQ ID NO: 1.

Different fragments may be used in different screening methods, as described below.

Method of producing the pre-triggered, soluble F protein and fragments thereof.

Also provided herein are methods of producing the isolated pre-triggered, soluble (s) F protein of paramyxoviruses. In general, any suitable method known in the art for the production of glycoproteins can be used for the purpose of producing the pre-triggered sF protein and fragments thereof.

In some embodiments, the method comprises using a nucleic acid molecule (e.g. RNA) encoding the truncated F protein in a cell-free translation system to prepare the soluble F protein, or functional fragments thereof. Alternatively, a nucleic acid molecule (e.g. DNA) encoding the truncated F protein, or functional fragments thereof, is introduced into an expression vector and used to transform cells. In the expression vector, the sequence which encodes the truncated F protein is operatively linked to an expression control sequence, i.e., a promoter, which directs mRNA synthesis.

Suitable expression vectors include for example chromosomal, nonchromosomal and synthetic DNA sequences, e.g., derivatives of SV40, bacterial plasmids, phage DNAs; yeast plasmids, vectors derived from combinations of plasmids and phage DNAs, viral DNA such as vaccinia, adenovirus, fowl pox virus, and pseudorabies. The DNA sequence is introduced into the expression vector by conventional procedures.

Sequences of the paramyxovirus F proteins are publicly available. For example, in some embodiments, the F protein has the sequence SEQ ID NO: 1. Other examples of RSV F protein sequences are presented in Table 1.

TABLE 1
Accession numbers and description of RSV F protein sequences

```
  1: U39662 Human respiratory syncytial virus S2, complete genome
gi|1912287|gb|U39662.1|HRU39662[1912287]
  2: DQ885231 Human respiratory syncytial virus strain A fusion protein (F) gene, complete
cds
gi|113472469|gb|DQ885231.1|[113472469]
  3: NC_001781 Human respiratory syncytial virus, complete genome
gi|9629198|ref|NC_001781.1|[9629198]
  4: D00334 Human respiratory syncytial virus gene for fusion protein precursor, complete
cds
gi|222564|dbj|D00334.1|RSHFB[222564]
  5: AY911262 Human respiratory syncytial virus strain ATCC VR-26, complete genome
gi|60549163|gb|AY911262.1|[60549163]
  6: D00151 Human respiratory syncytial virus genes for fusion protein and 22K protein,
complete cds
gi|222548|dbj|D00151.1|RSH22K[222548]
  7: Z26524 Human respiratory syncytial virus F gene for fusion protein
gi|403378|emb|Z26524.1|[403378]
  8: X02221 Human respiratory syncytial virus (A2) mRNA for fusion glycoprotein Fo
gi|61210|emb|X02221.1|[61210]
  9: D00396 Human respiratory syncytial virus (subgroup B/strain 18537) gene for 22K
protein, partial cds
gi|60683820|dbj|D00396.2|[60683820]
10: AF035006 Human respiratory syncytial virus, recombinant mutant rA2cp, complete
genome
gi|3089371|gb|AF035006.1|[3089371]
11: U50363 Human respiratory syncytial virus, mutant cpts-248, complete genome
gi|2627309|gb|U50363.1|HRU50363[2627309]
12: U50362 Human respiratory syncytial virus, mutant cp-RSV, complete genome
gi|2627296|gb|U50362.1|HRU50362[2627296]
13: AY330616 Human respiratory syncytial virus strain Long fusion protein precursor,
gene, complete cds
gi|37674753|gb|AY330616.1|[37674753]
14: AY330615 Human respiratory syncytial virus strain Long clone T444C fusion protein
precursor, gene, complete cds
gi|37674751|gb|AY330615.1|[37674751]
15: AY330614 Human respiratory syncytial virus strain Long clone T433A fusion protein
precursor, gene, complete cds
gi|37674749|gb|AY330614.1|[37674749]
16: AY330613 Human respiratory syncytial virus strain Long clone T1480G fusion protein
precursor, gene, complete cds
gi|37674747|gb|AY330613.1|[37674747]
17: AY330612 Human respiratory syncytial virus strain Long clone A1194G fusion protein
precursor, gene, complete cds
gi|37674745|gb|AY330612.1|[37674745]
18: AY330611 Human respiratory syncytial virus strain Long clone A1188G fusion protein
precursor, gene, complete cds
gi|37674743|gb|AY330611.1|[37674743]
19: AF512538 Human respiratory syncytial virus isolate LLC1144-115 small hydrophobic
protein (SH), attachment glycoprotein (G), and fusion glycoprotein (F) genes, complete cds
gi|21729393|gb|AF512538.2|[21729393]
20: AY114151 Human respiratory syncytial virus isolate LLC62-111 small hydrophobic
protein (SH), attachment glycoprotein (G), and fusion glycoprotein (F) mRNAs, complete cds
gi|21689584|gb|AY114151.1|[21689584]
21: AY114150 Human respiratory syncytial virus isolate LLC242-282 small hydrophobic
protein (SH), attachment glycoprotein (G), and fusion glycoprotein (F) mRNAs, complete cds
gi|21689580|gb|AY114150.1|[21689580]
22: AY114149 Human respiratory syncytial virus isolate LLC235-267 small hydrophobic
protein (SH), attachment glycoprotein (G), and fusion glycoprotein (F) mRNAs, complete cds
gi|21689576|gb|AY114149.1|[21689576]
23: AY198177 Human respiratory syncytial virus strain B65 fusion protein gene, complete
cds
gi|29290042|gb|AY198177.1|[29290042]
24: AY198175 Human respiratory syncytial virus strain E65 fusion protein gene, complete
cds
gi|29290038|gb|AY198175.1|[29290038]
25: L25351 Human respiratory syncytial virus fusion protein (F) mRNA, complete cds
gi|409060|gb|L25351.1|RSHFUSP[409060]
26: M11486 Human respiratory syncytial virus nonstructural protein (1C), nonstructural
protein (1B), major nucleocapsid (N), phosphoprotein (P), protein (M), 1A (1A), G (G),
protein (F) and envelope-associated protein (22K) gene, complete cds
gi|333925|gb|M11486.1|RSH1CE[333925]
27: AF013255 Human respiratory syncytial virus mutant cp52, complete genome
gi|2582034|gb|AF013255.1|AF013255[2582034]
28: AF013254 Human respiratory syncytial virus wildtype strain B1, complete genome
gi|2582022|gb|AF013254.1|AF013254[2582022]
29: U63644 Human respiratory syncytial virus, mutant cpts-248/404, complete genome
gi|1695254|gb|U63644.1|HRU63644[1695254]
30: U31562 Human respiratory syncytial virus, strain RSB89-6614, fusion protein (F)
mRNA, complete cds
gi|961614|gb|U31562.1|HRU31562[961614]
31: U31561 Human respiratory syncytial virus, strain RSB89-6256, fusion protein (F)
mRNA, complete cds
gi|961612|gb|U31561.1|HRU31561[961612]
32: U31560 Human respiratory syncytial virus, strain RSB89-1734, fusion protein (F)
mRNA, complete cds
gi|961610|gb|U31560.1|HRU31560[961610]
33: U31559 Human respiratory syncytial virus, strain RSB89-5857, fusion protein (F)
mRNA, complete cds
gi|961608|gb|U31559.1|HRU31559[961608]
34: U31558 Human respiratory syncytial virus, strain RSB89-6190, fusion protein (F)
mRNA, complete cds
gi|961606|gb|U31558.1|HRU31558[961606]
35: M74568 Human respiratory syncytial virus nonstructural protein 1, nonstructural
protein 2, nucleocapsid protein, phosphoprotein, matrix protein, small hydrophobic protein,
glycoprotein, fusion glycoprotein, 22K/M2 protein and L protein mRNA, complete cds
gi|333959|gb|M74568.1|RSHSEQ[333959]
36: M22643 Human respiratory syncytial virus fusion (F) protein mRNA, complete cds
gi|333938|gb|M22643.1|RSHF1[333938]
```

Promoters vary in their “strength” (i.e. their ability to promote transcription). For the purposes of expressing a cloned gene, it is desirable to use strong promoters in order to obtain a high level of transcription and, hence, expression of the gene. Depending upon the host cell system utilized, any one of a number of suitable promoters may be used. For instance, when cloning in E. coli, its bacteriophages, or plasmids, promoters such as the T7 phage promoter, lac promoter, trp promoter, recA promoter, ribosomal RNA promoter, the PR and PL promoters of coliphage lambda and others, including but not limited, to lacUV5, ompF, bla, lpp, and the like, may be used to direct high levels of transcription of adjacent DNA segments. Additionally, a hybrid trp-lacUV5 (tac) promoter or other E. coli promoters produced by recombinant DNA or other synthetic DNA techniques may be used to provide for transcription of the inserted gene. Examples of constitutive promoters for use in mammalian cells include the RSV promoter derived from Rous sarcoma virus, the CMV promoter derived from cytomegalovirus, β-actin and other actin promoters, and the EF1α promoter derived from the cellular elongation factor 1α gene. Other examples of some constitutive promoters that are widely used for inducing expression of transgenes include the nopoline synthase (NOS) gene promoter, from those derived from any of the several actin genes, which are known to be expressed in most cells types, and the ubiquitin promoter, which is a gene product known to accumulate in many cell types. Other promoters include the SV40 promoter, or the or murine leukemia virus long terminal repeat (LTR) promoters.

Examples of host cells include a variety of eukaryotic cells. Suitable mammalian cells for use in the present invention include, but are not limited to Chinese hamster ovary (CHO) cells, Vero (African kidney), baby hamster kidney (BHK) cells, human HeLa cells, A549 (human type II pneumocyte), HEp-2 (human neck epithelial) cells, monkey COS-1 cell, human embryonic kidney 293T cells, mouse myeloma NSO and human HKB cells. Other suitable host cells include insect cell lines, including for example, Spodoptera frugiperda cells (Sf9, Sf21), Trichoplusia ni cells, and Drosophila Schneider Line 1 (SL1) cells.

In another embodiment, the method of production includes the same steps but in a cell line capable of high density growth without serum. Examples include, but are not limited to mammalian cells including HKB11 (a hybrid cell line from human embryonic kidney 293 and a human B cell line), CHO (Chinese hamster ovary cells, NS0 (mouse myeloma), and SP2/0 Ag14 (mouse myeloma).

Alternative methods include using insect or yeast cells infected by a viral vector to deliver and express the sF gene. Examples of viral vectors include, but are not limited to: Sindbis virus, adenovirus or vaccinia virus in mammalian cells, or baculovirus in insect, or mammalian, cells.

In some embodiment, the RSV sF protein gene sequence is derived by reverse transcription as cDNA and inserted into a plasmid behind a promoter such as the bacteriophage T7, SP6 or other similar promoter. The plasmid is transfected into cells along with a plasmid expressing the corresponding T7, SP6 or other polymerase, or a viral vector producing this polymerase. In these systems, the sF protein will be expressed in the cytoplasm of a cell, resulting in sF protein production and secretion.

The cDNA sequence derived from the RSV genome or mRNA cannot be inserted into a plasmid and expressed from the nucleus. Since RSV replicates in the cytoplasm, its mRNA is not exposed to the nuclear splicing and polyadenylation machinery. The RSV F protein contains 4 nuclear polyadenylation sites (Ternette, et al. 2007. Vaccine. 2007 25(41):7271-9).

In other embodiments, the sF gene sequence (e.g. in a plasmid) can be designed with optimized mammalian codons to remove cryptic splice sites and cryptic polyadenylation sites. Optimization also enhances translation by choosing codons that are used most frequently in the host cell being used. This type of “optimized” gene sequence can be expressed in the nucleus of the host cell. We have also optimized the F gene from the RSV Long strain, enabling us to produce the Long strain F protein from a plasmid in the nucleus. Many other examples of optimized genes can be found in the literature, including the first description of the human immunodeficiency virus gp160 gene (Haas et al. 1996 Curr. Bio 6:315-24). Such optimized genes can also be obtained commercially, where a company can synthesize genes for a fee, optimizing them as described to avoid cryptic splice sites and cryptic polyadenylation sites.

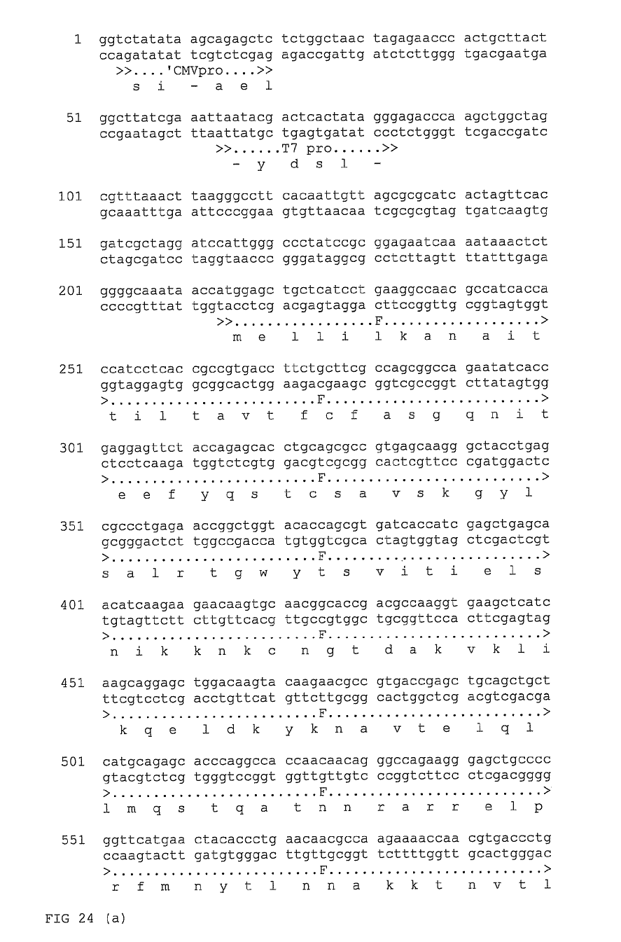

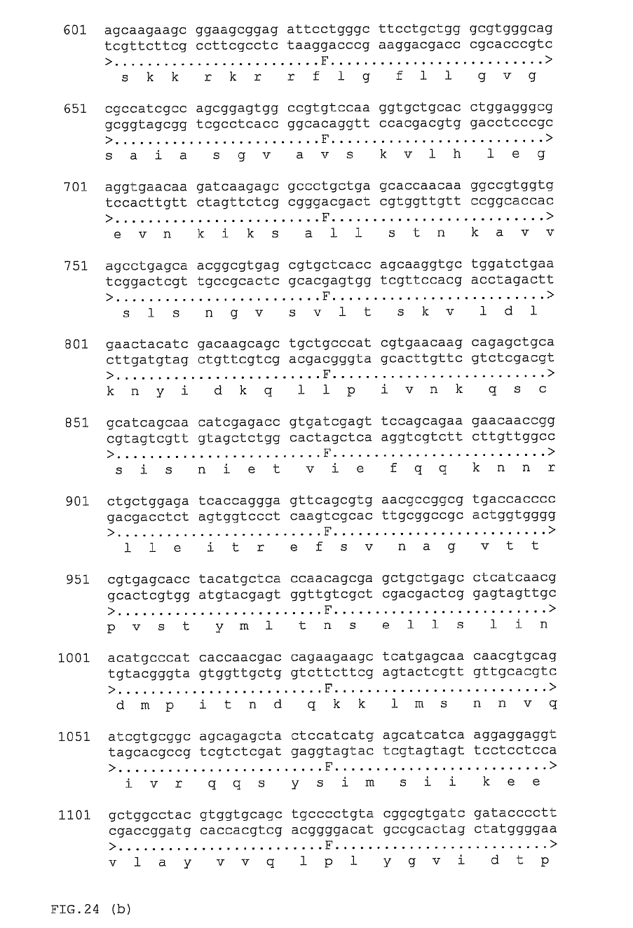

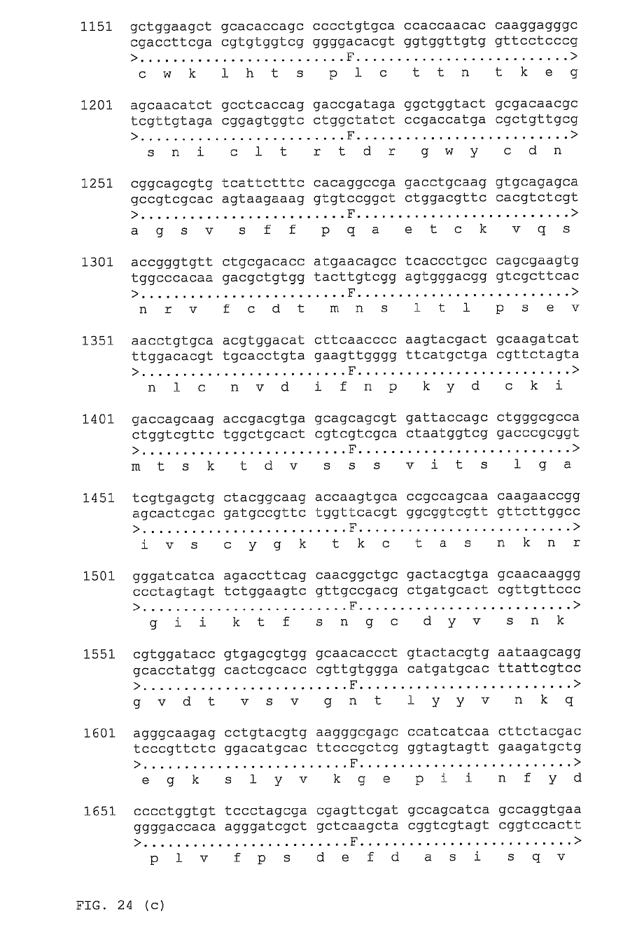

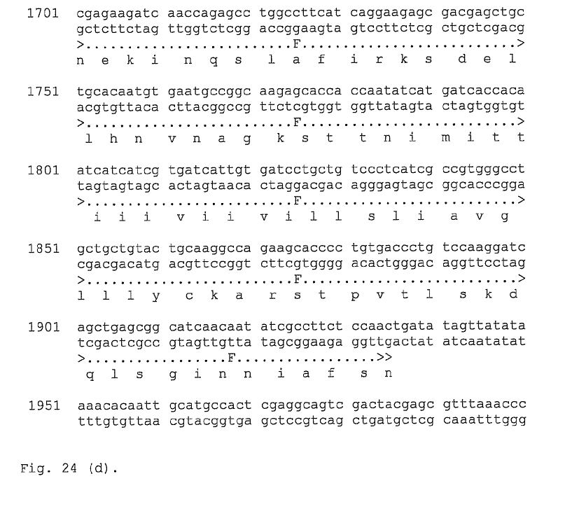

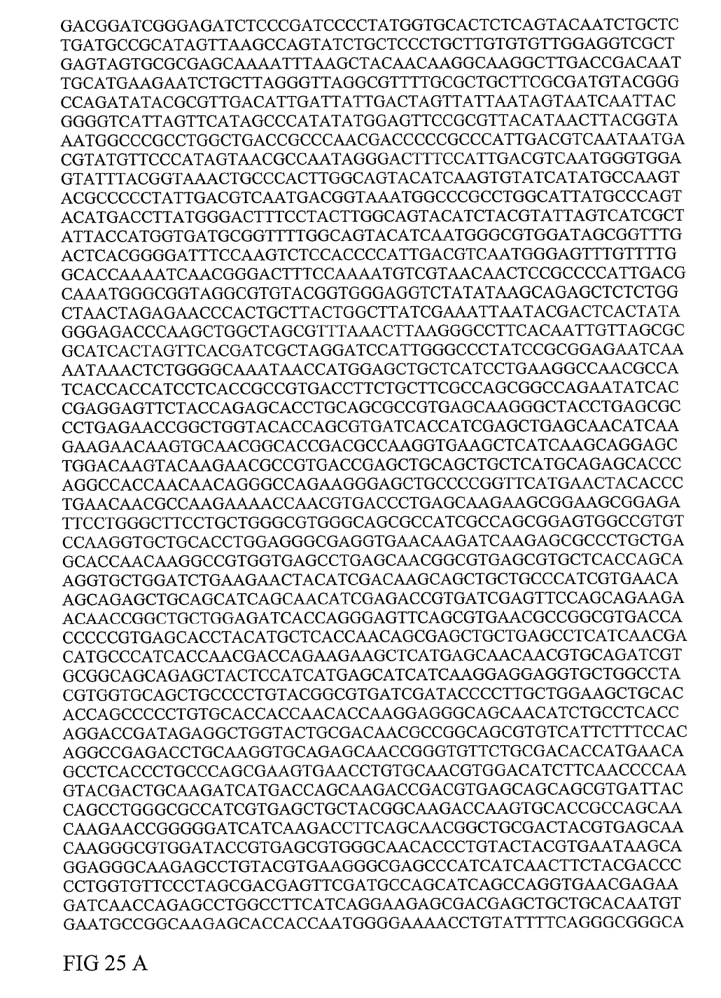

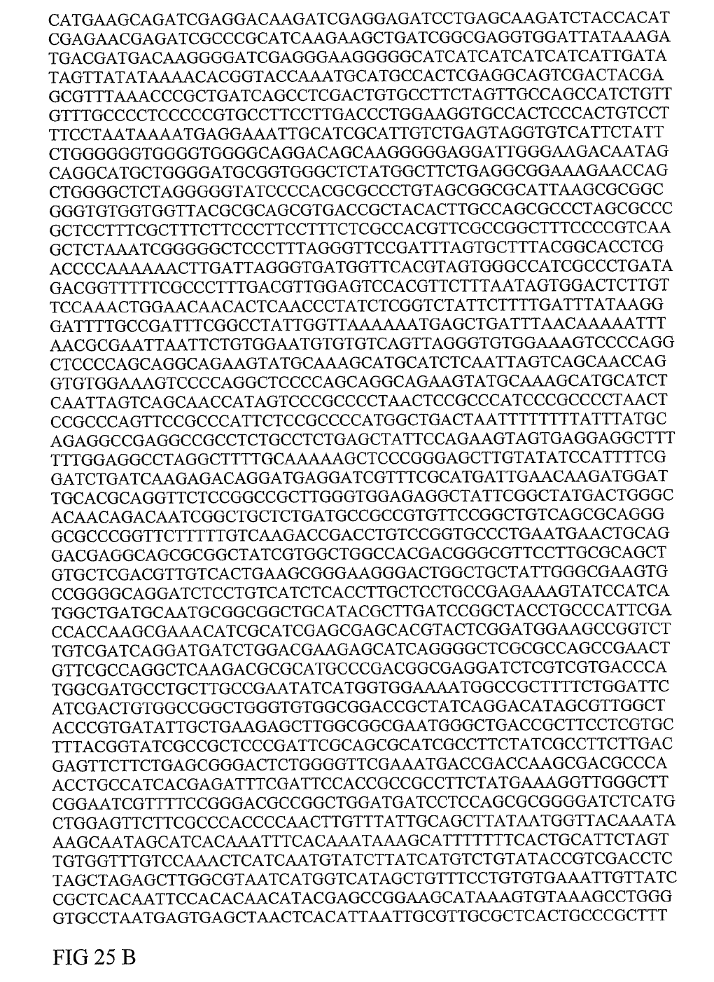

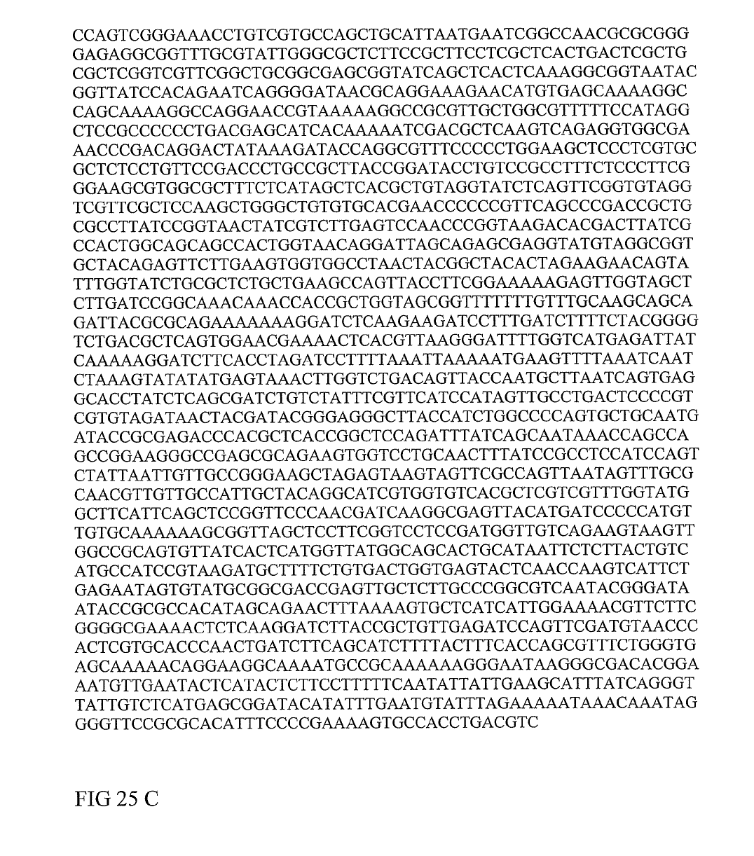

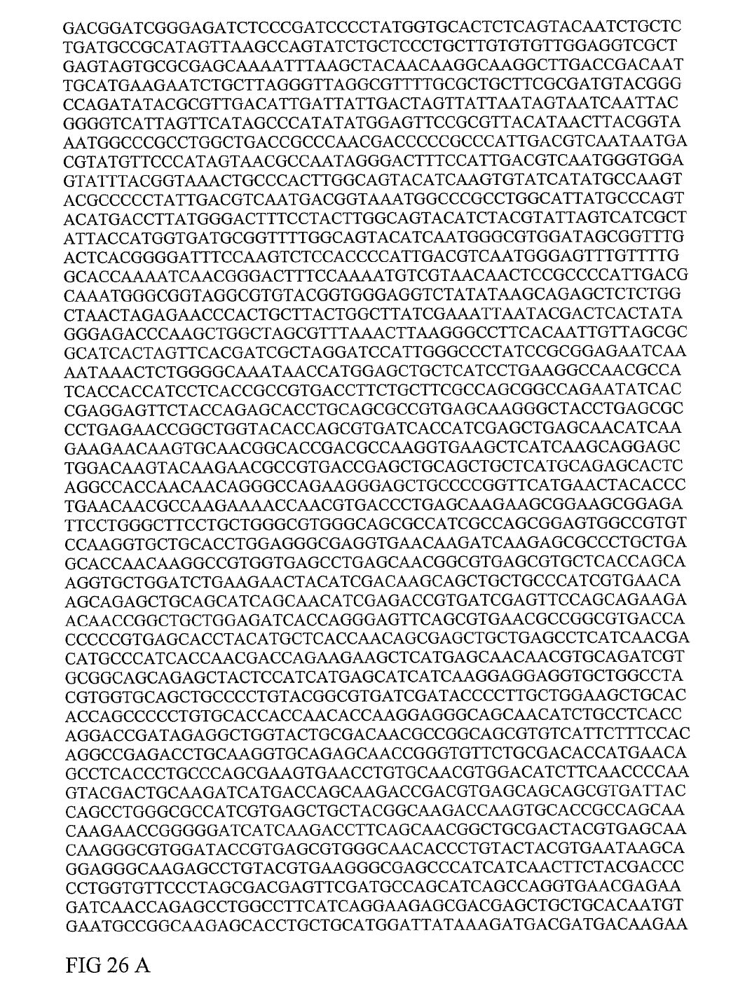

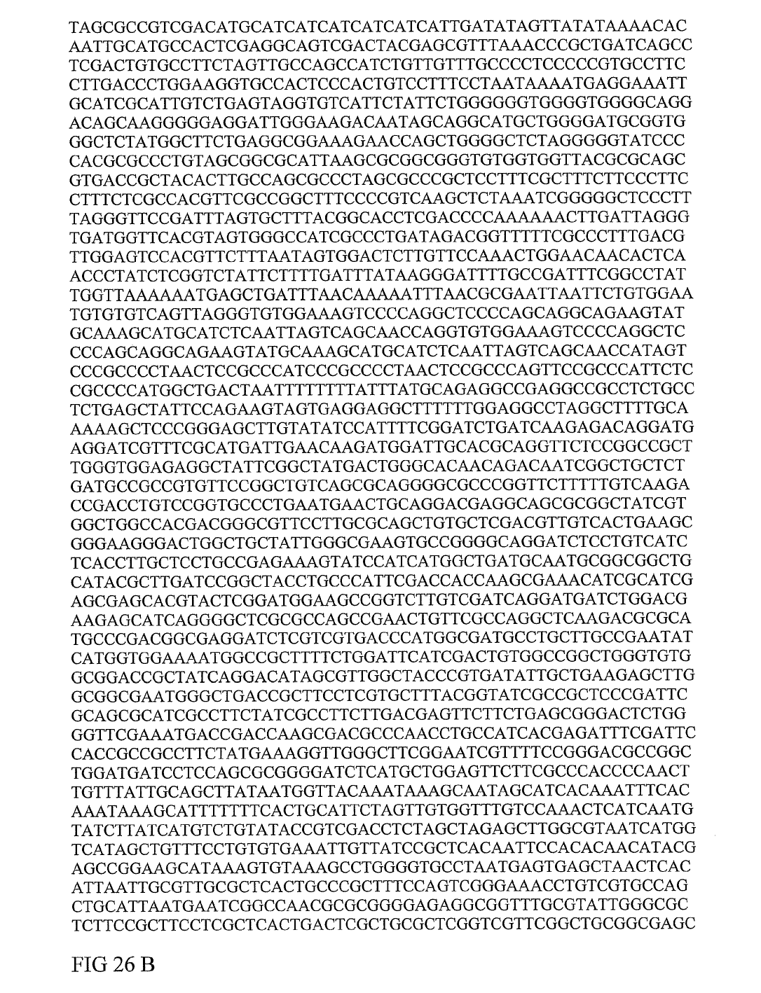

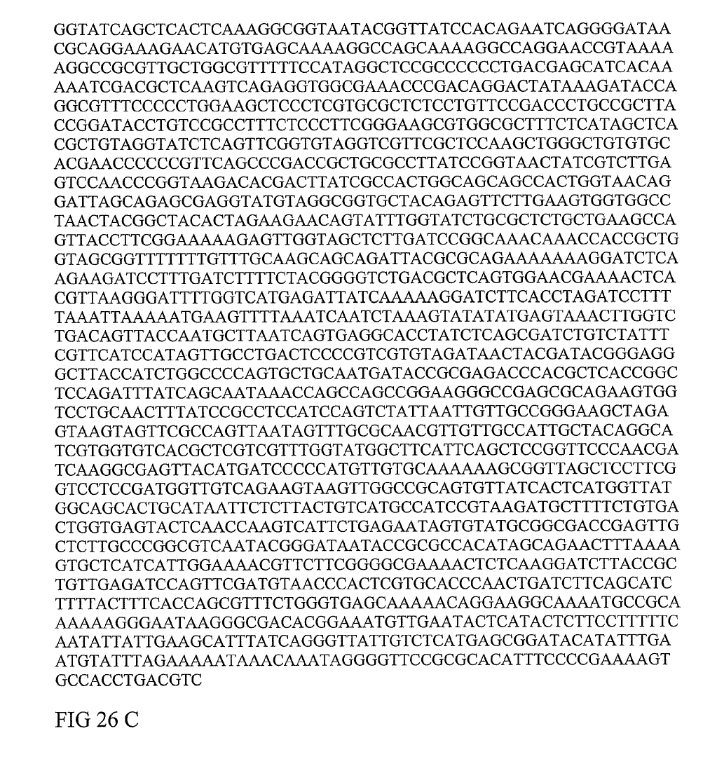

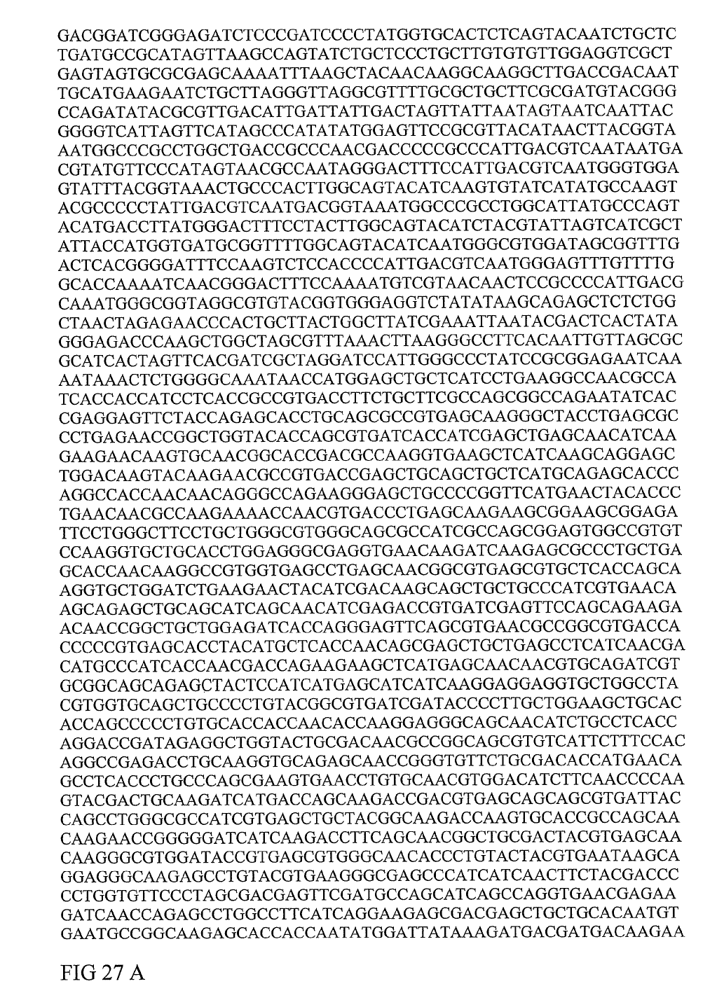

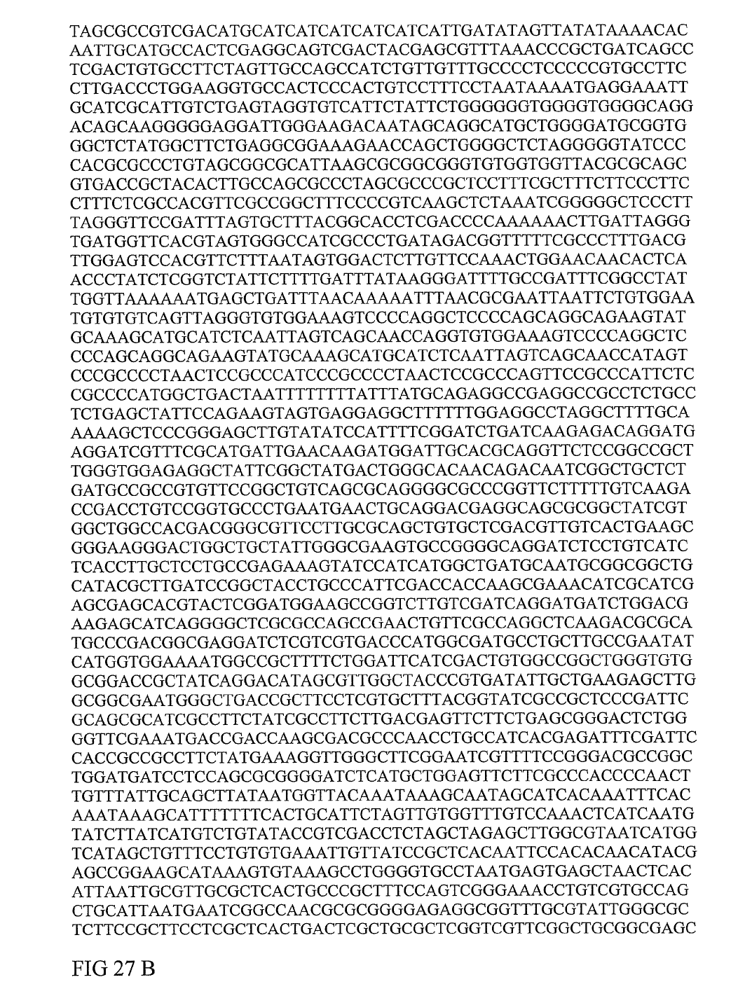

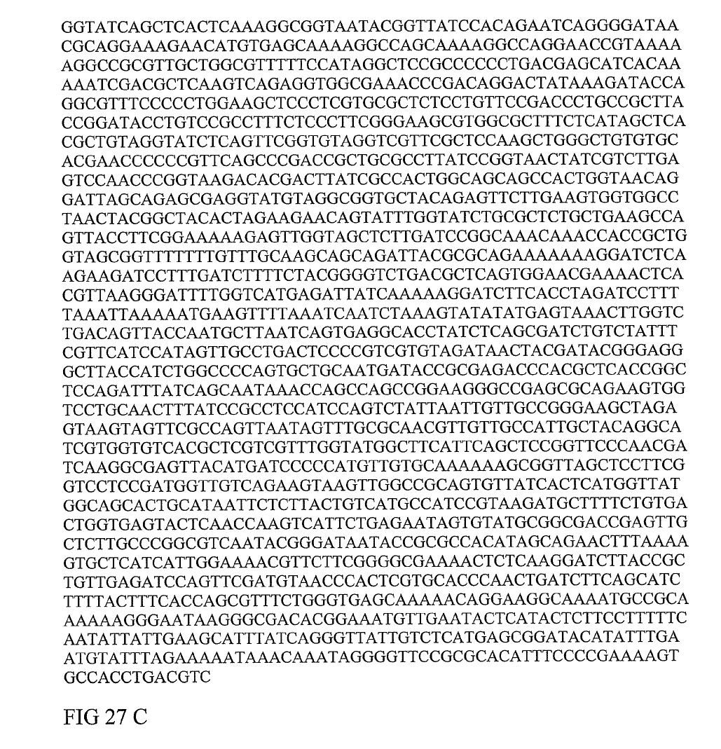

In one embodiment, the optimized F gene sequence is at least 85%, 90%, 91%, 92%, 93%, 94%, 95%, 96%, 97%, 98%, 99% or 100% identical to the sequence in FIG. 24 (SEQ ID NO: 6).

Using the computational structure of the F protein to design and/or screen potential anti-viral agents

Contemplated herein are methods of identifying a potential paramyxovirus antiviral agent that can bind a CRAC domain of a viral fusion (F) protein, including the step of using a three-dimensional structural representation as defined by the coordinates in Table 4 of a any one of the soluble or full-length pre- or post-triggered RSV F-protein, or a fragment thereof, which contains a cholesterol-binding CRAC pocket to computationally screen candidate compounds for an ability to bind the CRAC pocket.

This disclosure also contemplates a method of selecting a potential paramyxovirus antiviral agent, comprising the steps of providing a computer-generated model of the three-dimensional structure of any one of the soluble or full-length pre- or post-triggered RSV F-protein as defined by the atomic coordinates of RSV F-protein according to Table 4 and selecting chemical structures capable of associating with a CRAC domain having the sequence V/L/I-X₁₋₅-Y/F/W-X₁₋₅-R/K (SEQ ID NO: 40) in any one of the soluble or full-length pre- or post-triggered RSV F-protein computer-generated models.

Also contemplated herein is a method for selecting a paramyxovirus antiviral agent comprising generating a three-dimensional model of any one of the soluble or full-length pre- or post-triggered RSV F-protein as defined by the atomic coordinates of RSV F-protein according to Table 4 based at least in part on a predetermined sequence, selecting a CRAC domain defined by the atomic coordinates of RSV F-protein according to Table 4 for receiving the agent, and selecting at least one chemical structure compatible with the CRAC domain to define the agent. In some embodiments, the predetermined sequence is V/L/I-X₁₋₅-Y/F/W-X₁₋₅-R/K (SEQ ID NO: 40).

Also contemplated herein is a method comprising selecting a CRAC domain in a three-dimensional model of any one of the soluble or full-length pre- or post-triggered RSV F-protein as defined by the atomic coordinates of RSV F-protein according to Table 4 for receiving a paramyxovirus antiviral agent, and selecting at least one chemical structure compatible with the CRAC domain to define the agent. In some embodiments, the three-dimensional model of the protein is based at least in part on a predetermined sequence. In some embodiments, the predetermined sequence is V/L/I-X₁₋₅-Y/F/W-X₁₋₅-R/K (SEQ ID NO: 40).

Another embodiment contemplated herein is a method for assembling a potential paramyxovirus antiviral agent, comprising the steps of providing a computer-generated model of the three-dimensional structure of any one of the soluble or full-length pre- or post-triggered RSV F-protein as defined by the atomic coordinates of RSV F-protein according to Table 4, identifying a portion of at least one chemical structure, wherein the portion is capable of associating with a CRAC domain of any one of the soluble or full-length pre- or post-triggered RSV F-protein having the sequence V/L/I-X₁₋₅-Y/F/W-X₁₋₅-R/K (SEQ ID NO: 40), and assembling the identified portions into a single molecule to provide the chemical structure of the potential paramyxovirus antiviral agent.

Another embodiment contemplated herein is a method for assembling a paramyxovirus antiviral agent comprising generating a three-dimensional model of any one of the soluble or full-length pre- or post-triggered RSV F-protein as defined by the atomic coordinates of RSV F-protein according to Table 4 based at least in part on a predetermined sequence, selecting a CRAC domain defined by the atomic coordinates in Table 4 for receiving the agent and identifying at least a portion of at least one chemical structure compatible with the CRAC domain and assembling portions of chemical structures identified above into a molecule defining a chemical structure for the agent.

Also contemplated herein is a method for selecting a paramyxovirus antiviral agent comprising processing three-dimensional coordinates of a CRAC domain of a three-dimensional model of any one of the soluble or full-length pre- or post-triggered RSV F-protein to generate a criteria data set, comparing the criteria data set to one or more chemical structures of potential agents, and selecting the chemical structure from the comparing above that binds to the criteria data set to define the agent.

Another embodiment contemplated herein is a method for selecting a paramyxovirus antiviral agent comprising processing three-dimensional coordinates of a CRAC domain of a three-dimensional model of any one of the soluble or full-length pre- or post-triggered RSV F-protein to generate a criteria data set, comparing the criteria data set to at least one portion of one or more chemical structures of potential agents; and selecting at least one or more portions of chemical structures from the comparing above that bind to the criteria data set to define the agent.

Also contemplated herein are methods of identifying a compound that can bind a CRAC domain of a viral fusion (F) protein, comprising the step of using a three-dimensional structural representation of a pre-triggered soluble F protein, or a fragment thereof, which contains a cholesterol binding CRAC pocket to computationally design a synthesizable candidate compound that binds the CRAC pocket.

The computational design can include the steps of: identifying chemical entities or fragments capable of associating with the CRAC binding site; and assembling the chemical entities or fragments into a single molecule to provide the structure of the candidate compound. Also contemplated are methods of synthesizing any such candidate compound, and screening the candidate compound for F protein binding activity. Examples of such compounds include cholesterol derivatives or mimics. Cholesterol mimics include molecules that have similar contact points as cholesterol, but may be very different structurally.

Another example of such compounds includes compounds that are capable of displacing a preloaded cholesterol molecule in a CRAC pocket, causing the F protein to trigger prematurely.

In one example, the CRAC domain may comprise three CRAC1 motifs located in a pit at the top of the F protein trimer crown. Each CRAC1 motif has the sequence V/L/I-X₁₋₅-Y/F/W-X₁₋₅-R/K (SEQ ID NO: 40), or V/L/I-X₁₋₅-Y/F/W-X₁₋₅-D/E (SEQ ID NO: 41).

In one example, the CRAC containing virus is a paramyxovirus. In another example, the virus belongs to the pneumovirus subfamily virus. In yet another example, the virus is human RSV.

The three-dimensional structure model of a CRAC containing protein and a potential ligand may be examined through the use of computer modeling using a docking program such as FLEX X, DOCK, or AUTODOCK (see, Dunbrack et al., Folding & Design, 2:R27-42 (1997); incorporated by reference herein), to identify potential ligands and/or inhibitors. This procedure can include computer fitting of potential ligands to the ligand binding site to ascertain how well the shape and the chemical structure of the potential ligand will complement the binding site. [Bugg et al., Scientific American, December:92-98 (1993); West et al., TIBS, 16:67-74 (1995); incorporated by reference herein]. Computer programs can also be employed to estimate the attraction, repulsion, and steric hindrance of the two binding partners (i.e., the ligand-binding site and the potential ligand). Generally the tighter the fit, the lower the steric hindrances, and the greater the attractive forces, the more potent the potential drug since these properties are consistent with a tighter binding constant. Furthermore, the more specificity in the design of a potential drug, the more likely that the drug will not interact as well with other proteins. This will minimize potential side-effects due to unwanted interactions with other proteins.

A variety of methods are available to one skilled in the art for evaluating and virtually screening molecules or chemical fragments appropriate for associating with a protein. Such association may be in a variety of forms including, for example, steric interactions, van der Waals interactions, electrostatic interactions, solvation interactions, charge interactions, covalent bonding interactions, non-covalent bonding interactions (e.g., hydrogen-bonding interactions), entropically or enthalpically favorable interactions, and the like.

Numerous computer programs are available and suitable for rational drug design and the processes of computer modeling, model building, and computationally identifying, selecting and evaluating potential inhibitors in the methods described herein. These include, for example, GRID (available form Oxford University, UK), MCSS (available from Molecular Simulations Inc., Burlington, Mass.), AUTODOCK (available from Oxford Molecular Group), FLEX X (available from Tripos, St. Louis. Mo.), DOCK (available from University of California, San Francisco), CAVEAT (available from University of California, Berkeley), HOOK (available from Molecular Simulations Inc., Burlington, Mass.), and 3D database systems such as MACCS-3D (available from MDL Information Systems, San Leandro, Calif.), UNITY (available from Tripos, St. Louis. Mo.), and CATALYST (available from Molecular Simulations Inc., Burlington, Mass.). Potential inhibitors may also be computationally designed “de novo” using such software packages as LUDI (available from Biosym Technologies, San Diego, Calif.), LEGEND (available from Molecular Simulations Inc., Burlington, Mass.), and LEAPFROG (Tripos Associates, St. Louis, Mo.). Compound deformation energy and electrostatic repulsion, may be evaluated using programs such as GAUSSIAN 92, AMBER, QUANTA/CHARMM, AND INSIGHT II/DISCOVER. These computer evaluation and modeling techniques may be performed on any suitable hardware including for example, workstations available from Silicon Graphics, Sun Microsystems, and the like. These techniques, methods, hardware and software packages are representative and are not intended to be comprehensive listing. Other modeling techniques known in the art may also be employed in accordance with embodiments disclosed herein. See for example, N.C. Cohen, Molecular Modeling in Drug Design, Academic Press (1996) (and references therein), and software identified at internet sites including the CAOS/CAMM Center Cheminformatics Suite at www.caos.kun.nl, and the NIH Molecular Modeling Home Page at www.fi.muni.cz/usr/mejzlik/mirrors/molbio.info.nih.gov/modeling/software list/.

A potential ligand may be obtained from commercial sources or synthesized from readily available starting materials using standard synthetic techniques and methodologies known to those of ordinary skill in the art. The potential ligand may then be assayed to determine its ability to inhibit the target protein as described above. Synthetic chemistry transformations and protecting group methodologies (protection and deprotection) useful in synthesizing ligand compounds are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995); incorporated by reference herein.

The ligands described herein may contain one or more asymmetric centers and thus occur as racemates and racemic mixtures, single enantiomers, individual diastereomers and diastereomeric mixtures. All such isomeric forms of these compounds are expressly included in the present disclosure. The ligands described herein may also be represented in multiple tautomeric forms, all of which are included herein. The ligands may also occur in cis- or trans- or E- or Z-double bond isomeric forms. All such isomeric forms of such ligands are expressly included in the present disclosure. All crystal forms of the ligands described herein are expressly included in the present disclosure.

Whether a CRAC domain is empty or loaded with cholesterol, a compound that would “cap” or stabilize the trimer, blocking access to the cholesterol binding site, can prevent triggering. This stabilization can be temporary, or even permanent if the affinity is high enough. For example, if the F protein is pre-loaded with cholesterol, such a compound could bind to the three cholesterol hydroxyl groups that would be exposed at the top of the F protein trimer.

Therefore, contemplated herein are methods of identifying a compound that can stabilize the crown of a fusion protein. Such methods include the step of using a three-dimensional structural representation of a pre-triggered soluble F protein, or a fragment thereof, which contains a CRAC domain to computationally screen a candidate compound that is capable of stabilizing the crown of a fusion protein.

Also contemplated herein are methods of identifying a compound that can stabilize the crown of a fusion protein, comprising the step of using a three-dimensional structural representation of a pre-triggered soluble F protein, or a fragment thereof, which contains a CRAC domain to computationally design a synthesizable candidate compound that binds and is capable of stabilizing the crown of a fusion protein.

The computational design can include the steps of: identifying chemical entities or fragments capable of associating with the CRAC1 binding site; and assembling the chemical entities or fragments into a single molecule to provide the structure of the candidate compound.

In one example, the CRAC domain may comprise three CRAC1 motifs located in a pit at the top of the F protein trimer crown. Each CRAC1 motif has the sequence V/L/I-X₁₋₅-Y/F/W-X₁₋₅-R/K (SEQ ID NO: 40), or V/L/I-X₁₋₅-Y/F/W-X₁₋₅-D/E (SEQ ID NO: 41).

Compounds that stabilize the F protein, preventing triggering can be detected by their ability to inhibit changes in the structural indicator of sF proteins, or functional fragments thereof (e.g. circular dichroism or spectrofluorimetric spectrum) as discussed below.

Screening for candidate antiviral agents using soluble, pre-triggered F protein and fragments thereof.

Without wishing to be bound by theory, it is believed that the triggering mechanism works in one of two ways. If CRAC1 is empty, a compound that binds to CRAC1 will cause the F protein to either (i) trigger prematurely, leaving it spent and inactive and destroying the infectivity of the virion in whose membrane the F protein sits, or (ii) not trigger at all when it contacts a target cell membrane. If, on the other hand, the CRAC1 is pre-loaded with cholesterol, a compound that binds to CRAC1 more strongly than cholesterol, and so is capable of displacing cholesterol, would also reduce the infectivity of the virion by causing either (i) or (ii) above. In either case, such a compound can inhibit the biological activity of the fusion protein and reduce the infectivity of the virus.

Accordingly, contemplated herein are methods of screening for a candidate paramyxovirus antiviral agent using a soluble, pre-triggered F protein of a paramyxovirus, or fragments thereof, that comprise a CRAC1 domain having the sequence V/L/I-X₁₋₅-Y/F/W-X₁₋₅-R/K (SEQ ID NO:40) or V/L/I-X₁₋₅-Y/F/W-X₁₋₅-D/E (SEQ ID NO: 41). The method includes the steps of: (i) contacting a test agent with the soluble pre-triggered F protein or a functional fragment thereof; (ii) detecting a structural indicator of the soluble pre-triggered protein, or the fragment thereof, wherein a change in the structural indicator in the presence of the test agent as compared to the absence of the test agent indicates that the agent is a candidate antiviral agent for the paramyxovirus. In this method, the test agent would prematurely trigger the F protein, thereby reducing infectivity of the virus.

Alternative methods include screening for compounds that prevent RSV F protein triggering. The sF protein will likely be triggered by the addition of stimuli such as by incubation with lipid membranes, including liposomes, or by the addition of heat. Compounds that stabilize the sF protein can be detected by their ability to inhibit sF triggering when the sF protein is exposed to a triggering event. A structural indicator, as described above, can be used to detect conformational change in the F protein. The positive control for these screening assays can be sF protein heated or exposed to liposomes in the absence of any test compound. These assays could easily be adapted for high throughput to identify compounds that stabilize the sF protein, as described above for compounds that trigger the sF protein.

Thus, in another embodiment, the method of screening includes: (i) contacting a test agent with the soluble pre-triggered F protein of a paramyxovirus, or a fragment thereof, to form a test sF protein; (ii) exposing the test sF protein to a triggering event; and (iii) assessing a structural indicator of the test sF protein before and after exposure to the triggering event, wherein an absence of change in the structural indicator of the test sF protein after exposure to the triggering event indicates that the agent is a candidate antiviral agent for RSV. The absence of change in any individual sF protein indicates that the sF protein did not trigger, i.e. is incapable of triggering after contact with the test compound. Therefore, this screening method would identify compounds that can block the activity of the F protein, thereby reducing or blocking the infectivity of the virus.

The control in this method would be an sF protein that has not been contacted with the test agent but has been contacted with a control substance similar to but lacking the test agent. In this case, the sF protein would exhibit a change in the structural indicator after the triggering event.

A candidate antiviral agent is a compound that is capable of reducing the infectivity of the paramyxovirus when administered to a subject infected, or at risk of being infected, with the paramyxovirus. In some embodiments, the antiviral agent is an anti-RSV agent.

As used herein, “triggering” refers to the conformational change when an isolated soluble F protein, or functional fragment thereof, goes from a pre-triggered conformation to a post-triggered conformation, as shown in FIGS. 2 and 3. Thus, a soluble F protein or its functional fragment can undergo a conformational change even if they lack various portions of the F protein, including the fusion peptide.

In some embodiments, the steps of either of the methods described above are performed in the absence of an attachment protein.

The “structural indicator” as used herein refers to a parameter that is capable of detection and that indicates whether the F protein, or functional fragment thereof, has or has not undergone a conformational change as a result of being triggered. Detecting a difference between the structural indicator of an F protein, or functional fragment thereof, before as compared to after exposure to a test agent is indicative of a conformational change in the F protein (i.e. indicates that the test agent has triggered the F protein). Alternatively, the absence of change in the structural indicator after the F protein, or functional fragment thereof, has been exposed to both the test agent and a triggering event indicates that the F protein is not capable of changing its conformation, (i.e., the test agent has locked the F protein in its pre-triggered form.

The methods use a pre-triggered, soluble F (sF) protein or a functional fragment thereof, as described above. Described below, and in Table 2, is a non-limiting list of examples of screening methods, as well as examples of fragments that can be used in such screening methods.

Any of the following assays could easily be adapted to a 96-well or 384-well or similar format for high throughput screening. In this way, many compounds can be simultaneously and quickly assayed for their abilities to trigger or block the sF protein. A library of compounds related to cholesterol or cholesterol mimics, or any other library of chemical compounds can be rapidly tested in this way to identify lead compounds.

TABLE 2
sF protein triggering/blocking assays

| | | | Minimal |
|---|---|---|---|
| Assay | Required Domains | Domains not required | Sequence |
| Circular | F2: the two cysteines (C37 and C69) that | The signal peptide | 37-69 and |
| dichroism | link F2 to F1 and the sequence between | (SP): M1-G25 | 156-439 |
| (CD) | them. | F2: Q26-T36 and N70- | |
| | F1: The sequence from K156 to C212 that | R109 | |
| | includes the CRAC1 domain, the α-helix | pep27: E110-R136 | |
| | that includes C212 and the non-helical | F1: The fusion peptide | |
| | peptides N terminal and C terminal to the | (FP) F137-S155 | |
| | CRAC1 domain that become α-helical | F1: D440 through the C | |
| | upon triggering. | terminus, includes | |
| | F1: the two cysteines (C212 and C439) | HR2, the | |
| | that link F1 to F2 and the sequence | transmembrane domain | |
| | between them. | (TM) and the | |
| | The cysteines C37, C69, C212 and C439 | cytoplasmic tail (CT) | |
| | can be replaced by amino acids that | | |
| | interact in a non-covalent manner to hold | | |
| | F2 and F1 fragments together. | | |
| Spectro- | Same as above | Same as above | 37-69 and 156-439 |
| fluorimetry | | | (Trp1 is W52; Trp2 |
| | | | is W314; Trp3 is |
| | | | W341 is) Y198 in |
| | | | CRAC can be |
| | | | modified to Trp |
| Resonance | Same as above | Same as above | Same as |
| Raman (RR) | | | above |
| Spectroscopy | | | |
| Split GFP | Same as above | Same as above | 37-69 and |
| | | | 156-439 |
| FRET | Same as above | Same as above | 37-69 and |
| | | | 156-439 |
| Enzyme | Same as above | Same as above | 37-69 and |
| immunoassay | | | 156-439 |
| (EIA) | | | |
| Hydrophobic | Same plus in F1: The FP (F137-S155) or a | Same except for the FP | 37-69 and |
| die binding | similar fusion peptide from another virus | | 137-439 |
| | or a similar sequence | | |
| Liposome | Same plus in F1: The FP (F137-S155) or a | Same except for the FP | 37-69 and |
| association | similar fusion peptide from another virus | | 137-439 |
| | or a similar sequence | | |
| Hydrophobic | Same plus in F1: The FP (F137-S155) or a | Same except for the FP | 37-69 and |
| association | similar fusion peptide from another virus | | 137-439 |
| | or a similar sequence | | |

Functional assays

| | | | |
|---|---|---|---|
| Cell-cell | SP | SP can be replaced | 1-574 |
| fusion | F2 proteinF1 protein | with another SP | |
| | | The FP (F137-S155) | |
| | | can be replaced by a | |
| | | fusion peptide from | |
| | | another virus or a | |
| | | peptide with similar | |
| | | characteristics | |
| | | pep27 can be removed | |
| | | The CT can be replaced | |
| | | with the CT of another | |
| | | transmembrane protein | |
| | | that has its C terminus | |
| | | in the cytoplasm, but a | |
| | | CT from some protein | |
| | | is necessary | |
| Virus | Same as above | Same as above except | 1-574 |
| infection | | that a portion of the | |
| | | cytoplasmic domain is | |
| | | required for virus | |
| | | assembly, probably | |
| | | interaction with the M | |
| | | protein | |

The screening methods described above can use one or more structural indicators as follows:

Circular dichroism (CD). In one example, the structural indicator is circular dichroism (CD) spectrum of the protein.

In general, triggering converts the three short helices with their intervening non-helical regions into the long HR1 α-helix. The CD spectrum of a protein is highly sensitive to the secondary structure of the protein backbone. α-helical structure, β-sheet structure, and random coil have distinct, signature spectra. The conformational change upon triggering of the F protein converts several unstructured regions, and 2 β-sheets into a continuous α-helix. This increase in α-helicity and corresponding decrease in other structural components can be detected by change in the CD spectrum.

Fluorescence Emission. In another example, the structural indicator is the fluorescence emission of the sF protein as determined by, for example, spectro fluonimetory. Tryptophan (Trp) residues are responsible for the majority of a protein's fluorescent emission spectrum. When a solvent (polar environment) exposed Trp is excited in the range of 280 nm, the wavelength of maximum Trp emission is approximately 350 nm. When the same Trp is exposed to a hydrophobic environment, instead, the maximum emission is blue shifted.

The F protein contains 3 Trp residues (for a total of 9 in the trimer). Trp1 (W52) and Trp2 (W 314) are situated on the inside of the head, in the vicinity predicted for the fusion peptide, post-cleavage. Trp 3 (W 341) is situated on the exterior face of the F protein, pointing into the inter-domain interface that is also occupied by the N-terminus of the HR1 domain in the pre-triggered form.

If the structural changes of the sF protein alters the hydrophobicity of the environment of any of the three tryptophan residues, one or more spectral peaks will change their fluorescence value. Therefore, in another example, the structural indicator includes environmental monitoring of one or more tryptophan residues, Trp1, Trp2, or Trp 3, within the F protein. The environmental monitoring can include detecting a fluorescence emission shift effect and/or intensity change shown by one or more of the tryptophan residues.

In the case of sF protein, upon triggering, the hydrophobic fusion peptide is removed thereby changing the local environment of Trp1 and Trp2, exposing them to the solvent in the interior cavity of the F protein head. This polar environment will cause a shift in the emission spectrum that can be detected by a spectrofluorimeter as a measure of triggering. The local environment of Trp3 does not change significantly, as it remains on the solvent-exposed face of the protein in the post-triggered form. Therefore, Trp3 fluorescence could be used as a control.

In addition, we have found that tryptophan can replace the central tyrosine in the CRAC motif without loss of fusion activity. If the sF protein releases the bound cholesterol molecule when it is triggered, an sF molecule with tryptophan in this position will dramatically change its fluorescence.

In addition to the fluorescence emission shift effect shown by Trp residues in hydrophobic environments, Trp fluorescence is significantly quenched by contact with Asp and Glu residues. Trp 1 and Trp2 are near several Glu and Asp residues in the pre-triggered form, but are shielded from others by the interposing fusion peptide. When the fusion peptide is removed during triggering, Trp1 and Trp2 are exposed to these additional Asp and Glu contacts, resulting in significant quenching of the Trp1/Trp2 emission spectra.

Trp3 does not have any nearby Asp or Glu residues in either the pre or post-triggered F protein. In some embodiments, as an additional triggering monitor, an Asp or Glu residue could be engineered into HR1 at the point of contact with Trp3 in the pre-triggered form. Upon triggering, HR1 is dramatically removed from the neighborhood of Trp3, thereby removing the quenching effect of such an engineered quenching partner, and greatly increasing the intensity of the Trp3 emission spectrum.

Resonance Raman (RR) spectroscopy. Another example of structural indicator that involves environmental monitoring includes resonance Raman (RR) spectroscopy of the tryptophan residues.

Resonance Raman (RR) spectroscopy may be used for monitoring the microenvironment of specific amino acids. RR spectroscopy is based on scattering rather than emission. Generally, a monochromatic laser is used to excite the sample. Light from the laser interacts with vibrational, electronic or other transitions of the system, resulting in the energy of some photons being changed. The particular changes observed are indicative of the available excitation states in the sample. The excitation states of some amino acids (including Trp and Tyr) are sufficiently distinct that they may be excited, and therefore monitored, separate from each other and from the bulk of the protein. Because each residue's microenvironment affects its available excitation states, RR spectroscopy is another method that can used to selectively monitor the environment of Trp1/2/3 thereby detecting sF triggering.

Monitoring the environment of Trp 1/2/3 provides several assay mechanisms for observing the conformational change involved in triggering (extension of the HR1 helix). For example, the triggering initiation event, removal of the cholesterol from the CRAC domain, would effect a dramatic change in the local environment of the CRAC Tyr. Monitoring this Tyr therefore provides an assay mechanism for the triggering initiation event, rather than the triggering conformational change monitored by Trp1/2/3. Therefore, in yet another example, the structural indicator includes environmental monitoring of the CRAC region's central tyrosine residue. The environmental monitoring can be resonance Raman (RR) spectroscopy of the tyrosine residue. Alternatively, the tyrosine residue can be replaced by a tryptophan (Trp 4) and the environmental monitoring can be detecting a fluorescence emission shift effect shown by such Trp4 residue upon removal of cholesterol from the neighborhood of the CRAC domain.

Hydrophobic dye binding. Yet another example involves exposing the test F protein to a hydrophobic dye wherein the structural indicator is fluorescence of the hydrophobic dye. Examples of hydrophobic dyes include 8-anilinonaphthalene sulfonate (ANS), Sypro Orange, or a similar dye. Hydrophobic dyes are transparent in an aqueous environment, but display increasing fluorescence as the character of their environment becomes more hydrophobic. These dyes are commonly used to monitor the denaturation temperature of soluble proteins, as the loss of tertiary structure exposes hydrophobic regions of the proteins that would usually be buried and inaccessible to the dye. Upon binding to the hydrophobic regions, such a dye will fluoresce, signaling the change in structure. Hydrophobic dyes such as ANS or Sypro Orange can be used to monitor the onset of availability of these hydrophobic regions, thereby monitoring the conformational change caused by triggering. During the F protein triggering event the highly hydrophobic fusion peptide will become exposed, a hydrophobic dye will bind and fluoresce.

Liposome association. In another example, the structural indicator involves binding of the test F protein with a liposome membrane. Triggering of the sF protein exposes its fusion peptide. The highly hydrophobic fusion peptide will insert itself into the hydrophobic core of any available membrane. If liposomes are available, the fusion peptides will insert into these artificial membranes causing the sF protein to associate with the liposomes. The liposomes can be separated from the unbound sF protein by flotation centrifugation, by column chromatography, or other methods. The sF protein may also be triggered at some unknown rate by contact with lipid membranes, such as liposomes. For this reason, a test compound would most likely need to be added to the sF protein before exposing sF to the liposomes. Exposure of the pre-triggered F protein to liposomes can also cause some of the F molecules to trigger and could be used as an assay to identify compounds that block triggering.

Hydrophobic association. In another example, the structural indicator involves hydrophobic association. The surface of the pre-triggered sF protein is hydrophilic, like the surface of most proteins. However, when the sF protein is triggered, its fusion peptide is exposed. The fusion peptide is highly hydrophobic and hydrophobic surfaces have a strong attraction for other hydrophobic surfaces. Therefore, a structural indicator assay can use plates or beads with a hydrophobic surface, to which the post-triggered sF protein, but not the pre-triggered sF protein will bind. In one example of this assay an aliquot of pre-triggered sF protein in solution will be added to each well or bead. A test compound will be added and mixed. If the sF protein is triggered, it will expose its fusion peptide and bind to the hydrophobic surface of the well or bead. Unbound protein will be washed off and the bound protein can be detected. Various methods of detection are possible, including, but not limited to, detection by 6HIS (SEQ ID NO: 21) or FLAG M2 antibodies, or by antibodies that react specifically with the post-triggered sF protein. These antibodies can either be directly labeled with a detection molecule or detected by a secondary antibody labeled with a detection molecule. The detection molecule could be, for example but not limited to, a fluorescent molecule, such as fluorescene or rhodamine, or an enzyme. Binding of the fluorescent molecule can be detected by a fluorimeter. An enzyme, such as horseradish peroxidase or alkaline phosphatase can be detected by incubation with a corresponding substrate that is altered by the enzyme in a predictable manner, for example by turning color or by fluorescing, which can be detected in a spectrophotometer or fluorimeter, respectively. The sF protein could also be directly fused to a fluorescent moiety, such as a green fluorescent protein (GFP), or it can be chemically linked to a fluorescent molecule like fluoroscene or rhodamine, or fused to or chemically linked to an enzyme such as horseradish peroxidase or alkaline phosphatase.

Split GFP. In another embodiment, the structural indicator is the split GFP (Cabantous, S., et al. 2005. Nat Biotechnol 23:102-7) detection of the post-triggered sF protein. In the pre-triggered sF protein, the N and C termini of sF1 are far apart but they are brought together in the post-triggered form. In this method, one portion of GFP is fused to the sF protein fusion peptide sequence via a flexible linker, replacing both the furin cleavage site N terminal to the fusion peptide and pep27. Pep27 is the peptide between the two natural F protein furin cleavage sites that is normally removed during processing in the Golgi (FIG. 1). All or part of the fusion peptide may also be replaced. A furin cleavage site N terminal to the inserted GFP fragment replacing pep27 will remain intact and will be cleaved during passage through the Golgi. The second portion of GFP is fused to the C terminus of the sF molecule. In the pre-triggered sF protein, these two portions of GFP would be separated. Triggering followed by 6-helix bundle formation will bring the two GFP portions together, enabling GFP to fluoresce when struck with fluorescent light of the proper wavelength. This assay can function in solution without plate washing and without the addition of additional detection reagents.

FRET In another assay, the structural indicator comprises Forster Resonance Energy Transfer (FRET) (Piston, D. W., and G. J. Kremers. 2007. Trends Biochem Sci 32:407-14) detection of the post-triggered sF protein in which the N and C termini of the sF protein are brought together. The sF construct is similar to the construct described above for the split GFP approach, except that two complete or nearly complete fluorescent proteins are fused to the sF1 protein: one N terminal to the fusion peptide and replacing the pep27 sequence, the furin cleavage site and possibly the fusion peptide sequence; and the other at the C terminus of the sF protein (FIG. 1). A second furin cleavage site, N terminal to the first fluorescent protein, the same position as the furin site that previously preceded pep27, will remain intact and will be cleaved during passage through the Golgi. In the pre-triggered sF protein the two fluorescent proteins are separated, and because of the separation distance do not transfer energy. Following triggering, the two fluorescent proteins are brought together when the 6-helix bundle forms (FIG. 2). When this post-triggered sF protein molecule is struck with fluorescent light of the proper wavelength to cause one of the fluorescent molecules to fluoresce, its emission wavelength will excite the other fluorescent molecule and the emission wavelength of this molecule will be detected. Only when the two fluorescent molecules are in very close proximity will emission from the second fluorescent molecule be released and detected in a fluorimeter. This assay can function in solution without plate washing and without the addition of additional detection reagents. Several combinations of fluorescent proteins can function in this assay, including but not limited to cyan fluorescent protein and yellow fluorescent protein.

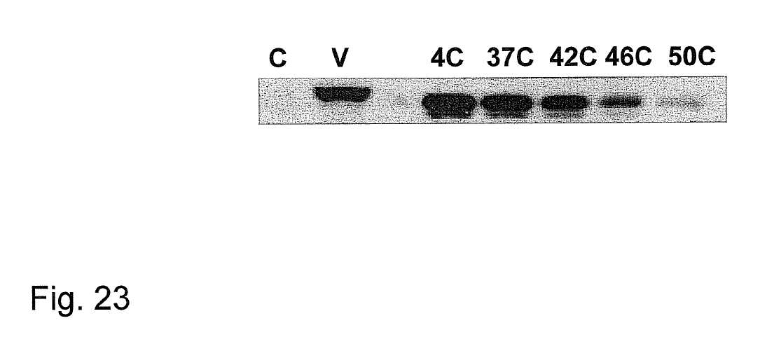

Enzyme immunoassay (EIA). In one example, the structural indicator is loss of antibody binding. Triggering the sF protein (for example by heat treatment) causes the sF protein to dramatically alter its conformation, as indicated by the loss of sF binding to neutralizing MAbs (FIG. 23). This loss of MAb binding can be used to detect a compound that causes sF protein triggering. For example, a 96-well assay plate is coated with the sF protein, or with a MAb to the FLAG tag or to the 6HIS tag (SEQ ID NO: 21) followed by incubation with the pre-triggered sF protein, washing between each addition. A test compound is added, and the remaining pre-triggered sF protein is detected with one of the neutralizing MAbs directly labeled with a fluorescent molecule, or with an enzyme followed by its substrate. If the compound does not cause triggering, the labeled neutralizing MAb will react with the sF protein. If the compound does cause triggering, this MAb will not react.

The ability of a compound to prevent sF protein triggering is tested in the same manner, i.e., following the addition of test compound, the plate is exposed to a triggering event (e.g. heat). Detection is performed in the same manner. If the neutralizing MAb binds to the sF protein, the test compound prevented triggering.

Alternatively, a 96-well assay plate is coated with a MAb to the post-triggered form of the sF protein. A solution of pre-triggered sF protein is added to the well along with a test compound. If the test compound causes the sF protein to trigger, the resulting post-triggered sF protein will bind to the MAb on the well. Unbound sF protein is washed off and the EIA is developed with a second MAb that also recognizes the post-triggered F protein but at a different antigenic site. The second MAb is directly labeled with a fluorescent molecule or an enzyme followed by its substrate.

The ability of a compound to prevent sF protein triggering is tested in the same manner, but following the addition of the sF protein solution and the test compound, the plate is exposed to a triggering event (e.g. heat). Detection is performed in the same manner. If the second, post-triggering specific, MAb detects the sF protein, the test compound did not prevent triggering. If this MAb does not detect the sF protein, the test compound prevented triggering.

Functional Assays

The primary assays, as described above, can be followed by functional assays that use the membrane-bound F protein to assess cell-cell fusion or viral infection of cells.

Cell-cell fusion. Expression of the complete F protein (with or without pep27) in cultured cells that are sensitive to viral infection causes the cells to fuse. The F protein can be expressed either by infecting with a virus or by transfecting transiently or stably with the F gene alone. Stable transfection with the F gene would likely require control with an inducible promoter to prevent fusion during cell growth and before addition of the test compounds. This assay can also be developed as a high throughput assay. The read-out can be by microscopic counting of syncytia.

This assay could include a gene for a protein whose presence is relatively simple to detect, such as luciferase, driven by a promoter which is normally switched off, in one cell line. A second set of cells containing the molecule needed to activate transcription of the detection gene can be added to the wells and incubated, usually for 4 to 12 hours to allow the F protein to cause fusion. At that point, the cells are lysed and the amount of enzyme generated is determined by the addition of substrate (see, for example, Nussbaum, O., et al. (1994). J Virol 68(9), 5411-22.). Such a cell-cell fusion assay could be used to screen for compounds that inhibit fusion.

Virus infection. Additional proof that a compound has antiviral activity is the demonstration that it inhibits infection of cultured cells. In another embodiment, compounds that are able to trigger the sF protein, or to prevent sF protein triggering, identified by the primary screening methods above, can be tested for their ability to prevent viral infection in a secondary screen. For example, in a high throughput assay, multi-well tissue culture plates such as 96-well or 386-well plates are seeded with cultured cells sensitive to paramyro virus infection and inoculated with a fixed number of infectious viruses, usually 30 to 100 plaque-forming units (pfu). Compounds are added before, with, or after virus addition. After a period of time, usually 1 to 3 days, the cells are fixed with a reagent such as methanol, stained with a dye such as methylene blue, and examined by microscope for small syncytia, the fused cells that result from infection. Alternatively, the cells can be stained with an antibody to one or more of the viral proteins. The antibody can be either directly labeled with a fluorochrome or with an enzyme whose substrate precipitates at the site, or can be detected by a secondary antibody that is linked to a fluorochrome or an enzyme.

Alternatively, a recombinant virus expressing a marker protein such as an enzyme, luciferase, β-galactosidase, or other, or a fluorescent protein, such as a green fluorescent protein, red fluorescent protein, or other can be used. In that case the number of infected cells can be counted with a microscope after an appropriate passage of time, e.g., the following day.

In an alternative embodiment, the inoculum can be a much higher amount of virus, usually averaging one or more pfu/cell. In this case, the plate can be analyzed the following day or later by a plate reader. Compounds that have no effect on virus infection result in bright fluorescence or large amounts of enzyme production detected by the addition of substrate, but compounds that inhibit viral replication will prevent the virus from expressing its fluorescent protein and the wells will be less bright or turn over less substrate. Detergent may be added to each well to enhance the accuracy of the reading by homogenizing the signal across each well.

When used as a secondary assay, these infectivity assays will be able to assess the antiviral activity of compounds identified in the sF protein triggering, and triggering inhibition, assays described above.

All of the screening methods described herein can be used for members of the paramyxovirus family whose F proteins contain CRAC domains, including, pneumoviruses or human RSV.

Also contemplated herein are compounds identified using the screening methods described above. Focused libraries of compounds representing the precursors to cholesterol or derivatives of cholesterol in their natural state or derivatized at any possible site or sites with formyl, acetyl, hydroxyl, or any other R group can be used to screen for active compounds. Likewise, focused libraries of compounds that make contacts with the CRAC domain that are similar to cholesterol and those that are derivatized at any possible site or sites with formyl, acetyl, hydroxyl, or any other R group can be used to screen for active compounds.

Compounds that inhibit the synthesis of cholesterol

Since cholesterol in a liposome membrane as a model of the target cell membrane enhances the ability of the F protein to trigger, blocking its synthesis in an infected cell would reduce or prevent infection of that cell. If cholesterol is incorporated into the F protein, blocking its synthesis in an infected cell would prevent incorporation into the F protein. For example, if incorporation into the F protein is necessary for the F protein to trigger when it contacts a target cell, the F protein that is produced in that cell and incorporated into virions would be unable to trigger and the virion would not be infectious. Alternatively, if incorporation of cholesterol stabilizes the F protein in its pre-triggered form, without cholesterol the F protein would be unstable and may trigger prematurely, preventing virion formation or allowing the formation of virions that are non-infectious.

Therefore any compound that can reduce or inhibit cholesterol synthesis can be a candidate antiviral compound capable of reducing or inhibiting the biological activity of the fusion protein. Accordingly, contemplated herein are compounds that can reduce, inhibit, or block cholesterol synthesis in infected cells, thereby reducing the biological activity or infectivity of the virus. Such a compound will have antiviral activity against a paramyxovirus that contains a CRAC domain. In one example, the paramyxovirus belongs to the pneumovirus subfamily. In another example, the paramyxovirus is human RSV. In another example, the paramyxovirus is PIV3, PIV1, or NDV.

In order that the embodiments disclosed herein may be more readily understood, the following examples are set forth. It should be understood that these examples are for illustrative purposes only and are not to be construed as limiting the embodiments disclosed in any manner.

### Example I

### Computer Modeling of the RSV F Protein

We used the following strategy to model the pre-triggered and post-triggered forms of the RSV sF protein based on the X-ray crystallographic structures of the PIV5 and PIV3 sF structures (Yin, et al. 2005. Proc Natl Acad Sci USA 102(26), 9288-93; Yin, et al. 2006 Nature 439(7072), 38-44 respectively (FIG. 2-6). We generated the pre-triggered model by threading the RSV F sequence onto the C chain of the PIV5 (SV5) PDB structure (PDB ID 2B9B), using a published F protein alignment (Day et al., 2006. Virol J. 3:34), with small modifications to correct a misalignment in the pep27 region, and internal to one disulfide loop. SwissModel (Schwede et al., 2003 Nucleic Acids Res. 31(13):3381-5) was used to independently generate structures for the F1 and F2 strands. These were combined using Quanta[MSI], and a mild energy minimization applied in Quanta to correct any large errors in bond angles and lengths. The trimer was generated using Pymol (Delano Scientific) by calculating RMS minimized transformations for the A and B chains, based on corresponding carbon-alpha atoms within the invariant region of the F protein head. The post-triggered model was generated in a similar fashion, using the C chain of the post-triggered human PIV3 PDB structure (PDB ID 1ZTM). As with Day's results (Day et al., 2006. Virol J. 3:34), our confidence in these models is enhanced by the fact that cysteines not present in the parent F proteins, are placed in appropriate proximity to form disulfide bonds in the models.

### Example 2

### Method for Generating Soluble RSV F Proteins

We generated three versions of the RSV sF protein from the full-length F protein gene of an RSV subgroup A strain virus. The virus gene was cloned as part of the complete RSV genomic cDNA clone, D46. The D46 F protein sequence is identical to the A2 strain of RSV (GenBank: X02221) with the exception of three amino acids. The F protein of A2 differs from D46 at E66K, P101Q, and F342Y, where the first letter represents the A2 amino acid, at the numbered position, and the final letter represents the D46 amino acid at that position.

We used a codon-optimized, synthetic version of the D46 RSV A2 F protein gene (from Peter Collins FIG. 24) to construct the gene expressing a soluble form of pre-triggered F protein (sF) in 3 versions. The F gene sequence in this plasmid was designed with optimal mammalian codons to enhance translation, and without cryptic splice or polyadenylation sites.

We removed the optimized F gene from the optiF plasmid by digestion with SacII and XhoI and inserted it into the same restriction sites in the expression plasmid, MP319, a modified version of pcDNA3.1+ (Invitrogen, Inc.) that we prepared by inserting these restriction sites into its multiple cloning site. A cytomegalovirus promoter preceded the F gene in this plasmid to drive its expression. The final cDNA clone, MP340, expressed the RSV F protein from the nucleus when transfected into mammalian HeLa or human embryonic kidney 293T cells.

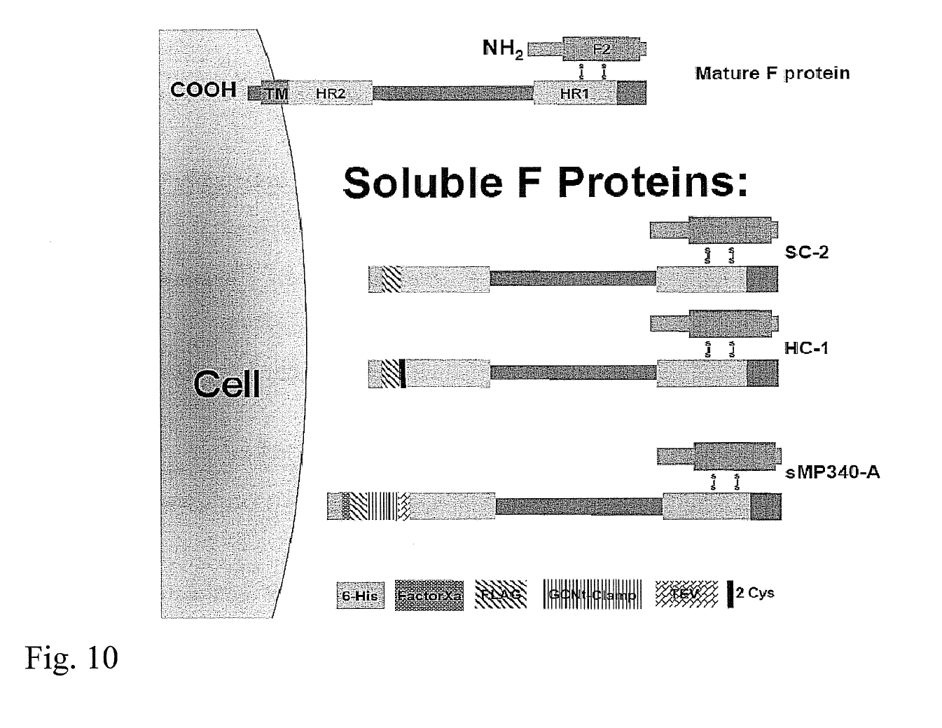

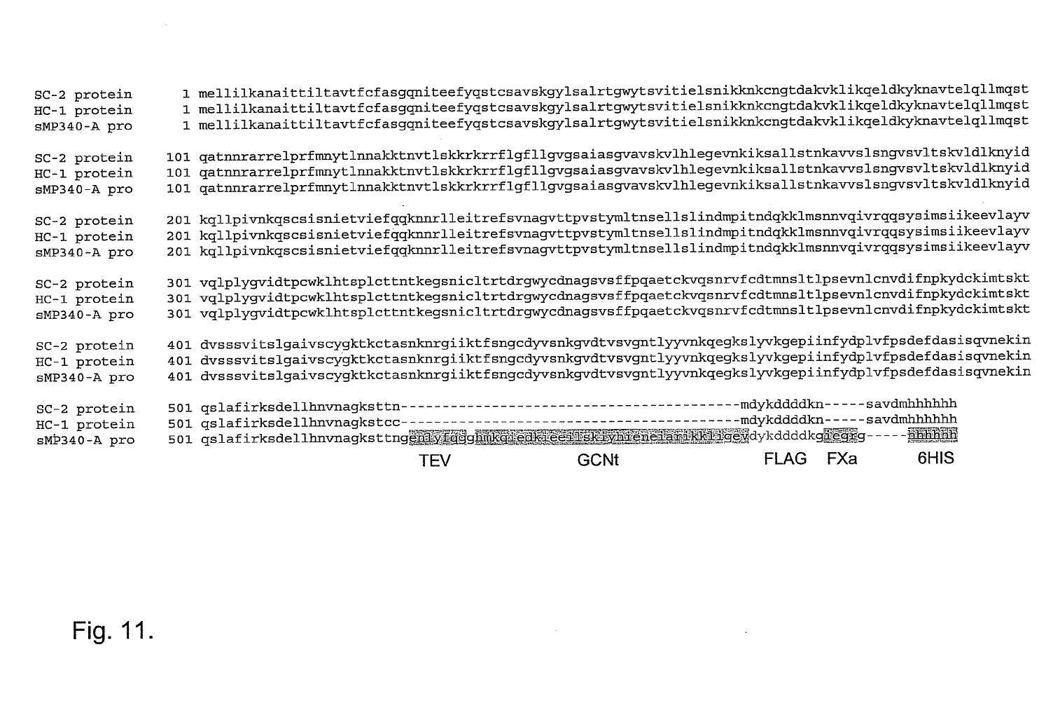

The three versions of the RSV sF protein (cartoon in FIG. 10; sequences in FIG. 11) were constructed from MP340 by replacing the transmembrane and cytoplasmic domain of the F protein gene: 1) with a FLAG tag followed by a 6-histidine (6HIS) tag (SEQ ID NO: 21) (SC-2); 2) and the last two amino acids of the HR2 helix (523 and 524) with two cysteine residues to allow the C terminus of the F sequence in the trimer to covalently link the monomers, followed by a FLAG tag followed by a 6HIS tag (SEQ ID NO: 21) (HC-1); and 3) with a TEV protease cleavage site followed by a GCNt trimerization domain followed by a FLAG tag followed by a Factor Xa cleavage site followed by a 6HIS tag (SEQ ID NO: 21) (sMP340-A). These novel sequences replacing the C terminus of the RSV F protein were designed to purify the sF protein released into the medium of transfected cells (6HIS tag (SEQ ID NO: 21) or FLAG tag), enable easy detection of the sF proteins (6HIS tag (SEQ ID NO: 21) or FLAG tag), or to clamp this end of the molecule to stabilize it (covalently with cysteines or non-covalently with the GCNt trimerization domain).

SC-1, the original sF gene that contains a FLAG tag followed by a 6HIS tag (SEQ ID NO: 21) at the C terminus of the F sequence, was constructed from the optimized F protein gene in pUC19 vector using inverse PCR mutagenesis (Byrappa, Gavin, and Gupta, 1995). The entire plasmid was amplified using two oligonucleotide primers (SEQ ID NO. 7 and 8) that include the sequence to be added (FLAG and 6HIS tags (SEQ ID NO: 21)), and set apart on the target plasmid to exclude the sequence to be deleted (transmembrane and cytoplasmic domains of the protein). The PCR product was purified, ligated, transformed into E. coli, and plated on bacterial medium-containing agar with ampicillin. Surviving clones were analyzed for plasmids containing the mutant sequence.

The first plasmid expressing sF protein, SC-2, was generated by digesting SC-1 with SacII and XhoI then inserting into the similarly digested MP340, pcDNA3.1 based expression vector (Invitrogen).

HC-1 was generated directly from SC-2 by inverse PCR mutagenesis with two oligonucleotide primers (SEQ ID NO. 9 and 10) to introduce two cysteine residues in place of the two C terminal amino acids of the F sequence in SC-2, in order to covalently link the three monomers within the trimer.

The sMP340-A construct was more complex to generate because it included a large stretch of novel sequence and there was no convenient restriction sites near the site of insertion of this new sequence. We assembled the new sequence as a series of 4 overlapping oligonucleotide sequences (SEQ ID NO. 11, 12, 13, and 14), amplified them through 7 cycles in a thermocycler by PCR, then took a small portion of this reaction and added two primers (SEQ ID NO. 15 and 16) from the extreme ends of the new segment and amplified the complete novel sequence. The primer (SEQ ID NO. 16) at the 3′ (right) end contains an XhoI site for insertion into the plasmid. Because there were no convenient restriction sites in the C terminus of the F gene, we PCR amplified a segment of the F gene that contained a ClaI site at its 5′ end and overlapped with the novel synthetic sequence at its 3′ end. We mixed this PCR product with the novel sequence and PCR amplified with primers (SEQ ID NO. 16 and 17) at the extreme ends of this final product. This final product was digested with ClaI and XhoI and inserted into the similarly digested MP340 to generate sMP340-A.

TABLE 3
Primers used in constructs

| | SEQ | |
|---|---|---|
| | ID | |
| Construct | NO | Sequence/comments |
| SC-2 | 7 | CTTGTCATCGTCATCTTTATAATCCATATTGGTGGTG |
| | | CTCTTGCCG |
| | | Reverse oligo containing FLAG tag |
| | | sequence |
| | 8 | AATAGCGCCGTCGACATGCATCATCATCATCATCATT |
| | | GATATAGTTATATAAAACACATTGC |
| | | Forward oligo containing 6HIS tag |
| | | (SEQ ID NO: 21) sequence |
| HC-1 | 9 | GGTGCTCTTGCCGGCATTCAC |
| | | Reverse oligo |
| | 10 | TGCTGCATGGATTATAAAGATGACGATGAC |
| | | Forward oligo |
| sMP340-A | 11 | CCAGCATCAGCCAGGTGAACGAGAAGATCAACCAGAG |
| | | CCTGGCCTTCATCAGGAAGAGCGACGAGCTGCTGCAC |
| | | AATGTGAATG |
| | | 1 of 4 overlapping oligos creating a |
| | | novel trimerization domain, two |
| | | protease recognition sites, FLAG and |
| | | 6HIS tags (SEQ ID NO: 21) |
| | 12 | CTCAGGATCTCCTCGATCTTGTCCTCGATCTGCTTCA |
| | | TGTGCCCGCCCTGAAAATACAGGTTTTCCCCATTGGT |
| | | GGTGCTCTTGCCGGCATTCACATTGTGCAGCAG |
| | | 1 of 4 overlapping oligos creating a |
| | | novel trimerization domain, two |
| | | protease recognition sites, FLAG and |
| | | 6HIS tags (SEQ ID NO: 21) |
| | 13 | CAAGATCGAGGAGATCCTGAGCAAGATCTACCACATC |
| | | GAGAACGAGATCGCCCGCATCAAGAAGCTGATCGGCG |
| | | AGGTG |
| | | 1 of 4 overlapping oligos creating a |
| | | novel trimerization domain, two |
| | | protease recognition sites, FLAG and |
| | | 6HIS tags (SEQ ID NO: 21) |
| | 14 | CTCGAGTGGCATGCATTTGGTACCGTGTTTTATATAA |
| | | CTATATCAATGATGATGATGATGATGCCCCCTTCCCT |
| | | CGATCCCCTTGTCATCGTCATCTTTATAATCCACCTC |
| | | GCCGATCAGC |
| | | 1 of 4 overlapping oligos creating a |
| | | novel trimerization domain, two |
| | | protease recognition sites, FLAG and |
| | | 6HIS tags (SEQ ID NO: 21) |
| | 15 | CCAGCATCAGCCAGGTGAAC |
| | | amplifying primer at the left end of |
| | | the novel sequence |
| | 16 | CTCGAGTGGCATGCATTTGG |
| | | amplifying primer at the right end of |
| | | the novel sequence that contains XhoI |
| | | site |
| | 17 | AACTACATCGACAAGCAGCTGCTG |
| | | the left end amplifying primer of the |
| | | final PCR product containing C |
| | | terminus fragment of the F gene and |
| | | the novel sequence |

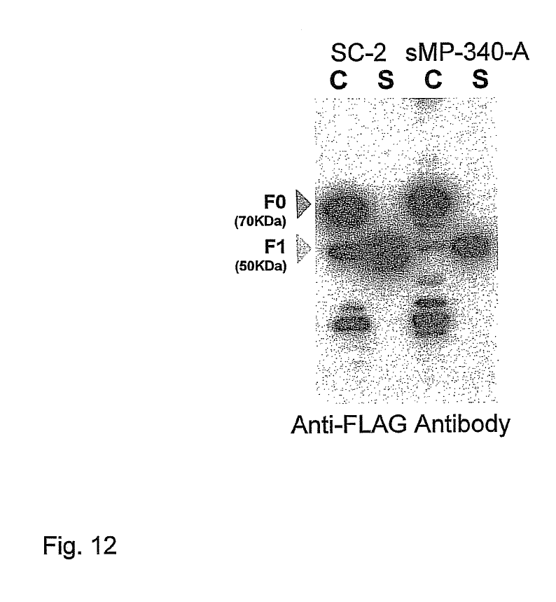

The sF proteins were produced by transfecting human embryonic kidney 293T cells that had been passaged twice over the previous two days and grown in medium lacking antibiotics. Cells were transfected with each DNA construct mixed with the transfection reagent TransIT (Mims, Corp.), as described in the manufacturer's instructions. After 48 hours of incubation at 37° C. in 5% CO2, the medium was harvested, centrifuged at low speed (2,000×g) to remove cell debris, and the supernatant and cell lysate were analyzed by western blot (FIG. 12). All three forms contain the two natural furin cleavage sites in the sF0 and were efficiently cleaved and released from the cells as expected (“S” lanes in FIG. 12). 80% to 90% of the sF protein produced in these cells was secreted as the fully cleaved sF protein, as described in the figure legend.

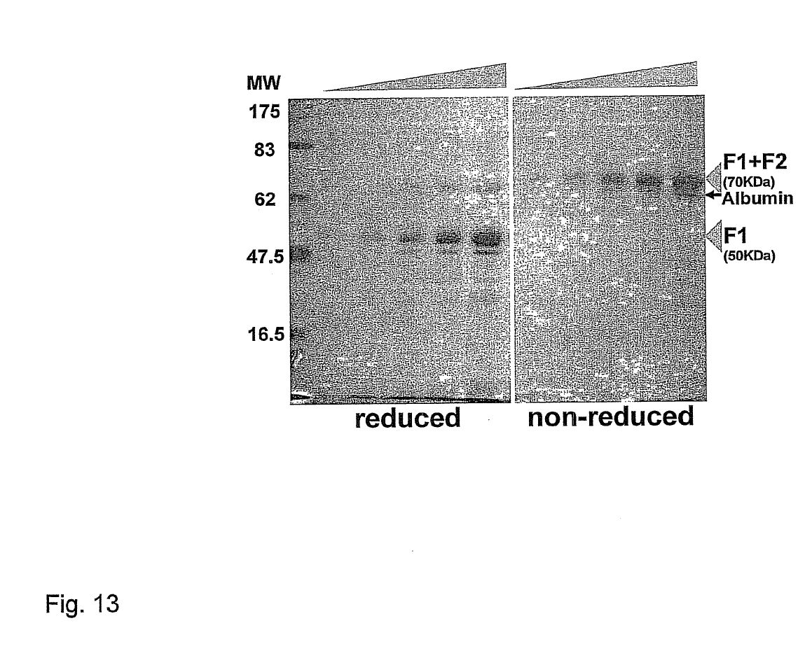

To generate a larger amount of purified sF protein we repeated the protocol described above in 3 150 mm tissue culture dishes. At 48 hr post-transfection we collected the medium and purified the protein on a Nickel column (Qiagen), according to the manufacturer's instructions. The sF protein binds to the nickel column because of the 6HIS tag (SEQ ID NO: 21) and can be specifically eluted with imidizol. The purified sF protein was easily detected by SDS-PAGE and Coomassie blue staining (FIG. 13). The reduced sF protein migrated at 50 kDa, consistent with the sF1 molecule. The non-reduced sF protein migrated at 70 kDa, consistent with the sF1+F2 disulfide linked monomer. A minor contaminant at 66 kDa, probably albumin, was also detected. The sF protein preparations can be produced with even fewer contaminants by: 1) eliminating or greatly reducing the serum in the growth medium; 2) passing the sF protein over a Cibacron disk (Sigma-Aldrich) to remove the albumin; 3) purifying the sF protein in a second round on a fresh NNickel column; 4) purify the sF protein on a Chromium column; and 5) other methods. Enough sF protein was generated and purified by this method to be readily visible by Coomassie blue staining of a polyacrylamide gel (FIG. 13). We estimate that the total amount of partially purified protein produced by this method was 0.5 mg. A similar yield was obtained by a second harvest from the same plates at 72 hr post-transfection. The purified protein was stored at −20° C. before beginning the analysis.

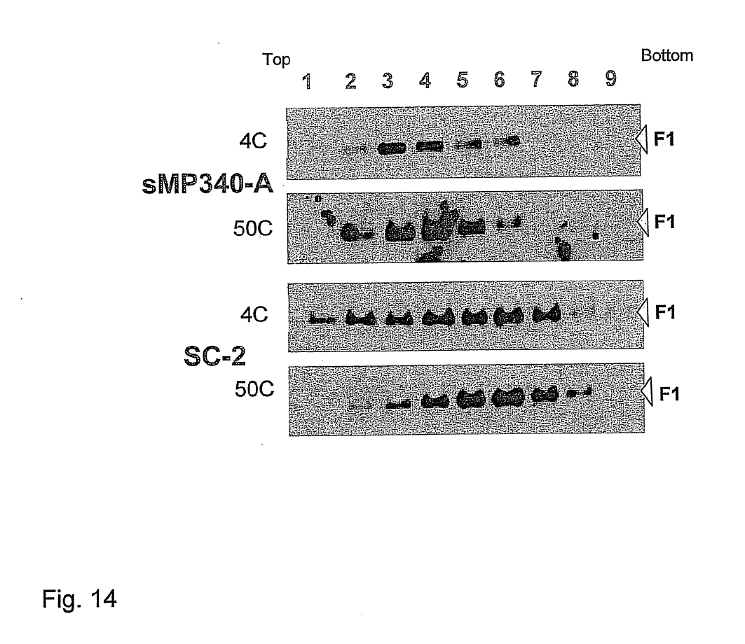

To determine whether the partially purified sF proteins that we had produced were in the pre-triggered or post-triggered form, we analyzed them by ultracentrifugation through linear sucrose gradients. The pre-triggered form should not migrate very far into the gradient, but the post-triggered form will aggregate via the hydrophobic fusion peptide that is exposed upon triggering and migrate further into the gradient, as found for the PIV5 sF protein (Connolly et al., 2006. Proc Natl Acad Sci USA 103(47): 17903-8). The 15-55% linear sucrose gradients were ultracentrifuged for 18 hours at 41,000 rpm in an SW41 rotor (Beckman). In our initial experiments, both SC-2 and sMP340-A migrated further into the sucrose gradients than expected (FIG. 14), indicating that they were aggregated. We had expected SC-2 to migrate in this manner, indicative of aggregation, but not sMP340-A. We hypothesized that freezing the protein between the time of production and purification and the sucrose gradient might be responsible for the sMP340-A migration indicating aggregation.

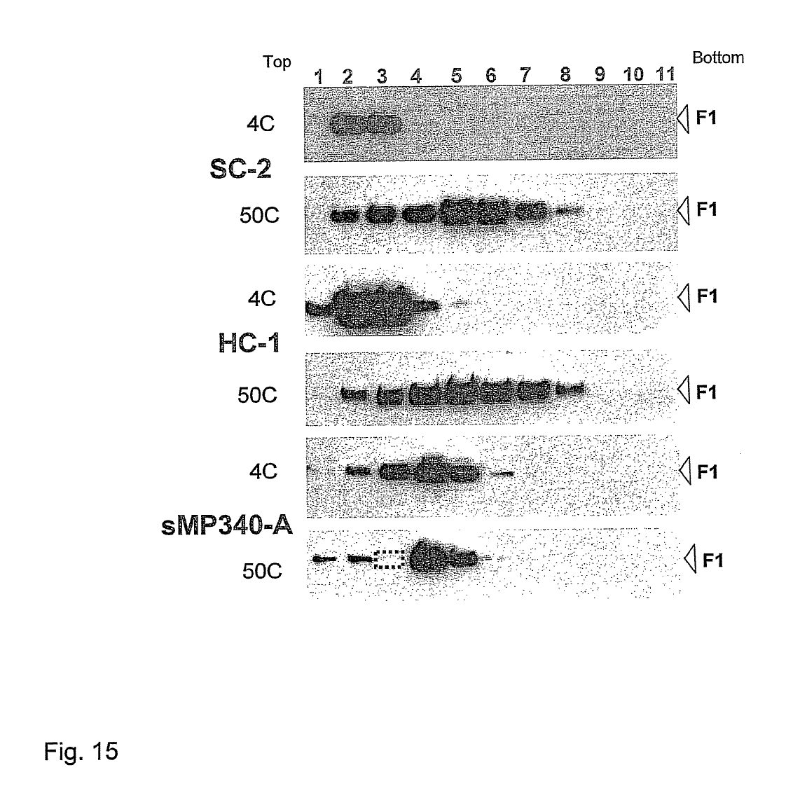

In the next attempt, we performed the complete experiment without freezing the sF proteins. The sMP340-A sF protein again did not remain at the top of the sucrose gradient nor did it move further into the gradient after treatment at 50° C., suggesting that this protein is not in the pre-triggered form to begin with and could not be triggered (FIG. 15). It is possible that the GCNt trimerization domain distorts the RSV sF protein. However, it is also possible that the GCNt domain that we added to the sF sequence was not in the proper phase with the HR2 domain, resulting in a distorted protein.

However, when we performed the sucrose gradient analysis without freezing the SC-2 sF protein, it remained near the top of the gradient (4° C. in FIG. 15). The RSV sF protein migration in this gradient is, therefore, indicative of the pre-triggered form.

In an attempt to trigger the SC-2 sF protein, we heated it at 50° C. for 1 hour and then analyzed it by velocity linear sucrose gradient centrifugation as described above. The heated sF migrated much further into the gradient (50° C. in FIG. 15), indicating that it had aggregated and, therefore, had been triggered.

The HC-1 sF protein behaved just like the SC-2 sF protein (FIG. 15), demonstrating that it, too, is a pre-triggered sF protein that can be triggered by heat. In another experiment we found that approximately 50% of the SC-2 sF protein is still in its pre-triggered state after storage at 4° C. for 3 weeks. We have also found that snap freezing the SC-2 sF protein on dry ice and storage at −80° C. or in liquid nitrogen maintains the pre-triggered state. The HC-1 sF protein is a novel paramyxoviral sF protein because of its potential for disulfide linkage of the C termini of the trimers. It is predicted to have the benefit of being even more stable than the SC-2 sF protein. Stability is an important characteristic for a reagent used in high throughput screening.

We produced two sF protein constructs, SC-2 and HC-1, that are in a pre-triggered form and can subsequently be triggered by the simplest known method, the addition of mild heat or cold. In some embodiments, the heat applied to induce triggering is from 37° C. to 55° C., including, for example, 42° C., 46° C. and 50° C., for a period of between 15 minutes to 4 hours, including, for example, 30 minutes, 45 minutes, 1 hour, 2 hours, and 3 hours. In one embodiment heating is performed at 50° C. for 1 hour. In another embodiment, triggering is caused by slow cooling, for example by placing the protein in an ice bath until it reaches 4° C. In other embodiments, triggering is obtained by placing the protein in a refrigerated environment, for example of 0° C., −4° C., −10° C., −15° C. or −20° C. until frozen.

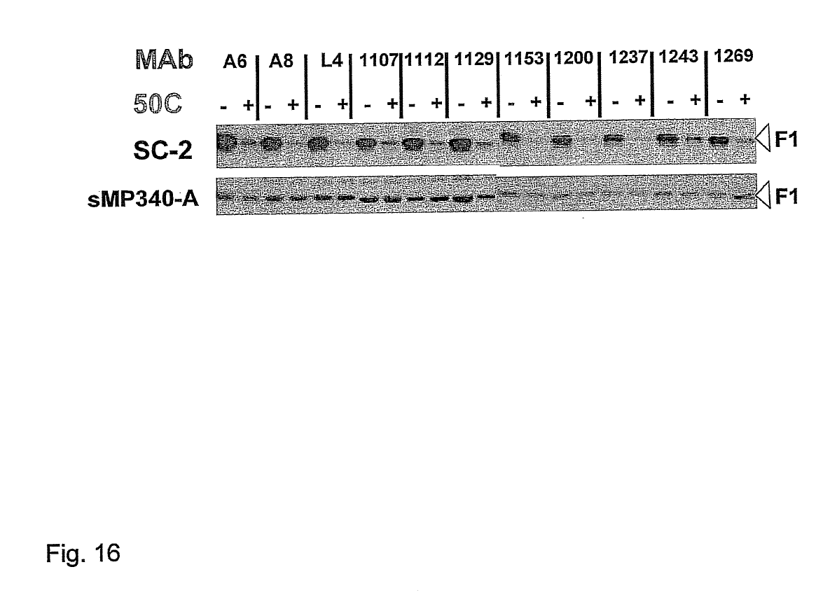

Confirming the pre-triggered state by reactivity with neutralizing antibodies. We confirmed the pre-triggered status of the constructs by performing antibody reactivity studies. According to our hypothesis, any mouse monoclonal antibody (MAb) against the F protein that neutralizes RSV infectivity in cell culture would bind to the virion form of the F protein and probably to the pre-triggered form of the F protein. If the SC-2 or sMP340-A protein represents the pre-triggered form of the sF protein, neutralizing MAbs should recognize it. To test this possibility, cells were transfected with plasmids expressing the SC-2 and sMP340-A sF proteins and metabolically labeled with ³⁵S-Metionine/Cysteine. Medium from these radiolabeled cells was immunoprecipitated with all 11 neutralizing MAbs (Crowe et al. 1998. Virology 252:373-5; Walsh, 1998 J Gen Virol. 1998 March; 79 (Pt 3):479-87; Walsh, E. E., and J. Hruska. 1983. J Virol 47:171-7) available to us (FIG. 16). Different MAbs in this group recognize at least 3 antigenic sites (Beeler, J. A., and K. van Wyke Coelingh. 1989. J Virol 63:2941-50). All 11 of these MAbs immunoprecipitated the SC-2 sF protein efficiently (FIG. 16, “-” lanes) suggesting that this sF protein is in the native F protein conformation. The same 11 MAbs did not immunoprecipitate the sMP340-A sF protein efficiently, suggesting that sMP340-A may not be in the native conformation.

MATERIALS & METHODS: Mouse MAbs A6, A8 and L4 against the RSV F protein were provided by Edward Walsh. Each of these MAbs binds to a different site on the RSV F protein. MAb L4 has been shown to neutralized RSV in the absence of complement, but it is 4-fold more effective in the presence of complement (Walsh, et al. 1986. J Gen Virol 67:505-13; Walsh, E. E., and J. Hruska. 1983. J Virol 47:171-7). The ability of a MAb to neutralize RSV indicates that it binds to the RSV virion, most likely the pre-triggered form, and blocks its ability to function in membrane fusion.

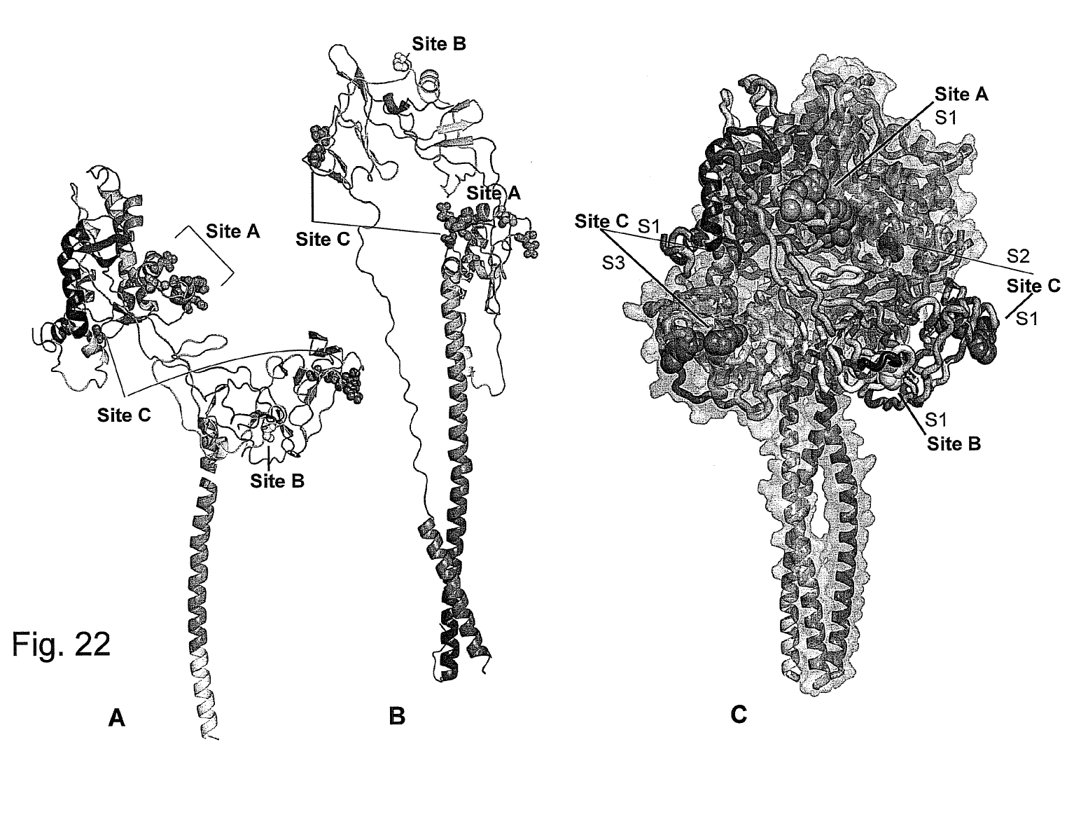

The remaining MAbs listed in FIG. 16 are also against the RSV F protein and were provided by Peter Collins and Judith Beeler, through the World Health Organization Paramyxovirus Reagent Bank. All of these MAbs neutralize RSV in the presence of complement (Beeler, J. A., and K. van Wyke Coelingh. 1989. J Virol 63:2941-50). They were organized into four major groups by competition for binding to the F protein: Group A (1153, 12000, 1237 and 1129); Group AB (1107); Group B (1112 and 1269); and Group C (1243) (Beeler, et al. 1989. J Virol 63:2941-50). MAb-resistant mutants for Groups A, B and C were selected by growing RSV in the presence of most of these antibodies. The rare survivors were amplified by growing the virus in culture and their F gene was sequenced (Crowe et al. 1998. Virology 252:373-5). The mutation sites are plotted on our F protein monomer models and the pre-triggered trimer (FIG. 22). MAb 1129 was later “humanized” and is presently used prophylactically to prevent and ameliorate RSV disease in the most vulnerable group, premature infants.

To determine the temperature at which the sF protein is triggered, the SC-2 sF protein was incubated at 4° C., 37° C., 42° C., 46° C., and 50° C. for an hour (FIG. 23). Loss of the pre-triggered state was determined by assessing the ability of MAb 1243 to bind to heat-treated sF protein. Some of the pre-triggered sF protein that was detected after the 4° C. incubation was lost after 37° C. incubation, and progressively more was lost after incubation at each higher temperature. The maximal loss of MAb reactivity followed incubation at 50° C.

The shape of the pre-triggered PIV5 sF protein changes upon triggering with mild heat, as determined by others, using electron microscopy. If mild heat also causes the SC-2 sF protein to trigger, as suggested above by its more rapid migration in velocity sucrose gradients (FIG. 15), this change should cause the loss of one or more of the epitopes recognized by the MAbs. To test this possibility, radiolabeled SC-2 sF protein was heated at 50° C. for an hour before being immunoprecipitated with each of the 11 neutralizing MAbs. The heated SC-2 sF protein lost its ability to be recognized efficiently by all 11 of the MAbs (FIG. 16, “+” lanes), indicating that heating had caused major conformational changes in the SC-2 sF protein, consistent with it being triggered by the heat treatment. Heating the sMP340-A sF protein had no effect on MAb binding (FIG. 16 “+” lanes), indicating that sMP340-A is not triggered by mild heat.

Because the SC-2 sF protein, lacking the transmembrane and cytoplasmic domains of the RSV F protein, is secreted in the pre-triggered form, detectable with 11 neutralizing MAbs, and can be triggered with mild heating to aggregate and at the same time to lose its MAb reactivity, the SC-2 sF protein is in the pre-triggered form. Therefore, the membrane anchor is not necessary to maintain the RSV sF protein in its pre-triggered form.

Triggering and stable association of the RSV sF protein with pure lipids in the form of liposomes. The classic method for detecting viral fusion protein triggering is to mix the protein with liposomes, artificial vesicles composed of pure lipids, and add a triggering agent. Triggering the viral protein exposes the fusion peptide which inserts into the nearest hydrophobic environment, the liposome membrane. Association is demonstrated by co-floatation in a sucrose gradient: the sF protein/liposome mixture is placed at the bottom of the tube in dense sucrose with progressively less dense sucrose layered above it, and centrifuged. Because of the low density of lipids, the liposomes float up through the gradient carrying associated proteins with them, while proteins that did not associate with the liposomes remain at the bottom of the gradient.

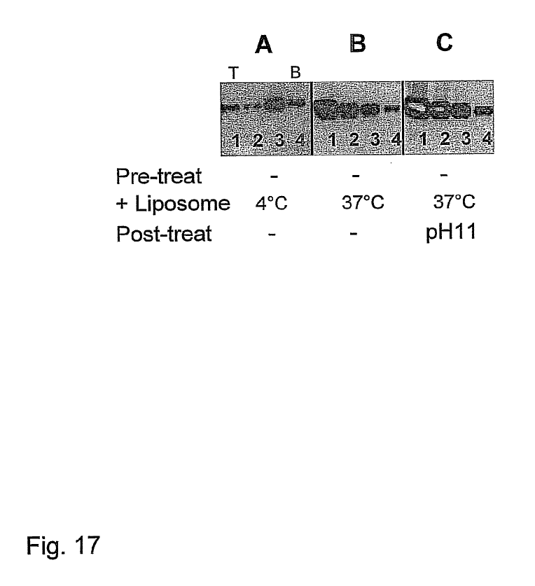

We used liposomes containing three types of lipids: POPC:POPE:Cholesterol=8:2:5 molar ratio (POPC is 1-Palmitoyl-2-Oleoyl-sn-Glycero-3-Phosphocholine; POPE is 1-Palmitoyl-2-Oleoyl-sn-Glycero-3-Phosphoethanol amine) to roughly model the content of the plasma membrane. Surprisingly, we found that the mere addition of the RSV sF protein to these liposomes and incubation at 37° C. caused liposome association and co-flotation (FIG. 17B). Longer, up to 6 hr, and shorter, 30 min, incubation led to a similar pattern. Treatment with pH 11 did not separate the RSV sF protein from the liposomes (FIG. 17C), confirming insertion of the fusion peptide into the liposome membranes. Interestingly, sF protein mixed with liposomes and incubated at 4° C. did not co-float efficiently (FIG. 17A), consistent with a requirement for lipid fluidity for triggering the RSV sF protein. These results are consistent with the fact that the RSV F protein does not require its attachment protein to be triggered. Theses results are also consistent with our hypothesis that the interaction between the sF protein CRAC1 domain and lipids of a target membrane triggers the sF protein.

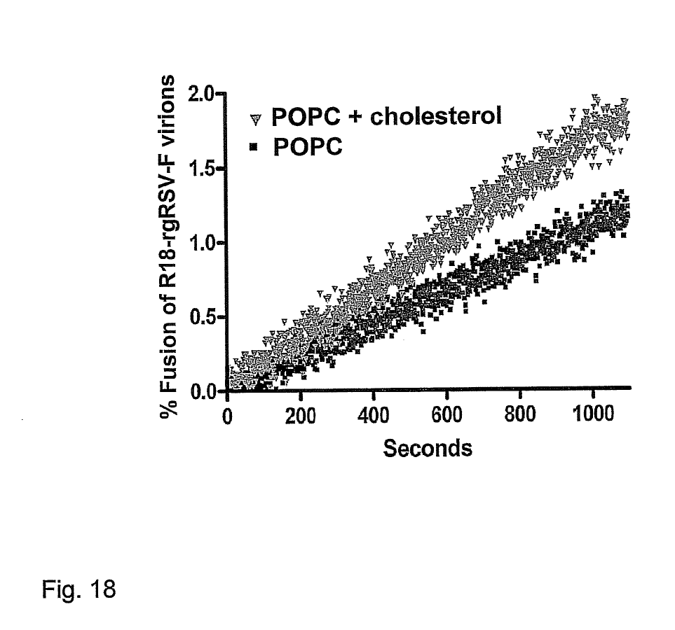

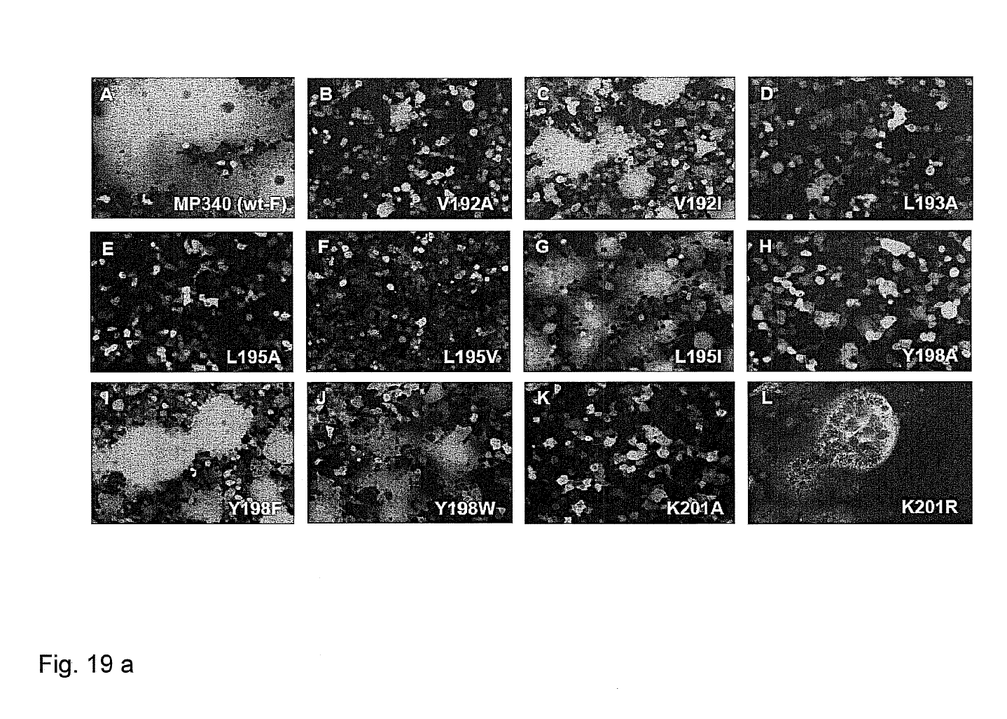

RSV virion fusion with pure lipids in the form of liposomes. We have examined the ability of sucrose density gradient-purified, recombinant green fluorescent protein-expressing virions with the F protein as their only glycoprotein (rgRSV-F), labeled with self-quenching amounts of R18 lipid dye, to fuse with liposomes prepared from pure lipids, leading to R18 dilution and fluorescence. 100% is defined as the fluorescence of the same amount of virions treated with the detergent Triton-X100 to dissociate and therefore dequench all of the R18. Over the course of 18 min, 1.2% of the rgRSV-F virions fused with POPC (1-Palmitoyl-2-Oleoyl-sn-Glycero-3-Phosphocholine) liposomes (FIG. 18), indicating again that the F protein, alone, is capable of causing membrane fusion, and that the F protein is triggered to perform its fusion function by interacting with lipids in the target cell membrane, in the absence of protein.

In this experiment, fusion of the virions with liposomes was not as rapid as sF protein triggering by liposomes (FIG. 18) in which essentially all of the sF protein was triggered following a 30 min incubation with liposomes. This difference in kinetics is probably due to the need for the concerted triggering of multiple F trimers at one time to form a fusion pore. Triggering of individual sF trimers would not require this kind of cooperativity. Furthermore, there are probably a multitude of virion particles for every infectious virion, perhaps partially due to prematurely triggered F proteins and/or the difficulty in triggering multiple F protein trimers simultaneously in a local area in order to initiate fusion pore formation. Therefore, -many triggering events may be necessary before the required organization is achieved.

The rgRSV-F virions fused more efficiently with liposomes containing 30% cholesterol in addition to POPC lipids: 1.7% over 18 min as compared to 1.2% of the liposomes lacking cholesterol (FIG. 18). This result suggests that cholesterol in the target membrane facilitates F protein-mediated viral fusion. However, cholesterol in the target liposome membrane was not required for virion fusion since virions also fused with liposomes lacking cholesterol.

### Example 3

### The CRAC Motif and the F Protein Triggering Mechanism

Experimental test of the role of the CRAC1 domain in fusion. Our discovery of the cholesterol-binding CRAC domain (CRAC1) lining the central pore in the crown of the RSV F protein in combination with our recognition of stabilizing interactions that would be disrupted if the CRAC1 domain were pulled away led us to discover that cholesterol is involved in the triggering mechanism. Both the CRAC1 domain and the interacting peptide are located within the region of the F protein that must reform into a long α-helix as the F protein is triggered. According to this discovery, mutation of any one of the three signature CRAC domain amino acids (V/L, X1-5, Y, X1-5, R/K) would have a major negative effect on fusion. The CRAC1 sequence in the RSV F protein is: 192VLDLKNYIDK. (SEQ ID NO. 20) The residue numbering corresponds to the amino acid sequence of SEQ ID NO: 1. The amino acids that compose the minimal CRAC domain are L195, Y198 and K201 (underlined). We hypothesized that mutating these signature amino acids to alanine, the simplest amino acid, should reduce the fusion activity of the F protein without changing the secondary structure, the α-helix. On the other hand, mutation of these amino acids to the alternate acceptable amino acid (i.e. L to V, or K to R) should have minimal effect on fusion.

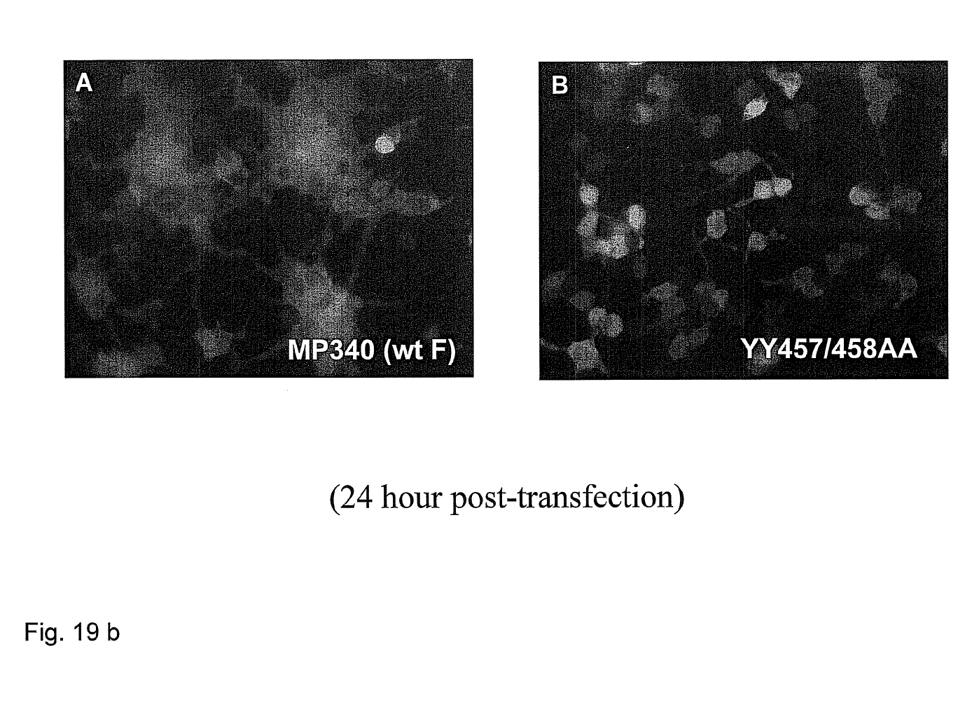

We made these mutations in the RSV F protein and tested their effects on cell-cell fusion (FIG. 19). As predicted, mutating the CRAC amino acids, L195, Y198 and K201, to alanine all severely inhibited fusion (FIG. 19a E,H,K). Also, as predicted, mutating the critical K201 CRAC amino acid to arginine, the alternate acceptable amino acid, had little effect on fusion (FIG. 19a L). Mutating L195 to valine should not affect fusion, however, this mutation destroyed F protein fusion (FIG. 19a F). L195 is clearly an important amino acid since changing it to either alanine or valine destroyed fusion. It is possible that valine cannot substitute for L195 in this particular CRAC sequence. Mutation of L195 to isoleucine did not inhibit fusion (FIG. 19a G). Although isoleucine has not been defined as a CRAC amino acid, it is very similar to leucine and so might be an acceptable replacement. For this reason, we have included isoleucine as a potential amino acid in the first CRAC position.

Alternatively, it is possible that either V192 or L193 may be the critical CRAC amino acid in the first position, instead of L195. Mutations that change V192 or L193 to alanine destroyed the fusion activity of the F protein (FIG. 19a B,D), indicating that one or both of these amino acids may indeed be the important CRAC amino acid in the first position instead of L195, or perhaps in addition to it. Again, replacement of V192 with isoleucine did not block fusion (FIG. 19a C), even though isoleucine is not included in the original CRAC definition. Nevertheless, isoleucine shares properties with valine and might be expected to be able to replace it.

Conservation of the trigger domain in the F1 protein: the role of CRAC1 in other viruses: CRAC1 domain is conserved among several paramyxoviruses, including human RSV, bovine RSV, and human metapneumovirus (FIG. 20), if phenylalanine (F) is substituted for the central tyrosine (Y) in the CRAC motif. This conservation among other similar viruses confirms our finding that CRAC1 is important for the F protein to perform its fusion function. The substitution of phenylalanine for tyrosine is predictable since this is a conservative amino acid change: both amino acids contain phenyl ring.

To directly test whether phenylalanine can substitute for tyrosine in the central CRAC1 position, we mutated Y198 to phenylalanine. This mutation did not greatly affect cell-cell fusion (FIG. 19a I), confirming that phenyalanine at this position allows the F protein to function. We also replaced Y198 with the other phenyl ring containing amino acid, tryptophan. This mutant also functioned in fusion (FIG. 19a J). Together, this data indicates that the central CRAC1 amino acid can be any phenyl ring containing amino acid: tyrosine, phenylalanine or tryptophan.

CRAC1 is also present at the same position in the F protein of parainfluenzavirus type 1 and parainfluenzavirus type 3, and shifted 5 amino acids toward the C terminus in the F protein of Newcastle disease virus. Nipah virus has a CRAC domain immediately at the base of the fusion peptide, a more N terminal position than the others, that might perform a similar function. Both parainfluenza virus type 1 and Newcastle disease virus have phenylalanine as the central amino acid in their CRAC1 domains. We have shown above that phenylalanine can substitute for tyrosine in the central position, so these two CRAC1 domains are likely functional.

Measles virus has sequences similar to the CRAC domain in the position of the RSV CRAC1, but this domain ends with an acidic amino acid instead of a basic amino acid. We propose that such a domain also binds cholesterol since a charge may be the important aspect of this amino acid rather than the type of charge, positive or negative. Furthermore, in FIG. 20 the conserved CRAC1A motif of the RSV-related viruses ends in a basic amino acid, but in an acidic amino acid in all of the other F proteins. This high level of conservation strongly suggests that an acidic amino acid can substitute for a basic amino acid at this position.

There are other paramyxoviruses, such as mumps virus, parainfluenzavirus types 2 and 4, and SV5, that do not have a CRAC domain in the CRAC1 position (FIG. 20).

Experimental Test of the Role of CRAC3 in Fusion

We have shown that a single mutation in the central tyrosine of the triggering CRAC1 domain inhibits cell-to-cell fusion (FIG. 19a E). We have also mutated the central two tyrosines of the CRAC3 domain to alanines. This mutant F protein did not cause fusion by 24 hr (FIG. 19b A compared to wild-type F in panel B). However, small syncytia were apparent by 72 hr, suggesting that the CRAC3 domain greatly enhances the rate of fusion but is not absolutely essential for fusion.

TABLE 4
Model Coordinates
F2 model coordinantes (discloses SEQ ID NO: 42 twice)

| | | | | | | | | | | | | |
|---|---|---|---|---|---|---|---|---|---|---|---|---|
| ATOM | 1 | N | ILE | A | 28 | 91.809 | 240.017 | 19.294 | 1.00 | 50.00 | AAAB | N |
| ATOM | 2 | CA | ILE | A | 28 | 91.429 | 239.171 | 18.142 | 1.00 | 50.00 | AAAB | C |
| ATOM | 3 | C | ILE | A | 28 | 92.584 | 238.818 | 17.230 | 1.00 | 50.00 | AAAB | C |
| ATOM | 4 | O | ILE | A | 28 | 92.608 | 239.253 | 16.084 | 1.00 | 50.00 | AAAB | O |
| ATOM | 5 | CB | ILE | A | 28 | 90.337 | 239.866 | 17.312 | 1.00 | 50.00 | AAAB | C |
| ATOM | 6 | CG1 | ILE | A | 28 | 90.646 | 241.338 | 16.976 | 1.00 | 50.00 | AAAB | C |
| ATOM | 7 | CG2 | ILE | A | 28 | 88.995 | 239.718 | 18.024 | 1.00 | 50.00 | AAAB | C |
| ATOM | 8 | CD1 | ILE | A | 28 | 89.882 | 241.836 | 15.747 | 1.00 | 50.00 | AAAB | C |
| ATOM | 9 | 1H | ILE | A | 28 | 90.973 | 240.175 | 19.891 | 0.00 | 0.00 | AAAB | H |
| ATOM | 10 | 2H | ILE | A | 28 | 92.170 | 240.933 | 18.959 | 0.00 | 0.00 | AAAB | H |
| ATOM | 11 | 3H | ILE | A | 28 | 92.556 | 239.548 | 19.846 | 0.00 | 0.00 | AAAB | H |
| ATOM | 12 | N | THR | A | 29 | 93.579 | 238.055 | 17.774 | 1.00 | 50.00 | AAAB | N |
| ATOM | 13 | CA | THR | A | 29 | 94.849 | 237.970 | 17.035 | 1.00 | 50.00 | AAAB | C |
| ATOM | 14 | C | THR | A | 29 | 95.255 | 239.405 | 16.869 | 1.00 | 50.00 | AAAB | C |
| ATOM | 15 | O | THR | A | 29 | 95.303 | 239.942 | 15.768 | 1.00 | 50.00 | AAAB | O |
| ATOM | 16 | CB | THR | A | 29 | 94.653 | 237.213 | 15.692 | 1.00 | 50.00 | AAAB | C |
| ATOM | 17 | CG2 | THR | A | 29 | 95.704 | 237.354 | 14.584 | 1.00 | 50.00 | AAAB | C |
| ATOM | 18 | OG1 | THR | A | 29 | 94.493 | 235.830 | 15.979 | 1.00 | 50.00 | AAAB | O |
| ATOM | 19 | H | THR | A | 29 | 93.540 | 237.755 | 18.726 | 0.00 | 0.00 | AAAB | H |
| ATOM | 20 | HG1 | THR | A | 29 | 94.519 | 235.413 | 15.129 | 0.00 | 0.00 | AAAB | H |
| ATOM | 21 | N | GLU | A | 30 | 95.363 | 240.034 | 18.083 | 1.00 | 50.00 | AAAB | N |
| ATOM | 22 | CA | GLU | A | 30 | 95.379 | 241.492 | 18.294 | 1.00 | 50.00 | AAAB | C |
| ATOM | 23 | C | GLU | A | 30 | 96.174 | 241.998 | 17.135 | 1.00 | 50.00 | AAAB | C |
| ATOM | 24 | O | GLU | A | 30 | 97.090 | 241.262 | 16.851 | 1.00 | 50.00 | AAAB | O |
| ATOM | 25 | CB | GLU | A | 30 | 96.166 | 241.616 | 19.587 | 1.00 | 50.00 | AAAB | C |
| ATOM | 26 | CG | GLU | A | 30 | 95.704 | 242.501 | 20.743 | 1.00 | 50.00 | AAAB | C |
| ATOM | 27 | CD | GLU | A | 30 | 95.719 | 243.977 | 20.395 | 1.00 | 50.00 | AAAB | C |
| ATOM | 28 | OE1 | GLU | A | 30 | 96.469 | 244.393 | 19.503 | 1.00 | 50.00 | AAAB | O |
| ATOM | 29 | OE2 | GLU | A | 30 | 94.956 | 244.709 | 21.024 | 1.00 | 50.00 | AAAB | O |
| ATOM | 30 | H | GLU | A | 30 | 95.472 | 239.462 | 18.894 | 0.00 | 0.00 | AAAB | H |
| ATOM | 31 | N | GLU | A | 31 | 95.850 | 243.075 | 16.411 | 1.00 | 50.00 | AAAB | N |
| ATOM | 32 | CA | GLU | A | 31 | 96.827 | 243.068 | 15.362 | 1.00 | 50.00 | AAAB | C |
| ATOM | 33 | C | GLU | A | 31 | 98.074 | 243.921 | 15.454 | 1.00 | 50.00 | AAAB | C |
| ATOM | 34 | O | GLU | A | 31 | 98.371 | 243.987 | 14.282 | 1.00 | 50.00 | AAAB | O |
| ATOM | 35 | CB | GLU | A | 31 | 96.266 | 242.894 | 13.888 | 1.00 | 50.00 | AAAB | C |
| ATOM | 36 | CG | GLU | A | 31 | 95.775 | 244.146 | 13.112 | 1.00 | 50.00 | AAAB | C |
| ATOM | 37 | CD | GLU | A | 31 | 96.331 | 244.302 | 11.679 | 1.00 | 50.00 | AAAB | C |
| ATOM | 38 | OE1 | GLU | A | 31 | 95.507 | 244.387 | 10.774 | 1.00 | 50.00 | AAAB | O |
| ATOM | 39 | OE2 | GLU | A | 31 | 97.545 | 244.373 | 11.465 | 1.00 | 50.00 | AAAB | O |
| ATOM | 40 | H | GLU | A | 31 | 95.011 | 243.592 | 16.322 | 0.00 | 0.00 | AAAB | H |
| ATOM | 41 | N | PHE | A | 32 | 98.810 | 244.344 | 16.680 | 1.00 | 50.00 | AAAB | N |
| ATOM | 42 | CA | PHE | A | 32 | 100.311 | 244.598 | 17.252 | 1.00 | 50.00 | AAAB | C |
| ATOM | 43 | C | PHE | A | 32 | 101.725 | 243.635 | 17.489 | 1.00 | 50.00 | AAAB | C |
| ATOM | 44 | O | PHE | A | 32 | 102.187 | 244.010 | 18.542 | 1.00 | 50.00 | AAAB | O |
| ATOM | 45 | CB | PHE | A | 32 | 100.162 | 245.442 | 18.523 | 1.00 | 50.00 | AAAB | C |
| ATOM | 46 | CG | PHE | A | 32 | 99.694 | 246.715 | 17.952 | 1.00 | 50.00 | AAAB | C |
| ATOM | 47 | CD1 | PHE | A | 32 | 98.315 | 246.976 | 17.884 | 1.00 | 50.00 | AAAB | C |
| ATOM | 48 | CD2 | PHE | A | 32 | 100.661 | 247.525 | 17.340 | 1.00 | 50.00 | AAAB | C |
| ATOM | 49 | CE1 | PHE | A | 32 | 97.878 | 247.930 | 16.960 | 1.00 | 50.00 | AAAB | C |
| ATOM | 50 | CE2 | PHE | A | 32 | 100.229 | 248.458 | 16.400 | 1.00 | 50.00 | AAAB | C |
| ATOM | 51 | CZ | PHE | A | 32 | 98.844 | 248.577 | 16.167 | 1.00 | 50.00 | AAAB | C |
| ATOM | 52 | H | PHE | A | 32 | 98.083 | 244.376 | 17.359 | 0.00 | 0.00 | AAAB | H |
| ATOM | 53 | N | TYR | A | 33 | 102.380 | 242.520 | 16.711 | 1.00 | 50.00 | AAAB | N |
| ATOM | 54 | CA | TYR | A | 33 | 103.101 | 241.110 | 16.689 | 1.00 | 50.00 | AAAB | C |
| ATOM | 55 | C | TYR | A | 33 | 104.084 | 241.086 | 15.478 | 1.00 | 50.00 | AAAB | C |
| ATOM | 56 | O | TYR | A | 33 | 104.550 | 240.051 | 15.001 | 1.00 | 50.00 | AAAB | O |
| ATOM | 57 | CB | TYR | A | 33 | 102.574 | 239.598 | 16.273 | 1.00 | 50.00 | AAAB | C |
| ATOM | 58 | CG | TYR | A | 33 | 102.482 | 238.189 | 17.014 | 1.00 | 50.00 | AAAB | C |
| ATOM | 59 | CD1 | TYR | A | 33 | 102.752 | 237.827 | 18.348 | 1.00 | 50.00 | AAAB | C |
| ATOM | 60 | CD2 | TYR | A | 33 | 102.026 | 237.088 | 16.271 | 1.00 | 50.00 | AAAB | C |
| ATOM | 61 | CE1 | TYR | A | 33 | 102.927 | 236.445 | 18.724 | 1.00 | 50.00 | AAAB | C |
| ATOM | 62 | CE2 | TYR | A | 33 | 101.955 | 235.780 | 16.845 | 1.00 | 50.00 | AAAB | C |
| ATOM | 63 | CZ | TYR | A | 33 | 102.706 | 235.185 | 18.007 | 1.00 | 50.00 | AAAB | C |
| ATOM | 64 | OH | TYR | A | 33 | 103.577 | 233.062 | 18.210 | 1.00 | 50.00 | AAAB | O |
| ATOM | 65 | H | TYR | A | 33 | 102.447 | 242.759 | 15.750 | 0.00 | 0.00 | AAAB | H |
| ATOM | 66 | HH | TYR | A | 33 | 104.206 | 232.671 | 17.619 | 0.00 | 0.00 | AAAB | H |
| ATOM | 67 | N | GLN | A | 34 | 104.415 | 242.285 | 15.006 | 1.00 | 50.00 | AAAB | N |
| ATOM | 68 | CA | GLN | A | 34 | 105.498 | 242.340 | 14.033 | 1.00 | 50.00 | AAAB | C |
| ATOM | 69 | C | GLN | A | 34 | 106.898 | 242.675 | 14.623 | 1.00 | 50.00 | AAAB | C |
| ATOM | 70 | O | GLN | A | 34 | 107.885 | 242.634 | 13.908 | 1.00 | 50.00 | AAAB | O |
| ATOM | 71 | CB | GLN | A | 34 | 105.009 | 243.237 | 12.871 | 1.00 | 50.00 | AAAB | C |
| ATOM | 72 | CG | GLN | A | 34 | 103.933 | 242.528 | 12.016 | 1.00 | 50.00 | AAAB | C |
| ATOM | 73 | CD | GLN | A | 34 | 103.024 | 243.485 | 11.263 | 1.00 | 50.00 | AAAB | C |
| ATOM | 74 | NE2 | GLN | A | 34 | 102.857 | 243.327 | 9.956 | 1.00 | 50.00 | AAAB | N |
| ATOM | 75 | OE1 | GLN | A | 34 | 102.444 | 244.357 | 11.848 | 1.00 | 50.00 | AAAB | O |
| ATOM | 76 | H | GLN | A | 34 | 103.938 | 243.099 | 15.313 | 0.00 | 0.00 | AAAB | H |
| ATOM | 77 | 1HE2 | GLN | A | 34 | 102.254 | 243.981 | 9.501 | 0.00 | 0.00 | AAAB | H |
| ATOM | 78 | 2HE2 | GLN | A | 34 | 103.297 | 242.607 | 9.436 | 0.00 | 0.00 | AAAB | H |
| ATOM | 79 | N | SER | A | 35 | 106.979 | 242.977 | 15.951 | 1.00 | 50.00 | AAAB | N |
| ATOM | 80 | CA | SER | A | 35 | 108.282 | 243.323 | 16.592 | 1.00 | 50.00 | AAAB | C |
| ATOM | 81 | C | SER | A | 35 | 109.008 | 242.202 | 17.356 | 1.00 | 50.00 | AAAB | C |
| ATOM | 82 | O | SER | A | 35 | 109.849 | 242.429 | 18.214 | 1.00 | 50.00 | AAAB | O |
| ATOM | 83 | CB | SER | A | 35 | 108.264 | 244.509 | 17.582 | 1.00 | 50.00 | AAAB | C |
| ATOM | 84 | OG | SER | A | 35 | 107.365 | 245.563 | 17.231 | 1.00 | 50.00 | AAAB | O |
| ATOM | 85 | H | SER | A | 35 | 106.100 | 243.032 | 16.432 | 0.00 | 0.00 | AAAB | H |
| ATOM | 86 | HG | SER | A | 35 | 106.773 | 245.712 | 17.960 | 0.00 | 0.00 | AAAB | H |
| ATOM | 87 | N | THR | A | 36 | 108.629 | 240.978 | 16.977 | 1.00 | 50.00 | AAAB | N |
| ATOM | 88 | CA | THR | A | 36 | 109.329 | 239.710 | 17.203 | 1.00 | 50.00 | AAAB | C |
| ATOM | 89 | C | THR | A | 36 | 109.227 | 239.001 | 15.850 | 1.00 | 50.00 | AAAB | C |
| ATOM | 90 | O | THR | A | 36 | 109.666 | 237.874 | 15.683 | 1.00 | 50.00 | AAAB | O |
| ATOM | 91 | CB | THR | A | 36 | 108.737 | 238.826 | 18.347 | 1.00 | 50.00 | AAAB | C |
| ATOM | 92 | CG2 | THR | A | 36 | 109.561 | 237.627 | 18.806 | 1.00 | 50.00 | AAAB | C |
| ATOM | 93 | OG1 | THR | A | 36 | 108.355 | 239.610 | 19.485 | 1.00 | 50.00 | AAAB | O |
| ATOM | 94 | H | THR | A | 36 | 107.788 | 240.955 | 16.435 | 0.00 | 0.00 | AAAB | H |
| ATOM | 95 | HG1 | THR | A | 36 | 107.628 | 239.137 | 19.885 | 0.00 | 0.00 | AAAB | H |
| ATOM | 96 | N | CYS | A | 37 | 108.687 | 239.779 | 14.864 | 1.00 | 50.00 | AAAB | N |
| ATOM | 97 | CA | CYS | A | 37 | 108.820 | 239.536 | 13.438 | 1.00 | 50.00 | AAAB | C |
| ATOM | 98 | C | CYS | A | 37 | 107.833 | 238.571 | 12.810 | 1.00 | 50.00 | AAAB | C |
| ATOM | 99 | O | CYS | A | 37 | 108.181 | 237.871 | 11.880 | 1.00 | 50.00 | AAAB | O |
| ATOM | 100 | CB | CYS | A | 37 | 110.281 | 239.253 | 13.069 | 1.00 | 50.00 | AAAB | C |
| ATOM | 101 | SG | CYS | A | 37 | 111.299 | 240.746 | 12.920 | 1.00 | 50.00 | AAAB | S |
| ATOM | 102 | H | CYS | A | 37 | 108.145 | 240.600 | 15.036 | 0.00 | 0.00 | AAAB | H |
| ATOM | 103 | N | SER | A | 38 | 106.586 | 238.587 | 13.312 | 1.00 | 50.00 | AAAB | N |
| ATOM | 104 | CA | SER | A | 38 | 105.590 | 237.872 | 12.517 | 1.00 | 50.00 | AAAB | C |
| ATOM | 105 | C | SER | A | 38 | 104.610 | 238.821 | 11.853 | 1.00 | 50.00 | AAAB | C |
| ATOM | 106 | O | SER | A | 38 | 103.841 | 239.504 | 12.514 | 1.00 | 50.00 | AAAB | O |
| ATOM | 107 | CB | SER | A | 38 | 104.843 | 236.852 | 13.377 | 1.00 | 50.00 | AAAB | C |
| ATOM | 108 | OG | SER | A | 38 | 104.005 | 236.041 | 12.546 | 1.00 | 50.00 | AAAB | O |
| ATOM | 109 | H | SER | A | 38 | 106.316 | 239.097 | 14.127 | 0.00 | 0.00 | AAAB | H |
| ATOM | 110 | HG | SER | A | 38 | 103.545 | 235.451 | 13.128 | 0.00 | 0.00 | AAAB | H |
| ATOM | 111 | N | ALA | A | 39 | 104.669 | 238.847 | 10.516 | 1.00 | 50.00 | AAAB | N |
| ATOM | 112 | CA | ALA | A | 39 | 103.738 | 239.742 | 9.860 | 1.00 | 50.00 | AAAB | C |
| ATOM | 113 | C | ALA | A | 39 | 102.653 | 239.079 | 9.071 | 1.00 | 50.00 | AAAB | C |
| ATOM | 114 | O | ALA | A | 39 | 102.845 | 238.089 | 8.382 | 1.00 | 50.00 | AAAB | O |
| ATOM | 115 | CB | ALA | A | 39 | 104.461 | 240.629 | 8.878 | 1.00 | 50.00 | AAAB | C |
| ATOM | 116 | H | ALA | A | 39 | 105.305 | 238.293 | 9.982 | 0.00 | 0.00 | AAAB | H |
| ATOM | 117 | N | VAL | A | 40 | 101.496 | 239.736 | 9.141 | 1.00 | 50.00 | AAAB | N |
| ATOM | 118 | CA | VAL | A | 40 | 100.468 | 239.262 | 8.237 | 1.00 | 50.00 | AAAB | C |
| ATOM | 119 | C | VAL | A | 40 | 100.635 | 239.712 | 6.788 | 1.00 | 50.00 | AAAB | C |
| ATOM | 120 | O | VAL | A | 40 | 100.578 | 240.887 | 6.443 | 1.00 | 50.00 | AAAB | O |
| ATOM | 121 | CB | VAL | A | 40 | 99.094 | 239.552 | 8.820 | 1.00 | 50.00 | AAAB | C |
| ATOM | 122 | CG1 | VAL | A | 40 | 98.751 | 241.042 | 8.979 | 1.00 | 50.00 | AAAB | C |
| ATOM | 123 | CG2 | VAL | A | 40 | 98.120 | 238.728 | 8.003 | 1.00 | 50.00 | AAAB | C |
| ATOM | 124 | H | VAL | A | 40 | 101.387 | 240.530 | 9.736 | 0.00 | 0.00 | AAAB | H |
| ATOM | 125 | N | SER | A | 41 | 100.876 | 238.676 | 5.966 | 1.00 | 50.00 | AAAB | N |
| ATOM | 126 | CA | SER | A | 41 | 101.047 | 238.921 | 4.540 | 1.00 | 50.00 | AAAB | C |
| ATOM | 127 | C | SER | A | 41 | 99.728 | 239.000 | 3.779 | 1.00 | 50.00 | AAAB | C |
| ATOM | 128 | O | SER | A | 41 | 99.589 | 239.723 | 2.800 | 1.00 | 50.00 | AAAB | O |
| ATOM | 129 | CB | SER | A | 41 | 101.985 | 237.856 | 3.957 | 1.00 | 50.00 | AAAB | C |
| ATOM | 130 | OG | SER | A | 41 | 102.426 | 238.215 | 2.641 | 1.00 | 50.00 | AAAB | O |
| ATOM | 131 | H | SER | A | 41 | 100.893 | 237.745 | 6.331 | 0.00 | 0.00 | AAAB | H |
| ATOM | 132 | HG | SER | A | 41 | 103.099 | 237.588 | 2.417 | 0.00 | 0.00 | AAAB | H |
| ATOM | 133 | N | LYS | A | 42 | 98.748 | 238.207 | 4.273 | 1.00 | 50.00 | AAAB | N |
| ATOM | 134 | CA | LYS | A | 42 | 97.481 | 238.116 | 3.534 | 1.00 | 50.00 | AAAB | C |
| ATOM | 135 | C | LYS | A | 42 | 96.279 | 237.930 | 4.443 | 1.00 | 50.00 | AAAB | C |
| ATOM | 136 | O | LYS | A | 42 | 96.350 | 237.264 | 5.466 | 1.00 | 50.00 | AAAB | O |
| ATOM | 137 | CB | LYS | A | 42 | 97.495 | 236.988 | 2.491 | 1.00 | 50.00 | AAAB | C |
| ATOM | 138 | CG | LYS | A | 42 | 98.507 | 237.126 | 1.346 | 1.00 | 50.00 | AAAB | C |
| ATOM | 139 | CD | LYS | A | 42 | 98.516 | 235.943 | 0.373 | 1.00 | 50.00 | AAAB | C |
| ATOM | 140 | CE | LYS | A | 42 | 97.188 | 235.741 | −0.359 | 1.00 | 50.00 | AAAB | C |
| ATOM | 141 | NZ | LYS | A | 42 | 96.915 | 236.926 | −1.184 | 1.00 | 50.00 | AAAB | N |
| ATOM | 142 | H | LYS | A | 42 | 98.895 | 237.697 | 5.123 | 0.00 | 0.00 | AAAB | H |
| ATOM | 143 | 1HZ | LYS | A | 42 | 96.030 | 236.798 | −1.713 | 0.00 | 0.00 | AAAB | H |
| ATOM | 144 | 2HZ | LYS | A | 42 | 97.701 | 237.059 | −1.853 | 0.00 | 0.00 | AAAB | H |
| ATOM | 145 | 3HZ | LYS | A | 42 | 96.846 | 237.765 | −0.573 | 0.00 | 0.00 | AAAB | H |
| ATOM | 146 | N | GLY | A | 43 | 95.172 | 238.562 | 4.005 | 1.00 | 50.00 | AAAB | N |
| ATOM | 147 | CA | GLY | A | 43 | 93.916 | 238.452 | 4.750 | 1.00 | 50.00 | AAAB | C |
| ATOM | 148 | C | GLY | A | 43 | 92.712 | 238.291 | 3.844 | 1.00 | 50.00 | AAAB | C |
| ATOM | 149 | O | GLY | A | 43 | 92.550 | 239.029 | 2.880 | 1.00 | 50.00 | AAAB | O |
| ATOM | 150 | H | GLY | A | 43 | 95.211 | 239.093 | 3.162 | 0.00 | 0.00 | AAAB | H |
| ATOM | 151 | N | TYR | A | 44 | 91.898 | 237.261 | 4.170 | 1.00 | 50.00 | AAAB | N |
| ATOM | 152 | CA | TYR | A | 44 | 91.119 | 236.624 | 3.111 | 1.00 | 50.00 | AAAB | C |
| ATOM | 153 | C | TYR | A | 44 | 89.633 | 236.162 | 3.569 | 1.00 | 50.00 | AAAB | C |
| ATOM | 154 | O | TYR | A | 44 | 89.467 | 236.088 | 4.779 | 1.00 | 50.00 | AAAB | O |
| ATOM | 155 | CB | TYR | A | 44 | 92.325 | 235.747 | 2.516 | 1.00 | 50.00 | AAAB | C |
| ATOM | 156 | CG | TYR | A | 44 | 92.841 | 234.360 | 3.192 | 1.00 | 50.00 | AAAB | C |
| ATOM | 157 | CD1 | TYR | A | 44 | 92.140 | 231.491 | 2.968 | 1.00 | 50.00 | AAAB | C |
| ATOM | 158 | CD2 | TYR | A | 44 | 94.143 | 234.410 | 4.261 | 1.00 | 50.00 | AAAB | C |
| ATOM | 159 | CE1 | TYR | A | 44 | 93.113 | 230.680 | 3.962 | 1.00 | 50.00 | AAAB | C |
| ATOM | 160 | CE2 | TYR | A | 44 | 94.735 | 233.011 | 4.962 | 1.00 | 50.00 | AAAB | C |
| ATOM | 161 | CZ | TYR | A | 44 | 94.227 | 231.432 | 4.885 | 1.00 | 50.00 | AAAB | C |
| ATOM | 162 | OH | TYR | A | 44 | 94.614 | 230.583 | 5.890 | 1.00 | 50.00 | AAAB | O |
| ATOM | 163 | H | TYR | A | 44 | 92.111 | 236.735 | 4.990 | 0.00 | 0.00 | AAAB | H |
| ATOM | 164 | HH | TYR | A | 44 | 94.717 | 231.021 | 6.719 | 0.00 | 0.00 | AAAB | H |
| ATOM | 165 | N | LEU | A | 45 | 88.557 | 235.910 | 2.675 | 1.00 | 50.00 | AAAB | N |
| ATOM | 166 | CA | LEU | A | 45 | 87.064 | 235.614 | 2.969 | 1.00 | 50.00 | AAAB | C |
| ATOM | 167 | C | LEU | A | 45 | 86.295 | 234.217 | 2.719 | 1.00 | 50.00 | AAAB | C |
| ATOM | 168 | O | LEU | A | 45 | 85.961 | 233.940 | 1.572 | 1.00 | 50.00 | AAAB | O |
| ATOM | 169 | CB | LEU | A | 45 | 86.201 | 236.520 | 2.118 | 1.00 | 50.00 | AAAB | C |
| ATOM | 170 | CG | LEU | A | 45 | 86.206 | 237.970 | 2.511 | 1.00 | 50.00 | AAAB | C |
| ATOM | 171 | CD1 | LEU | A | 45 | 85.248 | 238.739 | 1.603 | 1.00 | 50.00 | AAAB | C |
| ATOM | 172 | CD2 | LEU | A | 45 | 85.872 | 238.131 | 3.993 | 1.00 | 50.00 | AAAB | C |
| ATOM | 173 | H | LEU | A | 45 | 88.799 | 235.964 | 1.705 | 0.00 | 0.00 | AAAB | H |
| ATOM | 174 | N | SER | A | 46 | 86.093 | 233.296 | 3.747 | 1.00 | 50.00 | AAAB | N |
| ATOM | 175 | CA | SER | A | 46 | 86.187 | 231.826 | 3.427 | 1.00 | 50.00 | AAAB | C |
| ATOM | 176 | C | SER | A | 46 | 85.093 | 231.196 | 2.631 | 1.00 | 50.00 | AAAB | C |
| ATOM | 177 | O | SER | A | 46 | 83.996 | 231.705 | 2.679 | 1.00 | 50.00 | AAAB | O |
| ATOM | 178 | CB | SER | A | 46 | 86.656 | 230.979 | 4.644 | 1.00 | 50.00 | AAAB | C |
| ATOM | 179 | OG | SER | A | 46 | 86.902 | 229.603 | 4.298 | 1.00 | 50.00 | AAAB | O |
| ATOM | 180 | H | SER | A | 46 | 86.197 | 233.571 | 4.701 | 0.00 | 0.00 | AAAB | H |
| ATOM | 181 | HG | SER | A | 46 | 86.934 | 229.114 | 5.113 | 0.00 | 0.00 | AAAB | H |
| ATOM | 182 | N | ALA | A | 47 | 85.384 | 230.108 | 1.885 | 1.00 | 50.00 | AAAB | N |
| ATOM | 183 | CA | ALA | A | 47 | 84.189 | 229.587 | 1.231 | 1.00 | 50.00 | AAAB | C |
| ATOM | 184 | C | ALA | A | 47 | 84.015 | 228.105 | 1.139 | 1.00 | 50.00 | AAAB | C |
| ATOM | 185 | O | ALA | A | 47 | 84.935 | 227.310 | 0.982 | 1.00 | 50.00 | AAAB | O |
| ATOM | 186 | CB | ALA | A | 47 | 83.964 | 230.142 | −0.162 | 1.00 | 50.00 | AAAB | C |
| ATOM | 187 | H | ALA | A | 47 | 86.292 | 229.708 | 1.750 | 0.00 | 0.00 | AAAB | H |
| ATOM | 188 | N | LEU | A | 48 | 82.715 | 227.812 | 1.257 | 1.00 | 50.00 | AAAB | N |
| ATOM | 189 | CA | LEU | A | 48 | 82.234 | 226.447 | 1.250 | 1.00 | 50.00 | AAAB | C |
| ATOM | 190 | C | LEU | A | 48 | 81.446 | 226.138 | −0.009 | 1.00 | 50.00 | AAAB | C |
| ATOM | 191 | O | LEU | A | 48 | 80.847 | 226.993 | −0.654 | 1.00 | 50.00 | AAAB | O |
| ATOM | 192 | CB | LEU | A | 48 | 81.434 | 226.250 | 2.544 | 1.00 | 50.00 | AAAB | C |
| ATOM | 193 | CG | LEU | A | 48 | 81.009 | 224.830 | 2.929 | 1.00 | 50.00 | AAAB | C |
| ATOM | 194 | CD1 | LEU | A | 48 | 81.066 | 224.643 | 4.445 | 1.00 | 50.00 | AAAB | C |
| ATOM | 195 | CD2 | LEU | A | 48 | 79.625 | 224.457 | 2.397 | 1.00 | 50.00 | AAAB | C |
| ATOM | 196 | H | LEU | A | 48 | 82.065 | 228.562 | 1.381 | 0.00 | 0.00 | AAAB | H |
| ATOM | 197 | N | ARG | A | 49 | 81.498 | 224.833 | −0.301 | 1.00 | 50.00 | AAAB | N |
| ATOM | 198 | CA | ARG | A | 49 | 80.799 | 224.220 | −1.423 | 1.00 | 50.00 | AAAB | C |
| ATOM | 199 | C | ARG | A | 49 | 79.346 | 223.897 | −1.159 | 1.00 | 50.00 | AAAB | C |
| ATOM | 200 | O | ARG | A | 49 | 79.013 | 222.783 | −0.774 | 1.00 | 50.00 | AAAB | O |
| ATOM | 201 | CB | ARG | A | 49 | 81.519 | 222.923 | −1.768 | 1.00 | 50.00 | AAAB | C |
| ATOM | 202 | CG | ARG | A | 49 | 82.921 | 223.225 | −2.249 | 1.00 | 50.00 | AAAB | C |
| ATOM | 203 | CD | ARG | A | 49 | 82.775 | 224.072 | −3.498 | 1.00 | 50.00 | AAAB | C |
| ATOM | 204 | NE | ARG | A | 49 | 82.200 | 223.334 | −4.601 | 1.00 | 50.00 | AAAB | N |
| ATOM | 205 | CZ | ARG | A | 49 | 82.995 | 222.527 | −5.325 | 1.00 | 50.00 | AAAB | C |
| ATOM | 206 | NH1 | ARG | A | 49 | 84.328 | 222.564 | −5.227 | 1.00 | 50.00 | AAAB | N |
| ATOM | 207 | NH2 | ARG | A | 49 | 82.415 | 221.705 | −6.186 | 1.00 | 50.00 | AAAB | N |
| ATOM | 208 | H | ARG | A | 49 | 82.014 | 224.247 | 0.320 | 0.00 | 0.00 | AAAB | H |
| ATOM | 209 | HE | ARG | A | 49 | 81.220 | 223.383 | −4.783 | 0.00 | 0.00 | AAAB | H |
| ATOM | 210 | 1HH1 | ARG | A | 49 | 84.891 | 221.996 | −5.825 | 0.00 | 0.00 | AAAB | H |
| ATOM | 211 | 2HH1 | ARG | A | 49 | 84.779 | 223.169 | −4.568 | 0.00 | 0.00 | AAAB | H |
| ATOM | 212 | 1HH2 | ARG | A | 49 | 82.975 | 221.116 | −6.765 | 0.00 | 0.00 | AAAB | H |
| ATOM | 213 | 2HH2 | ARG | A | 49 | 81.418 | 221.704 | −6.257 | 0.00 | 0.00 | AAAB | H |
| ATOM | 214 | N | THR | A | 50 | 78.485 | 224.899 | −1.405 | 1.00 | 50.00 | AAAB | N |
| ATOM | 215 | CA | THR | A | 50 | 77.062 | 224.608 | −1.241 | 1.00 | 50.00 | AAAB | C |
| ATOM | 216 | C | THR | A | 50 | 76.521 | 223.573 | −2.219 | 1.00 | 50.00 | AAAB | C |
| ATOM | 217 | O | THR | A | 50 | 76.175 | 222.459 | −1.846 | 1.00 | 50.00 | AAAB | O |
| ATOM | 218 | CB | THR | A | 50 | 76.211 | 225.883 | −1.294 | 1.00 | 50.00 | AAAB | C |
| ATOM | 219 | CG2 | THR | A | 50 | 75.585 | 226.190 | 0.064 | 1.00 | 50.00 | AAAB | C |
| ATOM | 220 | OG1 | THR | A | 50 | 76.976 | 226.998 | −1.757 | 1.00 | 50.00 | AAAB | O |
| ATOM | 221 | H | THR | A | 50 | 78.799 | 225.794 | −1.726 | 0.00 | 0.00 | AAAB | H |
| ATOM | 222 | HG1 | THR | A | 50 | 76.360 | 227.683 | −1.983 | 0.00 | 0.00 | AAAB | H |
| ATOM | 223 | N | GLY | A | 51 | 76.486 | 224.002 | −3.493 | 1.00 | 50.00 | AAAB | N |
| ATOM | 224 | CA | GLY | A | 51 | 76.063 | 223.075 | −4.536 | 1.00 | 50.00 | AAAB | C |
| ATOM | 225 | C | GLY | A | 51 | 77.117 | 222.956 | −5.615 | 1.00 | 50.00 | AAAB | C |
| ATOM | 226 | O | GLY | A | 51 | 78.279 | 223.299 | −5.418 | 1.00 | 50.00 | AAAB | O |
| ATOM | 227 | H | GLY | A | 51 | 76.780 | 224.928 | −3.727 | 0.00 | 0.00 | AAAB | H |
| ATOM | 228 | N | TRP | A | 52 | 76.636 | 222.473 | −6.773 | 1.00 | 50.00 | AAAB | N |
| ATOM | 229 | CA | TRP | A | 52 | 77.518 | 222.273 | −7.921 | 1.00 | 50.00 | AAAB | C |
| ATOM | 230 | C | TRP | A | 52 | 76.712 | 222.018 | −9.177 | 1.00 | 50.00 | AAAB | C |
| ATOM | 231 | O | TRP | A | 52 | 75.650 | 221.408 | −9.143 | 1.00 | 50.00 | AAAB | O |
| ATOM | 232 | CB | TRP | A | 52 | 78.528 | 221.131 | −7.678 | 1.00 | 50.00 | AAAB | C |
| ATOM | 233 | CG | TRP | A | 52 | 77.832 | 219.802 | −7.445 | 1.00 | 50.00 | AAAB | C |
| ATOM | 234 | CD1 | TRP | A | 52 | 77.509 | 218.854 | −8.425 | 1.00 | 50.00 | AAAB | C |
| ATOM | 235 | CD2 | TRP | A | 52 | 77.346 | 219.232 | −6.208 | 1.00 | 50.00 | AAAB | C |
| ATOM | 236 | CE2 | TRP | A | 52 | 76.750 | 217.965 | −6.526 | 1.00 | 50.00 | AAAB | C |
| ATOM | 237 | CE3 | TRP | A | 52 | 77.360 | 219.680 | −4.870 | 1.00 | 50.00 | AAAB | C |
| ATOM | 238 | NE1 | TRP | A | 52 | 76.875 | 217.776 | −7.894 | 1.00 | 50.00 | AAAB | N |
| ATOM | 239 | CZ2 | TRP | A | 52 | 76.180 | 217.176 | −5.505 | 1.00 | 50.00 | AAAB | C |
| ATOM | 240 | CZ3 | TRP | A | 52 | 76.787 | 218.883 | −3.856 | 1.00 | 50.00 | AAAB | C |
| ATOM | 241 | CH2 | TRP | A | 52 | 76.199 | 217.638 | −4.173 | 1.00 | 50.00 | AAAB | C |
| ATOM | 242 | H | TRP | A | 52 | 75.668 | 222.219 | −6.836 | 0.00 | 0.00 | AAAB | H |
| ATOM | 243 | HE1 | TRP | A | 52 | 76.559 | 216.994 | −8.395 | 0.00 | 0.00 | AAAB | H |
| ATOM | 244 | N | TYR | A | 53 | 77.267 | 222.489 | −10.294 | 1.00 | 50.00 | AAAB | N |
| ATOM | 245 | CA | TYR | A | 53 | 76.655 | 222.066 | −11.544 | 1.00 | 50.00 | AAAB | C |
| ATOM | 246 | C | TYR | A | 53 | 77.514 | 221.044 | −12.263 | 1.00 | 50.00 | AAAB | C |
| ATOM | 247 | O | TYR | A | 53 | 78.716 | 220.941 | −12.046 | 1.00 | 50.00 | AAAB | O |
| ATOM | 248 | CB | TYR | A | 53 | 76.343 | 223.279 | −12.428 | 1.00 | 50.00 | AAAB | C |
| ATOM | 249 | CG | TYR | A | 53 | 77.618 | 223.949 | −12.872 | 1.00 | 50.00 | AAAB | C |
| ATOM | 250 | CD1 | TYR | A | 53 | 78.080 | 225.069 | −12.158 | 1.00 | 50.00 | AAAB | C |
| ATOM | 251 | CD2 | TYR | A | 53 | 78.302 | 223.420 | −13.984 | 1.00 | 50.00 | AAAB | C |
| ATOM | 252 | CE1 | TYR | A | 53 | 79.242 | 225.701 | −12.605 | 1.00 | 50.00 | AAAB | C |
| ATOM | 253 | CE2 | TYR | A | 53 | 79.478 | 224.037 | −14.407 | 1.00 | 50.00 | AAAB | C |
| ATOM | 254 | CZ | TYR | A | 53 | 79.903 | 225.204 | −13.746 | 1.00 | 50.00 | AAAB | C |
| ATOM | 255 | OH | TYR | A | 53 | 81.005 | 225.889 | −14.228 | 1.00 | 50.00 | AAAB | O |
| ATOM | 256 | H | TYR | A | 53 | 78.129 | 222.991 | −10.291 | 0.00 | 0.00 | AAAB | H |
| ATOM | 257 | HH | TYR | A | 53 | 81.680 | 225.283 | −14.509 | 0.00 | 0.00 | AAAB | H |
| ATOM | 258 | N | THR | A | 54 | 76.837 | 220.287 | −13.141 | 1.00 | 50.00 | AAAB | N |
| ATOM | 259 | CA | THR | A | 54 | 77.594 | 219.232 | −13.805 | 1.00 | 50.00 | AAAB | C |
| ATOM | 260 | C | THR | A | 54 | 77.357 | 219.171 | −15.300 | 1.00 | 50.00 | AAAB | C |
| ATOM | 261 | O | THR | A | 54 | 76.455 | 219.804 | −15.838 | 1.00 | 50.00 | AAAB | O |
| ATOM | 262 | CB | THR | A | 54 | 77.287 | 217.861 | −13.187 | 1.00 | 50.00 | AAAB | C |
| ATOM | 263 | CG2 | THR | A | 54 | 77.581 | 217.785 | −11.700 | 1.00 | 50.00 | AAAB | C |
| ATOM | 264 | OG1 | THR | A | 54 | 75.923 | 217.502 | −13.408 | 1.00 | 50.00 | AAAB | O |
| ATOM | 265 | H | THR | A | 54 | 75.858 | 220.406 | −13.303 | 0.00 | 0.00 | AAAB | H |
| ATOM | 266 | HG1 | THR | A | 54 | 75.831 | 216.650 | −13.008 | 0.00 | 0.00 | AAAB | H |
| ATOM | 267 | N | SER | A | 55 | 78.200 | 218.318 | −15.915 | 1.00 | 50.00 | AAAB | N |
| ATOM | 268 | CA | SER | A | 55 | 78.037 | 217.943 | −17.312 | 1.00 | 50.00 | AAAB | C |
| ATOM | 269 | C | SER | A | 55 | 78.749 | 216.619 | −17.553 | 1.00 | 50.00 | AAAB | C |
| ATOM | 270 | O | SER | A | 55 | 79.770 | 216.340 | −16.943 | 1.00 | 50.00 | AAAB | O |
| ATOM | 271 | CB | SER | A | 55 | 78.611 | 219.066 | −18.174 | 1.00 | 50.00 | AAAB | C |
| ATOM | 272 | OG | SER | A | 55 | 78.306 | 218.865 | −19.552 | 1.00 | 50.00 | AAAB | O |
| ATOM | 273 | H | SER | A | 55 | 79.005 | 217.976 | −15.432 | 0.00 | 0.00 | AAAB | H |
| ATOM | 274 | HG | SER | A | 55 | 78.581 | 219.657 | −19.998 | 0.00 | 0.00 | AAAB | H |
| ATOM | 275 | N | VAL | A | 56 | 78.174 | 215.799 | −18.443 | 1.00 | 50.00 | AAAB | N |
| ATOM | 276 | CA | VAL | A | 56 | 78.928 | 214.595 | −18.807 | 1.00 | 50.00 | AAAB | C |
| ATOM | 277 | C | VAL | A | 56 | 79.847 | 214.802 | −20.028 | 1.00 | 50.00 | AAAB | C |
| ATOM | 278 | O | VAL | A | 56 | 79.726 | 215.741 | −20.796 | 1.00 | 50.00 | AAAB | O |
| ATOM | 279 | CB | VAL | A | 56 | 78.009 | 213.313 | −18.785 | 1.00 | 50.00 | AAAB | C |
| ATOM | 280 | CG1 | VAL | A | 56 | 76.531 | 213.569 | −19.087 | 1.00 | 50.00 | AAAB | C |
| ATOM | 281 | CG2 | VAL | A | 56 | 78.503 | 212.091 | −19.564 | 1.00 | 50.00 | AAAB | C |
| ATOM | 282 | H | VAL | A | 56 | 77.344 | 216.107 | −18.908 | 0.00 | 0.00 | AAAB | H |
| ATOM | 283 | N | ILE | A | 57 | 80.845 | 213.924 | −20.142 | 1.00 | 50.00 | AAAB | N |
| ATOM | 284 | CA | ILE | A | 57 | 81.787 | 213.949 | −21.250 | 1.00 | 50.00 | AAAB | C |
| ATOM | 285 | C | ILE | A | 57 | 81.945 | 212.517 | −21.689 | 1.00 | 50.00 | AAAB | C |
| ATOM | 286 | O | ILE | A | 57 | 81.883 | 211.610 | −20.882 | 1.00 | 50.00 | AAAB | O |
| ATOM | 287 | CB | ILE | A | 57 | 83.120 | 214.556 | −20.766 | 1.00 | 50.00 | AAAB | C |
| ATOM | 288 | CG1 | ILE | A | 57 | 82.918 | 216.049 | −20.554 | 1.00 | 50.00 | AAAB | C |
| ATOM | 289 | CG2 | ILE | A | 57 | 84.282 | 214.344 | −21.744 | 1.00 | 50.00 | AAAB | C |
| ATOM | 290 | CD1 | ILE | A | 57 | 82.396 | 216.699 | −21.839 | 1.00 | 50.00 | AAAB | C |
| ATOM | 291 | H | ILE | A | 57 | 80.937 | 213.184 | −19.483 | 0.00 | 0.00 | AAAB | H |
| ATOM | 292 | N | THR | A | 58 | 82.137 | 212.332 | −22.990 | 1.00 | 50.00 | AAAB | N |
| ATOM | 293 | CA | THR | A | 58 | 82.369 | 210.975 | −23.443 | 1.00 | 50.00 | AAAB | C |
| ATOM | 294 | C | THR | A | 58 | 83.667 | 210.941 | −24.214 | 1.00 | 50.00 | AAAB | C |
| ATOM | 295 | O | THR | A | 58 | 83.746 | 211.367 | −25.356 | 1.00 | 50.00 | AAAB | O |
| ATOM | 296 | CB | THR | A | 58 | 81.182 | 210.530 | −24.298 | 1.00 | 50.00 | AAAB | C |
| ATOM | 297 | CG2 | THR | A | 58 | 79.954 | 210.187 | −23.463 | 1.00 | 50.00 | AAAB | C |
| ATOM | 298 | OG1 | THR | A | 58 | 80.840 | 211.567 | −25.219 | 1.00 | 50.00 | AAAB | O |
| ATOM | 299 | H | THR | A | 58 | 82.147 | 213.086 | −23.643 | 0.00 | 0.00 | AAAB | H |
| ATOM | 300 | HG1 | THR | A | 58 | 80.170 | 211.193 | −25.775 | 0.00 | 0.00 | AAAB | H |
| ATOM | 301 | N | ILE | A | 59 | 84.694 | 210.429 | −23.526 | 1.00 | 50.00 | AAAB | N |
| ATOM | 302 | CA | ILE | A | 59 | 85.977 | 210.312 | −24.199 | 1.00 | 50.00 | AAAB | C |
| ATOM | 303 | C | ILE | A | 59 | 86.054 | 209.095 | −25.117 | 1.00 | 50.00 | AAAB | C |
| ATOM | 304 | O | ILE | A | 59 | 86.087 | 207.939 | −24.720 | 1.00 | 50.00 | AAAB | O |
| ATOM | 305 | CB | ILE | A | 59 | 87.145 | 210.363 | −23.192 | 1.00 | 50.00 | AAAB | C |
| ATOM | 306 | CG1 | ILE | A | 59 | 87.206 | 209.109 | −22.339 | 1.00 | 50.00 | AAAB | C |
| ATOM | 307 | CG2 | ILE | A | 59 | 87.038 | 211.575 | −22.259 | 1.00 | 50.00 | AAAB | C |
| ATOM | 308 | CD1 | ILE | A | 59 | 88.590 | 208.830 | −21.797 | 1.00 | 50.00 | AAAB | C |
| ATOM | 309 | H | ILE | A | 59 | 84.570 | 210.127 | −22.582 | 0.00 | 0.00 | AAAB | H |
| ATOM | 310 | N | GLU | A | 60 | 86.091 | 209.423 | −26.409 | 1.00 | 50.00 | AAAB | N |
| ATOM | 311 | CA | GLU | A | 60 | 86.564 | 208.414 | −27.348 | 1.00 | 50.00 | AAAB | C |
| ATOM | 312 | C | GLU | A | 60 | 88.011 | 208.178 | −27.130 | 1.00 | 50.00 | AAAB | C |
| ATOM | 313 | O | GLU | A | 60 | 88.844 | 209.067 | −27.230 | 1.00 | 50.00 | AAAB | O |
| ATOM | 314 | CB | GLU | A | 60 | 86.466 | 208.838 | −28.796 | 1.00 | 50.00 | AAAB | C |
| ATOM | 315 | CG | GLU | A | 60 | 85.841 | 207.766 | −29.679 | 1.00 | 50.00 | AAAB | C |
| ATOM | 316 | CD | GLU | A | 60 | 85.086 | 208.442 | −30.798 | 1.00 | 50.00 | AAAB | C |
| ATOM | 317 | OE1 | GLU | A | 60 | 85.521 | 209.486 | −31.270 | 1.00 | 50.00 | AAAB | O |
| ATOM | 318 | OE2 | GLU | A | 60 | 84.039 | 207.937 | −31.168 | 1.00 | 50.00 | AAAB | O |
| ATOM | 319 | H | GLU | A | 60 | 85.865 | 210.362 | −26.653 | 0.00 | 0.00 | AAAB | H |
| ATOM | 320 | N | LEU | A | 61 | 88.259 | 206.913 | −26.856 | 1.00 | 50.00 | AAAB | N |
| ATOM | 321 | CA | LEU | A | 61 | 89.653 | 206.527 | −26.917 | 1.00 | 50.00 | AAAB | C |
| ATOM | 322 | C | LEU | A | 61 | 90.121 | 206.179 | −28.312 | 1.00 | 50.00 | AAAB | C |
| ATOM | 323 | O | LEU | A | 61 | 91.308 | 206.229 | −28.606 | 1.00 | 50.00 | AAAB | O |
| ATOM | 324 | CB | LEU | A | 61 | 89.898 | 205.407 | −25.930 | 1.00 | 50.00 | AAAB | C |
| ATOM | 325 | CG | LEU | A | 61 | 89.525 | 205.913 | −24.549 | 1.00 | 50.00 | AAAB | C |
| ATOM | 326 | CD1 | LEU | A | 61 | 89.273 | 204.743 | −23.625 | 1.00 | 50.00 | AAAB | C |
| ATOM | 327 | CD2 | LEU | A | 61 | 90.567 | 206.894 | −24.014 | 1.00 | 50.00 | AAAB | C |
| ATOM | 328 | H | LEU | A | 61 | 87.525 | 206.289 | −26.591 | 0.00 | 0.00 | AAAB | H |
| ATOM | 329 | N | SER | A | 62 | 89.134 | 205.841 | −29.171 | 1.00 | 99.99 | AAAB | N |
| ATOM | 330 | CA | SER | A | 62 | 89.476 | 205.390 | −30.521 | 1.00 | 99.99 | AAAB | C |
| ATOM | 331 | C | SER | A | 62 | 88.316 | 205.388 | −31.508 | 1.00 | 99.99 | AAAB | C |
| ATOM | 332 | O | SER | A | 62 | 87.145 | 205.364 | −31.151 | 1.00 | 99.99 | AAAB | O |
| ATOM | 333 | CB | SER | A | 62 | 90.113 | 203.998 | −30.446 | 1.00 | 99.99 | AAAB | C |
| ATOM | 334 | OG | SER | A | 62 | 90.598 | 203.566 | −31.720 | 1.00 | 99.99 | AAAB | O |
| ATOM | 335 | H | SER | A | 62 | 88.179 | 205.803 | −28.867 | 0.00 | 0.00 | AAAB | H |
| ATOM | 336 | HG | SER | A | 62 | 90.917 | 202.685 | −31.577 | 0.00 | 0.00 | AAAB | H |
| ATOM | 337 | N | ASN | A | 63 | 88.731 | 205.400 | −32.789 | 1.00 | 99.99 | AAAB | N |
| ATOM | 338 | CA | ASN | A | 63 | 87.764 | 205.325 | −33.884 | 1.00 | 99.99 | AAAB | C |
| ATOM | 339 | C | ASN | A | 63 | 87.861 | 204.030 | −34.689 | 1.00 | 99.99 | AAAB | C |
| ATOM | 340 | O | ASN | A | 63 | 88.886 | 203.357 | −34.693 | 1.00 | 99.99 | AAAB | O |
| ATOM | 341 | CB | ASN | A | 63 | 87.903 | 206.563 | −34.790 | 1.00 | 99.99 | AAAB | C |
| ATOM | 342 | CG | ASN | A | 63 | 89.313 | 206.711 | −35.359 | 1.00 | 99.99 | AAAB | C |
| ATOM | 343 | ND2 | ASN | A | 63 | 89.698 | 207.988 | −35.513 | 1.00 | 99.99 | AAAB | N |
| ATOM | 344 | OD1 | ASN | A | 63 | 90.020 | 205.748 | −35.639 | 1.00 | 99.99 | AAAB | O |
| ATOM | 345 | H | ASN | A | 63 | 89.710 | 205.371 | −32.990 | 0.00 | 0.00 | AAAB | H |
| ATOM | 346 | 1HD2 | ASN | A | 63 | 90.596 | 208.188 | −35.897 | 0.00 | 0.00 | AAAB | H |
| ATOM | 347 | 2HD2 | ASN | A | 63 | 89.111 | 208.753 | −35.252 | 0.00 | 0.00 | AAAB | H |
| ATOM | 348 | N | ILE | A | 64 | 86.754 | 203.721 | −35.393 | 1.00 | 99.99 | AAAB | N |
| ATOM | 349 | CA | ILE | A | 64 | 86.857 | 202.592 | −36.320 | 1.00 | 99.99 | AAAB | C |
| ATOM | 350 | C | ILE | A | 64 | 86.800 | 203.030 | −37.774 | 1.00 | 99.99 | AAAB | C |
| ATOM | 351 | O | ILE | A | 64 | 85.948 | 203.807 | −38.190 | 1.00 | 99.99 | AAAB | O |
| ATOM | 352 | CB | ILE | A | 64 | 85.841 | 201.457 | −36.037 | 1.00 | 99.99 | AAAB | C |
| ATOM | 353 | CG1 | ILE | A | 64 | 84.398 | 201.765 | −36.477 | 1.00 | 99.99 | AAAB | C |
| ATOM | 354 | CG2 | ILE | A | 64 | 85.919 | 201.062 | −34.555 | 1.00 | 99.99 | AAAB | C |
| ATOM | 355 | CD1 | ILE | A | 64 | 83.335 | 200.759 | −36.025 | 1.00 | 99.99 | AAAB | C |
| ATOM | 356 | H | ILE | A | 64 | 85.924 | 204.275 | −35.336 | 0.00 | 0.00 | AAAB | H |
| ATOM | 357 | N | LYS | A | 65 | 87.770 | 202.484 | −38.525 | 1.00 | 99.99 | AAAB | N |
| ATOM | 358 | CA | LYS | A | 65 | 87.810 | 202.778 | −39.953 | 1.00 | 99.99 | AAAB | C |
| ATOM | 359 | C | LYS | A | 65 | 88.277 | 201.587 | −40.776 | 1.00 | 99.99 | AAAB | C |
| ATOM | 360 | O | LYS | A | 65 | 88.962 | 200.688 | −40.301 | 1.00 | 99.99 | AAAB | O |
| ATOM | 361 | CB | LYS | A | 65 | 88.689 | 204.008 | −40.217 | 1.00 | 99.99 | AAAB | C |
| ATOM | 362 | CG | LYS | A | 65 | 90.130 | 203.816 | −39.744 | 1.00 | 99.99 | AAAB | C |
| ATOM | 363 | CD | LYS | A | 65 | 91.009 | 205.041 | −39.973 | 1.00 | 99.99 | AAAB | C |
| ATOM | 364 | CE | LYS | A | 65 | 92.461 | 204.761 | −39.587 | 1.00 | 99.99 | AAAB | C |
| ATOM | 365 | NZ | LYS | A | 65 | 93.019 | 203.730 | −40.477 | 1.00 | 99.99 | AAAB | N |
| ATOM | 366 | H | LYS | A | 65 | 88.493 | 201.940 | −38.100 | 0.00 | 0.00 | AAAB | H |
| ATOM | 367 | 1HZ | LYS | A | 65 | 94.010 | 203.548 | −40.216 | 0.00 | 0.00 | AAAB | H |
| ATOM | 368 | 2HZ | LYS | A | 65 | 92.982 | 204.055 | −41.466 | 0.00 | 0.00 | AAAB | H |
| ATOM | 369 | 3HZ | LYS | A | 65 | 92.474 | 202.849 | −40.383 | 0.00 | 0.00 | AAAB | H |
| ATOM | 370 | N | LYS | A | 66 | 87.874 | 201.634 | −42.057 | 1.00 | 99.99 | AAAB | N |
| ATOM | 371 | CA | LYS | A | 66 | 88.229 | 200.539 | −42.963 | 1.00 | 99.99 | AAAB | C |
| ATOM | 372 | C | LYS | A | 66 | 89.648 | 200.617 | −43.513 | 1.00 | 99.99 | AAAB | C |
| ATOM | 373 | O | LYS | A | 66 | 90.293 | 201.660 | −43.478 | 1.00 | 99.99 | AAAB | O |
| ATOM | 374 | CB | LYS | A | 66 | 87.207 | 200.462 | −44.099 | 1.00 | 99.99 | AAAB | C |
| ATOM | 375 | CG | LYS | A | 66 | 87.144 | 201.747 | −44.928 | 1.00 | 99.99 | AAAB | C |
| ATOM | 376 | CD | LYS | A | 66 | 86.084 | 201.697 | −46.026 | 1.00 | 99.99 | AAAB | C |
| ATOM | 377 | CE | LYS | A | 66 | 86.027 | 202.989 | −46.843 | 1.00 | 99.99 | AAAB | C |
| ATOM | 378 | NZ | LYS | A | 66 | 87.289 | 203.180 | −47.572 | 1.00 | 99.99 | AAAB | N |
| ATOM | 379 | H | LYS | A | 66 | 87.319 | 202.410 | −42.355 | 0.00 | 0.00 | AAAB | H |
| ATOM | 380 | 1HZ | LYS | A | 66 | 87.240 | 204.063 | −48.119 | 0.00 | 0.00 | AAAB | H |
| ATOM | 381 | 2HZ | LYS | A | 66 | 87.442 | 202.377 | −48.216 | 0.00 | 0.00 | AAAB | H |
| ATOM | 382 | 3HZ | LYS | A | 66 | 88.077 | 203.234 | −46.896 | 0.00 | 0.00 | AAAB | H |
| ATOM | 383 | N | ASN | A | 67 | 90.091 | 199.447 | −44.020 | 1.00 | 99.99 | AAAB | N |
| ATOM | 384 | CA | ASN | A | 67 | 91.438 | 199.344 | −44.579 | 1.00 | 99.99 | AAAB | C |
| ATOM | 385 | C | ASN | A | 67 | 91.662 | 198.158 | −45.503 | 1.00 | 99.99 | AAAB | C |
| ATOM | 386 | O | ASN | A | 67 | 91.172 | 197.049 | −45.331 | 1.00 | 99.99 | AAAB | O |
| ATOM | 387 | CB | ASN | A | 67 | 92.535 | 199.399 | −43.491 | 1.00 | 99.99 | AAAB | C |
| ATOM | 388 | CG | ASN | A | 67 | 92.382 | 198.310 | −42.439 | 1.00 | 99.99 | AAAB | C |
| ATOM | 389 | ND2 | ASN | A | 67 | 92.762 | 198.705 | −41.211 | 1.00 | 99.99 | AAAB | N |
| ATOM | 390 | OD1 | ASN | A | 67 | 91.949 | 197.194 | −42.691 | 1.00 | 99.99 | AAAB | O |
| ATOM | 391 | H | ASN | A | 67 | 89.506 | 198.636 | −44.019 | 0.00 | 0.00 | AAAB | H |
| ATOM | 392 | 1HD2 | ASN | A | 67 | 92.686 | 198.051 | −40.459 | 0.00 | 0.00 | AAAB | H |
| ATOM | 393 | 2HD2 | ASN | A | 67 | 93.090 | 199.629 | −41.029 | 0.00 | 0.00 | AAAB | H |
| ATOM | 394 | N | LYS | A | 68 | 92.499 | 198.487 | −46.491 | 1.00 | 99.99 | AAAB | N |
| ATOM | 395 | CA | LYS | A | 68 | 93.239 | 197.479 | −47.243 | 1.00 | 99.99 | AAAB | C |
| ATOM | 396 | C | LYS | A | 68 | 94.680 | 197.932 | −47.101 | 1.00 | 99.99 | AAAB | C |
| ATOM | 397 | O | LYS | A | 68 | 94.907 | 199.114 | −46.856 | 1.00 | 99.99 | AAAB | O |
| ATOM | 398 | CB | LYS | A | 68 | 92.771 | 197.460 | −48.694 | 1.00 | 99.99 | AAAB | C |
| ATOM | 399 | CG | LYS | A | 68 | 92.762 | 198.885 | −49.221 | 1.00 | 99.99 | AAAB | C |
| ATOM | 400 | CD | LYS | A | 68 | 92.447 | 199.038 | −50.694 | 1.00 | 99.99 | AAAB | C |
| ATOM | 401 | CE | LYS | A | 68 | 93.073 | 200.349 | −51.129 | 1.00 | 99.99 | AAAB | C |
| ATOM | 402 | NZ | LYS | A | 68 | 94.517 | 200.218 | −50.902 | 1.00 | 99.99 | AAAB | N |
| ATOM | 403 | H | LYS | A | 68 | 92.758 | 199.446 | −46.605 | 0.00 | 0.00 | AAAB | H |
| ATOM | 404 | 1HZ | LYS | A | 68 | 94.963 | 201.128 | −51.128 | 0.00 | 0.00 | AAAB | H |
| ATOM | 405 | 2HZ | LYS | A | 68 | 94.919 | 199.491 | −51.528 | 0.00 | 0.00 | AAAB | H |
| ATOM | 406 | 3HZ | LYS | A | 68 | 94.757 | 199.980 | −49.918 | 0.00 | 0.00 | AAAB | H |
| ATOM | 407 | N | CYS | A | 69 | 95.605 | 196.941 | −47.131 | 1.00 | 50.00 | AAAB | N |
| ATOM | 408 | CA | CYS | A | 69 | 96.701 | 196.971 | −46.151 | 1.00 | 50.00 | AAAB | C |
| ATOM | 409 | C | CYS | A | 69 | 96.158 | 196.809 | −44.733 | 1.00 | 50.00 | AAAB | C |
| ATOM | 410 | O | CYS | A | 69 | 94.967 | 196.947 | −44.481 | 1.00 | 50.00 | AAAB | O |
| ATOM | 411 | CB | CYS | A | 69 | 97.590 | 198.225 | −46.304 | 1.00 | 50.00 | AAAB | C |
| ATOM | 412 | SG | CYS | A | 69 | 98.217 | 198.930 | −44.765 | 1.00 | 50.00 | AAAB | S |
| ATOM | 413 | H | CYS | A | 69 | 95.395 | 196.092 | −47.612 | 0.00 | 0.00 | AAAB | H |
| ATOM | 414 | N | ASN | A | 70 | 97.079 | 196.510 | −43.806 | 1.00 | 50.00 | AAAB | N |
| ATOM | 415 | CA | ASN | A | 70 | 96.585 | 196.424 | −42.437 | 1.00 | 50.00 | AAAB | C |
| ATOM | 416 | C | ASN | A | 70 | 97.266 | 197.428 | −41.523 | 1.00 | 50.00 | AAAB | C |
| ATOM | 417 | O | ASN | A | 70 | 98.459 | 197.355 | −41.254 | 1.00 | 50.00 | AAAB | O |
| ATOM | 418 | CB | ASN | A | 70 | 96.702 | 194.980 | −41.921 | 1.00 | 50.00 | AAAB | C |
| ATOM | 419 | CG | ASN | A | 70 | 96.043 | 194.806 | −40.560 | 1.00 | 50.00 | AAAB | C |
| ATOM | 420 | ND2 | ASN | A | 70 | 95.410 | 193.634 | −40.409 | 1.00 | 50.00 | AAAB | N |
| ATOM | 421 | OD1 | ASN | A | 70 | 96.114 | 195.655 | −39.684 | 1.00 | 50.00 | AAAB | O |
| ATOM | 422 | H | ASN | A | 70 | 98.043 | 196.365 | −44.029 | 0.00 | 0.00 | AAAB | H |
| ATOM | 423 | 1HD2 | ASN | A | 70 | 95.006 | 193.428 | −39.517 | 0.00 | 0.00 | AAAB | H |
| ATOM | 424 | 2HD2 | ASN | A | 70 | 95.333 | 192.951 | −41.135 | 0.00 | 0.00 | AAAB | H |
| ATOM | 425 | N | GLY | A | 71 | 96.411 | 198.350 | −41.033 | 1.00 | 50.00 | AAAB | N |
| ATOM | 426 | CA | GLY | A | 71 | 96.807 | 199.186 | −39.901 | 1.00 | 50.00 | AAAB | C |
| ATOM | 427 | C | GLY | A | 71 | 96.868 | 198.452 | −38.566 | 1.00 | 50.00 | AAAB | C |
| ATOM | 428 | O | GLY | A | 71 | 95.939 | 198.483 | −37.771 | 1.00 | 50.00 | AAAB | O |
| ATOM | 429 | H | GLY | A | 71 | 95.474 | 198.392 | −41.382 | 0.00 | 0.00 | AAAB | H |
| ATOM | 430 | N | THR | A | 72 | 98.036 | 197.807 | −38.360 | 1.00 | 50.00 | AAAB | N |
| ATOM | 431 | CA | THR | A | 72 | 98.304 | 197.010 | −37.155 | 1.00 | 50.00 | AAAB | C |
| ATOM | 432 | C | THR | A | 72 | 98.368 | 197.796 | −35.856 | 1.00 | 50.00 | AAAB | C |
| ATOM | 433 | O | THR | A | 72 | 97.976 | 197.350 | −34.786 | 1.00 | 50.00 | AAAB | O |
| ATOM | 434 | CB | THR | A | 72 | 99.607 | 196.224 | −37.335 | 1.00 | 50.00 | AAAB | C |
| ATOM | 435 | CG2 | THR | A | 72 | 99.546 | 195.287 | −38.542 | 1.00 | 50.00 | AAAB | C |
| ATOM | 436 | OG1 | THR | A | 72 | 100.715 | 197.122 | −37.480 | 1.00 | 50.00 | AAAB | O |
| ATOM | 437 | H | THR | A | 72 | 98.732 | 197.831 | −39.078 | 0.00 | 0.00 | AAAB | H |
| ATOM | 438 | HG1 | THR | A | 72 | 101.492 | 196.574 | −37.508 | 0.00 | 0.00 | AAAB | H |
| ATOM | 439 | N | ASP | A | 73 | 98.877 | 199.025 | −36.005 | 1.00 | 50.00 | AAAB | N |
| ATOM | 440 | CA | ASP | A | 73 | 99.042 | 199.833 | −34.802 | 1.00 | 50.00 | AAAB | C |
| ATOM | 441 | C | ASP | A | 73 | 97.773 | 200.246 | −34.102 | 1.00 | 50.00 | AAAB | C |
| ATOM | 442 | O | ASP | A | 73 | 97.742 | 200.458 | −32.897 | 1.00 | 50.00 | AAAB | O |
| ATOM | 443 | CB | ASP | A | 73 | 99.832 | 201.062 | −35.147 | 1.00 | 50.00 | AAAB | C |
| ATOM | 444 | CG | ASP | A | 73 | 101.225 | 200.634 | −35.538 | 1.00 | 50.00 | AAAB | C |
| ATOM | 445 | OD1 | ASP | A | 73 | 101.819 | 199.787 | −34.868 | 1.00 | 50.00 | AAAB | O |
| ATOM | 446 | OD2 | ASP | A | 73 | 101.735 | 201.192 | −36.496 | 1.00 | 50.00 | AAAB | O |
| ATOM | 447 | H | ASP | A | 73 | 99.331 | 199.295 | −36.853 | 0.00 | 0.00 | AAAB | H |
| ATOM | 448 | N | ALA | A | 74 | 96.721 | 200.317 | −34.934 | 1.00 | 50.00 | AAAB | N |
| ATOM | 449 | CA | ALA | A | 74 | 95.376 | 200.469 | −34.389 | 1.00 | 50.00 | AAAB | C |
| ATOM | 450 | C | ALA | A | 74 | 94.931 | 199.287 | −33.539 | 1.00 | 50.00 | AAAB | C |
| ATOM | 451 | O | ALA | A | 74 | 94.314 | 199.450 | −32.498 | 1.00 | 50.00 | AAAB | O |
| ATOM | 452 | CB | ALA | A | 74 | 94.378 | 200.670 | −35.530 | 1.00 | 50.00 | AAAB | C |
| ATOM | 453 | H | ALA | A | 74 | 96.856 | 200.157 | −35.910 | 0.00 | 0.00 | AAAB | H |
| ATOM | 454 | N | LYS | A | 75 | 95.301 | 198.090 | −34.036 | 1.00 | 99.99 | AAAB | N |
| ATOM | 455 | CA | LYS | A | 75 | 95.043 | 196.880 | −33.254 | 1.00 | 99.99 | AAAB | C |
| ATOM | 456 | C | LYS | A | 75 | 96.144 | 196.560 | −32.247 | 1.00 | 99.99 | AAAB | C |
| ATOM | 457 | O | LYS | A | 75 | 96.995 | 195.692 | −32.417 | 1.00 | 99.99 | AAAB | O |
| ATOM | 458 | CB | LYS | A | 75 | 94.696 | 195.683 | −34.164 | 1.00 | 99.99 | AAAB | C |
| ATOM | 459 | CG | LYS | A | 75 | 95.764 | 195.294 | −35.191 | 1.00 | 99.99 | AAAB | C |
| ATOM | 460 | CD | LYS | A | 75 | 95.507 | 193.993 | −35.948 | 1.00 | 99.99 | AAAB | C |
| ATOM | 461 | CE | LYS | A | 75 | 96.700 | 193.577 | −36.817 | 1.00 | 99.99 | AAAB | C |
| ATOM | 462 | NZ | LYS | A | 75 | 97.880 | 193.299 | −35.979 | 1.00 | 99.99 | AAAB | N |
| ATOM | 463 | H | LYS | A | 75 | 95.825 | 198.045 | −34.885 | 0.00 | 0.00 | AAAB | H |
| ATOM | 464 | 1HZ | LYS | A | 75 | 98.694 | 193.081 | −36.589 | 0.00 | 0.00 | AAAB | H |
| ATOM | 465 | 2HZ | LYS | A | 75 | 97.683 | 192.483 | −35.365 | 0.00 | 0.00 | AAAB | H |
| ATOM | 466 | 3HZ | LYS | A | 75 | 98.116 | 194.126 | −35.393 | 0.00 | 0.00 | AAAB | H |
| ATOM | 467 | N | VAL | A | 76 | 96.065 | 197.330 | −31.151 | 1.00 | 99.99 | AAAB | N |
| ATOM | 468 | CA | VAL | A | 76 | 97.013 | 197.081 | −30.073 | 1.00 | 99.99 | AAAB | C |
| ATOM | 469 | C | VAL | A | 76 | 96.311 | 196.512 | −28.843 | 1.00 | 99.99 | AAAB | C |
| ATOM | 470 | O | VAL | A | 76 | 95.158 | 196.810 | −28.558 | 1.00 | 99.99 | AAAB | O |
| ATOM | 471 | CB | VAL | A | 76 | 97.817 | 198.371 | −29.791 | 1.00 | 99.99 | AAAB | C |
| ATOM | 472 | CG1 | VAL | A | 76 | 96.930 | 199.509 | −29.277 | 1.00 | 99.99 | AAAB | C |
| ATOM | 473 | CG2 | VAL | A | 76 | 99.049 | 198.135 | −28.910 | 1.00 | 99.99 | AAAB | C |
| ATOM | 474 | H | VAL | A | 76 | 95.382 | 198.059 | −31.104 | 0.00 | 0.00 | AAAB | H |
| ATOM | 475 | N | LYS | A | 77 | 97.065 | 195.657 | −28.133 | 1.00 | 99.99 | AAAB | N |
| ATOM | 476 | CA | LYS | A | 77 | 96.551 | 194.947 | −26.958 | 1.00 | 99.99 | AAAB | C |
| ATOM | 477 | C | LYS | A | 77 | 96.270 | 195.785 | −25.707 | 1.00 | 99.99 | AAAB | C |
| ATOM | 478 | O | LYS | A | 77 | 96.050 | 195.247 | −24.632 | 1.00 | 99.99 | AAAB | O |
| ATOM | 479 | CB | LYS | A | 77 | 97.537 | 193.829 | −26.594 | 1.00 | 99.99 | AAAB | C |
| ATOM | 480 | CG | LYS | A | 77 | 98.913 | 194.384 | −26.196 | 1.00 | 99.99 | AAAB | C |
| ATOM | 481 | CD | LYS | A | 77 | 99.894 | 193.335 | −25.671 | 1.00 | 99.99 | AAAB | C |
| ATOM | 482 | CE | LYS | A | 77 | 101.236 | 193.947 | −25.252 | 1.00 | 99.99 | AAAB | C |
| ATOM | 483 | NZ | LYS | A | 77 | 101.052 | 194.879 | −24.125 | 1.00 | 99.99 | AAAB | N |
| ATOM | 484 | H | LYS | A | 77 | 97.995 | 195.493 | −28.462 | 0.00 | 0.00 | AAAB | H |
| ATOM | 485 | 1HZ | LYS | A | 77 | 101.974 | 195.301 | −23.887 | 0.00 | 0.00 | AAAB | H |
| ATOM | 486 | 2HZ | LYS | A | 77 | 100.684 | 194.363 | −23.301 | 0.00 | 0.00 | AAAB | H |
| ATOM | 487 | 3HZ | LYS | A | 77 | 100.385 | 195.630 | −24.398 | 0.00 | 0.00 | AAAB | H |
| ATOM | 488 | N | LEU | A | 78 | 96.369 | 197.119 | −25.861 | 1.00 | 50.00 | AAAB | N |
| ATOM | 489 | CA | LEU | A | 78 | 96.826 | 197.857 | −24.689 | 1.00 | 50.00 | AAAB | C |
| ATOM | 490 | C | LEU | A | 78 | 95.816 | 198.702 | −23.940 | 1.00 | 50.00 | AAAB | C |
| ATOM | 491 | O | LEU | A | 78 | 95.742 | 198.637 | −22.722 | 1.00 | 50.00 | AAAB | O |
| ATOM | 492 | CB | LEU | A | 78 | 98.083 | 198.655 | −25.039 | 1.00 | 50.00 | AAAB | C |
| ATOM | 493 | CG | LEU | A | 78 | 99.059 | 198.837 | −23.869 | 1.00 | 50.00 | AAAB | C |
| ATOM | 494 | CD1 | LEU | A | 78 | 100.485 | 199.014 | −24.385 | 1.00 | 50.00 | AAAB | C |
| ATOM | 495 | CD2 | LEU | A | 78 | 98.672 | 199.949 | −22.892 | 1.00 | 50.00 | AAAB | C |
| ATOM | 496 | H | LEU | A | 78 | 96.222 | 197.556 | −26.747 | 0.00 | 0.00 | AAAB | H |
| ATOM | 497 | N | ILE | A | 79 | 95.055 | 199.512 | −24.686 | 1.00 | 50.00 | AAAB | N |
| ATOM | 498 | CA | ILE | A | 79 | 94.184 | 200.422 | −23.931 | 1.00 | 50.00 | AAAB | C |
| ATOM | 499 | C | ILE | A | 79 | 93.048 | 199.723 | −23.194 | 1.00 | 50.00 | AAAB | C |
| ATOM | 500 | O | ILE | A | 79 | 92.656 | 200.064 | −22.085 | 1.00 | 50.00 | AAAB | O |
| ATOM | 501 | CB | ILE | A | 79 | 93.689 | 201.580 | −24.812 | 1.00 | 50.00 | AAAB | C |
| ATOM | 502 | CG1 | ILE | A | 79 | 92.946 | 201.120 | −26.070 | 1.00 | 50.00 | AAAB | C |
| ATOM | 503 | CG2 | ILE | A | 79 | 94.888 | 202.451 | −25.195 | 1.00 | 50.00 | AAAB | C |
| ATOM | 504 | CD1 | ILE | A | 79 | 92.480 | 202.298 | −26.931 | 1.00 | 50.00 | AAAB | C |
| ATOM | 505 | H | ILE | A | 79 | 95.113 | 199.519 | −25.684 | 0.00 | 0.00 | AAAB | H |
| ATOM | 506 | N | LYS | A | 80 | 92.605 | 198.653 | −23.872 | 1.00 | 50.00 | AAAB | N |
| ATOM | 507 | CA | LYS | A | 80 | 91.584 | 197.764 | −23.340 | 1.00 | 50.00 | AAAB | C |
| ATOM | 508 | C | LYS | A | 80 | 91.970 | 197.101 | −22.016 | 1.00 | 50.00 | AAAB | C |
| ATOM | 509 | O | LYS | A | 80 | 91.227 | 197.120 | −21.039 | 1.00 | 50.00 | AAAB | O |
| ATOM | 510 | CB | LYS | A | 80 | 91.316 | 196.753 | −24.450 | 1.00 | 50.00 | AAAB | C |
| ATOM | 511 | CG | LYS | A | 80 | 90.300 | 195.688 | −24.085 | 1.00 | 50.00 | AAAB | C |
| ATOM | 512 | CD | LYS | A | 80 | 88.911 | 196.255 | −23.885 | 1.00 | 50.00 | AAAB | C |
| ATOM | 513 | CE | LYS | A | 80 | 88.044 | 195.146 | −23.317 | 1.00 | 50.00 | AAAB | C |
| ATOM | 514 | NZ | LYS | A | 80 | 87.982 | 195.280 | −21.854 | 1.00 | 50.00 | AAAB | N |
| ATOM | 515 | H | LYS | A | 80 | 92.992 | 198.462 | −24.773 | 0.00 | 0.00 | AAAB | H |
| ATOM | 516 | 1HZ | LYS | A | 80 | 87.058 | 194.936 | −21.523 | 0.00 | 0.00 | AAAB | H |
| ATOM | 517 | 2HZ | LYS | A | 80 | 88.079 | 196.279 | −21.577 | 0.00 | 0.00 | AAAB | H |
| ATOM | 518 | 3HZ | LYS | A | 80 | 88.738 | 194.725 | −21.411 | 0.00 | 0.00 | AAAB | H |
| ATOM | 519 | N | GLN | A | 81 | 93.195 | 196.522 | −22.045 | 1.00 | 50.00 | AAAB | N |
| ATOM | 520 | CA | GLN | A | 81 | 93.743 | 195.836 | −20.871 | 1.00 | 50.00 | AAAB | C |
| ATOM | 521 | C | GLN | A | 81 | 93.959 | 196.748 | −19.680 | 1.00 | 50.00 | AAAB | C |
| ATOM | 522 | O | GLN | A | 81 | 93.704 | 196.407 | −18.533 | 1.00 | 50.00 | AAAB | O |
| ATOM | 523 | CB | GLN | A | 81 | 95.033 | 195.057 | −21.200 | 1.00 | 50.00 | AAAB | C |
| ATOM | 524 | CG | GLN | A | 81 | 96.221 | 195.975 | −21.509 | 1.00 | 50.00 | AAAB | C |
| ATOM | 525 | CD | GLN | A | 81 | 97.523 | 195.269 | −21.778 | 1.00 | 50.00 | AAAB | C |
| ATOM | 526 | NE2 | GLN | A | 81 | 98.544 | 195.817 | −21.100 | 1.00 | 50.00 | AAAB | N |
| ATOM | 527 | OE1 | GLN | A | 81 | 97.628 | 194.332 | −22.555 | 1.00 | 50.00 | AAAB | O |
| ATOM | 528 | H | GLN | A | 81 | 93.738 | 196.601 | −22.881 | 0.00 | 0.00 | AAAB | H |
| ATOM | 529 | 1HE2 | GLN | A | 81 | 99.468 | 195.473 | −21.249 | 0.00 | 0.00 | AAAB | H |
| ATOM | 530 | 2HE2 | GLN | A | 81 | 98.385 | 196.568 | −20.457 | 0.00 | 0.00 | AAAB | H |
| ATOM | 531 | N | GLU | A | 82 | 94.420 | 197.958 | −20.039 | 1.00 | 50.00 | AAAB | N |
| ATOM | 532 | CA | GLU | A | 82 | 94.664 | 198.980 | −19.043 | 1.00 | 50.00 | AAAB | C |
| ATOM | 533 | C | GLU | A | 82 | 93.362 | 199.334 | −18.337 | 1.00 | 50.00 | AAAB | C |
| ATOM | 534 | O | GLU | A | 82 | 93.305 | 199.492 | −17.126 | 1.00 | 50.00 | AAAB | O |
| ATOM | 535 | CB | GLU | A | 82 | 95.369 | 200.121 | −19.794 | 1.00 | 50.00 | AAAB | C |
| ATOM | 536 | CG | GLU | A | 82 | 95.657 | 201.425 | −19.055 | 1.00 | 50.00 | AAAB | C |
| ATOM | 537 | CD | GLU | A | 82 | 94.353 | 202.165 | −18.859 | 1.00 | 50.00 | AAAB | C |
| ATOM | 538 | OE1 | GLU | A | 82 | 93.909 | 202.837 | −19.788 | 1.00 | 50.00 | AAAB | O |
| ATOM | 539 | OE2 | GLU | A | 82 | 93.785 | 202.063 | −17.774 | 1.00 | 50.00 | AAAB | O |
| ATOM | 540 | H | GLU | A | 82 | 94.604 | 198.162 | −21.000 | 0.00 | 0.00 | AAAB | H |
| ATOM | 541 | N | LEU | A | 83 | 92.307 | 199.414 | −19.168 | 1.00 | 50.00 | AAAB | N |
| ATOM | 542 | CA | LEU | A | 83 | 90.999 | 199.752 | −18.624 | 1.00 | 50.00 | AAAB | C |
| ATOM | 543 | C | LEU | A | 83 | 90.198 | 198.552 | −18.175 | 1.00 | 50.00 | AAAB | C |
| ATOM | 544 | O | LEU | A | 83 | 88.986 | 198.622 | −18.156 | 1.00 | 50.00 | AAAB | O |
| ATOM | 545 | CB | LEU | A | 83 | 90.158 | 200.472 | −19.665 | 1.00 | 50.00 | AAAB | C |
| ATOM | 546 | CG | LEU | A | 83 | 90.672 | 201.843 | −20.059 | 1.00 | 50.00 | AAAB | C |
| ATOM | 547 | CD1 | LEU | A | 83 | 89.981 | 202.274 | −21.340 | 1.00 | 50.00 | AAAB | C |
| ATOM | 548 | CD2 | LEU | A | 83 | 90.554 | 202.874 | −18.932 | 1.00 | 50.00 | AAAB | C |
| ATOM | 549 | H | LEU | A | 83 | 92.392 | 199.113 | −20.120 | 0.00 | 0.00 | AAAB | H |
| ATOM | 550 | N | ASP | A | 84 | 90.890 | 197.445 | −17.845 | 1.00 | 50.00 | AAAB | N |
| ATOM | 551 | CA | ASP | A | 84 | 90.140 | 196.266 | −17.397 | 1.00 | 50.00 | AAAB | C |
| ATOM | 552 | C | ASP | A | 84 | 89.796 | 196.261 | −15.933 | 1.00 | 50.00 | AAAB | C |
| ATOM | 553 | O | ASP | A | 84 | 88.731 | 195.825 | −15.527 | 1.00 | 50.00 | AAAB | O |
| ATOM | 554 | CB | ASP | A | 84 | 90.881 | 194.962 | −17.613 | 1.00 | 50.00 | AAAB | C |
| ATOM | 555 | CG | ASP | A | 84 | 90.873 | 194.552 | −19.060 | 1.00 | 50.00 | AAAB | C |
| ATOM | 556 | OD1 | ASP | A | 84 | 89.955 | 194.909 | −19.794 | 1.00 | 50.00 | AAAB | O |
| ATOM | 557 | OD2 | ASP | A | 84 | 91.795 | 193.842 | −19.447 | 1.00 | 50.00 | AAAB | O |
| ATOM | 558 | H | ASP | A | 84 | 91.875 | 197.398 | −18.006 | 0.00 | 0.00 | AAAB | H |
| ATOM | 559 | N | LYS | A | 85 | 90.753 | 196.785 | −15.165 | 1.00 | 50.00 | AAAB | N |
| ATOM | 560 | CA | LYS | A | 85 | 90.511 | 196.984 | −13.740 | 1.00 | 50.00 | AAAB | C |
| ATOM | 561 | C | LYS | A | 85 | 89.584 | 198.147 | −13.510 | 1.00 | 50.00 | AAAB | C |
| ATOM | 562 | O | LYS | A | 85 | 88.761 | 198.170 | −12.609 | 1.00 | 50.00 | AAAB | O |
| ATOM | 563 | CB | LYS | A | 85 | 91.835 | 197.284 | −13.081 | 1.00 | 50.00 | AAAB | C |
| ATOM | 564 | CG | LYS | A | 85 | 92.779 | 196.084 | −13.100 | 1.00 | 50.00 | AAAB | C |
| ATOM | 565 | CD | LYS | A | 85 | 92.289 | 194.942 | −12.206 | 1.00 | 50.00 | AAAB | C |
| ATOM | 566 | CE | LYS | A | 85 | 91.849 | 195.440 | −10.825 | 1.00 | 50.00 | AAAB | C |
| ATOM | 567 | NZ | LYS | A | 85 | 91.981 | 194.376 | −9.830 | 1.00 | 50.00 | AAAB | N |
| ATOM | 568 | H | LYS | A | 85 | 91.590 | 197.148 | −15.572 | 0.00 | 0.00 | AAAB | H |
| ATOM | 569 | 1HZ | LYS | A | 85 | 92.010 | 194.790 | −8.877 | 0.00 | 0.00 | AAAB | H |
| ATOM | 570 | 2HZ | LYS | A | 85 | 92.862 | 193.859 | −10.019 | 0.00 | 0.00 | AAAB | H |
| ATOM | 571 | 3HZ | LYS | A | 85 | 91.167 | 193.738 | −9.924 | 0.00 | 0.00 | AAAB | H |
| ATOM | 572 | N | TYR | A | 86 | 89.789 | 199.080 | −14.461 | 1.00 | 50.00 | AAAB | N |
| ATOM | 573 | CA | TYR | A | 86 | 88.824 | 200.086 | −14.854 | 1.00 | 50.00 | AAAB | C |
| ATOM | 574 | C | TYR | A | 86 | 87.840 | 199.644 | −15.950 | 1.00 | 50.00 | AAAB | C |
| ATOM | 575 | O | TYR | A | 86 | 87.385 | 200.478 | −16.722 | 1.00 | 50.00 | AAAB | O |
| ATOM | 576 | CB | TYR | A | 86 | 89.482 | 201.375 | −15.355 | 1.00 | 50.00 | AAAB | C |
| ATOM | 577 | CG | TYR | A | 86 | 90.340 | 202.171 | −14.392 | 1.00 | 50.00 | AAAB | C |
| ATOM | 578 | CD1 | TYR | A | 86 | 91.304 | 201.559 | −13.561 | 1.00 | 50.00 | AAAB | C |
| ATOM | 579 | CD2 | TYR | A | 86 | 90.198 | 203.572 | −14.445 | 1.00 | 50.00 | AAAB | C |
| ATOM | 580 | CE1 | TYR | A | 86 | 92.250 | 202.367 | −12.905 | 1.00 | 50.00 | AAAB | C |
| ATOM | 581 | CE2 | TYR | A | 86 | 91.144 | 204.378 | −13.797 | 1.00 | 50.00 | AAAB | C |
| ATOM | 582 | CZ | TYR | A | 86 | 92.198 | 203.764 | −13.089 | 1.00 | 50.00 | AAAB | C |
| ATOM | 583 | OH | TYR | A | 86 | 93.214 | 204.555 | −12.587 | 1.00 | 50.00 | AAAB | O |
| ATOM | 584 | H | TYR | A | 86 | 90.623 | 199.022 | −15.004 | 0.00 | 0.00 | AAAB | H |
| ATOM | 585 | HH | TYR | A | 86 | 93.142 | 205.435 | −12.938 | 0.00 | 0.00 | AAAB | H |
| ATOM | 586 | N | LYS | A | 87 | 87.483 | 198.330 | −15.955 | 1.00 | 50.00 | AAAB | N |
| ATOM | 587 | CA | LYS | A | 87 | 86.188 | 197.825 | −16.469 | 1.00 | 50.00 | AAAB | C |
| ATOM | 588 | C | LYS | A | 87 | 85.407 | 196.885 | −15.522 | 1.00 | 50.00 | AAAB | C |
| ATOM | 589 | O | LYS | A | 87 | 84.198 | 196.715 | −15.623 | 1.00 | 50.00 | AAAB | O |
| ATOM | 590 | CB | LYS | A | 87 | 86.250 | 197.275 | −17.890 | 1.00 | 50.00 | AAAB | C |
| ATOM | 591 | CG | LYS | A | 87 | 84.863 | 197.076 | −18.516 | 1.00 | 50.00 | AAAB | C |
| ATOM | 592 | CD | LYS | A | 87 | 84.000 | 198.341 | −18.623 | 1.00 | 50.00 | AAAB | C |
| ATOM | 593 | CE | LYS | A | 87 | 82.692 | 198.058 | −19.363 | 1.00 | 50.00 | AAAB | C |
| ATOM | 594 | NZ | LYS | A | 87 | 81.887 | 197.103 | −18.590 | 1.00 | 50.00 | AAAB | N |
| ATOM | 595 | H | LYS | A | 87 | 88.245 | 197.726 | −15.771 | 0.00 | 0.00 | AAAB | H |
| ATOM | 596 | 1HZ | LYS | A | 87 | 81.065 | 196.791 | −19.146 | 0.00 | 0.00 | AAAB | H |
| ATOM | 597 | 2HZ | LYS | A | 87 | 81.559 | 197.565 | −17.717 | 0.00 | 0.00 | AAAB | H |
| ATOM | 598 | 3HZ | LYS | A | 87 | 82.469 | 196.277 | −18.342 | 0.00 | 0.00 | AAAB | H |
| ATOM | 599 | N | ASN | A | 88 | 86.145 | 196.305 | −14.561 | 1.00 | 50.00 | AAAB | N |
| ATOM | 600 | CA | ASN | A | 88 | 85.516 | 195.311 | −13.678 | 1.00 | 50.00 | AAAB | C |
| ATOM | 601 | C | ASN | A | 88 | 84.835 | 195.822 | −12.400 | 1.00 | 50.00 | AAAB | C |
| ATOM | 602 | O | ASN | A | 88 | 83.652 | 195.603 | −12.198 | 1.00 | 50.00 | AAAB | O |
| ATOM | 603 | CB | ASN | A | 88 | 86.508 | 194.187 | −13.364 | 1.00 | 50.00 | AAAB | C |
| ATOM | 604 | CG | ASN | A | 88 | 87.002 | 193.546 | −14.649 | 1.00 | 50.00 | AAAB | C |
| ATOM | 605 | ND2 | ASN | A | 88 | 88.288 | 193.167 | −14.590 | 1.00 | 50.00 | AAAB | N |
| ATOM | 606 | OD1 | ASN | A | 88 | 86.286 | 193.411 | −15.634 | 1.00 | 50.00 | AAAB | O |
| ATOM | 607 | H | ASN | A | 88 | 87.121 | 196.507 | −14.502 | 0.00 | 0.00 | AAAB | H |
| ATOM | 608 | 1HD2 | ASN | A | 88 | 88.697 | 192.720 | −15.384 | 0.00 | 0.00 | AAAB | H |
| ATOM | 609 | 2HD2 | ASN | A | 88 | 88.853 | 193.320 | −13.780 | 0.00 | 0.00 | AAAB | H |
| ATOM | 610 | N | ALA | A | 89 | 85.613 | 196.540 | −11.552 | 1.00 | 50.00 | AAAB | N |
| ATOM | 611 | CA | ALA | A | 89 | 85.099 | 197.234 | −10.350 | 1.00 | 50.00 | AAAB | C |
| ATOM | 612 | C | ALA | A | 89 | 83.820 | 198.045 | −10.495 | 1.00 | 50.00 | AAAB | C |
| ATOM | 613 | O | ALA | A | 89 | 83.013 | 198.202 | −9.593 | 1.00 | 50.00 | AAAB | O |
| ATOM | 614 | CB | ALA | A | 89 | 86.151 | 198.177 | −9.755 | 1.00 | 50.00 | AAAB | C |
| ATOM | 615 | H | ALA | A | 89 | 86.588 | 196.616 | −11.754 | 0.00 | 0.00 | AAAB | H |
| ATOM | 616 | N | VAL | A | 90 | 83.653 | 198.541 | −11.706 | 1.00 | 50.00 | AAAB | N |
| ATOM | 617 | CA | VAL | A | 90 | 82.417 | 199.188 | −12.044 | 1.00 | 50.00 | AAAB | C |
| ATOM | 618 | C | VAL | A | 90 | 81.231 | 198.361 | −12.378 | 1.00 | 50.00 | AAAB | C |
| ATOM | 619 | O | VAL | A | 90 | 80.138 | 198.690 | −11.956 | 1.00 | 50.00 | AAAB | O |
| ATOM | 620 | CB | VAL | A | 90 | 82.814 | 200.066 | −13.143 | 1.00 | 50.00 | AAAB | C |
| ATOM | 621 | CG1 | VAL | A | 90 | 82.287 | 199.735 | −14.578 | 1.00 | 50.00 | AAAB | C |
| ATOM | 622 | CG2 | VAL | A | 90 | 83.036 | 201.382 | −12.404 | 1.00 | 50.00 | AAAB | C |
| ATOM | 623 | H | VAL | A | 90 | 84.424 | 198.604 | −12.333 | 0.00 | 0.00 | AAAB | H |
| ATOM | 624 | N | THR | A | 91 | 81.469 | 197.280 | −13.138 | 1.00 | 50.00 | AAAB | N |
| ATOM | 625 | CA | THR | A | 91 | 80.320 | 196.433 | −13.395 | 1.00 | 50.00 | AAAB | C |
| ATOM | 626 | C | THR | A | 91 | 79.837 | 195.900 | −12.065 | 1.00 | 50.00 | AAAB | C |
| ATOM | 627 | O | THR | A | 91 | 78.653 | 195.895 | −11.771 | 1.00 | 50.00 | AAAB | O |
| ATOM | 628 | CB | THR | A | 91 | 80.680 | 195.329 | −14.392 | 1.00 | 50.00 | AAAB | C |
| ATOM | 629 | CG2 | THR | A | 91 | 80.983 | 195.913 | −15.768 | 1.00 | 50.00 | AAAB | C |
| ATOM | 630 | OG1 | THR | A | 91 | 81.791 | 194.558 | −13.923 | 1.00 | 50.00 | AAAB | O |
| ATOM | 631 | H | THR | A | 91 | 82.376 | 197.103 | −13.528 | 0.00 | 0.00 | AAAB | H |
| ATOM | 632 | HG1 | THR | A | 91 | 82.158 | 194.104 | −14.674 | 0.00 | 0.00 | AAAB | H |
| ATOM | 633 | N | GLU | A | 92 | 80.863 | 195.576 | −11.243 | 1.00 | 50.00 | AAAB | N |
| ATOM | 634 | CA | GLU | A | 92 | 80.643 | 195.178 | −9.857 | 1.00 | 50.00 | AAAB | C |
| ATOM | 635 | C | GLU | A | 92 | 79.854 | 196.174 | −9.016 | 1.00 | 50.00 | AAAB | C |
| ATOM | 636 | O | GLU | A | 92 | 78.974 | 195.801 | −8.251 | 1.00 | 50.00 | AAAB | O |
| ATOM | 637 | CB | GLU | A | 92 | 81.983 | 194.744 | −9.239 | 1.00 | 50.00 | AAAB | C |
| ATOM | 638 | CG | GLU | A | 92 | 82.519 | 195.618 | −8.096 | 1.00 | 50.00 | AAAB | C |
| ATOM | 639 | CD | GLU | A | 92 | 84.006 | 195.455 | −7.853 | 1.00 | 50.00 | AAAB | C |
| ATOM | 640 | OE1 | GLU | A | 92 | 84.659 | 194.646 | −8.516 | 1.00 | 50.00 | AAAB | O |
| ATOM | 641 | OE2 | GLU | A | 92 | 84.523 | 196.167 | −6.991 | 1.00 | 50.00 | AAAB | O |
| ATOM | 642 | H | GLU | A | 92 | 81.799 | 195.725 | −11.566 | 0.00 | 0.00 | AAAB | H |
| ATOM | 643 | N | LEU | A | 93 | 80.186 | 197.465 | −9.212 | 1.00 | 50.00 | AAAB | N |
| ATOM | 644 | CA | LEU | A | 93 | 79.347 | 198.449 | −8.546 | 1.00 | 50.00 | AAAB | C |
| ATOM | 645 | C | LEU | A | 93 | 78.002 | 198.593 | −9.237 | 1.00 | 50.00 | AAAB | C |
| ATOM | 646 | O | LEU | A | 93 | 77.008 | 198.093 | −8.744 | 1.00 | 50.00 | AAAB | O |
| ATOM | 647 | CB | LEU | A | 93 | 80.095 | 199.776 | −8.347 | 1.00 | 50.00 | AAAB | C |
| ATOM | 648 | CG | LEU | A | 93 | 81.332 | 199.647 | −7.445 | 1.00 | 50.00 | AAAB | C |
| ATOM | 649 | CD1 | LEU | A | 93 | 82.183 | 200.918 | −7.453 | 1.00 | 50.00 | AAAB | C |
| ATOM | 650 | CD2 | LEU | A | 93 | 80.997 | 199.198 | −6.019 | 1.00 | 50.00 | AAAB | C |
| ATOM | 651 | H | LEU | A | 93 | 80.903 | 197.731 | −9.854 | 0.00 | 0.00 | AAAB | H |
| ATOM | 652 | N | GLN | A | 94 | 77.998 | 199.260 | −10.401 | 1.00 | 50.00 | AAAB | N |
| ATOM | 653 | CA | GLN | A | 94 | 76.740 | 199.521 | −11.113 | 1.00 | 50.00 | AAAB | C |
| ATOM | 654 | C | GLN | A | 94 | 75.904 | 198.303 | −11.501 | 1.00 | 50.00 | AAAB | C |
| ATOM | 655 | O | GLN | A | 94 | 74.914 | 198.008 | −10.844 | 1.00 | 50.00 | AAAB | O |
| ATOM | 656 | CB | GLN | A | 94 | 77.018 | 200.423 | −12.311 | 1.00 | 50.00 | AAAB | C |
| ATOM | 657 | CG | GLN | A | 94 | 75.880 | 200.735 | −13.286 | 1.00 | 50.00 | AAAB | C |
| ATOM | 658 | CD | GLN | A | 94 | 76.467 | 201.236 | −14.591 | 1.00 | 50.00 | AAAB | C |
| ATOM | 659 | NE2 | GLN | A | 94 | 76.179 | 202.520 | −14.837 | 1.00 | 50.00 | AAAB | N |
| ATOM | 660 | OE1 | GLN | A | 94 | 77.128 | 200.523 | −15.335 | 1.00 | 50.00 | AAAB | O |
| ATOM | 661 | H | GLN | A | 94 | 78.885 | 199.529 | −10.768 | 0.00 | 0.00 | AAAB | H |
| ATOM | 662 | 1HE2 | GLN | A | 94 | 76.538 | 202.941 | −15.671 | 0.00 | 0.00 | AAAB | H |
| ATOM | 663 | 2HE2 | GLN | A | 94 | 75.636 | 203.071 | −14.206 | 0.00 | 0.00 | AAAB | H |
| ATOM | 664 | N | LEU | A | 95 | 76.325 | 197.622 | −12.592 | 1.00 | 50.00 | AAAB | N |
| ATOM | 665 | CA | LEU | A | 95 | 75.513 | 196.551 | −13.185 | 1.00 | 50.00 | AAAB | C |
| ATOM | 666 | C | LEU | A | 95 | 75.449 | 195.241 | −12.393 | 1.00 | 50.00 | AAAB | C |
| ATOM | 667 | O | LEU | A | 95 | 75.038 | 194.201 | −12.893 | 1.00 | 50.00 | AAAB | O |
| ATOM | 668 | CB | LEU | A | 95 | 75.997 | 196.350 | −14.637 | 1.00 | 50.00 | AAAB | C |
| ATOM | 669 | CG | LEU | A | 95 | 75.209 | 195.382 | −15.537 | 1.00 | 50.00 | AAAB | C |
| ATOM | 670 | CD1 | LEU | A | 95 | 73.735 | 195.776 | −15.667 | 1.00 | 50.00 | AAAB | C |
| ATOM | 671 | CD2 | LEU | A | 95 | 75.878 | 195.179 | −16.897 | 1.00 | 50.00 | AAAB | C |
| ATOM | 672 | H | LEU | A | 95 | 77.170 | 197.894 | −13.053 | 0.00 | 0.00 | AAAB | H |
| ATOM | 673 | N | LEU | A | 96 | 75.889 | 195.320 | −11.126 | 1.00 | 50.00 | AAAB | N |
| ATOM | 674 | CA | LEU | A | 96 | 75.959 | 194.083 | −10.357 | 1.00 | 50.00 | AAAB | C |
| ATOM | 675 | C | LEU | A | 96 | 75.341 | 194.201 | −8.972 | 1.00 | 50.00 | AAAB | C |
| ATOM | 676 | O | LEU | A | 96 | 74.555 | 193.356 | −8.568 | 1.00 | 50.00 | AAAB | O |
| ATOM | 677 | CB | LEU | A | 96 | 77.400 | 193.562 | −10.250 | 1.00 | 50.00 | AAAB | C |
| ATOM | 678 | CG | LEU | A | 96 | 77.918 | 192.570 | −11.315 | 1.00 | 50.00 | AAAB | C |
| ATOM | 679 | CD1 | LEU | A | 96 | 78.021 | 193.083 | −12.756 | 1.00 | 50.00 | AAAB | C |
| ATOM | 680 | CD2 | LEU | A | 96 | 79.271 | 192.002 | −10.887 | 1.00 | 50.00 | AAAB | C |
| ATOM | 681 | H | LEU | A | 96 | 76.138 | 196.193 | −10.712 | 0.00 | 0.00 | AAAB | H |
| ATOM | 682 | N | MET | A | 97 | 75.737 | 195.264 | −8.244 | 1.00 | 50.00 | AAAB | N |
| ATOM | 683 | CA | MET | A | 97 | 75.378 | 195.251 | −6.821 | 1.00 | 50.00 | AAAB | C |
| ATOM | 684 | C | MET | A | 97 | 74.623 | 196.452 | −6.302 | 1.00 | 50.00 | AAAB | C |
| ATOM | 685 | O | MET | A | 97 | 73.757 | 196.370 | −5.440 | 1.00 | 50.00 | AAAB | O |
| ATOM | 686 | CB | MET | A | 97 | 76.617 | 195.050 | −5.956 | 1.00 | 50.00 | AAAB | C |
| ATOM | 687 | CG | MET | A | 97 | 77.147 | 193.624 | −6.039 | 1.00 | 50.00 | AAAB | C |
| ATOM | 688 | SD | MET | A | 97 | 78.530 | 193.331 | −4.934 | 1.00 | 50.00 | AAAB | S |
| ATOM | 689 | CE | MET | A | 97 | 78.734 | 191.584 | −5.304 | 1.00 | 50.00 | AAAB | C |
| ATOM | 690 | H | MET | A | 97 | 76.336 | 195.971 | −8.619 | 0.00 | 0.00 | AAAB | H |
| ATOM | 691 | N | GLN | A | 98 | 75.032 | 197.594 | −6.857 | 1.00 | 50.00 | AAAB | N |
| ATOM | 692 | CA | GLN | A | 98 | 74.505 | 198.831 | −6.329 | 1.00 | 50.00 | AAAB | C |
| ATOM | 693 | C | GLN | A | 98 | 73.093 | 199.151 | −6.782 | 1.00 | 50.00 | AAAB | C |
| ATOM | 694 | O | GLN | A | 98 | 72.184 | 199.217 | −5.971 | 1.00 | 50.00 | AAAB | O |
| ATOM | 695 | CB | GLN | A | 98 | 75.547 | 199.954 | −6.473 | 1.00 | 50.00 | AAAB | C |
| ATOM | 696 | CG | GLN | A | 98 | 75.652 | 200.656 | −7.814 | 1.00 | 50.00 | AAAB | C |
| ATOM | 697 | CD | GLN | A | 98 | 76.691 | 201.734 | −7.739 | 1.00 | 50.00 | AAAB | C |
| ATOM | 698 | NE2 | GLN | A | 98 | 76.327 | 202.771 | −8.474 | 1.00 | 50.00 | AAAB | N |
| ATOM | 699 | OE1 | GLN | A | 98 | 77.703 | 201.677 | −7.053 | 1.00 | 50.00 | AAAB | O |
| ATOM | 700 | H | GLN | A | 98 | 75.706 | 197.560 | −7.581 | 0.00 | 0.00 | AAAB | H |
| ATOM | 701 | 1HE2 | GLN | A | 98 | 76.795 | 203.644 | −8.385 | 0.00 | 0.00 | AAAB | H |
| ATOM | 702 | 2HE2 | GLN | A | 98 | 75.560 | 202.673 | −9.107 | 0.00 | 0.00 | AAAB | H |
| ATOM | 703 | N | SER | A | 99 | 72.915 | 199.285 | −8.106 | 1.00 | 50.00 | AAAB | N |
| ATOM | 704 | CA | SER | A | 99 | 71.547 | 199.483 | −8.568 | 1.00 | 50.00 | AAAB | C |
| ATOM | 705 | C | SER | A | 99 | 70.720 | 198.211 | −8.577 | 1.00 | 50.00 | AAAB | C |
| ATOM | 706 | O | SER | A | 99 | 69.508 | 198.239 | −8.749 | 1.00 | 50.00 | AAAB | O |
| ATOM | 707 | CB | SER | A | 99 | 71.560 | 200.145 | −9.944 | 1.00 | 50.00 | AAAB | C |
| ATOM | 708 | OG | SER | A | 99 | 72.389 | 199.414 | −10.853 | 1.00 | 50.00 | AAAB | O |
| ATOM | 709 | H | SER | A | 99 | 73.643 | 199.110 | −8.767 | 0.00 | 0.00 | AAAB | H |
| ATOM | 710 | HG | SER | A | 99 | 72.259 | 199.814 | −11.703 | 0.00 | 0.00 | AAAB | H |
| ATOM | 711 | N | THR | A | 100 | 71.458 | 197.088 | −8.400 | 1.00 | 50.00 | AAAB | N |
| ATOM | 712 | CA | THR | A | 100 | 70.855 | 195.756 | −8.400 | 1.00 | 50.00 | AAAB | C |
| ATOM | 713 | C | THR | A | 100 | 70.036 | 195.487 | −9.653 | 1.00 | 50.00 | AAAB | C |
| ATOM | 714 | O | THR | A | 100 | 68.936 | 194.947 | −9.620 | 1.00 | 50.00 | AAAB | O |
| ATOM | 715 | CB | THR | A | 100 | 70.051 | 195.513 | −7.110 | 1.00 | 50.00 | AAAB | C |
| ATOM | 716 | CG2 | THR | A | 100 | 69.812 | 194.024 | −6.833 | 1.00 | 50.00 | AAAB | C |
| ATOM | 717 | OG1 | THR | A | 100 | 70.717 | 196.103 | −5.989 | 1.00 | 50.00 | AAAB | O |
| ATOM | 718 | H | THR | A | 100 | 72.439 | 197.171 | −8.233 | 0.00 | 0.00 | AAAB | H |
| ATOM | 719 | HG1 | THR | A | 100 | 70.164 | 195.929 | −5.239 | 0.00 | 0.00 | AAAB | H |
| ATOM | 720 | N | GLN | A | 101 | 70.647 | 195.937 | −10.777 | 1.00 | 50.00 | AAAB | N |
| ATOM | 721 | CA | GLN | A | 101 | 70.051 | 195.814 | −12.115 | 1.00 | 50.00 | AAAB | C |
| ATOM | 722 | C | GLN | A | 101 | 68.577 | 196.222 | −12.246 | 1.00 | 50.00 | AAAB | C |
| ATOM | 723 | CB | GLN | A | 101 | 70.394 | 194.436 | −12.725 | 1.00 | 50.00 | AAAB | C |
| ATOM | 724 | CG | GLN | A | 101 | 69.748 | 193.257 | −11.990 | 1.00 | 50.00 | AAAB | C |
| ATOM | 725 | CD | GLN | A | 101 | 70.186 | 191.928 | −12.540 | 1.00 | 50.00 | AAAB | C |
| ATOM | 726 | NE2 | GLN | A | 101 | 69.144 | 191.125 | −12.799 | 1.00 | 50.00 | AAAB | N |
| ATOM | 727 | OE1 | GLN | A | 101 | 71.363 | 191.625 | −12.681 | 1.00 | 50.00 | AAAB | O |
| ATOM | 728 | 1OCT | GLN | A | 101 | 67.867 | 195.673 | −13.091 | 1.00 | 50.00 | AAAB | O |
| ATOM | 729 | 2OCT | GLN | A | 101 | 68.140 | 197.097 | −11.498 | 1.00 | 99.99 | AAAB | O |
| ATOM | 730 | H | GLN | A | 101 | 71.524 | 196.403 | −10.676 | 0.00 | 0.00 | AAAB | H |
| ATOM | 731 | 1HE2 | GLN | A | 101 | 69.280 | 190.185 | −13.104 | 0.00 | 0.00 | AAAB | H |
| ATOM | 732 | 2HE2 | GLN | A | 101 | 68.210 | 191.465 | −12.675 | 0.00 | 0.00 | AAAB | H |
| END | | | | | | | | | | | | |

F1 Model Coordinantes (SEQ ID NO: 43)

| | | | | | | | | | | | | |
|---|---|---|---|---|---|---|---|---|---|---|---|---|
| ATOM | 1 | N | PHE | A | 1 | 66.925 | 202.269 | −9.881 | 1.00 | 99.99 | AAAA | N |
| ATOM | 2 | CA | PHE | A | 1 | 67.235 | 202.956 | −8.617 | 1.00 | 99.99 | AAAA | C |
| ATOM | 3 | C | PHE | A | 1 | 68.654 | 202.705 | −8.134 | 1.00 | 99.99 | AAAA | C |
| ATOM | 4 | O | PHE | A | 1 | 69.249 | 201.656 | −8.335 | 1.00 | 99.99 | AAAA | O |
| ATOM | 5 | CB | PHE | A | 1 | 66.205 | 202.607 | −7.527 | 1.00 | 99.99 | AAAA | C |
| ATOM | 6 | CG | PHE | A | 1 | 66.228 | 201.125 | −7.211 | 1.00 | 99.99 | AAAA | C |
| ATOM | 7 | CD1 | PHE | A | 1 | 65.439 | 200.238 | −7.976 | 1.00 | 99.99 | AAAA | C |
| ATOM | 8 | CD2 | PHE | A | 1 | 67.053 | 200.650 | −6.167 | 1.00 | 99.99 | AAAA | C |
| ATOM | 9 | CE1 | PHE | A | 1 | 65.500 | 198.856 | −7.716 | 1.00 | 99.99 | AAAA | C |
| ATOM | 10 | CE2 | PHE | A | 1 | 67.118 | 199.268 | −5.907 | 1.00 | 99.99 | AAAA | C |
| ATOM | 11 | CZ | PHE | A | 1 | 66.349 | 198.385 | −6.693 | 1.00 | 99.99 | AAAA | C |
| ATOM | 12 | 1H | PHE | A | 1 | 65.966 | 202.539 | −10.184 | 0.00 | 0.00 | AAAA | H |
| ATOM | 13 | 2H | PHE | A | 1 | 66.973 | 201.238 | −9.753 | 0.00 | 0.00 | AAAA | H |
| ATOM | 14 | 3H | PHE | A | 1 | 67.605 | 202.576 | −10.606 | 0.00 | 0.00 | AAAA | H |
| ATOM | 15 | N | LEU | A | 2 | 69.143 | 203.746 | −7.438 | 1.00 | 99.99 | AAAA | N |
| ATOM | 16 | CA | LEU | A | 2 | 70.395 | 203.651 | −6.690 | 1.00 | 99.99 | AAAA | C |
| ATOM | 17 | C | LEU | A | 2 | 70.328 | 202.738 | −5.478 | 1.00 | 99.99 | AAAA | C |
| ATOM | 18 | O | LEU | A | 2 | 69.380 | 202.773 | −4.706 | 1.00 | 99.99 | AAAA | O |
| ATOM | 19 | CB | LEU | A | 2 | 70.746 | 205.070 | −6.248 | 1.00 | 99.99 | AAAA | C |
| ATOM | 20 | CG | LEU | A | 2 | 72.159 | 205.409 | −5.766 | 1.00 | 99.99 | AAAA | C |
| ATOM | 21 | CD1 | LEU | A | 2 | 72.288 | 206.912 | −5.898 | 1.00 | 99.99 | AAAA | C |
| ATOM | 22 | CD2 | LEU | A | 2 | 72.565 | 204.962 | −4.359 | 1.00 | 99.99 | AAAA | C |
| ATOM | 23 | H | LEU | A | 2 | 68.616 | 204.595 | −7.418 | 0.00 | 0.00 | AAAA | H |
| ATOM | 24 | N | GLY | A | 3 | 71.432 | 201.992 | −5.312 | 1.00 | 99.99 | AAAA | N |
| ATOM | 25 | CA | GLY | A | 3 | 71.709 | 201.409 | −4.001 | 1.00 | 99.99 | AAAA | C |
| ATOM | 26 | C | GLY | A | 3 | 73.192 | 201.111 | −3.902 | 1.00 | 99.99 | AAAA | C |
| ATOM | 27 | O | GLY | A | 3 | 73.991 | 201.687 | −4.633 | 1.00 | 99.99 | AAAA | O |
| ATOM | 28 | H | GLY | A | 3 | 72.096 | 201.882 | −6.052 | 0.00 | 0.00 | AAAA | H |
| ATOM | 29 | N | PHE | A | 4 | 73.500 | 200.160 | −2.995 | 1.00 | 99.99 | AAAA | N |
| ATOM | 30 | CA | PHE | A | 4 | 74.810 | 199.500 | −2.951 | 1.00 | 99.99 | AAAA | C |
| ATOM | 31 | C | PHE | A | 4 | 74.764 | 198.217 | −2.128 | 1.00 | 99.99 | AAAA | C |
| ATOM | 32 | O | PHE | A | 4 | 73.892 | 198.047 | −1.288 | 1.00 | 99.99 | AAAA | O |
| ATOM | 33 | CB | PHE | A | 4 | 75.908 | 200.449 | −2.434 | 1.00 | 99.99 | AAAA | C |
| ATOM | 34 | CG | PHE | A | 4 | 75.547 | 200.981 | −1.066 | 1.00 | 99.99 | AAAA | C |
| ATOM | 35 | CD1 | PHE | A | 4 | 74.742 | 202.138 | −0.959 | 1.00 | 99.99 | AAAA | C |
| ATOM | 36 | CD2 | PHE | A | 4 | 76.000 | 200.295 | 0.081 | 1.00 | 99.99 | AAAA | C |
| ATOM | 37 | CE1 | PHE | A | 4 | 74.336 | 202.581 | 0.313 | 1.00 | 99.99 | AAAA | C |
| ATOM | 38 | CE2 | PHE | A | 4 | 75.597 | 200.739 | 1.354 | 1.00 | 99.99 | AAAA | C |
| ATOM | 39 | CZ | PHE | A | 4 | 74.752 | 201.865 | 1.455 | 1.00 | 99.99 | AAAA | C |
| ATOM | 40 | H | PHE | A | 4 | 72.802 | 199.905 | −2.326 | 0.00 | 0.00 | AAAA | H |
| ATOM | 41 | N | LEU | A | 5 | 75.751 | 197.337 | −2.390 | 1.00 | 99.99 | AAAA | N |
| ATOM | 42 | CA | LEU | A | 5 | 75.840 | 196.122 | −1.567 | 1.00 | 99.99 | AAAA | C |
| ATOM | 43 | C | LEU | A | 5 | 77.130 | 196.048 | −0.755 | 1.00 | 99.99 | AAAA | C |
| ATOM | 44 | O | LEU | A | 5 | 77.190 | 195.486 | 0.332 | 1.00 | 99.99 | AAAA | O |
| ATOM | 45 | CB | LEU | A | 5 | 75.617 | 194.896 | −2.469 | 1.00 | 99.99 | AAAA | C |
| ATOM | 46 | CG | LEU | A | 5 | 75.457 | 193.510 | −1.819 | 1.00 | 99.99 | AAAA | C |
| ATOM | 47 | CD1 | LEU | A | 5 | 74.588 | 192.598 | −2.687 | 1.00 | 99.99 | AAAA | C |
| ATOM | 48 | CD2 | LEU | A | 5 | 76.792 | 192.820 | −1.516 | 1.00 | 99.99 | AAAA | C |
| ATOM | 49 | H | LEU | A | 5 | 76.396 | 197.502 | −3.137 | 0.00 | 0.00 | AAAA | H |
| ATOM | 50 | N | LEU | A | 6 | 78.174 | 196.674 | −1.338 | 1.00 | 99.99 | AAAA | N |
| ATOM | 51 | CA | LEU | A | 6 | 79.471 | 196.710 | −0.663 | 1.00 | 99.99 | AAAA | C |
| ATOM | 52 | C | LEU | A | 6 | 79.490 | 197.717 | 0.477 | 1.00 | 99.99 | AAAA | C |
| ATOM | 53 | O | LEU | A | 6 | 79.079 | 198.862 | 0.336 | 1.00 | 99.99 | AAAA | O |
| ATOM | 54 | CB | LEU | A | 6 | 80.546 | 196.978 | −1.733 | 1.00 | 99.99 | AAAA | C |
| ATOM | 55 | CG | LEU | A | 6 | 82.029 | 196.930 | −1.323 | 1.00 | 99.99 | AAAA | C |
| ATOM | 56 | CD1 | LEU | A | 6 | 82.904 | 196.562 | −2.525 | 1.00 | 99.99 | AAAA | C |
| ATOM | 57 | CD2 | LEU | A | 6 | 82.536 | 198.236 | −0.707 | 1.00 | 99.99 | AAAA | C |
| ATOM | 58 | H | LEU | A | 6 | 78.033 | 197.191 | −2.180 | 0.00 | 0.00 | AAAA | H |
| ATOM | 59 | N | GLY | A | 7 | 79.987 | 197.219 | 1.620 | 1.00 | 99.99 | AAAA | N |
| ATOM | 60 | CA | GLY | A | 7 | 80.157 | 198.098 | 2.772 | 1.00 | 99.99 | AAAA | C |
| ATOM | 61 | C | GLY | A | 7 | 81.625 | 198.372 | 3.008 | 1.00 | 99.99 | AAAA | C |
| ATOM | 62 | O | GLY | A | 7 | 82.482 | 197.666 | 2.492 | 1.00 | 99.99 | AAAA | O |
| ATOM | 63 | H | GLY | A | 7 | 80.325 | 196.279 | 1.645 | 0.00 | 0.00 | AAAA | H |
| ATOM | 64 | N | VAL | A | 8 | 81.869 | 199.443 | 3.800 | 1.00 | 99.99 | AAAA | N |
| ATOM | 65 | CA | VAL | A | 8 | 83.240 | 199.925 | 4.032 | 1.00 | 99.99 | AAAA | C |
| ATOM | 66 | C | VAL | A | 8 | 83.823 | 200.547 | 2.748 | 1.00 | 99.99 | AAAA | C |
| ATOM | 67 | O | VAL | A | 8 | 83.170 | 200.572 | 1.711 | 1.00 | 99.99 | AAAA | O |
| ATOM | 68 | CB | VAL | A | 8 | 84.094 | 198.822 | 4.736 | 1.00 | 99.99 | AAAA | C |
| ATOM | 69 | CG1 | VAL | A | 8 | 85.504 | 199.210 | 5.203 | 1.00 | 99.99 | AAAA | C |
| ATOM | 70 | CG2 | VAL | A | 8 | 83.332 | 198.320 | 5.967 | 1.00 | 99.99 | AAAA | C |
| ATOM | 71 | H | VAL | A | 8 | 81.088 | 199.945 | 4.170 | 0.00 | 0.00 | AAAA | H |
| ATOM | 72 | N | GLY | A | 9 | 85.028 | 201.135 | 2.868 | 1.00 | 99.99 | AAAA | N |
| ATOM | 73 | CA | GLY | A | 9 | 85.441 | 202.103 | 1.865 | 1.00 | 99.99 | AAAA | C |
| ATOM | 74 | C | GLY | A | 9 | 85.413 | 203.461 | 2.523 | 1.00 | 99.99 | AAAA | C |
| ATOM | 75 | O | GLY | A | 9 | 86.348 | 204.249 | 2.446 | 1.00 | 99.99 | AAAA | O |
| ATOM | 76 | H | GLY | A | 9 | 85.585 | 201.069 | 3.693 | 0.00 | 0.00 | AAAA | H |
| ATOM | 77 | N | SER | A | 10 | 84.264 | 203.671 | 3.216 | 1.00 | 99.99 | AAAA | N |
| ATOM | 78 | CA | SER | A | 10 | 84.030 | 204.874 | 4.019 | 1.00 | 99.99 | AAAA | C |
| ATOM | 79 | C | SER | A | 10 | 84.116 | 206.172 | 3.223 | 1.00 | 99.99 | AAAA | C |
| ATOM | 80 | O | SER | A | 10 | 84.288 | 207.264 | 3.749 | 1.00 | 99.99 | AAAA | O |
| ATOM | 81 | CB | SER | A | 10 | 84.950 | 204.854 | 5.251 | 1.00 | 99.99 | AAAA | C |
| ATOM | 82 | OG | SER | A | 10 | 84.475 | 205.747 | 6.260 | 1.00 | 99.99 | AAAA | O |
| ATOM | 83 | H | SER | A | 10 | 83.555 | 202.966 | 3.190 | 0.00 | 0.00 | AAAA | H |
| ATOM | 84 | HG | SER | A | 10 | 85.185 | 205.864 | 6.879 | 0.00 | 0.00 | AAAA | H |
| ATOM | 85 | N | ALA | A | 11 | 84.019 | 205.961 | 1.896 | 1.00 | 99.99 | AAAA | N |
| ATOM | 86 | CA | ALA | A | 11 | 84.234 | 207.042 | 0.953 | 1.00 | 99.99 | AAAA | C |
| ATOM | 87 | C | ALA | A | 11 | 82.898 | 207.398 | 0.359 | 1.00 | 99.99 | AAAA | C |
| ATOM | 88 | O | ALA | A | 11 | 82.431 | 208.505 | 0.572 | 1.00 | 99.99 | AAAA | O |
| ATOM | 89 | CB | ALA | A | 11 | 85.222 | 206.615 | −0.134 | 1.00 | 99.99 | AAAA | C |
| ATOM | 90 | H | ALA | A | 11 | 83.741 | 205.058 | 1.571 | 0.00 | 0.00 | AAAA | H |
| ATOM | 91 | N | ILE | A | 12 | 82.296 | 206.358 | −0.289 | 1.00 | 50.00 | AAAA | N |
| ATOM | 92 | CA | ILE | A | 12 | 80.850 | 206.126 | −0.500 | 1.00 | 50.00 | AAAA | C |
| ATOM | 93 | C | ILE | A | 12 | 80.037 | 207.088 | −1.360 | 1.00 | 50.00 | AAAA | C |
| ATOM | 94 | O | ILE | A | 12 | 79.239 | 206.671 | −2.187 | 1.00 | 50.00 | AAAA | O |
| ATOM | 95 | CB | ILE | A | 12 | 80.110 | 205.756 | 0.811 | 1.00 | 50.00 | AAAA | C |
| ATOM | 96 | CG1 | ILE | A | 12 | 79.954 | 206.944 | 1.775 | 1.00 | 50.00 | AAAA | C |
| ATOM | 97 | CG2 | ILE | A | 12 | 80.835 | 204.572 | 1.467 | 1.00 | 50.00 | AAAA | C |
| ATOM | 98 | CD1 | ILE | A | 12 | 79.573 | 206.592 | 3.212 | 1.00 | 50.00 | AAAA | C |
| ATOM | 99 | H | ILE | A | 12 | 82.904 | 205.609 | −0.550 | 0.00 | 0.00 | AAAA | H |
| ATOM | 100 | N | ALA | A | 13 | 80.303 | 208.393 | −1.157 | 1.00 | 50.00 | AAAA | N |
| ATOM | 101 | CA | ALA | A | 13 | 79.650 | 209.508 | −1.838 | 1.00 | 50.00 | AAAA | C |
| ATOM | 102 | C | ALA | A | 13 | 79.807 | 209.434 | −3.340 | 1.00 | 50.00 | AAAA | C |
| ATOM | 103 | O | ALA | A | 13 | 78.872 | 209.668 | −4.093 | 1.00 | 50.00 | AAAA | O |
| ATOM | 104 | CB | ALA | A | 13 | 80.261 | 210.821 | −1.352 | 1.00 | 50.00 | AAAA | C |
| ATOM | 105 | H | ALA | A | 13 | 80.949 | 208.629 | −0.442 | 0.00 | 0.00 | AAAA | H |
| ATOM | 106 | N | SER | A | 14 | 81.039 | 209.032 | −3.731 | 1.00 | 50.00 | AAAA | N |
| ATOM | 107 | CA | SER | A | 14 | 81.259 | 208.740 | −5.144 | 1.00 | 50.00 | AAAA | C |
| ATOM | 108 | C | SER | A | 14 | 80.357 | 207.639 | −5.676 | 1.00 | 50.00 | AAAA | C |
| ATOM | 109 | O | SER | A | 14 | 79.776 | 207.797 | −6.734 | 1.00 | 50.00 | AAAA | O |
| ATOM | 110 | CB | SER | A | 14 | 82.739 | 208.432 | −5.413 | 1.00 | 50.00 | AAAA | C |
| ATOM | 111 | OG | SER | A | 14 | 82.944 | 208.150 | −6.804 | 1.00 | 50.00 | AAAA | O |
| ATOM | 112 | H | SER | A | 14 | 81.766 | 208.893 | −3.057 | 0.00 | 0.00 | AAAA | H |
| ATOM | 113 | HG | SER | A | 14 | 83.876 | 208.206 | −6.978 | 0.00 | 0.00 | AAAA | H |
| ATOM | 114 | N | GLY | A | 15 | 80.244 | 206.550 | −4.881 | 1.00 | 50.00 | AAAA | N |
| ATOM | 115 | CA | GLY | A | 15 | 79.411 | 205.396 | −5.249 | 1.00 | 50.00 | AAAA | C |
| ATOM | 116 | C | GLY | A | 15 | 77.927 | 205.694 | −5.411 | 1.00 | 50.00 | AAAA | C |
| ATOM | 117 | O | GLY | A | 15 | 77.253 | 205.221 | −6.319 | 1.00 | 50.00 | AAAA | O |
| ATOM | 118 | H | GLY | A | 15 | 80.741 | 206.545 | −4.016 | 0.00 | 0.00 | AAAA | H |
| ATOM | 119 | N | VAL | A | 16 | 77.465 | 206.555 | −4.491 | 1.00 | 50.00 | AAAA | N |
| ATOM | 120 | CA | VAL | A | 16 | 76.116 | 207.106 | −4.590 | 1.00 | 50.00 | AAAA | C |
| ATOM | 121 | C | VAL | A | 16 | 75.959 | 208.005 | −5.814 | 1.00 | 50.00 | AAAA | C |
| ATOM | 122 | O | VAL | A | 16 | 74.958 | 207.990 | −6.513 | 1.00 | 50.00 | AAAA | O |
| ATOM | 123 | CB | VAL | A | 16 | 75.772 | 207.828 | −3.272 | 1.00 | 50.00 | AAAA | C |
| ATOM | 124 | CG1 | VAL | A | 16 | 74.424 | 208.552 | −3.295 | 1.00 | 50.00 | AAAA | C |
| ATOM | 125 | CG2 | VAL | A | 16 | 75.841 | 206.844 | −2.102 | 1.00 | 50.00 | AAAA | C |
| ATOM | 126 | H | VAL | A | 16 | 78.063 | 206.848 | −3.747 | 0.00 | 0.00 | AAAA | H |
| ATOM | 127 | N | ALA | A | 17 | 77.033 | 208.760 | −6.102 | 1.00 | 50.00 | AAAA | N |
| ATOM | 128 | CA | ALA | A | 17 | 76.968 | 209.524 | −7.345 | 1.00 | 50.00 | AAAA | C |
| ATOM | 129 | C | ALA | A | 17 | 76.939 | 208.655 | −8.596 | 1.00 | 50.00 | AAAA | C |
| ATOM | 130 | O | ALA | A | 17 | 76.292 | 208.993 | −9.575 | 1.00 | 50.00 | AAAA | O |
| ATOM | 131 | CB | ALA | A | 17 | 78.121 | 210.526 | −7.443 | 1.00 | 50.00 | AAAA | C |
| ATOM | 132 | H | ALA | A | 17 | 77.868 | 208.726 | −5.552 | 0.00 | 0.00 | AAAA | H |
| ATOM | 133 | N | VAL | A | 18 | 77.631 | 207.500 | −8.486 | 1.00 | 50.00 | AAAA | N |
| ATOM | 134 | CA | VAL | A | 18 | 77.688 | 206.497 | −9.555 | 1.00 | 50.00 | AAAA | C |
| ATOM | 135 | C | VAL | A | 18 | 76.319 | 206.067 | −10.081 | 1.00 | 50.00 | AAAA | C |
| ATOM | 136 | O | VAL | A | 18 | 76.047 | 206.125 | −11.273 | 1.00 | 50.00 | AAAA | O |
| ATOM | 137 | CB | VAL | A | 18 | 78.553 | 205.279 | −9.115 | 1.00 | 50.00 | AAAA | C |
| ATOM | 138 | CG1 | VAL | A | 18 | 78.560 | 204.103 | −10.102 | 1.00 | 50.00 | AAAA | C |
| ATOM | 139 | CG2 | VAL | A | 18 | 79.999 | 205.683 | −8.829 | 1.00 | 50.00 | AAAA | C |
| ATOM | 140 | H | VAL | A | 18 | 78.117 | 207.351 | −7.629 | 0.00 | 0.00 | AAAA | H |
| ATOM | 141 | N | SER | A | 19 | 75.465 | 205.646 | −9.131 | 1.00 | 50.00 | AAAA | N |
| ATOM | 142 | CA | SER | A | 19 | 74.146 | 205.157 | −9.543 | 1.00 | 50.00 | AAAA | C |
| ATOM | 143 | C | SER | A | 19 | 73.116 | 206.242 | −9.806 | 1.00 | 50.00 | AAAA | C |
| ATOM | 144 | O | SER | A | 19 | 72.112 | 206.038 | −10.473 | 1.00 | 50.00 | AAAA | O |
| ATOM | 145 | CB | SER | A | 19 | 73.624 | 204.177 | −8.498 | 1.00 | 50.00 | AAAA | C |
| ATOM | 146 | OG | SER | A | 19 | 72.721 | 203.225 | −9.061 | 1.00 | 50.00 | AAAA | O |
| ATOM | 147 | H | SER | A | 19 | 75.730 | 205.695 | −8.168 | 0.00 | 0.00 | AAAA | H |
| ATOM | 148 | HG | SER | A | 19 | 72.388 | 202.694 | −8.348 | 0.00 | 0.00 | AAAA | H |
| ATOM | 149 | N | LYS | A | 20 | 73.422 | 207.435 | −9.268 | 1.00 | 50.00 | AAAA | N |
| ATOM | 150 | CA | LYS | A | 20 | 72.561 | 208.571 | −9.593 | 1.00 | 50.00 | AAAA | C |
| ATOM | 151 | C | LYS | A | 20 | 72.714 | 209.004 | −11.043 | 1.00 | 50.00 | AAAA | C |
| ATOM | 152 | O | LYS | A | 20 | 71.762 | 209.281 | −11.765 | 1.00 | 50.00 | AAAA | O |
| ATOM | 153 | CB | LYS | A | 20 | 72.866 | 209.725 | −8.642 | 1.00 | 50.00 | AAAA | C |
| ATOM | 154 | CG | LYS | A | 20 | 71.928 | 210.904 | −8.878 | 1.00 | 50.00 | AAAA | C |
| ATOM | 155 | CD | LYS | A | 20 | 72.280 | 212.116 | −8.033 | 1.00 | 50.00 | AAAA | C |
| ATOM | 156 | CE | LYS | A | 20 | 71.384 | 213.291 | −8.407 | 1.00 | 50.00 | AAAA | C |
| ATOM | 157 | NZ | LYS | A | 20 | 71.721 | 214.432 | −7.553 | 1.00 | 50.00 | AAAA | N |
| ATOM | 158 | H | LYS | A | 20 | 74.255 | 207.571 | −8.730 | 0.00 | 0.00 | AAAA | H |
| ATOM | 159 | 1HZ | LYS | A | 20 | 71.141 | 215.248 | −7.834 | 0.00 | 0.00 | AAAA | H |
| ATOM | 160 | 2HZ | LYS | A | 20 | 72.729 | 214.663 | −7.673 | 0.00 | 0.00 | AAAA | H |
| ATOM | 161 | 3HZ | LYS | A | 20 | 71.531 | 214.182 | −6.562 | 0.00 | 0.00 | AAAA | H |
| ATOM | 162 | N | VAL | A | 21 | 73.997 | 208.999 | −11.444 | 1.00 | 50.00 | AAAA | N |
| ATOM | 163 | CA | VAL | A | 21 | 74.258 | 209.361 | −12.829 | 1.00 | 50.00 | AAAA | C |
| ATOM | 164 | C | VAL | A | 21 | 73.928 | 208.269 | −13.833 | 1.00 | 50.00 | AAAA | C |
| ATOM | 165 | O | VAL | A | 21 | 73.998 | 208.505 | −15.027 | 1.00 | 50.00 | AAAA | O |
| ATOM | 166 | CB | VAL | A | 21 | 75.694 | 209.889 | −13.026 | 1.00 | 50.00 | AAAA | C |
| ATOM | 167 | CG1 | VAL | A | 21 | 75.944 | 211.128 | −12.165 | 1.00 | 50.00 | AAAA | C |
| ATOM | 168 | CG2 | VAL | A | 21 | 76.769 | 208.823 | −12.798 | 1.00 | 50.00 | AAAA | C |
| ATOM | 169 | H | VAL | A | 21 | 74.736 | 208.700 | −10.841 | 0.00 | 0.00 | AAAA | H |
| ATOM | 170 | N | LEU | A | 22 | 73.556 | 207.076 | −13.303 | 1.00 | 50.00 | AAAA | N |
| ATOM | 171 | CA | LEU | A | 22 | 73.297 | 205.897 | −14.140 | 1.00 | 50.00 | AAAA | C |
| ATOM | 172 | C | LEU | A | 22 | 72.443 | 206.165 | −15.368 | 1.00 | 50.00 | AAAA | C |
| ATOM | 173 | O | LEU | A | 22 | 72.781 | 205.752 | −16.470 | 1.00 | 50.00 | AAAA | O |
| ATOM | 174 | CB | LEU | A | 22 | 72.711 | 204.773 | −13.266 | 1.00 | 50.00 | AAAA | C |
| ATOM | 175 | CG | LEU | A | 22 | 72.402 | 203.400 | −13.888 | 1.00 | 50.00 | AAAA | C |
| ATOM | 176 | CD1 | LEU | A | 22 | 72.477 | 202.319 | −12.815 | 1.00 | 50.00 | AAAA | C |
| ATOM | 177 | CD2 | LEU | A | 22 | 71.045 | 203.319 | −14.595 | 1.00 | 50.00 | AAAA | C |
| ATOM | 178 | H | LEU | A | 22 | 73.463 | 206.990 | −12.313 | 0.00 | 0.00 | AAAA | H |
| ATOM | 179 | N | HIS | A | 23 | 71.343 | 206.905 | −15.120 | 1.00 | 50.00 | AAAA | N |
| ATOM | 180 | CA | HIS | A | 23 | 70.439 | 207.255 | −16.219 | 1.00 | 50.00 | AAAA | C |
| ATOM | 181 | C | HIS | A | 23 | 71.110 | 208.005 | −17.364 | 1.00 | 50.00 | AAAA | C |
| ATOM | 182 | O | HIS | A | 23 | 71.041 | 207.599 | −18.516 | 1.00 | 50.00 | AAAA | O |
| ATOM | 183 | CB | HIS | A | 23 | 69.234 | 208.030 | −15.659 | 1.00 | 50.00 | AAAA | C |
| ATOM | 184 | CG | HIS | A | 23 | 68.174 | 208.349 | −16.706 | 1.00 | 50.00 | AAAA | C |
| ATOM | 185 | CD2 | HIS | A | 23 | 67.885 | 207.692 | −17.911 | 1.00 | 50.00 | AAAA | C |
| ATOM | 186 | ND1 | HIS | A | 23 | 67.317 | 209.382 | −16.580 | 1.00 | 50.00 | AAAA | N |
| ATOM | 187 | CE1 | HIS | A | 23 | 66.502 | 209.382 | −17.682 | 1.00 | 50.00 | AAAA | C |
| ATOM | 188 | NE2 | HIS | A | 23 | 66.850 | 208.343 | −18.503 | 1.00 | 50.00 | AAAA | N |
| ATOM | 189 | H | HIS | A | 23 | 71.168 | 207.198 | −14.180 | 0.00 | 0.00 | AAAA | H |
| ATOM | 190 | HD1 | HIS | A | 23 | 67.287 | 210.005 | −15.823 | 0.00 | 0.00 | AAAA | H |
| ATOM | 191 | N | LEU | A | 24 | 71.775 | 209.112 | −16.983 | 1.00 | 50.00 | AAAA | N |
| ATOM | 192 | CA | LEU | A | 24 | 72.455 | 209.936 | −17.990 | 1.00 | 50.00 | AAAA | C |
| ATOM | 193 | C | LEU | A | 24 | 73.553 | 209.203 | −18.737 | 1.00 | 50.00 | AAAA | C |
| ATOM | 194 | O | LEU | A | 24 | 73.806 | 209.365 | −19.918 | 1.00 | 50.00 | AAAA | O |
| ATOM | 195 | CB | LEU | A | 24 | 73.057 | 211.193 | −17.360 | 1.00 | 50.00 | AAAA | C |
| ATOM | 196 | CG | LEU | A | 24 | 72.054 | 212.152 | −16.715 | 1.00 | 50.00 | AAAA | C |
| ATOM | 197 | CD1 | LEU | A | 24 | 72.778 | 213.274 | −15.976 | 1.00 | 50.00 | AAAA | C |
| ATOM | 198 | CD2 | LEU | A | 24 | 71.036 | 212.704 | −17.709 | 1.00 | 50.00 | AAAA | C |
| ATOM | 199 | H | LEU | A | 24 | 71.865 | 209.291 | −16.005 | 0.00 | 0.00 | AAAA | H |
| ATOM | 200 | N | GLU | A | 25 | 74.204 | 208.348 | −17.962 | 1.00 | 50.00 | AAAA | N |
| ATOM | 201 | CA | GLU | A | 25 | 75.321 | 207.611 | −18.522 | 1.00 | 50.00 | AAAA | C |
| ATOM | 202 | C | GLU | A | 25 | 74.936 | 206.468 | −19.443 | 1.00 | 50.00 | AAAA | C |
| ATOM | 203 | O | GLU | A | 25 | 75.643 | 206.101 | −20.377 | 1.00 | 50.00 | AAAA | O |
| ATOM | 204 | CB | GLU | A | 25 | 76.143 | 207.209 | −17.324 | 1.00 | 50.00 | AAAA | C |
| ATOM | 205 | CG | GLU | A | 25 | 76.553 | 208.492 | −16.568 | 1.00 | 50.00 | AAAA | C |
| ATOM | 206 | CD | GLU | A | 25 | 77.713 | 209.247 | −17.212 | 1.00 | 50.00 | AAAA | C |
| ATOM | 207 | OE1 | GLU | A | 25 | 78.232 | 208.813 | −18.239 | 1.00 | 50.00 | AAAA | O |
| ATOM | 208 | OE2 | GLU | A | 25 | 78.108 | 210.269 | −16.655 | 1.00 | 50.00 | AAAA | O |
| ATOM | 209 | H | GLU | A | 25 | 73.955 | 208.259 | −17.000 | 0.00 | 0.00 | AAAA | H |
| ATOM | 210 | N | GLY | A | 26 | 73.729 | 205.951 | −19.147 | 1.00 | 50.00 | AAAA | N |
| ATOM | 211 | CA | GLY | A | 26 | 73.117 | 204.987 | −20.054 | 1.00 | 50.00 | AAAA | C |
| ATOM | 212 | C | GLY | A | 26 | 72.711 | 205.603 | −21.378 | 1.00 | 50.00 | AAAA | C |
| ATOM | 213 | O | GLY | A | 26 | 72.962 | 205.043 | −22.435 | 1.00 | 50.00 | AAAA | O |
| ATOM | 214 | H | GLY | A | 26 | 73.227 | 206.304 | −18.358 | 0.00 | 0.00 | AAAA | H |
| ATOM | 215 | N | GLU | A | 27 | 72.092 | 206.797 | −21.270 | 1.00 | 50.00 | AAAA | N |
| ATOM | 216 | CA | GLU | A | 27 | 71.639 | 207.494 | −22.477 | 1.00 | 50.00 | AAAA | C |
| ATOM | 217 | C | GLU | A | 27 | 72.762 | 207.882 | −23.446 | 1.00 | 50.00 | AAAA | C |
| ATOM | 218 | O | GLU | A | 27 | 72.596 | 207.889 | −24.658 | 1.00 | 50.00 | AAAA | O |
| ATOM | 219 | CB | GLU | A | 27 | 70.751 | 208.693 | −22.086 | 1.00 | 50.00 | AAAA | C |
| ATOM | 220 | CG | GLU | A | 27 | 71.572 | 209.933 | −21.718 | 1.00 | 50.00 | AAAA | C |
| ATOM | 221 | CD | GLU | A | 27 | 70.850 | 210.987 | −20.913 | 1.00 | 50.00 | AAAA | C |
| ATOM | 222 | OE1 | GLU | A | 27 | 69.841 | 210.691 | −20.280 | 1.00 | 50.00 | AAAA | O |
| ATOM | 223 | OE2 | GLU | A | 27 | 71.331 | 212.117 | −20.914 | 1.00 | 50.00 | AAAA | O |
| ATOM | 224 | H | GLU | A | 27 | 71.907 | 207.187 | −20.368 | 0.00 | 0.00 | AAAA | H |
| ATOM | 225 | N | VAL | A | 28 | 73.931 | 208.187 | −22.836 | 1.00 | 50.00 | AAAA | N |
| ATOM | 226 | CA | VAL | A | 28 | 75.083 | 208.586 | −23.645 | 1.00 | 50.00 | AAAA | C |
| ATOM | 227 | C | VAL | A | 28 | 75.806 | 207.427 | −24.303 | 1.00 | 50.00 | AAAA | C |
| ATOM | 228 | O | VAL | A | 28 | 76.293 | 207.508 | −25.419 | 1.00 | 50.00 | AAAA | O |
| ATOM | 229 | CB | VAL | A | 28 | 76.072 | 209.477 | −22.865 | 1.00 | 50.00 | AAAA | C |
| ATOM | 230 | CG1 | VAL | A | 28 | 75.386 | 210.762 | −22.408 | 1.00 | 50.00 | AAAA | C |
| ATOM | 231 | CG2 | VAL | A | 28 | 76.793 | 208.777 | −21.711 | 1.00 | 50.00 | AAAA | C |
| ATOM | 232 | H | VAL | A | 28 | 74.010 | 208.150 | −21.840 | 0.00 | 0.00 | AAAA | H |
| ATOM | 233 | N | ASN | A | 29 | 75.815 | 206.317 | −23.553 | 1.00 | 50.00 | AAAA | N |
| ATOM | 234 | CA | ASN | A | 29 | 76.386 | 205.103 | −24.107 | 1.00 | 50.00 | AAAA | C |
| ATOM | 235 | C | ASN | A | 29 | 75.489 | 204.440 | −25.136 | 1.00 | 50.00 | AAAA | C |
| ATOM | 236 | O | ASN | A | 29 | 75.925 | 203.697 | −26.000 | 1.00 | 50.00 | AAAA | O |
| ATOM | 237 | CB | ASN | A | 29 | 76.666 | 204.143 | −22.974 | 1.00 | 50.00 | AAAA | C |
| ATOM | 238 | CG | ASN | A | 29 | 77.546 | 203.068 | −23.526 | 1.00 | 50.00 | AAAA | C |
| ATOM | 239 | ND2 | ASN | A | 29 | 77.056 | 201.834 | −23.370 | 1.00 | 50.00 | AAAA | N |
| ATOM | 240 | OD1 | ASN | A | 29 | 78.584 | 203.352 | −24.093 | 1.00 | 50.00 | AAAA | O |
| ATOM | 241 | H | ASN | A | 29 | 75.417 | 206.335 | −22.637 | 0.00 | 0.00 | AAAA | H |
| ATOM | 242 | 1HD2 | ASN | A | 29 | 77.519 | 201.060 | −23.798 | 0.00 | 0.00 | AAAA | H |
| ATOM | 243 | 2HD2 | ASN | A | 29 | 76.234 | 201.670 | −22.826 | 0.00 | 0.00 | AAAA | H |
| ATOM | 244 | N | LYS | A | 30 | 74.197 | 204.766 | −24.998 | 1.00 | 50.00 | AAAA | N |
| ATOM | 245 | CA | LYS | A | 30 | 73.226 | 204.271 | −25.964 | 1.00 | 50.00 | AAAA | C |
| ATOM | 246 | C | LYS | A | 30 | 73.514 | 204.697 | −27.399 | 1.00 | 50.00 | AAAA | C |
| ATOM | 247 | O | LYS | A | 30 | 73.187 | 204.017 | −28.365 | 1.00 | 50.00 | AAAA | O |
| ATOM | 248 | CB | LYS | A | 30 | 71.832 | 204.707 | −25.504 | 1.00 | 50.00 | AAAA | C |
| ATOM | 249 | CG | LYS | A | 30 | 70.672 | 204.032 | −26.230 | 1.00 | 50.00 | AAAA | C |
| ATOM | 250 | CD | LYS | A | 30 | 70.672 | 202.514 | −26.048 | 1.00 | 50.00 | AAAA | C |
| ATOM | 251 | CE | LYS | A | 30 | 69.550 | 201.838 | −26.837 | 1.00 | 50.00 | AAAA | C |
| ATOM | 252 | NZ | LYS | A | 30 | 69.750 | 202.066 | −28.276 | 1.00 | 50.00 | AAAA | N |
| ATOM | 253 | H | LYS | A | 30 | 73.900 | 205.354 | −24.246 | 0.00 | 0.00 | AAAA | H |
| ATOM | 254 | 1HZ | LYS | A | 30 | 68.978 | 201.621 | −28.812 | 0.00 | 0.00 | AAAA | H |
| ATOM | 255 | 2HZ | LYS | A | 30 | 70.658 | 201.649 | −28.568 | 0.00 | 0.00 | AAAA | H |
| ATOM | 256 | 3HZ | LYS | A | 30 | 69.764 | 203.088 | −28.470 | 0.00 | 0.00 | AAAA | H |
| ATOM | 257 | N | ILE | A | 31 | 74.182 | 205.865 | −27.479 | 1.00 | 50.00 | AAAA | N |
| ATOM | 258 | CA | ILE | A | 31 | 74.542 | 206.380 | −28.792 | 1.00 | 50.00 | AAAA | C |
| ATOM | 259 | C | ILE | A | 31 | 76.007 | 206.170 | −29.145 | 1.00 | 50.00 | AAAA | C |
| ATOM | 260 | O | ILE | A | 31 | 76.576 | 206.886 | −29.955 | 1.00 | 50.00 | AAAA | O |
| ATOM | 261 | CB | ILE | A | 31 | 74.130 | 207.858 | −28.911 | 1.00 | 50.00 | AAAA | C |
| ATOM | 262 | CG1 | ILE | A | 31 | 74.857 | 208.739 | −27.896 | 1.00 | 50.00 | AAAA | C |
| ATOM | 263 | CG2 | ILE | A | 31 | 72.616 | 207.981 | −28.712 | 1.00 | 50.00 | AAAA | C |
| ATOM | 264 | CD1 | ILE | A | 31 | 74.504 | 210.219 | −28.015 | 1.00 | 50.00 | AAAA | C |
| ATOM | 265 | H | ILE | A | 31 | 74.536 | 206.294 | −26.646 | 0.00 | 0.00 | AAAA | H |
| ATOM | 266 | N | LYS | A | 32 | 76.593 | 205.128 | −28.516 | 1.00 | 50.00 | AAAA | N |
| ATOM | 267 | CA | LYS | A | 32 | 77.999 | 204.781 | −28.739 | 1.00 | 50.00 | AAAA | C |
| ATOM | 268 | C | LYS | A | 32 | 78.402 | 204.672 | −30.201 | 1.00 | 50.00 | AAAA | C |
| ATOM | 269 | O | LYS | A | 32 | 79.478 | 205.085 | −30.603 | 1.00 | 50.00 | AAAA | O |
| ATOM | 270 | CB | LYS | A | 32 | 78.327 | 203.480 | −28.008 | 1.00 | 50.00 | AAAA | C |
| ATOM | 271 | CG | LYS | A | 32 | 77.515 | 202.276 | −28.508 | 1.00 | 50.00 | AAAA | C |
| ATOM | 272 | CD | LYS | A | 32 | 77.843 | 200.937 | −27.865 | 1.00 | 50.00 | AAAA | C |
| ATOM | 273 | CE | LYS | A | 32 | 77.243 | 200.735 | −26.483 | 1.00 | 50.00 | AAAA | C |
| ATOM | 274 | NZ | LYS | A | 32 | 75.793 | 200.612 | −26.641 | 1.00 | 50.00 | AAAA | N |
| ATOM | 275 | H | LYS | A | 32 | 76.070 | 204.600 | −27.847 | 0.00 | 0.00 | AAAA | H |
| ATOM | 276 | 1HZ | LYS | A | 32 | 75.345 | 200.549 | −25.705 | 0.00 | 0.00 | AAAA | H |
| ATOM | 277 | 2HZ | LYS | A | 32 | 75.600 | 199.748 | −27.189 | 0.00 | 0.00 | AAAA | H |
| ATOM | 278 | 3HZ | LYS | A | 32 | 75.425 | 201.440 | −27.154 | 0.00 | 0.00 | AAAA | H |
| ATOM | 279 | N | SER | A | 33 | 77.443 | 204.124 | −30.971 | 1.00 | 50.00 | AAAA | N |
| ATOM | 280 | CA | SER | A | 33 | 77.654 | 203.878 | −32.388 | 1.00 | 50.00 | AAAA | C |
| ATOM | 281 | C | SER | A | 33 | 77.762 | 205.158 | −33.191 | 1.00 | 50.00 | AAAA | C |
| ATOM | 282 | O | SER | A | 33 | 78.676 | 205.343 | −33.978 | 1.00 | 50.00 | AAAA | O |
| ATOM | 283 | CB | SER | A | 33 | 76.516 | 202.991 | −32.895 | 1.00 | 50.00 | AAAA | C |
| ATOM | 284 | OG | SER | A | 33 | 76.726 | 202.639 | −34.263 | 1.00 | 50.00 | AAAA | O |
| ATOM | 285 | H | SER | A | 33 | 76.588 | 203.846 | −30.538 | 0.00 | 0.00 | AAAA | H |
| ATOM | 286 | HG | SER | A | 33 | 75.927 | 202.218 | −34.558 | 0.00 | 0.00 | AAAA | H |
| ATOM | 287 | N | ALA | A | 34 | 76.782 | 206.047 | −32.937 | 1.00 | 50.00 | AAAA | N |
| ATOM | 288 | CA | ALA | A | 34 | 76.799 | 207.336 | −33.630 | 1.00 | 50.00 | AAAA | C |
| ATOM | 289 | C | ALA | A | 34 | 77.992 | 208.203 | −33.269 | 1.00 | 50.00 | AAAA | C |
| ATOM | 290 | O | ALA | A | 34 | 78.618 | 208.845 | −34.099 | 1.00 | 50.00 | AAAA | O |
| ATOM | 291 | CB | ALA | A | 34 | 75.518 | 208.113 | −33.321 | 1.00 | 50.00 | AAAA | C |
| ATOM | 292 | H | ALA | A | 34 | 76.097 | 205.858 | −32.234 | 0.00 | 0.00 | AAAA | H |
| ATOM | 293 | N | LEU | A | 35 | 78.280 | 208.156 | −31.960 | 1.00 | 50.00 | AAAA | N |
| ATOM | 294 | CA | LEU | A | 35 | 79.445 | 208.835 | −31.401 | 1.00 | 50.00 | AAAA | C |
| ATOM | 295 | C | LEU | A | 35 | 80.756 | 208.424 | −32.015 | 1.00 | 50.00 | AAAA | C |
| ATOM | 296 | O | LEU | A | 35 | 81.664 | 209.215 | −32.233 | 1.00 | 50.00 | AAAA | O |
| ATOM | 297 | CB | LEU | A | 35 | 79.520 | 208.547 | −29.912 | 1.00 | 50.00 | AAAA | C |
| ATOM | 298 | CG | LEU | A | 35 | 78.397 | 209.185 | −29.116 | 1.00 | 50.00 | AAAA | C |
| ATOM | 299 | CD1 | LEU | A | 35 | 78.563 | 208.916 | −27.620 | 1.00 | 50.00 | AAAA | C |
| ATOM | 300 | CD2 | LEU | A | 35 | 78.225 | 210.656 | −29.486 | 1.00 | 50.00 | AAAA | C |
| ATOM | 301 | H | LEU | A | 35 | 77.693 | 207.603 | −31.375 | 0.00 | 0.00 | AAAA | H |
| ATOM | 302 | N | LEU | A | 36 | 80.794 | 207.110 | −32.268 | 1.00 | 50.00 | AAAA | N |
| ATOM | 303 | CA | LEU | A | 36 | 82.009 | 206.541 | −32.818 | 1.00 | 50.00 | AAAA | C |
| ATOM | 304 | C | LEU | A | 36 | 82.172 | 206.820 | −34.301 | 1.00 | 50.00 | AAAA | C |
| ATOM | 305 | O | LEU | A | 36 | 83.255 | 207.117 | −34.791 | 1.00 | 50.00 | AAAA | O |
| ATOM | 306 | CB | LEU | A | 36 | 82.058 | 205.068 | −32.401 | 1.00 | 50.00 | AAAA | C |
| ATOM | 307 | CG | LEU | A | 36 | 83.409 | 204.390 | −32.586 | 1.00 | 50.00 | AAAA | C |
| ATOM | 308 | CD1 | LEU | A | 36 | 83.588 | 203.163 | −31.699 | 1.00 | 50.00 | AAAA | C |
| ATOM | 309 | CD2 | LEU | A | 36 | 83.596 | 203.995 | −34.034 | 1.00 | 50.00 | AAAA | C |
| ATOM | 310 | H | LEU | A | 36 | 79.988 | 206.542 | −32.103 | 0.00 | 0.00 | AAAA | H |
| ATOM | 311 | N | SER | A | 37 | 81.015 | 206.733 | −34.981 | 1.00 | 50.00 | AAAA | N |
| ATOM | 312 | CA | SER | A | 37 | 81.007 | 206.968 | −36.424 | 1.00 | 50.00 | AAAA | C |
| ATOM | 313 | C | SER | A | 37 | 81.384 | 208.384 | −36.819 | 1.00 | 50.00 | AAAA | C |
| ATOM | 314 | O | SER | A | 37 | 82.024 | 208.625 | −37.838 | 1.00 | 50.00 | AAAA | O |
| ATOM | 315 | CB | SER | A | 37 | 79.642 | 206.606 | −37.016 | 1.00 | 50.00 | AAAA | C |
| ATOM | 316 | OG | SER | A | 37 | 79.323 | 205.239 | −36.730 | 1.00 | 50.00 | AAAA | O |
| ATOM | 317 | H | SER | A | 37 | 80.159 | 206.522 | −34.514 | 0.00 | 0.00 | AAAA | H |
| ATOM | 318 | HG | SER | A | 37 | 78.511 | 205.054 | −37.182 | 0.00 | 0.00 | AAAA | H |
| ATOM | 319 | N | THR | A | 38 | 80.963 | 209.311 | −35.936 | 1.00 | 50.00 | AAAA | N |
| ATOM | 320 | CA | THR | A | 38 | 81.295 | 210.718 | −36.124 | 1.00 | 50.00 | AAAA | C |
| ATOM | 321 | C | THR | A | 38 | 82.778 | 211.028 | −35.957 | 1.00 | 50.00 | AAAA | C |
| ATOM | 322 | O | THR | A | 38 | 83.409 | 210.730 | −34.949 | 1.00 | 50.00 | AAAA | O |
| ATOM | 323 | CB | THR | A | 38 | 80.436 | 211.570 | −35.180 | 1.00 | 50.00 | AAAA | C |
| ATOM | 324 | CG2 | THR | A | 38 | 80.642 | 213.076 | −35.369 | 1.00 | 50.00 | AAAA | C |
| ATOM | 325 | OG1 | THR | A | 38 | 79.053 | 211.242 | −35.354 | 1.00 | 50.00 | AAAA | O |
| ATOM | 326 | H | THR | A | 38 | 80.459 | 209.024 | −35.122 | 0.00 | 0.00 | AAAA | H |
| ATOM | 327 | HG1 | THR | A | 38 | 78.569 | 211.813 | −34.776 | 0.00 | 0.00 | AAAA | H |
| ATOM | 328 | N | ASN | A | 39 | 83.296 | 211.660 | −37.025 | 1.00 | 50.00 | AAAA | N |
| ATOM | 329 | CA | ASN | A | 39 | 84.661 | 212.180 | −36.973 | 1.00 | 50.00 | AAAA | C |
| ATOM | 330 | C | ASN | A | 39 | 84.634 | 213.656 | −36.602 | 1.00 | 50.00 | AAAA | C |
| ATOM | 331 | O | ASN | A | 39 | 84.251 | 214.497 | −37.405 | 1.00 | 50.00 | AAAA | O |
| ATOM | 332 | CB | ASN | A | 39 | 85.356 | 211.956 | −38.326 | 1.00 | 50.00 | AAAA | C |
| ATOM | 333 | CG | ASN | A | 39 | 86.806 | 212.419 | −38.319 | 1.00 | 50.00 | AAAA | C |
| ATOM | 334 | ND2 | ASN | A | 39 | 87.594 | 211.686 | −39.116 | 1.00 | 50.00 | AAAA | N |
| ATOM | 335 | OD1 | ASN | A | 39 | 87.204 | 213.378 | −37.669 | 1.00 | 50.00 | AAAA | O |
| ATOM | 336 | H | ASN | A | 39 | 82.726 | 211.852 | −37.823 | 0.00 | 0.00 | AAAA | H |
| ATOM | 337 | 1HD2 | ASN | A | 39 | 88.555 | 211.942 | −39.211 | 0.00 | 0.00 | AAAA | H |
| ATOM | 338 | 2HD2 | ASN | A | 39 | 87.249 | 210.903 | −39.631 | 0.00 | 0.00 | AAAA | H |
| ATOM | 339 | N | LYS | A | 40 | 85.063 | 213.911 | −35.350 | 1.00 | 50.00 | AAAA | N |
| ATOM | 340 | CA | LYS | A | 40 | 85.052 | 215.262 | −34.782 | 1.00 | 50.00 | AAAA | C |
| ATOM | 341 | C | LYS | A | 40 | 85.641 | 215.359 | −33.375 | 1.00 | 50.00 | AAAA | C |
| ATOM | 342 | O | LYS | A | 40 | 85.124 | 214.763 | −32.439 | 1.00 | 50.00 | AAAA | O |
| ATOM | 343 | CB | LYS | A | 40 | 83.621 | 215.802 | −34.739 | 1.00 | 50.00 | AAAA | C |
| ATOM | 344 | CG | LYS | A | 40 | 83.538 | 217.259 | −34.304 | 1.00 | 50.00 | AAAA | C |
| ATOM | 345 | CD | LYS | A | 40 | 84.367 | 218.205 | −35.169 | 1.00 | 50.00 | AAAA | C |
| ATOM | 346 | CE | LYS | A | 40 | 84.389 | 219.626 | −34.608 | 1.00 | 50.00 | AAAA | C |
| ATOM | 347 | NZ | LYS | A | 40 | 85.147 | 219.670 | −33.349 | 1.00 | 50.00 | AAAA | N |
| ATOM | 348 | H | LYS | A | 40 | 85.379 | 213.149 | −34.796 | 0.00 | 0.00 | AAAA | H |
| ATOM | 349 | 1HZ | LYS | A | 40 | 85.073 | 220.617 | −32.926 | 0.00 | 0.00 | AAAA | H |
| ATOM | 350 | 2HZ | LYS | A | 40 | 86.146 | 219.467 | −33.550 | 0.00 | 0.00 | AAAA | H |
| ATOM | 351 | 3HZ | LYS | A | 40 | 84.793 | 218.962 | −32.672 | 0.00 | 0.00 | AAAA | H |
| ATOM | 352 | N | ALA | A | 41 | 86.720 | 216.170 | −33.234 | 1.00 | 50.00 | AAAA | N |
| ATOM | 353 | CA | ALA | A | 41 | 87.393 | 216.261 | −31.927 | 1.00 | 50.00 | AAAA | C |
| ATOM | 354 | C | ALA | A | 41 | 86.514 | 216.629 | −30.737 | 1.00 | 50.00 | AAAA | C |
| ATOM | 355 | O | ALA | A | 41 | 86.722 | 216.178 | −29.616 | 1.00 | 50.00 | AAAA | O |
| ATOM | 356 | CB | ALA | A | 41 | 88.545 | 217.264 | −31.972 | 1.00 | 50.00 | AAAA | C |
| ATOM | 357 | H | ALA | A | 41 | 87.105 | 216.640 | −34.030 | 0.00 | 0.00 | AAAA | H |
| ATOM | 358 | N | VAL | A | 42 | 85.516 | 217.481 | −31.055 | 1.00 | 50.00 | AAAA | N |
| ATOM | 359 | CA | VAL | A | 42 | 84.484 | 217.844 | −30.077 | 1.00 | 50.00 | AAAA | C |
| ATOM | 360 | C | VAL | A | 42 | 83.115 | 217.859 | −30.745 | 1.00 | 50.00 | AAAA | C |
| ATOM | 361 | O | VAL | A | 42 | 82.896 | 218.650 | −31.652 | 1.00 | 50.00 | AAAA | O |
| ATOM | 362 | CB | VAL | A | 42 | 84.717 | 219.251 | −29.493 | 1.00 | 50.00 | AAAA | C |
| ATOM | 363 | CG1 | VAL | A | 42 | 83.785 | 219.530 | −28.312 | 1.00 | 50.00 | AAAA | C |
| ATOM | 364 | CG2 | VAL | A | 42 | 86.176 | 219.556 | −29.168 | 1.00 | 50.00 | AAAA | C |
| ATOM | 365 | H | VAL | A | 42 | 85.481 | 217.842 | −31.986 | 0.00 | 0.00 | AAAA | H |
| ATOM | 366 | N | VAL | A | 43 | 82.202 | 216.997 | −30.267 | 1.00 | 50.00 | AAAA | N |
| ATOM | 367 | CA | VAL | A | 43 | 80.872 | 216.989 | −30.883 | 1.00 | 50.00 | AAAA | C |
| ATOM | 368 | C | VAL | A | 43 | 79.761 | 216.857 | −29.846 | 1.00 | 50.00 | AAAA | C |
| ATOM | 369 | O | VAL | A | 43 | 79.908 | 216.215 | −28.817 | 1.00 | 50.00 | AAAA | O |
| ATOM | 370 | CB | VAL | A | 43 | 80.807 | 215.923 | −32.006 | 1.00 | 50.00 | AAAA | C |
| ATOM | 371 | CG1 | VAL | A | 43 | 81.221 | 214.531 | −31.543 | 1.00 | 50.00 | AAAA | C |
| ATOM | 372 | CG2 | VAL | A | 43 | 79.476 | 215.892 | −32.762 | 1.00 | 50.00 | AAAA | C |
| ATOM | 373 | H | VAL | A | 43 | 82.405 | 216.390 | −29.499 | 0.00 | 0.00 | AAAA | H |
| ATOM | 374 | N | SER | A | 44 | 78.637 | 217.529 | −30.151 | 1.00 | 50.00 | AAAA | N |
| ATOM | 375 | CA | SER | A | 44 | 77.490 | 217.433 | −29.253 | 1.00 | 50.00 | AAAA | C |
| ATOM | 376 | C | SER | A | 44 | 76.730 | 216.120 | −29.430 | 1.00 | 50.00 | AAAA | C |
| ATOM | 377 | O | SER | A | 44 | 76.397 | 215.697 | −30.530 | 1.00 | 50.00 | AAAA | O |
| ATOM | 378 | CB | SER | A | 44 | 76.590 | 218.655 | −29.482 | 1.00 | 50.00 | AAAA | C |
| ATOM | 379 | OG | SER | A | 44 | 75.460 | 218.633 | −28.606 | 1.00 | 50.00 | AAAA | O |
| ATOM | 380 | H | SER | A | 44 | 78.548 | 217.996 | −31.029 | 0.00 | 0.00 | AAAA | H |
| ATOM | 381 | HG | SER | A | 44 | 74.946 | 219.417 | −28.768 | 0.00 | 0.00 | AAAA | H |
| ATOM | 382 | N | LEU | A | 45 | 76.479 | 215.495 | −28.272 | 1.00 | 50.00 | AAAA | N |
| ATOM | 383 | CA | LEU | A | 45 | 75.666 | 214.285 | −28.180 | 1.00 | 50.00 | AAAA | C |
| ATOM | 384 | C | LEU | A | 45 | 74.217 | 214.678 | −28.139 | 1.00 | 50.00 | AAAA | C |
| ATOM | 385 | O | LEU | A | 45 | 73.782 | 215.388 | −27.244 | 1.00 | 50.00 | AAAA | O |
| ATOM | 386 | CB | LEU | A | 45 | 75.943 | 213.500 | −26.891 | 1.00 | 50.00 | AAAA | C |
| ATOM | 387 | CG | LEU | A | 45 | 77.213 | 212.660 | −26.854 | 1.00 | 50.00 | AAAA | C |
| ATOM | 388 | CD1 | LEU | A | 45 | 78.444 | 213.510 | −27.005 | 1.00 | 50.00 | AAAA | C |
| ATOM | 389 | CD2 | LEU | A | 45 | 77.332 | 211.823 | −25.588 | 1.00 | 50.00 | AAAA | C |
| ATOM | 390 | H | LEU | A | 45 | 76.786 | 215.944 | −27.439 | 0.00 | 0.00 | AAAA | H |
| ATOM | 391 | N | SER | A | 46 | 73.489 | 214.203 | −29.154 | 1.00 | 50.00 | AAAA | N |
| ATOM | 392 | CA | SER | A | 46 | 72.094 | 214.616 | −29.185 | 1.00 | 50.00 | AAAA | C |
| ATOM | 393 | C | SER | A | 46 | 71.118 | 213.459 | −29.272 | 1.00 | 50.00 | AAAA | C |
| ATOM | 394 | O | SER | A | 46 | 71.324 | 212.474 | −29.970 | 1.00 | 50.00 | AAAA | O |
| ATOM | 395 | CB | SER | A | 46 | 71.876 | 215.639 | −30.309 | 1.00 | 50.00 | AAAA | C |
| ATOM | 396 | OG | SER | A | 46 | 70.535 | 216.152 | −30.300 | 1.00 | 50.00 | AAAA | O |
| ATOM | 397 | H | SER | A | 46 | 73.887 | 213.639 | −29.877 | 0.00 | 0.00 | AAAA | H |
| ATOM | 398 | HG | SER | A | 46 | 70.534 | 216.892 | −30.893 | 0.00 | 0.00 | AAAA | H |
| ATOM | 399 | N | ASN | A | 47 | 70.025 | 213.664 | −28.519 | 1.00 | 50.00 | AAAA | N |
| ATOM | 400 | CA | ASN | A | 47 | 68.899 | 212.739 | −28.567 | 1.00 | 50.00 | AAAA | C |
| ATOM | 401 | C | ASN | A | 47 | 67.624 | 213.532 | −28.396 | 1.00 | 50.00 | AAAA | C |
| ATOM | 402 | O | ASN | A | 47 | 67.429 | 214.197 | −27.387 | 1.00 | 50.00 | AAAA | O |
| ATOM | 403 | CB | ASN | A | 47 | 69.030 | 211.680 | −27.466 | 1.00 | 50.00 | AAAA | C |
| ATOM | 404 | CG | ASN | A | 47 | 67.928 | 210.644 | −27.563 | 1.00 | 50.00 | AAAA | C |
| ATOM | 405 | ND2 | ASN | A | 47 | 67.179 | 210.549 | −26.456 | 1.00 | 50.00 | AAAA | N |
| ATOM | 406 | OD1 | ASN | A | 47 | 67.755 | 209.973 | −28.571 | 1.00 | 50.00 | AAAA | O |
| ATOM | 407 | H | ASN | A | 47 | 69.980 | 214.488 | −27.952 | 0.00 | 0.00 | AAAA | H |
| ATOM | 408 | 1HD2 | ASN | A | 47 | 66.420 | 209.899 | −26.433 | 0.00 | 0.00 | AAAA | H |
| ATOM | 409 | 2HD2 | ASN | A | 47 | 67.345 | 211.117 | −25.649 | 0.00 | 0.00 | AAAA | H |
| ATOM | 410 | N | GLY | A | 48 | 66.780 | 213.433 | −29.439 | 1.00 | 50.00 | AAAA | N |
| ATOM | 411 | CA | GLY | A | 48 | 65.487 | 214.123 | −29.416 | 1.00 | 50.00 | AAAA | C |
| ATOM | 412 | C | GLY | A | 48 | 65.548 | 215.628 | −29.207 | 1.00 | 50.00 | AAAA | C |
| ATOM | 413 | O | GLY | A | 48 | 64.804 | 216.197 | −28.419 | 1.00 | 50.00 | AAAA | O |
| ATOM | 414 | H | GLY | A | 48 | 67.027 | 212.816 | −30.186 | 0.00 | 0.00 | AAAA | H |
| ATOM | 415 | N | VAL | A | 49 | 66.488 | 216.241 | −29.963 | 1.00 | 50.00 | AAAA | N |
| ATOM | 416 | CA | VAL | A | 49 | 66.736 | 217.688 | −29.863 | 1.00 | 50.00 | AAAA | C |
| ATOM | 417 | C | VAL | A | 49 | 67.160 | 218.144 | −28.461 | 1.00 | 50.00 | AAAA | C |
| ATOM | 418 | O | VAL | A | 49 | 66.759 | 219.172 | −27.930 | 1.00 | 50.00 | AAAA | O |
| ATOM | 419 | CB | VAL | A | 49 | 65.552 | 218.491 | −30.467 | 1.00 | 50.00 | AAAA | C |
| ATOM | 420 | CG1 | VAL | A | 49 | 65.807 | 219.997 | −30.621 | 1.00 | 50.00 | AAAA | C |
| ATOM | 421 | CG2 | VAL | A | 49 | 65.190 | 217.919 | −31.843 | 1.00 | 50.00 | AAAA | C |
| ATOM | 422 | H | VAL | A | 49 | 67.020 | 215.691 | −30.604 | 0.00 | 0.00 | AAAA | H |
| ATOM | 423 | N | SER | A | 50 | 68.025 | 217.288 | −27.884 | 1.00 | 50.00 | AAAA | N |
| ATOM | 424 | CA | SER | A | 50 | 68.574 | 217.628 | −26.576 | 1.00 | 50.00 | AAAA | C |
| ATOM | 425 | C | SER | A | 50 | 70.042 | 217.277 | −26.502 | 1.00 | 50.00 | AAAA | C |
| ATOM | 426 | O | SER | A | 50 | 70.506 | 216.313 | −27.100 | 1.00 | 50.00 | AAAA | O |
| ATOM | 427 | CB | SER | A | 50 | 67.810 | 216.914 | −25.456 | 1.00 | 50.00 | AAAA | C |
| ATOM | 428 | OG | SER | A | 50 | 66.430 | 217.288 | −25.498 | 1.00 | 50.00 | AAAA | O |
| ATOM | 429 | H | SER | A | 50 | 68.245 | 216.415 | −28.316 | 0.00 | 0.00 | AAAA | H |
| ATOM | 430 | HG | SER | A | 50 | 65.987 | 216.807 | −24.812 | 0.00 | 0.00 | AAAA | H |
| ATOM | 431 | N | VAL | A | 51 | 70.767 | 218.114 | −25.742 | 1.00 | 50.00 | AAAA | N |
| ATOM | 432 | CA | VAL | A | 51 | 72.175 | 217.784 | −25.576 | 1.00 | 50.00 | AAAA | C |
| ATOM | 433 | C | VAL | A | 51 | 72.385 | 216.961 | −24.324 | 1.00 | 50.00 | AAAA | C |
| ATOM | 434 | O | VAL | A | 51 | 72.218 | 217.391 | −23.189 | 1.00 | 50.00 | AAAA | O |
| ATOM | 435 | CB | VAL | A | 51 | 73.076 | 219.033 | −25.648 | 1.00 | 50.00 | AAAA | C |
| ATOM | 436 | CG1 | VAL | A | 51 | 72.667 | 220.133 | −24.664 | 1.00 | 50.00 | AAAA | C |
| ATOM | 437 | CG2 | VAL | A | 51 | 74.559 | 218.669 | −25.542 | 1.00 | 50.00 | AAAA | C |
| ATOM | 438 | H | VAL | A | 51 | 70.333 | 218.825 | −25.189 | 0.00 | 0.00 | AAAA | H |
| ATOM | 439 | N | LEU | A | 52 | 72.739 | 215.703 | −24.612 | 1.00 | 50.00 | AAAA | N |
| ATOM | 440 | CA | LEU | A | 52 | 73.023 | 214.816 | −23.501 | 1.00 | 50.00 | AAAA | C |
| ATOM | 441 | C | LEU | A | 52 | 74.372 | 215.080 | −22.912 | 1.00 | 50.00 | AAAA | C |
| ATOM | 442 | O | LEU | A | 52 | 74.520 | 214.827 | −21.725 | 1.00 | 50.00 | AAAA | O |
| ATOM | 443 | CB | LEU | A | 52 | 72.943 | 213.355 | −23.907 | 1.00 | 50.00 | AAAA | C |
| ATOM | 444 | CG | LEU | A | 52 | 71.621 | 213.000 | −24.572 | 1.00 | 50.00 | AAAA | C |
| ATOM | 445 | CD1 | LEU | A | 52 | 71.618 | 211.522 | −24.950 | 1.00 | 50.00 | AAAA | C |
| ATOM | 446 | CD2 | LEU | A | 52 | 70.392 | 213.401 | −23.749 | 1.00 | 50.00 | AAAA | C |
| ATOM | 447 | H | LEU | A | 52 | 72.906 | 215.401 | −25.547 | 0.00 | 0.00 | AAAA | H |
| ATOM | 448 | N | THR | A | 53 | 75.290 | 215.588 | −23.814 | 1.00 | 50.00 | AAAA | N |
| ATOM | 449 | CA | THR | A | 53 | 76.668 | 216.062 | −23.552 | 1.00 | 50.00 | AAAA | C |
| ATOM | 450 | C | THR | A | 53 | 77.683 | 216.328 | −24.670 | 1.00 | 50.00 | AAAA | C |
| ATOM | 451 | O | THR | A | 53 | 77.305 | 216.587 | −25.798 | 1.00 | 50.00 | AAAA | O |
| ATOM | 452 | CB | THR | A | 53 | 77.330 | 215.322 | −22.434 | 1.00 | 50.00 | AAAA | C |
| ATOM | 453 | CG2 | THR | A | 53 | 76.956 | 216.243 | −21.277 | 1.00 | 50.00 | AAAA | C |
| ATOM | 454 | OG1 | THR | A | 53 | 76.919 | 213.948 | −22.288 | 1.00 | 50.00 | AAAA | O |
| ATOM | 455 | H | THR | A | 53 | 74.956 | 215.771 | −24.737 | 0.00 | 0.00 | AAAA | H |
| ATOM | 456 | HG1 | THR | A | 53 | 77.656 | 213.383 | −22.486 | 0.00 | 0.00 | AAAA | H |
| ATOM | 457 | N | SER | A | 54 | 79.003 | 216.286 | −24.326 | 1.00 | 50.00 | AAAA | N |
| ATOM | 458 | CA | SER | A | 54 | 80.047 | 216.438 | −25.344 | 1.00 | 50.00 | AAAA | C |
| ATOM | 459 | C | SER | A | 54 | 80.914 | 215.197 | −25.460 | 1.00 | 50.00 | AAAA | C |
| ATOM | 460 | O | SER | A | 54 | 81.180 | 214.502 | −24.490 | 1.00 | 50.00 | AAAA | O |
| ATOM | 461 | CB | SER | A | 54 | 80.902 | 217.676 | −25.053 | 1.00 | 50.00 | AAAA | C |
| ATOM | 462 | OG | SER | A | 54 | 82.001 | 217.803 | −25.963 | 1.00 | 50.00 | AAAA | O |
| ATOM | 463 | H | SER | A | 54 | 79.328 | 216.129 | −23.394 | 0.00 | 0.00 | AAAA | H |
| ATOM | 464 | HG | SER | A | 54 | 82.321 | 218.692 | −25.876 | 0.00 | 0.00 | AAAA | H |
| ATOM | 465 | N | LYS | A | 55 | 81.308 | 214.971 | −26.724 | 1.00 | 50.00 | AAAA | N |
| ATOM | 466 | CA | LYS | A | 55 | 82.095 | 213.829 | −27.169 | 1.00 | 50.00 | AAAA | C |
| ATOM | 467 | C | LYS | A | 55 | 83.449 | 214.314 | −27.578 | 1.00 | 50.00 | AAAA | C |
| ATOM | 468 | O | LYS | A | 55 | 83.605 | 215.179 | −28.430 | 1.00 | 50.00 | AAAA | O |
| ATOM | 469 | CB | LYS | A | 55 | 81.473 | 213.205 | −28.409 | 1.00 | 50.00 | AAAA | C |
| ATOM | 470 | CG | LYS | A | 55 | 82.092 | 211.905 | −28.886 | 1.00 | 50.00 | AAAA | C |
| ATOM | 471 | CD | LYS | A | 55 | 81.684 | 210.812 | −27.923 | 1.00 | 50.00 | AAAA | C |
| ATOM | 472 | CE | LYS | A | 55 | 82.425 | 209.526 | −28.146 | 1.00 | 50.00 | AAAA | C |
| ATOM | 473 | NZ | LYS | A | 55 | 83.834 | 209.862 | −28.034 | 1.00 | 50.00 | AAAA | N |
| ATOM | 474 | H | LYS | A | 55 | 81.066 | 215.667 | −27.394 | 0.00 | 0.00 | AAAA | H |
| ATOM | 475 | 1HZ | LYS | A | 55 | 84.334 | 208.954 | −28.099 | 0.00 | 0.00 | AAAA | H |
| ATOM | 476 | 2HZ | LYS | A | 55 | 84.064 | 210.314 | −27.127 | 0.00 | 0.00 | AAAA | H |
| ATOM | 477 | 3HZ | LYS | A | 55 | 84.123 | 210.470 | −28.827 | 0.00 | 0.00 | AAAA | H |
| ATOM | 478 | N | VAL | A | 56 | 84.443 | 213.734 | −26.910 | 1.00 | 50.00 | AAAA | N |
| ATOM | 479 | CA | VAL | A | 56 | 85.777 | 214.210 | −27.203 | 1.00 | 50.00 | AAAA | C |
| ATOM | 480 | C | VAL | A | 56 | 86.654 | 213.095 | −27.733 | 1.00 | 50.00 | AAAA | C |
| ATOM | 481 | O | VAL | A | 56 | 86.509 | 211.945 | −27.356 | 1.00 | 50.00 | AAAA | O |
| ATOM | 482 | CB | VAL | A | 56 | 86.337 | 214.900 | −25.945 | 1.00 | 50.00 | AAAA | C |
| ATOM | 483 | CG1 | VAL | A | 56 | 85.234 | 215.720 | −25.260 | 1.00 | 50.00 | AAAA | C |
| ATOM | 484 | CG2 | VAL | A | 56 | 86.995 | 213.972 | −24.929 | 1.00 | 50.00 | AAAA | C |
| ATOM | 485 | H | VAL | A | 56 | 84.303 | 213.028 | −26.220 | 0.00 | 0.00 | AAAA | H |
| ATOM | 486 | N | LEU | A | 57 | 87.580 | 213.480 | −28.619 | 1.00 | 50.00 | AAAA | N |
| ATOM | 487 | CA | LEU | A | 57 | 88.673 | 212.573 | −28.953 | 1.00 | 50.00 | AAAA | C |
| ATOM | 488 | C | LEU | A | 57 | 89.871 | 213.422 | −29.260 | 1.00 | 50.00 | AAAA | C |
| ATOM | 489 | O | LEU | A | 57 | 89.982 | 214.137 | −30.246 | 1.00 | 50.00 | AAAA | O |
| ATOM | 490 | CB | LEU | A | 57 | 88.340 | 211.565 | −30.068 | 1.00 | 50.00 | AAAA | C |
| ATOM | 491 | CG | LEU | A | 57 | 89.466 | 210.690 | −30.682 | 1.00 | 50.00 | AAAA | C |
| ATOM | 492 | CD1 | LEU | A | 57 | 90.407 | 210.050 | −29.668 | 1.00 | 50.00 | AAAA | C |
| ATOM | 493 | CD2 | LEU | A | 57 | 88.923 | 209.597 | −31.609 | 1.00 | 50.00 | AAAA | C |
| ATOM | 494 | H | LEU | A | 57 | 87.591 | 214.417 | −28.967 | 0.00 | 0.00 | AAAA | H |
| ATOM | 495 | N | ASP | A | 58 | 90.756 | 213.294 | −28.274 | 1.00 | 50.00 | AAAA | N |
| ATOM | 496 | CA | ASP | A | 58 | 92.020 | 214.011 | −28.150 | 1.00 | 50.00 | AAAA | C |
| ATOM | 497 | C | ASP | A | 58 | 92.924 | 214.039 | −29.370 | 1.00 | 50.00 | AAAA | C |
| ATOM | 498 | O | ASP | A | 58 | 93.619 | 215.012 | −29.634 | 1.00 | 50.00 | AAAA | O |
| ATOM | 499 | CB | ASP | A | 58 | 92.757 | 213.438 | −26.939 | 1.00 | 50.00 | AAAA | C |
| ATOM | 500 | CG | ASP | A | 58 | 92.743 | 211.921 | −26.938 | 1.00 | 50.00 | AAAA | C |
| ATOM | 501 | OD1 | ASP | A | 58 | 93.444 | 211.320 | −27.745 | 1.00 | 50.00 | AAAA | O |
| ATOM | 502 | OD2 | ASP | A | 58 | 92.019 | 211.349 | −26.127 | 1.00 | 50.00 | AAAA | O |
| ATOM | 503 | H | ASP | A | 58 | 90.504 | 212.666 | −27.535 | 0.00 | 0.00 | AAAA | H |
| ATOM | 504 | N | LEU | A | 59 | 92.902 | 212.903 | −30.079 | 1.00 | 50.00 | AAAA | N |
| ATOM | 505 | CA | LEU | A | 59 | 93.845 | 212.783 | −31.176 | 1.00 | 50.00 | AAAA | C |
| ATOM | 506 | C | LEU | A | 59 | 93.277 | 213.113 | −32.530 | 1.00 | 50.00 | AAAA | C |
| ATOM | 507 | O | LEU | A | 59 | 94.027 | 213.339 | −33.463 | 1.00 | 50.00 | AAAA | O |
| ATOM | 508 | CB | LEU | A | 59 | 94.452 | 211.390 | −31.202 | 1.00 | 50.00 | AAAA | C |
| ATOM | 509 | CG | LEU | A | 59 | 95.449 | 211.117 | −30.084 | 1.00 | 50.00 | AAAA | C |
| ATOM | 510 | CD1 | LEU | A | 59 | 95.953 | 209.687 | −30.240 | 1.00 | 50.00 | AAAA | C |
| ATOM | 511 | CD2 | LEU | A | 59 | 96.578 | 212.152 | −29.991 | 1.00 | 50.00 | AAAA | C |
| ATOM | 512 | H | LEU | A | 59 | 92.254 | 212.172 | −29.881 | 0.00 | 0.00 | AAAA | H |
| ATOM | 513 | N | LYS | A | 60 | 91.931 | 213.139 | −32.593 | 1.00 | 50.00 | AAAA | N |
| ATOM | 514 | CA | LYS | A | 60 | 91.223 | 213.333 | −33.859 | 1.00 | 50.00 | AAAA | C |
| ATOM | 515 | C | LYS | A | 60 | 91.776 | 214.359 | −34.827 | 1.00 | 50.00 | AAAA | C |
| ATOM | 516 | O | LYS | A | 60 | 91.971 | 214.064 | −35.996 | 1.00 | 50.00 | AAAA | O |
| ATOM | 517 | CB | LYS | A | 60 | 89.765 | 213.652 | −33.599 | 1.00 | 50.00 | AAAA | C |
| ATOM | 518 | CG | LYS | A | 60 | 88.840 | 212.485 | −33.909 | 1.00 | 50.00 | AAAA | C |
| ATOM | 519 | CD | LYS | A | 60 | 87.515 | 212.763 | −33.222 | 1.00 | 50.00 | AAAA | C |
| ATOM | 520 | CE | LYS | A | 60 | 86.464 | 211.661 | −33.271 | 1.00 | 50.00 | AAAA | C |
| ATOM | 521 | NZ | LYS | A | 60 | 85.336 | 212.046 | −32.398 | 1.00 | 50.00 | AAAA | N |
| ATOM | 522 | H | LYS | A | 60 | 91.395 | 212.987 | −31.766 | 0.00 | 0.00 | AAAA | H |
| ATOM | 523 | 1HZ | LYS | A | 60 | 84.778 | 211.204 | −32.151 | 0.00 | 0.00 | AAAA | H |
| ATOM | 524 | 2HZ | LYS | A | 60 | 84.726 | 212.742 | −32.872 | 0.00 | 0.00 | AAAA | H |
| ATOM | 525 | 3HZ | LYS | A | 60 | 85.704 | 212.463 | −31.516 | 0.00 | 0.00 | AAAA | H |
| ATOM | 526 | N | ASN | A | 61 | 92.030 | 215.562 | −34.277 | 1.00 | 50.00 | AAAA | N |
| ATOM | 527 | CA | ASN | A | 61 | 92.559 | 216.647 | −35.111 | 1.00 | 50.00 | AAAA | C |
| ATOM | 528 | C | ASN | A | 61 | 93.911 | 216.359 | −35.743 | 1.00 | 50.00 | AAAA | C |
| ATOM | 529 | O | ASN | A | 61 | 94.154 | 216.654 | −36.907 | 1.00 | 50.00 | AAAA | O |
| ATOM | 530 | CB | ASN | A | 61 | 92.619 | 217.964 | −34.331 | 1.00 | 50.00 | AAAA | C |
| ATOM | 531 | CG | ASN | A | 61 | 91.225 | 218.509 | −34.102 | 1.00 | 50.00 | AAAA | C |
| ATOM | 532 | ND2 | ASN | A | 61 | 91.138 | 219.329 | −33.046 | 1.00 | 50.00 | AAAA | N |
| ATOM | 533 | OD1 | ASN | A | 61 | 90.280 | 218.211 | −34.822 | 1.00 | 50.00 | AAAA | O |
| ATOM | 534 | H | ASN | A | 61 | 91.797 | 215.698 | −33.317 | 0.00 | 0.00 | AAAA | H |
| ATOM | 535 | 1HD2 | ASN | A | 61 | 90.259 | 219.718 | −32.776 | 0.00 | 0.00 | AAAA | H |
| ATOM | 536 | 2HD2 | ASN | A | 61 | 91.953 | 219.565 | −32.517 | 0.00 | 0.00 | AAAA | H |
| ATOM | 537 | N | TYR | A | 62 | 94.768 | 215.740 | −34.910 | 1.00 | 50.00 | AAAA | N |
| ATOM | 538 | CA | TYR | A | 62 | 96.084 | 215.305 | −35.374 | 1.00 | 50.00 | AAAA | C |
| ATOM | 539 | C | TYR | A | 62 | 96.063 | 214.119 | −36.351 | 1.00 | 50.00 | AAAA | C |
| ATOM | 540 | O | TYR | A | 62 | 96.779 | 214.134 | −37.344 | 1.00 | 50.00 | AAAA | O |
| ATOM | 541 | CB | TYR | A | 62 | 96.972 | 215.123 | −34.127 | 1.00 | 50.00 | AAAA | C |
| ATOM | 542 | CG | TYR | A | 62 | 98.424 | 214.808 | −34.438 | 1.00 | 50.00 | AAAA | C |
| ATOM | 543 | CD1 | TYR | A | 62 | 99.370 | 215.824 | −34.693 | 1.00 | 50.00 | AAAA | C |
| ATOM | 544 | CD2 | TYR | A | 62 | 98.800 | 213.460 | −34.437 | 1.00 | 50.00 | AAAA | C |
| ATOM | 545 | CE1 | TYR | A | 62 | 100.710 | 215.461 | −34.954 | 1.00 | 50.00 | AAAA | C |
| ATOM | 546 | CE2 | TYR | A | 62 | 100.124 | 213.089 | −34.689 | 1.00 | 50.00 | AAAA | C |
| ATOM | 547 | CZ | TYR | A | 62 | 101.066 | 214.093 | −34.957 | 1.00 | 50.00 | AAAA | C |
| ATOM | 548 | OH | TYR | A | 62 | 102.363 | 213.709 | −35.234 | 1.00 | 50.00 | AAAA | O |
| ATOM | 549 | H | TYR | A | 62 | 94.473 | 215.548 | −33.974 | 0.00 | 0.00 | AAAA | H |
| ATOM | 550 | HH | TYR | A | 62 | 102.400 | 212.766 | −35.355 | 0.00 | 0.00 | AAAA | H |
| ATOM | 551 | N | ILE | A | 63 | 95.179 | 213.123 | −36.075 | 1.00 | 50.00 | AAAA | N |
| ATOM | 552 | CA | ILE | A | 63 | 94.891 | 212.050 | −37.045 | 1.00 | 50.00 | AAAA | C |
| ATOM | 553 | C | ILE | A | 63 | 94.533 | 212.571 | −38.412 | 1.00 | 50.00 | AAAA | C |
| ATOM | 554 | O | ILE | A | 63 | 95.143 | 212.217 | −39.410 | 1.00 | 50.00 | AAAA | O |
| ATOM | 555 | CB | ILE | A | 63 | 93.806 | 211.049 | −36.551 | 1.00 | 50.00 | AAAA | C |
| ATOM | 556 | CG1 | ILE | A | 63 | 94.474 | 209.926 | −35.774 | 1.00 | 50.00 | AAAA | C |
| ATOM | 557 | CG2 | ILE | A | 63 | 92.994 | 210.343 | −37.658 | 1.00 | 50.00 | AAAA | C |
| ATOM | 558 | CD1 | ILE | A | 63 | 95.385 | 209.089 | −36.683 | 1.00 | 50.00 | AAAA | C |
| ATOM | 559 | H | ILE | A | 63 | 94.759 | 213.101 | −35.174 | 0.00 | 0.00 | AAAA | H |
| ATOM | 560 | N | ASP | A | 64 | 93.522 | 213.450 | −38.395 | 1.00 | 50.00 | AAAA | N |
| ATOM | 561 | CA | ASP | A | 64 | 92.998 | 214.021 | −39.631 | 1.00 | 50.00 | AAAA | C |
| ATOM | 562 | C | ASP | A | 64 | 94.076 | 214.713 | −40.444 | 1.00 | 50.00 | AAAA | C |
| ATOM | 563 | O | ASP | A | 64 | 94.145 | 214.600 | −41.659 | 1.00 | 50.00 | AAAA | O |
| ATOM | 564 | CB | ASP | A | 64 | 91.860 | 214.986 | −39.280 | 1.00 | 50.00 | AAAA | C |
| ATOM | 565 | CG | ASP | A | 64 | 90.977 | 215.322 | −40.472 | 1.00 | 50.00 | AAAA | C |
| ATOM | 566 | OD1 | ASP | A | 64 | 91.480 | 215.516 | −41.578 | 1.00 | 50.00 | AAAA | O |
| ATOM | 567 | OD2 | ASP | A | 64 | 89.766 | 215.389 | −40.281 | 1.00 | 50.00 | AAAA | O |
| ATOM | 568 | H | ASP | A | 64 | 93.111 | 213.661 | −37.511 | 0.00 | 0.00 | AAAA | H |
| ATOM | 569 | N | LYS | A | 65 | 94.935 | 215.413 | −39.684 | 1.00 | 50.00 | AAAA | N |
| ATOM | 570 | CA | LYS | A | 65 | 96.049 | 216.124 | −40.303 | 1.00 | 50.00 | AAAA | C |
| ATOM | 571 | C | LYS | A | 65 | 97.026 | 215.237 | −41.066 | 1.00 | 50.00 | AAAA | C |
| ATOM | 572 | O | LYS | A | 65 | 97.480 | 215.563 | −42.155 | 1.00 | 50.00 | AAAA | O |
| ATOM | 573 | CB | LYS | A | 65 | 96.761 | 216.951 | −39.227 | 1.00 | 50.00 | AAAA | C |
| ATOM | 574 | CG | LYS | A | 65 | 97.831 | 217.904 | −39.764 | 1.00 | 50.00 | AAAA | C |
| ATOM | 575 | CD | LYS | A | 65 | 97.255 | 218.968 | −40.701 | 1.00 | 50.00 | AAAA | C |
| ATOM | 576 | CE | LYS | A | 65 | 98.332 | 219.866 | −41.315 | 1.00 | 50.00 | AAAA | C |
| ATOM | 577 | NZ | LYS | A | 65 | 99.199 | 219.079 | −42.207 | 1.00 | 50.00 | AAAA | N |
| ATOM | 578 | H | LYS | A | 65 | 94.819 | 215.413 | −38.690 | 0.00 | 0.00 | AAAA | H |
| ATOM | 579 | 1HZ | LYS | A | 65 | 99.939 | 219.699 | −42.594 | 0.00 | 0.00 | AAAA | H |
| ATOM | 580 | 2HZ | LYS | A | 65 | 98.625 | 218.696 | −42.986 | 0.00 | 0.00 | AAAA | H |
| ATOM | 581 | 3HZ | LYS | A | 65 | 99.634 | 218.301 | −41.673 | 0.00 | 0.00 | AAAA | H |
| ATOM | 582 | N | GLN | A | 66 | 97.333 | 214.093 | −40.435 | 1.00 | 50.00 | AAAA | N |
| ATOM | 583 | CA | GLN | A | 66 | 98.384 | 213.268 | −41.023 | 1.00 | 50.00 | AAAA | C |
| ATOM | 584 | C | GLN | A | 66 | 97.932 | 212.234 | −42.050 | 1.00 | 50.00 | AAAA | C |
| ATOM | 585 | O | GLN | A | 66 | 98.580 | 212.020 | −43.067 | 1.00 | 50.00 | AAAA | O |
| ATOM | 586 | CB | GLN | A | 66 | 99.224 | 212.643 | −39.910 | 1.00 | 50.00 | AAAA | C |
| ATOM | 587 | CG | GLN | A | 66 | 98.374 | 211.841 | −38.929 | 1.00 | 50.00 | AAAA | C |
| ATOM | 588 | CD | GLN | A | 66 | 99.255 | 211.169 | −37.924 | 1.00 | 50.00 | AAAA | C |
| ATOM | 589 | NE2 | GLN | A | 66 | 98.991 | 209.868 | −37.816 | 1.00 | 50.00 | AAAA | N |
| ATOM | 590 | OE1 | GLN | A | 66 | 100.107 | 211.763 | −37.281 | 1.00 | 50.00 | AAAA | O |
| ATOM | 591 | H | GLN | A | 66 | 96.860 | 213.846 | −39.589 | 0.00 | 0.00 | AAAA | H |
| ATOM | 592 | 1HE2 | GLN | A | 66 | 99.430 | 209.386 | −37.059 | 0.00 | 0.00 | AAAA | H |
| ATOM | 593 | 2HE2 | GLN | A | 66 | 98.399 | 209.370 | −38.450 | 0.00 | 0.00 | AAAA | H |
| ATOM | 594 | N | LEU | A | 67 | 96.789 | 211.597 | −41.730 | 1.00 | 50.00 | AAAA | N |
| ATOM | 595 | CA | LEU | A | 67 | 96.267 | 210.500 | −42.535 | 1.00 | 50.00 | AAAA | C |
| ATOM | 596 | C | LEU | A | 67 | 95.920 | 210.884 | −43.965 | 1.00 | 50.00 | AAAA | C |
| ATOM | 597 | O | LEU | A | 67 | 95.216 | 211.848 | −44.234 | 1.00 | 50.00 | AAAA | O |
| ATOM | 598 | CB | LEU | A | 67 | 95.097 | 209.864 | −41.764 | 1.00 | 50.00 | AAAA | C |
| ATOM | 599 | CG | LEU | A | 67 | 94.393 | 208.644 | −42.368 | 1.00 | 50.00 | AAAA | C |
| ATOM | 600 | CD1 | LEU | A | 67 | 93.944 | 207.687 | −41.269 | 1.00 | 50.00 | AAAA | C |
| ATOM | 601 | CD2 | LEU | A | 67 | 93.207 | 209.014 | −43.257 | 1.00 | 50.00 | AAAA | C |
| ATOM | 602 | H | LEU | A | 67 | 96.307 | 211.876 | −40.903 | 0.00 | 0.00 | AAAA | H |
| ATOM | 603 | N | LEU | A | 68 | 96.464 | 210.047 | −44.870 | 1.00 | 50.00 | AAAA | N |
| ATOM | 604 | CA | LEU | A | 68 | 96.281 | 210.283 | −46.302 | 1.00 | 50.00 | AAAA | C |
| ATOM | 605 | C | LEU | A | 68 | 94.837 | 210.035 | −46.720 | 1.00 | 50.00 | AAAA | C |
| ATOM | 606 | O | LEU | A | 68 | 94.229 | 209.086 | −46.244 | 1.00 | 50.00 | AAAA | O |
| ATOM | 607 | CB | LEU | A | 68 | 97.222 | 209.337 | −47.068 | 1.00 | 50.00 | AAAA | C |
| ATOM | 608 | CG | LEU | A | 68 | 97.435 | 209.680 | −48.548 | 1.00 | 50.00 | AAAA | C |
| ATOM | 609 | CD1 | LEU | A | 68 | 98.291 | 210.930 | −48.736 | 1.00 | 50.00 | AAAA | C |
| ATOM | 610 | CD2 | LEU | A | 68 | 98.021 | 208.514 | −49.334 | 1.00 | 50.00 | AAAA | C |
| ATOM | 611 | H | LEU | A | 68 | 96.985 | 209.254 | −44.555 | 0.00 | 0.00 | AAAA | H |
| ATOM | 612 | N | PRO | A | 69 | 94.310 | 210.895 | −47.637 | 1.00 | 50.00 | AAAA | N |
| ATOM | 613 | CA | PRO | A | 69 | 92.988 | 210.675 | −48.246 | 1.00 | 50.00 | AAAA | C |
| ATOM | 614 | C | PRO | A | 69 | 92.567 | 209.231 | −48.516 | 1.00 | 50.00 | AAAA | C |
| ATOM | 615 | O | PRO | A | 69 | 91.412 | 208.866 | −48.341 | 1.00 | 50.00 | AAAA | O |
| ATOM | 616 | CB | PRO | A | 69 | 93.079 | 211.512 | −49.524 | 1.00 | 50.00 | AAAA | C |
| ATOM | 617 | CG | PRO | A | 69 | 93.929 | 212.713 | −49.123 | 1.00 | 50.00 | AAAA | C |
| ATOM | 618 | CD | PRO | A | 69 | 94.931 | 212.123 | −48.137 | 1.00 | 50.00 | AAAA | C |
| ATOM | 619 | N | ILE | A | 70 | 93.568 | 208.433 | −48.951 | 1.00 | 50.00 | AAAA | N |
| ATOM | 620 | CA | ILE | A | 70 | 93.345 | 207.002 | −49.172 | 1.00 | 50.00 | AAAA | C |
| ATOM | 621 | C | ILE | A | 70 | 94.526 | 206.188 | −48.648 | 1.00 | 50.00 | AAAA | C |
| ATOM | 622 | O | ILE | A | 70 | 95.682 | 206.483 | −48.923 | 1.00 | 50.00 | AAAA | O |
| ATOM | 623 | CB | ILE | A | 70 | 93.089 | 206.687 | −50.662 | 1.00 | 50.00 | AAAA | C |
| ATOM | 624 | CG1 | ILE | A | 70 | 94.198 | 207.217 | −51.582 | 1.00 | 50.00 | AAAA | C |
| ATOM | 625 | CG2 | ILE | A | 70 | 91.725 | 207.225 | −51.113 | 1.00 | 50.00 | AAAA | C |
| ATOM | 626 | CD1 | ILE | A | 70 | 94.045 | 206.756 | −53.032 | 1.00 | 50.00 | AAAA | C |
| ATOM | 627 | H | ILE | A | 70 | 94.479 | 208.815 | −49.093 | 0.00 | 0.00 | AAAA | H |
| ATOM | 628 | N | VAL | A | 71 | 94.204 | 205.146 | −47.862 | 1.00 | 50.00 | AAAA | N |
| ATOM | 629 | CA | VAL | A | 71 | 95.300 | 204.245 | −47.501 | 1.00 | 50.00 | AAAA | C |
| ATOM | 630 | C | VAL | A | 71 | 95.405 | 203.053 | −48.450 | 1.00 | 50.00 | AAAA | C |
| ATOM | 631 | O | VAL | A | 71 | 94.436 | 202.356 | −48.729 | 1.00 | 50.00 | AAAA | O |
| ATOM | 632 | CB | VAL | A | 71 | 95.216 | 203.849 | −46.009 | 1.00 | 50.00 | AAAA | C |
| ATOM | 633 | CG1 | VAL | A | 71 | 93.902 | 203.149 | −45.640 | 1.00 | 50.00 | AAAA | C |
| ATOM | 634 | CG2 | VAL | A | 71 | 96.452 | 203.067 | −45.549 | 1.00 | 50.00 | AAAA | C |
| ATOM | 635 | H | VAL | A | 71 | 93.257 | 204.942 | −47.613 | 0.00 | 0.00 | AAAA | H |
| ATOM | 636 | N | ASN | A | 72 | 96.634 | 202.884 | −48.977 | 1.00 | 50.00 | AAAA | N |
| ATOM | 637 | CA | ASN | A | 72 | 96.862 | 201.786 | −49.918 | 1.00 | 50.00 | AAAA | C |
| ATOM | 638 | C | ASN | A | 72 | 98.068 | 200.969 | −49.487 | 1.00 | 50.00 | AAAA | C |
| ATOM | 639 | O | ASN | A | 72 | 98.809 | 201.390 | −48.618 | 1.00 | 50.00 | AAAA | O |
| ATOM | 640 | CB | ASN | A | 72 | 96.973 | 202.344 | −51.366 | 1.00 | 50.00 | AAAA | C |
| ATOM | 641 | CG | ASN | A | 72 | 96.921 | 201.273 | −52.465 | 1.00 | 50.00 | AAAA | C |
| ATOM | 642 | ND2 | ASN | A | 72 | 95.753 | 201.183 | −53.124 | 1.00 | 50.00 | AAAA | N |
| ATOM | 643 | OD1 | ASN | A | 72 | 97.878 | 200.554 | −52.708 | 1.00 | 50.00 | AAAA | O |
| ATOM | 644 | H | ASN | A | 72 | 97.391 | 203.471 | −48.692 | 0.00 | 0.00 | AAAA | H |
| ATOM | 645 | 1HD2 | ASN | A | 72 | 95.620 | 200.433 | −53.775 | 0.00 | 0.00 | AAAA | H |
| ATOM | 646 | 2HD2 | ASN | A | 72 | 94.981 | 201.805 | −52.996 | 0.00 | 0.00 | AAAA | H |
| ATOM | 647 | N | LYS | A | 73 | 98.272 | 199.815 | −50.155 | 1.00 | 50.00 | AAAA | N |
| ATOM | 648 | CA | LYS | A | 73 | 99.563 | 199.108 | −50.126 | 1.00 | 50.00 | AAAA | C |
| ATOM | 649 | C | LYS | A | 73 | 100.811 | 199.991 | −50.269 | 1.00 | 50.00 | AAAA | C |
| ATOM | 650 | O | LYS | A | 73 | 101.862 | 199.729 | −49.695 | 1.00 | 50.00 | AAAA | O |
| ATOM | 651 | CB | LYS | A | 73 | 99.532 | 198.022 | −51.211 | 1.00 | 50.00 | AAAA | C |
| ATOM | 652 | CG | LYS | A | 73 | 100.715 | 197.050 | −51.206 | 1.00 | 50.00 | AAAA | C |
| ATOM | 653 | CD | LYS | A | 73 | 100.784 | 196.224 | −49.922 | 1.00 | 50.00 | AAAA | C |
| ATOM | 654 | CE | LYS | A | 73 | 102.020 | 195.325 | −49.857 | 1.00 | 50.00 | AAAA | C |
| ATOM | 655 | NZ | LYS | A | 73 | 103.239 | 196.147 | −49.803 | 1.00 | 50.00 | AAAA | N |
| ATOM | 656 | H | LYS | A | 73 | 97.533 | 199.432 | −50.708 | 0.00 | 0.00 | AAAA | H |
| ATOM | 657 | 1HZ | LYS | A | 73 | 104.072 | 195.527 | −49.753 | 0.00 | 0.00 | AAAA | H |
| ATOM | 658 | 2HZ | LYS | A | 73 | 103.206 | 196.747 | −48.954 | 0.00 | 0.00 | AAAA | H |
| ATOM | 659 | 3HZ | LYS | A | 73 | 103.301 | 196.745 | −50.650 | 0.00 | 0.00 | AAAA | H |
| ATOM | 660 | N | GLN | A | 74 | 100.631 | 201.074 | −51.057 | 1.00 | 50.00 | AAAA | N |
| ATOM | 661 | CA | GLN | A | 74 | 101.703 | 202.063 | −51.194 | 1.00 | 50.00 | AAAA | C |
| ATOM | 662 | C | GLN | A | 74 | 102.003 | 202.857 | −49.926 | 1.00 | 50.00 | AAAA | C |
| ATOM | 663 | O | GLN | A | 74 | 103.149 | 203.090 | −49.565 | 1.00 | 50.00 | AAAA | O |
| ATOM | 664 | CB | GLN | A | 74 | 101.414 | 202.999 | −52.379 | 1.00 | 50.00 | AAAA | C |
| ATOM | 665 | CG | GLN | A | 74 | 100.156 | 203.863 | −52.219 | 1.00 | 50.00 | AAAA | C |
| ATOM | 666 | CD | GLN | A | 74 | 99.943 | 204.741 | −53.424 | 1.00 | 50.00 | AAAA | C |
| ATOM | 667 | NE2 | GLN | A | 74 | 99.964 | 206.054 | −53.141 | 1.00 | 50.00 | AAAA | N |
| ATOM | 668 | OE1 | GLN | A | 74 | 99.751 | 204.270 | −54.535 | 1.00 | 50.00 | AAAA | O |
| ATOM | 669 | H | GLN | A | 74 | 99.750 | 201.197 | −51.514 | 0.00 | 0.00 | AAAA | H |
| ATOM | 670 | 1HE2 | GLN | A | 74 | 99.816 | 206.730 | −53.863 | 0.00 | 0.00 | AAAA | H |
| ATOM | 671 | 2HE2 | GLN | A | 74 | 100.127 | 206.371 | −52.206 | 0.00 | 0.00 | AAAA | H |
| ATOM | 672 | N | SER | A | 75 | 100.905 | 203.261 | −49.262 | 1.00 | 50.00 | AAAA | N |
| ATOM | 673 | CA | SER | A | 75 | 101.050 | 204.090 | −48.077 | 1.00 | 50.00 | AAAA | C |
| ATOM | 674 | C | SER | A | 75 | 101.400 | 203.292 | −46.850 | 1.00 | 50.00 | AAAA | C |
| ATOM | 675 | O | SER | A | 75 | 102.327 | 203.624 | −46.136 | 1.00 | 50.00 | AAAA | O |
| ATOM | 676 | CB | SER | A | 75 | 99.780 | 204.905 | −47.851 | 1.00 | 50.00 | AAAA | C |
| ATOM | 677 | OG | SER | A | 75 | 99.435 | 205.588 | −49.055 | 1.00 | 50.00 | AAAA | O |
| ATOM | 678 | H | SER | A | 75 | 100.001 | 202.964 | −49.565 | 0.00 | 0.00 | AAAA | H |
| ATOM | 679 | HG | SER | A | 75 | 98.789 | 206.236 | −48.809 | 0.00 | 0.00 | AAAA | H |
| ATOM | 680 | N | CYS | A | 76 | 100.630 | 202.205 | −46.666 | 1.00 | 50.00 | AAAA | N |
| ATOM | 681 | CA | CYS | A | 76 | 100.748 | 201.244 | −45.571 | 1.00 | 50.00 | AAAA | C |
| ATOM | 682 | C | CYS | A | 76 | 101.814 | 201.447 | −44.523 | 1.00 | 50.00 | AAAA | C |
| ATOM | 683 | O | CYS | A | 76 | 101.517 | 201.812 | −43.400 | 1.00 | 50.00 | AAAA | O |
| ATOM | 684 | CB | CYS | A | 76 | 100.853 | 199.812 | −46.077 | 1.00 | 50.00 | AAAA | C |
| ATOM | 685 | SG | CYS | A | 76 | 100.243 | 198.773 | −44.741 | 1.00 | 50.00 | AAAA | S |
| ATOM | 686 | H | CYS | A | 76 | 99.904 | 202.087 | −47.334 | 0.00 | 0.00 | AAAA | H |
| ATOM | 687 | N | SER | A | 77 | 103.064 | 201.189 | −44.955 | 1.00 | 50.00 | AAAA | N |
| ATOM | 688 | CA | SER | A | 77 | 104.217 | 201.370 | −44.069 | 1.00 | 50.00 | AAAA | C |
| ATOM | 689 | C | SER | A | 77 | 104.241 | 202.732 | −43.370 | 1.00 | 50.00 | AAAA | C |
| ATOM | 690 | O | SER | A | 77 | 104.274 | 202.824 | −42.153 | 1.00 | 50.00 | AAAA | O |
| ATOM | 691 | CB | SER | A | 77 | 105.491 | 201.093 | −44.879 | 1.00 | 50.00 | AAAA | C |
| ATOM | 692 | OG | SER | A | 77 | 106.632 | 200.953 | −44.027 | 1.00 | 50.00 | AAAA | O |
| ATOM | 693 | H | SER | A | 77 | 103.182 | 200.890 | −45.902 | 0.00 | 0.00 | AAAA | H |
| ATOM | 694 | HG | SER | A | 77 | 107.388 | 200.876 | −44.598 | 0.00 | 0.00 | AAAA | H |
| ATOM | 695 | N | ILE | A | 78 | 104.157 | 203.780 | −44.212 | 1.00 | 50.00 | AAAA | N |
| ATOM | 696 | CA | ILE | A | 78 | 104.147 | 205.158 | −43.706 | 1.00 | 50.00 | AAAA | C |
| ATOM | 697 | C | ILE | A | 78 | 102.884 | 205.643 | −42.989 | 1.00 | 50.00 | AAAA | C |
| ATOM | 698 | O | ILE | A | 78 | 102.943 | 206.515 | −42.134 | 1.00 | 50.00 | AAAA | O |
| ATOM | 699 | CB | ILE | A | 78 | 104.566 | 206.170 | −44.792 | 1.00 | 50.00 | AAAA | C |
| ATOM | 700 | CG1 | ILE | A | 78 | 103.468 | 206.391 | −45.844 | 1.00 | 50.00 | AAAA | C |
| ATOM | 701 | CG2 | ILE | A | 78 | 105.879 | 205.704 | −45.435 | 1.00 | 50.00 | AAAA | C |
| ATOM | 702 | CD1 | ILE | A | 78 | 103.768 | 207.454 | −46.898 | 1.00 | 50.00 | AAAA | C |
| ATOM | 703 | H | ILE | A | 78 | 104.070 | 203.609 | −45.192 | 0.00 | 0.00 | AAAA | H |
| ATOM | 704 | N | SER | A | 79 | 101.731 | 205.051 | −43.359 | 1.00 | 50.00 | AAAA | N |
| ATOM | 705 | CA | SER | A | 79 | 100.501 | 205.492 | −42.693 | 1.00 | 50.00 | AAAA | C |
| ATOM | 706 | C | SER | A | 79 | 100.310 | 204.898 | −41.313 | 1.00 | 50.00 | AAAA | C |
| ATOM | 707 | O | SER | A | 79 | 99.864 | 205.530 | −40.367 | 1.00 | 50.00 | AAAA | O |
| ATOM | 708 | CB | SER | A | 79 | 99.282 | 205.203 | −43.565 | 1.00 | 50.00 | AAAA | C |
| ATOM | 709 | OG | SER | A | 79 | 99.352 | 205.995 | −44.752 | 1.00 | 50.00 | AAAA | O |
| ATOM | 710 | H | SER | A | 79 | 101.722 | 204.331 | −44.052 | 0.00 | 0.00 | AAAA | H |
| ATOM | 711 | HG | SER | A | 79 | 98.712 | 205.643 | −45.356 | 0.00 | 0.00 | AAAA | H |
| ATOM | 712 | N | ASN | A | 80 | 100.722 | 203.627 | −41.259 | 1.00 | 50.00 | AAAA | N |
| ATOM | 713 | CA | ASN | A | 80 | 100.797 | 202.879 | −40.010 | 1.00 | 50.00 | AAAA | C |
| ATOM | 714 | C | ASN | A | 80 | 101.758 | 203.516 | −39.006 | 1.00 | 50.00 | AAAA | C |
| ATOM | 715 | O | ASN | A | 80 | 101.459 | 203.624 | −37.826 | 1.00 | 50.00 | AAAA | O |
| ATOM | 716 | CB | ASN | A | 80 | 101.151 | 201.454 | −40.458 | 1.00 | 50.00 | AAAA | C |
| ATOM | 717 | CG | ASN | A | 80 | 101.159 | 200.401 | −39.386 | 1.00 | 50.00 | AAAA | C |
| ATOM | 718 | ND2 | ASN | A | 80 | 99.991 | 199.769 | −39.233 | 1.00 | 50.00 | AAAA | N |
| ATOM | 719 | OD1 | ASN | A | 80 | 102.174 | 200.134 | −38.766 | 1.00 | 50.00 | AAAA | O |
| ATOM | 720 | H | ASN | A | 80 | 101.046 | 203.208 | −42.105 | 0.00 | 0.00 | AAAA | H |
| ATOM | 721 | 1HD2 | ASN | A | 80 | 99.869 | 199.160 | −38.451 | 0.00 | 0.00 | AAAA | H |
| ATOM | 722 | 2HD2 | ASN | A | 80 | 99.257 | 199.862 | −39.904 | 0.00 | 0.00 | AAAA | H |
| ATOM | 723 | N | ILE | A | 81 | 102.912 | 203.981 | −39.541 | 1.00 | 50.00 | AAAA | N |
| ATOM | 724 | CA | ILE | A | 81 | 103.880 | 204.617 | −38.635 | 1.00 | 50.00 | AAAA | C |
| ATOM | 725 | C | ILE | A | 81 | 103.499 | 205.977 | −38.065 | 1.00 | 50.00 | AAAA | C |
| ATOM | 726 | O | ILE | A | 81 | 103.881 | 206.338 | −36.961 | 1.00 | 50.00 | AAAA | O |
| ATOM | 727 | CB | ILE | A | 81 | 105.306 | 204.693 | −39.205 | 1.00 | 50.00 | AAAA | C |
| ATOM | 728 | CG1 | ILE | A | 81 | 105.384 | 205.627 | −40.415 | 1.00 | 50.00 | AAAA | C |
| ATOM | 729 | CG2 | ILE | A | 81 | 105.805 | 203.283 | −39.524 | 1.00 | 50.00 | AAAA | C |
| ATOM | 730 | CD1 | ILE | A | 81 | 106.784 | 205.893 | −40.964 | 1.00 | 50.00 | AAAA | C |
| ATOM | 731 | H | ILE | A | 81 | 103.074 | 203.904 | −40.524 | 0.00 | 0.00 | AAAA | H |
| ATOM | 732 | N | GLU | A | 82 | 102.726 | 206.734 | −38.852 | 1.00 | 50.00 | AAAA | N |
| ATOM | 733 | CA | GLU | A | 82 | 102.333 | 208.025 | −38.292 | 1.00 | 50.00 | AAAA | C |
| ATOM | 734 | C | GLU | A | 82 | 101.176 | 207.896 | −37.314 | 1.00 | 50.00 | AAAA | C |
| ATOM | 735 | O | GLU | A | 82 | 101.123 | 208.525 | −36.265 | 1.00 | 50.00 | AAAA | O |
| ATOM | 736 | CB | GLU | A | 82 | 102.057 | 209.031 | −39.411 | 1.00 | 50.00 | AAAA | C |
| ATOM | 737 | CG | GLU | A | 82 | 101.089 | 208.463 | −40.447 | 1.00 | 50.00 | AAAA | C |
| ATOM | 738 | CD | GLU | A | 82 | 100.778 | 209.443 | −41.541 | 1.00 | 50.00 | AAAA | C |
| ATOM | 739 | OE1 | GLU | A | 82 | 101.688 | 209.853 | −42.260 | 1.00 | 50.00 | AAAA | O |
| ATOM | 740 | OE2 | GLU | A | 82 | 99.609 | 209.788 | −41.671 | 1.00 | 50.00 | AAAA | O |
| ATOM | 741 | H | GLU | A | 82 | 102.379 | 206.375 | −39.718 | 0.00 | 0.00 | AAAA | H |
| ATOM | 742 | N | THR | A | 83 | 100.253 | 206.998 | −37.702 | 1.00 | 50.00 | AAAA | N |
| ATOM | 743 | CA | THR | A | 83 | 99.125 | 206.663 | −36.842 | 1.00 | 50.00 | AAAA | C |
| ATOM | 744 | C | THR | A | 83 | 99.561 | 205.925 | −35.582 | 1.00 | 50.00 | AAAA | C |
| ATOM | 745 | O | THR | A | 83 | 98.915 | 205.976 | −34.543 | 1.00 | 50.00 | AAAA | O |
| ATOM | 746 | CB | THR | A | 83 | 98.094 | 205.893 | −37.684 | 1.00 | 50.00 | AAAA | C |
| ATOM | 747 | CG2 | THR | A | 83 | 96.816 | 205.517 | −36.932 | 1.00 | 50.00 | AAAA | C |
| ATOM | 748 | OG1 | THR | A | 83 | 97.769 | 206.667 | −38.844 | 1.00 | 50.00 | AAAA | O |
| ATOM | 749 | H | THR | A | 83 | 100.360 | 206.517 | −38.572 | 0.00 | 0.00 | AAAA | H |
| ATOM | 750 | HG1 | THR | A | 83 | 96.997 | 206.268 | −39.223 | 0.00 | 0.00 | AAAA | H |
| ATOM | 751 | N | VAL | A | 84 | 100.738 | 205.277 | −35.711 | 1.00 | 50.00 | AAAA | N |
| ATOM | 752 | CA | VAL | A | 84 | 101.275 | 204.670 | −34.500 | 1.00 | 50.00 | AAAA | C |
| ATOM | 753 | C | VAL | A | 84 | 101.802 | 205.642 | −33.491 | 1.00 | 50.00 | AAAA | C |
| ATOM | 754 | O | VAL | A | 84 | 101.415 | 205.594 | −32.336 | 1.00 | 50.00 | AAAA | O |
| ATOM | 755 | CB | VAL | A | 84 | 102.297 | 203.545 | −34.734 | 1.00 | 50.00 | AAAA | C |
| ATOM | 756 | CG1 | VAL | A | 84 | 103.692 | 203.888 | −35.242 | 1.00 | 50.00 | AAAA | C |
| ATOM | 757 | CG2 | VAL | A | 84 | 102.428 | 202.734 | −33.460 | 1.00 | 50.00 | AAAA | C |
| ATOM | 758 | H | VAL | A | 84 | 101.220 | 205.268 | −36.587 | 0.00 | 0.00 | AAAA | H |
| ATOM | 759 | N | ILE | A | 85 | 102.686 | 206.535 | −33.975 | 1.00 | 50.00 | AAAA | N |
| ATOM | 760 | CA | ILE | A | 85 | 103.374 | 207.386 | −33.007 | 1.00 | 50.00 | AAAA | C |
| ATOM | 761 | C | ILE | A | 85 | 102.399 | 208.219 | −32.215 | 1.00 | 50.00 | AAAA | C |
| ATOM | 762 | O | ILE | A | 85 | 102.533 | 208.440 | −31.025 | 1.00 | 50.00 | AAAA | O |
| ATOM | 763 | CB | ILE | A | 85 | 104.454 | 208.248 | −33.674 | 1.00 | 50.00 | AAAA | C |
| ATOM | 764 | CG1 | ILE | A | 85 | 103.901 | 209.168 | −34.770 | 1.00 | 50.00 | AAAA | C |
| ATOM | 765 | CG2 | ILE | A | 85 | 105.538 | 207.318 | −34.225 | 1.00 | 50.00 | AAAA | C |
| ATOM | 766 | CD1 | ILE | A | 85 | 104.917 | 210.167 | −35.319 | 1.00 | 50.00 | AAAA | C |
| ATOM | 767 | H | ILE | A | 85 | 102.872 | 206.591 | −34.954 | 0.00 | 0.00 | AAAA | H |
| ATOM | 768 | N | GLU | A | 86 | 101.339 | 208.581 | −32.949 | 1.00 | 50.00 | AAAA | N |
| ATOM | 769 | CA | GLU | A | 86 | 100.252 | 209.314 | −32.344 | 1.00 | 50.00 | AAAA | C |
| ATOM | 770 | C | GLU | A | 86 | 99.518 | 208.558 | −31.246 | 1.00 | 50.00 | AAAA | C |
| ATOM | 771 | O | GLU | A | 86 | 99.372 | 209.028 | −30.124 | 1.00 | 50.00 | AAAA | O |
| ATOM | 772 | CB | GLU | A | 86 | 99.337 | 209.702 | −33.483 | 1.00 | 50.00 | AAAA | C |
| ATOM | 773 | CG | GLU | A | 86 | 98.116 | 210.426 | −32.958 | 1.00 | 50.00 | AAAA | C |
| ATOM | 774 | CD | GLU | A | 86 | 97.272 | 210.967 | −34.073 | 1.00 | 50.00 | AAAA | C |
| ATOM | 775 | OE1 | GLU | A | 86 | 97.596 | 210.784 | −35.244 | 1.00 | 50.00 | AAAA | O |
| ATOM | 776 | OE2 | GLU | A | 86 | 96.291 | 211.631 | −33.756 | 1.00 | 50.00 | AAAA | O |
| ATOM | 777 | H | GLU | A | 86 | 101.290 | 208.327 | −33.917 | 0.00 | 0.00 | AAAA | H |
| ATOM | 778 | N | PHE | A | 87 | 99.051 | 207.356 | −31.637 | 1.00 | 50.00 | AAAA | N |
| ATOM | 779 | CA | PHE | A | 87 | 98.249 | 206.562 | −30.704 | 1.00 | 50.00 | AAAA | C |
| ATOM | 780 | C | PHE | A | 87 | 99.021 | 206.107 | −29.477 | 1.00 | 50.00 | AAAA | C |
| ATOM | 781 | O | PHE | A | 87 | 98.509 | 206.076 | −28.370 | 1.00 | 50.00 | AAAA | O |
| ATOM | 782 | CB | PHE | A | 87 | 97.636 | 205.343 | −31.410 | 1.00 | 50.00 | AAAA | C |
| ATOM | 783 | CG | PHE | A | 87 | 96.509 | 205.680 | −32.375 | 1.00 | 50.00 | AAAA | C |
| ATOM | 784 | CD1 | PHE | A | 87 | 96.437 | 206.934 | −33.021 | 1.00 | 50.00 | AAAA | C |
| ATOM | 785 | CD2 | PHE | A | 87 | 95.533 | 204.689 | −32.624 | 1.00 | 50.00 | AAAA | C |
| ATOM | 786 | CE1 | PHE | A | 87 | 95.390 | 207.196 | −33.917 | 1.00 | 50.00 | AAAA | C |
| ATOM | 787 | CE2 | PHE | A | 87 | 94.484 | 204.949 | −33.526 | 1.00 | 50.00 | AAAA | C |
| ATOM | 788 | CZ | PHE | A | 87 | 94.424 | 206.202 | −34.169 | 1.00 | 50.00 | AAAA | C |
| ATOM | 789 | H | PHE | A | 87 | 99.237 | 207.023 | −32.562 | 0.00 | 0.00 | AAAA | H |
| ATOM | 790 | N | GLN | A | 88 | 100.294 | 205.776 | −29.752 | 1.00 | 50.00 | AAAA | N |
| ATOM | 791 | CA | GLN | A | 88 | 101.263 | 205.374 | −28.732 | 1.00 | 50.00 | AAAA | C |
| ATOM | 792 | C | GLN | A | 88 | 101.673 | 206.471 | −27.778 | 1.00 | 50.00 | AAAA | C |
| ATOM | 793 | O | GLN | A | 88 | 101.895 | 206.233 | −26.597 | 1.00 | 50.00 | AAAA | O |
| ATOM | 794 | CB | GLN | A | 88 | 102.542 | 204.845 | −29.363 | 1.00 | 50.00 | AAAA | C |
| ATOM | 795 | CG | GLN | A | 88 | 102.416 | 203.456 | −29.964 | 1.00 | 50.00 | AAAA | C |
| ATOM | 796 | CD | GLN | A | 88 | 103.741 | 203.096 | −30.604 | 1.00 | 50.00 | AAAA | C |
| ATOM | 797 | NE2 | GLN | A | 88 | 104.046 | 201.797 | −30.485 | 1.00 | 50.00 | AAAA | N |
| ATOM | 798 | OE1 | GLN | A | 88 | 104.432 | 203.919 | −31.193 | 1.00 | 50.00 | AAAA | O |
| ATOM | 799 | H | GLN | A | 88 | 100.567 | 205.874 | −30.703 | 0.00 | 0.00 | AAAA | H |
| ATOM | 800 | 1HE2 | GLN | A | 88 | 104.881 | 201.446 | −30.905 | 0.00 | 0.00 | AAAA | H |
| ATOM | 801 | 2HE2 | GLN | A | 88 | 103.433 | 201.166 | −30.012 | 0.00 | 0.00 | AAAA | H |
| ATOM | 802 | N | GLN | A | 89 | 101.765 | 207.692 | −28.342 | 1.00 | 50.00 | AAAA | N |
| ATOM | 803 | CA | GLN | A | 89 | 102.080 | 208.829 | −27.474 | 1.00 | 50.00 | AAAA | C |
| ATOM | 804 | C | GLN | A | 89 | 100.952 | 209.127 | −26.512 | 1.00 | 50.00 | AAAA | C |
| ATOM | 805 | O | GLN | A | 89 | 101.154 | 209.415 | −25.341 | 1.00 | 50.00 | AAAA | O |
| ATOM | 806 | CB | GLN | A | 89 | 102.422 | 210.076 | −28.284 | 1.00 | 50.00 | AAAA | C |
| ATOM | 807 | CG | GLN | A | 89 | 103.838 | 210.014 | −28.859 | 1.00 | 50.00 | AAAA | C |
| ATOM | 808 | CD | GLN | A | 89 | 104.047 | 211.159 | −29.828 | 1.00 | 50.00 | AAAA | C |
| ATOM | 809 | NE2 | GLN | A | 89 | 105.200 | 211.807 | −29.621 | 1.00 | 50.00 | AAAA | N |
| ATOM | 810 | OE1 | GLN | A | 89 | 103.234 | 211.456 | −30.693 | 1.00 | 50.00 | AAAA | O |
| ATOM | 811 | H | GLN | A | 89 | 101.576 | 207.806 | −29.319 | 0.00 | 0.00 | AAAA | H |
| ATOM | 812 | 1HE2 | GLN | A | 89 | 105.442 | 212.600 | −30.178 | 0.00 | 0.00 | AAAA | H |
| ATOM | 813 | 2HE2 | GLN | A | 89 | 105.827 | 211.489 | −28.910 | 0.00 | 0.00 | AAAA | H |
| ATOM | 814 | N | LYS | A | 90 | 99.736 | 208.974 | −27.070 | 1.00 | 50.00 | AAAA | N |
| ATOM | 815 | CA | LYS | A | 90 | 98.559 | 209.097 | −26.216 | 1.00 | 50.00 | AAAA | C |
| ATOM | 816 | C | LYS | A | 90 | 98.462 | 208.007 | −25.173 | 1.00 | 50.00 | AAAA | C |
| ATOM | 817 | O | LYS | A | 90 | 98.122 | 208.227 | −24.022 | 1.00 | 50.00 | AAAA | O |
| ATOM | 818 | CB | LYS | A | 90 | 97.302 | 209.098 | −27.076 | 1.00 | 50.00 | AAAA | C |
| ATOM | 819 | CG | LYS | A | 90 | 96.058 | 209.517 | −26.302 | 1.00 | 50.00 | AAAA | C |
| ATOM | 820 | CD | LYS | A | 90 | 96.320 | 210.856 | −25.621 | 1.00 | 50.00 | AAAA | C |
| ATOM | 821 | CE | LYS | A | 90 | 95.125 | 211.362 | −24.836 | 1.00 | 50.00 | AAAA | C |
| ATOM | 822 | NZ | LYS | A | 90 | 94.700 | 210.391 | −23.822 | 1.00 | 50.00 | AAAA | N |
| ATOM | 823 | H | LYS | A | 90 | 99.630 | 208.724 | −28.034 | 0.00 | 0.00 | AAAA | H |
| ATOM | 824 | 1HZ | LYS | A | 90 | 93.880 | 210.765 | −23.302 | 0.00 | 0.00 | AAAA | H |
| ATOM | 825 | 2HZ | LYS | A | 90 | 95.476 | 210.217 | −23.155 | 0.00 | 0.00 | AAAA | H |
| ATOM | 826 | 3HZ | LYS | A | 90 | 94.435 | 209.499 | −24.288 | 0.00 | 0.00 | AAAA | H |
| ATOM | 827 | N | ASN | A | 91 | 98.811 | 206.802 | −25.658 | 1.00 | 50.00 | AAAA | N |
| ATOM | 828 | CA | ASN | A | 91 | 98.831 | 205.609 | −24.817 | 1.00 | 50.00 | AAAA | C |
| ATOM | 829 | C | ASN | A | 91 | 99.761 | 205.720 | −23.620 | 1.00 | 50.00 | AAAA | C |
| ATOM | 830 | O | ASN | A | 91 | 99.445 | 205.279 | −22.528 | 1.00 | 50.00 | AAAA | O |
| ATOM | 831 | CB | ASN | A | 91 | 99.180 | 204.388 | −25.675 | 1.00 | 50.00 | AAAA | C |
| ATOM | 832 | CG | ASN | A | 91 | 99.079 | 203.117 | −24.864 | 1.00 | 50.00 | AAAA | C |
| ATOM | 833 | ND2 | ASN | A | 91 | 100.255 | 202.498 | −24.694 | 1.00 | 50.00 | AAAA | N |
| ATOM | 834 | OD1 | ASN | A | 91 | 98.017 | 202.734 | −24.396 | 1.00 | 50.00 | AAAA | O |
| ATOM | 835 | H | ASN | A | 91 | 99.060 | 206.741 | −26.624 | 0.00 | 0.00 | AAAA | H |
| ATOM | 836 | 1HD2 | ASN | A | 91 | 100.300 | 201.692 | −24.106 | 0.00 | 0.00 | AAAA | H |
| ATOM | 837 | 2HD2 | ASN | A | 91 | 101.089 | 202.823 | −25.139 | 0.00 | 0.00 | AAAA | H |
| ATOM | 838 | N | ASN | A | 92 | 100.922 | 206.342 | −23.884 | 1.00 | 50.00 | AAAA | N |
| ATOM | 839 | CA | ASN | A | 92 | 101.900 | 206.540 | −22.811 | 1.00 | 50.00 | AAAA | C |
| ATOM | 840 | C | ASN | A | 92 | 101.371 | 207.444 | −21.706 | 1.00 | 50.00 | AAAA | C |
| ATOM | 841 | O | ASN | A | 92 | 101.436 | 207.138 | −20.522 | 1.00 | 50.00 | AAAA | O |
| ATOM | 842 | CB | ASN | A | 92 | 103.206 | 207.077 | −23.410 | 1.00 | 50.00 | AAAA | C |
| ATOM | 843 | CG | ASN | A | 92 | 104.303 | 207.163 | −22.362 | 1.00 | 50.00 | AAAA | C |
| ATOM | 844 | ND2 | ASN | A | 92 | 105.373 | 206.406 | −22.649 | 1.00 | 50.00 | AAAA | N |
| ATOM | 845 | OD1 | ASN | A | 92 | 104.201 | 207.856 | −21.359 | 1.00 | 50.00 | AAAA | O |
| ATOM | 846 | H | ASN | A | 92 | 101.088 | 206.706 | −24.800 | 0.00 | 0.00 | AAAA | H |
| ATOM | 847 | 1HD2 | ASN | A | 92 | 106.130 | 206.393 | −21.996 | 0.00 | 0.00 | AAAA | H |
| ATOM | 848 | 2HD2 | ASN | A | 92 | 105.445 | 205.873 | −23.491 | 0.00 | 0.00 | AAAA | H |
| ATOM | 849 | N | ARG | A | 93 | 100.811 | 208.573 | −22.181 | 1.00 | 50.00 | AAAA | N |
| ATOM | 850 | CA | ARG | A | 93 | 100.177 | 209.517 | −21.256 | 1.00 | 50.00 | AAAA | C |
| ATOM | 851 | C | ARG | A | 93 | 99.005 | 208.936 | −20.480 | 1.00 | 50.00 | AAAA | C |
| ATOM | 852 | O | ARG | A | 93 | 98.796 | 209.176 | −19.297 | 1.00 | 50.00 | AAAA | O |
| ATOM | 853 | CB | ARG | A | 93 | 99.734 | 210.760 | −22.022 | 1.00 | 50.00 | AAAA | C |
| ATOM | 854 | CG | ARG | A | 93 | 100.917 | 211.551 | −22.579 | 1.00 | 50.00 | AAAA | C |
| ATOM | 855 | CD | ARG | A | 93 | 100.491 | 212.865 | −23.237 | 1.00 | 50.00 | AAAA | C |
| ATOM | 856 | NE | ARG | A | 93 | 99.817 | 213.756 | −22.288 | 1.00 | 50.00 | AAAA | N |
| ATOM | 857 | CZ | ARG | A | 93 | 100.511 | 214.656 | −21.561 | 1.00 | 50.00 | AAAA | C |
| ATOM | 858 | NH1 | ARG | A | 93 | 101.833 | 214.762 | −21.653 | 1.00 | 50.00 | AAAA | N |
| ATOM | 859 | NH2 | ARG | A | 93 | 99.865 | 215.459 | −20.729 | 1.00 | 50.00 | AAAA | N |
| ATOM | 860 | H | ARG | A | 93 | 100.803 | 208.738 | −23.169 | 0.00 | 0.00 | AAAA | H |
| ATOM | 861 | HE | ARG | A | 93 | 98.828 | 213.683 | −22.162 | 0.00 | 0.00 | AAAA | H |
| ATOM | 862 | 1HH1 | ARG | A | 93 | 102.320 | 215.439 | −21.103 | 0.00 | 0.00 | AAAA | H |
| ATOM | 863 | 2HH1 | ARG | A | 93 | 102.344 | 214.162 | −22.267 | 0.00 | 0.00 | AAAA | H |
| ATOM | 864 | 1HH2 | ARG | A | 93 | 100.376 | 216.122 | −20.185 | 0.00 | 0.00 | AAAA | H |
| ATOM | 865 | 2HH2 | ARG | A | 93 | 98.871 | 215.401 | −20.638 | 0.00 | 0.00 | AAAA | H |
| ATOM | 866 | N | LEU | A | 94 | 98.257 | 208.113 | −21.225 | 1.00 | 50.00 | AAAA | N |
| ATOM | 867 | CA | LEU | A | 94 | 97.108 | 207.423 | −20.652 | 1.00 | 50.00 | AAAA | C |
| ATOM | 868 | C | LEU | A | 94 | 97.468 | 206.452 | −19.544 | 1.00 | 50.00 | AAAA | C |
| ATOM | 869 | O | LEU | A | 94 | 96.799 | 206.335 | −18.528 | 1.00 | 50.00 | AAAA | O |
| ATOM | 870 | CB | LEU | A | 94 | 96.363 | 206.716 | −21.779 | 1.00 | 50.00 | AAAA | C |
| ATOM | 871 | CG | LEU | A | 94 | 94.851 | 206.849 | −21.664 | 1.00 | 50.00 | AAAA | C |
| ATOM | 872 | CD1 | LEU | A | 94 | 94.455 | 208.302 | −21.427 | 1.00 | 50.00 | AAAA | C |
| ATOM | 873 | CD2 | LEU | A | 94 | 94.146 | 206.240 | −22.876 | 1.00 | 50.00 | AAAA | C |
| ATOM | 874 | H | LEU | A | 94 | 98.525 | 207.973 | −22.174 | 0.00 | 0.00 | AAAA | H |
| ATOM | 875 | N | LEU | A | 95 | 98.612 | 205.793 | −19.806 | 1.00 | 50.00 | AAAA | N |
| ATOM | 876 | CA | LEU | A | 95 | 99.220 | 204.844 | −18.874 | 1.00 | 50.00 | AAAA | C |
| ATOM | 877 | C | LEU | A | 95 | 99.783 | 205.499 | −17.614 | 1.00 | 50.00 | AAAA | C |
| ATOM | 878 | O | LEU | A | 95 | 100.047 | 204.858 | −16.604 | 1.00 | 50.00 | AAAA | O |
| ATOM | 879 | CB | LEU | A | 95 | 100.270 | 204.041 | −19.659 | 1.00 | 50.00 | AAAA | C |
| ATOM | 880 | CG | LEU | A | 95 | 100.914 | 202.820 | −18.991 | 1.00 | 50.00 | AAAA | C |
| ATOM | 881 | CD1 | LEU | A | 95 | 101.185 | 201.714 | −20.013 | 1.00 | 50.00 | AAAA | C |
| ATOM | 882 | CD2 | LEU | A | 95 | 102.196 | 203.158 | −18.224 | 1.00 | 50.00 | AAAA | C |
| ATOM | 883 | H | LEU | A | 95 | 99.081 | 205.980 | −20.668 | 0.00 | 0.00 | AAAA | H |
| ATOM | 884 | N | GLU | A | 96 | 99.934 | 206.831 | −17.714 | 1.00 | 50.00 | AAAA | N |
| ATOM | 885 | CA | GLU | A | 96 | 100.359 | 207.602 | −16.551 | 1.00 | 50.00 | AAAA | C |
| ATOM | 886 | C | GLU | A | 96 | 99.231 | 207.857 | −15.553 | 1.00 | 50.00 | AAAA | C |
| ATOM | 887 | O | GLU | A | 96 | 99.451 | 208.102 | −14.373 | 1.00 | 50.00 | AAAA | O |
| ATOM | 888 | CB | GLU | A | 96 | 101.035 | 208.879 | −17.073 | 1.00 | 50.00 | AAAA | C |
| ATOM | 889 | CG | GLU | A | 96 | 101.698 | 209.843 | −16.077 | 1.00 | 50.00 | AAAA | C |
| ATOM | 890 | CD | GLU | A | 96 | 100.693 | 210.619 | −15.233 | 1.00 | 50.00 | AAAA | C |
| ATOM | 891 | OE1 | GLU | A | 96 | 99.574 | 210.867 | −15.679 | 1.00 | 50.00 | AAAA | O |
| ATOM | 892 | OE2 | GLU | A | 96 | 101.048 | 210.975 | −14.112 | 1.00 | 50.00 | AAAA | O |
| ATOM | 893 | H | GLU | A | 96 | 99.660 | 207.316 | −18.544 | 0.00 | 0.00 | AAAA | H |
| ATOM | 894 | N | ILE | A | 97 | 97.996 | 207.775 | −16.083 | 1.00 | 50.00 | AAAA | N |
| ATOM | 895 | CA | ILE | A | 97 | 96.836 | 208.082 | −15.242 | 1.00 | 50.00 | AAAA | C |
| ATOM | 896 | C | ILE | A | 97 | 96.262 | 206.852 | −14.563 | 1.00 | 50.00 | AAAA | C |
| ATOM | 897 | O | ILE | A | 97 | 95.858 | 206.840 | −13.404 | 1.00 | 50.00 | AAAA | O |
| ATOM | 898 | CB | ILE | A | 97 | 95.767 | 208.746 | −16.120 | 1.00 | 50.00 | AAAA | C |
| ATOM | 899 | CG1 | ILE | A | 97 | 96.242 | 210.114 | −16.587 | 1.00 | 50.00 | AAAA | C |
| ATOM | 900 | CG2 | ILE | A | 97 | 94.418 | 208.876 | −15.401 | 1.00 | 50.00 | AAAA | C |
| ATOM | 901 | CD1 | ILE | A | 97 | 95.260 | 210.703 | −17.596 | 1.00 | 50.00 | AAAA | C |
| ATOM | 902 | H | ILE | A | 97 | 97.865 | 207.469 | −17.026 | 0.00 | 0.00 | AAAA | H |
| ATOM | 903 | N | THR | A | 98 | 96.205 | 205.816 | −15.399 | 1.00 | 50.00 | AAAA | N |
| ATOM | 904 | CA | THR | A | 98 | 95.346 | 204.705 | −15.056 | 1.00 | 50.00 | AAAA | C |
| ATOM | 905 | C | THR | A | 98 | 96.126 | 203.493 | −14.592 | 1.00 | 50.00 | AAAA | C |
| ATOM | 906 | O | THR | A | 98 | 96.255 | 202.474 | −15.263 | 1.00 | 50.00 | AAAA | O |
| ATOM | 907 | CB | THR | A | 98 | 94.498 | 204.435 | −16.279 | 1.00 | 50.00 | AAAA | C |
| ATOM | 908 | CG2 | THR | A | 98 | 93.363 | 205.443 | −16.470 | 1.00 | 50.00 | AAAA | C |
| ATOM | 909 | OG1 | THR | A | 98 | 95.348 | 204.441 | −17.428 | 1.00 | 50.00 | AAAA | O |
| ATOM | 910 | H | THR | A | 98 | 96.620 | 205.837 | −16.310 | 0.00 | 0.00 | AAAA | H |
| ATOM | 911 | HG1 | THR | A | 98 | 94.784 | 204.279 | −18.169 | 0.00 | 0.00 | AAAA | H |
| ATOM | 912 | N | ARG | A | 99 | 96.644 | 203.682 | −13.369 | 1.00 | 50.00 | AAAA | N |
| ATOM | 913 | CA | ARG | A | 99 | 97.359 | 202.586 | −12.735 | 1.00 | 50.00 | AAAA | C |
| ATOM | 914 | C | ARG | A | 99 | 96.503 | 201.949 | −11.665 | 1.00 | 50.00 | AAAA | C |
| ATOM | 915 | O | ARG | A | 99 | 96.049 | 202.580 | −10.716 | 1.00 | 50.00 | AAAA | O |
| ATOM | 916 | CB | ARG | A | 99 | 98.691 | 203.066 | −12.158 | 1.00 | 50.00 | AAAA | C |
| ATOM | 917 | CG | ARG | A | 99 | 99.530 | 201.922 | −11.581 | 1.00 | 50.00 | AAAA | C |
| ATOM | 918 | CD | ARG | A | 99 | 100.881 | 202.391 | −11.042 | 1.00 | 50.00 | AAAA | C |
| ATOM | 919 | NE | ARG | A | 99 | 101.672 | 203.035 | −12.093 | 1.00 | 50.00 | AAAA | N |
| ATOM | 920 | CZ | ARG | A | 99 | 102.514 | 202.315 | −12.865 | 1.00 | 50.00 | AAAA | C |
| ATOM | 921 | NH1 | ARG | A | 99 | 102.651 | 201.000 | −12.704 | 1.00 | 50.00 | AAAA | N |
| ATOM | 922 | NH2 | ARG | A | 99 | 103.219 | 202.929 | −13.808 | 1.00 | 50.00 | AAAA | N |
| ATOM | 923 | H | ARG | A | 99 | 96.471 | 204.523 | −12.854 | 0.00 | 0.00 | AAAA | H |
| ATOM | 924 | HE | ARG | A | 99 | 101.551 | 204.012 | −12.261 | 0.00 | 0.00 | AAAA | H |
| ATOM | 925 | 1HH1 | ARG | A | 99 | 103.288 | 200.486 | −13.276 | 0.00 | 0.00 | AAAA | H |
| ATOM | 926 | 2HH1 | ARG | A | 99 | 102.117 | 200.523 | −12.005 | 0.00 | 0.00 | AAAA | H |
| ATOM | 927 | 1HH2 | ARG | A | 99 | 103.850 | 202.408 | −14.383 | 0.00 | 0.00 | AAAA | H |
| ATOM | 928 | 2HH2 | ARG | A | 99 | 103.122 | 203.915 | −13.947 | 0.00 | 0.00 | AAAA | H |
| ATOM | 929 | N | GLU | A | 100 | 96.290 | 200.642 | −11.877 | 1.00 | 50.00 | AAAA | N |
| ATOM | 930 | CA | GLU | A | 100 | 95.559 | 199.953 | −10.828 | 1.00 | 50.00 | AAAA | C |
| ATOM | 931 | C | GLU | A | 100 | 96.369 | 199.661 | −9.582 | 1.00 | 50.00 | AAAA | C |
| ATOM | 932 | O | GLU | A | 100 | 97.343 | 198.918 | −9.571 | 1.00 | 50.00 | AAAA | O |
| ATOM | 933 | CB | GLU | A | 100 | 94.892 | 198.696 | −11.356 | 1.00 | 50.00 | AAAA | C |
| ATOM | 934 | CG | GLU | A | 100 | 93.973 | 198.081 | −10.292 | 1.00 | 50.00 | AAAA | C |
| ATOM | 935 | CD | GLU | A | 100 | 94.662 | 197.018 | −9.444 | 1.00 | 50.00 | AAAA | C |
| ATOM | 936 | OE1 | GLU | A | 100 | 95.652 | 196.427 | −9.881 | 1.00 | 50.00 | AAAA | O |
| ATOM | 937 | OE2 | GLU | A | 100 | 94.195 | 196.781 | −8.331 | 1.00 | 50.00 | AAAA | O |
| ATOM | 938 | H | GLU | A | 100 | 96.673 | 200.174 | −12.672 | 0.00 | 0.00 | AAAA | H |
| ATOM | 939 | N | PHE | A | 101 | 95.860 | 200.288 | −8.515 | 1.00 | 50.00 | AAAA | N |
| ATOM | 940 | CA | PHE | A | 101 | 96.498 | 200.119 | −7.217 | 1.00 | 50.00 | AAAA | C |
| ATOM | 941 | C | PHE | A | 101 | 95.487 | 199.949 | −6.089 | 1.00 | 50.00 | AAAA | C |
| ATOM | 942 | O | PHE | A | 101 | 95.711 | 199.248 | −5.110 | 1.00 | 50.00 | AAAA | O |
| ATOM | 943 | CB | PHE | A | 101 | 97.435 | 201.315 | −7.012 | 1.00 | 50.00 | AAAA | C |
| ATOM | 944 | CG | PHE | A | 101 | 98.279 | 201.141 | −5.773 | 1.00 | 50.00 | AAAA | C |
| ATOM | 945 | CD1 | PHE | A | 101 | 99.421 | 200.313 | −5.811 | 1.00 | 50.00 | AAAA | C |
| ATOM | 946 | CD2 | PHE | A | 101 | 97.900 | 201.817 | −4.593 | 1.00 | 50.00 | AAAA | C |
| ATOM | 947 | CE1 | PHE | A | 101 | 100.199 | 200.157 | −4.648 | 1.00 | 50.00 | AAAA | C |
| ATOM | 948 | CE2 | PHE | A | 101 | 98.676 | 201.661 | −3.430 | 1.00 | 50.00 | AAAA | C |
| ATOM | 949 | CZ | PHE | A | 101 | 99.816 | 200.832 | −3.468 | 1.00 | 50.00 | AAAA | C |
| ATOM | 950 | H | PHE | A | 101 | 95.121 | 200.948 | −8.645 | 0.00 | 0.00 | AAAA | H |
| ATOM | 951 | N | SER | A | 102 | 94.343 | 200.632 | −6.294 | 1.00 | 50.00 | AAAA | N |
| ATOM | 952 | CA | SER | A | 102 | 93.279 | 200.547 | −5.302 | 1.00 | 50.00 | AAAA | C |
| ATOM | 953 | C | SER | A | 102 | 92.326 | 199.392 | −5.576 | 1.00 | 50.00 | AAAA | C |
| ATOM | 954 | O | SER | A | 102 | 92.323 | 198.797 | −6.647 | 1.00 | 50.00 | AAAA | O |
| ATOM | 955 | CB | SER | A | 102 | 92.541 | 201.896 | −5.265 | 1.00 | 50.00 | AAAA | C |
| ATOM | 956 | OG | SER | A | 102 | 91.529 | 201.917 | −4.250 | 1.00 | 50.00 | AAAA | O |
| ATOM | 957 | H | SER | A | 102 | 94.204 | 201.155 | −7.130 | 0.00 | 0.00 | AAAA | H |
| ATOM | 958 | HG | SER | A | 102 | 91.124 | 202.776 | −4.291 | 0.00 | 0.00 | AAAA | H |
| ATOM | 959 | N | VAL | A | 103 | 91.492 | 199.133 | −4.544 | 1.00 | 99.99 | AAAA | N |
| ATOM | 960 | CA | VAL | A | 103 | 90.414 | 198.148 | −4.671 | 1.00 | 99.99 | AAAA | C |
| ATOM | 961 | C | VAL | A | 103 | 89.321 | 198.543 | −5.670 | 1.00 | 99.99 | AAAA | C |
| ATOM | 962 | O | VAL | A | 103 | 88.620 | 197.715 | −6.237 | 1.00 | 99.99 | AAAA | O |
| ATOM | 963 | CB | VAL | A | 103 | 89.846 | 197.851 | −3.263 | 1.00 | 99.99 | AAAA | C |
| ATOM | 964 | CG1 | VAL | A | 103 | 89.208 | 199.089 | −2.619 | 1.00 | 99.99 | AAAA | C |
| ATOM | 965 | CG2 | VAL | A | 103 | 88.916 | 196.636 | −3.236 | 1.00 | 99.99 | AAAA | C |
| ATOM | 966 | H | VAL | A | 103 | 91.612 | 199.669 | −3.708 | 0.00 | 0.00 | AAAA | H |
| ATOM | 967 | N | ASN | A | 104 | 89.217 | 199.872 | −5.860 | 1.00 | 99.99 | AAAA | N |
| ATOM | 968 | CA | ASN | A | 104 | 88.201 | 200.402 | −6.766 | 1.00 | 99.99 | AAAA | C |
| ATOM | 969 | C | ASN | A | 104 | 88.745 | 201.512 | −7.657 | 1.00 | 99.99 | AAAA | C |
| ATOM | 970 | O | ASN | A | 104 | 89.945 | 201.755 | −7.706 | 1.00 | 99.99 | AAAA | O |
| ATOM | 971 | CB | ASN | A | 104 | 86.960 | 200.840 | −5.960 | 1.00 | 99.99 | AAAA | C |
| ATOM | 972 | CG | ASN | A | 104 | 87.267 | 201.966 | −4.978 | 1.00 | 99.99 | AAAA | C |
| ATOM | 973 | ND2 | ASN | A | 104 | 86.502 | 201.928 | −3.873 | 1.00 | 99.99 | AAAA | N |
| ATOM | 974 | OD1 | ASN | A | 104 | 88.116 | 202.822 | −5.192 | 1.00 | 99.99 | AAAA | O |
| ATOM | 975 | H | ASN | A | 104 | 89.842 | 200.508 | −5.409 | 0.00 | 0.00 | AAAA | H |
| ATOM | 976 | 1HD2 | ASN | A | 104 | 86.614 | 202.650 | −3.191 | 0.00 | 0.00 | AAAA | H |
| ATOM | 977 | 2HD2 | ASN | A | 104 | 85.839 | 201.196 | −3.720 | 0.00 | 0.00 | AAAA | H |
| ATOM | 978 | N | ALA | A | 105 | 87.800 | 202.197 | −8.334 | 1.00 | 99.99 | AAAA | N |
| ATOM | 979 | CA | ALA | A | 105 | 88.188 | 203.425 | −9.023 | 1.00 | 99.99 | AAAA | C |
| ATOM | 980 | C | ALA | A | 105 | 87.703 | 204.676 | −8.312 | 1.00 | 99.99 | AAAA | C |
| ATOM | 981 | O | ALA | A | 105 | 86.632 | 204.711 | −7.716 | 1.00 | 99.99 | AAAA | O |
| ATOM | 982 | CB | ALA | A | 105 | 87.643 | 203.438 | −10.449 | 1.00 | 99.99 | AAAA | C |
| ATOM | 983 | H | ALA | A | 105 | 86.838 | 201.937 | −8.278 | 0.00 | 0.00 | AAAA | H |
| ATOM | 984 | N | GLY | A | 106 | 88.562 | 205.712 | −8.407 | 1.00 | 99.99 | AAAA | N |
| ATOM | 985 | CA | GLY | A | 106 | 88.271 | 206.965 | −7.705 | 1.00 | 99.99 | AAAA | C |
| ATOM | 986 | C | GLY | A | 106 | 87.687 | 208.055 | −8.585 | 1.00 | 99.99 | AAAA | C |
| ATOM | 987 | O | GLY | A | 106 | 86.738 | 207.825 | −9.329 | 1.00 | 99.99 | AAAA | O |
| ATOM | 988 | H | GLY | A | 106 | 89.383 | 205.617 | −8.968 | 0.00 | 0.00 | AAAA | H |
| ATOM | 989 | N | VAL | A | 107 | 88.314 | 209.252 | −8.459 | 1.00 | 99.99 | AAAA | N |
| ATOM | 990 | CA | VAL | A | 107 | 87.981 | 210.391 | −9.328 | 1.00 | 99.99 | AAAA | C |
| ATOM | 991 | C | VAL | A | 107 | 89.186 | 211.022 | −10.040 | 1.00 | 99.99 | AAAA | C |
| ATOM | 992 | O | VAL | A | 107 | 90.327 | 210.612 | −9.866 | 1.00 | 99.99 | AAAA | O |
| ATOM | 993 | CB | VAL | A | 107 | 87.117 | 211.433 | −8.595 | 1.00 | 99.99 | AAAA | C |
| ATOM | 994 | CG1 | VAL | A | 107 | 85.766 | 210.832 | −8.207 | 1.00 | 99.99 | AAAA | C |
| ATOM | 995 | CG2 | VAL | A | 107 | 87.828 | 212.053 | −7.394 | 1.00 | 99.99 | AAAA | C |
| ATOM | 996 | H | VAL | A | 107 | 89.042 | 209.366 | −7.784 | 0.00 | 0.00 | AAAA | H |
| ATOM | 997 | N | THR | A | 108 | 88.865 | 212.008 | −10.892 | 1.00 | 50.00 | AAAA | N |
| ATOM | 998 | CA | THR | A | 108 | 89.771 | 212.556 | −11.895 | 1.00 | 50.00 | AAAA | C |
| ATOM | 999 | C | THR | A | 108 | 90.016 | 214.036 | −11.628 | 1.00 | 50.00 | AAAA | C |
| ATOM | 1000 | O | THR | A | 108 | 89.103 | 214.835 | −11.449 | 1.00 | 50.00 | AAAA | O |
| ATOM | 1001 | CB | THR | A | 108 | 89.146 | 212.354 | −13.287 | 1.00 | 50.00 | AAAA | C |
| ATOM | 1002 | CG2 | THR | A | 108 | 90.069 | 212.645 | −14.462 | 1.00 | 50.00 | AAAA | C |
| ATOM | 1003 | OG1 | THR | A | 108 | 88.583 | 211.048 | −13.425 | 1.00 | 50.00 | AAAA | O |
| ATOM | 1004 | H | THR | A | 108 | 87.982 | 212.445 | −10.824 | 0.00 | 0.00 | AAAA | H |
| ATOM | 1005 | HG1 | THR | A | 108 | 88.206 | 211.009 | −14.299 | 0.00 | 0.00 | AAAA | H |
| ATOM | 1006 | N | THR | A | 109 | 91.318 | 214.345 | −11.593 | 1.00 | 50.00 | AAAA | N |
| ATOM | 1007 | CA | THR | A | 109 | 91.809 | 215.687 | −11.268 | 1.00 | 50.00 | AAAA | C |
| ATOM | 1008 | C | THR | A | 109 | 91.914 | 216.459 | −12.575 | 1.00 | 50.00 | AAAA | C |
| ATOM | 1009 | O | THR | A | 109 | 92.045 | 215.803 | −13.599 | 1.00 | 50.00 | AAAA | O |
| ATOM | 1010 | CB | THR | A | 109 | 93.202 | 215.466 | −10.650 | 1.00 | 50.00 | AAAA | C |
| ATOM | 1011 | CG2 | THR | A | 109 | 93.713 | 216.578 | −9.733 | 1.00 | 50.00 | AAAA | C |
| ATOM | 1012 | OG1 | THR | A | 109 | 93.226 | 214.217 | −9.956 | 1.00 | 50.00 | AAAA | O |
| ATOM | 1013 | H | THR | A | 109 | 91.965 | 213.619 | −11.829 | 0.00 | 0.00 | AAAA | H |
| ATOM | 1014 | HG1 | THR | A | 109 | 94.077 | 214.152 | −9.544 | 0.00 | 0.00 | AAAA | H |
| ATOM | 1015 | N | PRO | A | 110 | 91.910 | 217.830 | −12.558 | 1.00 | 50.00 | AAAA | N |
| ATOM | 1016 | CA | PRO | A | 110 | 92.249 | 218.581 | −13.783 | 1.00 | 50.00 | AAAA | C |
| ATOM | 1017 | C | PRO | A | 110 | 93.519 | 218.094 | −14.471 | 1.00 | 50.00 | AAAA | C |
| ATOM | 1018 | O | PRO | A | 110 | 93.677 | 218.167 | −15.683 | 1.00 | 50.00 | AAAA | O |
| ATOM | 1019 | CB | PRO | A | 110 | 92.359 | 220.033 | −13.303 | 1.00 | 50.00 | AAAA | C |
| ATOM | 1020 | CG | PRO | A | 110 | 92.488 | 219.966 | −11.785 | 1.00 | 50.00 | AAAA | C |
| ATOM | 1021 | CD | PRO | A | 110 | 91.666 | 218.731 | −11.438 | 1.00 | 50.00 | AAAA | C |
| ATOM | 1022 | N | VAL | A | 111 | 94.398 | 217.529 | −13.624 | 1.00 | 50.00 | AAAA | N |
| ATOM | 1023 | CA | VAL | A | 111 | 95.567 | 216.873 | −14.182 | 1.00 | 50.00 | AAAA | C |
| ATOM | 1024 | C | VAL | A | 111 | 95.302 | 215.671 | −15.078 | 1.00 | 50.00 | AAAA | C |
| ATOM | 1025 | O | VAL | A | 111 | 95.759 | 215.596 | −16.211 | 1.00 | 50.00 | AAAA | O |
| ATOM | 1026 | CB | VAL | A | 111 | 96.672 | 216.636 | −13.132 | 1.00 | 50.00 | AAAA | C |
| ATOM | 1027 | CG1 | VAL | A | 111 | 96.946 | 217.947 | −12.392 | 1.00 | 50.00 | AAAA | C |
| ATOM | 1028 | CG2 | VAL | A | 111 | 96.429 | 215.475 | −12.166 | 1.00 | 50.00 | AAAA | C |
| ATOM | 1029 | H | VAL | A | 111 | 94.232 | 217.611 | −12.644 | 0.00 | 0.00 | AAAA | H |
| ATOM | 1030 | N | SER | A | 112 | 94.475 | 214.760 | −14.559 | 1.00 | 50.00 | AAAA | N |
| ATOM | 1031 | CA | SER | A | 112 | 94.107 | 213.640 | −15.410 | 1.00 | 50.00 | AAAA | C |
| ATOM | 1032 | C | SER | A | 112 | 93.241 | 214.040 | −16.605 | 1.00 | 50.00 | AAAA | C |
| ATOM | 1033 | O | SER | A | 112 | 93.339 | 213.464 | −17.680 | 1.00 | 50.00 | AAAA | O |
| ATOM | 1034 | CB | SER | A | 112 | 93.498 | 212.558 | −14.516 | 1.00 | 50.00 | AAAA | C |
| ATOM | 1035 | OG | SER | A | 112 | 92.929 | 211.506 | −15.298 | 1.00 | 50.00 | AAAA | O |
| ATOM | 1036 | H | SER | A | 112 | 94.063 | 214.858 | −13.655 | 0.00 | 0.00 | AAAA | H |
| ATOM | 1037 | HG | SER | A | 112 | 92.751 | 210.790 | −14.701 | 0.00 | 0.00 | AAAA | H |
| ATOM | 1038 | N | THR | A | 113 | 92.434 | 215.101 | −16.379 | 1.00 | 50.00 | AAAA | N |
| ATOM | 1039 | CA | THR | A | 113 | 91.563 | 215.618 | −17.439 | 1.00 | 50.00 | AAAA | C |
| ATOM | 1040 | C | THR | A | 113 | 92.300 | 216.176 | −18.643 | 1.00 | 50.00 | AAAA | C |
| ATOM | 1041 | O | THR | A | 113 | 91.858 | 216.023 | −19.773 | 1.00 | 50.00 | AAAA | O |
| ATOM | 1042 | CB | THR | A | 113 | 90.562 | 216.658 | −16.903 | 1.00 | 50.00 | AAAA | C |
| ATOM | 1043 | CG2 | THR | A | 113 | 89.865 | 216.167 | −15.640 | 1.00 | 50.00 | AAAA | C |
| ATOM | 1044 | OG1 | THR | A | 113 | 91.190 | 217.910 | −16.618 | 1.00 | 50.00 | AAAA | O |
| ATOM | 1045 | H | THR | A | 113 | 92.444 | 215.517 | −15.473 | 0.00 | 0.00 | AAAA | H |
| ATOM | 1046 | HG1 | THR | A | 113 | 90.567 | 218.428 | −16.124 | 0.00 | 0.00 | AAAA | H |
| ATOM | 1047 | N | TYR | A | 114 | 93.464 | 216.809 | −18.344 | 1.00 | 50.00 | AAAA | N |
| ATOM | 1048 | CA | TYR | A | 114 | 94.236 | 217.392 | −19.444 | 1.00 | 50.00 | AAAA | C |
| ATOM | 1049 | C | TYR | A | 114 | 94.870 | 216.384 | −20.385 | 1.00 | 50.00 | AAAA | C |
| ATOM | 1050 | O | TYR | A | 114 | 94.928 | 216.595 | −21.589 | 1.00 | 50.00 | AAAA | O |
| ATOM | 1051 | CB | TYR | A | 114 | 95.236 | 218.502 | −19.004 | 1.00 | 50.00 | AAAA | C |
| ATOM | 1052 | CG | TYR | A | 114 | 96.479 | 218.042 | −18.250 | 1.00 | 50.00 | AAAA | C |
| ATOM | 1053 | CD1 | TYR | A | 114 | 97.459 | 217.249 | −18.886 | 1.00 | 50.00 | AAAA | C |
| ATOM | 1054 | CD2 | TYR | A | 114 | 96.640 | 218.434 | −16.907 | 1.00 | 50.00 | AAAA | C |
| ATOM | 1055 | CE1 | TYR | A | 114 | 98.498 | 216.690 | −18.123 | 1.00 | 50.00 | AAAA | C |
| ATOM | 1056 | CE2 | TYR | A | 114 | 97.696 | 217.886 | −16.153 | 1.00 | 50.00 | AAAA | C |
| ATOM | 1057 | CZ | TYR | A | 114 | 98.535 | 216.923 | −16.739 | 1.00 | 50.00 | AAAA | C |
| ATOM | 1058 | OH | TYR | A | 114 | 99.373 | 216.161 | −15.946 | 1.00 | 50.00 | AAAA | O |
| ATOM | 1059 | H | TYR | A | 114 | 93.740 | 216.930 | −17.391 | 0.00 | 0.00 | AAAA | H |
| ATOM | 1060 | HH | TYR | A | 114 | 98.958 | 215.978 | −15.111 | 0.00 | 0.00 | AAAA | H |
| ATOM | 1061 | N | MET | A | 115 | 95.319 | 215.267 | −19.775 | 1.00 | 50.00 | AAAA | N |
| ATOM | 1062 | CA | MET | A | 115 | 95.849 | 214.193 | −20.620 | 1.00 | 50.00 | AAAA | C |
| ATOM | 1063 | C | MET | A | 115 | 94.751 | 213.521 | −21.397 | 1.00 | 50.00 | AAAA | C |
| ATOM | 1064 | O | MET | A | 115 | 94.862 | 213.232 | −22.578 | 1.00 | 50.00 | AAAA | O |
| ATOM | 1065 | CB | MET | A | 115 | 96.592 | 213.130 | −19.813 | 1.00 | 50.00 | AAAA | C |
| ATOM | 1066 | CG | MET | A | 115 | 97.499 | 213.763 | −18.771 | 1.00 | 50.00 | AAAA | C |
| ATOM | 1067 | SD | MET | A | 115 | 98.333 | 212.670 | −17.628 | 1.00 | 50.00 | AAAA | S |
| ATOM | 1068 | CE | MET | A | 115 | 99.695 | 212.212 | −18.697 | 1.00 | 50.00 | AAAA | C |
| ATOM | 1069 | H | MET | A | 115 | 95.265 | 215.166 | −18.782 | 0.00 | 0.00 | AAAA | H |
| ATOM | 1070 | N | LEU | A | 116 | 93.652 | 213.323 | −20.652 | 1.00 | 50.00 | AAAA | N |
| ATOM | 1071 | CA | LEU | A | 116 | 92.511 | 212.605 | −21.194 | 1.00 | 50.00 | AAAA | C |
| ATOM | 1072 | C | LEU | A | 116 | 91.904 | 213.285 | −22.409 | 1.00 | 50.00 | AAAA | C |
| ATOM | 1073 | O | LEU | A | 116 | 91.567 | 212.669 | −23.411 | 1.00 | 50.00 | AAAA | O |
| ATOM | 1074 | CB | LEU | A | 116 | 91.488 | 212.445 | −20.076 | 1.00 | 50.00 | AAAA | C |
| ATOM | 1075 | CG | LEU | A | 116 | 90.696 | 211.152 | −20.138 | 1.00 | 50.00 | AAAA | C |
| ATOM | 1076 | CD1 | LEU | A | 116 | 91.593 | 209.936 | −20.339 | 1.00 | 50.00 | AAAA | C |
| ATOM | 1077 | CD2 | LEU | A | 116 | 89.831 | 210.994 | −18.889 | 1.00 | 50.00 | AAAA | C |
| ATOM | 1078 | H | LEU | A | 116 | 93.656 | 213.608 | −19.694 | 0.00 | 0.00 | AAAA | H |
| ATOM | 1079 | N | THR | A | 117 | 91.807 | 214.616 | −22.269 | 1.00 | 50.00 | AAAA | N |
| ATOM | 1080 | CA | THR | A | 117 | 91.132 | 215.393 | −23.303 | 1.00 | 50.00 | AAAA | C |
| ATOM | 1081 | C | THR | A | 117 | 92.033 | 216.043 | −24.346 | 1.00 | 50.00 | AAAA | C |
| ATOM | 1082 | O | THR | A | 117 | 91.624 | 216.240 | −25.481 | 1.00 | 50.00 | AAAA | O |
| ATOM | 1083 | CB | THR | A | 117 | 90.182 | 216.425 | −22.681 | 1.00 | 50.00 | AAAA | C |
| ATOM | 1084 | CG2 | THR | A | 117 | 89.104 | 215.768 | −21.814 | 1.00 | 50.00 | AAAA | C |
| ATOM | 1085 | OG1 | THR | A | 117 | 90.894 | 217.416 | −21.936 | 1.00 | 50.00 | AAAA | O |
| ATOM | 1086 | H | THR | A | 117 | 92.182 | 215.080 | −21.466 | 0.00 | 0.00 | AAAA | H |
| ATOM | 1087 | HG1 | THR | A | 117 | 90.247 | 217.950 | −21.494 | 0.00 | 0.00 | AAAA | H |
| ATOM | 1088 | N | ASN | A | 118 | 93.283 | 216.351 | −23.924 | 1.00 | 50.00 | AAAA | N |
| ATOM | 1089 | CA | ASN | A | 118 | 94.252 | 217.065 | −24.777 | 1.00 | 50.00 | AAAA | C |
| ATOM | 1090 | C | ASN | A | 118 | 93.797 | 218.502 | −25.111 | 1.00 | 50.00 | AAAA | C |
| ATOM | 1091 | O | ASN | A | 118 | 93.266 | 219.191 | −24.248 | 1.00 | 50.00 | AAAA | O |
| ATOM | 1092 | CB | ASN | A | 118 | 94.646 | 216.142 | −25.956 | 1.00 | 50.00 | AAAA | C |
| ATOM | 1093 | CG | ASN | A | 118 | 95.749 | 216.671 | −26.853 | 1.00 | 50.00 | AAAA | C |
| ATOM | 1094 | ND2 | ASN | A | 118 | 95.398 | 216.664 | −28.153 | 1.00 | 50.00 | AAAA | N |
| ATOM | 1095 | OD1 | ASN | A | 118 | 96.808 | 217.098 | −26.418 | 1.00 | 50.00 | AAAA | O |
| ATOM | 1096 | H | ASN | A | 118 | 93.505 | 216.128 | −22.973 | 0.00 | 0.00 | AAAA | H |
| ATOM | 1097 | 1HD2 | ASN | A | 118 | 96.008 | 217.028 | −28.855 | 0.00 | 0.00 | AAAA | H |
| ATOM | 1098 | 2HD2 | ASN | A | 118 | 94.505 | 216.315 | −28.438 | 0.00 | 0.00 | AAAA | H |
| ATOM | 1099 | N | SER | A | 119 | 93.994 | 218.922 | −26.387 | 1.00 | 50.00 | AAAA | N |
| ATOM | 1100 | CA | SER | A | 119 | 93.649 | 220.258 | −26.883 | 1.00 | 50.00 | AAAA | C |
| ATOM | 1101 | C | SER | A | 119 | 92.165 | 220.585 | −26.847 | 1.00 | 50.00 | AAAA | C |
| ATOM | 1102 | O | SER | A | 119 | 91.711 | 221.709 | −27.031 | 1.00 | 50.00 | AAAA | O |
| ATOM | 1103 | CB | SER | A | 119 | 94.174 | 220.381 | −28.321 | 1.00 | 50.00 | AAAA | C |
| ATOM | 1104 | OG | SER | A | 119 | 93.648 | 219.319 | −29.135 | 1.00 | 50.00 | AAAA | O |
| ATOM | 1105 | H | SER | A | 119 | 94.338 | 218.316 | −27.096 | 0.00 | 0.00 | AAAA | H |
| ATOM | 1106 | HG | SER | A | 119 | 93.994 | 219.439 | −30.012 | 0.00 | 0.00 | AAAA | H |
| ATOM | 1107 | N | GLU | A | 120 | 91.422 | 219.502 | −26.595 | 1.00 | 50.00 | AAAA | N |
| ATOM | 1108 | CA | GLU | A | 120 | 89.982 | 219.584 | −26.500 | 1.00 | 50.00 | AAAA | C |
| ATOM | 1109 | C | GLU | A | 120 | 89.515 | 220.285 | −25.227 | 1.00 | 50.00 | AAAA | C |
| ATOM | 1110 | O | GLU | A | 120 | 88.429 | 220.848 | −25.182 | 1.00 | 50.00 | AAAA | O |
| ATOM | 1111 | CB | GLU | A | 120 | 89.524 | 218.134 | −26.654 | 1.00 | 50.00 | AAAA | C |
| ATOM | 1112 | CG | GLU | A | 120 | 88.036 | 217.853 | −26.758 | 1.00 | 50.00 | AAAA | C |
| ATOM | 1113 | CD | GLU | A | 120 | 87.331 | 218.214 | −25.476 | 1.00 | 50.00 | AAAA | C |
| ATOM | 1114 | OE1 | GLU | A | 120 | 87.865 | 217.923 | −24.402 | 1.00 | 50.00 | AAAA | O |
| ATOM | 1115 | OE2 | GLU | A | 120 | 86.251 | 218.790 | −25.558 | 1.00 | 50.00 | AAAA | O |
| ATOM | 1116 | H | GLU | A | 120 | 91.851 | 218.623 | −26.388 | 0.00 | 0.00 | AAAA | H |
| ATOM | 1117 | N | LEU | A | 121 | 90.391 | 220.232 | −24.195 | 1.00 | 50.00 | AAAA | N |
| ATOM | 1118 | CA | LEU | A | 121 | 90.003 | 220.697 | −22.856 | 1.00 | 50.00 | AAAA | C |
| ATOM | 1119 | C | LEU | A | 121 | 89.343 | 222.061 | −22.797 | 1.00 | 50.00 | AAAA | C |
| ATOM | 1120 | O | LEU | A | 121 | 88.275 | 222.225 | −22.230 | 1.00 | 50.00 | AAAA | O |
| ATOM | 1121 | CB | LEU | A | 121 | 91.206 | 220.663 | −21.903 | 1.00 | 50.00 | AAAA | C |
| ATOM | 1122 | CG | LEU | A | 121 | 90.864 | 220.955 | −20.434 | 1.00 | 50.00 | AAAA | C |
| ATOM | 1123 | CD1 | LEU | A | 121 | 89.846 | 219.967 | −19.856 | 1.00 | 50.00 | AAAA | C |
| ATOM | 1124 | CD2 | LEU | A | 121 | 92.118 | 221.070 | −19.570 | 1.00 | 50.00 | AAAA | C |
| ATOM | 1125 | H | LEU | A | 121 | 91.274 | 219.779 | −24.321 | 0.00 | 0.00 | AAAA | H |
| ATOM | 1126 | N | LEU | A | 122 | 90.036 | 223.026 | −23.435 | 1.00 | 50.00 | AAAA | N |
| ATOM | 1127 | CA | LEU | A | 122 | 89.505 | 224.388 | −23.490 | 1.00 | 50.00 | AAAA | C |
| ATOM | 1128 | C | LEU | A | 122 | 88.089 | 224.493 | −24.049 | 1.00 | 50.00 | AAAA | C |
| ATOM | 1129 | O | LEU | A | 122 | 87.202 | 225.110 | −23.471 | 1.00 | 50.00 | AAAA | O |
| ATOM | 1130 | CB | LEU | A | 122 | 90.462 | 225.272 | −24.292 | 1.00 | 50.00 | AAAA | C |
| ATOM | 1131 | CG | LEU | A | 122 | 91.835 | 225.524 | −23.657 | 1.00 | 50.00 | AAAA | C |
| ATOM | 1132 | CD1 | LEU | A | 122 | 92.690 | 226.405 | −24.567 | 1.00 | 50.00 | AAAA | C |
| ATOM | 1133 | CD2 | LEU | A | 122 | 91.765 | 226.083 | −22.233 | 1.00 | 50.00 | AAAA | C |
| ATOM | 1134 | H | LEU | A | 122 | 90.921 | 222.810 | −23.845 | 0.00 | 0.00 | AAAA | H |
| ATOM | 1135 | N | SER | A | 123 | 87.918 | 223.812 | −25.194 | 1.00 | 50.00 | AAAA | N |
| ATOM | 1136 | CA | SER | A | 123 | 86.609 | 223.805 | −25.846 | 1.00 | 50.00 | AAAA | C |
| ATOM | 1137 | C | SER | A | 123 | 85.488 | 223.284 | −24.956 | 1.00 | 50.00 | AAAA | C |
| ATOM | 1138 | O | SER | A | 123 | 84.440 | 223.901 | −24.809 | 1.00 | 50.00 | AAAA | O |
| ATOM | 1139 | CB | SER | A | 123 | 86.715 | 223.005 | −27.151 | 1.00 | 50.00 | AAAA | C |
| ATOM | 1140 | OG | SER | A | 123 | 85.509 | 223.098 | −27.913 | 1.00 | 50.00 | AAAA | O |
| ATOM | 1141 | H | SER | A | 123 | 88.676 | 223.271 | −25.552 | 0.00 | 0.00 | AAAA | H |
| ATOM | 1142 | HG | SER | A | 123 | 85.589 | 222.484 | −28.631 | 0.00 | 0.00 | AAAA | H |
| ATOM | 1143 | N | LEU | A | 124 | 85.808 | 222.125 | −24.341 | 1.00 | 50.00 | AAAA | N |
| ATOM | 1144 | CA | LEU | A | 124 | 84.922 | 221.447 | −23.397 | 1.00 | 50.00 | AAAA | C |
| ATOM | 1145 | C | LEU | A | 124 | 84.467 | 222.338 | −22.266 | 1.00 | 50.00 | AAAA | C |
| ATOM | 1146 | O | LEU | A | 124 | 83.286 | 222.500 | −22.007 | 1.00 | 50.00 | AAAA | O |
| ATOM | 1147 | CB | LEU | A | 124 | 85.679 | 220.236 | −22.855 | 1.00 | 50.00 | AAAA | C |
| ATOM | 1148 | CG | LEU | A | 124 | 84.945 | 219.183 | −22.030 | 1.00 | 50.00 | AAAA | C |
| ATOM | 1149 | CD1 | LEU | A | 124 | 85.667 | 217.853 | −22.187 | 1.00 | 50.00 | AAAA | C |
| ATOM | 1150 | CD2 | LEU | A | 124 | 84.829 | 219.520 | −20.542 | 1.00 | 50.00 | AAAA | C |
| ATOM | 1151 | H | LEU | A | 124 | 86.721 | 221.748 | −24.489 | 0.00 | 0.00 | AAAA | H |
| ATOM | 1152 | N | ILE | A | 125 | 85.492 | 222.913 | −21.618 | 1.00 | 50.00 | AAAA | N |
| ATOM | 1153 | CA | ILE | A | 125 | 85.262 | 223.775 | −20.462 | 1.00 | 50.00 | AAAA | C |
| ATOM | 1154 | C | ILE | A | 125 | 84.376 | 224.972 | −20.758 | 1.00 | 50.00 | AAAA | C |
| ATOM | 1155 | O | ILE | A | 125 | 83.613 | 225.413 | −19.918 | 1.00 | 50.00 | AAAA | O |
| ATOM | 1156 | CB | ILE | A | 125 | 86.600 | 224.218 | −19.849 | 1.00 | 50.00 | AAAA | C |
| ATOM | 1157 | CG1 | ILE | A | 125 | 87.396 | 223.011 | −19.360 | 1.00 | 50.00 | AAAA | C |
| ATOM | 1158 | CG2 | ILE | A | 125 | 86.394 | 225.164 | −18.670 | 1.00 | 50.00 | AAAA | C |
| ATOM | 1159 | CD1 | ILE | A | 125 | 88.746 | 223.441 | −18.790 | 1.00 | 50.00 | AAAA | C |
| ATOM | 1160 | H | ILE | A | 125 | 86.411 | 222.727 | −21.958 | 0.00 | 0.00 | AAAA | H |
| ATOM | 1161 | N | ASN | A | 126 | 84.481 | 225.469 | −21.998 | 1.00 | 50.00 | AAAA | N |
| ATOM | 1162 | CA | ASN | A | 126 | 83.526 | 226.514 | −22.367 | 1.00 | 50.00 | AAAA | C |
| ATOM | 1163 | C | ASN | A | 126 | 82.115 | 226.013 | −22.584 | 1.00 | 50.00 | AAAA | C |
| ATOM | 1164 | O | ASN | A | 126 | 81.130 | 226.622 | −22.190 | 1.00 | 50.00 | AAAA | O |
| ATOM | 1165 | CB | ASN | A | 126 | 83.970 | 227.233 | −23.630 | 1.00 | 50.00 | AAAA | C |
| ATOM | 1166 | CG | ASN | A | 126 | 85.058 | 228.205 | −23.273 | 1.00 | 50.00 | AAAA | C |
| ATOM | 1167 | ND2 | ASN | A | 126 | 85.896 | 228.454 | −24.287 | 1.00 | 50.00 | AAAA | N |
| ATOM | 1168 | OD1 | ASN | A | 126 | 85.145 | 228.702 | −22.157 | 1.00 | 50.00 | AAAA | O |
| ATOM | 1169 | H | ASN | A | 126 | 85.184 | 225.144 | −22.628 | 0.00 | 0.00 | AAAA | H |
| ATOM | 1170 | 1HD2 | ASN | A | 126 | 86.669 | 229.060 | −24.111 | 0.00 | 0.00 | AAAA | H |
| ATOM | 1171 | 2HD2 | ASN | A | 126 | 85.787 | 228.028 | −25.184 | 0.00 | 0.00 | AAAA | H |
| ATOM | 1172 | N | ASP | A | 127 | 82.074 | 224.859 | −23.267 | 1.00 | 50.00 | AAAA | N |
| ATOM | 1173 | CA | ASP | A | 127 | 80.791 | 224.332 | −23.732 | 1.00 | 50.00 | AAAA | C |
| ATOM | 1174 | C | ASP | A | 127 | 79.904 | 223.734 | −22.646 | 1.00 | 50.00 | AAAA | C |
| ATOM | 1175 | O | ASP | A | 127 | 78.686 | 223.716 | −22.756 | 1.00 | 50.00 | AAAA | O |
| ATOM | 1176 | CB | ASP | A | 127 | 81.072 | 223.336 | −24.863 | 1.00 | 50.00 | AAAA | C |
| ATOM | 1177 | CG | ASP | A | 127 | 79.809 | 222.860 | −25.557 | 1.00 | 50.00 | AAAA | C |
| ATOM | 1178 | OD1 | ASP | A | 127 | 78.940 | 223.680 | −25.857 | 1.00 | 50.00 | AAAA | O |
| ATOM | 1179 | OD2 | ASP | A | 127 | 79.708 | 221.660 | −25.816 | 1.00 | 50.00 | AAAA | O |
| ATOM | 1180 | H | ASP | A | 127 | 82.918 | 224.368 | −23.485 | 0.00 | 0.00 | AAAA | H |
| ATOM | 1181 | N | MET | A | 128 | 80.568 | 223.227 | −21.596 | 1.00 | 50.00 | AAAA | N |
| ATOM | 1182 | CA | MET | A | 128 | 79.788 | 222.528 | −20.579 | 1.00 | 50.00 | AAAA | C |
| ATOM | 1183 | C | MET | A | 128 | 79.032 | 223.433 | −19.602 | 1.00 | 50.00 | AAAA | C |
| ATOM | 1184 | O | MET | A | 128 | 77.814 | 223.351 | −19.518 | 1.00 | 50.00 | AAAA | O |
| ATOM | 1185 | CB | MET | A | 128 | 80.625 | 221.406 | −19.932 | 1.00 | 50.00 | AAAA | C |
| ATOM | 1186 | CG | MET | A | 128 | 81.279 | 220.397 | −20.884 | 1.00 | 50.00 | AAAA | C |
| ATOM | 1187 | SD | MET | A | 128 | 80.205 | 219.146 | −21.610 | 1.00 | 50.00 | AAAA | S |
| ATOM | 1188 | CE | MET | A | 128 | 79.434 | 220.100 | −22.922 | 1.00 | 50.00 | AAAA | C |
| ATOM | 1189 | H | MET | A | 128 | 81.549 | 223.368 | −21.477 | 0.00 | 0.00 | AAAA | H |
| ATOM | 1190 | N | PRO | A | 129 | 79.760 | 224.329 | −18.880 | 1.00 | 50.00 | AAAA | N |
| ATOM | 1191 | CA | PRO | A | 129 | 79.051 | 225.358 | −18.119 | 1.00 | 50.00 | AAAA | C |
| ATOM | 1192 | C | PRO | A | 129 | 78.736 | 226.645 | −18.852 | 1.00 | 50.00 | AAAA | C |
| ATOM | 1193 | O | PRO | A | 129 | 79.275 | 226.979 | −19.899 | 1.00 | 50.00 | AAAA | O |
| ATOM | 1194 | CB | PRO | A | 129 | 80.049 | 225.689 | −17.040 | 1.00 | 50.00 | AAAA | C |
| ATOM | 1195 | CG | PRO | A | 129 | 81.417 | 225.535 | −17.678 | 1.00 | 50.00 | AAAA | C |
| ATOM | 1196 | CD | PRO | A | 129 | 81.198 | 224.373 | −18.626 | 1.00 | 50.00 | AAAA | C |
| ATOM | 1197 | N | ILE | A | 130 | 77.870 | 227.389 | −18.137 | 1.00 | 50.00 | AAAA | N |
| ATOM | 1198 | CA | ILE | A | 130 | 77.566 | 228.768 | −18.511 | 1.00 | 50.00 | AAAA | C |
| ATOM | 1199 | C | ILE | A | 130 | 78.034 | 229.818 | −17.501 | 1.00 | 50.00 | AAAA | C |
| ATOM | 1200 | O | ILE | A | 130 | 77.895 | 231.012 | −17.735 | 1.00 | 50.00 | AAAA | O |
| ATOM | 1201 | CB | ILE | A | 130 | 76.061 | 228.930 | −18.762 | 1.00 | 50.00 | AAAA | C |
| ATOM | 1202 | CG1 | ILE | A | 130 | 75.257 | 228.673 | −17.478 | 1.00 | 50.00 | AAAA | C |
| ATOM | 1203 | CG2 | ILE | A | 130 | 75.619 | 227.988 | −19.888 | 1.00 | 50.00 | AAAA | C |
| ATOM | 1204 | CD1 | ILE | A | 130 | 73.776 | 229.030 | −17.607 | 1.00 | 50.00 | AAAA | C |
| ATOM | 1205 | H | ILE | A | 130 | 77.451 | 226.999 | −17.319 | 0.00 | 0.00 | AAAA | H |
| ATOM | 1206 | N | THR | A | 131 | 78.580 | 229.328 | −16.360 | 1.00 | 50.00 | AAAA | N |
| ATOM | 1207 | CA | THR | A | 131 | 78.999 | 230.200 | −15.249 | 1.00 | 50.00 | AAAA | C |
| ATOM | 1208 | C | THR | A | 131 | 79.767 | 231.465 | −15.627 | 1.00 | 50.00 | AAAA | C |
| ATOM | 1209 | O | THR | A | 131 | 80.485 | 231.506 | −16.618 | 1.00 | 50.00 | AAAA | O |
| ATOM | 1210 | CB | THR | A | 131 | 79.809 | 229.356 | −14.253 | 1.00 | 50.00 | AAAA | C |
| ATOM | 1211 | CG2 | THR | A | 131 | 80.136 | 230.015 | −12.909 | 1.00 | 50.00 | AAAA | C |
| ATOM | 1212 | OG1 | THR | A | 131 | 79.098 | 228.157 | −13.991 | 1.00 | 50.00 | AAAA | O |
| ATOM | 1213 | H | THR | A | 131 | 78.598 | 228.340 | −16.208 | 0.00 | 0.00 | AAAA | H |
| ATOM | 1214 | HG1 | THR | A | 131 | 79.727 | 227.631 | −13.514 | 0.00 | 0.00 | AAAA | H |
| ATOM | 1215 | N | ASN | A | 132 | 79.593 | 232.495 | −14.764 | 1.00 | 50.00 | AAAA | N |
| ATOM | 1216 | CA | ASN | A | 132 | 80.324 | 233.757 | −14.942 | 1.00 | 50.00 | AAAA | C |
| ATOM | 1217 | C | ASN | A | 132 | 81.840 | 233.614 | −14.960 | 1.00 | 50.00 | AAAA | C |
| ATOM | 1218 | O | ASN | A | 132 | 82.557 | 234.360 | −15.614 | 1.00 | 50.00 | AAAA | O |
| ATOM | 1219 | CB | ASN | A | 132 | 79.943 | 234.773 | −13.860 | 1.00 | 50.00 | AAAA | C |
| ATOM | 1220 | CG | ASN | A | 132 | 78.454 | 235.051 | −13.865 | 1.00 | 50.00 | AAAA | C |
| ATOM | 1221 | ND2 | ASN | A | 132 | 78.035 | 235.738 | −14.940 | 1.00 | 50.00 | AAAA | N |
| ATOM | 1222 | OD1 | ASN | A | 132 | 77.729 | 234.672 | −12.954 | 1.00 | 50.00 | AAAA | O |
| ATOM | 1223 | H | ASN | A | 132 | 78.988 | 232.373 | −13.975 | 0.00 | 0.00 | AAAA | H |
| ATOM | 1224 | 1HD2 | ASN | A | 132 | 77.069 | 235.979 | −15.028 | 0.00 | 0.00 | AAAA | H |
| ATOM | 1225 | 2HD2 | ASN | A | 132 | 78.665 | 236.010 | −15.666 | 0.00 | 0.00 | AAAA | H |
| ATOM | 1226 | N | ASP | A | 133 | 82.289 | 232.584 | −14.219 | 1.00 | 50.00 | AAAA | N |
| ATOM | 1227 | CA | ASP | A | 133 | 83.691 | 232.165 | −14.227 | 1.00 | 50.00 | AAAA | C |
| ATOM | 1228 | C | ASP | A | 133 | 84.217 | 231.929 | −15.642 | 1.00 | 50.00 | AAAA | C |
| ATOM | 1229 | O | ASP | A | 133 | 83.629 | 231.205 | −16.434 | 1.00 | 50.00 | AAAA | O |
| ATOM | 1230 | CB | ASP | A | 133 | 83.793 | 230.910 | −13.346 | 1.00 | 50.00 | AAAA | C |
| ATOM | 1231 | CG | ASP | A | 133 | 85.215 | 230.497 | −13.017 | 1.00 | 50.00 | AAAA | C |
| ATOM | 1232 | OD1 | ASP | A | 133 | 86.060 | 230.490 | −13.904 | 1.00 | 50.00 | AAAA | O |
| ATOM | 1233 | OD2 | ASP | A | 133 | 85.475 | 230.182 | −11.858 | 1.00 | 50.00 | AAAA | O |
| ATOM | 1234 | H | ASP | A | 133 | 81.624 | 232.064 | −13.685 | 0.00 | 0.00 | AAAA | H |
| ATOM | 1235 | N | GLN | A | 134 | 85.350 | 232.604 | −15.918 | 1.00 | 50.00 | AAAA | N |
| ATOM | 1236 | CA | GLN | A | 134 | 85.954 | 232.440 | −17.240 | 1.00 | 50.00 | AAAA | C |
| ATOM | 1237 | C | GLN | A | 134 | 86.638 | 231.090 | −17.374 | 1.00 | 50.00 | AAAA | C |
| ATOM | 1238 | O | GLN | A | 134 | 87.060 | 230.494 | −16.393 | 1.00 | 50.00 | AAAA | O |
| ATOM | 1239 | CB | GLN | A | 134 | 86.937 | 233.589 | −17.518 | 1.00 | 50.00 | AAAA | C |
| ATOM | 1240 | CG | GLN | A | 134 | 88.115 | 233.619 | −16.536 | 1.00 | 50.00 | AAAA | C |
| ATOM | 1241 | CD | GLN | A | 134 | 88.985 | 234.847 | −16.702 | 1.00 | 50.00 | AAAA | C |
| ATOM | 1242 | NE2 | GLN | A | 134 | 90.295 | 234.563 | −16.778 | 1.00 | 50.00 | AAAA | N |
| ATOM | 1243 | OE1 | GLN | A | 134 | 88.522 | 235.978 | −16.743 | 1.00 | 50.00 | AAAA | O |
| ATOM | 1244 | H | GLN | A | 134 | 85.818 | 233.105 | −15.192 | 0.00 | 0.00 | AAAA | H |
| ATOM | 1245 | 1HE2 | GLN | A | 134 | 90.986 | 235.271 | −16.924 | 0.00 | 0.00 | AAAA | H |
| ATOM | 1246 | 2HE2 | GLN | A | 134 | 90.606 | 233.618 | −16.699 | 0.00 | 0.00 | AAAA | H |
| ATOM | 1247 | N | LYS | A | 135 | 86.764 | 230.644 | −18.636 | 1.00 | 50.00 | AAAA | N |
| ATOM | 1248 | CA | LYS | A | 135 | 87.459 | 229.377 | −18.877 | 1.00 | 50.00 | AAAA | C |
| ATOM | 1249 | C | LYS | A | 135 | 88.861 | 229.326 | −18.307 | 1.00 | 50.00 | AAAA | C |
| ATOM | 1250 | O | LYS | A | 135 | 89.347 | 228.298 | −17.870 | 1.00 | 50.00 | AAAA | O |
| ATOM | 1251 | CB | LYS | A | 135 | 87.553 | 229.118 | −20.370 | 1.00 | 50.00 | AAAA | C |
| ATOM | 1252 | CG | LYS | A | 135 | 88.283 | 227.821 | −20.716 | 1.00 | 50.00 | AAAA | C |
| ATOM | 1253 | CD | LYS | A | 135 | 88.352 | 227.599 | −22.210 | 1.00 | 50.00 | AAAA | C |
| ATOM | 1254 | CE | LYS | A | 135 | 89.073 | 228.676 | −23.015 | 1.00 | 50.00 | AAAA | C |
| ATOM | 1255 | NZ | LYS | A | 135 | 88.937 | 228.308 | −24.431 | 1.00 | 50.00 | AAAA | N |
| ATOM | 1256 | H | LYS | A | 135 | 86.360 | 231.147 | −19.398 | 0.00 | 0.00 | AAAA | H |
| ATOM | 1257 | 1HZ | LYS | A | 135 | 89.436 | 228.996 | −25.029 | 0.00 | 0.00 | AAAA | H |
| ATOM | 1258 | 2HZ | LYS | A | 135 | 87.928 | 228.302 | −24.683 | 0.00 | 0.00 | AAAA | H |
| ATOM | 1259 | 3HZ | LYS | A | 135 | 89.327 | 227.360 | −24.593 | 0.00 | 0.00 | AAAA | H |
| ATOM | 1260 | N | LYS | A | 136 | 89.485 | 230.512 | −18.350 | 1.00 | 50.00 | AAAA | N |
| ATOM | 1261 | CA | LYS | A | 136 | 90.858 | 230.608 | −17.882 | 1.00 | 50.00 | AAAA | C |
| ATOM | 1262 | C | LYS | A | 136 | 91.004 | 230.407 | −16.376 | 1.00 | 50.00 | AAAA | C |
| ATOM | 1263 | O | LYS | A | 136 | 92.006 | 229.886 | −15.916 | 1.00 | 50.00 | AAAA | O |
| ATOM | 1264 | CB | LYS | A | 136 | 91.413 | 231.946 | −18.371 | 1.00 | 50.00 | AAAA | C |
| ATOM | 1265 | CG | LYS | A | 136 | 92.840 | 232.299 | −17.953 | 1.00 | 50.00 | AAAA | C |
| ATOM | 1266 | CD | LYS | A | 136 | 93.898 | 231.451 | −18.653 | 1.00 | 50.00 | AAAA | C |
| ATOM | 1267 | CE | LYS | A | 136 | 95.307 | 231.787 | −18.165 | 1.00 | 50.00 | AAAA | C |
| ATOM | 1268 | NZ | LYS | A | 136 | 95.446 | 231.378 | −16.759 | 1.00 | 50.00 | AAAA | N |
| ATOM | 1269 | H | LYS | A | 136 | 89.021 | 231.319 | −18.712 | 0.00 | 0.00 | AAAA | H |
| ATOM | 1270 | 1HZ | LYS | A | 136 | 96.417 | 231.568 | −16.439 | 0.00 | 0.00 | AAAA | H |
| ATOM | 1271 | 2HZ | LYS | A | 136 | 95.252 | 230.360 | −16.684 | 0.00 | 0.00 | AAAA | H |
| ATOM | 1272 | 3HZ | LYS | A | 136 | 94.776 | 231.902 | −16.160 | 0.00 | 0.00 | AAAA | H |
| ATOM | 1273 | N | LEU | A | 137 | 89.957 | 230.811 | −15.622 | 1.00 | 50.00 | AAAA | N |
| ATOM | 1274 | CA | LEU | A | 137 | 89.976 | 230.419 | −14.208 | 1.00 | 50.00 | AAAA | C |
| ATOM | 1275 | C | LEU | A | 137 | 89.693 | 228.940 | −14.006 | 1.00 | 50.00 | AAAA | C |
| ATOM | 1276 | O | LEU | A | 137 | 90.314 | 228.283 | −13.189 | 1.00 | 50.00 | AAAA | O |
| ATOM | 1277 | CB | LEU | A | 137 | 88.995 | 231.218 | −13.348 | 1.00 | 50.00 | AAAA | C |
| ATOM | 1278 | CG | LEU | A | 137 | 89.134 | 232.737 | −13.310 | 1.00 | 50.00 | AAAA | C |
| ATOM | 1279 | CD1 | LEU | A | 137 | 87.891 | 233.385 | −12.692 | 1.00 | 50.00 | AAAA | C |
| ATOM | 1280 | CD2 | LEU | A | 137 | 90.428 | 233.202 | −12.644 | 1.00 | 50.00 | AAAA | C |
| ATOM | 1281 | H | LEU | A | 137 | 89.135 | 231.187 | −16.046 | 0.00 | 0.00 | AAAA | H |
| ATOM | 1282 | N | MET | A | 138 | 88.742 | 228.415 | −14.801 | 1.00 | 50.00 | AAAA | N |
| ATOM | 1283 | CA | MET | A | 138 | 88.449 | 226.982 | −14.658 | 1.00 | 50.00 | AAAA | C |
| ATOM | 1284 | C | MET | A | 138 | 89.622 | 226.054 | −14.962 | 1.00 | 50.00 | AAAA | C |
| ATOM | 1285 | O | MET | A | 138 | 89.851 | 225.054 | −14.295 | 1.00 | 50.00 | AAAA | O |
| ATOM | 1286 | CB | MET | A | 138 | 87.252 | 226.596 | −15.518 | 1.00 | 50.00 | AAAA | C |
| ATOM | 1287 | CG | MET | A | 138 | 85.964 | 227.352 | −15.194 | 1.00 | 50.00 | AAAA | C |
| ATOM | 1288 | SD | MET | A | 138 | 84.625 | 226.899 | −16.306 | 1.00 | 50.00 | AAAA | S |
| ATOM | 1289 | CE | MET | A | 138 | 83.358 | 227.966 | −15.613 | 1.00 | 50.00 | AAAA | C |
| ATOM | 1290 | H | MET | A | 138 | 88.239 | 228.995 | −15.442 | 0.00 | 0.00 | AAAA | H |
| ATOM | 1291 | N | SER | A | 139 | 90.362 | 226.468 | −16.005 | 1.00 | 50.00 | AAAA | N |
| ATOM | 1292 | CA | SER | A | 139 | 91.539 | 225.723 | −16.439 | 1.00 | 50.00 | AAAA | C |
| ATOM | 1293 | C | SER | A | 139 | 92.765 | 225.912 | −15.564 | 1.00 | 50.00 | AAAA | C |
| ATOM | 1294 | O | SER | A | 139 | 93.564 | 224.999 | −15.392 | 1.00 | 50.00 | AAAA | O |
| ATOM | 1295 | CB | SER | A | 139 | 91.889 | 226.081 | −17.886 | 1.00 | 50.00 | AAAA | C |
| ATOM | 1296 | OG | SER | A | 139 | 90.763 | 225.860 | −18.745 | 1.00 | 50.00 | AAAA | O |
| ATOM | 1297 | H | SER | A | 139 | 90.119 | 227.319 | −16.461 | 0.00 | 0.00 | AAAA | H |
| ATOM | 1298 | HG | SER | A | 139 | 91.030 | 226.163 | −19.603 | 0.00 | 0.00 | AAAA | H |
| ATOM | 1299 | N | ASN | A | 140 | 92.882 | 227.148 | −15.039 | 1.00 | 50.00 | AAAA | N |
| ATOM | 1300 | CA | ASN | A | 140 | 94.050 | 227.452 | −14.212 | 1.00 | 50.00 | AAAA | C |
| ATOM | 1301 | C | ASN | A | 140 | 93.876 | 227.153 | −12.727 | 1.00 | 50.00 | AAAA | C |
| ATOM | 1302 | O | ASN | A | 140 | 94.833 | 227.098 | −11.967 | 1.00 | 50.00 | AAAA | O |
| ATOM | 1303 | CB | ASN | A | 140 | 94.475 | 228.906 | −14.455 | 1.00 | 50.00 | AAAA | C |
| ATOM | 1304 | CG | ASN | A | 140 | 95.806 | 229.233 | −13.814 | 1.00 | 50.00 | AAAA | C |
| ATOM | 1305 | ND2 | ASN | A | 140 | 95.733 | 230.240 | −12.929 | 1.00 | 50.00 | AAAA | N |
| ATOM | 1306 | OD1 | ASN | A | 140 | 96.828 | 228.622 | −14.096 | 1.00 | 50.00 | AAAA | O |
| ATOM | 1307 | H | ASN | A | 140 | 92.182 | 227.840 | −15.217 | 0.00 | 0.00 | AAAA | H |
| ATOM | 1308 | 1HD2 | ASN | A | 140 | 96.554 | 230.507 | −12.427 | 0.00 | 0.00 | AAAA | H |
| ATOM | 1309 | 2HD2 | ASN | A | 140 | 94.871 | 230.718 | −12.761 | 0.00 | 0.00 | AAAA | H |
| ATOM | 1310 | N | ASN | A | 141 | 92.608 | 226.959 | −12.341 | 1.00 | 50.00 | AAAA | N |
| ATOM | 1311 | CA | ASN | A | 141 | 92.387 | 226.651 | −10.931 | 1.00 | 50.00 | AAAA | C |
| ATOM | 1312 | C | ASN | A | 141 | 92.172 | 225.163 | −10.721 | 1.00 | 50.00 | AAAA | C |
| ATOM | 1313 | O | ASN | A | 141 | 92.210 | 224.378 | −11.656 | 1.00 | 50.00 | AAAA | O |
| ATOM | 1314 | CB | ASN | A | 141 | 91.228 | 227.474 | −10.345 | 1.00 | 50.00 | AAAA | C |
| ATOM | 1315 | CG | ASN | A | 141 | 91.490 | 228.967 | −10.476 | 1.00 | 50.00 | AAAA | C |
| ATOM | 1316 | ND2 | ASN | A | 141 | 90.368 | 229.705 | −10.525 | 1.00 | 50.00 | AAAA | N |
| ATOM | 1317 | OD1 | ASN | A | 141 | 92.620 | 229.436 | −10.524 | 1.00 | 50.00 | AAAA | O |
| ATOM | 1318 | H | ASN | A | 141 | 91.861 | 226.934 | −13.003 | 0.00 | 0.00 | AAAA | H |
| ATOM | 1319 | 1HD2 | ASN | A | 141 | 90.429 | 230.700 | −10.594 | 0.00 | 0.00 | AAAA | H |
| ATOM | 1320 | 2HD2 | ASN | A | 141 | 89.465 | 229.276 | −10.508 | 0.00 | 0.00 | AAAA | H |
| ATOM | 1321 | N | VAL | A | 142 | 91.939 | 224.791 | −9.450 | 1.00 | 50.00 | AAAA | N |
| ATOM | 1322 | CA | VAL | A | 142 | 91.685 | 223.371 | −9.189 | 1.00 | 50.00 | AAAA | C |
| ATOM | 1323 | C | VAL | A | 142 | 90.199 | 223.060 | −9.001 | 1.00 | 50.00 | AAAA | C |
| ATOM | 1324 | O | VAL | A | 142 | 89.787 | 222.037 | −8.478 | 1.00 | 50.00 | AAAA | O |
| ATOM | 1325 | CB | VAL | A | 142 | 92.579 | 222.899 | −8.018 | 1.00 | 50.00 | AAAA | C |
| ATOM | 1326 | CG1 | VAL | A | 142 | 92.177 | 223.529 | −6.683 | 1.00 | 50.00 | AAAA | C |
| ATOM | 1327 | CG2 | VAL | A | 142 | 92.729 | 221.376 | −7.950 | 1.00 | 50.00 | AAAA | C |
| ATOM | 1328 | H | VAL | A | 142 | 91.927 | 225.463 | −8.712 | 0.00 | 0.00 | AAAA | H |
| ATOM | 1329 | N | GLN | A | 143 | 89.390 | 224.039 | −9.461 | 1.00 | 50.00 | AAAA | N |
| ATOM | 1330 | CA | GLN | A | 143 | 87.943 | 223.923 | −9.271 | 1.00 | 50.00 | AAAA | C |
| ATOM | 1331 | C | GLN | A | 143 | 87.312 | 222.683 | −9.885 | 1.00 | 50.00 | AAAA | C |
| ATOM | 1332 | O | GLN | A | 143 | 86.517 | 221.988 | −9.268 | 1.00 | 50.00 | AAAA | O |
| ATOM | 1333 | CB | GLN | A | 143 | 87.248 | 225.148 | −9.850 | 1.00 | 50.00 | AAAA | C |
| ATOM | 1334 | CG | GLN | A | 143 | 87.615 | 226.483 | −9.219 | 1.00 | 50.00 | AAAA | C |
| ATOM | 1335 | CD | GLN | A | 143 | 86.835 | 227.614 | −9.859 | 1.00 | 50.00 | AAAA | C |
| ATOM | 1336 | NE2 | GLN | A | 143 | 86.507 | 228.604 | −9.016 | 1.00 | 50.00 | AAAA | N |
| ATOM | 1337 | OE1 | GLN | A | 143 | 86.546 | 227.609 | −11.045 | 1.00 | 50.00 | AAAA | O |
| ATOM | 1338 | H | GLN | A | 143 | 89.760 | 224.837 | −9.934 | 0.00 | 0.00 | AAAA | H |
| ATOM | 1339 | 1HE2 | GLN | A | 143 | 86.014 | 229.405 | −9.353 | 0.00 | 0.00 | AAAA | H |
| ATOM | 1340 | 2HE2 | GLN | A | 143 | 86.746 | 228.561 | −8.052 | 0.00 | 0.00 | AAAA | H |
| ATOM | 1341 | N | ILE | A | 144 | 87.703 | 222.449 | −11.153 | 1.00 | 50.00 | AAAA | N |
| ATOM | 1342 | CA | ILE | A | 144 | 87.138 | 221.296 | −11.854 | 1.00 | 50.00 | AAAA | C |
| ATOM | 1343 | C | ILE | A | 144 | 87.576 | 219.946 | −11.304 | 1.00 | 50.00 | AAAA | C |
| ATOM | 1344 | O | ILE | A | 144 | 88.708 | 219.722 | −10.904 | 1.00 | 50.00 | AAAA | O |
| ATOM | 1345 | CB | ILE | A | 144 | 87.403 | 221.384 | −13.364 | 1.00 | 50.00 | AAAA | C |
| ATOM | 1346 | CG1 | ILE | A | 144 | 88.899 | 221.390 | −13.693 | 1.00 | 50.00 | AAAA | C |
| ATOM | 1347 | CG2 | ILE | A | 144 | 86.709 | 222.626 | −13.936 | 1.00 | 50.00 | AAAA | C |
| ATOM | 1348 | CD1 | ILE | A | 144 | 89.198 | 221.292 | −15.190 | 1.00 | 50.00 | AAAA | C |
| ATOM | 1349 | H | ILE | A | 144 | 88.374 | 223.044 | −11.595 | 0.00 | 0.00 | AAAA | H |
| ATOM | 1350 | N | VAL | A | 145 | 86.592 | 219.043 | −11.312 | 1.00 | 50.00 | AAAA | N |
| ATOM | 1351 | CA | VAL | A | 145 | 86.855 | 217.667 | −10.899 | 1.00 | 50.00 | AAAA | C |
| ATOM | 1352 | C | VAL | A | 145 | 85.854 | 216.802 | −11.629 | 1.00 | 50.00 | AAAA | C |
| ATOM | 1353 | O | VAL | A | 145 | 84.783 | 217.264 | −11.985 | 1.00 | 50.00 | AAAA | O |
| ATOM | 1354 | CB | VAL | A | 145 | 86.750 | 217.535 | −9.362 | 1.00 | 50.00 | AAAA | C |
| ATOM | 1355 | CG1 | VAL | A | 145 | 85.365 | 217.931 | −8.835 | 1.00 | 50.00 | AAAA | C |
| ATOM | 1356 | CG2 | VAL | A | 145 | 87.217 | 216.169 | −8.843 | 1.00 | 50.00 | AAAA | C |
| ATOM | 1357 | H | VAL | A | 145 | 85.673 | 219.275 | −11.633 | 0.00 | 0.00 | AAAA | H |
| ATOM | 1358 | N | ARG | A | 146 | 86.230 | 215.543 | −11.865 | 1.00 | 99.99 | AAAA | N |
| ATOM | 1359 | CA | ARG | A | 146 | 85.221 | 214.697 | −12.471 | 1.00 | 99.99 | AAAA | C |
| ATOM | 1360 | C | ARG | A | 146 | 85.361 | 213.322 | −11.898 | 1.00 | 99.99 | AAAA | C |
| ATOM | 1361 | O | ARG | A | 146 | 86.326 | 213.030 | −11.215 | 1.00 | 99.99 | AAAA | O |
| ATOM | 1362 | CB | ARG | A | 146 | 85.344 | 214.701 | −13.997 | 1.00 | 99.99 | AAAA | C |
| ATOM | 1363 | CG | ARG | A | 146 | 86.633 | 214.095 | −14.520 | 1.00 | 99.99 | AAAA | C |
| ATOM | 1364 | CD | ARG | A | 146 | 86.765 | 214.160 | −16.032 | 1.00 | 99.99 | AAAA | C |
| ATOM | 1365 | NE | ARG | A | 146 | 86.850 | 215.553 | −16.451 | 1.00 | 99.99 | AAAA | N |
| ATOM | 1366 | CZ | ARG | A | 146 | 87.134 | 215.869 | −17.725 | 1.00 | 99.99 | AAAA | C |
| ATOM | 1367 | NH1 | ARG | A | 146 | 87.371 | 214.926 | −18.629 | 1.00 | 99.99 | AAAA | N |
| ATOM | 1368 | NH2 | ARG | A | 146 | 87.192 | 217.147 | −18.074 | 1.00 | 99.99 | AAAA | N |
| ATOM | 1369 | H | ARG | A | 146 | 87.101 | 215.166 | −11.547 | 0.00 | 0.00 | AAAA | H |
| ATOM | 1370 | HE | ARG | A | 146 | 86.739 | 216.275 | −15.768 | 0.00 | 0.00 | AAAA | H |
| ATOM | 1371 | 1HH1 | ARG | A | 146 | 87.573 | 215.185 | −19.573 | 0.00 | 0.00 | AAAA | H |
| ATOM | 1372 | 2HH1 | ARG | A | 146 | 87.361 | 213.959 | −18.374 | 0.00 | 0.00 | AAAA | H |
| ATOM | 1373 | 1HH2 | ARG | A | 146 | 87.382 | 217.399 | −19.023 | 0.00 | 0.00 | AAAA | H |
| ATOM | 1374 | 2HH2 | ARG | A | 146 | 87.031 | 217.856 | −17.388 | 0.00 | 0.00 | AAAA | H |
| ATOM | 1375 | N | GLN | A | 147 | 84.385 | 212.475 | −12.193 | 1.00 | 99.99 | AAAA | N |
| ATOM | 1376 | CA | GLN | A | 147 | 84.628 | 211.147 | −11.664 | 1.00 | 99.99 | AAAA | C |
| ATOM | 1377 | C | GLN | A | 147 | 85.586 | 210.316 | −12.520 | 1.00 | 99.99 | AAAA | C |
| ATOM | 1378 | O | GLN | A | 147 | 85.806 | 210.626 | −13.678 | 1.00 | 99.99 | AAAA | O |
| ATOM | 1379 | CB | GLN | A | 147 | 83.297 | 210.477 | −11.521 | 1.00 | 99.99 | AAAA | C |
| ATOM | 1380 | CG | GLN | A | 147 | 82.168 | 211.201 | −10.793 | 1.00 | 99.99 | AAAA | C |
| ATOM | 1381 | CD | GLN | A | 147 | 82.254 | 210.893 | −9.326 | 1.00 | 99.99 | AAAA | C |
| ATOM | 1382 | NE2 | GLN | A | 147 | 81.772 | 209.680 | −9.006 | 1.00 | 99.99 | AAAA | N |
| ATOM | 1383 | OE1 | GLN | A | 147 | 82.748 | 211.687 | −8.539 | 1.00 | 99.99 | AAAA | O |
| ATOM | 1384 | H | GLN | A | 147 | 83.589 | 212.728 | −12.742 | 0.00 | 0.00 | AAAA | H |
| ATOM | 1385 | 1HE2 | GLN | A | 147 | 81.858 | 209.381 | −8.057 | 0.00 | 0.00 | AAAA | H |
| ATOM | 1386 | 2HE2 | GLN | A | 147 | 81.355 | 209.061 | −9.672 | 0.00 | 0.00 | AAAA | H |
| ATOM | 1387 | N | GLN | A | 148 | 86.143 | 209.258 | −11.895 | 1.00 | 99.99 | AAAA | N |
| ATOM | 1388 | CA | GLN | A | 148 | 87.049 | 208.346 | −12.618 | 1.00 | 99.99 | AAAA | C |
| ATOM | 1389 | C | GLN | A | 148 | 86.477 | 206.948 | −12.714 | 1.00 | 99.99 | AAAA | C |
| ATOM | 1390 | O | GLN | A | 148 | 87.016 | 206.047 | −13.348 | 1.00 | 99.99 | AAAA | O |
| ATOM | 1391 | CB | GLN | A | 148 | 88.416 | 208.287 | −11.920 | 1.00 | 99.99 | AAAA | C |
| ATOM | 1392 | CG | GLN | A | 148 | 89.563 | 207.472 | −12.517 | 1.00 | 99.99 | AAAA | C |
| ATOM | 1393 | CD | GLN | A | 148 | 89.995 | 208.103 | −13.814 | 1.00 | 99.99 | AAAA | C |
| ATOM | 1394 | NE2 | GLN | A | 148 | 90.878 | 209.094 | −13.636 | 1.00 | 99.99 | AAAA | N |
| ATOM | 1395 | OE1 | GLN | A | 148 | 89.566 | 207.741 | −14.900 | 1.00 | 99.99 | AAAA | O |
| ATOM | 1396 | H | GLN | A | 148 | 85.962 | 209.075 | −10.931 | 0.00 | 0.00 | AAAA | H |
| ATOM | 1397 | 1HE2 | GLN | A | 148 | 91.211 | 209.582 | −14.442 | 0.00 | 0.00 | AAAA | H |
| ATOM | 1398 | 2HE2 | GLN | A | 148 | 91.204 | 209.370 | −12.732 | 0.00 | 0.00 | AAAA | H |
| ATOM | 1399 | N | SER | A | 149 | 85.314 | 206.794 | −12.053 | 1.00 | 99.99 | AAAA | N |
| ATOM | 1400 | CA | SER | A | 149 | 84.618 | 205.540 | −12.259 | 1.00 | 99.99 | AAAA | C |
| ATOM | 1401 | C | SER | A | 149 | 84.181 | 205.445 | −13.702 | 1.00 | 99.99 | AAAA | C |
| ATOM | 1402 | O | SER | A | 149 | 83.968 | 206.405 | −14.431 | 1.00 | 99.99 | AAAA | O |
| ATOM | 1403 | CB | SER | A | 149 | 83.475 | 205.394 | −11.248 | 1.00 | 99.99 | AAAA | C |
| ATOM | 1404 | OG | SER | A | 149 | 82.646 | 204.265 | −11.548 | 1.00 | 99.99 | AAAA | O |
| ATOM | 1405 | H | SER | A | 149 | 84.849 | 207.542 | −11.590 | 0.00 | 0.00 | AAAA | H |
| ATOM | 1406 | HG | SER | A | 149 | 81.896 | 204.327 | −10.971 | 0.00 | 0.00 | AAAA | H |
| ATOM | 1407 | N | TYR | A | 150 | 84.161 | 204.190 | −14.075 | 1.00 | 99.99 | AAAA | N |
| ATOM | 1408 | CA | TYR | A | 150 | 84.050 | 203.862 | −15.473 | 1.00 | 99.99 | AAAA | C |
| ATOM | 1409 | C | TYR | A | 150 | 82.803 | 202.998 | −15.717 | 1.00 | 99.99 | AAAA | C |
| ATOM | 1410 | O | TYR | A | 150 | 82.588 | 202.427 | −16.765 | 1.00 | 99.99 | AAAA | O |
| ATOM | 1411 | CB | TYR | A | 150 | 85.422 | 203.265 | −15.798 | 1.00 | 99.99 | AAAA | C |
| ATOM | 1412 | CG | TYR | A | 150 | 85.673 | 202.118 | −14.855 | 1.00 | 99.99 | AAAA | C |
| ATOM | 1413 | CD1 | TYR | A | 150 | 86.375 | 202.242 | −13.626 | 1.00 | 99.99 | AAAA | C |
| ATOM | 1414 | CD2 | TYR | A | 150 | 85.130 | 200.917 | −15.290 | 1.00 | 99.99 | AAAA | C |
| ATOM | 1415 | CE1 | TYR | A | 150 | 86.557 | 201.094 | −12.828 | 1.00 | 99.99 | AAAA | C |
| ATOM | 1416 | CE2 | TYR | A | 150 | 85.349 | 199.796 | −14.509 | 1.00 | 99.99 | AAAA | C |
| ATOM | 1417 | CZ | TYR | A | 150 | 86.079 | 199.875 | −13.334 | 1.00 | 99.99 | AAAA | C |
| ATOM | 1418 | OH | TYR | A | 150 | 86.329 | 198.682 | −12.729 | 1.00 | 99.99 | AAAA | O |
| ATOM | 1419 | H | TYR | A | 150 | 84.404 | 203.515 | −13.381 | 0.00 | 0.00 | AAAA | H |
| ATOM | 1420 | HH | TYR | A | 150 | 85.968 | 197.975 | −13.253 | 0.00 | 0.00 | AAAA | H |
| ATOM | 1421 | N | SER | A | 151 | 81.949 | 202.944 | −14.680 | 1.00 | 99.99 | AAAA | N |
| ATOM | 1422 | CA | SER | A | 151 | 80.574 | 202.420 | −14.804 | 1.00 | 99.99 | AAAA | C |
| ATOM | 1423 | C | SER | A | 151 | 79.660 | 203.542 | −14.892 | 1.00 | 99.99 | AAAA | C |
| ATOM | 1424 | O | SER | A | 151 | 78.593 | 203.494 | −15.481 | 1.00 | 99.99 | AAAA | O |
| ATOM | 1425 | CB | SER | A | 151 | 79.942 | 201.809 | −13.568 | 1.00 | 99.99 | AAAA | C |
| ATOM | 1426 | OG | SER | A | 151 | 79.546 | 200.478 | −13.890 | 1.00 | 99.99 | AAAA | O |
| ATOM | 1427 | H | SER | A | 151 | 82.264 | 203.331 | −13.817 | 0.00 | 0.00 | AAAA | H |
| ATOM | 1428 | HG | SER | A | 151 | 79.366 | 200.044 | −13.068 | 0.00 | 0.00 | AAAA | H |
| ATOM | 1429 | N | ILE | A | 152 | 80.174 | 204.601 | −14.268 | 1.00 | 99.99 | AAAA | N |
| ATOM | 1430 | CA | ILE | A | 152 | 79.464 | 205.798 | −14.605 | 1.00 | 99.99 | AAAA | C |
| ATOM | 1431 | C | ILE | A | 152 | 79.682 | 206.176 | −16.041 | 1.00 | 99.99 | AAAA | C |
| ATOM | 1432 | O | ILE | A | 152 | 78.940 | 206.981 | −16.519 | 1.00 | 99.99 | AAAA | O |
| ATOM | 1433 | CB | ILE | A | 152 | 79.816 | 206.916 | −13.665 | 1.00 | 99.99 | AAAA | C |
| ATOM | 1434 | CG1 | ILE | A | 152 | 81.165 | 207.474 | −14.033 | 1.00 | 99.99 | AAAA | C |
| ATOM | 1435 | CG2 | ILE | A | 152 | 79.832 | 206.326 | −12.271 | 1.00 | 99.99 | AAAA | C |
| ATOM | 1436 | CD1 | ILE | A | 152 | 81.733 | 208.087 | −12.800 | 1.00 | 99.99 | AAAA | C |
| ATOM | 1437 | H | ILE | A | 152 | 81.066 | 204.606 | −13.809 | 0.00 | 0.00 | AAAA | H |
| ATOM | 1438 | N | MET | A | 153 | 80.650 | 205.523 | −16.713 | 1.00 | 50.00 | AAAA | N |
| ATOM | 1439 | CA | MET | A | 153 | 80.333 | 204.798 | −17.947 | 1.00 | 50.00 | AAAA | C |
| ATOM | 1440 | C | MET | A | 153 | 81.602 | 204.391 | −18.640 | 1.00 | 50.00 | AAAA | C |
| ATOM | 1441 | O | MET | A | 153 | 82.586 | 205.116 | −18.648 | 1.00 | 50.00 | AAAA | O |
| ATOM | 1442 | CB | MET | A | 153 | 79.414 | 205.519 | −18.942 | 1.00 | 50.00 | AAAA | C |
| ATOM | 1443 | CG | MET | A | 153 | 78.594 | 204.558 | −19.796 | 1.00 | 50.00 | AAAA | C |
| ATOM | 1444 | SD | MET | A | 153 | 77.556 | 203.500 | −18.776 | 1.00 | 50.00 | AAAA | S |
| ATOM | 1445 | CE | MET | A | 153 | 77.320 | 202.167 | −19.957 | 1.00 | 50.00 | AAAA | C |
| ATOM | 1446 | H | MET | A | 153 | 81.486 | 205.278 | −16.226 | 0.00 | 0.00 | AAAA | H |
| ATOM | 1447 | N | SER | A | 154 | 81.530 | 203.178 | −19.193 | 1.00 | 50.00 | AAAA | N |
| ATOM | 1448 | CA | SER | A | 154 | 82.596 | 202.677 | −20.041 | 1.00 | 50.00 | AAAA | C |
| ATOM | 1449 | C | SER | A | 154 | 82.022 | 201.559 | −20.820 | 1.00 | 50.00 | AAAA | C |
| ATOM | 1450 | O | SER | A | 154 | 81.300 | 200.703 | −20.324 | 1.00 | 50.00 | AAAA | O |
| ATOM | 1451 | CB | SER | A | 154 | 83.793 | 202.108 | −19.278 | 1.00 | 50.00 | AAAA | C |
| ATOM | 1452 | OG | SER | A | 154 | 84.731 | 201.443 | −20.130 | 1.00 | 50.00 | AAAA | O |
| ATOM | 1453 | H | SER | A | 154 | 80.711 | 202.618 | −19.054 | 0.00 | 0.00 | AAAA | H |
| ATOM | 1454 | HG | SER | A | 154 | 85.551 | 201.430 | −19.652 | 0.00 | 0.00 | AAAA | H |
| ATOM | 1455 | N | ILE | A | 155 | 82.446 | 201.619 | −22.071 | 1.00 | 50.00 | AAAA | N |
| ATOM | 1456 | CA | ILE | A | 155 | 82.322 | 200.439 | −22.882 | 1.00 | 50.00 | AAAA | C |
| ATOM | 1457 | C | ILE | A | 155 | 83.646 | 200.181 | −23.503 | 1.00 | 50.00 | AAAA | C |
| ATOM | 1458 | O | ILE | A | 155 | 84.257 | 200.958 | −24.218 | 1.00 | 50.00 | AAAA | O |
| ATOM | 1459 | CB | ILE | A | 155 | 81.253 | 200.565 | −23.938 | 1.00 | 50.00 | AAAA | C |
| ATOM | 1460 | CG1 | ILE | A | 155 | 81.462 | 201.904 | −24.614 | 1.00 | 50.00 | AAAA | C |
| ATOM | 1461 | CG2 | ILE | A | 155 | 79.904 | 200.439 | −23.247 | 1.00 | 50.00 | AAAA | C |
| ATOM | 1462 | CD1 | ILE | A | 155 | 80.587 | 202.092 | −25.817 | 1.00 | 50.00 | AAAA | C |
| ATOM | 1463 | H | ILE | A | 155 | 82.953 | 202.414 | −22.402 | 0.00 | 0.00 | AAAA | H |
| ATOM | 1464 | N | ILE | A | 156 | 84.050 | 198.990 | −23.133 | 1.00 | 50.00 | AAAA | N |
| ATOM | 1465 | CA | ILE | A | 156 | 85.315 | 198.475 | −23.606 | 1.00 | 50.00 | AAAA | C |
| ATOM | 1466 | C | ILE | A | 156 | 85.382 | 198.149 | −25.076 | 1.00 | 50.00 | AAAA | C |
| ATOM | 1467 | O | ILE | A | 156 | 86.415 | 198.287 | −25.714 | 1.00 | 50.00 | AAAA | O |
| ATOM | 1468 | CB | ILE | A | 156 | 85.635 | 197.280 | −22.753 | 1.00 | 50.00 | AAAA | C |
| ATOM | 1469 | CG1 | ILE | A | 156 | 84.410 | 196.348 | −22.568 | 1.00 | 50.00 | AAAA | C |
| ATOM | 1470 | CG2 | ILE | A | 156 | 86.203 | 197.929 | −21.500 | 1.00 | 50.00 | AAAA | C |
| ATOM | 1471 | CD1 | ILE | A | 156 | 84.654 | 194.928 | −22.061 | 1.00 | 50.00 | AAAA | C |
| ATOM | 1472 | H | ILE | A | 156 | 83.517 | 198.533 | −22.422 | 0.00 | 0.00 | AAAA | H |
| ATOM | 1473 | N | LYS | A | 157 | 84.210 | 197.737 | −25.579 | 1.00 | 50.00 | AAAA | N |
| ATOM | 1474 | CA | LYS | A | 157 | 84.089 | 197.400 | −26.990 | 1.00 | 50.00 | AAAA | C |
| ATOM | 1475 | C | LYS | A | 157 | 84.291 | 198.584 | −27.923 | 1.00 | 50.00 | AAAA | C |
| ATOM | 1476 | O | LYS | A | 157 | 84.973 | 198.501 | −28.936 | 1.00 | 50.00 | AAAA | O |
| ATOM | 1477 | CB | LYS | A | 157 | 82.729 | 196.729 | −27.180 | 1.00 | 50.00 | AAAA | C |
| ATOM | 1478 | CG | LYS | A | 157 | 82.493 | 196.152 | −28.571 | 1.00 | 50.00 | AAAA | C |
| ATOM | 1479 | CD | LYS | A | 157 | 83.458 | 195.011 | −28.884 | 1.00 | 50.00 | AAAA | C |
| ATOM | 1480 | CE | LYS | A | 157 | 83.266 | 194.469 | −30.299 | 1.00 | 50.00 | AAAA | C |
| ATOM | 1481 | NZ | LYS | A | 157 | 83.611 | 195.514 | −31.274 | 1.00 | 50.00 | AAAA | N |
| ATOM | 1482 | H | LYS | A | 157 | 83.424 | 197.647 | −24.969 | 0.00 | 0.00 | AAAA | H |
| ATOM | 1483 | 1HZ | LYS | A | 157 | 83.492 | 195.138 | −32.238 | 0.00 | 0.00 | AAAA | H |
| ATOM | 1484 | 2HZ | LYS | A | 157 | 84.604 | 195.791 | −31.139 | 0.00 | 0.00 | AAAA | H |
| ATOM | 1485 | 3HZ | LYS | A | 157 | 83.001 | 196.348 | −31.148 | 0.00 | 0.00 | AAAA | H |
| ATOM | 1486 | N | GLU | A | 158 | 83.671 | 199.703 | −27.511 | 1.00 | 50.00 | AAAA | N |
| ATOM | 1487 | CA | GLU | A | 158 | 83.781 | 200.885 | −28.359 | 1.00 | 50.00 | AAAA | C |
| ATOM | 1488 | C | GLU | A | 158 | 84.729 | 201.938 | −27.819 | 1.00 | 50.00 | AAAA | C |
| ATOM | 1489 | O | GLU | A | 158 | 84.889 | 203.012 | −28.387 | 1.00 | 50.00 | AAAA | O |
| ATOM | 1490 | CB | GLU | A | 158 | 82.412 | 201.506 | −28.630 | 1.00 | 50.00 | AAAA | C |
| ATOM | 1491 | CG | GLU | A | 158 | 81.379 | 200.632 | −29.358 | 1.00 | 50.00 | AAAA | C |
| ATOM | 1492 | CD | GLU | A | 158 | 80.751 | 199.519 | −28.518 | 1.00 | 50.00 | AAAA | C |
| ATOM | 1493 | OE1 | GLU | A | 158 | 80.937 | 199.442 | −27.304 | 1.00 | 50.00 | AAAA | O |
| ATOM | 1494 | OE2 | GLU | A | 158 | 80.035 | 198.708 | −29.101 | 1.00 | 50.00 | AAAA | O |
| ATOM | 1495 | H | GLU | A | 158 | 83.142 | 199.737 | −26.663 | 0.00 | 0.00 | AAAA | H |
| ATOM | 1496 | N | GLU | A | 159 | 85.366 | 201.553 | −26.690 | 1.00 | 50.00 | AAAA | N |
| ATOM | 1497 | CA | GLU | A | 159 | 86.368 | 202.375 | −26.021 | 1.00 | 50.00 | AAAA | C |
| ATOM | 1498 | C | GLU | A | 159 | 85.900 | 203.779 | −25.662 | 1.00 | 50.00 | AAAA | C |
| ATOM | 1499 | O | GLU | A | 159 | 86.628 | 204.753 | −25.786 | 1.00 | 50.00 | AAAA | O |
| ATOM | 1500 | CB | GLU | A | 159 | 87.693 | 202.320 | −26.806 | 1.00 | 50.00 | AAAA | C |
| ATOM | 1501 | CG | GLU | A | 159 | 88.133 | 200.858 | −27.008 | 1.00 | 50.00 | AAAA | C |
| ATOM | 1502 | CD | GLU | A | 159 | 89.362 | 200.701 | −27.892 | 1.00 | 50.00 | AAAA | C |
| ATOM | 1503 | OE1 | GLU | A | 159 | 89.442 | 201.342 | −28.939 | 1.00 | 50.00 | AAAA | O |
| ATOM | 1504 | OE2 | GLU | A | 159 | 90.230 | 199.899 | −27.540 | 1.00 | 50.00 | AAAA | O |
| ATOM | 1505 | H | GLU | A | 159 | 85.142 | 200.678 | −26.262 | 0.00 | 0.00 | AAAA | H |
| ATOM | 1506 | N | VAL | A | 160 | 84.635 | 203.845 | −25.193 | 1.00 | 50.00 | AAAA | N |
| ATOM | 1507 | CA | VAL | A | 160 | 84.221 | 205.162 | −24.713 | 1.00 | 50.00 | AAAA | C |
| ATOM | 1508 | C | VAL | A | 160 | 84.069 | 205.185 | −23.210 | 1.00 | 50.00 | AAAA | C |
| ATOM | 1509 | O | VAL | A | 160 | 83.412 | 204.357 | −22.595 | 1.00 | 50.00 | AAAA | O |
| ATOM | 1510 | CB | VAL | A | 160 | 83.002 | 205.767 | −25.461 | 1.00 | 50.00 | AAAA | C |
| ATOM | 1511 | CG1 | VAL | A | 160 | 81.622 | 205.316 | −24.997 | 1.00 | 50.00 | AAAA | C |
| ATOM | 1512 | CG2 | VAL | A | 160 | 83.029 | 207.287 | −25.357 | 1.00 | 50.00 | AAAA | C |
| ATOM | 1513 | H | VAL | A | 160 | 84.059 | 203.033 | −25.096 | 0.00 | 0.00 | AAAA | H |
| ATOM | 1514 | N | LEU | A | 161 | 84.763 | 206.161 | −22.629 | 1.00 | 50.00 | AAAA | N |
| ATOM | 1515 | CA | LEU | A | 161 | 84.557 | 206.323 | −21.200 | 1.00 | 50.00 | AAAA | C |
| ATOM | 1516 | C | LEU | A | 161 | 83.777 | 207.596 | −20.959 | 1.00 | 50.00 | AAAA | C |
| ATOM | 1517 | O | LEU | A | 161 | 84.150 | 208.673 | −21.400 | 1.00 | 50.00 | AAAA | O |
| ATOM | 1518 | CB | LEU | A | 161 | 85.890 | 206.373 | −20.457 | 1.00 | 50.00 | AAAA | C |
| ATOM | 1519 | CG | LEU | A | 161 | 86.838 | 205.187 | −20.628 | 1.00 | 50.00 | AAAA | C |
| ATOM | 1520 | CD1 | LEU | A | 161 | 88.286 | 205.561 | −20.352 | 1.00 | 50.00 | AAAA | C |
| ATOM | 1521 | CD2 | LEU | A | 161 | 86.499 | 204.064 | −19.674 | 1.00 | 50.00 | AAAA | C |
| ATOM | 1522 | H | LEU | A | 161 | 85.303 | 206.801 | −23.177 | 0.00 | 0.00 | AAAA | H |
| ATOM | 1523 | N | ALA | A | 162 | 82.658 | 207.438 | −20.253 | 1.00 | 50.00 | AAAA | N |
| ATOM | 1524 | CA | ALA | A | 162 | 81.976 | 208.671 | −19.902 | 1.00 | 50.00 | AAAA | C |
| ATOM | 1525 | C | ALA | A | 162 | 82.167 | 209.102 | −18.458 | 1.00 | 50.00 | AAAA | C |
| ATOM | 1526 | O | ALA | A | 162 | 82.165 | 208.322 | −17.513 | 1.00 | 50.00 | AAAA | O |
| ATOM | 1527 | CB | ALA | A | 162 | 80.507 | 208.584 | −20.281 | 1.00 | 50.00 | AAAA | C |
| ATOM | 1528 | H | ALA | A | 162 | 82.338 | 206.539 | −19.963 | 0.00 | 0.00 | AAAA | H |
| ATOM | 1529 | N | TYR | A | 163 | 82.389 | 210.418 | −18.370 | 1.00 | 50.00 | AAAA | N |
| ATOM | 1530 | CA | TYR | A | 163 | 82.801 | 211.088 | −17.147 | 1.00 | 50.00 | AAAA | C |
| ATOM | 1531 | C | TYR | A | 163 | 81.867 | 212.227 | −16.809 | 1.00 | 50.00 | AAAA | C |
| ATOM | 1532 | O | TYR | A | 163 | 81.318 | 212.895 | −17.667 | 1.00 | 50.00 | AAAA | O |
| ATOM | 1533 | CB | TYR | A | 163 | 84.195 | 211.691 | −17.310 | 1.00 | 50.00 | AAAA | C |
| ATOM | 1534 | CG | TYR | A | 163 | 85.240 | 210.632 | −17.542 | 1.00 | 50.00 | AAAA | C |
| ATOM | 1535 | CD1 | TYR | A | 163 | 85.849 | 210.019 | −16.430 | 1.00 | 50.00 | AAAA | C |
| ATOM | 1536 | CD2 | TYR | A | 163 | 85.590 | 210.297 | −18.862 | 1.00 | 50.00 | AAAA | C |
| ATOM | 1537 | CE1 | TYR | A | 163 | 86.858 | 209.066 | −16.631 | 1.00 | 50.00 | AAAA | C |
| ATOM | 1538 | CE2 | TYR | A | 163 | 86.593 | 209.342 | −19.060 | 1.00 | 50.00 | AAAA | C |
| ATOM | 1539 | CZ | TYR | A | 163 | 87.225 | 208.748 | −17.950 | 1.00 | 50.00 | AAAA | C |
| ATOM | 1540 | OH | TYR | A | 163 | 88.239 | 207.839 | −18.169 | 1.00 | 50.00 | AAAA | O |
| ATOM | 1541 | H | TYR | A | 163 | 82.265 | 210.976 | −19.185 | 0.00 | 0.00 | AAAA | H |
| ATOM | 1542 | HH | TYR | A | 163 | 88.251 | 207.602 | −19.086 | 0.00 | 0.00 | AAAA | H |
| ATOM | 1543 | N | VAL | A | 164 | 81.737 | 212.459 | −15.501 | 1.00 | 50.00 | AAAA | N |
| ATOM | 1544 | CA | VAL | A | 164 | 80.945 | 213.637 | −15.159 | 1.00 | 50.00 | AAAA | C |
| ATOM | 1545 | C | VAL | A | 164 | 81.752 | 214.683 | −14.410 | 1.00 | 50.00 | AAAA | C |
| ATOM | 1546 | O | VAL | A | 164 | 82.263 | 214.447 | −13.323 | 1.00 | 50.00 | AAAA | O |
| ATOM | 1547 | CB | VAL | A | 164 | 79.641 | 213.230 | −14.443 | 1.00 | 50.00 | AAAA | C |
| ATOM | 1548 | CG1 | VAL | A | 164 | 79.887 | 212.283 | −13.271 | 1.00 | 50.00 | AAAA | C |
| ATOM | 1549 | CG2 | VAL | A | 164 | 78.775 | 214.433 | −14.053 | 1.00 | 50.00 | AAAA | C |
| ATOM | 1550 | H | VAL | A | 164 | 82.204 | 211.888 | −14.828 | 0.00 | 0.00 | AAAA | H |
| ATOM | 1551 | N | VAL | A | 165 | 81.846 | 215.847 | −15.084 | 1.00 | 50.00 | AAAA | N |
| ATOM | 1552 | CA | VAL | A | 165 | 82.557 | 217.005 | −14.536 | 1.00 | 50.00 | AAAA | C |
| ATOM | 1553 | C | VAL | A | 165 | 81.666 | 217.903 | −13.688 | 1.00 | 50.00 | AAAA | C |
| ATOM | 1554 | O | VAL | A | 165 | 80.528 | 218.202 | −14.017 | 1.00 | 50.00 | AAAA | O |
| ATOM | 1555 | CB | VAL | A | 165 | 83.275 | 217.770 | −15.677 | 1.00 | 50.00 | AAAA | C |
| ATOM | 1556 | CG1 | VAL | A | 165 | 82.309 | 218.357 | −16.712 | 1.00 | 50.00 | AAAA | C |
| ATOM | 1557 | CG2 | VAL | A | 165 | 84.273 | 218.811 | −15.157 | 1.00 | 50.00 | AAAA | C |
| ATOM | 1558 | H | VAL | A | 165 | 81.333 | 215.955 | −15.933 | 0.00 | 0.00 | AAAA | H |
| ATOM | 1559 | N | GLN | A | 166 | 82.237 | 218.302 | −12.543 | 1.00 | 50.00 | AAAA | N |
| ATOM | 1560 | CA | GLN | A | 166 | 81.519 | 219.140 | −11.595 | 1.00 | 50.00 | AAAA | C |
| ATOM | 1561 | C | GLN | A | 166 | 82.263 | 220.447 | −11.350 | 1.00 | 50.00 | AAAA | C |
| ATOM | 1562 | O | GLN | A | 166 | 83.434 | 220.460 | −10.983 | 1.00 | 50.00 | AAAA | O |
| ATOM | 1563 | CB | GLN | A | 166 | 81.275 | 218.378 | −10.277 | 1.00 | 50.00 | AAAA | C |
| ATOM | 1564 | CG | GLN | A | 166 | 80.741 | 216.944 | −10.454 | 1.00 | 50.00 | AAAA | C |
| ATOM | 1565 | CD | GLN | A | 166 | 80.260 | 216.359 | −9.135 | 1.00 | 50.00 | AAAA | C |
| ATOM | 1566 | NE2 | GLN | A | 166 | 79.042 | 215.786 | −9.202 | 1.00 | 50.00 | AAAA | N |
| ATOM | 1567 | OE1 | GLN | A | 166 | 80.941 | 216.404 | −8.118 | 1.00 | 50.00 | AAAA | O |
| ATOM | 1568 | H | GLN | A | 166 | 83.184 | 218.060 | −12.375 | 0.00 | 0.00 | AAAA | H |
| ATOM | 1569 | 1HE2 | GLN | A | 166 | 78.686 | 215.316 | −8.395 | 0.00 | 0.00 | AAAA | H |
| ATOM | 1570 | 2HE2 | GLN | A | 166 | 78.472 | 215.807 | −10.023 | 0.00 | 0.00 | AAAA | H |
| ATOM | 1571 | N | LEU | A | 167 | 81.522 | 221.551 | −11.577 | 1.00 | 50.00 | AAAA | N |
| ATOM | 1572 | CA | LEU | A | 167 | 82.017 | 222.822 | −11.062 | 1.00 | 50.00 | AAAA | C |
| ATOM | 1573 | C | LEU | A | 167 | 81.174 | 223.363 | −9.927 | 1.00 | 50.00 | AAAA | C |
| ATOM | 1574 | O | LEU | A | 167 | 79.976 | 223.596 | −10.006 | 1.00 | 50.00 | AAAA | O |
| ATOM | 1575 | CB | LEU | A | 167 | 82.251 | 223.943 | −12.095 | 1.00 | 50.00 | AAAA | C |
| ATOM | 1576 | CG | LEU | A | 167 | 82.974 | 225.181 | −11.494 | 1.00 | 50.00 | AAAA | C |
| ATOM | 1577 | CD1 | LEU | A | 167 | 84.442 | 225.137 | −11.856 | 1.00 | 50.00 | AAAA | C |
| ATOM | 1578 | CD2 | LEU | A | 167 | 82.399 | 226.581 | −11.733 | 1.00 | 50.00 | AAAA | C |
| ATOM | 1579 | H | LEU | A | 167 | 80.578 | 221.470 | −11.893 | 0.00 | 0.00 | AAAA | H |
| ATOM | 1580 | N | PRO | A | 168 | 81.950 | 223.601 | −8.858 | 1.00 | 50.00 | AAAA | N |
| ATOM | 1581 | CA | PRO | A | 168 | 81.601 | 224.472 | −7.737 | 1.00 | 50.00 | AAAA | C |
| ATOM | 1582 | C | PRO | A | 168 | 80.495 | 225.511 | −7.847 | 1.00 | 50.00 | AAAA | C |
| ATOM | 1583 | O | PRO | A | 168 | 80.379 | 226.243 | −8.824 | 1.00 | 50.00 | AAAA | O |
| ATOM | 1584 | CB | PRO | A | 168 | 82.927 | 225.160 | −7.557 | 1.00 | 50.00 | AAAA | C |
| ATOM | 1585 | CG | PRO | A | 168 | 84.060 | 224.260 | −8.043 | 1.00 | 50.00 | AAAA | C |
| ATOM | 1586 | CD | PRO | A | 168 | 83.339 | 223.144 | −8.762 | 1.00 | 50.00 | AAAA | C |
| ATOM | 1587 | N | LEU | A | 169 | 79.765 | 225.601 | −6.719 | 1.00 | 50.00 | AAAA | N |
| ATOM | 1588 | CA | LEU | A | 169 | 79.010 | 226.823 | −6.457 | 1.00 | 50.00 | AAAA | C |
| ATOM | 1589 | C | LEU | A | 169 | 79.327 | 227.317 | −5.060 | 1.00 | 50.00 | AAAA | C |
| ATOM | 1590 | O | LEU | A | 169 | 78.705 | 226.945 | −4.076 | 1.00 | 50.00 | AAAA | O |
| ATOM | 1591 | CB | LEU | A | 169 | 77.509 | 226.601 | −6.664 | 1.00 | 50.00 | AAAA | C |
| ATOM | 1592 | CG | LEU | A | 169 | 76.702 | 227.900 | −6.810 | 1.00 | 50.00 | AAAA | C |
| ATOM | 1593 | CD1 | LEU | A | 169 | 75.509 | 227.692 | −7.741 | 1.00 | 50.00 | AAAA | C |
| ATOM | 1594 | CD2 | LEU | A | 169 | 76.240 | 228.506 | −5.480 | 1.00 | 50.00 | AAAA | C |
| ATOM | 1595 | H | LEU | A | 169 | 79.794 | 224.885 | −6.021 | 0.00 | 0.00 | AAAA | H |
| ATOM | 1596 | N | TYR | A | 170 | 80.365 | 228.160 | −5.029 | 1.00 | 50.00 | AAAA | N |
| ATOM | 1597 | CA | TYR | A | 170 | 80.859 | 228.618 | −3.736 | 1.00 | 50.00 | AAAA | C |
| ATOM | 1598 | C | TYR | A | 170 | 80.090 | 229.734 | −3.051 | 1.00 | 50.00 | AAAA | C |
| ATOM | 1599 | O | TYR | A | 170 | 79.387 | 230.529 | −3.664 | 1.00 | 50.00 | AAAA | O |
| ATOM | 1600 | CB | TYR | A | 170 | 82.270 | 229.118 | −3.909 | 1.00 | 50.00 | AAAA | C |
| ATOM | 1601 | CG | TYR | A | 170 | 83.246 | 228.060 | −4.351 | 1.00 | 50.00 | AAAA | C |
| ATOM | 1602 | CD1 | TYR | A | 170 | 83.786 | 227.188 | −3.385 | 1.00 | 50.00 | AAAA | C |
| ATOM | 1603 | CD2 | TYR | A | 170 | 83.663 | 228.040 | −5.696 | 1.00 | 50.00 | AAAA | C |
| ATOM | 1604 | CE1 | TYR | A | 170 | 84.804 | 226.300 | −3.772 | 1.00 | 50.00 | AAAA | C |
| ATOM | 1605 | CE2 | TYR | A | 170 | 84.675 | 227.145 | −6.068 | 1.00 | 50.00 | AAAA | C |
| ATOM | 1606 | CZ | TYR | A | 170 | 85.202 | 226.248 | −5.116 | 1.00 | 50.00 | AAAA | C |
| ATOM | 1607 | OH | TYR | A | 170 | 86.116 | 225.283 | −5.505 | 1.00 | 50.00 | AAAA | O |
| ATOM | 1608 | H | TYR | A | 170 | 80.815 | 228.443 | −5.875 | 0.00 | 0.00 | AAAA | H |
| ATOM | 1609 | HH | TYR | A | 170 | 86.203 | 225.264 | −6.449 | 0.00 | 0.00 | AAAA | H |
| ATOM | 1610 | N | GLY | A | 171 | 80.324 | 229.778 | −1.727 | 1.00 | 50.00 | AAAA | N |
| ATOM | 1611 | CA | GLY | A | 171 | 79.718 | 230.845 | −0.938 | 1.00 | 50.00 | AAAA | C |
| ATOM | 1612 | C | GLY | A | 171 | 80.556 | 231.206 | 0.266 | 1.00 | 50.00 | AAAA | C |
| ATOM | 1613 | O | GLY | A | 171 | 81.147 | 230.351 | 0.916 | 1.00 | 50.00 | AAAA | O |
| ATOM | 1614 | H | GLY | A | 171 | 80.825 | 229.026 | −1.294 | 0.00 | 0.00 | AAAA | H |
| ATOM | 1615 | N | VAL | A | 172 | 80.590 | 232.525 | 0.519 | 1.00 | 50.00 | AAAA | N |
| ATOM | 1616 | CA | VAL | A | 172 | 81.415 | 233.021 | 1.618 | 1.00 | 50.00 | AAAA | C |
| ATOM | 1617 | C | VAL | A | 172 | 80.818 | 232.725 | 2.984 | 1.00 | 50.00 | AAAA | C |
| ATOM | 1618 | O | VAL | A | 172 | 79.813 | 233.292 | 3.392 | 1.00 | 50.00 | AAAA | O |
| ATOM | 1619 | CB | VAL | A | 172 | 81.688 | 234.529 | 1.454 | 1.00 | 50.00 | AAAA | C |
| ATOM | 1620 | CG1 | VAL | A | 172 | 82.507 | 235.125 | 2.609 | 1.00 | 50.00 | AAAA | C |
| ATOM | 1621 | CG2 | VAL | A | 172 | 82.350 | 234.820 | 0.105 | 1.00 | 50.00 | AAAA | C |
| ATOM | 1622 | H | VAL | A | 172 | 80.010 | 233.146 | −0.005 | 0.00 | 0.00 | AAAA | H |
| ATOM | 1623 | N | ILE | A | 173 | 81.532 | 231.835 | 3.700 | 1.00 | 50.00 | AAAA | N |
| ATOM | 1624 | CA | ILE | A | 173 | 81.297 | 231.723 | 5.131 | 1.00 | 50.00 | AAAA | C |
| ATOM | 1625 | C | ILE | A | 173 | 81.650 | 233.024 | 5.842 | 1.00 | 50.00 | AAAA | C |
| ATOM | 1626 | O | ILE | A | 173 | 82.762 | 233.531 | 5.778 | 1.00 | 50.00 | AAAA | O |
| ATOM | 1627 | CB | ILE | A | 173 | 81.980 | 230.459 | 5.719 | 1.00 | 50.00 | AAAA | C |
| ATOM | 1628 | CG1 | ILE | A | 173 | 83.502 | 230.473 | 5.794 | 1.00 | 50.00 | AAAA | C |
| ATOM | 1629 | CG2 | ILE | A | 173 | 81.532 | 229.234 | 4.916 | 1.00 | 50.00 | AAAA | C |
| ATOM | 1630 | CD1 | ILE | A | 173 | 84.098 | 229.221 | 6.456 | 1.00 | 50.00 | AAAA | C |
| ATOM | 1631 | H | ILE | A | 173 | 82.308 | 231.363 | 3.290 | 0.00 | 0.00 | AAAA | H |
| ATOM | 1632 | N | ASP | A | 174 | 80.586 | 233.574 | 6.453 | 1.00 | 50.00 | AAAA | N |
| ATOM | 1633 | CA | ASP | A | 174 | 80.704 | 234.839 | 7.184 | 1.00 | 50.00 | AAAA | C |
| ATOM | 1634 | C | ASP | A | 174 | 81.269 | 234.616 | 8.562 | 1.00 | 50.00 | AAAA | C |
| ATOM | 1635 | O | ASP | A | 174 | 81.019 | 233.579 | 9.164 | 1.00 | 50.00 | AAAA | O |
| ATOM | 1636 | CB | ASP | A | 174 | 79.351 | 235.521 | 7.376 | 1.00 | 50.00 | AAAA | C |
| ATOM | 1637 | CG | ASP | A | 174 | 78.725 | 235.911 | 6.058 | 1.00 | 50.00 | AAAA | C |
| ATOM | 1638 | OD1 | ASP | A | 174 | 79.288 | 236.750 | 5.356 | 1.00 | 50.00 | AAAA | O |
| ATOM | 1639 | OD2 | ASP | A | 174 | 77.653 | 235.390 | 5.753 | 1.00 | 50.00 | AAAA | O |
| ATOM | 1640 | H | ASP | A | 174 | 79.712 | 233.095 | 6.406 | 0.00 | 0.00 | AAAA | H |
| ATOM | 1641 | N | THR | A | 175 | 82.056 | 235.631 | 8.994 | 1.00 | 50.00 | AAAA | N |
| ATOM | 1642 | CA | THR | A | 175 | 82.851 | 235.649 | 10.235 | 1.00 | 50.00 | AAAA | C |
| ATOM | 1643 | C | THR | A | 175 | 84.290 | 235.103 | 10.327 | 1.00 | 50.00 | AAAA | C |
| ATOM | 1644 | O | THR | A | 175 | 84.980 | 235.482 | 11.266 | 1.00 | 50.00 | AAAA | O |
| ATOM | 1645 | CB | THR | A | 175 | 82.041 | 235.308 | 11.511 | 1.00 | 50.00 | AAAA | C |
| ATOM | 1646 | CG2 | THR | A | 175 | 80.773 | 236.165 | 11.619 | 1.00 | 50.00 | AAAA | C |
| ATOM | 1647 | OG1 | THR | A | 175 | 81.720 | 233.917 | 11.598 | 1.00 | 50.00 | AAAA | O |
| ATOM | 1648 | H | THR | A | 175 | 82.129 | 236.431 | 8.398 | 0.00 | 0.00 | AAAA | H |
| ATOM | 1649 | HG1 | THR | A | 175 | 81.005 | 233.838 | 12.216 | 0.00 | 0.00 | AAAA | H |
| ATOM | 1650 | N | PRO | A | 176 | 84.776 | 234.230 | 9.390 | 1.00 | 50.00 | AAAA | N |
| ATOM | 1651 | CA | PRO | A | 176 | 86.191 | 233.854 | 9.411 | 1.00 | 50.00 | AAAA | C |
| ATOM | 1652 | C | PRO | A | 176 | 87.131 | 234.671 | 8.520 | 1.00 | 50.00 | AAAA | C |
| ATOM | 1653 | O | PRO | A | 176 | 86.851 | 234.983 | 7.367 | 1.00 | 50.00 | AAAA | O |
| ATOM | 1654 | CB | PRO | A | 176 | 86.163 | 232.371 | 9.002 | 1.00 | 50.00 | AAAA | C |
| ATOM | 1655 | CG | PRO | A | 176 | 84.693 | 232.005 | 8.826 | 1.00 | 50.00 | AAAA | C |
| ATOM | 1656 | CD | PRO | A | 176 | 84.090 | 233.345 | 8.476 | 1.00 | 50.00 | AAAA | C |
| ATOM | 1657 | N | CYS | A | 177 | 88.303 | 234.934 | 9.153 | 1.00 | 50.00 | AAAA | N |
| ATOM | 1658 | CA | CYS | A | 177 | 89.537 | 235.361 | 8.502 | 1.00 | 50.00 | AAAA | C |
| ATOM | 1659 | C | CYS | A | 177 | 90.081 | 234.117 | 7.942 | 1.00 | 50.00 | AAAA | C |
| ATOM | 1660 | O | CYS | A | 177 | 89.952 | 233.034 | 8.503 | 1.00 | 50.00 | AAAA | O |
| ATOM | 1661 | CB | CYS | A | 177 | 90.754 | 235.813 | 9.403 | 1.00 | 50.00 | AAAA | C |
| ATOM | 1662 | SG | CYS | A | 177 | 91.748 | 234.519 | 10.346 | 1.00 | 50.00 | AAAA | S |
| ATOM | 1663 | H | CYS | A | 177 | 88.399 | 234.401 | 9.975 | 0.00 | 0.00 | AAAA | H |
| ATOM | 1664 | N | TRP | A | 178 | 90.855 | 234.388 | 6.919 | 1.00 | 50.00 | AAAA | N |
| ATOM | 1665 | CA | TRP | A | 178 | 92.066 | 233.658 | 7.078 | 1.00 | 50.00 | AAAA | C |
| ATOM | 1666 | C | TRP | A | 178 | 93.221 | 234.606 | 6.981 | 1.00 | 50.00 | AAAA | C |
| ATOM | 1667 | O | TRP | A | 178 | 93.062 | 235.687 | 6.435 | 1.00 | 50.00 | AAAA | O |
| ATOM | 1668 | CB | TRP | A | 178 | 91.978 | 232.530 | 6.111 | 1.00 | 50.00 | AAAA | C |
| ATOM | 1669 | CG | TRP | A | 178 | 92.283 | 232.882 | 4.604 | 1.00 | 50.00 | AAAA | C |
| ATOM | 1670 | CD1 | TRP | A | 178 | 94.011 | 232.604 | 2.582 | 1.00 | 50.00 | AAAA | C |
| ATOM | 1671 | CD2 | TRP | A | 178 | 90.857 | 233.268 | 3.702 | 1.00 | 50.00 | AAAA | C |
| ATOM | 1672 | CE2 | TRP | A | 178 | 91.127 | 233.328 | 1.983 | 1.00 | 50.00 | AAAA | C |
| ATOM | 1673 | CE3 | TRP | A | 178 | 89.616 | 233.150 | 4.215 | 1.00 | 50.00 | AAAA | C |
| ATOM | 1674 | NE1 | TRP | A | 178 | 92.783 | 233.004 | 1.415 | 1.00 | 50.00 | AAAA | N |
| ATOM | 1675 | CZ2 | TRP | A | 178 | 90.009 | 233.330 | 1.251 | 1.00 | 50.00 | AAAA | C |
| ATOM | 1676 | CZ3 | TRP | A | 178 | 88.698 | 233.073 | 3.227 | 1.00 | 50.00 | AAAA | C |
| ATOM | 1677 | CH2 | TRP | A | 178 | 88.793 | 233.176 | 1.836 | 1.00 | 50.00 | AAAA | C |
| ATOM | 1678 | H | TRP | A | 178 | 90.817 | 235.180 | 6.312 | 0.00 | 0.00 | AAAA | H |
| ATOM | 1679 | HE1 | TRP | A | 178 | 92.959 | 233.018 | 0.450 | 0.00 | 0.00 | AAAA | H |
| ATOM | 1680 | N | LYS | A | 179 | 94.373 | 234.178 | 7.517 | 1.00 | 50.00 | AAAA | N |
| ATOM | 1681 | CA | LYS | A | 179 | 95.553 | 235.027 | 7.423 | 1.00 | 50.00 | AAAA | C |
| ATOM | 1682 | C | LYS | A | 179 | 96.769 | 234.182 | 7.123 | 1.00 | 50.00 | AAAA | C |
| ATOM | 1683 | O | LYS | A | 179 | 96.941 | 233.077 | 7.605 | 1.00 | 50.00 | AAAA | O |
| ATOM | 1684 | CB | LYS | A | 179 | 95.791 | 235.875 | 8.686 | 1.00 | 50.00 | AAAA | C |
| ATOM | 1685 | CG | LYS | A | 179 | 94.604 | 236.720 | 9.184 | 1.00 | 50.00 | AAAA | C |
| ATOM | 1686 | CD | LYS | A | 179 | 94.175 | 237.859 | 8.268 | 1.00 | 50.00 | AAAA | C |
| ATOM | 1687 | CE | LYS | A | 179 | 94.857 | 239.165 | 8.637 | 1.00 | 50.00 | AAAA | C |
| ATOM | 1688 | NZ | LYS | A | 179 | 95.258 | 239.887 | 7.420 | 1.00 | 50.00 | AAAA | N |
| ATOM | 1689 | H | LYS | A | 179 | 94.411 | 233.303 | 8.006 | 0.00 | 0.00 | AAAA | H |
| ATOM | 1690 | 1HZ | LYS | A | 179 | 96.154 | 240.379 | 7.613 | 0.00 | 0.00 | AAAA | H |
| ATOM | 1691 | 2HZ | LYS | A | 179 | 94.526 | 240.581 | 7.173 | 0.00 | 0.00 | AAAA | H |
| ATOM | 1692 | 3HZ | LYS | A | 179 | 95.406 | 239.227 | 6.628 | 0.00 | 0.00 | AAAA | H |
| ATOM | 1693 | N | LEU | A | 180 | 97.610 | 234.753 | 6.270 | 1.00 | 50.00 | AAAA | N |
| ATOM | 1694 | CA | LEU | A | 180 | 98.929 | 234.166 | 6.099 | 1.00 | 50.00 | AAAA | C |
| ATOM | 1695 | C | LEU | A | 180 | 99.901 | 234.893 | 7.001 | 1.00 | 50.00 | AAAA | C |
| ATOM | 1696 | O | LEU | A | 180 | 99.955 | 236.112 | 6.999 | 1.00 | 50.00 | AAAA | O |
| ATOM | 1697 | CB | LEU | A | 180 | 99.391 | 234.275 | 4.641 | 1.00 | 50.00 | AAAA | C |
| ATOM | 1698 | CG | LEU | A | 180 | 98.828 | 233.215 | 3.690 | 1.00 | 50.00 | AAAA | C |
| ATOM | 1699 | CD1 | LEU | A | 180 | 97.401 | 233.492 | 3.255 | 1.00 | 50.00 | AAAA | C |
| ATOM | 1700 | CD2 | LEU | A | 180 | 99.684 | 233.060 | 2.437 | 1.00 | 50.00 | AAAA | C |
| ATOM | 1701 | H | LEU | A | 180 | 97.350 | 235.618 | 5.853 | 0.00 | 0.00 | AAAA | H |
| ATOM | 1702 | N | HIS | A | 181 | 100.661 | 234.109 | 7.782 | 1.00 | 50.00 | AAAA | N |
| ATOM | 1703 | CA | HIS | A | 181 | 101.722 | 234.763 | 8.542 | 1.00 | 50.00 | AAAA | C |
| ATOM | 1704 | C | HIS | A | 181 | 103.113 | 234.458 | 7.992 | 1.00 | 50.00 | AAAA | C |
| ATOM | 1705 | O | HIS | A | 181 | 103.459 | 233.349 | 7.631 | 1.00 | 50.00 | AAAA | O |
| ATOM | 1706 | CB | HIS | A | 181 | 101.608 | 234.417 | 10.045 | 1.00 | 50.00 | AAAA | C |
| ATOM | 1707 | CG | HIS | A | 181 | 100.236 | 234.712 | 10.631 | 1.00 | 50.00 | AAAA | C |
| ATOM | 1708 | CD2 | HIS | A | 181 | 99.314 | 235.696 | 10.250 | 1.00 | 50.00 | AAAA | C |
| ATOM | 1709 | ND1 | HIS | A | 181 | 99.705 | 234.018 | 11.662 | 1.00 | 50.00 | AAAA | N |
| ATOM | 1710 | CE1 | HIS | A | 181 | 98.469 | 234.553 | 11.923 | 1.00 | 50.00 | AAAA | C |
| ATOM | 1711 | NE2 | HIS | A | 181 | 98.227 | 235.583 | 11.055 | 1.00 | 50.00 | AAAA | N |
| ATOM | 1712 | H | HIS | A | 181 | 100.530 | 233.120 | 7.813 | 0.00 | 0.00 | AAAA | H |
| ATOM | 1713 | HD1 | HIS | A | 181 | 100.126 | 233.282 | 12.154 | 0.00 | 0.00 | AAAA | H |
| ATOM | 1714 | N | THR | A | 182 | 103.927 | 235.505 | 7.920 | 1.00 | 50.00 | AAAA | N |
| ATOM | 1715 | CA | THR | A | 182 | 105.320 | 235.251 | 7.572 | 1.00 | 50.00 | AAAA | C |
| ATOM | 1716 | C | THR | A | 182 | 106.201 | 235.718 | 8.707 | 1.00 | 50.00 | AAAA | C |
| ATOM | 1717 | O | THR | A | 182 | 105.762 | 236.481 | 9.555 | 1.00 | 50.00 | AAAA | O |
| ATOM | 1718 | CB | THR | A | 182 | 105.663 | 235.940 | 6.245 | 1.00 | 50.00 | AAAA | C |
| ATOM | 1719 | CG2 | THR | A | 182 | 105.013 | 235.213 | 5.063 | 1.00 | 50.00 | AAAA | C |
| ATOM | 1720 | OG1 | THR | A | 182 | 105.237 | 237.307 | 6.257 | 1.00 | 50.00 | AAAA | O |
| ATOM | 1721 | H | THR | A | 182 | 103.618 | 236.420 | 8.144 | 0.00 | 0.00 | AAAA | H |
| ATOM | 1722 | HG1 | THR | A | 182 | 105.556 | 237.686 | 5.446 | 0.00 | 0.00 | AAAA | H |
| ATOM | 1723 | N | SER | A | 183 | 107.446 | 235.221 | 8.703 | 1.00 | 50.00 | AAAA | N |
| ATOM | 1724 | CA | SER | A | 183 | 108.341 | 235.730 | 9.731 | 1.00 | 50.00 | AAAA | C |
| ATOM | 1725 | C | SER | A | 183 | 109.718 | 236.044 | 9.184 | 1.00 | 50.00 | AAAA | C |
| ATOM | 1726 | O | SER | A | 183 | 110.465 | 235.148 | 8.805 | 1.00 | 50.00 | AAAA | O |
| ATOM | 1727 | CB | SER | A | 183 | 108.434 | 234.714 | 10.872 | 1.00 | 50.00 | AAAA | C |
| ATOM | 1728 | OG | SER | A | 183 | 109.239 | 235.207 | 11.947 | 1.00 | 50.00 | AAAA | O |
| ATOM | 1729 | H | SER | A | 183 | 107.733 | 234.488 | 8.090 | 0.00 | 0.00 | AAAA | H |
| ATOM | 1730 | HG | SER | A | 183 | 109.027 | 234.665 | 12.693 | 0.00 | 0.00 | AAAA | H |
| ATOM | 1731 | N | PRO | A | 184 | 110.055 | 237.356 | 9.180 | 1.00 | 50.00 | AAAA | N |
| ATOM | 1732 | CA | PRO | A | 184 | 111.473 | 237.707 | 9.225 | 1.00 | 50.00 | AAAA | C |
| ATOM | 1733 | C | PRO | A | 184 | 112.123 | 237.312 | 10.547 | 1.00 | 50.00 | AAAA | C |
| ATOM | 1734 | O | PRO | A | 184 | 111.497 | 236.892 | 11.509 | 1.00 | 50.00 | AAAA | O |
| ATOM | 1735 | CB | PRO | A | 184 | 111.450 | 239.227 | 9.004 | 1.00 | 50.00 | AAAA | C |
| ATOM | 1736 | CG | PRO | A | 184 | 110.083 | 239.694 | 9.495 | 1.00 | 50.00 | AAAA | C |
| ATOM | 1737 | CD | PRO | A | 184 | 109.175 | 238.521 | 9.145 | 1.00 | 50.00 | AAAA | C |
| ATOM | 1738 | N | LEU | A | 185 | 113.441 | 237.492 | 10.534 | 1.00 | 50.00 | AAAA | N |
| ATOM | 1739 | CA | LEU | A | 185 | 114.257 | 237.227 | 11.706 | 1.00 | 50.00 | AAAA | C |
| ATOM | 1740 | C | LEU | A | 185 | 114.316 | 238.372 | 12.722 | 1.00 | 50.00 | AAAA | C |
| ATOM | 1741 | O | LEU | A | 185 | 114.099 | 239.536 | 12.409 | 1.00 | 50.00 | AAAA | O |
| ATOM | 1742 | CB | LEU | A | 185 | 115.604 | 236.804 | 11.140 | 1.00 | 50.00 | AAAA | C |
| ATOM | 1743 | CG | LEU | A | 185 | 115.520 | 235.374 | 10.626 | 1.00 | 50.00 | AAAA | C |
| ATOM | 1744 | CD1 | LEU | A | 185 | 116.639 | 234.981 | 9.685 | 1.00 | 50.00 | AAAA | C |
| ATOM | 1745 | CD2 | LEU | A | 185 | 115.522 | 234.403 | 11.785 | 1.00 | 50.00 | AAAA | C |
| ATOM | 1746 | H | LEU | A | 185 | 113.881 | 237.748 | 9.678 | 0.00 | 0.00 | AAAA | H |
| ATOM | 1747 | N | CYS | A | 186 | 114.591 | 237.956 | 13.976 | 1.00 | 50.00 | AAAA | N |
| ATOM | 1748 | CA | CYS | A | 186 | 114.575 | 238.806 | 15.171 | 1.00 | 50.00 | AAAA | C |
| ATOM | 1749 | C | CYS | A | 186 | 115.862 | 238.732 | 15.970 | 1.00 | 50.00 | AAAA | C |
| ATOM | 1750 | O | CYS | A | 186 | 116.571 | 237.742 | 15.936 | 1.00 | 50.00 | AAAA | O |
| ATOM | 1751 | CB | CYS | A | 186 | 113.398 | 238.428 | 16.092 | 1.00 | 50.00 | AAAA | C |
| ATOM | 1752 | SG | CYS | A | 186 | 113.169 | 236.690 | 16.636 | 1.00 | 50.00 | AAAA | S |
| ATOM | 1753 | H | CYS | A | 186 | 114.857 | 237.005 | 14.099 | 0.00 | 0.00 | AAAA | H |
| ATOM | 1754 | N | THR | A | 187 | 116.128 | 239.805 | 16.729 | 1.00 | 50.00 | AAAA | N |
| ATOM | 1755 | CA | THR | A | 187 | 117.229 | 239.721 | 17.684 | 1.00 | 50.00 | AAAA | C |
| ATOM | 1756 | C | THR | A | 187 | 116.703 | 239.654 | 19.105 | 1.00 | 50.00 | AAAA | C |
| ATOM | 1757 | O | THR | A | 187 | 116.194 | 240.624 | 19.654 | 1.00 | 50.00 | AAAA | O |
| ATOM | 1758 | CB | THR | A | 187 | 118.175 | 240.916 | 17.505 | 1.00 | 50.00 | AAAA | C |
| ATOM | 1759 | CG2 | THR | A | 187 | 118.866 | 240.876 | 16.152 | 1.00 | 50.00 | AAAA | C |
| ATOM | 1760 | OG1 | THR | A | 187 | 117.463 | 242.152 | 17.600 | 1.00 | 50.00 | AAAA | O |
| ATOM | 1761 | H | THR | A | 187 | 115.570 | 240.629 | 16.667 | 0.00 | 0.00 | AAAA | H |
| ATOM | 1762 | HG1 | THR | A | 187 | 118.107 | 242.850 | 17.560 | 0.00 | 0.00 | AAAA | H |
| ATOM | 1763 | N | THR | A | 188 | 116.840 | 238.454 | 19.693 | 1.00 | 99.99 | AAAA | N |
| ATOM | 1764 | CA | THR | A | 188 | 116.417 | 238.316 | 21.086 | 1.00 | 99.99 | AAAA | C |
| ATOM | 1765 | C | THR | A | 188 | 117.158 | 239.266 | 22.040 | 1.00 | 99.99 | AAAA | C |
| ATOM | 1766 | O | THR | A | 188 | 118.356 | 239.490 | 21.941 | 1.00 | 99.99 | AAAA | O |
| ATOM | 1767 | CB | THR | A | 188 | 116.517 | 236.839 | 21.522 | 1.00 | 99.99 | AAAA | C |
| ATOM | 1768 | CG2 | THR | A | 188 | 115.800 | 236.545 | 22.843 | 1.00 | 99.99 | AAAA | C |
| ATOM | 1769 | OG1 | THR | A | 188 | 116.001 | 235.974 | 20.506 | 1.00 | 99.99 | AAAA | O |
| ATOM | 1770 | H | THR | A | 188 | 117.201 | 237.674 | 19.180 | 0.00 | 0.00 | AAAA | H |
| ATOM | 1771 | HG1 | THR | A | 188 | 116.085 | 235.090 | 20.839 | 0.00 | 0.00 | AAAA | H |
| ATOM | 1772 | N | ASN | A | 189 | 116.353 | 239.845 | 22.948 | 1.00 | 99.99 | AAAA | N |
| ATOM | 1773 | CA | ASN | A | 189 | 116.895 | 240.676 | 24.025 | 1.00 | 99.99 | AAAA | C |
| ATOM | 1774 | C | ASN | A | 189 | 117.315 | 239.874 | 25.240 | 1.00 | 99.99 | AAAA | C |
| ATOM | 1775 | O | ASN | A | 189 | 116.567 | 239.766 | 26.201 | 1.00 | 99.99 | AAAA | O |
| ATOM | 1776 | CB | ASN | A | 189 | 115.866 | 241.707 | 24.502 | 1.00 | 99.99 | AAAA | C |
| ATOM | 1777 | CG | ASN | A | 189 | 116.528 | 242.730 | 25.418 | 1.00 | 99.99 | AAAA | C |
| ATOM | 1778 | ND2 | ASN | A | 189 | 115.679 | 243.641 | 25.909 | 1.00 | 99.99 | AAAA | N |
| ATOM | 1779 | OD1 | ASN | A | 189 | 117.729 | 242.712 | 25.652 | 1.00 | 99.99 | AAAA | O |
| ATOM | 1780 | H | ASN | A | 189 | 115.376 | 239.665 | 22.892 | 0.00 | 0.00 | AAAA | H |
| ATOM | 1781 | 1HD2 | ASN | A | 189 | 116.025 | 244.378 | 26.492 | 0.00 | 0.00 | AAAA | H |
| ATOM | 1782 | 2HD2 | ASN | A | 189 | 114.706 | 243.607 | 25.687 | 0.00 | 0.00 | AAAA | H |
| ATOM | 1783 | N | THR | A | 190 | 118.551 | 239.370 | 25.177 | 1.00 | 99.99 | AAAA | N |
| ATOM | 1784 | CA | THR | A | 190 | 119.060 | 238.661 | 26.350 | 1.00 | 99.99 | AAAA | C |
| ATOM | 1785 | C | THR | A | 190 | 120.573 | 238.778 | 26.343 | 1.00 | 99.99 | AAAA | C |
| ATOM | 1786 | O | THR | A | 190 | 121.187 | 238.887 | 25.290 | 1.00 | 99.99 | AAAA | O |
| ATOM | 1787 | CB | THR | A | 190 | 118.600 | 237.182 | 26.339 | 1.00 | 99.99 | AAAA | C |
| ATOM | 1788 | CG2 | THR | A | 190 | 119.017 | 236.377 | 27.569 | 1.00 | 99.99 | AAAA | C |
| ATOM | 1789 | OG1 | THR | A | 190 | 117.183 | 237.087 | 26.170 | 1.00 | 99.99 | AAAA | O |
| ATOM | 1790 | H | THR | A | 190 | 119.090 | 239.454 | 24.339 | 0.00 | 0.00 | AAAA | H |
| ATOM | 1791 | HG1 | THR | A | 190 | 116.985 | 236.195 | 25.923 | 0.00 | 0.00 | AAAA | H |
| ATOM | 1792 | N | LYS | A | 191 | 121.148 | 238.747 | 27.557 | 1.00 | 99.99 | AAAA | N |
| ATOM | 1793 | CA | LYS | A | 191 | 122.608 | 238.777 | 27.693 | 1.00 | 99.99 | AAAA | C |
| ATOM | 1794 | C | LYS | A | 191 | 123.346 | 237.638 | 26.986 | 1.00 | 99.99 | AAAA | C |
| ATOM | 1795 | O | LYS | A | 191 | 124.459 | 237.782 | 26.499 | 1.00 | 99.99 | AAAA | O |
| ATOM | 1796 | CB | LYS | A | 191 | 122.971 | 238.820 | 29.183 | 1.00 | 99.99 | AAAA | C |
| ATOM | 1797 | CG | LYS | A | 191 | 122.452 | 237.604 | 29.961 | 1.00 | 99.99 | AAAA | C |
| ATOM | 1798 | CD | LYS | A | 191 | 122.831 | 237.582 | 31.439 | 1.00 | 99.99 | AAAA | C |
| ATOM | 1799 | CE | LYS | A | 191 | 122.401 | 236.276 | 32.116 | 1.00 | 99.99 | AAAA | C |
| ATOM | 1800 | NZ | LYS | A | 191 | 123.152 | 235.140 | 31.552 | 1.00 | 99.99 | AAAA | N |
| ATOM | 1801 | H | LYS | A | 191 | 120.562 | 238.720 | 28.365 | 0.00 | 0.00 | AAAA | H |
| ATOM | 1802 | 1HZ | LYS | A | 191 | 122.857 | 234.265 | 32.027 | 0.00 | 0.00 | AAAA | H |
| ATOM | 1803 | 2HZ | LYS | A | 191 | 124.170 | 235.292 | 31.699 | 0.00 | 0.00 | AAAA | H |
| ATOM | 1804 | 3HZ | LYS | A | 191 | 122.955 | 235.068 | 30.533 | 0.00 | 0.00 | AAAA | H |
| ATOM | 1805 | N | GLU | A | 192 | 122.640 | 236.490 | 26.953 | 1.00 | 99.99 | AAAA | N |
| ATOM | 1806 | CA | GLU | A | 192 | 123.162 | 235.302 | 26.280 | 1.00 | 99.99 | AAAA | C |
| ATOM | 1807 | C | GLU | A | 192 | 122.392 | 234.949 | 25.014 | 1.00 | 99.99 | AAAA | C |
| ATOM | 1808 | O | GLU | A | 192 | 122.511 | 233.860 | 24.468 | 1.00 | 99.99 | AAAA | O |
| ATOM | 1809 | CB | GLU | A | 192 | 123.160 | 234.121 | 27.254 | 1.00 | 99.99 | AAAA | C |
| ATOM | 1810 | CG | GLU | A | 192 | 121.752 | 233.794 | 27.769 | 1.00 | 99.99 | AAAA | C |
| ATOM | 1811 | CD | GLU | A | 192 | 121.775 | 232.592 | 28.686 | 1.00 | 99.99 | AAAA | C |
| ATOM | 1812 | OE1 | GLU | A | 192 | 122.306 | 232.705 | 29.790 | 1.00 | 99.99 | AAAA | O |
| ATOM | 1813 | OE2 | GLU | A | 192 | 121.248 | 231.551 | 28.298 | 1.00 | 99.99 | AAAA | O |
| ATOM | 1814 | H | GLU | A | 192 | 121.734 | 236.480 | 27.371 | 0.00 | 0.00 | AAAA | H |
| ATOM | 1815 | N | GLY | A | 193 | 121.571 | 235.927 | 24.596 | 1.00 | 99.99 | AAAA | N |
| ATOM | 1816 | CA | GLY | A | 193 | 120.762 | 235.733 | 23.399 | 1.00 | 99.99 | AAAA | C |
| ATOM | 1817 | C | GLY | A | 193 | 119.998 | 237.001 | 23.067 | 1.00 | 99.99 | AAAA | C |
| ATOM | 1818 | O | GLY | A | 193 | 118.828 | 237.078 | 23.397 | 1.00 | 99.99 | AAAA | O |
| ATOM | 1819 | H | GLY | A | 193 | 121.567 | 236.820 | 25.046 | 0.00 | 0.00 | AAAA | H |
| ATOM | 1820 | N | SER | A | 194 | 120.634 | 238.025 | 22.435 | 1.00 | 50.00 | AAAA | N |
| ATOM | 1821 | CA | SER | A | 194 | 121.932 | 238.073 | 21.756 | 1.00 | 50.00 | AAAA | C |
| ATOM | 1822 | C | SER | A | 194 | 122.069 | 237.077 | 20.606 | 1.00 | 50.00 | AAAA | C |
| ATOM | 1823 | O | SER | A | 194 | 123.150 | 236.636 | 20.245 | 1.00 | 50.00 | AAAA | O |
| ATOM | 1824 | CB | SER | A | 194 | 123.087 | 238.094 | 22.784 | 1.00 | 50.00 | AAAA | C |
| ATOM | 1825 | OG | SER | A | 194 | 124.374 | 238.179 | 22.179 | 1.00 | 50.00 | AAAA | O |
| ATOM | 1826 | H | SER | A | 194 | 120.123 | 238.884 | 22.423 | 0.00 | 0.00 | AAAA | H |
| ATOM | 1827 | HG | SER | A | 194 | 124.976 | 237.785 | 22.800 | 0.00 | 0.00 | AAAA | H |
| ATOM | 1828 | N | ASN | A | 195 | 120.890 | 236.745 | 20.032 | 1.00 | 50.00 | AAAA | N |
| ATOM | 1829 | CA | ASN | A | 195 | 120.895 | 235.785 | 18.926 | 1.00 | 50.00 | AAAA | C |
| ATOM | 1830 | C | ASN | A | 195 | 119.788 | 236.055 | 17.933 | 1.00 | 50.00 | AAAA | C |
| ATOM | 1831 | O | ASN | A | 195 | 118.719 | 236.554 | 18.258 | 1.00 | 50.00 | AAAA | O |
| ATOM | 1832 | CB | ASN | A | 195 | 120.729 | 234.328 | 19.377 | 1.00 | 50.00 | AAAA | C |
| ATOM | 1833 | CG | ASN | A | 195 | 121.903 | 233.840 | 20.197 | 1.00 | 50.00 | AAAA | C |
| ATOM | 1834 | ND2 | ASN | A | 195 | 123.070 | 233.780 | 19.536 | 1.00 | 50.00 | AAAA | N |
| ATOM | 1835 | OD1 | ASN | A | 195 | 121.757 | 233.509 | 21.364 | 1.00 | 50.00 | AAAA | O |
| ATOM | 1836 | H | ASN | A | 195 | 120.030 | 237.165 | 20.326 | 0.00 | 0.00 | AAAA | H |
| ATOM | 1837 | 1HD2 | ASN | A | 195 | 123.889 | 233.472 | 20.023 | 0.00 | 0.00 | AAAA | H |
| ATOM | 1838 | 2HD2 | ASN | A | 195 | 123.144 | 234.049 | 18.576 | 0.00 | 0.00 | AAAA | H |
| ATOM | 1839 | N | ILE | A | 196 | 120.111 | 235.652 | 16.698 | 1.00 | 50.00 | AAAA | N |
| ATOM | 1840 | CA | ILE | A | 196 | 119.126 | 235.732 | 15.626 | 1.00 | 50.00 | AAAA | C |
| ATOM | 1841 | C | ILE | A | 196 | 118.123 | 234.583 | 15.625 | 1.00 | 50.00 | AAAA | C |
| ATOM | 1842 | O | ILE | A | 196 | 118.438 | 233.440 | 15.334 | 1.00 | 50.00 | AAAA | O |
| ATOM | 1843 | CB | ILE | A | 196 | 119.858 | 235.910 | 14.285 | 1.00 | 50.00 | AAAA | C |
| ATOM | 1844 | CG1 | ILE | A | 196 | 118.914 | 236.064 | 13.104 | 1.00 | 50.00 | AAAA | C |
| ATOM | 1845 | CG2 | ILE | A | 196 | 120.924 | 234.836 | 14.033 | 1.00 | 50.00 | AAAA | C |
| ATOM | 1846 | CD1 | ILE | A | 196 | 118.089 | 237.329 | 13.295 | 1.00 | 50.00 | AAAA | C |
| ATOM | 1847 | H | ILE | A | 196 | 121.026 | 235.290 | 16.529 | 0.00 | 0.00 | AAAA | H |
| ATOM | 1848 | N | CYS | A | 197 | 116.893 | 234.971 | 15.997 | 1.00 | 50.00 | AAAA | N |
| ATOM | 1849 | CA | CYS | A | 197 | 115.751 | 234.079 | 16.173 | 1.00 | 50.00 | AAAA | C |
| ATOM | 1850 | C | CYS | A | 197 | 114.646 | 234.213 | 15.137 | 1.00 | 50.00 | AAAA | C |
| ATOM | 1851 | O | CYS | A | 197 | 114.444 | 235.261 | 14.550 | 1.00 | 50.00 | AAAA | O |
| ATOM | 1852 | CB | CYS | A | 197 | 115.161 | 234.356 | 17.548 | 1.00 | 50.00 | AAAA | C |
| ATOM | 1853 | SG | CYS | A | 197 | 114.685 | 236.091 | 17.894 | 1.00 | 50.00 | AAAA | S |
| ATOM | 1854 | H | CYS | A | 197 | 116.780 | 235.922 | 16.270 | 0.00 | 0.00 | AAAA | H |
| ATOM | 1855 | N | LEU | A | 198 | 113.899 | 233.110 | 14.958 | 1.00 | 50.00 | AAAA | N |
| ATOM | 1856 | CA | LEU | A | 198 | 112.667 | 233.216 | 14.176 | 1.00 | 50.00 | AAAA | C |
| ATOM | 1857 | C | LEU | A | 198 | 111.502 | 232.642 | 14.949 | 1.00 | 50.00 | AAAA | C |
| ATOM | 1858 | O | LEU | A | 198 | 111.573 | 231.571 | 15.536 | 1.00 | 50.00 | AAAA | O |
| ATOM | 1859 | CB | LEU | A | 198 | 112.807 | 232.519 | 12.814 | 1.00 | 50.00 | AAAA | C |
| ATOM | 1860 | CG | LEU | A | 198 | 111.736 | 232.848 | 11.764 | 1.00 | 50.00 | AAAA | C |
| ATOM | 1861 | CD1 | LEU | A | 198 | 112.351 | 233.132 | 10.395 | 1.00 | 50.00 | AAAA | C |
| ATOM | 1862 | CD2 | LEU | A | 198 | 110.691 | 231.746 | 11.640 | 1.00 | 50.00 | AAAA | C |
| ATOM | 1863 | H | LEU | A | 198 | 114.139 | 232.234 | 15.369 | 0.00 | 0.00 | AAAA | H |
| ATOM | 1864 | N | THR | A | 199 | 110.399 | 233.404 | 14.926 | 1.00 | 50.00 | AAAA | N |
| ATOM | 1865 | CA | THR | A | 199 | 109.196 | 232.868 | 15.563 | 1.00 | 50.00 | AAAA | C |
| ATOM | 1866 | C | THR | A | 199 | 108.614 | 231.657 | 14.867 | 1.00 | 50.00 | AAAA | C |
| ATOM | 1867 | O | THR | A | 199 | 108.434 | 231.604 | 13.660 | 1.00 | 50.00 | AAAA | O |
| ATOM | 1868 | CB | THR | A | 199 | 108.134 | 233.944 | 15.673 | 1.00 | 50.00 | AAAA | C |
| ATOM | 1869 | CG2 | THR | A | 199 | 108.600 | 235.073 | 16.567 | 1.00 | 50.00 | AAAA | C |
| ATOM | 1870 | OG1 | THR | A | 199 | 107.815 | 234.450 | 14.378 | 1.00 | 50.00 | AAAA | O |
| ATOM | 1871 | H | THR | A | 199 | 110.383 | 234.303 | 14.488 | 0.00 | 0.00 | AAAA | H |
| ATOM | 1872 | HG1 | THR | A | 199 | 107.058 | 235.015 | 14.467 | 0.00 | 0.00 | AAAA | H |
| ATOM | 1873 | N | ARG | A | 200 | 108.335 | 230.669 | 15.706 | 1.00 | 99.99 | AAAA | N |
| ATOM | 1874 | CA | ARG | A | 200 | 107.844 | 229.436 | 15.115 | 1.00 | 99.99 | AAAA | C |
| ATOM | 1875 | C | ARG | A | 200 | 106.378 | 229.190 | 15.411 | 1.00 | 99.99 | AAAA | C |
| ATOM | 1876 | O | ARG | A | 200 | 105.662 | 228.423 | 14.784 | 1.00 | 99.99 | AAAA | O |
| ATOM | 1877 | CB | ARG | A | 200 | 108.678 | 228.297 | 15.632 | 1.00 | 99.99 | AAAA | C |
| ATOM | 1878 | CG | ARG | A | 200 | 108.209 | 227.030 | 14.954 | 1.00 | 99.99 | AAAA | C |
| ATOM | 1879 | CD | ARG | A | 200 | 108.918 | 225.878 | 15.577 | 1.00 | 99.99 | AAAA | C |
| ATOM | 1880 | NE | ARG | A | 200 | 110.329 | 225.975 | 15.256 | 1.00 | 99.99 | AAAA | N |
| ATOM | 1881 | CZ | ARG | A | 200 | 110.750 | 225.379 | 14.129 | 1.00 | 99.99 | AAAA | C |
| ATOM | 1882 | NH1 | ARG | A | 200 | 109.923 | 224.687 | 13.346 | 1.00 | 99.99 | AAAA | N |
| ATOM | 1883 | NH2 | ARG | A | 200 | 112.018 | 225.486 | 13.796 | 1.00 | 99.99 | AAAA | N |
| ATOM | 1884 | H | ARG | A | 200 | 108.385 | 230.810 | 16.691 | 0.00 | 0.00 | AAAA | H |
| ATOM | 1885 | HE | ARG | A | 200 | 110.948 | 226.499 | 15.843 | 0.00 | 0.00 | AAAA | H |
| ATOM | 1886 | 1HH1 | ARG | A | 200 | 110.261 | 224.269 | 12.506 | 0.00 | 0.00 | AAAA | H |
| ATOM | 1887 | 2HH1 | ARG | A | 200 | 108.958 | 224.597 | 13.596 | 0.00 | 0.00 | AAAA | H |
| ATOM | 1888 | 1HH2 | ARG | A | 200 | 112.371 | 225.039 | 12.976 | 0.00 | 0.00 | AAAA | H |
| ATOM | 1889 | 2HH2 | ARG | A | 200 | 112.617 | 226.033 | 14.382 | 0.00 | 0.00 | AAAA | H |
| ATOM | 1890 | N | THR | A | 201 | 105.963 | 229.928 | 16.426 | 1.00 | 99.99 | AAAA | N |
| ATOM | 1891 | CA | THR | A | 201 | 104.593 | 229.865 | 16.882 | 1.00 | 99.99 | AAAA | C |
| ATOM | 1892 | C | THR | A | 201 | 103.612 | 230.420 | 15.819 | 1.00 | 99.99 | AAAA | C |
| ATOM | 1893 | O | THR | A | 201 | 102.458 | 230.021 | 15.744 | 1.00 | 99.99 | AAAA | O |
| ATOM | 1894 | CB | THR | A | 201 | 104.592 | 230.594 | 18.236 | 1.00 | 99.99 | AAAA | C |
| ATOM | 1895 | CG2 | THR | A | 201 | 104.442 | 229.801 | 19.526 | 1.00 | 99.99 | AAAA | C |
| ATOM | 1896 | OG1 | THR | A | 201 | 105.714 | 231.505 | 18.313 | 1.00 | 99.99 | AAAA | O |
| ATOM | 1897 | H | THR | A | 201 | 106.581 | 230.564 | 16.876 | 0.00 | 0.00 | AAAA | H |
| ATOM | 1898 | HG1 | THR | A | 201 | 105.407 | 232.228 | 18.841 | 0.00 | 0.00 | AAAA | H |
| ATOM | 1899 | N | ASP | A | 202 | 104.147 | 231.286 | 14.904 | 1.00 | 99.99 | AAAA | N |
| ATOM | 1900 | CA | ASP | A | 202 | 103.354 | 231.560 | 13.675 | 1.00 | 99.99 | AAAA | C |
| ATOM | 1901 | C | ASP | A | 202 | 103.040 | 230.384 | 12.822 | 1.00 | 99.99 | AAAA | C |
| ATOM | 1902 | O | ASP | A | 202 | 102.011 | 230.306 | 12.165 | 1.00 | 99.99 | AAAA | O |
| ATOM | 1903 | CB | ASP | A | 202 | 103.895 | 232.622 | 12.681 | 1.00 | 99.99 | AAAA | C |
| ATOM | 1904 | CG | ASP | A | 202 | 105.397 | 232.684 | 12.527 | 1.00 | 99.99 | AAAA | C |
| ATOM | 1905 | OD1 | ASP | A | 202 | 106.026 | 231.634 | 12.414 | 1.00 | 99.99 | AAAA | O |
| ATOM | 1906 | OD2 | ASP | A | 202 | 105.922 | 233.787 | 12.529 | 1.00 | 99.99 | AAAA | O |
| ATOM | 1907 | H | ASP | A | 202 | 105.016 | 231.739 | 15.096 | 0.00 | 0.00 | AAAA | H |
| ATOM | 1908 | N | ARG | A | 203 | 104.049 | 229.509 | 12.863 | 1.00 | 99.99 | AAAA | N |
| ATOM | 1909 | CA | ARG | A | 203 | 104.118 | 228.338 | 12.010 | 1.00 | 99.99 | AAAA | C |
| ATOM | 1910 | C | ARG | A | 203 | 104.060 | 228.708 | 10.542 | 1.00 | 99.99 | AAAA | C |
| ATOM | 1911 | O | ARG | A | 203 | 103.203 | 228.278 | 9.776 | 1.00 | 99.99 | AAAA | O |
| ATOM | 1912 | CB | ARG | A | 203 | 103.048 | 227.349 | 12.440 | 1.00 | 99.99 | AAAA | C |
| ATOM | 1913 | CG | ARG | A | 203 | 103.211 | 226.013 | 11.744 | 1.00 | 99.99 | AAAA | C |
| ATOM | 1914 | CD | ARG | A | 203 | 102.104 | 225.074 | 12.181 | 1.00 | 99.99 | AAAA | C |
| ATOM | 1915 | NE | ARG | A | 203 | 102.240 | 224.747 | 13.590 | 1.00 | 99.99 | AAAA | N |
| ATOM | 1916 | CZ | ARG | A | 203 | 103.043 | 223.719 | 13.917 | 1.00 | 99.99 | AAAA | C |
| ATOM | 1917 | NH1 | ARG | A | 203 | 103.680 | 223.011 | 12.986 | 1.00 | 99.99 | AAAA | N |
| ATOM | 1918 | NH2 | ARG | A | 203 | 103.197 | 223.406 | 15.191 | 1.00 | 99.99 | AAAA | N |
| ATOM | 1919 | H | ARG | A | 203 | 104.782 | 229.675 | 13.518 | 0.00 | 0.00 | AAAA | H |
| ATOM | 1920 | HE | ARG | A | 203 | 101.784 | 225.298 | 14.289 | 0.00 | 0.00 | AAAA | H |
| ATOM | 1921 | 1HH1 | ARG | A | 203 | 104.255 | 222.237 | 13.240 | 0.00 | 0.00 | AAAA | H |
| ATOM | 1922 | 2HH1 | ARG | A | 203 | 103.574 | 223.253 | 12.021 | 0.00 | 0.00 | AAAA | H |
| ATOM | 1923 | 1HH2 | ARG | A | 203 | 103.774 | 222.632 | 15.448 | 0.00 | 0.00 | AAAA | H |
| ATOM | 1924 | 2HH2 | ARG | A | 203 | 102.728 | 223.945 | 15.890 | 0.00 | 0.00 | AAAA | H |
| ATOM | 1925 | N | GLY | A | 204 | 105.011 | 229.607 | 10.203 | 1.00 | 99.99 | AAAA | N |
| ATOM | 1926 | CA | GLY | A | 204 | 104.954 | 230.214 | 8.875 | 1.00 | 99.99 | AAAA | C |
| ATOM | 1927 | C | GLY | A | 204 | 103.587 | 230.813 | 8.640 | 1.00 | 99.99 | AAAA | C |
| ATOM | 1928 | O | GLY | A | 204 | 103.047 | 231.521 | 9.487 | 1.00 | 99.99 | AAAA | O |
| ATOM | 1929 | H | GLY | A | 204 | 105.660 | 229.917 | 10.896 | 0.00 | 0.00 | AAAA | H |
| ATOM | 1930 | N | TRP | A | 205 | 103.042 | 230.439 | 7.475 | 1.00 | 99.99 | AAAA | N |
| ATOM | 1931 | CA | TRP | A | 205 | 101.686 | 230.896 | 7.246 | 1.00 | 99.99 | AAAA | C |
| ATOM | 1932 | C | TRP | A | 205 | 100.641 | 229.829 | 7.414 | 1.00 | 99.99 | AAAA | C |
| ATOM | 1933 | O | TRP | A | 205 | 100.712 | 228.796 | 6.752 | 1.00 | 99.99 | AAAA | O |
| ATOM | 1934 | CB | TRP | A | 205 | 101.552 | 231.616 | 5.893 | 1.00 | 99.99 | AAAA | C |
| ATOM | 1935 | CG | TRP | A | 205 | 101.932 | 230.783 | 4.696 | 1.00 | 99.99 | AAAA | C |
| ATOM | 1936 | CD1 | TRP | A | 205 | 101.058 | 230.078 | 3.856 | 1.00 | 99.99 | AAAA | C |
| ATOM | 1937 | CD2 | TRP | A | 205 | 103.250 | 230.557 | 4.158 | 1.00 | 99.99 | AAAA | C |
| ATOM | 1938 | CE2 | TRP | A | 205 | 103.096 | 229.716 | 3.005 | 1.00 | 99.99 | AAAA | C |
| ATOM | 1939 | CE3 | TRP | A | 205 | 104.534 | 230.991 | 4.548 | 1.00 | 99.99 | AAAA | C |
| ATOM | 1940 | NE1 | TRP | A | 205 | 101.740 | 229.453 | 2.862 | 1.00 | 99.99 | AAAA | N |
| ATOM | 1941 | CZ2 | TRP | A | 205 | 104.231 | 229.319 | 2.269 | 1.00 | 99.99 | AAAA | C |
| ATOM | 1942 | CZ3 | TRP | A | 205 | 105.662 | 230.588 | 3.803 | 1.00 | 99.99 | AAAA | C |
| ATOM | 1943 | CH2 | TRP | A | 205 | 105.511 | 229.756 | 2.671 | 1.00 | 99.99 | AAAA | C |
| ATOM | 1944 | H | TRP | A | 205 | 103.522 | 229.837 | 6.840 | 0.00 | 0.00 | AAAA | H |
| ATOM | 1945 | HE1 | TRP | A | 205 | 101.334 | 228.916 | 2.149 | 0.00 | 0.00 | AAAA | H |
| ATOM | 1946 | N | TYR | A | 206 | 99.657 | 230.194 | 8.299 | 1.00 | 50.00 | AAAA | N |
| ATOM | 1947 | CA | TYR | A | 206 | 98.205 | 230.215 | 7.991 | 1.00 | 50.00 | AAAA | C |
| ATOM | 1948 | C | TYR | A | 206 | 97.278 | 230.155 | 9.241 | 1.00 | 50.00 | AAAA | C |
| ATOM | 1949 | O | TYR | A | 206 | 97.263 | 229.115 | 9.886 | 1.00 | 50.00 | AAAA | O |
| ATOM | 1950 | CB | TYR | A | 206 | 97.919 | 229.016 | 7.083 | 1.00 | 50.00 | AAAA | C |
| ATOM | 1951 | CG | TYR | A | 206 | 97.037 | 229.151 | 5.870 | 1.00 | 50.00 | AAAA | C |
| ATOM | 1952 | CD1 | TYR | A | 206 | 97.267 | 230.032 | 4.784 | 1.00 | 50.00 | AAAA | C |
| ATOM | 1953 | CD2 | TYR | A | 206 | 95.994 | 228.220 | 5.864 | 1.00 | 50.00 | AAAA | C |
| ATOM | 1954 | CE1 | TYR | A | 206 | 96.459 | 229.898 | 3.631 | 1.00 | 50.00 | AAAA | C |
| ATOM | 1955 | CE2 | TYR | A | 206 | 95.168 | 228.119 | 4.747 | 1.00 | 50.00 | AAAA | C |
| ATOM | 1956 | CZ | TYR | A | 206 | 95.408 | 228.951 | 3.645 | 1.00 | 50.00 | AAAA | C |
| ATOM | 1957 | OH | TYR | A | 206 | 94.519 | 228.861 | 2.595 | 1.00 | 50.00 | AAAA | O |
| ATOM | 1958 | H | TYR | A | 206 | 99.991 | 230.569 | 9.167 | 0.00 | 0.00 | AAAA | H |
| ATOM | 1959 | HH | TYR | A | 206 | 93.664 | 228.646 | 2.960 | 0.00 | 0.00 | AAAA | H |
| ATOM | 1960 | N | CYS | A | 207 | 96.503 | 231.259 | 9.525 | 1.00 | 50.00 | AAAA | N |
| ATOM | 1961 | CA | CYS | A | 207 | 95.271 | 231.317 | 10.371 | 1.00 | 50.00 | AAAA | C |
| ATOM | 1962 | C | CYS | A | 207 | 94.011 | 230.928 | 9.650 | 1.00 | 50.00 | AAAA | C |
| ATOM | 1963 | O | CYS | A | 207 | 93.789 | 231.273 | 8.497 | 1.00 | 50.00 | AAAA | O |
| ATOM | 1964 | CB | CYS | A | 207 | 94.799 | 232.695 | 11.004 | 1.00 | 50.00 | AAAA | C |
| ATOM | 1965 | SG | CYS | A | 207 | 93.751 | 233.898 | 10.008 | 1.00 | 50.00 | AAAA | S |
| ATOM | 1966 | H | CYS | A | 207 | 96.847 | 232.097 | 9.128 | 0.00 | 0.00 | AAAA | H |
| ATOM | 1967 | N | ASP | A | 208 | 93.147 | 230.311 | 10.465 | 1.00 | 50.00 | AAAA | N |
| ATOM | 1968 | CA | ASP | A | 208 | 91.731 | 230.591 | 10.318 | 1.00 | 50.00 | AAAA | C |
| ATOM | 1969 | C | ASP | A | 208 | 91.256 | 231.173 | 11.629 | 1.00 | 50.00 | AAAA | C |
| ATOM | 1970 | O | ASP | A | 208 | 91.508 | 230.635 | 12.695 | 1.00 | 50.00 | AAAA | O |
| ATOM | 1971 | CB | ASP | A | 208 | 90.951 | 229.329 | 9.964 | 1.00 | 50.00 | AAAA | C |
| ATOM | 1972 | CG | ASP | A | 208 | 89.557 | 229.676 | 9.501 | 1.00 | 50.00 | AAAA | C |
| ATOM | 1973 | OD1 | ASP | A | 208 | 89.411 | 230.295 | 8.448 | 1.00 | 50.00 | AAAA | O |
| ATOM | 1974 | OD2 | ASP | A | 208 | 88.612 | 229.322 | 10.203 | 1.00 | 50.00 | AAAA | O |
| ATOM | 1975 | H | ASP | A | 208 | 93.467 | 229.778 | 11.245 | 0.00 | 0.00 | AAAA | H |
| ATOM | 1976 | N | ASN | A | 209 | 90.578 | 232.313 | 11.510 | 1.00 | 50.00 | AAAA | N |
| ATOM | 1977 | CA | ASN | A | 209 | 90.115 | 232.939 | 12.747 | 1.00 | 50.00 | AAAA | C |
| ATOM | 1978 | C | ASN | A | 209 | 88.660 | 233.282 | 12.633 | 1.00 | 50.00 | AAAA | C |
| ATOM | 1979 | O | ASN | A | 209 | 88.192 | 233.760 | 11.620 | 1.00 | 50.00 | AAAA | O |
| ATOM | 1980 | CB | ASN | A | 209 | 91.019 | 234.140 | 13.074 | 1.00 | 50.00 | AAAA | C |
| ATOM | 1981 | CG | ASN | A | 209 | 90.569 | 235.114 | 14.136 | 1.00 | 50.00 | AAAA | C |
| ATOM | 1982 | ND2 | ASN | A | 209 | 91.597 | 235.443 | 14.920 | 1.00 | 50.00 | AAAA | N |
| ATOM | 1983 | OD1 | ASN | A | 209 | 89.430 | 235.557 | 14.229 | 1.00 | 50.00 | AAAA | O |
| ATOM | 1984 | H | ASN | A | 209 | 90.483 | 232.735 | 10.605 | 0.00 | 0.00 | AAAA | H |
| ATOM | 1985 | 1HD2 | ASN | A | 209 | 91.508 | 236.011 | 15.734 | 0.00 | 0.00 | AAAA | H |
| ATOM | 1986 | 2HD2 | ASN | A | 209 | 92.505 | 235.102 | 14.681 | 0.00 | 0.00 | AAAA | H |
| ATOM | 1987 | N | ALA | A | 210 | 87.961 | 233.013 | 13.728 | 1.00 | 50.00 | AAAA | N |
| ATOM | 1988 | CA | ALA | A | 210 | 86.545 | 233.353 | 13.736 | 1.00 | 50.00 | AAAA | C |
| ATOM | 1989 | C | ALA | A | 210 | 86.131 | 233.727 | 15.141 | 1.00 | 50.00 | AAAA | C |
| ATOM | 1990 | O | ALA | A | 210 | 85.810 | 232.893 | 15.979 | 1.00 | 50.00 | AAAA | O |
| ATOM | 1991 | CB | ALA | A | 210 | 85.700 | 232.185 | 13.216 | 1.00 | 50.00 | AAAA | C |
| ATOM | 1992 | H | ALA | A | 210 | 88.423 | 232.586 | 14.505 | 0.00 | 0.00 | AAAA | H |
| ATOM | 1993 | N | GLY | A | 211 | 86.199 | 235.047 | 15.376 | 1.00 | 50.00 | AAAA | N |
| ATOM | 1994 | CA | GLY | A | 211 | 86.061 | 235.506 | 16.756 | 1.00 | 50.00 | AAAA | C |
| ATOM | 1995 | C | GLY | A | 211 | 87.336 | 235.324 | 17.562 | 1.00 | 50.00 | AAAA | C |
| ATOM | 1996 | O | GLY | A | 211 | 88.395 | 235.842 | 17.225 | 1.00 | 50.00 | AAAA | O |
| ATOM | 1997 | H | GLY | A | 211 | 86.447 | 235.664 | 14.628 | 0.00 | 0.00 | AAAA | H |
| ATOM | 1998 | N | SER | A | 212 | 87.179 | 234.545 | 18.650 | 1.00 | 50.00 | AAAA | N |
| ATOM | 1999 | CA | SER | A | 212 | 88.368 | 234.228 | 19.438 | 1.00 | 50.00 | AAAA | C |
| ATOM | 2000 | C | SER | A | 212 | 88.972 | 232.863 | 19.132 | 1.00 | 50.00 | AAAA | C |
| ATOM | 2001 | O | SER | A | 212 | 89.798 | 232.344 | 19.875 | 1.00 | 50.00 | AAAA | O |
| ATOM | 2002 | CB | SER | A | 212 | 88.057 | 234.369 | 20.936 | 1.00 | 50.00 | AAAA | C |
| ATOM | 2003 | OG | SER | A | 212 | 86.989 | 233.490 | 21.312 | 1.00 | 50.00 | AAAA | O |
| ATOM | 2004 | H | SER | A | 212 | 86.308 | 234.104 | 18.865 | 0.00 | 0.00 | AAAA | H |
| ATOM | 2005 | HG | SER | A | 212 | 86.937 | 233.502 | 22.261 | 0.00 | 0.00 | AAAA | H |
| ATOM | 2006 | N | VAL | A | 213 | 88.499 | 232.293 | 18.008 | 1.00 | 50.00 | AAAA | N |
| ATOM | 2007 | CA | VAL | A | 213 | 88.959 | 230.960 | 17.654 | 1.00 | 50.00 | AAAA | C |
| ATOM | 2008 | C | VAL | A | 213 | 90.049 | 230.996 | 16.583 | 1.00 | 50.00 | AAAA | C |
| ATOM | 2009 | O | VAL | A | 213 | 89.889 | 231.579 | 15.517 | 1.00 | 50.00 | AAAA | O |
| ATOM | 2010 | CB | VAL | A | 213 | 87.721 | 230.087 | 17.328 | 1.00 | 50.00 | AAAA | C |
| ATOM | 2011 | CG1 | VAL | A | 213 | 87.246 | 230.100 | 15.875 | 1.00 | 50.00 | AAAA | C |
| ATOM | 2012 | CG2 | VAL | A | 213 | 87.892 | 228.673 | 17.863 | 1.00 | 50.00 | AAAA | C |
| ATOM | 2013 | H | VAL | A | 213 | 87.847 | 232.774 | 17.422 | 0.00 | 0.00 | AAAA | H |
| ATOM | 2014 | N | SER | A | 214 | 91.198 | 230.394 | 16.946 | 1.00 | 50.00 | AAAA | N |
| ATOM | 2015 | CA | SER | A | 214 | 92.299 | 230.419 | 15.987 | 1.00 | 50.00 | AAAA | C |
| ATOM | 2016 | C | SER | A | 214 | 92.848 | 229.033 | 15.731 | 1.00 | 50.00 | AAAA | C |
| ATOM | 2017 | O | SER | A | 214 | 93.369 | 228.355 | 16.607 | 1.00 | 50.00 | AAAA | O |
| ATOM | 2018 | CB | SER | A | 214 | 93.418 | 231.365 | 16.433 | 1.00 | 50.00 | AAAA | C |
| ATOM | 2019 | OG | SER | A | 214 | 94.368 | 231.527 | 15.372 | 1.00 | 50.00 | AAAA | O |
| ATOM | 2020 | H | SER | A | 214 | 91.310 | 229.943 | 17.829 | 0.00 | 0.00 | AAAA | H |
| ATOM | 2021 | HG | SER | A | 214 | 95.168 | 231.845 | 15.765 | 0.00 | 0.00 | AAAA | H |
| ATOM | 2022 | N | PHE | A | 215 | 92.659 | 228.624 | 14.474 | 1.00 | 50.00 | AAAA | N |
| ATOM | 2023 | CA | PHE | A | 215 | 93.043 | 227.279 | 14.076 | 1.00 | 50.00 | AAAA | C |
| ATOM | 2024 | C | PHE | A | 215 | 94.088 | 227.363 | 12.980 | 1.00 | 50.00 | AAAA | C |
| ATOM | 2025 | O | PHE | A | 215 | 94.235 | 228.386 | 12.329 | 1.00 | 50.00 | AAAA | O |
| ATOM | 2026 | CB | PHE | A | 215 | 91.796 | 226.506 | 13.615 | 1.00 | 50.00 | AAAA | C |
| ATOM | 2027 | CG | PHE | A | 215 | 90.883 | 226.044 | 14.746 | 1.00 | 50.00 | AAAA | C |
| ATOM | 2028 | CD1 | PHE | A | 215 | 90.818 | 226.735 | 15.976 | 1.00 | 50.00 | AAAA | C |
| ATOM | 2029 | CD2 | PHE | A | 215 | 90.085 | 224.897 | 14.542 | 1.00 | 50.00 | AAAA | C |
| ATOM | 2030 | CE1 | PHE | A | 215 | 89.973 | 226.284 | 17.002 | 1.00 | 50.00 | AAAA | C |
| ATOM | 2031 | CE2 | PHE | A | 215 | 89.225 | 224.442 | 15.562 | 1.00 | 50.00 | AAAA | C |
| ATOM | 2032 | CZ | PHE | A | 215 | 89.177 | 225.144 | 16.784 | 1.00 | 50.00 | AAAA | C |
| ATOM | 2033 | H | PHE | A | 215 | 92.274 | 229.235 | 13.787 | 0.00 | 0.00 | AAAA | H |
| ATOM | 2034 | N | PHE | A | 216 | 94.815 | 226.242 | 12.825 | 1.00 | 50.00 | AAAA | N |
| ATOM | 2035 | CA | PHE | A | 216 | 95.774 | 226.106 | 11.730 | 1.00 | 50.00 | AAAA | C |
| ATOM | 2036 | C | PHE | A | 216 | 95.226 | 225.372 | 10.496 | 1.00 | 50.00 | AAAA | C |
| ATOM | 2037 | O | PHE | A | 216 | 95.184 | 224.151 | 10.447 | 1.00 | 50.00 | AAAA | O |
| ATOM | 2038 | CB | PHE | A | 216 | 96.986 | 225.375 | 12.309 | 1.00 | 50.00 | AAAA | C |
| ATOM | 2039 | CG | PHE | A | 216 | 98.120 | 225.355 | 11.325 | 1.00 | 50.00 | AAAA | C |
| ATOM | 2040 | CD1 | PHE | A | 216 | 98.789 | 226.557 | 11.011 | 1.00 | 50.00 | AAAA | C |
| ATOM | 2041 | CD2 | PHE | A | 216 | 98.477 | 224.127 | 10.736 | 1.00 | 50.00 | AAAA | C |
| ATOM | 2042 | CE1 | PHE | A | 216 | 99.822 | 226.537 | 10.058 | 1.00 | 50.00 | AAAA | C |
| ATOM | 2043 | CE2 | PHE | A | 216 | 99.516 | 224.105 | 9.795 | 1.00 | 50.00 | AAAA | C |
| ATOM | 2044 | CZ | PHE | A | 216 | 100.167 | 225.311 | 9.456 | 1.00 | 50.00 | AAAA | C |
| ATOM | 2045 | H | PHE | A | 216 | 94.712 | 225.501 | 13.488 | 0.00 | 0.00 | AAAA | H |
| ATOM | 2046 | N | PRO | A | 217 | 94.780 | 226.139 | 9.478 | 1.00 | 50.00 | AAAA | N |
| ATOM | 2047 | CA | PRO | A | 217 | 94.024 | 225.536 | 8.378 | 1.00 | 50.00 | AAAA | C |
| ATOM | 2048 | C | PRO | A | 217 | 94.754 | 225.385 | 7.042 | 1.00 | 50.00 | AAAA | C |
| ATOM | 2049 | O | PRO | A | 217 | 94.181 | 225.766 | 6.031 | 1.00 | 50.00 | AAAA | O |
| ATOM | 2050 | CB | PRO | A | 217 | 92.910 | 226.566 | 8.246 | 1.00 | 50.00 | AAAA | C |
| ATOM | 2051 | CG | PRO | A | 217 | 93.695 | 227.874 | 8.327 | 1.00 | 50.00 | AAAA | C |
| ATOM | 2052 | CD | PRO | A | 217 | 94.738 | 227.586 | 9.400 | 1.00 | 50.00 | AAAA | C |
| ATOM | 2053 | N | GLN | A | 218 | 95.989 | 224.828 | 7.042 | 1.00 | 50.00 | AAAA | N |
| ATOM | 2054 | CA | GLN | A | 218 | 96.753 | 224.680 | 5.784 | 1.00 | 50.00 | AAAA | C |
| ATOM | 2055 | C | GLN | A | 218 | 95.955 | 224.283 | 4.545 | 1.00 | 50.00 | AAAA | C |
| ATOM | 2056 | O | GLN | A | 218 | 95.597 | 223.126 | 4.368 | 1.00 | 50.00 | AAAA | O |
| ATOM | 2057 | CB | GLN | A | 218 | 97.851 | 223.635 | 5.955 | 1.00 | 50.00 | AAAA | C |
| ATOM | 2058 | CG | GLN | A | 218 | 99.215 | 224.177 | 6.367 | 1.00 | 50.00 | AAAA | C |
| ATOM | 2059 | CD | GLN | A | 218 | 100.097 | 223.017 | 6.800 | 1.00 | 50.00 | AAAA | C |
| ATOM | 2060 | NE2 | GLN | A | 218 | 101.296 | 223.397 | 7.276 | 1.00 | 50.00 | AAAA | N |
| ATOM | 2061 | OE1 | GLN | A | 218 | 99.724 | 221.854 | 6.743 | 1.00 | 50.00 | AAAA | O |
| ATOM | 2062 | H | GLN | A | 218 | 96.384 | 224.520 | 7.907 | 0.00 | 0.00 | AAAA | H |
| ATOM | 2063 | 1HE2 | GLN | A | 218 | 101.946 | 222.696 | 7.572 | 0.00 | 0.00 | AAAA | H |
| ATOM | 2064 | 2HE2 | GLN | A | 218 | 101.568 | 224.357 | 7.365 | 0.00 | 0.00 | AAAA | H |
| ATOM | 2065 | N | ALA | A | 219 | 95.684 | 225.289 | 3.693 | 1.00 | 50.00 | AAAA | N |
| ATOM | 2066 | CA | ALA | A | 219 | 94.872 | 224.945 | 2.531 | 1.00 | 50.00 | AAAA | C |
| ATOM | 2067 | C | ALA | A | 219 | 95.700 | 224.661 | 1.304 | 1.00 | 50.00 | AAAA | C |
| ATOM | 2068 | O | ALA | A | 219 | 95.655 | 225.330 | 0.277 | 1.00 | 50.00 | AAAA | O |
| ATOM | 2069 | CB | ALA | A | 219 | 93.834 | 226.007 | 2.206 | 1.00 | 50.00 | AAAA | C |
| ATOM | 2070 | H | ALA | A | 219 | 96.042 | 226.207 | 3.846 | 0.00 | 0.00 | AAAA | H |
| ATOM | 2071 | N | GLU | A | 220 | 96.452 | 223.574 | 1.498 | 1.00 | 50.00 | AAAA | N |
| ATOM | 2072 | CA | GLU | A | 220 | 97.246 | 223.004 | 0.417 | 1.00 | 50.00 | AAAA | C |
| ATOM | 2073 | C | GLU | A | 220 | 96.433 | 222.186 | −0.578 | 1.00 | 50.00 | AAAA | C |
| ATOM | 2074 | O | GLU | A | 220 | 96.892 | 221.861 | −1.665 | 1.00 | 50.00 | AAAA | O |
| ATOM | 2075 | CB | GLU | A | 220 | 98.386 | 222.168 | 1.009 | 1.00 | 50.00 | AAAA | C |
| ATOM | 2076 | CG | GLU | A | 220 | 97.917 | 221.053 | 1.955 | 1.00 | 50.00 | AAAA | C |
| ATOM | 2077 | CD | GLU | A | 220 | 99.095 | 220.214 | 2.412 | 1.00 | 50.00 | AAAA | C |
| ATOM | 2078 | OE1 | GLU | A | 220 | 99.940 | 220.729 | 3.143 | 1.00 | 50.00 | AAAA | O |
| ATOM | 2079 | OE2 | GLU | A | 220 | 99.156 | 219.047 | 2.027 | 1.00 | 50.00 | AAAA | O |
| ATOM | 2080 | H | GLU | A | 220 | 96.367 | 223.114 | 2.381 | 0.00 | 0.00 | AAAA | H |
| ATOM | 2081 | N | THR | A | 221 | 95.202 | 221.863 | −0.138 | 1.00 | 50.00 | AAAA | N |
| ATOM | 2082 | CA | THR | A | 221 | 94.305 | 221.063 | −0.970 | 1.00 | 50.00 | AAAA | C |
| ATOM | 2083 | C | THR | A | 221 | 93.220 | 221.889 | −1.639 | 1.00 | 50.00 | AAAA | C |
| ATOM | 2084 | O | THR | A | 221 | 92.285 | 221.372 | −2.241 | 1.00 | 50.00 | AAAA | O |
| ATOM | 2085 | CB | THR | A | 221 | 93.665 | 219.978 | −0.101 | 1.00 | 50.00 | AAAA | C |
| ATOM | 2086 | CG2 | THR | A | 221 | 94.713 | 219.015 | 0.462 | 1.00 | 50.00 | AAAA | C |
| ATOM | 2087 | OG1 | THR | A | 221 | 92.920 | 220.577 | 0.970 | 1.00 | 50.00 | AAAA | O |
| ATOM | 2088 | H | THR | A | 221 | 94.885 | 222.154 | 0.763 | 0.00 | 0.00 | AAAA | H |
| ATOM | 2089 | HG1 | THR | A | 221 | 92.516 | 219.856 | 1.436 | 0.00 | 0.00 | AAAA | H |
| ATOM | 2090 | N | CYS | A | 222 | 93.361 | 223.211 | −1.462 | 1.00 | 50.00 | AAAA | N |
| ATOM | 2091 | CA | CYS | A | 222 | 92.275 | 224.066 | −1.903 | 1.00 | 50.00 | AAAA | C |
| ATOM | 2092 | C | CYS | A | 222 | 92.708 | 224.982 | −3.024 | 1.00 | 50.00 | AAAA | C |
| ATOM | 2093 | O | CYS | A | 222 | 93.842 | 224.960 | −3.484 | 1.00 | 50.00 | AAAA | O |
| ATOM | 2094 | CB | CYS | A | 222 | 91.724 | 224.864 | −0.720 | 1.00 | 50.00 | AAAA | C |
| ATOM | 2095 | SG | CYS | A | 222 | 91.489 | 223.845 | 0.757 | 1.00 | 50.00 | AAAA | S |
| ATOM | 2096 | H | CYS | A | 222 | 94.178 | 223.620 | −1.056 | 0.00 | 0.00 | AAAA | H |
| ATOM | 2097 | N | LYS | A | 223 | 91.740 | 225.806 | −3.447 | 1.00 | 50.00 | AAAA | N |
| ATOM | 2098 | CA | LYS | A | 223 | 92.187 | 226.830 | −4.378 | 1.00 | 50.00 | AAAA | C |
| ATOM | 2099 | C | LYS | A | 223 | 92.291 | 228.204 | −3.765 | 1.00 | 50.00 | AAAA | C |
| ATOM | 2100 | O | LYS | A | 223 | 91.461 | 228.656 | −2.980 | 1.00 | 50.00 | AAAA | O |
| ATOM | 2101 | CB | LYS | A | 223 | 91.302 | 226.875 | −5.602 | 1.00 | 50.00 | AAAA | C |
| ATOM | 2102 | CG | LYS | A | 223 | 89.867 | 227.118 | −5.200 | 1.00 | 50.00 | AAAA | C |
| ATOM | 2103 | CD | LYS | A | 223 | 89.178 | 227.718 | −6.388 | 1.00 | 50.00 | AAAA | C |
| ATOM | 2104 | CE | LYS | A | 223 | 87.758 | 228.026 | −6.020 | 1.00 | 50.00 | AAAA | C |
| ATOM | 2105 | NZ | LYS | A | 223 | 87.615 | 229.333 | −5.390 | 1.00 | 50.00 | AAAA | N |
| ATOM | 2106 | H | LYS | A | 223 | 90.808 | 225.769 | −3.079 | 0.00 | 0.00 | AAAA | H |
| ATOM | 2107 | 1HZ | LYS | A | 223 | 86.588 | 229.460 | −5.285 | 0.00 | 0.00 | AAAA | H |
| ATOM | 2108 | 2HZ | LYS | A | 223 | 87.987 | 230.082 | −6.002 | 0.00 | 0.00 | AAAA | H |
| ATOM | 2109 | 3HZ | LYS | A | 223 | 88.064 | 229.336 | −4.453 | 0.00 | 0.00 | AAAA | H |
| ATOM | 2110 | N | VAL | A | 224 | 93.394 | 228.832 | −4.185 | 1.00 | 50.00 | AAAA | N |
| ATOM | 2111 | CA | VAL | A | 224 | 93.719 | 230.137 | −3.638 | 1.00 | 50.00 | AAAA | C |
| ATOM | 2112 | C | VAL | A | 224 | 93.665 | 231.246 | −4.687 | 1.00 | 50.00 | AAAA | C |
| ATOM | 2113 | O | VAL | A | 224 | 94.156 | 231.130 | −5.802 | 1.00 | 50.00 | AAAA | O |
| ATOM | 2114 | CB | VAL | A | 224 | 95.087 | 230.063 | −2.918 | 1.00 | 50.00 | AAAA | C |
| ATOM | 2115 | CG1 | VAL | A | 224 | 95.137 | 228.920 | −1.894 | 1.00 | 50.00 | AAAA | C |
| ATOM | 2116 | CG2 | VAL | A | 224 | 96.265 | 229.886 | −3.876 | 1.00 | 50.00 | AAAA | C |
| ATOM | 2117 | H | VAL | A | 224 | 94.040 | 228.376 | −4.795 | 0.00 | 0.00 | AAAA | H |
| ATOM | 2118 | N | GLN | A | 225 | 93.032 | 232.347 | −4.266 | 1.00 | 50.00 | AAAA | N |
| ATOM | 2119 | CA | GLN | A | 225 | 93.106 | 233.614 | −4.990 | 1.00 | 50.00 | AAAA | C |
| ATOM | 2120 | C | GLN | A | 225 | 93.503 | 234.663 | −3.968 | 1.00 | 50.00 | AAAA | C |
| ATOM | 2121 | O | GLN | A | 225 | 93.598 | 234.375 | −2.780 | 1.00 | 50.00 | AAAA | O |
| ATOM | 2122 | CB | GLN | A | 225 | 91.776 | 233.972 | −5.699 | 1.00 | 50.00 | AAAA | C |
| ATOM | 2123 | CG | GLN | A | 225 | 90.602 | 233.886 | −4.735 | 1.00 | 50.00 | AAAA | C |
| ATOM | 2124 | CD | GLN | A | 225 | 89.261 | 234.407 | −5.224 | 1.00 | 50.00 | AAAA | C |
| ATOM | 2125 | NE2 | GLN | A | 225 | 88.946 | 235.630 | −4.756 | 1.00 | 50.00 | AAAA | N |
| ATOM | 2126 | OE1 | GLN | A | 225 | 88.493 | 233.707 | −5.868 | 1.00 | 50.00 | AAAA | O |
| ATOM | 2127 | H | GLN | A | 225 | 92.638 | 232.325 | −3.353 | 0.00 | 0.00 | AAAA | H |
| ATOM | 2128 | 1HE2 | GLN | A | 225 | 88.005 | 235.960 | −4.858 | 0.00 | 0.00 | AAAA | H |
| ATOM | 2129 | 2HE2 | GLN | A | 225 | 89.574 | 236.200 | −4.230 | 0.00 | 0.00 | AAAA | H |
| ATOM | 2130 | N | SER | A | 226 | 93.728 | 235.887 | −4.474 | 1.00 | 50.00 | AAAA | N |
| ATOM | 2131 | CA | SER | A | 226 | 94.232 | 236.950 | −3.597 | 1.00 | 50.00 | AAAA | C |
| ATOM | 2132 | C | SER | A | 226 | 93.302 | 237.355 | −2.462 | 1.00 | 50.00 | AAAA | C |
| ATOM | 2133 | O | SER | A | 226 | 93.713 | 237.919 | −1.458 | 1.00 | 50.00 | AAAA | O |
| ATOM | 2134 | CB | SER | A | 226 | 94.599 | 238.177 | −4.432 | 1.00 | 50.00 | AAAA | C |
| ATOM | 2135 | OG | SER | A | 226 | 95.506 | 237.807 | −5.478 | 1.00 | 50.00 | AAAA | O |
| ATOM | 2136 | H | SER | A | 226 | 93.564 | 236.061 | −5.444 | 0.00 | 0.00 | AAAA | H |
| ATOM | 2137 | HG | SER | A | 226 | 95.748 | 238.610 | −5.923 | 0.00 | 0.00 | AAAA | H |
| ATOM | 2138 | N | ASN | A | 227 | 92.017 | 237.030 | −2.691 | 1.00 | 50.00 | AAAA | N |
| ATOM | 2139 | CA | ASN | A | 227 | 91.018 | 237.408 | −1.699 | 1.00 | 50.00 | AAAA | C |
| ATOM | 2140 | C | ASN | A | 227 | 90.308 | 236.212 | −1.071 | 1.00 | 50.00 | AAAA | C |
| ATOM | 2141 | O | ASN | A | 227 | 89.632 | 236.349 | −0.059 | 1.00 | 50.00 | AAAA | O |
| ATOM | 2142 | CB | ASN | A | 227 | 89.984 | 238.364 | −2.317 | 1.00 | 50.00 | AAAA | C |
| ATOM | 2143 | CG | ASN | A | 227 | 90.649 | 239.556 | −2.989 | 1.00 | 50.00 | AAAA | C |
| ATOM | 2144 | ND2 | ASN | A | 227 | 90.489 | 239.577 | −4.322 | 1.00 | 50.00 | AAAA | N |
| ATOM | 2145 | OD1 | ASN | A | 227 | 91.270 | 240.405 | −2.363 | 1.00 | 50.00 | AAAA | O |
| ATOM | 2146 | H | ASN | A | 227 | 91.756 | 236.538 | −3.517 | 0.00 | 0.00 | AAAA | H |
| ATOM | 2147 | 1HD2 | ASN | A | 227 | 90.906 | 240.317 | −4.850 | 0.00 | 0.00 | AAAA | H |
| ATOM | 2148 | 2HD2 | ASN | A | 227 | 89.960 | 238.883 | −4.807 | 0.00 | 0.00 | AAAA | H |
| ATOM | 2149 | N | ARG | A | 228 | 90.459 | 235.040 | −1.742 | 1.00 | 50.00 | AAAA | N |
| ATOM | 2150 | CA | ARG | A | 228 | 89.593 | 233.879 | −1.465 | 1.00 | 50.00 | AAAA | C |
| ATOM | 2151 | C | ARG | A | 228 | 90.224 | 232.447 | −1.527 | 1.00 | 50.00 | AAAA | C |
| ATOM | 2152 | O | ARG | A | 228 | 91.193 | 232.209 | −2.220 | 1.00 | 50.00 | AAAA | O |
| ATOM | 2153 | CB | ARG | A | 228 | 88.243 | 234.006 | −2.162 | 1.00 | 50.00 | AAAA | C |
| ATOM | 2154 | CG | ARG | A | 228 | 87.250 | 235.083 | −1.705 | 1.00 | 50.00 | AAAA | C |
| ATOM | 2155 | CD | ARG | A | 228 | 86.252 | 235.302 | −2.839 | 1.00 | 50.00 | AAAA | C |
| ATOM | 2156 | NE | ARG | A | 228 | 85.138 | 236.173 | −2.485 | 1.00 | 50.00 | AAAA | N |
| ATOM | 2157 | CZ | ARG | A | 228 | 83.984 | 236.104 | −3.186 | 1.00 | 50.00 | AAAA | C |
| ATOM | 2158 | NH1 | ARG | A | 228 | 83.757 | 235.153 | −4.093 | 1.00 | 50.00 | AAAA | N |
| ATOM | 2159 | NH2 | ARG | A | 228 | 83.046 | 237.014 | −2.964 | 1.00 | 50.00 | AAAA | N |
| ATOM | 2160 | H | ARG | A | 228 | 91.170 | 234.982 | −2.440 | 0.00 | 0.00 | AAAA | H |
| ATOM | 2161 | HE | ARG | A | 228 | 85.251 | 236.858 | −1.767 | 0.00 | 0.00 | AAAA | H |
| ATOM | 2162 | 1HH1 | ARG | A | 228 | 82.896 | 235.137 | −4.601 | 0.00 | 0.00 | AAAA | H |
| ATOM | 2163 | 2HH1 | ARG | A | 228 | 84.441 | 234.444 | −4.266 | 0.00 | 0.00 | AAAA | H |
| ATOM | 2164 | 1HH2 | ARG | A | 228 | 82.178 | 236.971 | −3.456 | 0.00 | 0.00 | AAAA | H |
| ATOM | 2165 | 2HH2 | ARG | A | 228 | 83.209 | 237.746 | −2.306 | 0.00 | 0.00 | AAAA | H |
| ATOM | 2166 | N | VAL | A | 229 | 89.681 | 231.500 | −0.706 | 1.00 | 50.00 | AAAA | N |
| ATOM | 2167 | CA | VAL | A | 229 | 90.170 | 230.121 | −0.502 | 1.00 | 50.00 | AAAA | C |
| ATOM | 2168 | C | VAL | A | 229 | 89.015 | 229.275 | −0.109 | 1.00 | 50.00 | AAAA | C |
| ATOM | 2169 | O | VAL | A | 229 | 88.215 | 229.554 | 0.779 | 1.00 | 50.00 | AAAA | O |
| ATOM | 2170 | CB | VAL | A | 229 | 91.255 | 229.892 | 0.565 | 1.00 | 50.00 | AAAA | C |
| ATOM | 2171 | CG1 | VAL | A | 229 | 91.309 | 228.497 | 1.226 | 1.00 | 50.00 | AAAA | C |
| ATOM | 2172 | CG2 | VAL | A | 229 | 92.608 | 230.211 | −0.030 | 1.00 | 50.00 | AAAA | C |
| ATOM | 2173 | H | VAL | A | 229 | 88.907 | 231.782 | −0.137 | 0.00 | 0.00 | AAAA | H |
| ATOM | 2174 | N | TYR | A | 230 | 88.966 | 228.245 | −0.930 | 1.00 | 50.00 | AAAA | N |
| ATOM | 2175 | CA | TYR | A | 230 | 87.728 | 227.545 | −1.088 | 1.00 | 50.00 | AAAA | C |
| ATOM | 2176 | C | TYR | A | 230 | 88.087 | 226.086 | −1.092 | 1.00 | 50.00 | AAAA | C |
| ATOM | 2177 | O | TYR | A | 230 | 88.822 | 225.610 | −1.949 | 1.00 | 50.00 | AAAA | O |
| ATOM | 2178 | CB | TYR | A | 230 | 87.167 | 227.997 | −2.425 | 1.00 | 50.00 | AAAA | C |
| ATOM | 2179 | CG | TYR | A | 230 | 86.575 | 229.400 | −2.501 | 1.00 | 50.00 | AAAA | C |
| ATOM | 2180 | CD1 | TYR | A | 230 | 86.938 | 230.467 | −1.662 | 1.00 | 50.00 | AAAA | C |
| ATOM | 2181 | CD2 | TYR | A | 230 | 85.614 | 229.609 | −3.491 | 1.00 | 50.00 | AAAA | C |
| ATOM | 2182 | CE1 | TYR | A | 230 | 86.256 | 231.687 | −1.737 | 1.00 | 50.00 | AAAA | C |
| ATOM | 2183 | CE2 | TYR | A | 230 | 84.956 | 230.835 | −3.624 | 1.00 | 50.00 | AAAA | C |
| ATOM | 2184 | CZ | TYR | A | 230 | 85.236 | 231.831 | −2.690 | 1.00 | 50.00 | AAAA | C |
| ATOM | 2185 | OH | TYR | A | 230 | 84.451 | 232.957 | −2.686 | 1.00 | 50.00 | AAAA | O |
| ATOM | 2186 | H | TYR | A | 230 | 89.724 | 228.062 | −1.555 | 0.00 | 0.00 | AAAA | H |
| ATOM | 2187 | HH | TYR | A | 230 | 83.799 | 232.903 | −3.371 | 0.00 | 0.00 | AAAA | H |
| ATOM | 2188 | N | CYS | A | 231 | 87.566 | 225.423 | −0.053 | 1.00 | 50.00 | AAAA | N |
| ATOM | 2189 | CA | CYS | A | 231 | 87.902 | 224.018 | 0.126 | 1.00 | 50.00 | AAAA | C |
| ATOM | 2190 | C | CYS | A | 231 | 86.662 | 223.160 | −0.011 | 1.00 | 50.00 | AAAA | C |
| ATOM | 2191 | O | CYS | A | 231 | 85.542 | 223.614 | 0.186 | 1.00 | 50.00 | AAAA | O |
| ATOM | 2192 | CB | CYS | A | 231 | 88.533 | 223.810 | 1.504 | 1.00 | 50.00 | AAAA | C |
| ATOM | 2193 | SG | CYS | A | 231 | 90.028 | 224.786 | 1.823 | 1.00 | 50.00 | AAAA | S |
| ATOM | 2194 | H | CYS | A | 231 | 86.886 | 225.861 | 0.533 | 0.00 | 0.00 | AAAA | H |
| ATOM | 2195 | N | ASP | A | 232 | 86.909 | 221.878 | −0.338 | 1.00 | 50.00 | AAAA | N |
| ATOM | 2196 | CA | ASP | A | 232 | 85.783 | 220.935 | −0.305 | 1.00 | 50.00 | AAAA | C |
| ATOM | 2197 | C | ASP | A | 232 | 85.291 | 220.700 | 1.112 | 1.00 | 50.00 | AAAA | C |
| ATOM | 2198 | O | ASP | A | 232 | 84.144 | 220.958 | 1.456 | 1.00 | 50.00 | AAAA | O |
| ATOM | 2199 | CB | ASP | A | 232 | 86.146 | 219.581 | −0.917 | 1.00 | 50.00 | AAAA | C |
| ATOM | 2200 | CG | ASP | A | 232 | 86.558 | 219.721 | −2.363 | 1.00 | 50.00 | AAAA | C |
| ATOM | 2201 | OD1 | ASP | A | 232 | 85.717 | 220.056 | −3.195 | 1.00 | 50.00 | AAAA | O |
| ATOM | 2202 | OD2 | ASP | A | 232 | 87.726 | 219.468 | −2.656 | 1.00 | 50.00 | AAAA | O |
| ATOM | 2203 | H | ASP | A | 232 | 87.845 | 221.587 | −0.535 | 0.00 | 0.00 | AAAA | H |
| ATOM | 2204 | N | THR | A | 233 | 86.253 | 220.221 | 1.921 | 1.00 | 50.00 | AAAA | N |
| ATOM | 2205 | CA | THR | A | 233 | 86.026 | 220.141 | 3.358 | 1.00 | 50.00 | AAAA | C |
| ATOM | 2206 | C | THR | A | 233 | 87.203 | 220.808 | 4.049 | 1.00 | 50.00 | AAAA | C |
| ATOM | 2207 | O | THR | A | 233 | 88.300 | 220.853 | 3.506 | 1.00 | 50.00 | AAAA | O |
| ATOM | 2208 | CB | THR | A | 233 | 85.872 | 218.678 | 3.815 | 1.00 | 50.00 | AAAA | C |
| ATOM | 2209 | CG2 | THR | A | 233 | 84.655 | 217.984 | 3.197 | 1.00 | 50.00 | AAAA | C |
| ATOM | 2210 | OG1 | THR | A | 233 | 87.062 | 217.931 | 3.543 | 1.00 | 50.00 | AAAA | O |
| ATOM | 2211 | H | THR | A | 233 | 87.163 | 219.982 | 1.584 | 0.00 | 0.00 | AAAA | H |
| ATOM | 2212 | HG1 | THR | A | 233 | 86.853 | 217.019 | 3.688 | 0.00 | 0.00 | AAAA | H |
| ATOM | 2213 | N | MET | A | 234 | 86.928 | 221.338 | 5.257 | 1.00 | 50.00 | AAAA | N |
| ATOM | 2214 | CA | MET | A | 234 | 88.021 | 221.983 | 5.991 | 1.00 | 50.00 | AAAA | C |
| ATOM | 2215 | C | MET | A | 234 | 89.162 | 221.062 | 6.420 | 1.00 | 50.00 | AAAA | C |
| ATOM | 2216 | O | MET | A | 234 | 88.955 | 219.928 | 6.838 | 1.00 | 50.00 | AAAA | O |
| ATOM | 2217 | CB | MET | A | 234 | 87.464 | 222.759 | 7.196 | 1.00 | 50.00 | AAAA | C |
| ATOM | 2218 | CG | MET | A | 234 | 86.769 | 221.878 | 8.245 | 1.00 | 50.00 | AAAA | C |
| ATOM | 2219 | SD | MET | A | 234 | 86.113 | 222.792 | 9.653 | 1.00 | 50.00 | AAAA | S |
| ATOM | 2220 | CE | MET | A | 234 | 84.759 | 223.629 | 8.813 | 1.00 | 50.00 | AAAA | C |
| ATOM | 2221 | H | MET | A | 234 | 86.005 | 221.324 | 5.635 | 0.00 | 0.00 | AAAA | H |
| ATOM | 2222 | N | ASN | A | 235 | 90.380 | 221.633 | 6.303 | 1.00 | 50.00 | AAAA | N |
| ATOM | 2223 | CA | ASN | A | 235 | 91.593 | 220.901 | 6.696 | 1.00 | 50.00 | AAAA | C |
| ATOM | 2224 | C | ASN | A | 235 | 92.261 | 221.520 | 7.921 | 1.00 | 50.00 | AAAA | C |
| ATOM | 2225 | O | ASN | A | 235 | 93.477 | 221.568 | 8.072 | 1.00 | 50.00 | AAAA | O |
| ATOM | 2226 | CB | ASN | A | 235 | 92.553 | 220.815 | 5.496 | 1.00 | 50.00 | AAAA | C |
| ATOM | 2227 | CG | ASN | A | 235 | 93.724 | 219.886 | 5.788 | 1.00 | 50.00 | AAAA | C |
| ATOM | 2228 | ND2 | ASN | A | 235 | 94.921 | 220.488 | 5.671 | 1.00 | 50.00 | AAAA | N |
| ATOM | 2229 | OD1 | ASN | A | 235 | 93.567 | 218.716 | 6.114 | 1.00 | 50.00 | AAAA | O |
| ATOM | 2230 | H | ASN | A | 235 | 90.444 | 222.565 | 5.948 | 0.00 | 0.00 | AAAA | H |
| ATOM | 2231 | 1HD2 | ASN | A | 235 | 95.760 | 219.979 | 5.854 | 0.00 | 0.00 | AAAA | H |
| ATOM | 2232 | 2HD2 | ASN | A | 235 | 94.978 | 221.454 | 5.417 | 0.00 | 0.00 | AAAA | H |
| ATOM | 2233 | N | SER | A | 236 | 91.376 | 222.026 | 8.795 | 1.00 | 50.00 | AAAA | N |
| ATOM | 2234 | CA | SER | A | 236 | 91.879 | 222.629 | 10.023 | 1.00 | 50.00 | AAAA | C |
| ATOM | 2235 | C | SER | A | 236 | 92.589 | 221.689 | 10.987 | 1.00 | 50.00 | AAAA | C |
| ATOM | 2236 | O | SER | A | 236 | 92.327 | 220.494 | 11.060 | 1.00 | 50.00 | AAAA | O |
| ATOM | 2237 | CB | SER | A | 236 | 90.731 | 223.367 | 10.705 | 1.00 | 50.00 | AAAA | C |
| ATOM | 2238 | OG | SER | A | 236 | 91.232 | 224.093 | 11.822 | 1.00 | 50.00 | AAAA | O |
| ATOM | 2239 | H | SER | A | 236 | 90.399 | 221.959 | 8.602 | 0.00 | 0.00 | AAAA | H |
| ATOM | 2240 | HG | SER | A | 236 | 90.509 | 224.593 | 12.175 | 0.00 | 0.00 | AAAA | H |
| ATOM | 2241 | N | LEU | A | 237 | 93.512 | 222.329 | 11.730 | 1.00 | 50.00 | AAAA | N |
| ATOM | 2242 | CA | LEU | A | 237 | 94.269 | 221.640 | 12.768 | 1.00 | 50.00 | AAAA | C |
| ATOM | 2243 | C | LEU | A | 237 | 94.205 | 222.440 | 14.055 | 1.00 | 50.00 | AAAA | C |
| ATOM | 2244 | O | LEU | A | 237 | 94.246 | 223.665 | 14.045 | 1.00 | 50.00 | AAAA | O |
| ATOM | 2245 | CB | LEU | A | 237 | 95.734 | 221.465 | 12.353 | 1.00 | 50.00 | AAAA | C |
| ATOM | 2246 | CG | LEU | A | 237 | 95.934 | 220.616 | 11.092 | 1.00 | 50.00 | AAAA | C |
| ATOM | 2247 | CD1 | LEU | A | 237 | 97.380 | 220.664 | 10.595 | 1.00 | 50.00 | AAAA | C |
| ATOM | 2248 | CD2 | LEU | A | 237 | 95.458 | 219.175 | 11.287 | 1.00 | 50.00 | AAAA | C |
| ATOM | 2249 | H | LEU | A | 237 | 93.646 | 223.313 | 11.613 | 0.00 | 0.00 | AAAA | H |
| ATOM | 2250 | N | THR | A | 238 | 94.099 | 221.692 | 15.167 | 1.00 | 50.00 | AAAA | N |
| ATOM | 2251 | CA | THR | A | 238 | 94.054 | 222.399 | 16.446 | 1.00 | 50.00 | AAAA | C |
| ATOM | 2252 | C | THR | A | 238 | 95.421 | 222.863 | 16.920 | 1.00 | 50.00 | AAAA | C |
| ATOM | 2253 | O | THR | A | 238 | 96.440 | 222.194 | 16.803 | 1.00 | 50.00 | AAAA | O |
| ATOM | 2254 | CB | THR | A | 238 | 93.335 | 221.544 | 17.508 | 1.00 | 50.00 | AAAA | C |
| ATOM | 2255 | CG2 | THR | A | 238 | 93.195 | 222.219 | 18.879 | 1.00 | 50.00 | AAAA | C |
| ATOM | 2256 | OG1 | THR | A | 238 | 92.033 | 221.188 | 17.030 | 1.00 | 50.00 | AAAA | O |
| ATOM | 2257 | H | THR | A | 238 | 94.104 | 220.696 | 15.117 | 0.00 | 0.00 | AAAA | H |
| ATOM | 2258 | HG1 | THR | A | 238 | 91.602 | 220.734 | 17.743 | 0.00 | 0.00 | AAAA | H |
| ATOM | 2259 | N | LEU | A | 239 | 95.371 | 224.087 | 17.459 | 1.00 | 50.00 | AAAA | N |
| ATOM | 2260 | CA | LEU | A | 239 | 96.562 | 224.689 | 18.033 | 1.00 | 50.00 | AAAA | C |
| ATOM | 2261 | C | LEU | A | 239 | 96.604 | 224.511 | 19.548 | 1.00 | 50.00 | AAAA | C |
| ATOM | 2262 | O | LEU | A | 239 | 95.580 | 224.619 | 20.207 | 1.00 | 50.00 | AAAA | O |
| ATOM | 2263 | CB | LEU | A | 239 | 96.515 | 226.166 | 17.651 | 1.00 | 50.00 | AAAA | C |
| ATOM | 2264 | CG | LEU | A | 239 | 96.708 | 226.452 | 16.163 | 1.00 | 50.00 | AAAA | C |
| ATOM | 2265 | CD1 | LEU | A | 239 | 96.502 | 227.933 | 15.846 | 1.00 | 50.00 | AAAA | C |
| ATOM | 2266 | CD2 | LEU | A | 239 | 98.080 | 225.979 | 15.682 | 1.00 | 50.00 | AAAA | C |
| ATOM | 2267 | H | LEU | A | 239 | 94.510 | 224.592 | 17.511 | 0.00 | 0.00 | AAAA | H |
| ATOM | 2268 | N | PRO | A | 240 | 97.826 | 224.252 | 20.091 | 1.00 | 50.00 | AAAA | N |
| ATOM | 2269 | CA | PRO | A | 240 | 97.990 | 224.268 | 21.552 | 1.00 | 50.00 | AAAA | C |
| ATOM | 2270 | C | PRO | A | 240 | 97.733 | 225.613 | 22.220 | 1.00 | 50.00 | AAAA | C |
| ATOM | 2271 | O | PRO | A | 240 | 97.734 | 226.674 | 21.603 | 1.00 | 50.00 | AAAA | O |
| ATOM | 2272 | CB | PRO | A | 240 | 99.436 | 223.786 | 21.746 | 1.00 | 50.00 | AAAA | C |
| ATOM | 2273 | CG | PRO | A | 240 | 100.157 | 224.093 | 20.434 | 1.00 | 50.00 | AAAA | C |
| ATOM | 2274 | CD | PRO | A | 240 | 99.060 | 223.893 | 19.391 | 1.00 | 50.00 | AAAA | C |
| ATOM | 2275 | N | SER | A | 241 | 97.536 | 225.500 | 23.551 | 1.00 | 50.00 | AAAA | N |
| ATOM | 2276 | CA | SER | A | 241 | 97.231 | 226.688 | 24.342 | 1.00 | 50.00 | AAAA | C |
| ATOM | 2277 | C | SER | A | 241 | 98.300 | 227.761 | 24.266 | 1.00 | 50.00 | AAAA | C |
| ATOM | 2278 | O | SER | A | 241 | 98.004 | 228.934 | 24.117 | 1.00 | 50.00 | AAAA | O |
| ATOM | 2279 | CB | SER | A | 241 | 96.957 | 226.317 | 25.803 | 1.00 | 50.00 | AAAA | C |
| ATOM | 2280 | OG | SER | A | 241 | 95.882 | 225.373 | 25.876 | 1.00 | 50.00 | AAAA | O |
| ATOM | 2281 | H | SER | A | 241 | 97.581 | 224.608 | 23.999 | 0.00 | 0.00 | AAAA | H |
| ATOM | 2282 | HG | SER | A | 241 | 95.716 | 225.217 | 26.798 | 0.00 | 0.00 | AAAA | H |
| ATOM | 2283 | N | GLU | A | 242 | 99.565 | 227.300 | 24.331 | 1.00 | 50.00 | AAAA | N |
| ATOM | 2284 | CA | GLU | A | 242 | 100.666 | 228.260 | 24.241 | 1.00 | 50.00 | AAAA | C |
| ATOM | 2285 | C | GLU | A | 242 | 100.708 | 229.066 | 22.954 | 1.00 | 50.00 | AAAA | C |
| ATOM | 2286 | O | GLU | A | 242 | 100.845 | 230.281 | 22.978 | 1.00 | 50.00 | AAAA | O |
| ATOM | 2287 | CB | GLU | A | 242 | 102.007 | 227.574 | 24.459 | 1.00 | 50.00 | AAAA | C |
| ATOM | 2288 | CG | GLU | A | 242 | 102.129 | 226.946 | 25.848 | 1.00 | 50.00 | AAAA | C |
| ATOM | 2289 | CD | GLU | A | 242 | 103.485 | 226.285 | 25.966 | 1.00 | 50.00 | AAAA | C |
| ATOM | 2290 | OE1 | GLU | A | 242 | 103.739 | 225.335 | 25.225 | 1.00 | 50.00 | AAAA | O |
| ATOM | 2291 | OE2 | GLU | A | 242 | 104.286 | 226.729 | 26.789 | 1.00 | 50.00 | AAAA | O |
| ATOM | 2292 | H | GLU | A | 242 | 99.729 | 226.318 | 24.417 | 0.00 | 0.00 | AAAA | H |
| ATOM | 2293 | N | VAL | A | 243 | 100.540 | 228.341 | 21.830 | 1.00 | 50.00 | AAAA | N |
| ATOM | 2294 | CA | VAL | A | 243 | 100.519 | 229.063 | 20.551 | 1.00 | 50.00 | AAAA | C |
| ATOM | 2295 | C | VAL | A | 243 | 99.311 | 229.990 | 20.356 | 1.00 | 50.00 | AAAA | C |
| ATOM | 2296 | O | VAL | A | 243 | 99.383 | 231.011 | 19.684 | 1.00 | 50.00 | AAAA | O |
| ATOM | 2297 | CB | VAL | A | 243 | 100.675 | 228.070 | 19.384 | 1.00 | 50.00 | AAAA | C |
| ATOM | 2298 | CG1 | VAL | A | 243 | 99.381 | 227.352 | 19.075 | 1.00 | 50.00 | AAAA | C |
| ATOM | 2299 | CG2 | VAL | A | 243 | 101.148 | 228.718 | 18.098 | 1.00 | 50.00 | AAAA | C |
| ATOM | 2300 | H | VAL | A | 243 | 100.459 | 227.344 | 21.865 | 0.00 | 0.00 | AAAA | H |
| ATOM | 2301 | N | ASN | A | 244 | 98.195 | 229.575 | 21.001 | 1.00 | 50.00 | AAAA | N |
| ATOM | 2302 | CA | ASN | A | 244 | 96.968 | 230.371 | 20.919 | 1.00 | 50.00 | AAAA | C |
| ATOM | 2303 | C | ASN | A | 244 | 96.998 | 231.605 | 21.793 | 1.00 | 50.00 | AAAA | C |
| ATOM | 2304 | O | ASN | A | 244 | 96.359 | 232.609 | 21.506 | 1.00 | 50.00 | AAAA | O |
| ATOM | 2305 | CB | ASN | A | 244 | 95.724 | 229.578 | 21.325 | 1.00 | 50.00 | AAAA | C |
| ATOM | 2306 | CG | ASN | A | 244 | 95.356 | 228.519 | 20.311 | 1.00 | 50.00 | AAAA | C |
| ATOM | 2307 | ND2 | ASN | A | 244 | 95.065 | 228.987 | 19.089 | 1.00 | 50.00 | AAAA | N |
| ATOM | 2308 | OD1 | ASN | A | 244 | 95.309 | 227.338 | 20.612 | 1.00 | 50.00 | AAAA | O |
| ATOM | 2309 | H | ASN | A | 244 | 98.233 | 228.744 | 21.553 | 0.00 | 0.00 | AAAA | H |
| ATOM | 2310 | 1HD2 | ASN | A | 244 | 94.784 | 228.334 | 18.384 | 0.00 | 0.00 | AAAA | H |
| ATOM | 2311 | 2HD2 | ASN | A | 244 | 95.099 | 229.953 | 18.841 | 0.00 | 0.00 | AAAA | H |
| ATOM | 2312 | N | LEU | A | 245 | 97.793 | 231.456 | 22.872 | 1.00 | 50.00 | AAAA | N |
| ATOM | 2313 | CA | LEU | A | 245 | 98.099 | 232.542 | 23.797 | 1.00 | 50.00 | AAAA | C |
| ATOM | 2314 | C | LEU | A | 245 | 99.071 | 233.558 | 23.230 | 1.00 | 50.00 | AAAA | C |
| ATOM | 2315 | O | LEU | A | 245 | 100.142 | 233.762 | 23.779 | 1.00 | 50.00 | AAAA | O |
| ATOM | 2316 | CB | LEU | A | 245 | 98.729 | 231.997 | 25.085 | 1.00 | 50.00 | AAAA | C |
| ATOM | 2317 | CG | LEU | A | 245 | 97.822 | 231.208 | 26.027 | 1.00 | 50.00 | AAAA | C |
| ATOM | 2318 | CD1 | LEU | A | 245 | 98.620 | 230.505 | 27.128 | 1.00 | 50.00 | AAAA | C |
| ATOM | 2319 | CD2 | LEU | A | 245 | 96.683 | 232.066 | 26.570 | 1.00 | 50.00 | AAAA | C |
| ATOM | 2320 | H | LEU | A | 245 | 98.222 | 230.566 | 23.004 | 0.00 | 0.00 | AAAA | H |
| ATOM | 2321 | N | CYS | A | 246 | 98.657 | 234.213 | 22.140 | 1.00 | 50.00 | AAAA | N |
| ATOM | 2322 | CA | CYS | A | 246 | 99.384 | 235.430 | 21.797 | 1.00 | 50.00 | AAAA | C |
| ATOM | 2323 | C | CYS | A | 246 | 98.538 | 236.367 | 21.028 | 1.00 | 50.00 | AAAA | C |
| ATOM | 2324 | O | CYS | A | 246 | 97.424 | 236.168 | 20.555 | 1.00 | 50.00 | AAAA | O |
| ATOM | 2325 | CB | CYS | A | 246 | 100.658 | 235.312 | 20.930 | 1.00 | 50.00 | AAAA | C |
| ATOM | 2326 | SG | CYS | A | 246 | 102.286 | 236.350 | 21.138 | 1.00 | 50.00 | AAAA | S |
| ATOM | 2327 | H | CYS | A | 246 | 97.779 | 233.993 | 21.714 | 0.00 | 0.00 | AAAA | H |
| ATOM | 2328 | N | ASN | A | 247 | 99.242 | 237.468 | 20.947 | 1.00 | 99.99 | AAAA | N |
| ATOM | 2329 | CA | ASN | A | 247 | 98.692 | 238.685 | 20.481 | 1.00 | 99.99 | AAAA | C |
| ATOM | 2330 | C | ASN | A | 247 | 99.390 | 238.841 | 19.142 | 1.00 | 99.99 | AAAA | C |
| ATOM | 2331 | O | ASN | A | 247 | 100.496 | 239.367 | 19.120 | 1.00 | 99.99 | AAAA | O |
| ATOM | 2332 | CB | ASN | A | 247 | 99.115 | 239.619 | 21.631 | 1.00 | 99.99 | AAAA | C |
| ATOM | 2333 | CG | ASN | A | 247 | 98.386 | 239.233 | 22.918 | 1.00 | 99.99 | AAAA | C |
| ATOM | 2334 | ND2 | ASN | A | 247 | 99.151 | 239.194 | 24.028 | 1.00 | 99.99 | AAAA | N |
| ATOM | 2335 | OD1 | ASN | A | 247 | 97.191 | 238.972 | 22.902 | 1.00 | 99.99 | AAAA | O |
| ATOM | 2336 | H | ASN | A | 247 | 100.197 | 237.524 | 21.233 | 0.00 | 0.00 | AAAA | H |
| ATOM | 2337 | 1HD2 | ASN | A | 247 | 98.730 | 238.924 | 24.896 | 0.00 | 0.00 | AAAA | H |
| ATOM | 2338 | 2HD2 | ASN | A | 247 | 100.126 | 239.413 | 24.040 | 0.00 | 0.00 | AAAA | H |
| ATOM | 2339 | N | VAL | A | 248 | 98.715 | 238.320 | 18.062 | 1.00 | 50.00 | AAAA | N |
| ATOM | 2340 | CA | VAL | A | 248 | 99.253 | 238.330 | 16.679 | 1.00 | 50.00 | AAAA | C |
| ATOM | 2341 | C | VAL | A | 248 | 99.138 | 239.633 | 15.946 | 1.00 | 50.00 | AAAA | C |
| ATOM | 2342 | O | VAL | A | 248 | 98.357 | 239.897 | 15.045 | 1.00 | 50.00 | AAAA | O |
| ATOM | 2343 | CB | VAL | A | 248 | 98.773 | 237.242 | 15.698 | 1.00 | 50.00 | AAAA | C |
| ATOM | 2344 | CG1 | VAL | A | 248 | 99.529 | 237.267 | 14.344 | 1.00 | 50.00 | AAAA | C |
| ATOM | 2345 | CG2 | VAL | A | 248 | 98.741 | 235.849 | 16.314 | 1.00 | 50.00 | AAAA | C |
| ATOM | 2346 | H | VAL | A | 248 | 97.812 | 237.942 | 18.234 | 0.00 | 0.00 | AAAA | H |
| ATOM | 2347 | N | ASP | A | 249 | 100.001 | 240.467 | 16.428 | 1.00 | 50.00 | AAAA | N |
| ATOM | 2348 | CA | ASP | A | 249 | 99.814 | 241.831 | 16.183 | 1.00 | 50.00 | AAAA | C |
| ATOM | 2349 | C | ASP | A | 249 | 100.637 | 242.744 | 14.881 | 1.00 | 50.00 | AAAA | C |
| ATOM | 2350 | O | ASP | A | 249 | 101.333 | 242.064 | 14.143 | 1.00 | 50.00 | AAAA | O |
| ATOM | 2351 | CB | ASP | A | 249 | 99.438 | 241.807 | 17.873 | 1.00 | 50.00 | AAAA | C |
| ATOM | 2352 | CG | ASP | A | 249 | 99.848 | 242.249 | 19.362 | 1.00 | 50.00 | AAAA | C |
| ATOM | 2353 | OD1 | ASP | A | 249 | 98.948 | 242.311 | 20.189 | 1.00 | 50.00 | AAAA | O |
| ATOM | 2354 | OD2 | ASP | A | 249 | 100.964 | 242.422 | 19.811 | 1.00 | 50.00 | AAAA | O |
| ATOM | 2355 | H | ASP | A | 249 | 100.545 | 240.202 | 17.230 | 0.00 | 0.00 | AAAA | H |
| ATOM | 2356 | N | ILE | A | 250 | 100.622 | 244.208 | 14.619 | 1.00 | 50.00 | AAAA | N |
| ATOM | 2357 | CA | ILE | A | 250 | 100.849 | 245.390 | 13.673 | 1.00 | 50.00 | AAAA | C |
| ATOM | 2358 | C | ILE | A | 250 | 102.154 | 246.106 | 13.994 | 1.00 | 50.00 | AAAA | C |
| ATOM | 2359 | O | ILE | A | 250 | 102.876 | 246.623 | 13.149 | 1.00 | 50.00 | AAAA | O |
| ATOM | 2360 | CB | ILE | A | 250 | 99.798 | 246.632 | 13.618 | 1.00 | 50.00 | AAAA | C |
| ATOM | 2361 | CG1 | ILE | A | 250 | 98.253 | 246.573 | 13.560 | 1.00 | 50.00 | AAAA | C |
| ATOM | 2362 | CG2 | ILE | A | 250 | 100.093 | 247.632 | 12.491 | 1.00 | 50.00 | AAAA | C |
| ATOM | 2363 | CD1 | ILE | A | 250 | 97.363 | 247.817 | 13.401 | 1.00 | 50.00 | AAAA | C |
| ATOM | 2364 | H | ILE | A | 250 | 100.045 | 244.799 | 15.156 | 0.00 | 0.00 | AAAA | H |
| ATOM | 2365 | N | PHE | A | 251 | 102.422 | 246.102 | 15.302 | 1.00 | 50.00 | AAAA | N |
| ATOM | 2366 | CA | PHE | A | 251 | 103.763 | 246.418 | 15.739 | 1.00 | 50.00 | AAAA | C |
| ATOM | 2367 | C | PHE | A | 251 | 104.619 | 245.318 | 16.379 | 1.00 | 50.00 | AAAA | C |
| ATOM | 2368 | O | PHE | A | 251 | 105.520 | 244.955 | 15.656 | 1.00 | 50.00 | AAAA | O |
| ATOM | 2369 | CB | PHE | A | 251 | 103.837 | 247.793 | 16.415 | 1.00 | 50.00 | AAAA | C |
| ATOM | 2370 | CG | PHE | A | 251 | 105.260 | 248.249 | 16.276 | 1.00 | 50.00 | AAAA | C |
| ATOM | 2371 | CD1 | PHE | A | 251 | 105.780 | 248.491 | 14.988 | 1.00 | 50.00 | AAAA | C |
| ATOM | 2372 | CD2 | PHE | A | 251 | 106.059 | 248.352 | 17.432 | 1.00 | 50.00 | AAAA | C |
| ATOM | 2373 | CE1 | PHE | A | 251 | 107.152 | 248.742 | 14.863 | 1.00 | 50.00 | AAAA | C |
| ATOM | 2374 | CE2 | PHE | A | 251 | 107.434 | 248.618 | 17.296 | 1.00 | 50.00 | AAAA | C |
| ATOM | 2375 | CZ | PHE | A | 251 | 107.975 | 248.775 | 16.006 | 1.00 | 50.00 | AAAA | C |
| ATOM | 2376 | H | PHE | A | 251 | 101.682 | 245.921 | 15.932 | 0.00 | 0.00 | AAAA | H |
| ATOM | 2377 | N | ASN | A | 252 | 104.340 | 244.834 | 17.647 | 1.00 | 50.00 | AAAA | N |
| ATOM | 2378 | CA | ASN | A | 252 | 105.033 | 243.882 | 18.614 | 1.00 | 50.00 | AAAA | C |
| ATOM | 2379 | C | ASN | A | 252 | 104.384 | 242.591 | 19.293 | 1.00 | 50.00 | AAAA | C |
| ATOM | 2380 | O | ASN | A | 252 | 103.512 | 242.707 | 20.139 | 1.00 | 50.00 | AAAA | O |
| ATOM | 2381 | CB | ASN | A | 252 | 105.540 | 244.690 | 19.819 | 1.00 | 50.00 | AAAA | C |
| ATOM | 2382 | CG | ASN | A | 252 | 106.547 | 243.855 | 20.597 | 1.00 | 50.00 | AAAA | C |
| ATOM | 2383 | ND2 | ASN | A | 252 | 106.623 | 244.148 | 21.893 | 1.00 | 50.00 | AAAA | N |
| ATOM | 2384 | OD1 | ASN | A | 252 | 107.175 | 242.946 | 20.071 | 1.00 | 50.00 | AAAA | O |
| ATOM | 2385 | H | ASN | A | 252 | 103.482 | 245.250 | 17.940 | 0.00 | 0.00 | AAAA | H |
| ATOM | 2386 | 1HD2 | ASN | A | 252 | 107.147 | 243.493 | 22.433 | 0.00 | 0.00 | AAAA | H |
| ATOM | 2387 | 2HD2 | ASN | A | 252 | 106.180 | 244.935 | 22.318 | 0.00 | 0.00 | AAAA | H |
| ATOM | 2388 | N | PRO | A | 253 | 104.898 | 241.352 | 19.011 | 1.00 | 99.99 | AAAA | N |
| ATOM | 2389 | CA | PRO | A | 253 | 104.423 | 240.065 | 19.549 | 1.00 | 99.99 | AAAA | C |
| ATOM | 2390 | C | PRO | A | 253 | 104.538 | 239.774 | 20.997 | 1.00 | 99.99 | AAAA | C |
| ATOM | 2391 | O | PRO | A | 253 | 105.536 | 240.132 | 21.600 | 1.00 | 99.99 | AAAA | O |
| ATOM | 2392 | CB | PRO | A | 253 | 105.360 | 239.020 | 18.964 | 1.00 | 99.99 | AAAA | C |
| ATOM | 2393 | CG | PRO | A | 253 | 105.783 | 239.605 | 17.669 | 1.00 | 99.99 | AAAA | C |
| ATOM | 2394 | CD | PRO | A | 253 | 105.955 | 241.057 | 18.095 | 1.00 | 99.99 | AAAA | C |
| ATOM | 2395 | N | LYS | A | 254 | 103.550 | 238.969 | 21.474 | 1.00 | 99.99 | AAAA | N |
| ATOM | 2396 | CA | LYS | A | 254 | 103.746 | 238.467 | 22.842 | 1.00 | 99.99 | AAAA | C |
| ATOM | 2397 | C | LYS | A | 254 | 102.900 | 237.272 | 23.360 | 1.00 | 99.99 | AAAA | C |
| ATOM | 2398 | O | LYS | A | 254 | 101.703 | 237.449 | 23.564 | 1.00 | 99.99 | AAAA | O |
| ATOM | 2399 | CB | LYS | A | 254 | 103.547 | 239.642 | 23.730 | 1.00 | 99.99 | AAAA | C |
| ATOM | 2400 | CG | LYS | A | 254 | 104.249 | 239.256 | 24.979 | 1.00 | 99.99 | AAAA | C |
| ATOM | 2401 | CD | LYS | A | 254 | 103.690 | 240.213 | 25.958 | 1.00 | 99.99 | AAAA | C |
| ATOM | 2402 | CE | LYS | A | 254 | 103.775 | 239.532 | 27.282 | 1.00 | 99.99 | AAAA | C |
| ATOM | 2403 | NZ | LYS | A | 254 | 103.577 | 240.626 | 28.213 | 1.00 | 99.99 | AAAA | N |
| ATOM | 2404 | H | LYS | A | 254 | 102.730 | 238.783 | 20.924 | 0.00 | 0.00 | AAAA | H |
| ATOM | 2405 | 1HZ | LYS | A | 254 | 103.884 | 240.333 | 29.158 | 0.00 | 0.00 | AAAA | H |
| ATOM | 2406 | 2HZ | LYS | A | 254 | 102.573 | 240.889 | 28.184 | 0.00 | 0.00 | AAAA | H |
| ATOM | 2407 | 3HZ | LYS | A | 254 | 104.157 | 241.420 | 27.867 | 0.00 | 0.00 | AAAA | H |
| ATOM | 2408 | N | TYR | A | 255 | 103.542 | 236.037 | 23.369 | 1.00 | 50.00 | AAAA | N |
| ATOM | 2409 | CA | TYR | A | 255 | 102.911 | 234.677 | 23.161 | 1.00 | 50.00 | AAAA | C |
| ATOM | 2410 | C | TYR | A | 255 | 103.033 | 234.206 | 24.631 | 1.00 | 50.00 | AAAA | C |
| ATOM | 2411 | O | TYR | A | 255 | 102.147 | 234.512 | 25.415 | 1.00 | 50.00 | AAAA | O |
| ATOM | 2412 | CB | TYR | A | 255 | 103.122 | 233.661 | 21.737 | 1.00 | 50.00 | AAAA | C |
| ATOM | 2413 | CG | TYR | A | 255 | 103.194 | 233.965 | 20.060 | 1.00 | 50.00 | AAAA | C |
| ATOM | 2414 | CD1 | TYR | A | 255 | 104.757 | 234.556 | 19.048 | 1.00 | 50.00 | AAAA | C |
| ATOM | 2415 | CD2 | TYR | A | 255 | 101.694 | 233.311 | 19.094 | 1.00 | 50.00 | AAAA | C |
| ATOM | 2416 | CE1 | TYR | A | 255 | 104.779 | 234.449 | 17.159 | 1.00 | 50.00 | AAAA | C |
| ATOM | 2417 | CE2 | TYR | A | 255 | 101.730 | 233.145 | 17.226 | 1.00 | 50.00 | AAAA | C |
| ATOM | 2418 | CZ | TYR | A | 255 | 103.264 | 233.735 | 16.257 | 1.00 | 50.00 | AAAA | C |
| ATOM | 2419 | OH | TYR | A | 255 | 103.039 | 233.982 | 14.944 | 1.00 | 50.00 | AAAA | O |
| ATOM | 2420 | H | TYR | A | 255 | 104.524 | 236.113 | 23.521 | 0.00 | 0.00 | AAAA | H |
| ATOM | 2421 | HH | TYR | A | 255 | 102.104 | 233.863 | 14.825 | 0.00 | 0.00 | AAAA | H |
| ATOM | 2422 | N | ASP | A | 256 | 104.191 | 233.653 | 25.060 | 1.00 | 50.00 | AAAA | N |
| ATOM | 2423 | CA | ASP | A | 256 | 104.397 | 232.204 | 25.146 | 1.00 | 50.00 | AAAA | C |
| ATOM | 2424 | C | ASP | A | 256 | 104.993 | 231.731 | 23.829 | 1.00 | 50.00 | AAAA | C |
| ATOM | 2425 | O | ASP | A | 256 | 104.831 | 230.612 | 23.357 | 1.00 | 50.00 | AAAA | O |
| ATOM | 2426 | CB | ASP | A | 256 | 103.110 | 231.471 | 25.594 | 1.00 | 50.00 | AAAA | C |
| ATOM | 2427 | CG | ASP | A | 256 | 102.657 | 231.968 | 26.973 | 1.00 | 50.00 | AAAA | C |
| ATOM | 2428 | OD1 | ASP | A | 256 | 103.493 | 232.056 | 27.874 | 1.00 | 50.00 | AAAA | O |
| ATOM | 2429 | OD2 | ASP | A | 256 | 101.473 | 232.257 | 27.139 | 1.00 | 50.00 | AAAA | O |
| ATOM | 2430 | H | ASP | A | 256 | 104.924 | 234.235 | 25.416 | 0.00 | 0.00 | AAAA | H |
| ATOM | 2431 | N | CYS | A | 257 | 105.664 | 232.765 | 23.256 | 1.00 | 50.00 | AAAA | N |
| ATOM | 2432 | CA | CYS | A | 257 | 106.120 | 232.984 | 21.887 | 1.00 | 50.00 | AAAA | C |
| ATOM | 2433 | C | CYS | A | 257 | 107.375 | 232.038 | 21.790 | 1.00 | 50.00 | AAAA | C |
| ATOM | 2434 | O | CYS | A | 257 | 108.222 | 232.081 | 22.672 | 1.00 | 50.00 | AAAA | O |
| ATOM | 2435 | CB | CYS | A | 257 | 106.097 | 234.649 | 21.782 | 1.00 | 50.00 | AAAA | C |
| ATOM | 2436 | SG | CYS | A | 257 | 104.675 | 235.974 | 21.125 | 1.00 | 50.00 | AAAA | S |
| ATOM | 2437 | H | CYS | A | 257 | 105.889 | 233.520 | 23.865 | 0.00 | 0.00 | AAAA | H |
| ATOM | 2438 | N | ARG | A | 258 | 107.437 | 231.107 | 20.782 | 1.00 | 50.00 | AAAA | N |
| ATOM | 2439 | CA | ARG | A | 258 | 108.609 | 230.200 | 20.722 | 1.00 | 50.00 | AAAA | C |
| ATOM | 2440 | C | ARG | A | 258 | 109.536 | 230.576 | 19.579 | 1.00 | 50.00 | AAAA | C |
| ATOM | 2441 | O | ARG | A | 258 | 109.094 | 230.780 | 18.455 | 1.00 | 50.00 | AAAA | O |
| ATOM | 2442 | CB | ARG | A | 258 | 108.235 | 228.689 | 20.648 | 1.00 | 50.00 | AAAA | C |
| ATOM | 2443 | CG | ARG | A | 258 | 107.646 | 228.156 | 19.321 | 1.00 | 50.00 | AAAA | C |
| ATOM | 2444 | CD | ARG | A | 258 | 107.165 | 226.692 | 19.325 | 1.00 | 50.00 | AAAA | C |
| ATOM | 2445 | NE | ARG | A | 258 | 106.562 | 226.304 | 18.041 | 1.00 | 50.00 | AAAA | N |
| ATOM | 2446 | CZ | ARG | A | 258 | 105.236 | 226.417 | 17.800 | 1.00 | 50.00 | AAAA | C |
| ATOM | 2447 | NH1 | ARG | A | 258 | 104.407 | 226.829 | 18.750 | 1.00 | 50.00 | AAAA | N |
| ATOM | 2448 | NH2 | ARG | A | 258 | 104.737 | 226.126 | 16.601 | 1.00 | 50.00 | AAAA | N |
| ATOM | 2449 | H | ARG | A | 258 | 106.760 | 230.992 | 20.058 | 0.00 | 0.00 | AAAA | H |
| ATOM | 2450 | HE | ARG | A | 258 | 107.146 | 225.971 | 17.303 | 0.00 | 0.00 | AAAA | H |
| ATOM | 2451 | 1HH1 | ARG | A | 258 | 103.428 | 226.912 | 18.569 | 0.00 | 0.00 | AAAA | H |
| ATOM | 2452 | 2HH1 | ARG | A | 258 | 104.774 | 227.081 | 19.646 | 0.00 | 0.00 | AAAA | H |
| ATOM | 2453 | 1HH2 | ARG | A | 258 | 103.756 | 226.222 | 16.430 | 0.00 | 0.00 | AAAA | H |
| ATOM | 2454 | 2HH2 | ARG | A | 258 | 105.338 | 225.817 | 15.862 | 0.00 | 0.00 | AAAA | H |
| ATOM | 2455 | N | ILE | A | 259 | 110.836 | 230.708 | 19.905 | 1.00 | 50.00 | AAAA | N |
| ATOM | 2456 | CA | ILE | A | 259 | 111.724 | 231.138 | 18.825 | 1.00 | 50.00 | AAAA | C |
| ATOM | 2457 | C | ILE | A | 259 | 112.878 | 230.174 | 18.598 | 1.00 | 50.00 | AAAA | C |
| ATOM | 2458 | O | ILE | A | 259 | 113.436 | 229.620 | 19.532 | 1.00 | 50.00 | AAAA | O |
| ATOM | 2459 | CB | ILE | A | 259 | 112.228 | 232.576 | 19.062 | 1.00 | 50.00 | AAAA | C |
| ATOM | 2460 | CG1 | ILE | A | 259 | 113.017 | 232.682 | 20.365 | 1.00 | 50.00 | AAAA | C |
| ATOM | 2461 | CG2 | ILE | A | 259 | 111.063 | 233.568 | 19.091 | 1.00 | 50.00 | AAAA | C |
| ATOM | 2462 | CD1 | ILE | A | 259 | 113.421 | 234.107 | 20.738 | 1.00 | 50.00 | AAAA | C |
| ATOM | 2463 | H | ILE | A | 259 | 111.169 | 230.520 | 20.830 | 0.00 | 0.00 | AAAA | H |
| ATOM | 2464 | N | MET | A | 260 | 113.216 | 229.999 | 17.311 | 1.00 | 50.00 | AAAA | N |
| ATOM | 2465 | CA | MET | A | 260 | 114.362 | 229.144 | 17.018 | 1.00 | 50.00 | AAAA | C |
| ATOM | 2466 | C | MET | A | 260 | 115.481 | 229.931 | 16.372 | 1.00 | 50.00 | AAAA | C |
| ATOM | 2467 | O | MET | A | 260 | 115.266 | 230.684 | 15.431 | 1.00 | 50.00 | AAAA | O |
| ATOM | 2468 | CB | MET | A | 260 | 113.955 | 227.969 | 16.116 | 1.00 | 50.00 | AAAA | C |
| ATOM | 2469 | CG | MET | A | 260 | 115.114 | 226.998 | 15.851 | 1.00 | 50.00 | AAAA | C |
| ATOM | 2470 | SD | MET | A | 260 | 114.747 | 225.707 | 14.659 | 1.00 | 50.00 | AAAA | S |
| ATOM | 2471 | CE | MET | A | 260 | 116.423 | 225.131 | 14.364 | 1.00 | 50.00 | AAAA | C |
| ATOM | 2472 | H | MET | A | 260 | 112.732 | 230.459 | 16.571 | 0.00 | 0.00 | AAAA | H |
| ATOM | 2473 | N | THR | A | 261 | 116.700 | 229.710 | 16.896 | 1.00 | 50.00 | AAAA | N |
| ATOM | 2474 | CA | THR | A | 261 | 117.851 | 230.309 | 16.220 | 1.00 | 50.00 | AAAA | C |
| ATOM | 2475 | C | THR | A | 261 | 118.017 | 229.898 | 14.760 | 1.00 | 50.00 | AAAA | C |
| ATOM | 2476 | O | THR | A | 261 | 117.937 | 228.736 | 14.380 | 1.00 | 50.00 | AAAA | O |
| ATOM | 2477 | CB | THR | A | 261 | 119.135 | 230.060 | 17.019 | 1.00 | 50.00 | AAAA | C |
| ATOM | 2478 | CG2 | THR | A | 261 | 119.046 | 230.671 | 18.422 | 1.00 | 50.00 | AAAA | C |
| ATOM | 2479 | OG1 | THR | A | 261 | 119.424 | 228.658 | 17.113 | 1.00 | 50.00 | AAAA | O |
| ATOM | 2480 | H | THR | A | 261 | 116.843 | 229.070 | 17.647 | 0.00 | 0.00 | AAAA | H |
| ATOM | 2481 | HG1 | THR | A | 261 | 120.302 | 228.586 | 17.462 | 0.00 | 0.00 | AAAA | H |
| ATOM | 2482 | N | SER | A | 262 | 118.219 | 230.943 | 13.944 | 1.00 | 50.00 | AAAA | N |
| ATOM | 2483 | CA | SER | A | 262 | 118.300 | 230.723 | 12.508 | 1.00 | 50.00 | AAAA | C |
| ATOM | 2484 | C | SER | A | 262 | 119.485 | 231.449 | 11.900 | 1.00 | 50.00 | AAAA | C |
| ATOM | 2485 | O | SER | A | 262 | 119.990 | 232.432 | 12.424 | 1.00 | 50.00 | AAAA | O |
| ATOM | 2486 | CB | SER | A | 262 | 116.975 | 231.152 | 11.859 | 1.00 | 50.00 | AAAA | C |
| ATOM | 2487 | OG | SER | A | 262 | 116.970 | 230.898 | 10.449 | 1.00 | 50.00 | AAAA | O |
| ATOM | 2488 | H | SER | A | 262 | 118.301 | 231.868 | 14.303 | 0.00 | 0.00 | AAAA | H |
| ATOM | 2489 | HG | SER | A | 262 | 116.241 | 231.369 | 10.070 | 0.00 | 0.00 | AAAA | H |
| ATOM | 2490 | N | LYS | A | 263 | 119.908 | 230.909 | 10.749 | 1.00 | 50.00 | AAAA | N |
| ATOM | 2491 | CA | LYS | A | 263 | 120.943 | 231.592 | 9.979 | 1.00 | 50.00 | AAAA | C |
| ATOM | 2492 | C | LYS | A | 263 | 120.355 | 232.623 | 9.026 | 1.00 | 50.00 | AAAA | C |
| ATOM | 2493 | O | LYS | A | 263 | 119.860 | 232.293 | 7.955 | 1.00 | 50.00 | AAAA | O |
| ATOM | 2494 | CB | LYS | A | 263 | 121.786 | 230.551 | 9.236 | 1.00 | 50.00 | AAAA | C |
| ATOM | 2495 | CG | LYS | A | 263 | 123.034 | 231.104 | 8.541 | 1.00 | 50.00 | AAAA | C |
| ATOM | 2496 | CD | LYS | A | 263 | 124.056 | 231.684 | 9.521 | 1.00 | 50.00 | AAAA | C |
| ATOM | 2497 | CE | LYS | A | 263 | 125.306 | 232.221 | 8.820 | 1.00 | 50.00 | AAAA | C |
| ATOM | 2498 | NZ | LYS | A | 263 | 124.947 | 233.352 | 7.953 | 1.00 | 50.00 | AAAA | N |
| ATOM | 2499 | H | LYS | A | 263 | 119.407 | 230.137 | 10.360 | 0.00 | 0.00 | AAAA | H |
| ATOM | 2500 | 1HZ | LYS | A | 263 | 125.804 | 233.697 | 7.476 | 0.00 | 0.00 | AAAA | H |
| ATOM | 2501 | 2HZ | LYS | A | 263 | 124.537 | 234.112 | 8.531 | 0.00 | 0.00 | AAAA | H |
| ATOM | 2502 | 3HZ | LYS | A | 263 | 124.258 | 233.037 | 7.241 | 0.00 | 0.00 | AAAA | H |
| ATOM | 2503 | N | THR | A | 264 | 120.436 | 233.893 | 9.469 | 1.00 | 50.00 | AAAA | N |
| ATOM | 2504 | CA | THR | A | 264 | 119.872 | 234.960 | 8.640 | 1.00 | 50.00 | AAAA | C |
| ATOM | 2505 | C | THR | A | 264 | 120.427 | 235.123 | 7.234 | 1.00 | 50.00 | AAAA | C |
| ATOM | 2506 | O | THR | A | 264 | 121.531 | 234.706 | 6.908 | 1.00 | 50.00 | AAAA | O |
| ATOM | 2507 | CB | THR | A | 264 | 119.867 | 236.308 | 9.383 | 1.00 | 50.00 | AAAA | C |
| ATOM | 2508 | CG2 | THR | A | 264 | 121.254 | 236.933 | 9.561 | 1.00 | 50.00 | AAAA | C |
| ATOM | 2509 | OG1 | THR | A | 264 | 118.979 | 237.208 | 8.715 | 1.00 | 50.00 | AAAA | O |
| ATOM | 2510 | H | THR | A | 264 | 120.760 | 234.061 | 10.399 | 0.00 | 0.00 | AAAA | H |
| ATOM | 2511 | HG1 | THR | A | 264 | 118.906 | 237.997 | 9.237 | 0.00 | 0.00 | AAAA | H |
| ATOM | 2512 | N | ASP | A | 265 | 119.581 | 235.783 | 6.426 | 1.00 | 50.00 | AAAA | N |
| ATOM | 2513 | CA | ASP | A | 265 | 119.974 | 236.176 | 5.080 | 1.00 | 50.00 | AAAA | C |
| ATOM | 2514 | C | ASP | A | 265 | 119.230 | 237.454 | 4.724 | 1.00 | 50.00 | AAAA | C |
| ATOM | 2515 | O | ASP | A | 265 | 118.274 | 237.822 | 5.394 | 1.00 | 50.00 | AAAA | O |
| ATOM | 2516 | CB | ASP | A | 265 | 119.669 | 235.031 | 4.105 | 1.00 | 50.00 | AAAA | C |
| ATOM | 2517 | CG | ASP | A | 265 | 120.279 | 235.311 | 2.749 | 1.00 | 50.00 | AAAA | C |
| ATOM | 2518 | OD1 | ASP | A | 265 | 121.495 | 235.494 | 2.670 | 1.00 | 50.00 | AAAA | O |
| ATOM | 2519 | OD2 | ASP | A | 265 | 119.532 | 235.377 | 1.775 | 1.00 | 50.00 | AAAA | O |
| ATOM | 2520 | H | ASP | A | 265 | 118.680 | 236.039 | 6.778 | 0.00 | 0.00 | AAAA | H |
| ATOM | 2521 | N | GLN | A | 266 | 119.690 | 238.108 | 3.635 | 1.00 | 50.00 | AAAA | N |
| ATOM | 2522 | CA | GLN | A | 266 | 119.015 | 239.322 | 3.159 | 1.00 | 50.00 | AAAA | C |
| ATOM | 2523 | C | GLN | A | 266 | 117.511 | 239.156 | 2.949 | 1.00 | 50.00 | AAAA | C |
| ATOM | 2524 | O | GLN | A | 266 | 116.705 | 239.997 | 3.325 | 1.00 | 50.00 | AAAA | O |
| ATOM | 2525 | CB | GLN | A | 266 | 119.694 | 239.812 | 1.876 | 1.00 | 50.00 | AAAA | C |
| ATOM | 2526 | CG | GLN | A | 266 | 119.183 | 241.170 | 1.384 | 1.00 | 50.00 | AAAA | C |
| ATOM | 2527 | CD | GLN | A | 266 | 119.842 | 241.507 | 0.064 | 1.00 | 50.00 | AAAA | C |
| ATOM | 2528 | NE2 | GLN | A | 266 | 120.472 | 242.689 | 0.078 | 1.00 | 50.00 | AAAA | N |
| ATOM | 2529 | OE1 | GLN | A | 266 | 119.785 | 240.757 | −0.902 | 1.00 | 50.00 | AAAA | O |
| ATOM | 2530 | H | GLN | A | 266 | 120.509 | 237.779 | 3.164 | 0.00 | 0.00 | AAAA | H |
| ATOM | 2531 | 1HE2 | GLN | A | 266 | 120.923 | 243.033 | −0.746 | 0.00 | 0.00 | AAAA | H |
| ATOM | 2532 | 2HE2 | GLN | A | 266 | 120.511 | 243.252 | 0.902 | 0.00 | 0.00 | AAAA | H |
| ATOM | 2533 | N | SER | A | 267 | 117.171 | 237.993 | 2.361 | 1.00 | 50.00 | AAAA | N |
| ATOM | 2534 | CA | SER | A | 267 | 115.768 | 237.684 | 2.070 | 1.00 | 50.00 | AAAA | C |
| ATOM | 2535 | C | SER | A | 267 | 114.834 | 237.514 | 3.262 | 1.00 | 50.00 | AAAA | C |
| ATOM | 2536 | O | SER | A | 267 | 113.619 | 237.535 | 3.119 | 1.00 | 50.00 | AAAA | O |
| ATOM | 2537 | CB | SER | A | 267 | 115.682 | 236.437 | 1.187 | 1.00 | 50.00 | AAAA | C |
| ATOM | 2538 | OG | SER | A | 267 | 116.476 | 236.610 | 0.011 | 1.00 | 50.00 | AAAA | O |
| ATOM | 2539 | H | SER | A | 267 | 117.884 | 237.344 | 2.095 | 0.00 | 0.00 | AAAA | H |
| ATOM | 2540 | HG | SER | A | 267 | 116.307 | 235.858 | −0.544 | 0.00 | 0.00 | AAAA | H |
| ATOM | 2541 | N | SER | A | 268 | 115.459 | 237.332 | 4.438 | 1.00 | 50.00 | AAAA | N |
| ATOM | 2542 | CA | SER | A | 268 | 114.650 | 237.067 | 5.622 | 1.00 | 50.00 | AAAA | C |
| ATOM | 2543 | C | SER | A | 268 | 114.847 | 238.069 | 6.747 | 1.00 | 50.00 | AAAA | C |
| ATOM | 2544 | O | SER | A | 268 | 114.430 | 237.847 | 7.873 | 1.00 | 50.00 | AAAA | O |
| ATOM | 2545 | CB | SER | A | 268 | 114.903 | 235.635 | 6.119 | 1.00 | 50.00 | AAAA | C |
| ATOM | 2546 | OG | SER | A | 268 | 114.069 | 235.323 | 7.246 | 1.00 | 50.00 | AAAA | O |
| ATOM | 2547 | H | SER | A | 268 | 116.452 | 237.363 | 4.526 | 0.00 | 0.00 | AAAA | H |
| ATOM | 2548 | HG | SER | A | 268 | 114.123 | 234.387 | 7.386 | 0.00 | 0.00 | AAAA | H |
| ATOM | 2549 | N | SER | A | 269 | 115.509 | 239.186 | 6.426 | 1.00 | 50.00 | AAAA | N |
| ATOM | 2550 | CA | SER | A | 269 | 115.710 | 240.103 | 7.540 | 1.00 | 50.00 | AAAA | C |
| ATOM | 2551 | C | SER | A | 269 | 114.801 | 241.305 | 7.544 | 1.00 | 50.00 | AAAA | C |
| ATOM | 2552 | O | SER | A | 269 | 114.950 | 242.187 | 8.374 | 1.00 | 50.00 | AAAA | O |
| ATOM | 2553 | CB | SER | A | 269 | 117.163 | 240.544 | 7.591 | 1.00 | 50.00 | AAAA | C |
| ATOM | 2554 | OG | SER | A | 269 | 117.985 | 239.385 | 7.697 | 1.00 | 50.00 | AAAA | O |
| ATOM | 2555 | H | SER | A | 269 | 115.843 | 239.380 | 5.506 | 0.00 | 0.00 | AAAA | H |
| ATOM | 2556 | HG | SER | A | 269 | 118.879 | 239.670 | 7.806 | 0.00 | 0.00 | AAAA | H |
| ATOM | 2557 | N | VAL | A | 270 | 113.896 | 241.324 | 6.548 | 1.00 | 50.00 | AAAA | N |
| ATOM | 2558 | CA | VAL | A | 270 | 113.153 | 242.555 | 6.284 | 1.00 | 50.00 | AAAA | C |
| ATOM | 2559 | C | VAL | A | 270 | 111.947 | 242.286 | 5.402 | 1.00 | 50.00 | AAAA | C |
| ATOM | 2560 | O | VAL | A | 270 | 112.018 | 241.476 | 4.486 | 1.00 | 50.00 | AAAA | O |
| ATOM | 2561 | CB | VAL | A | 270 | 114.107 | 243.577 | 5.622 | 1.00 | 50.00 | AAAA | C |
| ATOM | 2562 | CG1 | VAL | A | 270 | 114.782 | 243.050 | 4.355 | 1.00 | 50.00 | AAAA | C |
| ATOM | 2563 | CG2 | VAL | A | 270 | 113.430 | 244.904 | 5.334 | 1.00 | 50.00 | AAAA | C |
| ATOM | 2564 | H | VAL | A | 270 | 113.751 | 240.523 | 5.968 | 0.00 | 0.00 | AAAA | H |
| ATOM | 2565 | N | ILE | A | 271 | 110.845 | 243.015 | 5.688 | 1.00 | 50.00 | AAAA | N |
| ATOM | 2566 | CA | ILE | A | 271 | 109.714 | 242.989 | 4.754 | 1.00 | 50.00 | AAAA | C |
| ATOM | 2567 | C | ILE | A | 271 | 109.124 | 244.385 | 4.566 | 1.00 | 50.00 | AAAA | C |
| ATOM | 2568 | O | ILE | A | 271 | 109.344 | 245.295 | 5.362 | 1.00 | 50.00 | AAAA | O |
| ATOM | 2569 | CB | ILE | A | 271 | 108.619 | 241.999 | 5.195 | 1.00 | 50.00 | AAAA | C |
| ATOM | 2570 | CG1 | ILE | A | 271 | 108.035 | 242.467 | 6.520 | 1.00 | 50.00 | AAAA | C |
| ATOM | 2571 | CG2 | ILE | A | 271 | 109.145 | 240.558 | 5.321 | 1.00 | 50.00 | AAAA | C |
| ATOM | 2572 | CD1 | ILE | A | 271 | 107.224 | 241.392 | 7.211 | 1.00 | 50.00 | AAAA | C |
| ATOM | 2573 | H | ILE | A | 271 | 110.834 | 243.639 | 6.474 | 0.00 | 0.00 | AAAA | H |
| ATOM | 2574 | N | THR | A | 272 | 108.359 | 244.499 | 3.463 | 1.00 | 50.00 | AAAA | N |
| ATOM | 2575 | CA | THR | A | 272 | 107.669 | 245.760 | 3.199 | 1.00 | 50.00 | AAAA | C |
| ATOM | 2576 | C | THR | A | 272 | 106.184 | 245.683 | 3.545 | 1.00 | 50.00 | AAAA | C |
| ATOM | 2577 | O | THR | A | 272 | 105.415 | 244.956 | 2.933 | 1.00 | 50.00 | AAAA | O |
| ATOM | 2578 | CB | THR | A | 272 | 107.880 | 246.195 | 1.732 | 1.00 | 50.00 | AAAA | C |
| ATOM | 2579 | CG2 | THR | A | 272 | 109.356 | 246.189 | 1.327 | 1.00 | 50.00 | AAAA | C |
| ATOM | 2580 | OG1 | THR | A | 272 | 107.177 | 245.342 | 0.826 | 1.00 | 50.00 | AAAA | O |
| ATOM | 2581 | H | THR | A | 272 | 108.189 | 243.717 | 2.863 | 0.00 | 0.00 | AAAA | H |
| ATOM | 2582 | HG1 | THR | A | 272 | 107.335 | 245.686 | −0.046 | 0.00 | 0.00 | AAAA | H |
| ATOM | 2583 | N | SER | A | 273 | 105.815 | 246.468 | 4.565 | 1.00 | 50.00 | AAAA | N |
| ATOM | 2584 | CA | SER | A | 273 | 104.428 | 246.472 | 5.014 | 1.00 | 50.00 | AAAA | C |
| ATOM | 2585 | C | SER | A | 273 | 103.773 | 247.823 | 4.797 | 1.00 | 50.00 | AAAA | C |
| ATOM | 2586 | O | SER | A | 273 | 103.859 | 248.725 | 5.624 | 1.00 | 50.00 | AAAA | O |
| ATOM | 2587 | CB | SER | A | 273 | 104.360 | 246.042 | 6.486 | 1.00 | 50.00 | AAAA | C |
| ATOM | 2588 | OG | SER | A | 273 | 103.005 | 245.823 | 6.902 | 1.00 | 50.00 | AAAA | O |
| ATOM | 2589 | H | SER | A | 273 | 106.481 | 247.048 | 5.023 | 0.00 | 0.00 | AAAA | H |
| ATOM | 2590 | HG | SER | A | 273 | 103.010 | 245.886 | 7.849 | 0.00 | 0.00 | AAAA | H |
| ATOM | 2591 | N | LEU | A | 274 | 103.108 | 247.900 | 3.624 | 1.00 | 50.00 | AAAA | N |
| ATOM | 2592 | CA | LEU | A | 274 | 102.339 | 249.088 | 3.242 | 1.00 | 50.00 | AAAA | C |
| ATOM | 2593 | C | LEU | A | 274 | 103.140 | 250.377 | 3.175 | 1.00 | 50.00 | AAAA | C |
| ATOM | 2594 | O | LEU | A | 274 | 102.665 | 251.441 | 3.539 | 1.00 | 50.00 | AAAA | O |
| ATOM | 2595 | CB | LEU | A | 274 | 101.082 | 249.287 | 4.109 | 1.00 | 50.00 | AAAA | C |
| ATOM | 2596 | CG | LEU | A | 274 | 99.852 | 248.438 | 3.758 | 1.00 | 50.00 | AAAA | C |
| ATOM | 2597 | CD1 | LEU | A | 274 | 100.056 | 246.930 | 3.925 | 1.00 | 50.00 | AAAA | C |
| ATOM | 2598 | CD2 | LEU | A | 274 | 98.635 | 248.916 | 4.551 | 1.00 | 50.00 | AAAA | C |
| ATOM | 2599 | H | LEU | A | 274 | 103.077 | 247.080 | 3.056 | 0.00 | 0.00 | AAAA | H |
| ATOM | 2600 | N | GLY | A | 275 | 104.379 | 250.229 | 2.672 | 1.00 | 50.00 | AAAA | N |
| ATOM | 2601 | CA | GLY | A | 275 | 105.200 | 251.424 | 2.494 | 1.00 | 50.00 | AAAA | C |
| ATOM | 2602 | C | GLY | A | 275 | 106.249 | 251.620 | 3.566 | 1.00 | 50.00 | AAAA | C |
| ATOM | 2603 | O | GLY | A | 275 | 107.239 | 252.320 | 3.381 | 1.00 | 50.00 | AAAA | O |
| ATOM | 2604 | H | GLY | A | 275 | 104.755 | 249.319 | 2.501 | 0.00 | 0.00 | AAAA | H |
| ATOM | 2605 | N | ALA | A | 276 | 105.996 | 250.966 | 4.708 | 1.00 | 50.00 | AAAA | N |
| ATOM | 2606 | CA | ALA | A | 276 | 107.041 | 251.072 | 5.708 | 1.00 | 50.00 | AAAA | C |
| ATOM | 2607 | C | ALA | A | 276 | 107.668 | 249.734 | 6.001 | 1.00 | 50.00 | AAAA | C |
| ATOM | 2608 | O | ALA | A | 276 | 107.159 | 248.687 | 5.637 | 1.00 | 50.00 | AAAA | O |
| ATOM | 2609 | CB | ALA | A | 276 | 106.490 | 251.708 | 6.960 | 1.00 | 50.00 | AAAA | C |
| ATOM | 2610 | H | ALA | A | 276 | 105.185 | 250.398 | 4.869 | 0.00 | 0.00 | AAAA | H |
| ATOM | 2611 | N | ILE | A | 277 | 108.845 | 249.809 | 6.623 | 1.00 | 50.00 | AAAA | N |
| ATOM | 2612 | CA | ILE | A | 277 | 109.640 | 248.588 | 6.598 | 1.00 | 50.00 | AAAA | C |
| ATOM | 2613 | C | ILE | A | 277 | 109.804 | 248.008 | 8.005 | 1.00 | 50.00 | AAAA | C |
| ATOM | 2614 | O | ILE | A | 277 | 109.803 | 248.736 | 8.988 | 1.00 | 50.00 | AAAA | O |
| ATOM | 2615 | CB | ILE | A | 277 | 110.964 | 248.986 | 5.923 | 1.00 | 50.00 | AAAA | C |
| ATOM | 2616 | CG1 | ILE | A | 277 | 110.830 | 249.664 | 4.557 | 1.00 | 50.00 | AAAA | C |
| ATOM | 2617 | CG2 | ILE | A | 277 | 111.973 | 247.859 | 5.840 | 1.00 | 50.00 | AAAA | C |
| ATOM | 2618 | CD1 | ILE | A | 277 | 110.294 | 248.748 | 3.470 | 1.00 | 50.00 | AAAA | C |
| ATOM | 2619 | H | ILE | A | 277 | 109.219 | 250.671 | 6.965 | 0.00 | 0.00 | AAAA | H |
| ATOM | 2620 | N | VAL | A | 278 | 109.940 | 246.670 | 8.074 | 1.00 | 50.00 | AAAA | N |
| ATOM | 2621 | CA | VAL | A | 278 | 110.280 | 246.087 | 9.374 | 1.00 | 50.00 | AAAA | C |
| ATOM | 2622 | C | VAL | A | 278 | 111.401 | 245.073 | 9.257 | 1.00 | 50.00 | AAAA | C |
| ATOM | 2623 | O | VAL | A | 278 | 111.285 | 243.999 | 8.676 | 1.00 | 50.00 | AAAA | O |
| ATOM | 2624 | CB | VAL | A | 278 | 109.042 | 245.534 | 10.112 | 1.00 | 50.00 | AAAA | C |
| ATOM | 2625 | CG1 | VAL | A | 278 | 108.141 | 244.709 | 9.201 | 1.00 | 50.00 | AAAA | C |
| ATOM | 2626 | CG2 | VAL | A | 278 | 109.391 | 244.794 | 11.408 | 1.00 | 50.00 | AAAA | C |
| ATOM | 2627 | H | VAL | A | 278 | 109.842 | 246.087 | 7.267 | 0.00 | 0.00 | AAAA | H |
| ATOM | 2628 | N | SER | A | 279 | 112.517 | 245.517 | 9.847 | 1.00 | 50.00 | AAAA | N |
| ATOM | 2629 | CA | SER | A | 279 | 113.728 | 244.737 | 9.736 | 1.00 | 50.00 | AAAA | C |
| ATOM | 2630 | C | SER | A | 279 | 114.457 | 244.579 | 11.062 | 1.00 | 50.00 | AAAA | C |
| ATOM | 2631 | O | SER | A | 279 | 114.152 | 245.153 | 12.094 | 1.00 | 50.00 | AAAA | O |
| ATOM | 2632 | CB | SER | A | 279 | 114.607 | 245.400 | 8.661 | 1.00 | 50.00 | AAAA | C |
| ATOM | 2633 | OG | SER | A | 279 | 115.746 | 244.605 | 8.324 | 1.00 | 50.00 | AAAA | O |
| ATOM | 2634 | H | SER | A | 279 | 112.545 | 246.389 | 10.333 | 0.00 | 0.00 | AAAA | H |
| ATOM | 2635 | HG | SER | A | 279 | 116.379 | 245.168 | 7.902 | 0.00 | 0.00 | AAAA | H |
| ATOM | 2636 | N | CYS | A | 280 | 115.490 | 243.763 | 10.908 | 1.00 | 50.00 | AAAA | N |
| ATOM | 2637 | CA | CYS | A | 280 | 116.682 | 243.483 | 11.689 | 1.00 | 50.00 | AAAA | C |
| ATOM | 2638 | C | CYS | A | 280 | 117.635 | 244.576 | 12.139 | 1.00 | 50.00 | AAAA | C |
| ATOM | 2639 | O | CYS | A | 280 | 118.769 | 244.467 | 11.688 | 1.00 | 50.00 | AAAA | O |
| ATOM | 2640 | CB | CYS | A | 280 | 117.546 | 242.740 | 10.693 | 1.00 | 50.00 | AAAA | C |
| ATOM | 2641 | SG | CYS | A | 280 | 118.316 | 243.843 | 9.360 | 1.00 | 50.00 | AAAA | S |
| ATOM | 2642 | H | CYS | A | 280 | 115.442 | 243.227 | 10.063 | 0.00 | 0.00 | AAAA | H |
| ATOM | 2643 | N | TYR | A | 281 | 117.338 | 245.592 | 12.967 | 1.00 | 50.00 | AAAA | N |
| ATOM | 2644 | CA | TYR | A | 281 | 118.492 | 246.503 | 12.826 | 1.00 | 50.00 | AAAA | C |
| ATOM | 2645 | C | TYR | A | 281 | 119.846 | 246.060 | 13.439 | 1.00 | 50.00 | AAAA | C |
| ATOM | 2646 | O | TYR | A | 281 | 120.866 | 246.712 | 13.268 | 1.00 | 50.00 | AAAA | O |
| ATOM | 2647 | CB | TYR | A | 281 | 118.097 | 247.977 | 12.980 | 1.00 | 50.00 | AAAA | C |
| ATOM | 2648 | CG | TYR | A | 281 | 117.123 | 248.419 | 11.896 | 1.00 | 50.00 | AAAA | C |
| ATOM | 2649 | CD1 | TYR | A | 281 | 116.123 | 247.566 | 11.375 | 1.00 | 50.00 | AAAA | C |
| ATOM | 2650 | CD2 | TYR | A | 281 | 117.256 | 249.729 | 11.417 | 1.00 | 50.00 | AAAA | C |
| ATOM | 2651 | CE1 | TYR | A | 281 | 115.319 | 247.999 | 10.316 | 1.00 | 50.00 | AAAA | C |
| ATOM | 2652 | CE2 | TYR | A | 281 | 116.455 | 250.171 | 10.357 | 1.00 | 50.00 | AAAA | C |
| ATOM | 2653 | CZ | TYR | A | 281 | 115.548 | 249.272 | 9.779 | 1.00 | 50.00 | AAAA | C |
| ATOM | 2654 | OH | TYR | A | 281 | 114.902 | 249.643 | 8.629 | 1.00 | 50.00 | AAAA | O |
| ATOM | 2655 | H | TYR | A | 281 | 116.487 | 245.807 | 13.455 | 0.00 | 0.00 | AAAA | H |
| ATOM | 2656 | HH | TYR | A | 281 | 114.877 | 250.589 | 8.587 | 0.00 | 0.00 | AAAA | H |
| ATOM | 2657 | N | GLY | A | 282 | 119.805 | 244.852 | 14.072 | 1.00 | 50.00 | AAAA | N |
| ATOM | 2658 | CA | GLY | A | 282 | 121.016 | 244.220 | 14.595 | 1.00 | 50.00 | AAAA | C |
| ATOM | 2659 | C | GLY | A | 282 | 121.719 | 243.228 | 13.678 | 1.00 | 50.00 | AAAA | C |
| ATOM | 2660 | O | GLY | A | 282 | 122.938 | 243.227 | 13.572 | 1.00 | 50.00 | AAAA | O |
| ATOM | 2661 | H | GLY | A | 282 | 118.943 | 244.345 | 14.081 | 0.00 | 0.00 | AAAA | H |
| ATOM | 2662 | N | LYS | A | 283 | 120.919 | 242.352 | 13.036 | 1.00 | 50.00 | AAAA | N |
| ATOM | 2663 | CA | LYS | A | 283 | 121.584 | 241.357 | 12.186 | 1.00 | 50.00 | AAAA | C |
| ATOM | 2664 | C | LYS | A | 283 | 121.929 | 241.787 | 10.781 | 1.00 | 50.00 | AAAA | C |
| ATOM | 2665 | O | LYS | A | 283 | 122.815 | 241.221 | 10.149 | 1.00 | 50.00 | AAAA | O |
| ATOM | 2666 | CB | LYS | A | 283 | 120.799 | 240.062 | 12.140 | 1.00 | 50.00 | AAAA | C |
| ATOM | 2667 | CG | LYS | A | 283 | 120.729 | 239.492 | 13.544 | 1.00 | 50.00 | AAAA | C |
| ATOM | 2668 | CD | LYS | A | 283 | 122.025 | 238.908 | 14.096 | 1.00 | 50.00 | AAAA | C |
| ATOM | 2669 | CE | LYS | A | 283 | 121.819 | 238.537 | 15.564 | 1.00 | 50.00 | AAAA | C |
| ATOM | 2670 | NZ | LYS | A | 283 | 122.783 | 237.507 | 15.970 | 1.00 | 50.00 | AAAA | N |
| ATOM | 2671 | H | LYS | A | 283 | 119.924 | 242.414 | 13.101 | 0.00 | 0.00 | AAAA | H |
| ATOM | 2672 | 1HZ | LYS | A | 283 | 122.521 | 237.156 | 16.912 | 0.00 | 0.00 | AAAA | H |
| ATOM | 2673 | 2HZ | LYS | A | 283 | 123.737 | 237.914 | 15.983 | 0.00 | 0.00 | AAAA | H |
| ATOM | 2674 | 3HZ | LYS | A | 283 | 122.738 | 236.723 | 15.286 | 0.00 | 0.00 | AAAA | H |
| ATOM | 2675 | N | THR | A | 284 | 121.210 | 242.821 | 10.321 | 1.00 | 50.00 | AAAA | N |
| ATOM | 2676 | CA | THR | A | 284 | 121.761 | 243.393 | 9.104 | 1.00 | 50.00 | AAAA | C |
| ATOM | 2677 | C | THR | A | 284 | 122.064 | 244.859 | 9.251 | 1.00 | 50.00 | AAAA | C |
| ATOM | 2678 | O | THR | A | 284 | 121.618 | 245.532 | 10.174 | 1.00 | 50.00 | AAAA | O |
| ATOM | 2679 | CB | THR | A | 284 | 121.025 | 243.055 | 7.771 | 1.00 | 50.00 | AAAA | C |
| ATOM | 2680 | CG2 | THR | A | 284 | 120.595 | 241.596 | 7.675 | 1.00 | 50.00 | AAAA | C |
| ATOM | 2681 | OG1 | THR | A | 284 | 119.950 | 243.937 | 7.432 | 1.00 | 50.00 | AAAA | O |
| ATOM | 2682 | H | THR | A | 284 | 120.526 | 243.288 | 10.881 | 0.00 | 0.00 | AAAA | H |
| ATOM | 2683 | HG1 | THR | A | 284 | 119.138 | 243.464 | 7.591 | 0.00 | 0.00 | AAAA | H |
| ATOM | 2684 | N | LYS | A | 285 | 122.897 | 245.297 | 8.290 | 1.00 | 50.00 | AAAA | N |
| ATOM | 2685 | CA | LYS | A | 285 | 123.304 | 246.691 | 8.290 | 1.00 | 50.00 | AAAA | C |
| ATOM | 2686 | C | LYS | A | 285 | 122.221 | 247.485 | 7.624 | 1.00 | 50.00 | AAAA | C |
| ATOM | 2687 | O | LYS | A | 285 | 122.117 | 247.660 | 6.414 | 1.00 | 50.00 | AAAA | O |
| ATOM | 2688 | CB | LYS | A | 285 | 124.662 | 246.864 | 7.616 | 1.00 | 50.00 | AAAA | C |
| ATOM | 2689 | CG | LYS | A | 285 | 125.234 | 248.278 | 7.763 | 1.00 | 50.00 | AAAA | C |
| ATOM | 2690 | CD | LYS | A | 285 | 125.425 | 248.744 | 9.210 | 1.00 | 50.00 | AAAA | C |
| ATOM | 2691 | CE | LYS | A | 285 | 125.976 | 250.173 | 9.346 | 1.00 | 50.00 | AAAA | C |
| ATOM | 2692 | NZ | LYS | A | 285 | 124.952 | 251.166 | 8.988 | 1.00 | 50.00 | AAAA | N |
| ATOM | 2693 | H | LYS | A | 285 | 123.077 | 244.722 | 7.495 | 0.00 | 0.00 | AAAA | H |
| ATOM | 2694 | 1HZ | LYS | A | 285 | 125.365 | 252.118 | 8.917 | 0.00 | 0.00 | AAAA | H |
| ATOM | 2695 | 2HZ | LYS | A | 285 | 124.178 | 251.176 | 9.681 | 0.00 | 0.00 | AAAA | H |
| ATOM | 2696 | 3HZ | LYS | A | 285 | 124.549 | 250.915 | 8.068 | 0.00 | 0.00 | AAAA | H |
| ATOM | 2697 | N | CYS | A | 286 | 121.351 | 247.866 | 8.544 | 1.00 | 50.00 | AAAA | N |
| ATOM | 2698 | CA | CYS | A | 286 | 120.065 | 248.356 | 8.131 | 1.00 | 50.00 | AAAA | C |
| ATOM | 2699 | C | CYS | A | 286 | 120.337 | 249.936 | 8.042 | 1.00 | 50.00 | AAAA | C |
| ATOM | 2700 | O | CYS | A | 286 | 120.017 | 250.691 | 8.952 | 1.00 | 50.00 | AAAA | O |
| ATOM | 2701 | CB | CYS | A | 286 | 119.143 | 247.562 | 9.206 | 1.00 | 50.00 | AAAA | C |
| ATOM | 2702 | SG | CYS | A | 286 | 118.049 | 245.985 | 8.979 | 1.00 | 50.00 | AAAA | S |
| ATOM | 2703 | H | CYS | A | 286 | 121.588 | 247.868 | 9.517 | 0.00 | 0.00 | AAAA | H |
| ATOM | 2704 | N | THR | A | 287 | 121.043 | 250.393 | 6.908 | 1.00 | 50.00 | AAAA | N |
| ATOM | 2705 | CA | THR | A | 287 | 121.681 | 251.744 | 6.655 | 1.00 | 50.00 | AAAA | C |
| ATOM | 2706 | C | THR | A | 287 | 120.831 | 252.946 | 6.196 | 1.00 | 50.00 | AAAA | C |
| ATOM | 2707 | O | THR | A | 287 | 120.418 | 253.029 | 5.048 | 1.00 | 50.00 | AAAA | O |
| ATOM | 2708 | CB | THR | A | 287 | 122.887 | 251.778 | 5.634 | 1.00 | 50.00 | AAAA | C |
| ATOM | 2709 | CG2 | THR | A | 287 | 124.115 | 250.954 | 5.934 | 1.00 | 50.00 | AAAA | C |
| ATOM | 2710 | OG1 | THR | A | 287 | 122.506 | 251.455 | 4.298 | 1.00 | 50.00 | AAAA | O |
| ATOM | 2711 | H | THR | A | 287 | 121.174 | 249.733 | 6.168 | 0.00 | 0.00 | AAAA | H |
| ATOM | 2712 | HG1 | THR | A | 287 | 123.283 | 251.547 | 3.758 | 0.00 | 0.00 | AAAA | H |
| ATOM | 2713 | N | ALA | A | 288 | 120.638 | 253.930 | 7.100 | 1.00 | 50.00 | AAAA | N |
| ATOM | 2714 | CA | ALA | A | 288 | 119.944 | 255.153 | 6.660 | 1.00 | 50.00 | AAAA | C |
| ATOM | 2715 | C | ALA | A | 288 | 120.759 | 256.156 | 5.837 | 1.00 | 50.00 | AAAA | C |
| ATOM | 2716 | O | ALA | A | 288 | 121.852 | 256.563 | 6.211 | 1.00 | 50.00 | AAAA | O |
| ATOM | 2717 | CB | ALA | A | 288 | 119.374 | 255.890 | 7.872 | 1.00 | 50.00 | AAAA | C |
| ATOM | 2718 | H | ALA | A | 288 | 121.003 | 253.858 | 8.028 | 0.00 | 0.00 | AAAA | H |
| ATOM | 2719 | N | SER | A | 289 | 120.161 | 256.545 | 4.691 | 1.00 | 50.00 | AAAA | N |
| ATOM | 2720 | CA | SER | A | 289 | 120.817 | 257.480 | 3.770 | 1.00 | 50.00 | AAAA | C |
| ATOM | 2721 | C | SER | A | 289 | 120.480 | 258.954 | 3.998 | 1.00 | 50.00 | AAAA | C |
| ATOM | 2722 | O | SER | A | 289 | 121.348 | 259.812 | 3.901 | 1.00 | 50.00 | AAAA | O |
| ATOM | 2723 | CB | SER | A | 289 | 120.538 | 257.053 | 2.314 | 1.00 | 50.00 | AAAA | C |
| ATOM | 2724 | OG | SER | A | 289 | 121.111 | 257.967 | 1.367 | 1.00 | 50.00 | AAAA | O |
| ATOM | 2725 | H | SER | A | 289 | 119.259 | 256.173 | 4.479 | 0.00 | 0.00 | AAAA | H |
| ATOM | 2726 | HG | SER | A | 289 | 120.913 | 257.629 | 0.501 | 0.00 | 0.00 | AAAA | H |
| ATOM | 2727 | N | ASN | A | 290 | 119.186 | 259.218 | 4.310 | 1.00 | 50.00 | AAAA | N |
| ATOM | 2728 | CA | ASN | A | 290 | 118.741 | 260.619 | 4.421 | 1.00 | 50.00 | AAAA | C |
| ATOM | 2729 | C | ASN | A | 290 | 119.413 | 261.361 | 5.559 | 1.00 | 50.00 | AAAA | C |
| ATOM | 2730 | O | ASN | A | 290 | 120.237 | 262.243 | 5.370 | 1.00 | 50.00 | AAAA | O |
| ATOM | 2731 | CB | ASN | A | 290 | 117.211 | 260.703 | 4.572 | 1.00 | 50.00 | AAAA | C |
| ATOM | 2732 | CG | ASN | A | 290 | 116.738 | 262.147 | 4.528 | 1.00 | 50.00 | AAAA | C |
| ATOM | 2733 | ND2 | ASN | A | 290 | 116.038 | 262.530 | 5.609 | 1.00 | 50.00 | AAAA | N |
| ATOM | 2734 | OD1 | ASN | A | 290 | 116.998 | 262.878 | 3.585 | 1.00 | 50.00 | AAAA | O |
| ATOM | 2735 | H | ASN | A | 290 | 118.539 | 258.460 | 4.398 | 0.00 | 0.00 | AAAA | H |
| ATOM | 2736 | 1HD2 | ASN | A | 290 | 115.734 | 263.483 | 5.676 | 0.00 | 0.00 | AAAA | H |
| ATOM | 2737 | 2HD2 | ASN | A | 290 | 115.809 | 261.912 | 6.360 | 0.00 | 0.00 | AAAA | H |
| ATOM | 2738 | N | LYS | A | 291 | 119.042 | 260.902 | 6.767 | 1.00 | 50.00 | AAAA | N |
| ATOM | 2739 | CA | LYS | A | 291 | 119.730 | 261.374 | 7.966 | 1.00 | 50.00 | AAAA | C |
| ATOM | 2740 | C | LYS | A | 291 | 121.122 | 260.784 | 8.172 | 1.00 | 50.00 | AAAA | C |
| ATOM | 2741 | O | LYS | A | 291 | 121.692 | 260.907 | 9.249 | 1.00 | 50.00 | AAAA | O |
| ATOM | 2742 | CB | LYS | A | 291 | 118.842 | 261.111 | 9.185 | 1.00 | 50.00 | AAAA | C |
| ATOM | 2743 | CG | LYS | A | 291 | 117.510 | 261.872 | 9.158 | 1.00 | 50.00 | AAAA | C |
| ATOM | 2744 | CD | LYS | A | 291 | 117.681 | 263.389 | 9.273 | 1.00 | 50.00 | AAAA | C |
| ATOM | 2745 | CE | LYS | A | 291 | 118.312 | 263.803 | 10.604 | 1.00 | 50.00 | AAAA | C |
| ATOM | 2746 | NZ | LYS | A | 291 | 118.453 | 265.266 | 10.663 | 1.00 | 50.00 | AAAA | N |
| ATOM | 2747 | H | LYS | A | 291 | 118.341 | 260.196 | 6.859 | 0.00 | 0.00 | AAAA | H |
| ATOM | 2748 | 1HZ | LYS | A | 291 | 118.859 | 265.530 | 11.583 | 0.00 | 0.00 | AAAA | H |
| ATOM | 2749 | 2HZ | LYS | A | 291 | 117.522 | 265.717 | 10.559 | 0.00 | 0.00 | AAAA | H |
| ATOM | 2750 | 3HZ | LYS | A | 291 | 119.085 | 265.589 | 9.904 | 0.00 | 0.00 | AAAA | H |
| ATOM | 2751 | N | ASN | A | 292 | 121.628 | 260.134 | 7.089 | 1.00 | 50.00 | AAAA | N |
| ATOM | 2752 | CA | ASN | A | 292 | 122.971 | 259.544 | 7.023 | 1.00 | 50.00 | AAAA | C |
| ATOM | 2753 | C | ASN | A | 292 | 123.481 | 258.980 | 8.337 | 1.00 | 50.00 | AAAA | C |
| ATOM | 2754 | O | ASN | A | 292 | 124.441 | 259.446 | 8.938 | 1.00 | 50.00 | AAAA | O |
| ATOM | 2755 | CB | ASN | A | 292 | 123.964 | 260.504 | 6.337 | 1.00 | 50.00 | AAAA | C |
| ATOM | 2756 | CG | ASN | A | 292 | 125.265 | 259.796 | 5.962 | 1.00 | 50.00 | AAAA | C |
| ATOM | 2757 | ND2 | ASN | A | 292 | 125.585 | 259.874 | 4.660 | 1.00 | 50.00 | AAAA | N |
| ATOM | 2758 | OD1 | ASN | A | 292 | 125.961 | 259.223 | 6.787 | 1.00 | 50.00 | AAAA | O |
| ATOM | 2759 | H | ASN | A | 292 | 121.116 | 260.130 | 6.234 | 0.00 | 0.00 | AAAA | H |
| ATOM | 2760 | 1HD2 | ASN | A | 292 | 126.457 | 259.483 | 4.368 | 0.00 | 0.00 | AAAA | H |
| ATOM | 2761 | 2HD2 | ASN | A | 292 | 124.992 | 260.309 | 3.983 | 0.00 | 0.00 | AAAA | H |
| ATOM | 2762 | N | ARG | A | 293 | 122.745 | 257.947 | 8.760 | 1.00 | 50.00 | AAAA | N |
| ATOM | 2763 | CA | ARG | A | 293 | 123.154 | 257.300 | 9.994 | 1.00 | 50.00 | AAAA | C |
| ATOM | 2764 | C | ARG | A | 293 | 122.935 | 255.806 | 9.914 | 1.00 | 50.00 | AAAA | C |
| ATOM | 2765 | O | ARG | A | 293 | 122.171 | 255.305 | 9.099 | 1.00 | 50.00 | AAAA | O |
| ATOM | 2766 | CB | ARG | A | 293 | 122.430 | 257.919 | 11.199 | 1.00 | 50.00 | AAAA | C |
| ATOM | 2767 | CG | ARG | A | 293 | 120.904 | 257.801 | 11.156 | 1.00 | 50.00 | AAAA | C |
| ATOM | 2768 | CD | ARG | A | 293 | 120.239 | 258.410 | 12.390 | 1.00 | 50.00 | AAAA | C |
| ATOM | 2769 | NE | ARG | A | 293 | 118.794 | 258.205 | 12.350 | 1.00 | 50.00 | AAAA | N |
| ATOM | 2770 | CZ | ARG | A | 293 | 117.996 | 258.681 | 13.328 | 1.00 | 50.00 | AAAA | C |
| ATOM | 2771 | NH1 | ARG | A | 293 | 118.484 | 259.362 | 14.362 | 1.00 | 50.00 | AAAA | N |
| ATOM | 2772 | NH2 | ARG | A | 293 | 116.689 | 258.463 | 13.255 | 1.00 | 50.00 | AAAA | N |
| ATOM | 2773 | H | ARG | A | 293 | 121.951 | 257.632 | 8.244 | 0.00 | 0.00 | AAAA | H |
| ATOM | 2774 | HE | ARG | A | 293 | 118.388 | 257.685 | 11.598 | 0.00 | 0.00 | AAAA | H |
| ATOM | 2775 | 1HH1 | ARG | A | 293 | 117.868 | 259.713 | 15.067 | 0.00 | 0.00 | AAAA | H |
| ATOM | 2776 | 2HH1 | ARG | A | 293 | 119.468 | 259.528 | 14.438 | 0.00 | 0.00 | AAAA | H |
| ATOM | 2777 | 1HH2 | ARG | A | 293 | 116.078 | 258.815 | 13.964 | 0.00 | 0.00 | AAAA | H |
| ATOM | 2778 | 2HH2 | ARG | A | 293 | 116.318 | 257.941 | 12.485 | 0.00 | 0.00 | AAAA | H |
| ATOM | 2779 | N | GLY | A | 294 | 123.648 | 255.108 | 10.807 | 1.00 | 50.00 | AAAA | N |
| ATOM | 2780 | CA | GLY | A | 294 | 123.362 | 253.682 | 10.899 | 1.00 | 50.00 | AAAA | C |
| ATOM | 2781 | C | GLY | A | 294 | 122.326 | 253.453 | 11.969 | 1.00 | 50.00 | AAAA | C |
| ATOM | 2782 | O | GLY | A | 294 | 122.559 | 253.739 | 13.137 | 1.00 | 50.00 | AAAA | O |
| ATOM | 2783 | H | GLY | A | 294 | 124.203 | 255.584 | 11.487 | 0.00 | 0.00 | AAAA | H |
| ATOM | 2784 | N | ILE | A | 295 | 121.166 | 252.953 | 11.516 | 1.00 | 50.00 | AAAA | N |
| ATOM | 2785 | CA | ILE | A | 295 | 120.126 | 252.731 | 12.515 | 1.00 | 50.00 | AAAA | C |
| ATOM | 2786 | C | ILE | A | 295 | 120.440 | 251.591 | 13.467 | 1.00 | 50.00 | AAAA | C |
| ATOM | 2787 | O | ILE | A | 295 | 120.621 | 250.436 | 13.099 | 1.00 | 50.00 | AAAA | O |
| ATOM | 2788 | CB | ILE | A | 295 | 118.741 | 252.564 | 11.882 | 1.00 | 50.00 | AAAA | C |
| ATOM | 2789 | CG1 | ILE | A | 295 | 118.487 | 253.631 | 10.814 | 1.00 | 50.00 | AAAA | C |
| ATOM | 2790 | CG2 | ILE | A | 295 | 117.663 | 252.630 | 12.979 | 1.00 | 50.00 | AAAA | C |
| ATOM | 2791 | CD1 | ILE | A | 295 | 117.150 | 253.474 | 10.083 | 1.00 | 50.00 | AAAA | C |
| ATOM | 2792 | H | ILE | A | 295 | 121.042 | 252.662 | 10.568 | 0.00 | 0.00 | AAAA | H |
| ATOM | 2793 | N | ILE | A | 296 | 120.524 | 252.021 | 14.735 | 1.00 | 50.00 | AAAA | N |
| ATOM | 2794 | CA | ILE | A | 296 | 120.838 | 251.062 | 15.787 | 1.00 | 50.00 | AAAA | C |
| ATOM | 2795 | C | ILE | A | 296 | 119.598 | 250.402 | 16.373 | 1.00 | 50.00 | AAAA | C |
| ATOM | 2796 | O | ILE | A | 296 | 118.554 | 251.018 | 16.570 | 1.00 | 50.00 | AAAA | O |
| ATOM | 2797 | CB | ILE | A | 296 | 121.685 | 251.736 | 16.881 | 1.00 | 50.00 | AAAA | C |
| ATOM | 2798 | CG1 | ILE | A | 296 | 120.947 | 252.899 | 17.563 | 1.00 | 50.00 | AAAA | C |
| ATOM | 2799 | CG2 | ILE | A | 296 | 123.013 | 252.218 | 16.282 | 1.00 | 50.00 | AAAA | C |
| ATOM | 2800 | CD1 | ILE | A | 296 | 121.700 | 253.503 | 18.751 | 1.00 | 50.00 | AAAA | C |
| ATOM | 2801 | H | ILE | A | 296 | 120.298 | 252.967 | 14.964 | 0.00 | 0.00 | AAAA | H |
| ATOM | 2802 | N | THR | A | 297 | 119.768 | 249.101 | 16.662 | 1.00 | 50.00 | AAAA | N |
| ATOM | 2803 | CA | THR | A | 297 | 118.700 | 248.460 | 17.425 | 1.00 | 50.00 | AAAA | C |
| ATOM | 2804 | C | THR | A | 297 | 119.096 | 248.195 | 18.831 | 1.00 | 50.00 | AAAA | C |
| ATOM | 2805 | O | THR | A | 297 | 120.022 | 247.447 | 19.130 | 1.00 | 50.00 | AAAA | O |
| ATOM | 2806 | CB | THR | A | 297 | 118.260 | 247.122 | 16.877 | 1.00 | 50.00 | AAAA | C |
| ATOM | 2807 | CG2 | THR | A | 297 | 116.972 | 247.224 | 16.076 | 1.00 | 50.00 | AAAA | C |
| ATOM | 2808 | OG1 | THR | A | 297 | 119.335 | 246.525 | 16.164 | 1.00 | 50.00 | AAAA | O |
| ATOM | 2809 | H | THR | A | 297 | 120.631 | 248.632 | 16.464 | 0.00 | 0.00 | AAAA | H |
| ATOM | 2810 | HG1 | THR | A | 297 | 119.082 | 245.624 | 16.021 | 0.00 | 0.00 | AAAA | H |
| ATOM | 2811 | N | THR | A | 298 | 118.283 | 248.818 | 19.686 | 1.00 | 50.00 | AAAA | N |
| ATOM | 2812 | CA | THR | A | 298 | 118.392 | 248.452 | 21.084 | 1.00 | 50.00 | AAAA | C |
| ATOM | 2813 | C | THR | A | 298 | 118.019 | 246.997 | 21.281 | 1.00 | 50.00 | AAAA | C |
| ATOM | 2814 | O | THR | A | 298 | 117.209 | 246.427 | 20.559 | 1.00 | 50.00 | AAAA | O |
| ATOM | 2815 | CB | THR | A | 298 | 117.519 | 249.379 | 21.933 | 1.00 | 50.00 | AAAA | C |
| ATOM | 2816 | CG2 | THR | A | 298 | 117.956 | 250.836 | 21.776 | 1.00 | 50.00 | AAAA | C |
| ATOM | 2817 | OG1 | THR | A | 298 | 116.141 | 249.247 | 21.578 | 1.00 | 50.00 | AAAA | O |
| ATOM | 2818 | H | THR | A | 298 | 117.520 | 249.373 | 19.361 | 0.00 | 0.00 | AAAA | H |
| ATOM | 2819 | HG1 | THR | A | 298 | 115.647 | 249.762 | 22.202 | 0.00 | 0.00 | AAAA | H |
| ATOM | 2820 | N | PHE | A | 299 | 118.682 | 246.415 | 22.288 | 1.00 | 50.00 | AAAA | N |
| ATOM | 2821 | CA | PHE | A | 299 | 118.382 | 245.031 | 22.636 | 1.00 | 50.00 | AAAA | C |
| ATOM | 2822 | C | PHE | A | 299 | 116.903 | 244.800 | 22.908 | 1.00 | 50.00 | AAAA | C |
| ATOM | 2823 | O | PHE | A | 299 | 116.329 | 243.803 | 22.493 | 1.00 | 50.00 | AAAA | O |
| ATOM | 2824 | CB | PHE | A | 299 | 119.256 | 244.609 | 23.819 | 1.00 | 50.00 | AAAA | C |
| ATOM | 2825 | CG | PHE | A | 299 | 120.711 | 244.510 | 23.423 | 1.00 | 50.00 | AAAA | C |
| ATOM | 2826 | CD1 | PHE | A | 299 | 121.184 | 243.324 | 22.819 | 1.00 | 50.00 | AAAA | C |
| ATOM | 2827 | CD2 | PHE | A | 299 | 121.575 | 245.599 | 23.673 | 1.00 | 50.00 | AAAA | C |
| ATOM | 2828 | CE1 | PHE | A | 299 | 122.543 | 243.222 | 22.465 | 1.00 | 50.00 | AAAA | C |
| ATOM | 2829 | CE2 | PHE | A | 299 | 122.935 | 245.499 | 23.318 | 1.00 | 50.00 | AAAA | C |
| ATOM | 2830 | CZ | PHE | A | 299 | 123.405 | 244.310 | 22.720 | 1.00 | 50.00 | AAAA | C |
| ATOM | 2831 | H | PHE | A | 299 | 119.377 | 246.938 | 22.778 | 0.00 | 0.00 | AAAA | H |
| ATOM | 2832 | N | SER | A | 300 | 116.301 | 245.819 | 23.556 | 1.00 | 50.00 | AAAA | N |
| ATOM | 2833 | CA | SER | A | 300 | 114.859 | 245.804 | 23.809 | 1.00 | 50.00 | AAAA | C |
| ATOM | 2834 | C | SER | A | 300 | 113.902 | 245.827 | 22.624 | 1.00 | 50.00 | AAAA | C |
| ATOM | 2835 | O | SER | A | 300 | 112.693 | 245.935 | 22.787 | 1.00 | 50.00 | AAAA | O |
| ATOM | 2836 | CB | SER | A | 300 | 114.517 | 246.904 | 24.820 | 1.00 | 50.00 | AAAA | C |
| ATOM | 2837 | OG | SER | A | 300 | 115.097 | 248.149 | 24.414 | 1.00 | 50.00 | AAAA | O |
| ATOM | 2838 | H | SER | A | 300 | 116.837 | 246.622 | 23.812 | 0.00 | 0.00 | AAAA | H |
| ATOM | 2839 | HG | SER | A | 300 | 114.556 | 248.828 | 24.796 | 0.00 | 0.00 | AAAA | H |
| ATOM | 2840 | N | ASN | A | 301 | 114.484 | 245.710 | 21.423 | 1.00 | 50.00 | AAAA | N |
| ATOM | 2841 | CA | ASN | A | 301 | 113.659 | 245.629 | 20.227 | 1.00 | 50.00 | AAAA | C |
| ATOM | 2842 | C | ASN | A | 301 | 114.171 | 244.543 | 19.304 | 1.00 | 50.00 | AAAA | C |
| ATOM | 2843 | O | ASN | A | 301 | 115.181 | 244.701 | 18.626 | 1.00 | 50.00 | AAAA | O |
| ATOM | 2844 | CB | ASN | A | 301 | 113.615 | 246.980 | 19.500 | 1.00 | 50.00 | AAAA | C |
| ATOM | 2845 | CG | ASN | A | 301 | 112.940 | 248.036 | 20.359 | 1.00 | 50.00 | AAAA | C |
| ATOM | 2846 | ND2 | ASN | A | 301 | 111.621 | 247.846 | 20.522 | 1.00 | 50.00 | AAAA | N |
| ATOM | 2847 | OD1 | ASN | A | 301 | 113.553 | 248.970 | 20.861 | 1.00 | 50.00 | AAAA | O |
| ATOM | 2848 | H | ASN | A | 301 | 115.477 | 245.668 | 21.350 | 0.00 | 0.00 | AAAA | H |
| ATOM | 2849 | 1HD2 | ASN | A | 301 | 111.104 | 248.473 | 21.102 | 0.00 | 0.00 | AAAA | H |
| ATOM | 2850 | 2HD2 | ASN | A | 301 | 111.161 | 247.076 | 20.081 | 0.00 | 0.00 | AAAA | H |
| ATOM | 2851 | N | GLY | A | 302 | 113.399 | 243.434 | 19.300 | 1.00 | 50.00 | AAAA | N |
| ATOM | 2852 | CA | GLY | A | 302 | 113.713 | 242.298 | 18.423 | 1.00 | 50.00 | AAAA | C |
| ATOM | 2853 | C | GLY | A | 302 | 113.687 | 242.626 | 16.939 | 1.00 | 50.00 | AAAA | C |
| ATOM | 2854 | O | GLY | A | 302 | 114.453 | 242.114 | 16.131 | 1.00 | 50.00 | AAAA | O |
| ATOM | 2855 | H | GLY | A | 302 | 112.620 | 243.407 | 19.923 | 0.00 | 0.00 | AAAA | H |
| ATOM | 2856 | N | CYS | A | 303 | 112.772 | 243.563 | 16.628 | 1.00 | 50.00 | AAAA | N |
| ATOM | 2857 | CA | CYS | A | 303 | 112.822 | 244.192 | 15.312 | 1.00 | 50.00 | AAAA | C |
| ATOM | 2858 | C | CYS | A | 303 | 112.514 | 245.668 | 15.408 | 1.00 | 50.00 | AAAA | C |
| ATOM | 2859 | O | CYS | A | 303 | 111.735 | 246.127 | 16.235 | 1.00 | 50.00 | AAAA | O |
| ATOM | 2860 | CB | CYS | A | 303 | 111.830 | 243.618 | 14.301 | 1.00 | 50.00 | AAAA | C |
| ATOM | 2861 | SG | CYS | A | 303 | 111.264 | 241.936 | 14.582 | 1.00 | 50.00 | AAAA | S |
| ATOM | 2862 | H | CYS | A | 303 | 112.188 | 243.941 | 17.341 | 0.00 | 0.00 | AAAA | H |
| ATOM | 2863 | N | ASP | A | 304 | 113.152 | 246.377 | 14.474 | 1.00 | 50.00 | AAAA | N |
| ATOM | 2864 | CA | ASP | A | 304 | 112.923 | 247.807 | 14.341 | 1.00 | 50.00 | AAAA | C |
| ATOM | 2865 | C | ASP | A | 304 | 111.986 | 248.098 | 13.182 | 1.00 | 50.00 | AAAA | C |
| ATOM | 2866 | O | ASP | A | 304 | 112.020 | 247.466 | 12.135 | 1.00 | 50.00 | AAAA | O |
| ATOM | 2867 | CB | ASP | A | 304 | 114.269 | 248.502 | 14.124 | 1.00 | 50.00 | AAAA | C |
| ATOM | 2868 | CG | ASP | A | 304 | 114.284 | 249.976 | 14.484 | 1.00 | 50.00 | AAAA | C |
| ATOM | 2869 | OD1 | ASP | A | 304 | 113.310 | 250.672 | 14.237 | 1.00 | 50.00 | AAAA | O |
| ATOM | 2870 | OD2 | ASP | A | 304 | 115.287 | 250.433 | 15.022 | 1.00 | 50.00 | AAAA | O |
| ATOM | 2871 | H | ASP | A | 304 | 113.712 | 245.888 | 13.811 | 0.00 | 0.00 | AAAA | H |
| ATOM | 2872 | N | TYR | A | 305 | 111.151 | 249.111 | 13.422 | 1.00 | 50.00 | AAAA | N |
| ATOM | 2873 | CA | TYR | A | 305 | 110.346 | 249.664 | 12.345 | 1.00 | 50.00 | AAAA | C |
| ATOM | 2874 | C | TYR | A | 305 | 111.022 | 250.844 | 11.700 | 1.00 | 50.00 | AAAA | C |
| ATOM | 2875 | O | TYR | A | 305 | 111.731 | 251.606 | 12.333 | 1.00 | 50.00 | AAAA | O |
| ATOM | 2876 | CB | TYR | A | 305 | 109.069 | 250.188 | 12.941 | 1.00 | 50.00 | AAAA | C |
| ATOM | 2877 | CG | TYR | A | 305 | 108.037 | 250.477 | 11.894 | 1.00 | 50.00 | AAAA | C |
| ATOM | 2878 | CD1 | TYR | A | 305 | 107.291 | 249.412 | 11.349 | 1.00 | 50.00 | AAAA | C |
| ATOM | 2879 | CD2 | TYR | A | 305 | 107.855 | 251.814 | 11.511 | 1.00 | 50.00 | AAAA | C |
| ATOM | 2880 | CE1 | TYR | A | 305 | 106.263 | 249.713 | 10.447 | 1.00 | 50.00 | AAAA | C |
| ATOM | 2881 | CE2 | TYR | A | 305 | 106.847 | 252.105 | 10.590 | 1.00 | 50.00 | AAAA | C |
| ATOM | 2882 | CZ | TYR | A | 305 | 106.023 | 251.063 | 10.132 | 1.00 | 50.00 | AAAA | C |
| ATOM | 2883 | OH | TYR | A | 305 | 104.905 | 251.379 | 9.390 | 1.00 | 50.00 | AAAA | O |
| ATOM | 2884 | H | TYR | A | 305 | 111.136 | 249.570 | 14.309 | 0.00 | 0.00 | AAAA | H |
| ATOM | 2885 | HH | TYR | A | 305 | 104.805 | 252.326 | 9.327 | 0.00 | 0.00 | AAAA | H |
| ATOM | 2886 | N | VAL | A | 306 | 110.714 | 251.010 | 10.417 | 1.00 | 50.00 | AAAA | N |
| ATOM | 2887 | CA | VAL | A | 306 | 111.225 | 252.217 | 9.805 | 1.00 | 50.00 | AAAA | C |
| ATOM | 2888 | C | VAL | A | 306 | 110.210 | 252.889 | 8.893 | 1.00 | 50.00 | AAAA | C |
| ATOM | 2889 | O | VAL | A | 306 | 109.953 | 252.525 | 7.751 | 1.00 | 50.00 | AAAA | O |
| ATOM | 2890 | CB | VAL | A | 306 | 112.567 | 251.877 | 9.166 | 1.00 | 50.00 | AAAA | C |
| ATOM | 2891 | CG1 | VAL | A | 306 | 112.409 | 250.927 | 8.008 | 1.00 | 50.00 | AAAA | C |
| ATOM | 2892 | CG2 | VAL | A | 306 | 113.367 | 253.098 | 8.782 | 1.00 | 50.00 | AAAA | C |
| ATOM | 2893 | H | VAL | A | 306 | 110.248 | 250.303 | 9.897 | 0.00 | 0.00 | AAAA | H |
| ATOM | 2894 | N | SER | A | 307 | 109.610 | 253.916 | 9.502 | 1.00 | 50.00 | AAAA | N |
| ATOM | 2895 | CA | SER | A | 307 | 108.823 | 254.811 | 8.667 | 1.00 | 50.00 | AAAA | C |
| ATOM | 2896 | C | SER | A | 307 | 109.656 | 256.029 | 8.315 | 1.00 | 50.00 | AAAA | C |
| ATOM | 2897 | O | SER | A | 307 | 110.843 | 256.086 | 8.613 | 1.00 | 50.00 | AAAA | O |
| ATOM | 2898 | CB | SER | A | 307 | 107.524 | 255.188 | 9.385 | 1.00 | 50.00 | AAAA | C |
| ATOM | 2899 | OG | SER | A | 307 | 107.806 | 255.700 | 10.691 | 1.00 | 50.00 | AAAA | O |
| ATOM | 2900 | H | SER | A | 307 | 109.815 | 254.164 | 10.448 | 0.00 | 0.00 | AAAA | H |
| ATOM | 2901 | HG | SER | A | 307 | 106.985 | 255.752 | 11.162 | 0.00 | 0.00 | AAAA | H |
| ATOM | 2902 | N | ASN | A | 308 | 108.982 | 257.014 | 7.688 | 1.00 | 50.00 | AAAA | N |
| ATOM | 2903 | CA | ASN | A | 308 | 109.688 | 258.245 | 7.307 | 1.00 | 50.00 | AAAA | C |
| ATOM | 2904 | C | ASN | A | 308 | 110.389 | 258.968 | 8.440 | 1.00 | 50.00 | AAAA | C |
| ATOM | 2905 | O | ASN | A | 308 | 111.473 | 259.515 | 8.281 | 1.00 | 50.00 | AAAA | O |
| ATOM | 2906 | CB | ASN | A | 308 | 108.707 | 259.183 | 6.647 | 1.00 | 50.00 | AAAA | C |
| ATOM | 2907 | CG | ASN | A | 308 | 109.316 | 260.451 | 6.070 | 1.00 | 50.00 | AAAA | C |
| ATOM | 2908 | ND2 | ASN | A | 308 | 109.101 | 260.592 | 4.755 | 1.00 | 50.00 | AAAA | N |
| ATOM | 2909 | OD1 | ASN | A | 308 | 109.848 | 261.302 | 6.770 | 1.00 | 50.00 | AAAA | O |
| ATOM | 2910 | H | ASN | A | 308 | 108.020 | 256.882 | 7.447 | 0.00 | 0.00 | AAAA | H |
| ATOM | 2911 | 1HD2 | ASN | A | 308 | 109.346 | 261.448 | 4.304 | 0.00 | 0.00 | AAAA | H |
| ATOM | 2912 | 2HD2 | ASN | A | 308 | 108.690 | 259.852 | 4.225 | 0.00 | 0.00 | AAAA | H |
| ATOM | 2913 | N | LYS | A | 309 | 109.713 | 258.930 | 9.606 | 1.00 | 50.00 | AAAA | N |
| ATOM | 2914 | CA | LYS | A | 309 | 110.293 | 259.618 | 10.759 | 1.00 | 50.00 | AAAA | C |
| ATOM | 2915 | C | LYS | A | 309 | 111.717 | 259.189 | 11.117 | 1.00 | 50.00 | AAAA | C |
| ATOM | 2916 | O | LYS | A | 309 | 112.530 | 259.981 | 11.577 | 1.00 | 50.00 | AAAA | O |
| ATOM | 2917 | CB | LYS | A | 309 | 109.341 | 259.488 | 11.954 | 1.00 | 50.00 | AAAA | C |
| ATOM | 2918 | CG | LYS | A | 309 | 109.746 | 260.322 | 13.177 | 1.00 | 50.00 | AAAA | C |
| ATOM | 2919 | CD | LYS | A | 309 | 109.807 | 261.826 | 12.888 | 1.00 | 50.00 | AAAA | C |
| ATOM | 2920 | CE | LYS | A | 309 | 110.359 | 262.643 | 14.060 | 1.00 | 50.00 | AAAA | C |
| ATOM | 2921 | NZ | LYS | A | 309 | 111.778 | 262.317 | 14.289 | 1.00 | 50.00 | AAAA | N |
| ATOM | 2922 | H | LYS | A | 309 | 108.812 | 258.500 | 9.656 | 0.00 | 0.00 | AAAA | H |
| ATOM | 2923 | 1HZ | LYS | A | 309 | 112.135 | 262.867 | 15.096 | 0.00 | 0.00 | AAAA | H |
| ATOM | 2924 | 2HZ | LYS | A | 309 | 112.330 | 262.548 | 13.439 | 0.00 | 0.00 | AAAA | H |
| ATOM | 2925 | 3HZ | LYS | A | 309 | 111.870 | 261.301 | 14.495 | 0.00 | 0.00 | AAAA | H |
| ATOM | 2926 | N | GLU | A | 310 | 111.975 | 257.891 | 10.867 | 1.00 | 50.00 | AAAA | N |
| ATOM | 2927 | CA | GLU | A | 310 | 113.322 | 257.395 | 11.142 | 1.00 | 50.00 | AAAA | C |
| ATOM | 2928 | C | GLU | A | 310 | 114.318 | 257.548 | 10.000 | 1.00 | 50.00 | AAAA | C |
| ATOM | 2929 | O | GLU | A | 310 | 115.522 | 257.636 | 10.210 | 1.00 | 50.00 | AAAA | O |
| ATOM | 2930 | CB | GLU | A | 310 | 113.244 | 255.949 | 11.623 | 1.00 | 50.00 | AAAA | C |
| ATOM | 2931 | CG | GLU | A | 310 | 112.465 | 255.851 | 12.938 | 1.00 | 50.00 | AAAA | C |
| ATOM | 2932 | CD | GLU | A | 310 | 112.399 | 254.418 | 13.437 | 1.00 | 50.00 | AAAA | C |
| ATOM | 2933 | OE1 | GLU | A | 310 | 113.453 | 253.802 | 13.605 | 1.00 | 50.00 | AAAA | O |
| ATOM | 2934 | OE2 | GLU | A | 310 | 111.287 | 253.947 | 13.672 | 1.00 | 50.00 | AAAA | O |
| ATOM | 2935 | H | GLU | A | 310 | 111.295 | 257.326 | 10.401 | 0.00 | 0.00 | AAAA | H |
| ATOM | 2936 | N | VAL | A | 311 | 113.748 | 257.587 | 8.781 | 1.00 | 50.00 | AAAA | N |
| ATOM | 2937 | CA | VAL | A | 311 | 114.524 | 257.845 | 7.569 | 1.00 | 50.00 | AAAA | C |
| ATOM | 2938 | C | VAL | A | 311 | 113.576 | 258.170 | 6.423 | 1.00 | 50.00 | AAAA | C |
| ATOM | 2939 | O | VAL | A | 311 | 112.440 | 257.725 | 6.430 | 1.00 | 50.00 | AAAA | O |
| ATOM | 2940 | CB | VAL | A | 311 | 115.436 | 256.645 | 7.228 | 1.00 | 50.00 | AAAA | C |
| ATOM | 2941 | CG1 | VAL | A | 311 | 114.682 | 255.423 | 6.720 | 1.00 | 50.00 | AAAA | C |
| ATOM | 2942 | CG2 | VAL | A | 311 | 116.515 | 257.041 | 6.229 | 1.00 | 50.00 | AAAA | C |
| ATOM | 2943 | H | VAL | A | 311 | 112.757 | 257.473 | 8.707 | 0.00 | 0.00 | AAAA | H |
| ATOM | 2944 | N | ASP | A | 312 | 114.070 | 258.951 | 5.444 | 1.00 | 50.00 | AAAA | N |
| ATOM | 2945 | CA | ASP | A | 312 | 113.280 | 259.109 | 4.217 | 1.00 | 50.00 | AAAA | C |
| ATOM | 2946 | C | ASP | A | 312 | 113.876 | 258.311 | 3.053 | 1.00 | 50.00 | AAAA | C |
| ATOM | 2947 | O | ASP | A | 312 | 113.206 | 257.942 | 2.101 | 1.00 | 50.00 | AAAA | O |
| ATOM | 2948 | CB | ASP | A | 312 | 113.142 | 260.602 | 3.888 | 1.00 | 50.00 | AAAA | C |
| ATOM | 2949 | CG | ASP | A | 312 | 111.990 | 260.919 | 2.938 | 1.00 | 50.00 | AAAA | C |
| ATOM | 2950 | OD1 | ASP | A | 312 | 111.801 | 260.226 | 1.940 | 1.00 | 50.00 | AAAA | O |
| ATOM | 2951 | OD2 | ASP | A | 312 | 111.292 | 261.900 | 3.184 | 1.00 | 50.00 | AAAA | O |
| ATOM | 2952 | H | ASP | A | 312 | 114.994 | 259.321 | 5.514 | 0.00 | 0.00 | AAAA | H |
| ATOM | 2953 | N | THR | A | 313 | 115.184 | 258.029 | 3.171 | 1.00 | 50.00 | AAAA | N |
| ATOM | 2954 | CA | THR | A | 313 | 115.834 | 257.253 | 2.112 | 1.00 | 50.00 | AAAA | C |
| ATOM | 2955 | C | THR | A | 313 | 116.680 | 256.148 | 2.724 | 1.00 | 50.00 | AAAA | C |
| ATOM | 2956 | O | THR | A | 313 | 117.655 | 256.424 | 3.406 | 1.00 | 50.00 | AAAA | O |
| ATOM | 2957 | CB | THR | A | 313 | 116.701 | 258.169 | 1.225 | 1.00 | 50.00 | AAAA | C |
| ATOM | 2958 | CG2 | THR | A | 313 | 115.876 | 259.209 | 0.462 | 1.00 | 50.00 | AAAA | C |
| ATOM | 2959 | OG1 | THR | A | 313 | 117.687 | 258.837 | 2.017 | 1.00 | 50.00 | AAAA | O |
| ATOM | 2960 | H | THR | A | 313 | 115.719 | 258.350 | 3.951 | 0.00 | 0.00 | AAAA | H |
| ATOM | 2961 | HG1 | THR | A | 313 | 118.095 | 259.492 | 1.465 | 0.00 | 0.00 | AAAA | H |
| ATOM | 2962 | N | VAL | A | 314 | 116.259 | 254.898 | 2.485 | 1.00 | 50.00 | AAAA | N |
| ATOM | 2963 | CA | VAL | A | 314 | 116.975 | 253.784 | 3.113 | 1.00 | 50.00 | AAAA | C |
| ATOM | 2964 | C | VAL | A | 314 | 117.824 | 252.968 | 2.138 | 1.00 | 50.00 | AAAA | C |
| ATOM | 2965 | O | VAL | A | 314 | 117.504 | 252.830 | 0.962 | 1.00 | 50.00 | AAAA | O |
| ATOM | 2966 | CB | VAL | A | 314 | 115.959 | 252.912 | 3.890 | 1.00 | 50.00 | AAAA | C |
| ATOM | 2967 | CG1 | VAL | A | 314 | 114.909 | 252.293 | 2.959 | 1.00 | 50.00 | AAAA | C |
| ATOM | 2968 | CG2 | VAL | A | 314 | 116.607 | 251.878 | 4.821 | 1.00 | 50.00 | AAAA | C |
| ATOM | 2969 | H | VAL | A | 314 | 115.478 | 254.740 | 1.885 | 0.00 | 0.00 | AAAA | H |
| ATOM | 2970 | N | SER | A | 315 | 118.922 | 252.426 | 2.696 | 1.00 | 50.00 | AAAA | N |
| ATOM | 2971 | CA | SER | A | 315 | 119.760 | 251.478 | 1.974 | 1.00 | 50.00 | AAAA | C |
| ATOM | 2972 | C | SER | A | 315 | 119.932 | 250.171 | 2.744 | 1.00 | 50.00 | AAAA | C |
| ATOM | 2973 | O | SER | A | 315 | 120.386 | 250.114 | 3.879 | 1.00 | 50.00 | AAAA | O |
| ATOM | 2974 | CB | SER | A | 315 | 121.100 | 252.144 | 1.631 | 1.00 | 50.00 | AAAA | C |
| ATOM | 2975 | OG | SER | A | 315 | 121.922 | 251.278 | 0.838 | 1.00 | 50.00 | AAAA | O |
| ATOM | 2976 | H | SER | A | 315 | 119.167 | 252.652 | 3.637 | 0.00 | 0.00 | AAAA | H |
| ATOM | 2977 | HG | SER | A | 315 | 122.719 | 251.762 | 0.664 | 0.00 | 0.00 | AAAA | H |
| ATOM | 2978 | N | VAL | A | 316 | 119.525 | 249.090 | 2.064 | 1.00 | 50.00 | AAAA | N |
| ATOM | 2979 | CA | VAL | A | 316 | 119.707 | 247.769 | 2.654 | 1.00 | 50.00 | AAAA | C |
| ATOM | 2980 | C | VAL | A | 316 | 120.491 | 246.849 | 1.727 | 1.00 | 50.00 | AAAA | C |
| ATOM | 2981 | O | VAL | A | 316 | 119.954 | 246.134 | 0.888 | 1.00 | 50.00 | AAAA | O |
| ATOM | 2982 | CB | VAL | A | 316 | 118.348 | 247.187 | 3.115 | 1.00 | 50.00 | AAAA | C |
| ATOM | 2983 | CG1 | VAL | A | 316 | 117.275 | 247.201 | 2.020 | 1.00 | 50.00 | AAAA | C |
| ATOM | 2984 | CG2 | VAL | A | 316 | 118.504 | 245.822 | 3.794 | 1.00 | 50.00 | AAAA | C |
| ATOM | 2985 | H | VAL | A | 316 | 119.093 | 249.185 | 1.173 | 0.00 | 0.00 | AAAA | H |
| ATOM | 2986 | N | GLY | A | 317 | 121.825 | 246.930 | 1.922 | 1.00 | 50.00 | AAAA | N |
| ATOM | 2987 | CA | GLY | A | 317 | 122.756 | 246.075 | 1.176 | 1.00 | 50.00 | AAAA | C |
| ATOM | 2988 | C | GLY | A | 317 | 122.525 | 245.981 | −0.324 | 1.00 | 50.00 | AAAA | C |
| ATOM | 2989 | O | GLY | A | 317 | 122.045 | 244.974 | −0.827 | 1.00 | 50.00 | AAAA | O |
| ATOM | 2990 | H | GLY | A | 317 | 122.163 | 247.520 | 2.656 | 0.00 | 0.00 | AAAA | H |
| ATOM | 2991 | N | ASN | A | 318 | 122.890 | 247.100 | −0.991 | 1.00 | 50.00 | AAAA | N |
| ATOM | 2992 | CA | ASN | A | 318 | 122.735 | 247.279 | −2.444 | 1.00 | 50.00 | AAAA | C |
| ATOM | 2993 | C | ASN | A | 318 | 121.327 | 247.631 | −2.919 | 1.00 | 50.00 | AAAA | C |
| ATOM | 2994 | O | ASN | A | 318 | 121.050 | 247.699 | −4.108 | 1.00 | 50.00 | AAAA | O |
| ATOM | 2995 | CB | ASN | A | 318 | 123.317 | 246.113 | −3.271 | 1.00 | 50.00 | AAAA | C |
| ATOM | 2996 | CG | ASN | A | 318 | 124.784 | 245.910 | −2.941 | 1.00 | 50.00 | AAAA | C |
| ATOM | 2997 | ND2 | ASN | A | 318 | 125.092 | 244.660 | −2.551 | 1.00 | 50.00 | AAAA | N |
| ATOM | 2998 | OD1 | ASN | A | 318 | 125.594 | 246.825 | −3.021 | 1.00 | 50.00 | AAAA | O |
| ATOM | 2999 | H | ASN | A | 318 | 123.231 | 247.858 | −0.435 | 0.00 | 0.00 | AAAA | H |
| ATOM | 3000 | 1HD2 | ASN | A | 318 | 126.047 | 244.442 | −2.352 | 0.00 | 0.00 | AAAA | H |
| ATOM | 3001 | 2HD2 | ASN | A | 318 | 124.404 | 243.939 | −2.464 | 0.00 | 0.00 | AAAA | H |
| ATOM | 3002 | N | THR | A | 319 | 120.446 | 247.865 | −1.929 | 1.00 | 50.00 | AAAA | N |
| ATOM | 3003 | CA | THR | A | 319 | 119.043 | 248.125 | −2.255 | 1.00 | 50.00 | AAAA | C |
| ATOM | 3004 | C | THR | A | 319 | 118.567 | 249.469 | −1.696 | 1.00 | 50.00 | AAAA | C |
| ATOM | 3005 | O | THR | A | 319 | 118.381 | 249.644 | −0.500 | 1.00 | 50.00 | AAAA | O |
| ATOM | 3006 | CB | THR | A | 319 | 118.228 | 246.922 | −1.738 | 1.00 | 50.00 | AAAA | C |
| ATOM | 3007 | CG2 | THR | A | 319 | 116.716 | 247.110 | −1.682 | 1.00 | 50.00 | AAAA | C |
| ATOM | 3008 | OG1 | THR | A | 319 | 118.528 | 245.767 | −2.524 | 1.00 | 50.00 | AAAA | O |
| ATOM | 3009 | H | THR | A | 319 | 120.724 | 247.805 | −0.971 | 0.00 | 0.00 | AAAA | H |
| ATOM | 3010 | HG1 | THR | A | 319 | 117.991 | 245.060 | −2.189 | 0.00 | 0.00 | AAAA | H |
| ATOM | 3011 | N | LEU | A | 320 | 118.393 | 250.436 | −2.615 | 1.00 | 50.00 | AAAA | N |
| ATOM | 3012 | CA | LEU | A | 320 | 118.027 | 251.774 | −2.137 | 1.00 | 50.00 | AAAA | C |
| ATOM | 3013 | C | LEU | A | 320 | 116.642 | 252.189 | −2.585 | 1.00 | 50.00 | AAAA | C |
| ATOM | 3014 | O | LEU | A | 320 | 116.303 | 252.089 | −3.757 | 1.00 | 50.00 | AAAA | O |
| ATOM | 3015 | CB | LEU | A | 320 | 119.022 | 252.841 | −2.610 | 1.00 | 50.00 | AAAA | C |
| ATOM | 3016 | CG | LEU | A | 320 | 120.395 | 252.886 | −1.930 | 1.00 | 50.00 | AAAA | C |
| ATOM | 3017 | CD1 | LEU | A | 320 | 121.320 | 251.727 | −2.311 | 1.00 | 50.00 | AAAA | C |
| ATOM | 3018 | CD2 | LEU | A | 320 | 121.077 | 254.226 | −2.202 | 1.00 | 50.00 | AAAA | C |
| ATOM | 3019 | H | LEU | A | 320 | 118.508 | 250.256 | −3.591 | 0.00 | 0.00 | AAAA | H |
| ATOM | 3020 | N | TYR | A | 321 | 115.854 | 252.661 | −1.596 | 1.00 | 50.00 | AAAA | N |
| ATOM | 3021 | CA | TYR | A | 321 | 114.479 | 253.095 | −1.857 | 1.00 | 50.00 | AAAA | C |
| ATOM | 3022 | C | TYR | A | 321 | 114.032 | 254.176 | −0.886 | 1.00 | 50.00 | AAAA | C |
| ATOM | 3023 | O | TYR | A | 321 | 114.622 | 254.397 | 0.165 | 1.00 | 50.00 | AAAA | O |
| ATOM | 3024 | CB | TYR | A | 321 | 113.469 | 251.931 | −1.790 | 1.00 | 50.00 | AAAA | C |
| ATOM | 3025 | CG | TYR | A | 321 | 113.711 | 250.912 | −2.883 | 1.00 | 50.00 | AAAA | C |
| ATOM | 3026 | CD1 | TYR | A | 321 | 113.226 | 251.159 | −4.186 | 1.00 | 50.00 | AAAA | C |
| ATOM | 3027 | CD2 | TYR | A | 321 | 114.434 | 249.744 | −2.573 | 1.00 | 50.00 | AAAA | C |
| ATOM | 3028 | CE1 | TYR | A | 321 | 113.505 | 250.233 | −5.207 | 1.00 | 50.00 | AAAA | C |
| ATOM | 3029 | CE2 | TYR | A | 321 | 114.713 | 248.825 | −3.597 | 1.00 | 50.00 | AAAA | C |
| ATOM | 3030 | CZ | TYR | A | 321 | 114.255 | 249.081 | −4.901 | 1.00 | 50.00 | AAAA | C |
| ATOM | 3031 | OH | TYR | A | 321 | 114.546 | 248.179 | −5.907 | 1.00 | 50.00 | AAAA | O |
| ATOM | 3032 | H | TYR | A | 321 | 116.215 | 252.731 | −0.667 | 0.00 | 0.00 | AAAA | H |
| ATOM | 3033 | HH | TYR | A | 321 | 115.157 | 247.523 | −5.594 | 0.00 | 0.00 | AAAA | H |
| ATOM | 3034 | N | TYR | A | 322 | 112.935 | 254.838 | −1.291 | 1.00 | 50.00 | AAAA | N |
| ATOM | 3035 | CA | TYR | A | 322 | 112.337 | 255.831 | −0.402 | 1.00 | 50.00 | AAAA | C |
| ATOM | 3036 | C | TYR | A | 322 | 111.320 | 255.251 | 0.567 | 1.00 | 50.00 | AAAA | C |
| ATOM | 3037 | O | TYR | A | 322 | 110.738 | 254.195 | 0.352 | 1.00 | 50.00 | AAAA | O |
| ATOM | 3038 | CB | TYR | A | 322 | 111.723 | 256.991 | −1.211 | 1.00 | 50.00 | AAAA | C |
| ATOM | 3039 | CG | TYR | A | 322 | 110.561 | 256.525 | −2.064 | 1.00 | 50.00 | AAAA | C |
| ATOM | 3040 | CD1 | TYR | A | 322 | 109.253 | 256.578 | −1.535 | 1.00 | 50.00 | AAAA | C |
| ATOM | 3041 | CD2 | TYR | A | 322 | 110.816 | 256.042 | −3.363 | 1.00 | 50.00 | AAAA | C |
| ATOM | 3042 | CE1 | TYR | A | 322 | 108.181 | 256.110 | −2.315 | 1.00 | 50.00 | AAAA | C |
| ATOM | 3043 | CE2 | TYR | A | 322 | 109.747 | 255.576 | −4.142 | 1.00 | 50.00 | AAAA | C |
| ATOM | 3044 | CZ | TYR | A | 322 | 108.443 | 255.611 | −3.606 | 1.00 | 50.00 | AAAA | C |
| ATOM | 3045 | OH | TYR | A | 322 | 107.392 | 255.144 | −4.373 | 1.00 | 50.00 | AAAA | O |
| ATOM | 3046 | H | TYR | A | 322 | 112.488 | 254.577 | −2.143 | 0.00 | 0.00 | AAAA | H |
| ATOM | 3047 | HH | TYR | A | 322 | 107.704 | 254.872 | −5.227 | 0.00 | 0.00 | AAAA | H |
| ATOM | 3048 | N | VAL | A | 323 | 111.127 | 256.021 | 1.648 | 1.00 | 50.00 | AAAA | N |
| ATOM | 3049 | CA | VAL | A | 323 | 110.107 | 255.703 | 2.641 | 1.00 | 50.00 | AAAA | C |
| ATOM | 3050 | C | VAL | A | 323 | 109.354 | 256.968 | 3.028 | 1.00 | 50.00 | AAAA | C |
| ATOM | 3051 | O | VAL | A | 323 | 109.845 | 257.874 | 3.689 | 1.00 | 50.00 | AAAA | O |
| ATOM | 3052 | CB | VAL | A | 323 | 110.710 | 254.970 | 3.855 | 1.00 | 50.00 | AAAA | C |
| ATOM | 3053 | CG1 | VAL | A | 323 | 111.012 | 253.504 | 3.544 | 1.00 | 50.00 | AAAA | C |
| ATOM | 3054 | CG2 | VAL | A | 323 | 111.988 | 255.629 | 4.344 | 1.00 | 50.00 | AAAA | C |
| ATOM | 3055 | H | VAL | A | 323 | 111.656 | 256.859 | 1.766 | 0.00 | 0.00 | AAAA | H |
| ATOM | 3056 | N | ASN | A | 324 | 108.118 | 257.003 | 2.514 | 1.00 | 99.99 | AAAA | N |
| ATOM | 3057 | CA | ASN | A | 324 | 107.272 | 258.188 | 2.675 | 1.00 | 99.99 | AAAA | C |
| ATOM | 3058 | C | ASN | A | 324 | 106.695 | 258.396 | 4.082 | 1.00 | 99.99 | AAAA | C |
| ATOM | 3059 | O | ASN | A | 324 | 106.637 | 257.484 | 4.897 | 1.00 | 99.99 | AAAA | O |
| ATOM | 3060 | CB | ASN | A | 324 | 106.179 | 258.149 | 1.593 | 1.00 | 99.99 | AAAA | C |
| ATOM | 3061 | CG | ASN | A | 324 | 105.317 | 256.913 | 1.779 | 1.00 | 99.99 | AAAA | C |
| ATOM | 3062 | ND2 | ASN | A | 324 | 104.671 | 256.486 | 0.685 | 1.00 | 99.99 | AAAA | N |
| ATOM | 3063 | OD1 | ASN | A | 324 | 105.222 | 256.378 | 2.869 | 1.00 | 99.99 | AAAA | O |
| ATOM | 3064 | H | ASN | A | 324 | 107.765 | 256.194 | 2.041 | 0.00 | 0.00 | AAAA | H |
| ATOM | 3065 | 1HD2 | ASN | A | 324 | 104.074 | 255.689 | 0.793 | 0.00 | 0.00 | AAAA | H |
| ATOM | 3066 | 2HD2 | ASN | A | 324 | 104.733 | 256.911 | −0.218 | 0.00 | 0.00 | AAAA | H |
| ATOM | 3067 | N | LYS | A | 325 | 106.261 | 259.650 | 4.325 | 1.00 | 99.99 | AAAA | N |
| ATOM | 3068 | CA | LYS | A | 325 | 105.631 | 259.963 | 5.617 | 1.00 | 99.99 | AAAA | C |
| ATOM | 3069 | C | LYS | A | 325 | 104.142 | 259.759 | 5.654 | 1.00 | 99.99 | AAAA | C |
| ATOM | 3070 | O | LYS | A | 325 | 103.537 | 259.597 | 6.707 | 1.00 | 99.99 | AAAA | O |
| ATOM | 3071 | CB | LYS | A | 325 | 105.981 | 261.370 | 6.103 | 1.00 | 99.99 | AAAA | C |
| ATOM | 3072 | CG | LYS | A | 325 | 105.616 | 262.472 | 5.120 | 1.00 | 99.99 | AAAA | C |
| ATOM | 3073 | CD | LYS | A | 325 | 106.031 | 263.847 | 5.632 | 1.00 | 99.99 | AAAA | C |
| ATOM | 3074 | CE | LYS | A | 325 | 105.489 | 264.948 | 4.728 | 1.00 | 99.99 | AAAA | C |
| ATOM | 3075 | NZ | LYS | A | 325 | 104.019 | 264.897 | 4.746 | 1.00 | 99.99 | AAAA | N |
| ATOM | 3076 | H | LYS | A | 325 | 106.349 | 260.350 | 3.618 | 0.00 | 0.00 | AAAA | H |
| ATOM | 3077 | 1HZ | LYS | A | 325 | 103.637 | 265.619 | 4.104 | 0.00 | 0.00 | AAAA | H |
| ATOM | 3078 | 2HZ | LYS | A | 325 | 103.687 | 265.077 | 5.715 | 0.00 | 0.00 | AAAA | H |
| ATOM | 3079 | 3HZ | LYS | A | 325 | 103.694 | 263.956 | 4.444 | 0.00 | 0.00 | AAAA | H |
| ATOM | 3080 | N | GLN | A | 326 | 103.591 | 259.734 | 4.426 | 1.00 | 99.99 | AAAA | N |
| ATOM | 3081 | CA | GLN | A | 326 | 102.171 | 259.421 | 4.287 | 1.00 | 99.99 | AAAA | C |
| ATOM | 3082 | C | GLN | A | 326 | 101.810 | 258.031 | 4.809 | 1.00 | 99.99 | AAAA | C |
| ATOM | 3083 | O | GLN | A | 326 | 100.722 | 257.809 | 5.319 | 1.00 | 99.99 | AAAA | O |
| ATOM | 3084 | CB | GLN | A | 326 | 101.741 | 259.611 | 2.824 | 1.00 | 99.99 | AAAA | C |
| ATOM | 3085 | CG | GLN | A | 326 | 102.415 | 258.601 | 1.894 | 1.00 | 99.99 | AAAA | C |
| ATOM | 3086 | CD | GLN | A | 326 | 102.045 | 258.784 | 0.443 | 1.00 | 99.99 | AAAA | C |
| ATOM | 3087 | NE2 | GLN | A | 326 | 101.526 | 257.675 | −0.110 | 1.00 | 99.99 | AAAA | N |
| ATOM | 3088 | OE1 | GLN | A | 326 | 102.233 | 259.835 | −0.154 | 1.00 | 99.99 | AAAA | O |
| ATOM | 3089 | H | GLN | A | 326 | 104.153 | 259.930 | 3.626 | 0.00 | 0.00 | AAAA | H |
| ATOM | 3090 | 1HE2 | GLN | A | 326 | 101.274 | 257.667 | −1.077 | 0.00 | 0.00 | AAAA | H |
| ATOM | 3091 | 2HE2 | GLN | A | 326 | 101.394 | 256.848 | 0.438 | 0.00 | 0.00 | AAAA | H |
| ATOM | 3092 | N | GLU | A | 327 | 102.797 | 257.113 | 4.675 | 1.00 | 99.99 | AAAA | N |
| ATOM | 3093 | CA | GLU | A | 327 | 102.601 | 255.777 | 5.240 | 1.00 | 99.99 | AAAA | C |
| ATOM | 3094 | C | GLU | A | 327 | 103.008 | 255.608 | 6.682 | 1.00 | 99.99 | AAAA | C |
| ATOM | 3095 | O | GLU | A | 327 | 102.680 | 254.599 | 7.291 | 1.00 | 99.99 | AAAA | O |
| ATOM | 3096 | CB | GLU | A | 327 | 103.326 | 254.682 | 4.461 | 1.00 | 99.99 | AAAA | C |
| ATOM | 3097 | CG | GLU | A | 327 | 102.841 | 254.490 | 3.022 | 1.00 | 99.99 | AAAA | C |
| ATOM | 3098 | CD | GLU | A | 327 | 101.399 | 254.021 | 2.938 | 1.00 | 99.99 | AAAA | C |
| ATOM | 3099 | OE1 | GLU | A | 327 | 100.839 | 253.547 | 3.926 | 1.00 | 99.99 | AAAA | O |
| ATOM | 3100 | OE2 | GLU | A | 327 | 100.831 | 254.144 | 1.854 | 1.00 | 99.99 | AAAA | O |
| ATOM | 3101 | H | GLU | A | 327 | 103.663 | 257.362 | 4.242 | 0.00 | 0.00 | AAAA | H |
| ATOM | 3102 | N | GLY | A | 328 | 103.738 | 256.631 | 7.188 | 1.00 | 50.00 | AAAA | N |
| ATOM | 3103 | CA | GLY | A | 328 | 104.182 | 256.683 | 8.586 | 1.00 | 50.00 | AAAA | C |
| ATOM | 3104 | C | GLY | A | 328 | 103.332 | 255.931 | 9.595 | 1.00 | 50.00 | AAAA | C |
| ATOM | 3105 | O | GLY | A | 328 | 102.109 | 255.952 | 9.551 | 1.00 | 50.00 | AAAA | O |
| ATOM | 3106 | H | GLY | A | 328 | 104.048 | 257.342 | 6.560 | 0.00 | 0.00 | AAAA | H |
| ATOM | 3107 | N | LYS | A | 329 | 104.071 | 255.235 | 10.480 | 1.00 | 50.00 | AAAA | N |
| ATOM | 3108 | CA | LYS | A | 329 | 103.441 | 254.299 | 11.410 | 1.00 | 50.00 | AAAA | C |
| ATOM | 3109 | C | LYS | A | 329 | 102.369 | 254.930 | 12.277 | 1.00 | 50.00 | AAAA | C |
| ATOM | 3110 | O | LYS | A | 329 | 102.160 | 256.136 | 12.323 | 1.00 | 50.00 | AAAA | O |
| ATOM | 3111 | CB | LYS | A | 329 | 104.533 | 253.622 | 12.269 | 1.00 | 50.00 | AAAA | C |
| ATOM | 3112 | CG | LYS | A | 329 | 104.215 | 252.286 | 12.976 | 1.00 | 50.00 | AAAA | C |
| ATOM | 3113 | CD | LYS | A | 329 | 103.493 | 251.297 | 12.052 | 1.00 | 50.00 | AAAA | C |
| ATOM | 3114 | CE | LYS | A | 329 | 103.101 | 249.974 | 12.688 | 1.00 | 50.00 | AAAA | C |
| ATOM | 3115 | NZ | LYS | A | 329 | 101.917 | 250.214 | 13.517 | 1.00 | 50.00 | AAAA | N |
| ATOM | 3116 | H | LYS | A | 329 | 105.062 | 255.333 | 10.468 | 0.00 | 0.00 | AAAA | H |
| ATOM | 3117 | 1HZ | LYS | A | 329 | 101.642 | 249.333 | 13.999 | 0.00 | 0.00 | AAAA | H |
| ATOM | 3118 | 2HZ | LYS | A | 329 | 101.139 | 250.543 | 12.909 | 0.00 | 0.00 | AAAA | H |
| ATOM | 3119 | 3HZ | LYS | A | 329 | 102.148 | 250.947 | 14.215 | 0.00 | 0.00 | AAAA | H |
| ATOM | 3120 | N | SER | A | 330 | 101.721 | 254.016 | 12.987 | 1.00 | 50.00 | AAAA | N |
| ATOM | 3121 | CA | SER | A | 330 | 100.923 | 254.414 | 14.109 | 1.00 | 50.00 | AAAA | C |
| ATOM | 3122 | C | SER | A | 330 | 101.609 | 253.713 | 15.271 | 1.00 | 50.00 | AAAA | C |
| ATOM | 3123 | O | SER | A | 330 | 101.620 | 252.492 | 15.342 | 1.00 | 50.00 | AAAA | O |
| ATOM | 3124 | CB | SER | A | 330 | 99.526 | 253.918 | 13.740 | 1.00 | 50.00 | AAAA | C |
| ATOM | 3125 | OG | SER | A | 330 | 98.538 | 254.407 | 14.642 | 1.00 | 50.00 | AAAA | O |
| ATOM | 3126 | H | SER | A | 330 | 101.874 | 253.039 | 12.856 | 0.00 | 0.00 | AAAA | H |
| ATOM | 3127 | HG | SER | A | 330 | 97.758 | 253.884 | 14.495 | 0.00 | 0.00 | AAAA | H |
| ATOM | 3128 | N | LEU | A | 331 | 102.248 | 254.532 | 16.133 | 1.00 | 50.00 | AAAA | N |
| ATOM | 3129 | CA | LEU | A | 331 | 102.900 | 254.004 | 17.337 | 1.00 | 50.00 | AAAA | C |
| ATOM | 3130 | C | LEU | A | 331 | 101.941 | 253.225 | 18.209 | 1.00 | 50.00 | AAAA | C |
| ATOM | 3131 | O | LEU | A | 331 | 100.943 | 253.758 | 18.670 | 1.00 | 50.00 | AAAA | O |
| ATOM | 3132 | CB | LEU | A | 331 | 103.531 | 255.154 | 18.148 | 1.00 | 50.00 | AAAA | C |
| ATOM | 3133 | CG | LEU | A | 331 | 104.229 | 254.828 | 19.489 | 1.00 | 50.00 | AAAA | C |
| ATOM | 3134 | CD1 | LEU | A | 331 | 105.306 | 255.868 | 19.794 | 1.00 | 50.00 | AAAA | C |
| ATOM | 3135 | CD2 | LEU | A | 331 | 103.300 | 254.707 | 20.706 | 1.00 | 50.00 | AAAA | C |
| ATOM | 3136 | H | LEU | A | 331 | 102.314 | 255.503 | 15.909 | 0.00 | 0.00 | AAAA | H |
| ATOM | 3137 | N | TYR | A | 332 | 102.307 | 251.954 | 18.440 | 1.00 | 50.00 | AAAA | N |
| ATOM | 3138 | CA | TYR | A | 332 | 101.598 | 251.250 | 19.505 | 1.00 | 50.00 | AAAA | C |
| ATOM | 3139 | C | TYR | A | 332 | 102.503 | 250.279 | 20.254 | 1.00 | 50.00 | AAAA | C |
| ATOM | 3140 | O | TYR | A | 332 | 102.104 | 249.218 | 20.722 | 1.00 | 50.00 | AAAA | O |
| ATOM | 3141 | CB | TYR | A | 332 | 100.320 | 250.535 | 19.047 | 1.00 | 50.00 | AAAA | C |
| ATOM | 3142 | CG | TYR | A | 332 | 99.375 | 251.335 | 18.164 | 1.00 | 50.00 | AAAA | C |
| ATOM | 3143 | CD1 | TYR | A | 332 | 98.268 | 252.009 | 18.718 | 1.00 | 50.00 | AAAA | C |
| ATOM | 3144 | CD2 | TYR | A | 332 | 99.573 | 251.309 | 16.775 | 1.00 | 50.00 | AAAA | C |
| ATOM | 3145 | CE1 | TYR | A | 332 | 97.267 | 252.501 | 17.855 | 1.00 | 50.00 | AAAA | C |
| ATOM | 3146 | CE2 | TYR | A | 332 | 98.551 | 251.727 | 15.914 | 1.00 | 50.00 | AAAA | C |
| ATOM | 3147 | CZ | TYR | A | 332 | 97.390 | 252.293 | 16.464 | 1.00 | 50.00 | AAAA | C |
| ATOM | 3148 | OH | TYR | A | 332 | 96.356 | 252.650 | 15.614 | 1.00 | 50.00 | AAAA | O |
| ATOM | 3149 | H | TYR | A | 332 | 103.069 | 251.514 | 17.962 | 0.00 | 0.00 | AAAA | H |
| ATOM | 3150 | HH | TYR | A | 332 | 96.410 | 252.130 | 14.817 | 0.00 | 0.00 | AAAA | H |
| ATOM | 3151 | N | VAL | A | 333 | 103.765 | 250.732 | 20.377 | 1.00 | 50.00 | AAAA | N |
| ATOM | 3152 | CA | VAL | A | 333 | 104.740 | 250.103 | 21.275 | 1.00 | 50.00 | AAAA | C |
| ATOM | 3153 | C | VAL | A | 333 | 104.569 | 250.522 | 22.745 | 1.00 | 50.00 | AAAA | C |
| ATOM | 3154 | O | VAL | A | 333 | 105.507 | 250.781 | 23.488 | 1.00 | 50.00 | AAAA | O |
| ATOM | 3155 | CB | VAL | A | 333 | 106.159 | 250.369 | 20.720 | 1.00 | 50.00 | AAAA | C |
| ATOM | 3156 | CG1 | VAL | A | 333 | 106.499 | 251.864 | 20.676 | 1.00 | 50.00 | AAAA | C |
| ATOM | 3157 | CG2 | VAL | A | 333 | 107.247 | 249.503 | 21.369 | 1.00 | 50.00 | AAAA | C |
| ATOM | 3158 | H | VAL | A | 333 | 104.024 | 251.562 | 19.888 | 0.00 | 0.00 | AAAA | H |
| ATOM | 3159 | N | LYS | A | 334 | 103.277 | 250.584 | 23.129 | 1.00 | 50.00 | AAAA | N |
| ATOM | 3160 | CA | LYS | A | 334 | 102.886 | 251.057 | 24.458 | 1.00 | 50.00 | AAAA | C |
| ATOM | 3161 | C | LYS | A | 334 | 103.442 | 250.210 | 25.589 | 1.00 | 50.00 | AAAA | C |
| ATOM | 3162 | O | LYS | A | 334 | 103.893 | 250.700 | 26.615 | 1.00 | 50.00 | AAAA | O |
| ATOM | 3163 | CB | LYS | A | 334 | 101.357 | 251.099 | 24.534 | 1.00 | 50.00 | AAAA | C |
| ATOM | 3164 | CG | LYS | A | 334 | 100.717 | 252.075 | 23.541 | 1.00 | 50.00 | AAAA | C |
| ATOM | 3165 | CD | LYS | A | 334 | 100.991 | 253.538 | 23.894 | 1.00 | 50.00 | AAAA | C |
| ATOM | 3166 | CE | LYS | A | 334 | 100.387 | 253.913 | 25.248 | 1.00 | 50.00 | AAAA | C |
| ATOM | 3167 | NZ | LYS | A | 334 | 100.696 | 255.314 | 25.568 | 1.00 | 50.00 | AAAA | N |
| ATOM | 3168 | H | LYS | A | 334 | 102.561 | 250.301 | 22.494 | 0.00 | 0.00 | AAAA | H |
| ATOM | 3169 | 1HZ | LYS | A | 334 | 100.278 | 255.561 | 26.488 | 0.00 | 0.00 | AAAA | H |
| ATOM | 3170 | 2HZ | LYS | A | 334 | 100.302 | 255.931 | 24.828 | 0.00 | 0.00 | AAAA | H |
| ATOM | 3171 | 3HZ | LYS | A | 334 | 101.727 | 255.440 | 25.610 | 0.00 | 0.00 | AAAA | H |
| ATOM | 3172 | N | GLY | A | 335 | 103.400 | 248.891 | 25.327 | 1.00 | 50.00 | AAAA | N |
| ATOM | 3173 | CA | GLY | A | 335 | 103.985 | 247.985 | 26.309 | 1.00 | 50.00 | AAAA | C |
| ATOM | 3174 | C | GLY | A | 335 | 105.345 | 247.479 | 25.878 | 1.00 | 50.00 | AAAA | C |
| ATOM | 3175 | O | GLY | A | 335 | 105.578 | 247.181 | 24.712 | 1.00 | 50.00 | AAAA | O |
| ATOM | 3176 | H | GLY | A | 335 | 103.068 | 248.562 | 24.443 | 0.00 | 0.00 | AAAA | H |
| ATOM | 3177 | N | GLU | A | 336 | 106.224 | 247.388 | 26.891 | 1.00 | 50.00 | AAAA | N |
| ATOM | 3178 | CA | GLU | A | 336 | 107.545 | 246.794 | 26.675 | 1.00 | 50.00 | AAAA | C |
| ATOM | 3179 | C | GLU | A | 336 | 107.495 | 245.284 | 26.443 | 1.00 | 50.00 | AAAA | C |
| ATOM | 3180 | O | GLU | A | 336 | 106.662 | 244.578 | 27.000 | 1.00 | 50.00 | AAAA | O |
| ATOM | 3181 | CB | GLU | A | 336 | 108.469 | 247.148 | 27.847 | 1.00 | 50.00 | AAAA | C |
| ATOM | 3182 | CG | GLU | A | 336 | 108.009 | 246.578 | 29.193 | 1.00 | 50.00 | AAAA | C |
| ATOM | 3183 | CD | GLU | A | 336 | 109.028 | 246.901 | 30.266 | 1.00 | 50.00 | AAAA | C |
| ATOM | 3184 | OE1 | GLU | A | 336 | 109.174 | 248.076 | 30.603 | 1.00 | 50.00 | AAAA | O |
| ATOM | 3185 | OE2 | GLU | A | 336 | 109.665 | 245.973 | 30.765 | 1.00 | 50.00 | AAAA | O |
| ATOM | 3186 | H | GLU | A | 336 | 105.952 | 247.693 | 27.802 | 0.00 | 0.00 | AAAA | H |
| ATOM | 3187 | N | PRO | A | 337 | 108.413 | 244.803 | 25.566 | 1.00 | 50.00 | AAAA | N |
| ATOM | 3188 | CA | PRO | A | 337 | 108.478 | 243.358 | 25.329 | 1.00 | 50.00 | AAAA | C |
| ATOM | 3189 | C | PRO | A | 337 | 109.174 | 242.575 | 26.438 | 1.00 | 50.00 | AAAA | C |
| ATOM | 3190 | O | PRO | A | 337 | 110.311 | 242.830 | 26.819 | 1.00 | 50.00 | AAAA | O |
| ATOM | 3191 | CB | PRO | A | 337 | 109.232 | 243.285 | 23.999 | 1.00 | 50.00 | AAAA | C |
| ATOM | 3192 | CG | PRO | A | 337 | 110.164 | 244.495 | 24.012 | 1.00 | 50.00 | AAAA | C |
| ATOM | 3193 | CD | PRO | A | 337 | 109.346 | 245.561 | 24.732 | 1.00 | 50.00 | AAAA | C |
| ATOM | 3194 | N | ILE | A | 338 | 108.422 | 241.571 | 26.915 | 1.00 | 50.00 | AAAA | N |
| ATOM | 3195 | CA | ILE | A | 338 | 109.035 | 240.676 | 27.893 | 1.00 | 50.00 | AAAA | C |
| ATOM | 3196 | C | ILE | A | 338 | 109.356 | 239.300 | 27.325 | 1.00 | 50.00 | AAAA | C |
| ATOM | 3197 | O | ILE | A | 338 | 108.495 | 238.523 | 26.921 | 1.00 | 50.00 | AAAA | O |
| ATOM | 3198 | CB | ILE | A | 338 | 108.200 | 240.586 | 29.187 | 1.00 | 50.00 | AAAA | C |
| ATOM | 3199 | CG1 | ILE | A | 338 | 106.827 | 239.950 | 28.973 | 1.00 | 50.00 | AAAA | C |
| ATOM | 3200 | CG2 | ILE | A | 338 | 108.045 | 241.982 | 29.800 | 1.00 | 50.00 | AAAA | C |
| ATOM | 3201 | CD1 | ILE | A | 338 | 106.130 | 239.532 | 30.269 | 1.00 | 50.00 | AAAA | C |
| ATOM | 3202 | H | ILE | A | 338 | 107.501 | 241.416 | 26.560 | 0.00 | 0.00 | AAAA | H |
| ATOM | 3203 | N | ILE | A | 339 | 110.671 | 239.032 | 27.280 | 1.00 | 50.00 | AAAA | N |
| ATOM | 3204 | CA | ILE | A | 339 | 111.057 | 237.757 | 26.677 | 1.00 | 50.00 | AAAA | C |
| ATOM | 3205 | C | ILE | A | 339 | 112.087 | 237.008 | 27.504 | 1.00 | 50.00 | AAAA | C |
| ATOM | 3206 | O | ILE | A | 339 | 113.083 | 237.562 | 27.952 | 1.00 | 50.00 | AAAA | O |
| ATOM | 3207 | CB | ILE | A | 339 | 111.534 | 237.939 | 25.220 | 1.00 | 50.00 | AAAA | C |
| ATOM | 3208 | CG1 | ILE | A | 339 | 112.700 | 238.923 | 25.098 | 1.00 | 50.00 | AAAA | C |
| ATOM | 3209 | CG2 | ILE | A | 339 | 110.384 | 238.379 | 24.302 | 1.00 | 50.00 | AAAA | C |
| ATOM | 3210 | CD1 | ILE | A | 339 | 113.127 | 239.073 | 23.641 | 1.00 | 50.00 | AAAA | C |
| ATOM | 3211 | H | ILE | A | 339 | 111.348 | 239.685 | 27.615 | 0.00 | 0.00 | AAAA | H |
| ATOM | 3212 | N | ASN | A | 340 | 111.800 | 235.708 | 27.687 | 1.00 | 50.00 | AAAA | N |
| ATOM | 3213 | CA | ASN | A | 340 | 112.789 | 234.905 | 28.403 | 1.00 | 50.00 | AAAA | C |
| ATOM | 3214 | C | ASN | A | 340 | 113.529 | 233.976 | 27.464 | 1.00 | 50.00 | AAAA | C |
| ATOM | 3215 | O | ASN | A | 340 | 112.979 | 233.483 | 26.489 | 1.00 | 50.00 | AAAA | O |
| ATOM | 3216 | CB | ASN | A | 340 | 112.143 | 234.105 | 29.541 | 1.00 | 50.00 | AAAA | C |
| ATOM | 3217 | CG | ASN | A | 340 | 111.630 | 235.029 | 30.632 | 1.00 | 50.00 | AAAA | C |
| ATOM | 3218 | ND2 | ASN | A | 340 | 110.589 | 234.520 | 31.313 | 1.00 | 50.00 | AAAA | N |
| ATOM | 3219 | OD1 | ASN | A | 340 | 112.134 | 236.121 | 30.861 | 1.00 | 50.00 | AAAA | O |
| ATOM | 3220 | H | ASN | A | 340 | 110.958 | 235.286 | 27.348 | 0.00 | 0.00 | AAAA | H |
| ATOM | 3221 | 1HD2 | ASN | A | 340 | 110.166 | 235.049 | 32.048 | 0.00 | 0.00 | AAAA | H |
| ATOM | 3222 | 2HD2 | ASN | A | 340 | 110.213 | 233.617 | 31.106 | 0.00 | 0.00 | AAAA | H |
| ATOM | 3223 | N | PHE | A | 341 | 114.810 | 233.745 | 27.812 | 1.00 | 50.00 | AAAA | N |
| ATOM | 3224 | CA | PHE | A | 341 | 115.653 | 232.856 | 27.001 | 1.00 | 50.00 | AAAA | C |
| ATOM | 3225 | C | PHE | A | 341 | 115.266 | 231.377 | 26.955 | 1.00 | 50.00 | AAAA | C |
| ATOM | 3226 | O | PHE | A | 341 | 115.795 | 230.595 | 26.177 | 1.00 | 50.00 | AAAA | O |
| ATOM | 3227 | CB | PHE | A | 341 | 117.131 | 233.022 | 27.375 | 1.00 | 50.00 | AAAA | C |
| ATOM | 3228 | CG | PHE | A | 341 | 117.400 | 232.479 | 28.760 | 1.00 | 50.00 | AAAA | C |
| ATOM | 3229 | CD1 | PHE | A | 341 | 117.758 | 231.120 | 28.913 | 1.00 | 50.00 | AAAA | C |
| ATOM | 3230 | CD2 | PHE | A | 341 | 117.285 | 233.336 | 29.875 | 1.00 | 50.00 | AAAA | C |
| ATOM | 3231 | CE1 | PHE | A | 341 | 118.007 | 230.612 | 30.200 | 1.00 | 50.00 | AAAA | C |
| ATOM | 3232 | CE2 | PHE | A | 341 | 117.534 | 232.830 | 31.163 | 1.00 | 50.00 | AAAA | C |
| ATOM | 3233 | CZ | PHE | A | 341 | 117.900 | 231.477 | 31.311 | 1.00 | 50.00 | AAAA | C |
| ATOM | 3234 | H | PHE | A | 341 | 115.193 | 234.232 | 28.595 | 0.00 | 0.00 | AAAA | H |
| ATOM | 3235 | N | TYR | A | 342 | 114.293 | 231.032 | 27.815 | 1.00 | 50.00 | AAAA | N |
| ATOM | 3236 | CA | TYR | A | 342 | 113.711 | 229.692 | 27.727 | 1.00 | 50.00 | AAAA | C |
| ATOM | 3237 | C | TYR | A | 342 | 112.796 | 229.481 | 26.519 | 1.00 | 50.00 | AAAA | C |
| ATOM | 3238 | O | TYR | A | 342 | 112.557 | 228.372 | 26.067 | 1.00 | 50.00 | AAAA | O |
| ATOM | 3239 | CB | TYR | A | 342 | 112.989 | 229.363 | 29.037 | 1.00 | 50.00 | AAAA | C |
| ATOM | 3240 | CG | TYR | A | 342 | 113.947 | 229.356 | 30.213 | 1.00 | 50.00 | AAAA | C |
| ATOM | 3241 | CD1 | TYR | A | 342 | 114.743 | 228.215 | 30.443 | 1.00 | 50.00 | AAAA | C |
| ATOM | 3242 | CD2 | TYR | A | 342 | 114.004 | 230.484 | 31.059 | 1.00 | 50.00 | AAAA | C |
| ATOM | 3243 | CE1 | TYR | A | 342 | 115.600 | 228.192 | 31.556 | 1.00 | 50.00 | AAAA | C |
| ATOM | 3244 | CE2 | TYR | A | 342 | 114.857 | 230.459 | 32.174 | 1.00 | 50.00 | AAAA | C |
| ATOM | 3245 | CZ | TYR | A | 342 | 115.647 | 229.313 | 32.408 | 1.00 | 50.00 | AAAA | C |
| ATOM | 3246 | OH | TYR | A | 342 | 116.498 | 229.289 | 33.496 | 1.00 | 50.00 | AAAA | O |
| ATOM | 3247 | H | TYR | A | 342 | 113.958 | 231.700 | 28.477 | 0.00 | 0.00 | AAAA | H |
| ATOM | 3248 | HH | TYR | A | 342 | 116.548 | 230.148 | 33.897 | 0.00 | 0.00 | AAAA | H |
| ATOM | 3249 | N | ASP | A | 343 | 112.322 | 230.622 | 25.986 | 1.00 | 50.00 | AAAA | N |
| ATOM | 3250 | CA | ASP | A | 343 | 111.589 | 230.594 | 24.713 | 1.00 | 50.00 | AAAA | C |
| ATOM | 3251 | C | ASP | A | 343 | 112.404 | 230.225 | 23.457 | 1.00 | 50.00 | AAAA | C |
| ATOM | 3252 | O | ASP | A | 343 | 111.944 | 229.429 | 22.645 | 1.00 | 50.00 | AAAA | O |
| ATOM | 3253 | CB | ASP | A | 343 | 110.755 | 231.878 | 24.526 | 1.00 | 50.00 | AAAA | C |
| ATOM | 3254 | CG | ASP | A | 343 | 109.856 | 232.134 | 25.726 | 1.00 | 50.00 | AAAA | C |
| ATOM | 3255 | OD1 | ASP | A | 343 | 109.010 | 231.291 | 26.026 | 1.00 | 50.00 | AAAA | O |
| ATOM | 3256 | OD2 | ASP | A | 343 | 110.011 | 233.165 | 26.378 | 1.00 | 50.00 | AAAA | O |
| ATOM | 3257 | H | ASP | A | 343 | 112.535 | 231.491 | 26.430 | 0.00 | 0.00 | AAAA | H |
| ATOM | 3258 | N | PRO | A | 344 | 113.647 | 230.791 | 23.305 | 1.00 | 50.00 | AAAA | N |
| ATOM | 3259 | CA | PRO | A | 344 | 114.641 | 230.227 | 22.374 | 1.00 | 50.00 | AAAA | C |
| ATOM | 3260 | C | PRO | A | 344 | 114.944 | 228.744 | 22.504 | 1.00 | 50.00 | AAAA | C |
| ATOM | 3261 | O | PRO | A | 344 | 115.780 | 228.315 | 23.291 | 1.00 | 50.00 | AAAA | O |
| ATOM | 3262 | CB | PRO | A | 344 | 115.911 | 231.041 | 22.636 | 1.00 | 50.00 | AAAA | C |
| ATOM | 3263 | CG | PRO | A | 344 | 115.445 | 232.373 | 23.195 | 1.00 | 50.00 | AAAA | C |
| ATOM | 3264 | CD | PRO | A | 344 | 114.153 | 232.018 | 23.918 | 1.00 | 50.00 | AAAA | C |
| ATOM | 3265 | N | LEU | A | 345 | 114.256 | 227.997 | 21.631 | 1.00 | 50.00 | AAAA | N |
| ATOM | 3266 | CA | LEU | A | 345 | 114.606 | 226.601 | 21.463 | 1.00 | 50.00 | AAAA | C |
| ATOM | 3267 | C | LEU | A | 345 | 114.432 | 226.144 | 20.034 | 1.00 | 50.00 | AAAA | C |
| ATOM | 3268 | O | LEU | A | 345 | 113.834 | 226.791 | 19.185 | 1.00 | 50.00 | AAAA | O |
| ATOM | 3269 | CB | LEU | A | 345 | 113.823 | 225.713 | 22.446 | 1.00 | 50.00 | AAAA | C |
| ATOM | 3270 | CG | LEU | A | 345 | 112.296 | 225.881 | 22.437 | 1.00 | 50.00 | AAAA | C |
| ATOM | 3271 | CD1 | LEU | A | 345 | 111.586 | 225.117 | 21.313 | 1.00 | 50.00 | AAAA | C |
| ATOM | 3272 | CD2 | LEU | A | 345 | 111.713 | 225.502 | 23.797 | 1.00 | 50.00 | AAAA | C |
| ATOM | 3273 | H | LEU | A | 345 | 113.516 | 228.384 | 21.084 | 0.00 | 0.00 | AAAA | H |
| ATOM | 3274 | N | VAL | A | 346 | 114.991 | 224.951 | 19.854 | 1.00 | 50.00 | AAAA | N |
| ATOM | 3275 | CA | VAL | A | 346 | 114.826 | 224.198 | 18.624 | 1.00 | 50.00 | AAAA | C |
| ATOM | 3276 | C | VAL | A | 346 | 113.578 | 223.325 | 18.648 | 1.00 | 50.00 | AAAA | C |
| ATOM | 3277 | O | VAL | A | 346 | 113.293 | 222.620 | 19.606 | 1.00 | 50.00 | AAAA | O |
| ATOM | 3278 | CB | VAL | A | 346 | 116.087 | 223.353 | 18.411 | 1.00 | 50.00 | AAAA | C |
| ATOM | 3279 | CG1 | VAL | A | 346 | 117.261 | 224.214 | 17.943 | 1.00 | 50.00 | AAAA | C |
| ATOM | 3280 | CG2 | VAL | A | 346 | 116.462 | 222.586 | 19.688 | 1.00 | 50.00 | AAAA | C |
| ATOM | 3281 | H | VAL | A | 346 | 115.461 | 224.546 | 20.633 | 0.00 | 0.00 | AAAA | H |
| ATOM | 3282 | N | PHE | A | 347 | 112.849 | 223.400 | 17.527 | 1.00 | 50.00 | AAAA | N |
| ATOM | 3283 | CA | PHE | A | 347 | 111.776 | 222.433 | 17.292 | 1.00 | 50.00 | AAAA | C |
| ATOM | 3284 | C | PHE | A | 347 | 112.001 | 221.243 | 16.332 | 1.00 | 50.00 | AAAA | C |
| ATOM | 3285 | O | PHE | A | 347 | 111.124 | 220.390 | 16.264 | 1.00 | 50.00 | AAAA | O |
| ATOM | 3286 | CB | PHE | A | 347 | 110.568 | 223.297 | 16.953 | 1.00 | 50.00 | AAAA | C |
| ATOM | 3287 | CG | PHE | A | 347 | 109.222 | 222.648 | 16.702 | 1.00 | 50.00 | AAAA | C |
| ATOM | 3288 | CD1 | PHE | A | 347 | 108.310 | 222.525 | 17.771 | 1.00 | 50.00 | AAAA | C |
| ATOM | 3289 | CD2 | PHE | A | 347 | 108.865 | 222.252 | 15.391 | 1.00 | 50.00 | AAAA | C |
| ATOM | 3290 | CE1 | PHE | A | 347 | 107.009 | 222.045 | 17.522 | 1.00 | 50.00 | AAAA | C |
| ATOM | 3291 | CE2 | PHE | A | 347 | 107.566 | 221.770 | 15.140 | 1.00 | 50.00 | AAAA | C |
| ATOM | 3292 | CZ | PHE | A | 347 | 106.645 | 221.688 | 16.205 | 1.00 | 50.00 | AAAA | C |
| ATOM | 3293 | H | PHE | A | 347 | 113.048 | 224.101 | 16.842 | 0.00 | 0.00 | AAAA | H |
| ATOM | 3294 | N | PRO | A | 348 | 113.151 | 221.135 | 15.582 | 1.00 | 50.00 | AAAA | N |
| ATOM | 3295 | CA | PRO | A | 348 | 113.363 | 219.877 | 14.846 | 1.00 | 50.00 | AAAA | C |
| ATOM | 3296 | C | PRO | A | 348 | 113.451 | 218.662 | 15.749 | 1.00 | 50.00 | AAAA | C |
| ATOM | 3297 | O | PRO | A | 348 | 114.000 | 218.725 | 16.839 | 1.00 | 50.00 | AAAA | O |
| ATOM | 3298 | CB | PRO | A | 348 | 114.696 | 220.084 | 14.117 | 1.00 | 50.00 | AAAA | C |
| ATOM | 3299 | CG | PRO | A | 348 | 114.885 | 221.590 | 14.036 | 1.00 | 50.00 | AAAA | C |
| ATOM | 3300 | CD | PRO | A | 348 | 114.256 | 222.061 | 15.335 | 1.00 | 50.00 | AAAA | C |
| ATOM | 3301 | N | SER | A | 349 | 112.901 | 217.555 | 15.213 | 1.00 | 50.00 | AAAA | N |
| ATOM | 3302 | CA | SER | A | 349 | 112.861 | 216.290 | 15.952 | 1.00 | 50.00 | AAAA | C |
| ATOM | 3303 | C | SER | A | 349 | 114.151 | 215.856 | 16.626 | 1.00 | 50.00 | AAAA | C |
| ATOM | 3304 | O | SER | A | 349 | 114.167 | 215.562 | 17.811 | 1.00 | 50.00 | AAAA | O |
| ATOM | 3305 | CB | SER | A | 349 | 112.382 | 215.160 | 15.044 | 1.00 | 50.00 | AAAA | C |
| ATOM | 3306 | OG | SER | A | 349 | 111.118 | 215.498 | 14.474 | 1.00 | 50.00 | AAAA | O |
| ATOM | 3307 | H | SER | A | 349 | 112.409 | 217.641 | 14.349 | 0.00 | 0.00 | AAAA | H |
| ATOM | 3308 | HG | SER | A | 349 | 110.858 | 214.758 | 13.940 | 0.00 | 0.00 | AAAA | H |
| ATOM | 3309 | N | ASP | A | 350 | 115.235 | 215.842 | 15.818 | 1.00 | 50.00 | AAAA | N |
| ATOM | 3310 | CA | ASP | A | 350 | 116.544 | 215.422 | 16.342 | 1.00 | 50.00 | AAAA | C |
| ATOM | 3311 | C | ASP | A | 350 | 116.977 | 216.192 | 17.580 | 1.00 | 50.00 | AAAA | C |
| ATOM | 3312 | O | ASP | A | 350 | 117.368 | 215.641 | 18.599 | 1.00 | 50.00 | AAAA | O |
| ATOM | 3313 | CB | ASP | A | 350 | 117.603 | 215.536 | 15.232 | 1.00 | 50.00 | AAAA | C |
| ATOM | 3314 | CG | ASP | A | 350 | 118.977 | 215.108 | 15.725 | 1.00 | 50.00 | AAAA | C |
| ATOM | 3315 | OD1 | ASP | A | 350 | 119.147 | 213.934 | 16.047 | 1.00 | 50.00 | AAAA | O |
| ATOM | 3316 | OD2 | ASP | A | 350 | 119.867 | 215.954 | 15.787 | 1.00 | 50.00 | AAAA | O |
| ATOM | 3317 | H | ASP | A | 350 | 115.130 | 216.090 | 14.856 | 0.00 | 0.00 | AAAA | H |
| ATOM | 3318 | N | GLU | A | 351 | 116.852 | 217.513 | 17.408 | 1.00 | 50.00 | AAAA | N |
| ATOM | 3319 | CA | GLU | A | 351 | 117.191 | 218.452 | 18.465 | 1.00 | 50.00 | AAAA | C |
| ATOM | 3320 | C | GLU | A | 351 | 116.333 | 218.331 | 19.719 | 1.00 | 50.00 | AAAA | C |
| ATOM | 3321 | O | GLU | A | 351 | 116.817 | 218.404 | 20.840 | 1.00 | 50.00 | AAAA | O |
| ATOM | 3322 | CB | GLU | A | 351 | 117.112 | 219.837 | 17.842 | 1.00 | 50.00 | AAAA | C |
| ATOM | 3323 | CG | GLU | A | 351 | 118.234 | 220.191 | 16.856 | 1.00 | 50.00 | AAAA | C |
| ATOM | 3324 | CD | GLU | A | 351 | 119.567 | 220.467 | 17.547 | 1.00 | 50.00 | AAAA | C |
| ATOM | 3325 | OE1 | GLU | A | 351 | 119.659 | 220.367 | 18.771 | 1.00 | 50.00 | AAAA | O |
| ATOM | 3326 | OE2 | GLU | A | 351 | 120.522 | 220.780 | 16.845 | 1.00 | 50.00 | AAAA | O |
| ATOM | 3327 | H | GLU | A | 351 | 116.483 | 217.858 | 16.547 | 0.00 | 0.00 | AAAA | H |
| ATOM | 3328 | N | PHE | A | 352 | 115.031 | 218.098 | 19.463 | 1.00 | 50.00 | AAAA | N |
| ATOM | 3329 | CA | PHE | A | 352 | 114.087 | 217.864 | 20.554 | 1.00 | 50.00 | AAAA | C |
| ATOM | 3330 | C | PHE | A | 352 | 114.395 | 216.600 | 21.346 | 1.00 | 50.00 | AAAA | C |
| ATOM | 3331 | O | PHE | A | 352 | 114.306 | 216.568 | 22.562 | 1.00 | 50.00 | AAAA | O |
| ATOM | 3332 | CB | PHE | A | 352 | 112.660 | 217.848 | 19.985 | 1.00 | 50.00 | AAAA | C |
| ATOM | 3333 | CG | PHE | A | 352 | 111.613 | 217.718 | 21.072 | 1.00 | 50.00 | AAAA | C |
| ATOM | 3334 | CD1 | PHE | A | 352 | 111.248 | 218.851 | 21.832 | 1.00 | 50.00 | AAAA | C |
| ATOM | 3335 | CD2 | PHE | A | 352 | 111.015 | 216.460 | 21.303 | 1.00 | 50.00 | AAAA | C |
| ATOM | 3336 | CE1 | PHE | A | 352 | 110.270 | 218.724 | 22.837 | 1.00 | 50.00 | AAAA | C |
| ATOM | 3337 | CE2 | PHE | A | 352 | 110.036 | 216.332 | 22.308 | 1.00 | 50.00 | AAAA | C |
| ATOM | 3338 | CZ | PHE | A | 352 | 109.674 | 217.465 | 23.066 | 1.00 | 50.00 | AAAA | C |
| ATOM | 3339 | H | PHE | A | 352 | 114.714 | 218.039 | 18.520 | 0.00 | 0.00 | AAAA | H |
| ATOM | 3340 | N | ASP | A | 353 | 114.787 | 215.560 | 20.584 | 1.00 | 50.00 | AAAA | N |
| ATOM | 3341 | CA | ASP | A | 353 | 115.166 | 214.282 | 21.190 | 1.00 | 50.00 | AAAA | C |
| ATOM | 3342 | C | ASP | A | 353 | 116.427 | 214.359 | 22.038 | 1.00 | 50.00 | AAAA | C |
| ATOM | 3343 | O | ASP | A | 353 | 116.560 | 213.690 | 23.053 | 1.00 | 50.00 | AAAA | O |
| ATOM | 3344 | CB | ASP | A | 353 | 115.270 | 213.200 | 20.106 | 1.00 | 50.00 | AAAA | C |
| ATOM | 3345 | CG | ASP | A | 353 | 115.581 | 211.836 | 20.701 | 1.00 | 50.00 | AAAA | C |
| ATOM | 3346 | OD1 | ASP | A | 353 | 114.716 | 211.267 | 21.363 | 1.00 | 50.00 | AAAA | O |
| ATOM | 3347 | OD2 | ASP | A | 353 | 116.690 | 211.351 | 20.485 | 1.00 | 50.00 | AAAA | O |
| ATOM | 3348 | H | ASP | A | 353 | 114.867 | 215.700 | 19.600 | 0.00 | 0.00 | AAAA | H |
| ATOM | 3349 | N | ALA | A | 354 | 117.340 | 215.237 | 21.588 | 1.00 | 50.00 | AAAA | N |
| ATOM | 3350 | CA | ALA | A | 354 | 118.512 | 215.485 | 22.425 | 1.00 | 50.00 | AAAA | C |
| ATOM | 3351 | C | ALA | A | 354 | 118.162 | 216.177 | 23.735 | 1.00 | 50.00 | AAAA | C |
| ATOM | 3352 | O | ALA | A | 354 | 118.606 | 215.786 | 24.808 | 1.00 | 50.00 | AAAA | O |
| ATOM | 3353 | CB | ALA | A | 354 | 119.545 | 216.311 | 21.658 | 1.00 | 50.00 | AAAA | C |
| ATOM | 3354 | H | ALA | A | 354 | 117.180 | 215.759 | 20.750 | 0.00 | 0.00 | AAAA | H |
| ATOM | 3355 | N | SER | A | 355 | 117.283 | 217.188 | 23.587 | 1.00 | 50.00 | AAAA | N |
| ATOM | 3356 | CA | SER | A | 355 | 116.762 | 217.890 | 24.759 | 1.00 | 50.00 | AAAA | C |
| ATOM | 3357 | C | SER | A | 355 | 115.946 | 217.029 | 25.721 | 1.00 | 50.00 | AAAA | C |
| ATOM | 3358 | O | SER | A | 355 | 115.963 | 217.221 | 26.929 | 1.00 | 50.00 | AAAA | O |
| ATOM | 3359 | CB | SER | A | 355 | 115.959 | 219.122 | 24.327 | 1.00 | 50.00 | AAAA | C |
| ATOM | 3360 | OG | SER | A | 355 | 116.760 | 219.970 | 23.495 | 1.00 | 50.00 | AAAA | O |
| ATOM | 3361 | H | SER | A | 355 | 117.005 | 217.477 | 22.673 | 0.00 | 0.00 | AAAA | H |
| ATOM | 3362 | HG | SER | A | 355 | 116.227 | 220.721 | 23.269 | 0.00 | 0.00 | AAAA | H |
| ATOM | 3363 | N | ILE | A | 356 | 115.241 | 216.035 | 25.150 | 1.00 | 50.00 | AAAA | N |
| ATOM | 3364 | CA | ILE | A | 356 | 114.518 | 215.158 | 26.073 | 1.00 | 50.00 | AAAA | C |
| ATOM | 3365 | C | ILE | A | 356 | 115.410 | 214.162 | 26.789 | 1.00 | 50.00 | AAAA | C |
| ATOM | 3366 | O | ILE | A | 356 | 115.142 | 213.765 | 27.913 | 1.00 | 50.00 | AAAA | O |
| ATOM | 3367 | CB | ILE | A | 356 | 113.319 | 214.444 | 25.431 | 1.00 | 50.00 | AAAA | C |
| ATOM | 3368 | CG1 | ILE | A | 356 | 113.723 | 213.415 | 24.371 | 1.00 | 50.00 | AAAA | C |
| ATOM | 3369 | CG2 | ILE | A | 356 | 112.366 | 215.495 | 24.865 | 1.00 | 50.00 | AAAA | C |
| ATOM | 3370 | CD1 | ILE | A | 356 | 112.554 | 212.685 | 23.712 | 1.00 | 50.00 | AAAA | C |
| ATOM | 3371 | H | ILE | A | 356 | 115.268 | 215.900 | 24.161 | 0.00 | 0.00 | AAAA | H |
| ATOM | 3372 | N | SER | A | 357 | 116.506 | 213.813 | 26.088 | 1.00 | 50.00 | AAAA | N |
| ATOM | 3373 | CA | SER | A | 357 | 117.493 | 212.922 | 26.687 | 1.00 | 50.00 | AAAA | C |
| ATOM | 3374 | C | SER | A | 357 | 118.291 | 213.590 | 27.797 | 1.00 | 50.00 | AAAA | C |
| ATOM | 3375 | O | SER | A | 357 | 118.674 | 212.962 | 28.775 | 1.00 | 50.00 | AAAA | O |
| ATOM | 3376 | CB | SER | A | 357 | 118.404 | 212.362 | 25.588 | 1.00 | 50.00 | AAAA | C |
| ATOM | 3377 | OG | SER | A | 357 | 119.202 | 211.288 | 26.096 | 1.00 | 50.00 | AAAA | O |
| ATOM | 3378 | H | SER | A | 357 | 116.665 | 214.182 | 25.173 | 0.00 | 0.00 | AAAA | H |
| ATOM | 3379 | HG | SER | A | 357 | 119.837 | 211.072 | 25.427 | 0.00 | 0.00 | AAAA | H |
| ATOM | 3380 | N | GLN | A | 358 | 118.487 | 214.913 | 27.612 | 1.00 | 50.00 | AAAA | N |
| ATOM | 3381 | CA | GLN | A | 358 | 119.165 | 215.684 | 28.654 | 1.00 | 50.00 | AAAA | C |
| ATOM | 3382 | C | GLN | A | 358 | 118.311 | 215.862 | 29.910 | 1.00 | 50.00 | AAAA | C |
| ATOM | 3383 | O | GLN | A | 358 | 118.789 | 215.765 | 31.032 | 1.00 | 50.00 | AAAA | O |
| ATOM | 3384 | CB | GLN | A | 358 | 119.691 | 217.021 | 28.088 | 1.00 | 50.00 | AAAA | C |
| ATOM | 3385 | CG | GLN | A | 358 | 118.624 | 218.118 | 28.025 | 1.00 | 50.00 | AAAA | C |
| ATOM | 3386 | CD | GLN | A | 358 | 119.012 | 219.338 | 27.232 | 1.00 | 50.00 | AAAA | C |
| ATOM | 3387 | NE2 | GLN | A | 358 | 118.476 | 220.453 | 27.754 | 1.00 | 50.00 | AAAA | N |
| ATOM | 3388 | OE1 | GLN | A | 358 | 119.696 | 219.285 | 26.217 | 1.00 | 50.00 | AAAA | O |
| ATOM | 3389 | H | GLN | A | 358 | 118.141 | 215.355 | 26.787 | 0.00 | 0.00 | AAAA | H |
| ATOM | 3390 | 1HE2 | GLN | A | 358 | 118.610 | 221.347 | 27.334 | 0.00 | 0.00 | AAAA | H |
| ATOM | 3391 | 2HE2 | GLN | A | 358 | 117.918 | 220.371 | 28.581 | 0.00 | 0.00 | AAAA | H |
| ATOM | 3392 | N | VAL | A | 359 | 117.000 | 216.086 | 29.654 | 1.00 | 50.00 | AAAA | N |
| ATOM | 3393 | CA | VAL | A | 359 | 116.038 | 216.220 | 30.748 | 1.00 | 50.00 | AAAA | C |
| ATOM | 3394 | C | VAL | A | 359 | 115.915 | 214.918 | 31.515 | 1.00 | 50.00 | AAAA | C |
| ATOM | 3395 | O | VAL | A | 359 | 115.951 | 214.889 | 32.735 | 1.00 | 50.00 | AAAA | O |
| ATOM | 3396 | CB | VAL | A | 359 | 114.676 | 216.702 | 30.206 | 1.00 | 50.00 | AAAA | C |
| ATOM | 3397 | CG1 | VAL | A | 359 | 113.528 | 216.590 | 31.219 | 1.00 | 50.00 | AAAA | C |
| ATOM | 3398 | CG2 | VAL | A | 359 | 114.796 | 218.141 | 29.700 | 1.00 | 50.00 | AAAA | C |
| ATOM | 3399 | H | VAL | A | 359 | 116.696 | 216.182 | 28.708 | 0.00 | 0.00 | AAAA | H |
| ATOM | 3400 | N | ASN | A | 360 | 115.822 | 213.837 | 30.720 | 1.00 | 50.00 | AAAA | N |
| ATOM | 3401 | CA | ASN | A | 360 | 115.743 | 212.493 | 31.291 | 1.00 | 50.00 | AAAA | C |
| ATOM | 3402 | C | ASN | A | 360 | 116.980 | 212.080 | 32.075 | 1.00 | 50.00 | AAAA | C |
| ATOM | 3403 | O | ASN | A | 360 | 116.910 | 211.385 | 33.078 | 1.00 | 50.00 | AAAA | O |
| ATOM | 3404 | CB | ASN | A | 360 | 115.405 | 211.477 | 30.193 | 1.00 | 50.00 | AAAA | C |
| ATOM | 3405 | CG | ASN | A | 360 | 115.152 | 210.098 | 30.777 | 1.00 | 50.00 | AAAA | C |
| ATOM | 3406 | ND2 | ASN | A | 360 | 115.545 | 209.104 | 29.967 | 1.00 | 50.00 | AAAA | N |
| ATOM | 3407 | OD1 | ASN | A | 360 | 114.660 | 209.930 | 31.885 | 1.00 | 50.00 | AAAA | O |
| ATOM | 3408 | H | ASN | A | 360 | 115.833 | 213.973 | 29.732 | 0.00 | 0.00 | AAAA | H |
| ATOM | 3409 | 1HD2 | ASN | A | 360 | 115.473 | 208.159 | 30.284 | 0.00 | 0.00 | AAAA | H |
| ATOM | 3410 | 2HD2 | ASN | A | 360 | 115.910 | 209.296 | 29.054 | 0.00 | 0.00 | AAAA | H |
| ATOM | 3411 | N | GLU | A | 361 | 118.129 | 212.562 | 31.579 | 1.00 | 50.00 | AAAA | N |
| ATOM | 3412 | CA | GLU | A | 361 | 119.355 | 212.260 | 32.311 | 1.00 | 50.00 | AAAA | C |
| ATOM | 3413 | C | GLU | A | 361 | 119.409 | 212.945 | 33.669 | 1.00 | 50.00 | AAAA | C |
| ATOM | 3414 | O | GLU | A | 361 | 119.778 | 212.362 | 34.681 | 1.00 | 50.00 | AAAA | O |
| ATOM | 3415 | CB | GLU | A | 361 | 120.564 | 212.613 | 31.450 | 1.00 | 50.00 | AAAA | C |
| ATOM | 3416 | CG | GLU | A | 361 | 121.893 | 212.173 | 32.063 | 1.00 | 50.00 | AAAA | C |
| ATOM | 3417 | CD | GLU | A | 361 | 123.032 | 212.551 | 31.140 | 1.00 | 50.00 | AAAA | C |
| ATOM | 3418 | OE1 | GLU | A | 361 | 123.214 | 213.738 | 30.878 | 1.00 | 50.00 | AAAA | O |
| ATOM | 3419 | OE2 | GLU | A | 361 | 123.740 | 211.648 | 30.698 | 1.00 | 50.00 | AAAA | O |
| ATOM | 3420 | H | GLU | A | 361 | 118.133 | 213.142 | 30.764 | 0.00 | 0.00 | AAAA | H |
| ATOM | 3421 | N | LYS | A | 362 | 118.969 | 214.216 | 33.632 | 1.00 | 50.00 | AAAA | N |
| ATOM | 3422 | CA | LYS | A | 362 | 118.882 | 215.006 | 34.856 | 1.00 | 50.00 | AAAA | C |
| ATOM | 3423 | C | LYS | A | 362 | 117.912 | 214.438 | 35.886 | 1.00 | 50.00 | AAAA | C |
| ATOM | 3424 | O | LYS | A | 362 | 118.184 | 214.403 | 37.081 | 1.00 | 50.00 | AAAA | O |
| ATOM | 3425 | CB | LYS | A | 362 | 118.531 | 216.452 | 34.488 | 1.00 | 50.00 | AAAA | C |
| ATOM | 3426 | CG | LYS | A | 362 | 118.614 | 217.448 | 35.647 | 1.00 | 50.00 | AAAA | C |
| ATOM | 3427 | CD | LYS | A | 362 | 120.032 | 217.595 | 36.206 | 1.00 | 50.00 | AAAA | C |
| ATOM | 3428 | CE | LYS | A | 362 | 120.101 | 218.551 | 37.401 | 1.00 | 50.00 | AAAA | C |
| ATOM | 3429 | NZ | LYS | A | 362 | 119.339 | 217.994 | 38.529 | 1.00 | 50.00 | AAAA | N |
| ATOM | 3430 | H | LYS | A | 362 | 118.669 | 214.605 | 32.762 | 0.00 | 0.00 | AAAA | H |
| ATOM | 3431 | 1HZ | LYS | A | 362 | 119.391 | 218.644 | 39.341 | 0.00 | 0.00 | AAAA | H |
| ATOM | 3432 | 2HZ | LYS | A | 362 | 119.750 | 217.077 | 38.797 | 0.00 | 0.00 | AAAA | H |
| ATOM | 3433 | 3HZ | LYS | A | 362 | 118.346 | 217.859 | 38.253 | 0.00 | 0.00 | AAAA | H |
| ATOM | 3434 | N | ILE | A | 363 | 116.768 | 213.965 | 35.353 | 1.00 | 50.00 | AAAA | N |
| ATOM | 3435 | CA | ILE | A | 363 | 115.769 | 213.381 | 36.250 | 1.00 | 50.00 | AAAA | C |
| ATOM | 3436 | C | ILE | A | 363 | 116.155 | 212.024 | 36.808 | 1.00 | 50.00 | AAAA | C |
| ATOM | 3437 | O | ILE | A | 363 | 115.743 | 211.659 | 37.897 | 1.00 | 50.00 | AAAA | O |
| ATOM | 3438 | CB | ILE | A | 363 | 114.358 | 213.321 | 35.634 | 1.00 | 50.00 | AAAA | C |
| ATOM | 3439 | CG1 | ILE | A | 363 | 114.237 | 212.293 | 34.503 | 1.00 | 50.00 | AAAA | C |
| ATOM | 3440 | CG2 | ILE | A | 363 | 113.935 | 214.720 | 35.178 | 1.00 | 50.00 | AAAA | C |
| ATOM | 3441 | CD1 | ILE | A | 363 | 112.845 | 212.155 | 33.892 | 1.00 | 50.00 | AAAA | C |
| ATOM | 3442 | H | ILE | A | 363 | 116.637 | 213.978 | 34.362 | 0.00 | 0.00 | AAAA | H |
| ATOM | 3443 | N | ASN | A | 364 | 116.984 | 211.307 | 36.020 | 1.00 | 50.00 | AAAA | N |
| ATOM | 3444 | CA | ASN | A | 364 | 117.467 | 210.001 | 36.467 | 1.00 | 50.00 | AAAA | C |
| ATOM | 3445 | C | ASN | A | 364 | 118.441 | 210.128 | 37.622 | 1.00 | 50.00 | AAAA | C |
| ATOM | 3446 | O | ASN | A | 364 | 118.438 | 209.346 | 38.561 | 1.00 | 50.00 | AAAA | O |
| ATOM | 3447 | CB | ASN | A | 364 | 118.109 | 209.244 | 35.300 | 1.00 | 50.00 | AAAA | C |
| ATOM | 3448 | CG | ASN | A | 364 | 118.387 | 207.806 | 35.691 | 1.00 | 50.00 | AAAA | C |
| ATOM | 3449 | ND2 | ASN | A | 364 | 119.684 | 207.464 | 35.617 | 1.00 | 50.00 | AAAA | N |
| ATOM | 3450 | OD1 | ASN | A | 364 | 117.492 | 207.050 | 36.041 | 1.00 | 50.00 | AAAA | O |
| ATOM | 3451 | H | ASN | A | 364 | 117.282 | 211.679 | 35.141 | 0.00 | 0.00 | AAAA | H |
| ATOM | 3452 | 1HD2 | ASN | A | 364 | 119.967 | 206.547 | 35.896 | 0.00 | 0.00 | AAAA | H |
| ATOM | 3453 | 2HD2 | ASN | A | 364 | 120.378 | 208.108 | 35.294 | 0.00 | 0.00 | AAAA | H |
| ATOM | 3454 | N | GLN | A | 365 | 119.254 | 211.198 | 37.507 | 1.00 | 50.00 | AAAA | N |
| ATOM | 3455 | CA | GLN | A | 365 | 120.158 | 211.520 | 38.607 | 1.00 | 50.00 | AAAA | C |
| ATOM | 3456 | C | GLN | A | 365 | 119.434 | 211.881 | 39.880 | 1.00 | 50.00 | AAAA | C |
| ATOM | 3457 | O | GLN | A | 365 | 119.829 | 211.471 | 40.958 | 1.00 | 50.00 | AAAA | O |
| ATOM | 3458 | CB | GLN | A | 365 | 121.118 | 212.651 | 38.235 | 1.00 | 50.00 | AAAA | C |
| ATOM | 3459 | CG | GLN | A | 365 | 122.061 | 212.321 | 37.075 | 1.00 | 50.00 | AAAA | C |
| ATOM | 3460 | CD | GLN | A | 365 | 122.930 | 211.131 | 37.427 | 1.00 | 50.00 | AAAA | C |
| ATOM | 3461 | NE2 | GLN | A | 365 | 124.022 | 211.455 | 38.140 | 1.00 | 50.00 | AAAA | N |
| ATOM | 3462 | OE1 | GLN | A | 365 | 122.644 | 209.993 | 37.079 | 1.00 | 50.00 | AAAA | O |
| ATOM | 3463 | H | GLN | A | 365 | 119.194 | 211.788 | 36.702 | 0.00 | 0.00 | AAAA | H |
| ATOM | 3464 | 1HE2 | GLN | A | 365 | 124.670 | 210.740 | 38.398 | 0.00 | 0.00 | AAAA | H |
| ATOM | 3465 | 2HE2 | GLN | A | 365 | 124.205 | 212.398 | 38.414 | 0.00 | 0.00 | AAAA | H |
| ATOM | 3466 | N | SER | A | 366 | 118.329 | 212.631 | 39.696 | 1.00 | 50.00 | AAAA | N |
| ATOM | 3467 | CA | SER | A | 366 | 117.456 | 212.902 | 40.835 | 1.00 | 50.00 | AAAA | C |
| ATOM | 3468 | C | SER | A | 366 | 116.845 | 211.650 | 41.458 | 1.00 | 50.00 | AAAA | C |
| ATOM | 3469 | O | SER | A | 366 | 116.853 | 211.478 | 42.666 | 1.00 | 50.00 | AAAA | O |
| ATOM | 3470 | CB | SER | A | 366 | 116.380 | 213.917 | 40.431 | 1.00 | 50.00 | AAAA | C |
| ATOM | 3471 | OG | SER | A | 366 | 115.641 | 214.347 | 41.579 | 1.00 | 50.00 | AAAA | O |
| ATOM | 3472 | H | SER | A | 366 | 118.117 | 212.994 | 38.789 | 0.00 | 0.00 | AAAA | H |
| ATOM | 3473 | HG | SER | A | 366 | 114.951 | 214.921 | 41.271 | 0.00 | 0.00 | AAAA | H |
| ATOM | 3474 | N | LEU | A | 367 | 116.353 | 210.764 | 40.568 | 1.00 | 50.00 | AAAA | N |
| ATOM | 3475 | CA | LEU | A | 367 | 115.779 | 209.488 | 41.014 | 1.00 | 50.00 | AAAA | C |
| ATOM | 3476 | C | LEU | A | 367 | 116.760 | 208.531 | 41.686 | 1.00 | 50.00 | AAAA | C |
| ATOM | 3477 | O | LEU | A | 367 | 116.390 | 207.657 | 42.458 | 1.00 | 50.00 | AAAA | O |
| ATOM | 3478 | CB | LEU | A | 367 | 115.083 | 208.762 | 39.857 | 1.00 | 50.00 | AAAA | C |
| ATOM | 3479 | CG | LEU | A | 367 | 113.835 | 209.455 | 39.304 | 1.00 | 50.00 | AAAA | C |
| ATOM | 3480 | CD1 | LEU | A | 367 | 113.311 | 208.732 | 38.064 | 1.00 | 50.00 | AAAA | C |
| ATOM | 3481 | CD2 | LEU | A | 367 | 112.739 | 209.628 | 40.357 | 1.00 | 50.00 | AAAA | C |
| ATOM | 3482 | H | LEU | A | 367 | 116.419 | 210.980 | 39.597 | 0.00 | 0.00 | AAAA | H |
| ATOM | 3483 | N | ALA | A | 368 | 118.043 | 208.759 | 41.365 | 1.00 | 50.00 | AAAA | N |
| ATOM | 3484 | CA | ALA | A | 368 | 119.101 | 208.015 | 42.040 | 1.00 | 50.00 | AAAA | C |
| ATOM | 3485 | C | ALA | A | 368 | 119.517 | 208.649 | 43.357 | 1.00 | 50.00 | AAAA | C |
| ATOM | 3486 | O | ALA | A | 368 | 119.785 | 208.004 | 44.360 | 1.00 | 50.00 | AAAA | O |
| ATOM | 3487 | CB | ALA | A | 368 | 120.329 | 207.917 | 41.135 | 1.00 | 50.00 | AAAA | C |
| ATOM | 3488 | H | ALA | A | 368 | 118.268 | 209.461 | 40.690 | 0.00 | 0.00 | AAAA | H |
| ATOM | 3489 | N | PHE | A | 369 | 119.535 | 209.985 | 43.307 | 1.00 | 50.00 | AAAA | N |
| ATOM | 3490 | CA | PHE | A | 369 | 119.843 | 210.765 | 44.498 | 1.00 | 50.00 | AAAA | C |
| ATOM | 3491 | C | PHE | A | 369 | 118.819 | 210.575 | 45.604 | 1.00 | 50.00 | AAAA | C |
| ATOM | 3492 | O | PHE | A | 369 | 119.154 | 210.621 | 46.775 | 1.00 | 50.00 | AAAA | O |
| ATOM | 3493 | CB | PHE | A | 369 | 120.017 | 212.239 | 44.107 | 1.00 | 50.00 | AAAA | C |
| ATOM | 3494 | CG | PHE | A | 369 | 120.428 | 213.087 | 45.288 | 1.00 | 50.00 | AAAA | C |
| ATOM | 3495 | CD1 | PHE | A | 369 | 121.784 | 213.133 | 45.680 | 1.00 | 50.00 | AAAA | C |
| ATOM | 3496 | CD2 | PHE | A | 369 | 119.440 | 213.818 | 45.982 | 1.00 | 50.00 | AAAA | C |
| ATOM | 3497 | CE1 | PHE | A | 369 | 122.156 | 213.920 | 46.788 | 1.00 | 50.00 | AAAA | C |
| ATOM | 3498 | CE2 | PHE | A | 369 | 119.809 | 214.604 | 47.090 | 1.00 | 50.00 | AAAA | C |
| ATOM | 3499 | CZ | PHE | A | 369 | 121.164 | 214.646 | 47.482 | 1.00 | 50.00 | AAAA | C |
| ATOM | 3500 | H | PHE | A | 369 | 119.288 | 210.435 | 42.454 | 0.00 | 0.00 | AAAA | H |
| ATOM | 3501 | N | ILE | A | 370 | 117.566 | 210.325 | 45.171 | 1.00 | 50.00 | AAAA | N |
| ATOM | 3502 | CA | ILE | A | 370 | 116.525 | 210.027 | 46.154 | 1.00 | 50.00 | AAAA | C |
| ATOM | 3503 | C | ILE | A | 370 | 116.621 | 208.641 | 46.769 | 1.00 | 50.00 | AAAA | C |
| ATOM | 3504 | O | ILE | A | 370 | 116.229 | 208.445 | 47.910 | 1.00 | 50.00 | AAAA | O |
| ATOM | 3505 | CB | ILE | A | 370 | 115.099 | 210.269 | 45.625 | 1.00 | 50.00 | AAAA | C |
| ATOM | 3506 | CG1 | ILE | A | 370 | 114.704 | 209.284 | 44.523 | 1.00 | 50.00 | AAAA | C |
| ATOM | 3507 | CG2 | ILE | A | 370 | 114.944 | 211.721 | 45.165 | 1.00 | 50.00 | AAAA | C |
| ATOM | 3508 | CD1 | ILE | A | 370 | 113.235 | 209.337 | 44.110 | 1.00 | 50.00 | AAAA | C |
| ATOM | 3509 | H | ILE | A | 370 | 117.377 | 210.297 | 44.190 | 0.00 | 0.00 | AAAA | H |
| ATOM | 3510 | N | ARG | A | 371 | 117.175 | 207.691 | 45.979 | 1.00 | 50.00 | AAAA | N |
| ATOM | 3511 | CA | ARG | A | 371 | 117.328 | 206.336 | 46.517 | 1.00 | 50.00 | AAAA | C |
| ATOM | 3512 | C | ARG | A | 371 | 118.411 | 206.243 | 47.586 | 1.00 | 50.00 | AAAA | C |
| ATOM | 3513 | O | ARG | A | 371 | 118.282 | 205.549 | 48.587 | 1.00 | 50.00 | AAAA | O |
| ATOM | 3514 | CB | ARG | A | 371 | 117.501 | 205.298 | 45.382 | 1.00 | 50.00 | AAAA | C |
| ATOM | 3515 | CG | ARG | A | 371 | 118.917 | 205.195 | 44.801 | 1.00 | 50.00 | AAAA | C |
| ATOM | 3516 | CD | ARG | A | 371 | 119.071 | 204.537 | 43.431 | 1.00 | 50.00 | AAAA | C |
| ATOM | 3517 | NE | ARG | A | 371 | 120.381 | 204.881 | 42.870 | 1.00 | 50.00 | AAAA | N |
| ATOM | 3518 | CZ | ARG | A | 371 | 120.868 | 204.240 | 41.787 | 1.00 | 50.00 | AAAA | C |
| ATOM | 3519 | NH1 | ARG | A | 371 | 120.214 | 203.216 | 41.252 | 1.00 | 50.00 | AAAA | N |
| ATOM | 3520 | NH2 | ARG | A | 371 | 122.014 | 204.633 | 41.235 | 1.00 | 50.00 | AAAA | N |
| ATOM | 3521 | H | ARG | A | 371 | 117.479 | 207.919 | 45.054 | 0.00 | 0.00 | AAAA | H |
| ATOM | 3522 | HE | ARG | A | 371 | 120.895 | 205.651 | 43.251 | 0.00 | 0.00 | AAAA | H |
| ATOM | 3523 | 1HH1 | ARG | A | 371 | 120.585 | 202.747 | 40.449 | 0.00 | 0.00 | AAAA | H |
| ATOM | 3524 | 2HH1 | ARG | A | 371 | 119.351 | 202.912 | 41.652 | 0.00 | 0.00 | AAAA | H |
| ATOM | 3525 | 1HH2 | ARG | A | 371 | 122.368 | 204.155 | 40.429 | 0.00 | 0.00 | AAAA | H |
| ATOM | 3526 | 2HH2 | ARG | A | 371 | 122.528 | 205.400 | 41.618 | 0.00 | 0.00 | AAAA | H |
| ATOM | 3527 | N | LYS | A | 372 | 119.474 | 207.037 | 47.340 | 1.00 | 50.00 | AAAA | N |
| ATOM | 3528 | CA | LYS | A | 372 | 120.536 | 207.155 | 48.336 | 1.00 | 50.00 | AAAA | C |
| ATOM | 3529 | C | LYS | A | 372 | 120.144 | 208.020 | 49.527 | 1.00 | 50.00 | AAAA | C |
| ATOM | 3530 | O | LYS | A | 372 | 120.466 | 207.742 | 50.673 | 1.00 | 50.00 | AAAA | O |
| ATOM | 3531 | CB | LYS | A | 372 | 121.806 | 207.682 | 47.669 | 1.00 | 50.00 | AAAA | C |
| ATOM | 3532 | CG | LYS | A | 372 | 123.018 | 207.618 | 48.597 | 1.00 | 50.00 | AAAA | C |
| ATOM | 3533 | CD | LYS | A | 372 | 124.270 | 208.257 | 48.012 | 1.00 | 50.00 | AAAA | C |
| ATOM | 3534 | CE | LYS | A | 372 | 124.090 | 209.753 | 47.773 | 1.00 | 50.00 | AAAA | C |
| ATOM | 3535 | NZ | LYS | A | 372 | 125.340 | 210.289 | 47.225 | 1.00 | 50.00 | AAAA | N |
| ATOM | 3536 | H | LYS | A | 372 | 119.523 | 207.540 | 46.476 | 0.00 | 0.00 | AAAA | H |
| ATOM | 3537 | 1HZ | LYS | A | 372 | 125.229 | 211.305 | 47.035 | 0.00 | 0.00 | AAAA | H |
| ATOM | 3538 | 2HZ | LYS | A | 372 | 125.560 | 209.792 | 46.338 | 0.00 | 0.00 | AAAA | H |
| ATOM | 3539 | 3HZ | LYS | A | 372 | 126.111 | 210.139 | 47.907 | 0.00 | 0.00 | AAAA | H |
| ATOM | 3540 | N | SER | A | 373 | 119.373 | 209.072 | 49.199 | 1.00 | 50.00 | AAAA | N |
| ATOM | 3541 | CA | SER | A | 373 | 118.726 | 209.879 | 50.240 | 1.00 | 50.00 | AAAA | C |
| ATOM | 3542 | C | SER | A | 373 | 117.812 | 209.064 | 51.153 | 1.00 | 50.00 | AAAA | C |
| ATOM | 3543 | O | SER | A | 373 | 117.693 | 209.303 | 52.349 | 1.00 | 50.00 | AAAA | O |
| ATOM | 3544 | CB | SER | A | 373 | 117.964 | 211.026 | 49.566 | 1.00 | 50.00 | AAAA | C |
| ATOM | 3545 | OG | SER | A | 373 | 117.371 | 211.913 | 50.516 | 1.00 | 50.00 | AAAA | O |
| ATOM | 3546 | H | SER | A | 373 | 119.247 | 209.286 | 48.230 | 0.00 | 0.00 | AAAA | H |
| ATOM | 3547 | HG | SER | A | 373 | 116.894 | 212.557 | 50.007 | 0.00 | 0.00 | AAAA | H |
| ATOM | 3548 | N | ASP | A | 374 | 117.204 | 208.046 | 50.511 | 1.00 | 50.00 | AAAA | N |
| ATOM | 3549 | CA | ASP | A | 374 | 116.323 | 207.122 | 51.226 | 1.00 | 50.00 | AAAA | C |
| ATOM | 3550 | C | ASP | A | 374 | 117.051 | 206.197 | 52.199 | 1.00 | 50.00 | AAAA | C |
| ATOM | 3551 | O | ASP | A | 374 | 116.451 | 205.559 | 53.054 | 1.00 | 50.00 | AAAA | O |
| ATOM | 3552 | CB | ASP | A | 374 | 115.507 | 206.320 | 50.206 | 1.00 | 50.00 | AAAA | C |
| ATOM | 3553 | CG | ASP | A | 374 | 114.140 | 205.950 | 50.745 | 1.00 | 50.00 | AAAA | C |
| ATOM | 3554 | OD1 | ASP | A | 374 | 114.045 | 205.026 | 51.549 | 1.00 | 50.00 | AAAA | O |
| ATOM | 3555 | OD2 | ASP | A | 374 | 113.165 | 206.579 | 50.339 | 1.00 | 50.00 | AAAA | O |
| ATOM | 3556 | H | ASP | A | 374 | 117.392 | 207.899 | 49.540 | 0.00 | 0.00 | AAAA | H |
| ATOM | 3557 | N | GLU | A | 375 | 118.389 | 206.165 | 52.036 | 1.00 | 50.00 | AAAA | N |
| ATOM | 3558 | CA | GLU | A | 375 | 119.196 | 205.347 | 52.937 | 1.00 | 50.00 | AAAA | C |
| ATOM | 3559 | C | GLU | A | 375 | 119.608 | 206.096 | 54.199 | 1.00 | 50.00 | AAAA | C |
| ATOM | 3560 | O | GLU | A | 375 | 119.652 | 205.542 | 55.290 | 1.00 | 50.00 | AAAA | O |
| ATOM | 3561 | CB | GLU | A | 375 | 120.382 | 204.724 | 52.169 | 1.00 | 50.00 | AAAA | C |
| ATOM | 3562 | CG | GLU | A | 375 | 121.733 | 205.446 | 52.317 | 1.00 | 50.00 | AAAA | C |
| ATOM | 3563 | CD | GLU | A | 375 | 122.700 | 205.205 | 51.171 | 1.00 | 50.00 | AAAA | C |
| ATOM | 3564 | OE1 | GLU | A | 375 | 122.305 | 204.655 | 50.144 | 1.00 | 50.00 | AAAA | O |
| ATOM | 3565 | OE2 | GLU | A | 375 | 123.861 | 205.588 | 51.318 | 1.00 | 50.00 | AAAA | O |
| ATOM | 3566 | H | GLU | A | 375 | 118.824 | 206.775 | 51.376 | 0.00 | 0.00 | AAAA | H |
| ATOM | 3567 | N | LEU | A | 376 | 119.865 | 207.409 | 54.001 | 1.00 | 50.00 | AAAA | N |
| ATOM | 3568 | CA | LEU | A | 376 | 120.156 | 208.283 | 55.140 | 1.00 | 50.00 | AAAA | C |
| ATOM | 3569 | C | LEU | A | 376 | 118.943 | 208.494 | 56.042 | 1.00 | 50.00 | AAAA | C |
| ATOM | 3570 | O | LEU | A | 376 | 119.056 | 208.825 | 57.215 | 1.00 | 50.00 | AAAA | O |
| ATOM | 3571 | CB | LEU | A | 376 | 120.727 | 209.630 | 54.663 | 1.00 | 50.00 | AAAA | C |
| ATOM | 3572 | CG | LEU | A | 376 | 122.231 | 209.675 | 54.332 | 1.00 | 50.00 | AAAA | C |
| ATOM | 3573 | CD1 | LEU | A | 376 | 122.638 | 208.864 | 53.102 | 1.00 | 50.00 | AAAA | C |
| ATOM | 3574 | CD2 | LEU | A | 376 | 122.716 | 211.118 | 54.187 | 1.00 | 50.00 | AAAA | C |
| ATOM | 3575 | H | LEU | A | 376 | 119.828 | 207.789 | 53.077 | 0.00 | 0.00 | AAAA | H |
| ATOM | 3576 | N | LEU | A | 377 | 117.769 | 208.236 | 55.426 | 1.00 | 50.00 | AAAA | N |
| ATOM | 3577 | CA | LEU | A | 377 | 116.511 | 208.221 | 56.173 | 1.00 | 50.00 | AAAA | C |
| ATOM | 3578 | C | LEU | A | 377 | 116.271 | 206.962 | 56.991 | 1.00 | 50.00 | AAAA | C |
| ATOM | 3579 | O | LEU | A | 377 | 115.710 | 206.989 | 58.079 | 1.00 | 50.00 | AAAA | O |
| ATOM | 3580 | CB | LEU | A | 377 | 115.333 | 208.436 | 55.227 | 1.00 | 50.00 | AAAA | C |
| ATOM | 3581 | CG | LEU | A | 377 | 115.266 | 209.847 | 54.641 | 1.00 | 50.00 | AAAA | C |
| ATOM | 3582 | CD1 | LEU | A | 377 | 114.163 | 209.957 | 53.588 | 1.00 | 50.00 | AAAA | C |
| ATOM | 3583 | CD2 | LEU | A | 377 | 115.135 | 210.913 | 55.731 | 1.00 | 50.00 | AAAA | C |
| ATOM | 3584 | H | LEU | A | 377 | 117.773 | 208.003 | 54.455 | 0.00 | 0.00 | AAAA | H |
| ATOM | 3585 | N | HIS | A | 378 | 116.744 | 205.838 | 56.426 | 1.00 | 50.00 | AAAA | N |
| ATOM | 3586 | CA | HIS | A | 378 | 116.650 | 204.602 | 57.204 | 1.00 | 50.00 | AAAA | C |
| ATOM | 3587 | C | HIS | A | 378 | 117.564 | 204.613 | 58.429 | 1.00 | 50.00 | AAAA | C |
| ATOM | 3588 | O | HIS | A | 378 | 117.263 | 204.061 | 59.480 | 1.00 | 50.00 | AAAA | O |
| ATOM | 3589 | CB | HIS | A | 378 | 116.913 | 203.417 | 56.266 | 1.00 | 50.00 | AAAA | C |
| ATOM | 3590 | CG | HIS | A | 378 | 116.587 | 202.075 | 56.890 | 1.00 | 50.00 | AAAA | C |
| ATOM | 3591 | CD2 | HIS | A | 378 | 115.758 | 201.787 | 57.982 | 1.00 | 50.00 | AAAA | C |
| ATOM | 3592 | ND1 | HIS | A | 378 | 117.093 | 200.920 | 56.418 | 1.00 | 50.00 | AAAA | N |
| ATOM | 3593 | CE1 | HIS | A | 378 | 116.596 | 199.910 | 57.201 | 1.00 | 50.00 | AAAA | C |
| ATOM | 3594 | NE2 | HIS | A | 378 | 115.777 | 200.443 | 58.161 | 1.00 | 50.00 | AAAA | N |
| ATOM | 3595 | H | HIS | A | 378 | 117.192 | 205.856 | 55.533 | 0.00 | 0.00 | AAAA | H |
| ATOM | 3596 | HD1 | HIS | A | 378 | 117.695 | 200.819 | 55.652 | 0.00 | 0.00 | AAAA | H |
| ATOM | 3597 | N | ASN | A | 379 | 118.689 | 205.329 | 58.240 | 1.00 | 50.00 | AAAA | N |
| ATOM | 3598 | CA | ASN | A | 379 | 119.614 | 205.525 | 59.352 | 1.00 | 50.00 | AAAA | C |
| ATOM | 3599 | C | ASN | A | 379 | 119.042 | 206.379 | 60.478 | 1.00 | 50.00 | AAAA | C |
| ATOM | 3600 | O | ASN | A | 379 | 119.230 | 206.077 | 61.649 | 1.00 | 50.00 | AAAA | O |
| ATOM | 3601 | CB | ASN | A | 379 | 120.940 | 206.081 | 58.813 | 1.00 | 50.00 | AAAA | C |
| ATOM | 3602 | CG | ASN | A | 379 | 122.029 | 206.066 | 59.874 | 1.00 | 50.00 | AAAA | C |
| ATOM | 3603 | ND2 | ASN | A | 379 | 123.076 | 205.288 | 59.563 | 1.00 | 50.00 | AAAA | N |
| ATOM | 3604 | OD1 | ASN | A | 379 | 121.951 | 206.727 | 60.900 | 1.00 | 50.00 | AAAA | O |
| ATOM | 3605 | H | ASN | A | 379 | 118.850 | 205.766 | 57.355 | 0.00 | 0.00 | AAAA | H |
| ATOM | 3606 | 1HD2 | ASN | A | 379 | 123.843 | 205.229 | 60.201 | 0.00 | 0.00 | AAAA | H |
| ATOM | 3607 | 2HD2 | ASN | A | 379 | 123.112 | 204.775 | 58.706 | 0.00 | 0.00 | AAAA | H |
| ATOM | 3608 | N | VAL | A | 380 | 118.315 | 207.446 | 60.078 | 1.00 | 50.00 | AAAA | N |
| ATOM | 3609 | CA | VAL | A | 380 | 117.747 | 208.296 | 61.130 | 1.00 | 50.00 | AAAA | C |
| ATOM | 3610 | C | VAL | A | 380 | 116.701 | 207.606 | 61.998 | 1.00 | 50.00 | AAAA | C |
| ATOM | 3611 | O | VAL | A | 380 | 116.682 | 207.763 | 63.208 | 1.00 | 50.00 | AAAA | O |
| ATOM | 3612 | CB | VAL | A | 380 | 117.264 | 209.664 | 60.591 | 1.00 | 50.00 | AAAA | C |
| ATOM | 3613 | CG1 | VAL | A | 380 | 116.067 | 209.587 | 59.644 | 1.00 | 50.00 | AAAA | C |
| ATOM | 3614 | CG2 | VAL | A | 380 | 116.983 | 210.643 | 61.732 | 1.00 | 50.00 | AAAA | C |
| ATOM | 3615 | H | VAL | A | 380 | 118.184 | 207.641 | 59.106 | 0.00 | 0.00 | AAAA | H |
| ATOM | 3616 | N | ASN | A | 381 | 115.877 | 206.776 | 61.324 | 1.00 | 50.00 | AAAA | N |
| ATOM | 3617 | CA | ASN | A | 381 | 114.896 | 205.952 | 62.035 | 1.00 | 50.00 | AAAA | C |
| ATOM | 3618 | C | ASN | A | 381 | 115.527 | 205.024 | 63.070 | 1.00 | 50.00 | AAAA | C |
| ATOM | 3619 | O | ASN | A | 381 | 114.972 | 204.755 | 64.126 | 1.00 | 50.00 | AAAA | O |
| ATOM | 3620 | CB | ASN | A | 381 | 114.076 | 205.166 | 61.003 | 1.00 | 50.00 | AAAA | C |
| ATOM | 3621 | CG | ASN | A | 381 | 112.882 | 204.481 | 61.642 | 1.00 | 50.00 | AAAA | C |
| ATOM | 3622 | ND2 | ASN | A | 381 | 112.892 | 203.145 | 61.499 | 1.00 | 50.00 | AAAA | N |
| ATOM | 3623 | OD1 | ASN | A | 381 | 112.004 | 205.108 | 62.217 | 1.00 | 50.00 | AAAA | O |
| ATOM | 3624 | H | ASN | A | 381 | 115.953 | 206.717 | 60.327 | 0.00 | 0.00 | AAAA | H |
| ATOM | 3625 | 1HD2 | ASN | A | 381 | 112.161 | 202.599 | 61.905 | 0.00 | 0.00 | AAAA | H |
| ATOM | 3626 | 2HD2 | ASN | A | 381 | 113.615 | 202.682 | 60.985 | 0.00 | 0.00 | AAAA | H |
| ATOM | 3627 | N | ALA | A | 382 | 116.742 | 204.569 | 62.705 | 1.00 | 50.00 | AAAA | N |
| ATOM | 3628 | CA | ALA | A | 382 | 117.494 | 203.724 | 63.626 | 1.00 | 50.00 | AAAA | C |
| ATOM | 3629 | C | ALA | A | 382 | 118.046 | 204.467 | 64.833 | 1.00 | 50.00 | AAAA | C |
| ATOM | 3630 | O | ALA | A | 382 | 117.760 | 204.109 | 65.964 | 1.00 | 50.00 | AAAA | O |
| ATOM | 3631 | CB | ALA | A | 382 | 118.637 | 203.019 | 62.894 | 1.00 | 50.00 | AAAA | C |
| ATOM | 3632 | H | ALA | A | 382 | 117.141 | 204.869 | 61.837 | 0.00 | 0.00 | AAAA | H |
| ATOM | 3633 | N | LYS | A | 383 | 118.813 | 205.539 | 64.540 | 1.00 | 50.00 | AAAA | N |
| ATOM | 3634 | CA | LYS | A | 383 | 119.339 | 206.418 | 65.593 | 1.00 | 50.00 | AAAA | C |
| ATOM | 3635 | C | LYS | A | 383 | 118.279 | 207.104 | 66.464 | 1.00 | 50.00 | AAAA | C |
| ATOM | 3636 | O | LYS | A | 383 | 118.528 | 207.593 | 67.559 | 1.00 | 50.00 | AAAA | O |
| ATOM | 3637 | CB | LYS | A | 383 | 120.296 | 207.431 | 64.948 | 1.00 | 50.00 | AAAA | C |
| ATOM | 3638 | CG | LYS | A | 383 | 121.129 | 208.287 | 65.910 | 1.00 | 50.00 | AAAA | C |
| ATOM | 3639 | CD | LYS | A | 383 | 122.092 | 207.480 | 66.783 | 1.00 | 50.00 | AAAA | C |
| ATOM | 3640 | CE | LYS | A | 383 | 122.844 | 208.374 | 67.770 | 1.00 | 50.00 | AAAA | C |
| ATOM | 3641 | NZ | LYS | A | 383 | 123.797 | 207.570 | 68.548 | 1.00 | 50.00 | AAAA | N |
| ATOM | 3642 | H | LYS | A | 383 | 119.026 | 205.739 | 63.585 | 0.00 | 0.00 | AAAA | H |
| ATOM | 3643 | 1HZ | LYS | A | 383 | 124.288 | 208.177 | 69.237 | 0.00 | 0.00 | AAAA | H |
| ATOM | 3644 | 2HZ | LYS | A | 383 | 124.497 | 207.151 | 67.902 | 0.00 | 0.00 | AAAA | H |
| ATOM | 3645 | 3HZ | LYS | A | 383 | 123.296 | 206.810 | 69.052 | 0.00 | 0.00 | AAAA | H |
| ATOM | 3646 | N | LYS | A | 384 | 117.054 | 207.094 | 65.914 | 1.00 | 50.00 | AAAA | N |
| ATOM | 3647 | CA | LYS | A | 384 | 115.935 | 207.595 | 66.696 | 1.00 | 50.00 | AAAA | C |
| ATOM | 3648 | C | LYS | A | 384 | 115.392 | 206.553 | 67.651 | 1.00 | 50.00 | AAAA | C |
| ATOM | 3649 | O | LYS | A | 384 | 115.134 | 206.838 | 68.810 | 1.00 | 50.00 | AAAA | O |
| ATOM | 3650 | CB | LYS | A | 384 | 114.851 | 208.115 | 65.760 | 1.00 | 50.00 | AAAA | C |
| ATOM | 3651 | CG | LYS | A | 384 | 113.722 | 208.869 | 66.458 | 1.00 | 50.00 | AAAA | C |
| ATOM | 3652 | CD | LYS | A | 384 | 112.663 | 209.325 | 65.459 | 1.00 | 50.00 | AAAA | C |
| ATOM | 3653 | CE | LYS | A | 384 | 112.043 | 208.152 | 64.698 | 1.00 | 50.00 | AAAA | C |
| ATOM | 3654 | NZ | LYS | A | 384 | 111.054 | 208.670 | 63.744 | 1.00 | 50.00 | AAAA | N |
| ATOM | 3655 | H | LYS | A | 384 | 116.896 | 206.664 | 65.026 | 0.00 | 0.00 | AAAA | H |
| ATOM | 3656 | 1HZ | LYS | A | 384 | 110.596 | 207.881 | 63.246 | 0.00 | 0.00 | AAAA | H |
| ATOM | 3657 | 2HZ | LYS | A | 384 | 111.527 | 209.290 | 63.055 | 0.00 | 0.00 | AAAA | H |
| ATOM | 3658 | 3HZ | LYS | A | 384 | 110.335 | 209.216 | 64.263 | 0.00 | 0.00 | AAAA | H |
| ATOM | 3659 | N | SER | A | 385 | 115.277 | 205.316 | 67.122 | 1.00 | 50.00 | AAAA | N |
| ATOM | 3660 | CA | SER | A | 385 | 114.859 | 204.220 | 67.999 | 1.00 | 50.00 | AAAA | C |
| ATOM | 3661 | C | SER | A | 385 | 115.848 | 203.929 | 69.118 | 1.00 | 50.00 | AAAA | C |
| ATOM | 3662 | O | SER | A | 385 | 115.483 | 203.553 | 70.224 | 1.00 | 50.00 | AAAA | O |
| ATOM | 3663 | CB | SER | A | 385 | 114.585 | 202.953 | 67.182 | 1.00 | 50.00 | AAAA | C |
| ATOM | 3664 | OG | SER | A | 385 | 113.514 | 203.191 | 66.264 | 1.00 | 50.00 | AAAA | O |
| ATOM | 3665 | H | SER | A | 385 | 115.533 | 205.134 | 66.173 | 0.00 | 0.00 | AAAA | H |
| ATOM | 3666 | HG | SER | A | 385 | 113.507 | 202.469 | 65.645 | 0.00 | 0.00 | AAAA | H |
| ATOM | 3667 | N | THR | A | 386 | 117.131 | 204.166 | 68.776 | 1.00 | 50.00 | AAAA | N |
| ATOM | 3668 | CA | THR | A | 386 | 118.185 | 204.034 | 69.778 | 1.00 | 50.00 | AAAA | C |
| ATOM | 3669 | C | THR | A | 386 | 118.104 | 205.091 | 70.861 | 1.00 | 50.00 | AAAA | C |
| ATOM | 3670 | O | THR | A | 386 | 118.251 | 204.809 | 72.040 | 1.00 | 50.00 | AAAA | O |
| ATOM | 3671 | CB | THR | A | 386 | 119.572 | 204.066 | 69.135 | 1.00 | 50.00 | AAAA | C |
| ATOM | 3672 | CG2 | THR | A | 386 | 119.785 | 202.920 | 68.146 | 1.00 | 50.00 | AAAA | C |
| ATOM | 3673 | OG1 | THR | A | 386 | 119.785 | 205.328 | 68.501 | 1.00 | 50.00 | AAAA | O |
| ATOM | 3674 | H | THR | A | 386 | 117.349 | 204.515 | 67.867 | 0.00 | 0.00 | AAAA | H |
| ATOM | 3675 | HG1 | THR | A | 386 | 120.676 | 205.340 | 68.181 | 0.00 | 0.00 | AAAA | H |
| ATOM | 3676 | N | THR | A | 387 | 117.828 | 206.329 | 70.412 | 1.00 | 50.00 | AAAA | N |
| ATOM | 3677 | CA | THR | A | 387 | 117.644 | 207.379 | 71.409 | 1.00 | 50.00 | AAAA | C |
| ATOM | 3678 | C | THR | A | 387 | 116.456 | 207.115 | 72.314 | 1.00 | 50.00 | AAAA | C |
| ATOM | 3679 | O | THR | A | 387 | 116.563 | 207.184 | 73.527 | 1.00 | 50.00 | AAAA | O |
| ATOM | 3680 | CB | THR | A | 387 | 117.571 | 208.753 | 70.731 | 1.00 | 50.00 | AAAA | C |
| ATOM | 3681 | CG2 | THR | A | 387 | 117.267 | 209.906 | 71.694 | 1.00 | 50.00 | AAAA | C |
| ATOM | 3682 | OG1 | THR | A | 387 | 118.811 | 209.005 | 70.070 | 1.00 | 50.00 | AAAA | O |
| ATOM | 3683 | H | THR | A | 387 | 117.768 | 206.521 | 69.431 | 0.00 | 0.00 | AAAA | H |
| ATOM | 3684 | HG1 | THR | A | 387 | 118.681 | 209.769 | 69.522 | 0.00 | 0.00 | AAAA | H |
| ATOM | 3685 | N | ASN | A | 388 | 115.336 | 206.745 | 71.671 | 1.00 | 50.00 | AAAA | N |
| ATOM | 3686 | CA | ASN | A | 388 | 114.133 | 206.444 | 72.443 | 1.00 | 50.00 | AAAA | C |
| ATOM | 3687 | C | ASN | A | 388 | 114.307 | 205.334 | 73.475 | 1.00 | 50.00 | AAAA | C |
| ATOM | 3688 | O | ASN | A | 388 | 113.717 | 205.391 | 74.544 | 1.00 | 50.00 | AAAA | O |
| ATOM | 3689 | CB | ASN | A | 388 | 112.927 | 206.166 | 71.531 | 1.00 | 50.00 | AAAA | C |
| ATOM | 3690 | CG | ASN | A | 388 | 112.534 | 207.386 | 70.706 | 1.00 | 50.00 | AAAA | C |
| ATOM | 3691 | ND2 | ASN | A | 388 | 112.096 | 208.433 | 71.431 | 1.00 | 50.00 | AAAA | N |
| ATOM | 3692 | OD1 | ASN | A | 388 | 112.591 | 207.386 | 69.484 | 1.00 | 50.00 | AAAA | O |
| ATOM | 3693 | H | ASN | A | 388 | 115.342 | 206.656 | 70.678 | 0.00 | 0.00 | AAAA | H |
| ATOM | 3694 | 1HD2 | ASN | A | 388 | 111.805 | 209.255 | 70.941 | 0.00 | 0.00 | AAAA | H |
| ATOM | 3695 | 2HD2 | ASN | A | 388 | 112.042 | 208.417 | 72.430 | 0.00 | 0.00 | AAAA | H |
| ATOM | 3696 | N | ILE | A | 389 | 115.172 | 204.350 | 73.138 | 1.00 | 50.00 | AAAA | N |
| ATOM | 3697 | CA | ILE | A | 389 | 115.435 | 203.293 | 74.123 | 1.00 | 50.00 | AAAA | C |
| ATOM | 3698 | C | ILE | A | 389 | 116.387 | 203.678 | 75.247 | 1.00 | 50.00 | AAAA | C |
| ATOM | 3699 | O | ILE | A | 389 | 116.170 | 203.362 | 76.408 | 1.00 | 50.00 | AAAA | O |
| ATOM | 3700 | CB | ILE | A | 389 | 115.874 | 201.958 | 73.483 | 1.00 | 50.00 | AAAA | C |
| ATOM | 3701 | CG1 | ILE | A | 389 | 117.230 | 202.029 | 72.771 | 1.00 | 50.00 | AAAA | C |
| ATOM | 3702 | CG2 | ILE | A | 389 | 114.773 | 201.458 | 72.545 | 1.00 | 50.00 | AAAA | C |
| ATOM | 3703 | CD1 | ILE | A | 389 | 117.784 | 200.706 | 72.246 | 1.00 | 50.00 | AAAA | C |
| ATOM | 3704 | H | ILE | A | 389 | 115.659 | 204.387 | 72.264 | 0.00 | 0.00 | AAAA | H |
| ATOM | 3705 | N | MET | A | 390 | 117.452 | 204.405 | 74.861 | 1.00 | 50.00 | AAAA | N |
| ATOM | 3706 | CA | MET | A | 390 | 118.387 | 204.876 | 75.880 | 1.00 | 50.00 | AAAA | C |
| ATOM | 3707 | C | MET | A | 390 | 117.756 | 205.865 | 76.843 | 1.00 | 50.00 | AAAA | C |
| ATOM | 3708 | O | MET | A | 390 | 118.076 | 205.910 | 78.018 | 1.00 | 50.00 | AAAA | O |
| ATOM | 3709 | CB | MET | A | 390 | 119.651 | 205.447 | 75.232 | 1.00 | 50.00 | AAAA | C |
| ATOM | 3710 | CG | MET | A | 390 | 120.437 | 204.380 | 74.462 | 1.00 | 50.00 | AAAA | C |
| ATOM | 3711 | SD | MET | A | 390 | 121.949 | 204.996 | 73.694 | 1.00 | 50.00 | AAAA | S |
| ATOM | 3712 | CE | MET | A | 390 | 121.219 | 206.075 | 72.451 | 1.00 | 50.00 | AAAA | C |
| ATOM | 3713 | H | MET | A | 390 | 117.603 | 204.608 | 73.895 | 0.00 | 0.00 | AAAA | H |
| ATOM | 3714 | N | ILE | A | 391 | 116.788 | 206.614 | 76.289 | 1.00 | 50.00 | AAAA | N |
| ATOM | 3715 | CA | ILE | A | 391 | 115.963 | 207.485 | 77.122 | 1.00 | 50.00 | AAAA | C |
| ATOM | 3716 | C | ILE | A | 391 | 115.064 | 206.695 | 78.069 | 1.00 | 50.00 | AAAA | C |
| ATOM | 3717 | O | ILE | A | 391 | 114.963 | 207.021 | 79.242 | 1.00 | 50.00 | AAAA | O |
| ATOM | 3718 | CB | ILE | A | 391 | 115.170 | 208.478 | 76.245 | 1.00 | 50.00 | AAAA | C |
| ATOM | 3719 | CG1 | ILE | A | 391 | 116.114 | 209.426 | 75.492 | 1.00 | 50.00 | AAAA | C |
| ATOM | 3720 | CG2 | ILE | A | 391 | 114.166 | 209.311 | 77.051 | 1.00 | 50.00 | AAAA | C |
| ATOM | 3721 | CD1 | ILE | A | 391 | 117.012 | 210.287 | 76.383 | 1.00 | 50.00 | AAAA | C |
| ATOM | 3722 | H | ILE | A | 391 | 116.580 | 206.481 | 75.324 | 0.00 | 0.00 | AAAA | H |
| ATOM | 3723 | N | THR | A | 392 | 114.453 | 205.617 | 77.527 | 1.00 | 50.00 | AAAA | N |
| ATOM | 3724 | CA | THR | A | 392 | 113.612 | 204.779 | 78.393 | 1.00 | 50.00 | AAAA | C |
| ATOM | 3725 | C | THR | A | 392 | 114.343 | 204.041 | 79.502 | 1.00 | 50.00 | AAAA | C |
| ATOM | 3726 | O | THR | A | 392 | 113.764 | 203.643 | 80.504 | 1.00 | 50.00 | AAAA | O |
| ATOM | 3727 | CB | THR | A | 392 | 112.766 | 203.765 | 77.610 | 1.00 | 50.00 | AAAA | C |
| ATOM | 3728 | CG2 | THR | A | 392 | 111.734 | 204.425 | 76.699 | 1.00 | 50.00 | AAAA | C |
| ATOM | 3729 | OG1 | THR | A | 392 | 113.599 | 202.860 | 76.882 | 1.00 | 50.00 | AAAA | O |
| ATOM | 3730 | H | THR | A | 392 | 114.608 | 205.360 | 76.573 | 0.00 | 0.00 | AAAA | H |
| ATOM | 3731 | HG1 | THR | A | 392 | 113.032 | 202.194 | 76.517 | 0.00 | 0.00 | AAAA | H |
| ATOM | 3732 | N | THR | A | 393 | 115.656 | 203.882 | 79.285 | 1.00 | 50.00 | AAAA | N |
| ATOM | 3733 | CA | THR | A | 393 | 116.429 | 203.252 | 80.345 | 1.00 | 50.00 | AAAA | C |
| ATOM | 3734 | C | THR | A | 393 | 117.019 | 204.246 | 81.324 | 1.00 | 50.00 | AAAA | C |
| ATOM | 3735 | O | THR | A | 393 | 117.122 | 203.990 | 82.513 | 1.00 | 50.00 | AAAA | O |
| ATOM | 3736 | CB | THR | A | 393 | 117.512 | 202.353 | 79.762 | 1.00 | 50.00 | AAAA | C |
| ATOM | 3737 | CG2 | THR | A | 393 | 116.908 | 201.212 | 78.940 | 1.00 | 50.00 | AAAA | C |
| ATOM | 3738 | OG1 | THR | A | 393 | 118.423 | 203.122 | 78.972 | 1.00 | 50.00 | AAAA | O |
| ATOM | 3739 | H | THR | A | 393 | 116.091 | 204.200 | 78.443 | 0.00 | 0.00 | AAAA | H |
| ATOM | 3740 | HG1 | THR | A | 393 | 119.070 | 202.515 | 78.642 | 0.00 | 0.00 | AAAA | H |
| ATOM | 3741 | N | ILE | A | 394 | 117.362 | 205.424 | 80.776 | 1.00 | 50.00 | AAAA | N |
| ATOM | 3742 | CA | ILE | A | 394 | 117.770 | 206.522 | 81.651 | 1.00 | 50.00 | AAAA | C |
| ATOM | 3743 | C | ILE | A | 394 | 116.653 | 206.975 | 82.589 | 1.00 | 50.00 | AAAA | C |
| ATOM | 3744 | O | ILE | A | 394 | 116.887 | 207.364 | 83.724 | 1.00 | 50.00 | AAAA | O |
| ATOM | 3745 | CB | ILE | A | 394 | 118.392 | 207.673 | 80.830 | 1.00 | 50.00 | AAAA | C |
| ATOM | 3746 | CG1 | ILE | A | 394 | 119.714 | 207.194 | 80.220 | 1.00 | 50.00 | AAAA | C |
| ATOM | 3747 | CG2 | ILE | A | 394 | 118.681 | 208.924 | 81.668 | 1.00 | 50.00 | AAAA | C |
| ATOM | 3748 | CD1 | ILE | A | 394 | 120.293 | 208.183 | 79.204 | 1.00 | 50.00 | AAAA | C |
| ATOM | 3749 | H | ILE | A | 394 | 117.311 | 205.534 | 79.785 | 0.00 | 0.00 | AAAA | H |
| ATOM | 3750 | N | ILE | A | 395 | 115.408 | 206.842 | 82.095 | 1.00 | 50.00 | AAAA | N |
| ATOM | 3751 | CA | ILE | A | 395 | 114.319 | 207.174 | 83.013 | 1.00 | 50.00 | AAAA | C |
| ATOM | 3752 | C | ILE | A | 395 | 114.117 | 206.169 | 84.136 | 1.00 | 50.00 | AAAA | C |
| ATOM | 3753 | O | ILE | A | 395 | 113.807 | 206.545 | 85.258 | 1.00 | 50.00 | AAAA | O |
| ATOM | 3754 | CB | ILE | A | 395 | 112.997 | 207.477 | 82.287 | 1.00 | 50.00 | AAAA | C |
| ATOM | 3755 | CG1 | ILE | A | 395 | 112.424 | 206.250 | 81.574 | 1.00 | 50.00 | AAAA | C |
| ATOM | 3756 | CG2 | ILE | A | 395 | 113.208 | 208.651 | 81.327 | 1.00 | 50.00 | AAAA | C |
| ATOM | 3757 | CD1 | ILE | A | 395 | 111.095 | 206.470 | 80.854 | 1.00 | 50.00 | AAAA | C |
| ATOM | 3758 | H | ILE | A | 395 | 115.247 | 206.465 | 81.184 | 0.00 | 0.00 | AAAA | H |
| ATOM | 3759 | N | ILE | A | 396 | 114.341 | 204.878 | 83.802 | 1.00 | 50.00 | AAAA | N |
| ATOM | 3760 | CA | ILE | A | 396 | 114.189 | 203.867 | 84.850 | 1.00 | 50.00 | AAAA | C |
| ATOM | 3761 | C | ILE | A | 396 | 115.320 | 203.868 | 85.863 | 1.00 | 50.00 | AAAA | C |
| ATOM | 3762 | O | ILE | A | 396 | 115.120 | 203.612 | 87.041 | 1.00 | 50.00 | AAAA | O |
| ATOM | 3763 | CB | ILE | A | 396 | 113.940 | 202.451 | 84.291 | 1.00 | 50.00 | AAAA | C |
| ATOM | 3764 | CG1 | ILE | A | 396 | 115.155 | 201.856 | 83.567 | 1.00 | 50.00 | AAAA | C |
| ATOM | 3765 | CG2 | ILE | A | 396 | 112.705 | 202.479 | 83.387 | 1.00 | 50.00 | AAAA | C |
| ATOM | 3766 | CD1 | ILE | A | 396 | 114.976 | 200.428 | 83.052 | 1.00 | 50.00 | AAAA | C |
| ATOM | 3767 | H | ILE | A | 396 | 114.640 | 204.632 | 82.881 | 0.00 | 0.00 | AAAA | H |
| ATOM | 3768 | N | VAL | A | 397 | 116.516 | 204.212 | 85.343 | 1.00 | 50.00 | AAAA | N |
| ATOM | 3769 | CA | VAL | A | 397 | 117.681 | 204.306 | 86.216 | 1.00 | 50.00 | AAAA | C |
| ATOM | 3770 | C | VAL | A | 397 | 117.659 | 205.524 | 87.129 | 1.00 | 50.00 | AAAA | C |
| ATOM | 3771 | O | VAL | A | 397 | 118.218 | 205.525 | 88.216 | 1.00 | 50.00 | AAAA | O |
| ATOM | 3772 | CB | VAL | A | 397 | 118.995 | 204.179 | 85.412 | 1.00 | 50.00 | AAAA | C |
| ATOM | 3773 | CG1 | VAL | A | 397 | 119.327 | 205.415 | 84.580 | 1.00 | 50.00 | AAAA | C |
| ATOM | 3774 | CG2 | VAL | A | 397 | 120.173 | 203.792 | 86.309 | 1.00 | 50.00 | AAAA | C |
| ATOM | 3775 | H | VAL | A | 397 | 116.588 | 204.415 | 84.368 | 0.00 | 0.00 | AAAA | H |
| ATOM | 3776 | N | ILE | A | 398 | 116.938 | 206.557 | 86.657 | 1.00 | 50.00 | AAAA | N |
| ATOM | 3777 | CA | ILE | A | 398 | 116.787 | 207.714 | 87.533 | 1.00 | 50.00 | AAAA | C |
| ATOM | 3778 | C | ILE | A | 398 | 115.753 | 207.470 | 88.613 | 1.00 | 50.00 | AAAA | C |
| ATOM | 3779 | O | ILE | A | 398 | 115.990 | 207.756 | 89.775 | 1.00 | 50.00 | AAAA | O |
| ATOM | 3780 | CB | ILE | A | 398 | 116.501 | 208.986 | 86.718 | 1.00 | 50.00 | AAAA | C |
| ATOM | 3781 | CG1 | ILE | A | 398 | 117.762 | 209.345 | 85.931 | 1.00 | 50.00 | AAAA | C |
| ATOM | 3782 | CG2 | ILE | A | 398 | 116.109 | 210.179 | 87.602 | 1.00 | 50.00 | AAAA | C |
| ATOM | 3783 | CD1 | ILE | A | 398 | 117.536 | 210.510 | 84.965 | 1.00 | 50.00 | AAAA | C |
| ATOM | 3784 | H | ILE | A | 398 | 116.455 | 206.496 | 85.784 | 0.00 | 0.00 | AAAA | H |
| ATOM | 3785 | N | ILE | A | 399 | 114.612 | 206.892 | 88.186 | 1.00 | 50.00 | AAAA | N |
| ATOM | 3786 | CA | ILE | A | 399 | 113.576 | 206.617 | 89.184 | 1.00 | 50.00 | AAAA | C |
| ATOM | 3787 | C | ILE | A | 399 | 113.930 | 205.545 | 90.207 | 1.00 | 50.00 | AAAA | C |
| ATOM | 3788 | O | ILE | A | 399 | 113.399 | 205.529 | 91.307 | 1.00 | 50.00 | AAAA | O |
| ATOM | 3789 | CB | ILE | A | 399 | 112.212 | 206.326 | 88.539 | 1.00 | 50.00 | AAAA | C |
| ATOM | 3790 | CG1 | ILE | A | 399 | 112.224 | 205.021 | 87.737 | 1.00 | 50.00 | AAAA | C |
| ATOM | 3791 | CG2 | ILE | A | 399 | 111.792 | 207.524 | 87.680 | 1.00 | 50.00 | AAAA | C |
| ATOM | 3792 | CD1 | ILE | A | 399 | 110.868 | 204.622 | 87.153 | 1.00 | 50.00 | AAAA | C |
| ATOM | 3793 | H | ILE | A | 399 | 114.485 | 206.654 | 87.225 | 0.00 | 0.00 | AAAA | H |
| ATOM | 3794 | N | VAL | A | 400 | 114.874 | 204.668 | 89.804 | 1.00 | 50.00 | AAAA | N |
| ATOM | 3795 | CA | VAL | A | 400 | 115.350 | 203.670 | 90.763 | 1.00 | 50.00 | AAAA | C |
| ATOM | 3796 | C | VAL | A | 400 | 116.332 | 204.234 | 91.785 | 1.00 | 50.00 | AAAA | C |
| ATOM | 3797 | O | VAL | A | 400 | 116.258 | 203.942 | 92.971 | 1.00 | 50.00 | AAAA | O |
| ATOM | 3798 | CB | VAL | A | 400 | 115.877 | 202.399 | 90.053 | 1.00 | 50.00 | AAAA | C |
| ATOM | 3799 | CG1 | VAL | A | 400 | 117.180 | 202.596 | 89.275 | 1.00 | 50.00 | AAAA | C |
| ATOM | 3800 | CG2 | VAL | A | 400 | 116.004 | 201.231 | 91.031 | 1.00 | 50.00 | AAAA | C |
| ATOM | 3801 | H | VAL | A | 400 | 115.249 | 204.730 | 88.881 | 0.00 | 0.00 | AAAA | H |
| ATOM | 3802 | N | ILE | A | 401 | 117.217 | 205.116 | 91.274 | 1.00 | 50.00 | AAAA | N |
| ATOM | 3803 | CA | ILE | A | 401 | 118.106 | 205.875 | 92.158 | 1.00 | 50.00 | AAAA | C |
| ATOM | 3804 | C | ILE | A | 401 | 117.340 | 206.825 | 93.082 | 1.00 | 50.00 | AAAA | C |
| ATOM | 3805 | O | ILE | A | 401 | 117.725 | 207.097 | 94.212 | 1.00 | 50.00 | AAAA | O |
| ATOM | 3806 | CB | ILE | A | 401 | 119.170 | 206.592 | 91.297 | 1.00 | 50.00 | AAAA | C |
| ATOM | 3807 | CG1 | ILE | A | 401 | 120.133 | 205.548 | 90.718 | 1.00 | 50.00 | AAAA | C |
| ATOM | 3808 | CG2 | ILE | A | 401 | 119.979 | 207.639 | 92.077 | 1.00 | 50.00 | AAAA | C |
| ATOM | 3809 | CD1 | ILE | A | 401 | 121.164 | 206.144 | 89.754 | 1.00 | 50.00 | AAAA | C |
| ATOM | 3810 | H | ILE | A | 401 | 117.233 | 205.283 | 90.287 | 0.00 | 0.00 | AAAA | H |
| ATOM | 3811 | N | LEU | A | 402 | 116.197 | 207.283 | 92.541 | 1.00 | 50.00 | AAAA | N |
| ATOM | 3812 | CA | LEU | A | 402 | 115.327 | 208.188 | 93.285 | 1.00 | 50.00 | AAAA | C |
| ATOM | 3813 | C | LEU | A | 402 | 114.566 | 207.492 | 94.400 | 1.00 | 50.00 | AAAA | C |
| ATOM | 3814 | O | LEU | A | 402 | 114.419 | 208.011 | 95.499 | 1.00 | 50.00 | AAAA | O |
| ATOM | 3815 | CB | LEU | A | 402 | 114.403 | 208.895 | 92.285 | 1.00 | 50.00 | AAAA | C |
| ATOM | 3816 | CG | LEU | A | 402 | 113.537 | 210.049 | 92.800 | 1.00 | 50.00 | AAAA | C |
| ATOM | 3817 | CD1 | LEU | A | 402 | 113.301 | 211.078 | 91.695 | 1.00 | 50.00 | AAAA | C |
| ATOM | 3818 | CD2 | LEU | A | 402 | 112.205 | 209.583 | 93.394 | 1.00 | 50.00 | AAAA | C |
| ATOM | 3819 | H | LEU | A | 402 | 115.919 | 206.958 | 91.638 | 0.00 | 0.00 | AAAA | H |
| ATOM | 3820 | N | LEU | A | 403 | 114.107 | 206.271 | 94.061 | 1.00 | 50.00 | AAAA | N |
| ATOM | 3821 | CA | LEU | A | 403 | 113.387 | 205.465 | 95.047 | 1.00 | 50.00 | AAAA | C |
| ATOM | 3822 | C | LEU | A | 403 | 114.268 | 204.928 | 96.167 | 1.00 | 50.00 | AAAA | C |
| ATOM | 3823 | O | LEU | A | 403 | 113.813 | 204.603 | 97.255 | 1.00 | 50.00 | AAAA | O |
| ATOM | 3824 | CB | LEU | A | 403 | 112.643 | 204.330 | 94.335 | 1.00 | 50.00 | AAAA | C |
| ATOM | 3825 | CG | LEU | A | 403 | 111.656 | 203.559 | 95.220 | 1.00 | 50.00 | AAAA | C |
| ATOM | 3826 | CD1 | LEU | A | 403 | 110.548 | 204.461 | 95.771 | 1.00 | 50.00 | AAAA | C |
| ATOM | 3827 | CD2 | LEU | A | 403 | 111.091 | 202.336 | 94.498 | 1.00 | 50.00 | AAAA | C |
| ATOM | 3828 | H | LEU | A | 403 | 114.296 | 205.904 | 93.151 | 0.00 | 0.00 | AAAA | H |
| ATOM | 3829 | N | SER | A | 404 | 115.571 | 204.876 | 95.842 | 1.00 | 50.00 | AAAA | N |
| ATOM | 3830 | CA | SER | A | 404 | 116.552 | 204.492 | 96.852 | 1.00 | 50.00 | AAAA | C |
| ATOM | 3831 | C | SER | A | 404 | 116.890 | 205.606 | 97.840 | 1.00 | 50.00 | AAAA | C |
| ATOM | 3832 | O | SER | A | 404 | 117.516 | 205.390 | 98.871 | 1.00 | 50.00 | AAAA | O |
| ATOM | 3833 | CB | SER | A | 404 | 117.800 | 203.966 | 96.137 | 1.00 | 50.00 | AAAA | C |
| ATOM | 3834 | OG | SER | A | 404 | 118.671 | 203.301 | 97.057 | 1.00 | 50.00 | AAAA | O |
| ATOM | 3835 | H | SER | A | 404 | 115.878 | 205.165 | 94.936 | 0.00 | 0.00 | AAAA | H |
| ATOM | 3836 | HG | SER | A | 404 | 119.456 | 203.076 | 96.575 | 0.00 | 0.00 | AAAA | H |
| ATOM | 3837 | N | LEU | A | 405 | 116.444 | 206.821 | 97.470 | 1.00 | 50.00 | AAAA | N |
| ATOM | 3838 | CA | LEU | A | 405 | 116.689 | 207.966 | 98.343 | 1.00 | 50.00 | AAAA | C |
| ATOM | 3839 | C | LEU | A | 405 | 115.407 | 208.633 | 98.839 | 1.00 | 50.00 | AAAA | C |
| ATOM | 3840 | CB | LEU | A | 405 | 117.621 | 208.959 | 97.635 | 1.00 | 50.00 | AAAA | C |
| ATOM | 3841 | CG | LEU | A | 405 | 119.017 | 208.399 | 97.352 | 1.00 | 50.00 | AAAA | C |
| ATOM | 3842 | CD1 | LEU | A | 405 | 119.829 | 209.338 | 96.459 | 1.00 | 50.00 | AAAA | C |
| ATOM | 3843 | CD2 | LEU | A | 405 | 119.773 | 208.054 | 98.638 | 1.00 | 50.00 | AAAA | C |
| ATOM | 3844 | 1OCT | LEU | A | 405 | 114.381 | 207.959 | 98.916 | 1.00 | 50.00 | AAAA | O |
| ATOM | 3845 | 2OCT | LEU | A | 405 | 115.434 | 209.824 | 99.152 | 1.00 | 99.99 | AAAA | O |
| ATOM | 3846 | H | LEU | A | 405 | 115.907 | 206.945 | 96.636 | 0.00 | 0.00 | AAAA | H |
| END | | | | | | | | | | | | |

### REFERENCES

The entire contents of the following references are incorporated herein by reference:
Beeler, J. A., and van Wyke Coelingh, K. (1989). Neutralization epitopes of the F glycoprotein of respiratory syncytial virus: effect of mutation upon fusion function. J Virol 63(7), 2941-50.
Crowe, J. E., Firestone, C. Y., Crim, R., Beeler, J. A., Coelingh, K. L., Barbas, C. F., Burton, D. R., Chanock, R. M., and Murphy, B. R. (1998). Monoclonal antibody-resistant mutants selected with a respiratory syncytial virus-neutralizing human antibody fab fragment (Fab 19) define a unique epitope on the fusion (F) glycoprotein. Virology 252(2), 373-5.
Day, N. D., Branigan, P. J., Liu, C., Gutshall, L. L., Luo, J., Melero, J. A., Sarisky, R. T., and Del Vecchio, A. M. (2006). Contribution of cysteine residues in the extracellular domain of the F protein of human respiratory syncytial virus to its function. Virol J 3, 34.
Haas, J., Park, E. C., and Seed, B. (1996). Codon usage limitation in the expression of HIV-1 envelope glycoprotein. Curr Biol 6(3), 315-24.
Schwede, T., Kopp, J., Guex, N., and Peitsch, M. C. (2003). SWISS-MODEL: An automated protein homology-modeling server. Nucleic Acids Res 31(13), 3381-5.
Ternette, N., Stefanou, D., Kuate, S., Uberla, K., and Grunwald, T. (2007). Expression of RNA virus proteins by RNA polymerase II dependent expression plasmids is hindered at multiple steps. Virol J 4, 51.
Walsh, E. E., Falsey, A. R., and Sullender, W. M. (1998). Monoclonal antibody neutralization escape mutants of respiratory syncytial virus with unique alterations in the attachment (G) protein. J Gen Virol 79(Pt 3), 479-87.
Walsh, E. E., and Hruska, J. (1983). Monoclonal antibodies to respiratory syncytial virus proteins: identification of the fusion protein. J Virol 47(1), 171-7.
Yin, H. S., Paterson, R. G., Wen, X., Lamb, R. A., and Jardetzky, T. S. (2005). Structure of the uncleaved ectodomain of the paramyxovirus (hPIV3) fusion protein. Proc Natl Acad Sci USA 102(26), 9288-93.

Yin, H. S., Wen, X., Paterson, R. G., Lamb, R. A., and Jardetzky, T. S. (2006). Structure of the parainfluenza virus 5 F protein in its metastable, prefusion conformation. Nature 439(7072), 38-44.

1. An isolated soluble fusion (F) protein of a virus in the paramyxovirus family, wherein the soluble fusion protein lacks a transmembrane domain and a cytoplasmic tail domain and comprises a CRAC1 domain, and wherein the soluble fusion protein is in a pre-triggered conformation and can be triggered when exposed to a triggering event.

2. The soluble fusion protein of claim 1, wherein the virus is a pneumovirus.

3. The soluble fusion protein of claim 1, wherein the virus is human respiratory syncytial virus (RSV).

4. The soluble fusion protein of claim 3, comprising a sequence that is at least 85% identical to amino acids 27-109 and 137-522 of SEQ ID NO. 1.

5. The soluble fusion protein of claim 3, comprising a sequence that is at least 90% identical to amino acids 27-109 and 137-522 of SEQ ID NO. 1.

6. The soluble fusion protein of claim 3, comprising a sequence that is at least 95% identical to amino acids 27-109 and 137-522 of SEQ ID NO. 1.

7. The soluble fusion protein of claim 3, comprising a sequence that is 100% identical to amino acids 27-109 and 137-522 of SEQ ID NO. 1.

8. The soluble fusion protein of claim 4, wherein the CRAC domain has the sequence VLDLKNYIDK, SEQ ID NO: 20.

9. The soluble fusion protein of claim 4, wherein the CRAC domain has the sequence VLDLKNYIDR, SEQ ID NO: 42.

10. The soluble fusion protein of claim 4, wherein the CRAC domain has the sequence VLDIKNYIDK, SEQ ID NO: 43.

11. The soluble fusion protein of claim 4, wherein the CRAC domain has the sequence ILDLKNYIDK, SEQ ID NO: 44.

12. The soluble fusion protein of claim 4, wherein the CRAC domain has the sequence VLDLKNYINNR, SEQ ID NO: 45.

13. The soluble fusion protein of claim 4, wherein the CRAC domain has the sequence VRELKDFVSK, SEQ ID NO: 46.

14. The soluble fusion protein of claim 4, wherein the CRAC domain has the sequence LKTLQDFVNDEIR, SEQ ID NO: 47.

15. The soluble fusion protein of claim 4, wherein the CRAC domain has the sequence VQDYVNK, SEQ ID NO: 48.

16. The soluble fusion protein of claim 4, wherein the CRAC domain has the sequence VNDQFNK, SEQ ID NO: 49.

17. The soluble fusion protein of claim 1, comprising a pep27 domain.

18. The soluble fusion protein of claim 1, wherein the protein lacks a GCNt clamp.

19. The soluble fusion protein of claim 1, wherein the protein comprises a C-terminal clamp comprising two cysteine residues.

20. The soluble fusion protein of claim 1, comprising a detection tag.

21. The soluble fusion protein of claim 1, wherein the pre-triggered conformation substantially conforms to the atomic coordinates represented in Table 4.

22. A functional fragment of an RSV soluble fusion protein, comprising a first and a second peptide linked to form a dimer peptide, wherein the first and second peptide comprise, respectively, a sequence that is at least 90% identical to amino acids 37-69 and 156-440 of SEQ ID NO: 1, and wherein the second peptide includes a CRAC1 domain.

23. A method of screening for a candidate paramyxovirus antiviral agent, comprising the steps of:
(i) contacting a test agent with an isolated soluble F protein of a paramyxovirus according to claim 1,
(ii) detecting a structural indicator of the soluble pre-triggered F protein, wherein a change in the structural indicator of the soluble pre-triggered F protein in the presence of the test agent as compared to the absence of the test agent indicates that the agent is a candidate antiviral agent against the paramyxovirus.

24. A method of screening for a candidate paramyxovirus antiviral agent, comprising the steps of:
(i) contacting a test agent with a soluble F protein of the paramyxovirus according to claim 1 to form a test sF protein;
(ii) exposing the test sF protein to a triggering event; and
(iii) assessing a structural indicator of the test sF protein before and after exposure to the triggering event, wherein an absence of a change in the structural indicator of the test sF protein after exposure to the triggering event indicates that the agent is a candidate antiviral agent against the paramyxovirus.

25. The method of claim 23, wherein the paramyxovirus is a pneumovirus.

26. The method of claim 23, wherein the paramyxovirus is human respiratory syncytial virus (RSV).

27. The method of claim 23, wherein the soluble F protein comprises a sequence that is at least 85% identical to amino acids 27-109 and 137-522 of SEQ ID NO. 1.

28. The method of claim 23, wherein the soluble F protein comprises a sequence that is at least 90% identical to amino acids 27-109 and 137-522 of SEQ ID NO. 1.

29. The method of claim 23, wherein the soluble F protein comprises a sequence that is at least 95% identical to amino acids 27-109 and 137-522 of SEQ ID NO. 1.

30. The method of claim 23, wherein the soluble F protein comprises a sequence that is 100% identical to amino acids 27-109 and 137-522 of SEQ ID NO. 1.

31. The method of claim 23, wherein the soluble F protein comprises a CRAC domain that has the sequence VLDLKNYIDK, SEQ ID NO: 20.

32. The method of claim 23, wherein the soluble F protein comprises a CRAC domain that has the sequence VLDLKNYIDR, SEQ ID NO: 42.

33. The method of claim 23, wherein the soluble F protein comprises a CRAC domain that has the sequence VLDIKNYIDK, SEQ ID NO: 43.

34. The method of claim 23, wherein the soluble F protein comprises a CRAC domain that has the sequence ILDLKNYIDK, SEQ ID NO: 44.

35. The method of claim 23, wherein the soluble F protein comprises a CRAC domain that has the sequence VLDLKNYINNR, SEQ ID NO: 45.

36. The method of claim 23, wherein the soluble F protein comprises a CRAC domain that has the sequence VRELKDFVSK, SEQ ID NO: 46.

37. The method of claim 23, wherein the soluble F protein comprises a CRAC domain that has the sequence LKTLQDFVNDEIR, SEQ ID NO: 47.

38. The method of claim 23, wherein the soluble F protein comprises a CRAC domain that has the sequence VQDYVNK, SEQ ID NO: 48.

39. The method of claim 23, wherein the soluble F protein comprises a CRAC domain that has the sequence VNDQFNK, SEQ ID NO: 49.

40. The method of claim 23, wherein the soluble F comprises a pep27 domain.

41. The method of claim 23, wherein the soluble F protein lacks a GCNt clamp.

42. The method of claim 23, wherein the soluble F protein comprises a C-terminal clamp comprising two cysteine residues.

43. The method of claim 23, wherein the steps are performed in the absence of an attachment protein.

44. The method of claim 23, wherein the structural indicator comprises one or more of the following: (i) circular dichroism (CD) spectrum; (ii) fluorescence emission; (iii) resonance Raman spectrum; (iv) fluorescence indicative of hydrophobic dye binding; (v) liposome association; (vi) hydrophobic association; (vii) split GFP; (vii) FRET; and (viii) antibody binding.

45. The method of claim 24, wherein the triggering event is exposure to heat or to a lipid membrane.

46. A method of screening for a candidate antiviral agent against human RSV, comprising the steps of:
(i) contacting a test agent with a functional fragment of a soluble pre-triggered F protein of RSV, wherein the functional fragment comprises a first and a second peptide linked to form a dimer peptide, wherein the first and second peptides comprise, respectively, a sequence that is at least 90% identical to amino acids 37-69 and 156-440 of SEQ ID NO: 1, and wherein the second peptide includes a CRAC1 domain;
(ii) detecting a structural indicator of the functional fragment, wherein a change in the structural indicator of the functional fragment in the presence of the test agent as compared to the absence of the test agent indicates that the agent is a candidate antiviral agent against RSV.

47. A method of screening for a candidate antiviral agent against human RSV, comprising the steps of:
(i) contacting a test agent with a functional fragment of a soluble pre-triggered F protein of RSV to form a test sF protein, wherein the functional fragment comprises a first and a second peptide linked to form a dimer peptide, wherein the first and second peptides comprise, respectively, a sequence that is at least 90% identical to amino acids 37-69 and 156-440 of SEQ ID NO: 1;
(ii) exposing the test sF protein to a triggering event; and
(iii) assessing a structural indicator of the test sF protein before and after exposure to the triggering event, wherein an absence of a change in the structural indicator of the test sF protein after exposure to the triggering event indicates that the agent is a candidate antiviral agent against RSV.

48. The method of claim 46, wherein the first and second peptides comprise a sequence that is at least 95% identical to amino acids 37-69 and 156-440 of SEQ ID NO: 1, respectively.

49. The method of claim 46, wherein the first and second peptides comprise a sequence that is at least 100% identical to amino acids 37-69 and 156-440 of SEQ ID NO: 1, respectively.

50. The method of claim 46, wherein the functional fragment comprises a sequence that is at least 90% identical to amino acids 27-36 of SEQ ID NO: 1.

51. The method of claim 46, wherein the functional fragment comprises a sequence that is at least 90% identical to amino acids 70-109 of SEQ ID NO: 1.

52. The method of claim 46, wherein the functional fragment comprises a sequence that is at least 90% identical to amino acids 110-136 of SEQ ID NO: 1.

53. The method of claim 46, wherein the functional fragment comprises a sequence that is at least 90% identical to amino acids 137-155 of SEQ ID NO: 1.

54. The method of claim 46, wherein the functional fragment comprises a sequence that is at least 90% identical to amino acids 441-522 of SEQ ID NO: 1.

55. The method of claim 46, wherein the functional fragment comprises a CRAC domain that has the sequence VLDLKNYIDK, SEQ ID NO: 20.

56. The method of claim 46, wherein the functional fragment comprises a CRAC domain that has the sequence VLDLKNYIDR, SEQ ID NO: 42.

57. The method of claim 46, wherein the functional fragment comprises a CRAC domain that has the sequence VLDIKNYIDK, SEQ ID NO: 43.

58. The method of claim 46, wherein the functional fragment comprises a CRAC domain that has the sequence ILDLKNYIDK, SEQ ID NO: 44.

59. The method of claim 46, wherein the functional fragment lacks a C-terminal GCNt clamp.

60. The method of claim 46, wherein the functional fragment comprises a C-terminal clamp comprising two cysteine residues.

61. The method of claim 46, wherein the structural indicator comprises one or more of the following: (i) circular dichroism (CD) spectrum; (ii) fluorescence emission; (iii) resonance Raman spectrum; (iv) fluorescence indicative of hydrophobic dye binding; (v) liposome association; (vi) hydrophobic association; (vii) split GFP; (vii) FRET; and (viii) antibody binding.

62. The method of claim 47, wherein the triggering event comprises exposure to heat or to a lipid membrane.
